(19) United States
(12) Patent Application Publication
(10) Pub. No.: US 2026/0146251 A1
(43) Pub. Date: May 28, 2026

(54) DOUBLE STRANDED RNAi AGENTS, COMPOSITIONS AND METHODS OF USE

(71) Applicant: Novartis AG, Basel (CH)

(72) Inventors: Stefan HAEMMIG, Bottmingen (CH); Caitlin Jeanette HAGEN, Wilmington, MA (US); Juerg HUNZIKER, Aarau (CH); Ge TAN, Bottmingen (CH); John Wesley TRAUGER, Boston, MA (US); Jan WEILER, Loerrach (DE)

(21) Appl. No.: 19/398,805

(22) Filed: Nov. 24, 2025

Related U.S. Application Data
(60) Provisional application No. 63/724,671, filed on Nov. 25, 2024.

(51) Int. Cl. C12N 15/113

(52) U.S. Cl. CPC C12N 15/1137; C12Y 304/21061; C12N 2310/14; C12N 2310/315; C12N 2310/321; C12N 2310/322; C12N 2310/351

(57) ABSTRACT

Disclosed are, inter alia, double stranded RNAi (dsRNAi) agents inhibiting expression of proprotein convertase subtilisin kexin 9 (PCSK9), for example, human PCSK9, compositions including the same, and methods of treatment using the same.

### CROSS-REFERENCE TO RELATED APPLICATIONS

The present application claims priority and benefit to the U.S. Patent Application No. 63/724,671 filed Nov. 25, 2024, the disclosure of which is incorporated herein by reference in its entirety.

### SEQUENCE LISTING

The instant application contains a Sequence Listing which has been submitted electronically in XML format and is hereby incorporated by reference in its entirety. Said XML copy, created on Oct. 10, 2025, is named PAT059857-US-SECO1_SL.xml and is 4,029,476 bytes in size.

### TECHNICAL FIELD

The present disclosure provides, inter alia, double stranded RNAi (dsRNAi) agents inhibiting expression of proprotein convertase subtilisin kexin 9 (PCSK9), for example, human PCSK9, compositions including the same, and methods of treatment using the same.

### BACKGROUND

Cumulative low-density lipoprotein cholesterol (LDL-C) exposure in the arterial wall is a major cause of atherosclerotic cardiovascular disease (ASCVD). The level of LDL-C in the arterial wall can be controlled (e.g., lowered) by modulating cholesterol homeostasis (e.g., cholesterol biosynthesis in liver cells) and upregulating LDL-C uptake from the blood.

Proprotein convertase subtilisin kexin 9 (PCSK9) is a member of the subtilisin serine protease family and is involved in cholesterol metabolism and homeostasis. A liver cell uptakes LDL-C via low-density lipoprotein receptor (LDLR) to directly remove such atherosclerotic lipoproteins from the plasma via uptake into a liver cell. If the level of LDLR decreases, LDL-C level increases in circulation, and the elevated circulating LDL-C level causes increased deposition in the arteries of LDL particles and the cholesterol they carry, which promotes the formation and progression of atherosclerotic plaques in the artery. PCSK9 binds to hepatic LDLR to induce endocytosis and lysosomal degradation of LDLR in the liver cell, thereby reducing LDL-C uptake by LDLR. PCSK9 inhibitors have been clinically proven to reduce LDLR degradation by inhibiting PCSK9 expression/function and lower LDL-C levels in the general population.

However, there is still an unmet need in the art for LDL-C lowering treatments that have increased potency and/or durability of action.

### SUMMARY OF THE INVENTION

Provided herein are, inter alia, compounds that can inhibit expression of PCSK9 in a subject, for example, e.g., in liver cells of a subject.

In an aspect, the disclosure provides a double stranded RNAi (dsRNAi) agent comprising:
(i) a sense strand comprising a nucleotide sequence selected from SEQ ID Nos. 3 to 381;
(ii) an antisense strand forming a duplex with the sense strand and comprising a nucleotide sequence selected from SEQ ID Nos. 382 to 760.

In an aspect, the disclosure provides a double stranded RNAi (dsRNAi) agent comprising:
(i) a sense strand comprising a nucleotide sequence selected from SEQ ID Nos. 761 to 776;
(ii) an antisense strand forming a duplex with the sense strand and comprising a nucleotide sequence selected from SEQ ID Nos. 777 to 792.

In some embodiments, one or more nucleotides in the sense strand and the antisense strand are modified nucleotides. In some embodiments, all nucleotides in the sense strand and the antisense strand are modified nucleotides.

In some embodiments, each of the modified nucleotides independently comprises one or more modifications selected from a 2′-deoxy modification, a 2′-O-alkyl modification, a 2′-halo modification, a threofuranosyl nucleotide (TNA) modification, a 2′-5′-linkage modification, a conformationally restricting modification, an abasic modification, a 2′-amino-modification, a 2′-O-allyl modification, 2′-C-alkyl modification, a 2′-O-alkoxyalkyl modification, a morpholino modification, a phosphoramidate modification, a non-natural nucleobase modification, a modification in a tetrahydropyran, a modification containing a 1,5-anhydrohexitol, a modification containing a cyclohexenyl, a modification containing a phosphorothioate group, a modification containing a 5′-vinyl-phosphonate, a modification containing a 5′-phosphate, a modification to form a thermally destabilizing nucleotide, a glycol nucleic acid (GNA) modification, and a 2-O—(N-methylacetamide) modification.

In some embodiments, each of the modified nucleotides independently comprises one or more modifications selected from 2′-deoxy modification, 2′-O-alkoxyalkyl modification, 2′-O-alkyl modification, 2′-O-allyl modification, 2′-C-allyl modification, 2′-halo modification, modification containing a non-natural nucleobase, GNA modification, and TNA modification.

In some embodiments, the dsRNAi agent comprises a 3′-phosphorothioate (PS) modification.

In some embodiments, each of the modified nucleotides independently comprises one or more modifications selected from 2′-deoxy modification, 2′-O-methyl (2′-OMe) modification, 2′-fluoro (2′-F) modification, 2′-O-methoxyethyl (2′-MOE) modification, the modification containing a non-natural nucleobase, TNA, GNA, 3′-phosphorothioate (PS) modification, and 5′-vinyl-phosphonate (5′-VP) modification.

In some embodiments, the sense strand comprises one or two 2′-MOE modifications positioned at the 1st and/or 2nd nucleotides from the 5′ end.

In some embodiments, the sense strand comprises one or two 2′-MOE modifications positioned at the 1st and/or 2nd nucleotides from the 3′ end.

In some embodiments, the sense strand comprises one or two TNAs positioned at the 1st and/or 2nd nucleotides from the 5′ end.

In some embodiments, the sense strand comprises one or two TNAs positioned at the 1st and/or 2nd nucleotides from the 3′ end.

In some embodiments, the antisense strand comprises a 5′-VP modification at the 1st nucleotide from the 5′ end.

In some embodiments, the antisense strand comprises a 5′-VP-2′-OMe modification at the 1st position from the 5′ end.

In some embodiments, each of the sense strand and the antisense strand independently comprises two, three, four, five or six 2′-F modified nucleotides.

In some embodiments, the sense strand comprises one or two 3′-PS modifications at the 1st and/or 2nd nucleotides from the 5′ end.

In some embodiments, the sense strand comprises one or two 3′-PS modifications at the 1st and/or 2nd nucleotides from the 3′ end.

In some embodiments, the antisense strand comprises one or two 3′-PS modifications at the 1st and/or 2nd nucleotides from the 5′ end, and/or one or two 3′-PS modifications at the 1st and/or 2nd nucleotides from the 3′ end.

In certain aspects, the sense strand is 21 nucleotides in length and the antisense strand is 23 nucleotides in length.

In some embodiments, the sense strand comprises one to four 2′-MOE modifications positioned at the 1st, 2nd, 20th, and/or 21st nucleotides from the 5′ end.

In some embodiments, the sense strand does not comprise a 2′-MOE modification at the 3rd to 19th positions from the 5′ end.

In some embodiments, the sense strand comprises one to four TNAs positioned at the 1st, 2nd, 20th, and/or 21st nucleotides from the 5′ end.

In some embodiments, the sense strand does not comprise a 2′-MOE modifications and TNA at the 3rd to 19th positions from the 5′ end.

In some embodiments, the sense strand comprises two, three, or four 2′-F modifications positioned at the 7th, 9th, 10th, and/or 11th nucleotides from the 5′ end.

In some embodiments, the sense strand comprises one or two 2′-deoxy modifications positioned at the 10th and/or 11th nucleotides from the 5′ end.

In some embodiments, the sense strand comprises (i) 2′-F modifications positioned at the 7th, 9th, and 10th nucleotides from the 5′ end and (ii) a 2′-deoxy modification positioned at the 11th nucleotide from the 5′ end.

In some embodiments, the remaining nucleotides in the sense strand comprise 2′-OMe modifications.

In some embodiments, the antisense strand comprises a 5′-(E)-VP modification at the 1st nucleotide from the 5′ end.

In some embodiments, the antisense strand comprises a 5′-(E)-VP-2′-OMe modification at the 1st nucleotide from the 5′ end.

In some embodiments, the antisense strand comprises two, three, or four 2′-F modifications positioned at the 2nd, 6th, 14th, and/or 16th nucleotides from the 5′ end.

In some embodiments, the antisense strand comprises 2′-F modifications positioned at the 2nd, 6th, 14th, and 16th nucleotides from the 5′ end.

In some embodiments, the antisense strand comprises 2′-F modifications positioned at the 2nd, 6th, 14th, and 16th nucleotides from the 5′ end and a TNA positioned at the 3rd nucleotide from the 5′ end.

In some embodiments, the antisense strand comprises 2′-F modifications positioned at the 2nd, 6th, 14th, and 16th nucleotides from the 5′ end and a TNA, GNA or 2′-deoxy modification positioned at the 5th nucleotide from the 5′ end.

In some embodiments, the antisense strand comprises 2′-F modifications positioned at the 2nd, 14th, and 16th nucleotides from the 5′ end and a TNA, GNA or 2′-deoxy modification positioned at the 6th nucleotide from the 5′ end.

In some embodiments, the antisense strand comprises 2′-F modifications positioned at the 2nd, 6th, 14th, and 16th nucleotides and a TNA, GNA or 2′-deoxy modification positioned at the 7th nucleotide.

In some embodiments, the remaining nucleotides in antisense strand comprise 2′-OMe modified modifications.

In some embodiments, the sense strand comprises one to eight 3′-PS group at the 1st, 2nd, 3rd, 4th, 17th, 18th, 19th and/or 20th nucleotides from the 5′ end.

In some embodiments, the antisense strand comprises one to eight 3′-PS group at the 1st, 2nd, 3rd, 4th, 19th, 20th, 21st and/or 22nd nucleotides from the 5′ end.

In some embodiments, at least one of the 3′-PS groups in each sense strand and antisense strand has a stereopure Rp configuration.

In some embodiments, at least one of the 3′-PS groups in each sense strand and antisense strand has a stereopure Sp configuration.

In certain aspects, the double stranded RNAi (dsRNAi) agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

In some embodiments, the dsRNAi agent comprises a ligand.

In some embodiments, the ligand comprises a N-acetylgalactosamine (GalNAc) moiety.

In some embodiments, the ligand has a structure of:

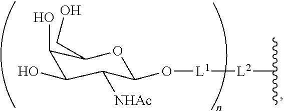

wherein:
each L¹ is independently a linker which may be same or different in each occurrence;
L² is a linker;
n is an integer from 1 to 3; and
[symbol] is an attachment point to the sense strand or the antisense strand, or to a conjugate linker conjugated to the sense strand or the antisense strand.

In some embodiments, the ligand comprises the following structure of

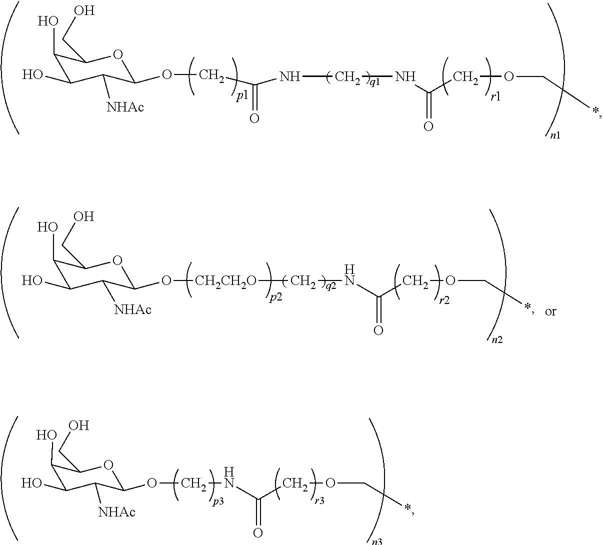

wherein:
each p1, p2, p3, q1, q2, r1, r2 and r3 is independently an integer from 0 to 12;
each n1, n2, and n3 is independently an integer from 1 to 3; and
“*” is an attachment point to L².

In some embodiments, the ligand has a structure of:

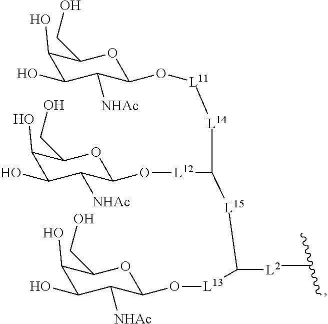

wherein:
each L¹¹, L¹², L¹³, L¹⁴, and L¹⁵ is an independently a linker;
L² is a linker;
[symbol] is an attachment point to the sense strand or the antisense strand, or to a conjugate linker conjugated to the sense strand or the antisense strand.

In some embodiments, the ligand has a structure of:

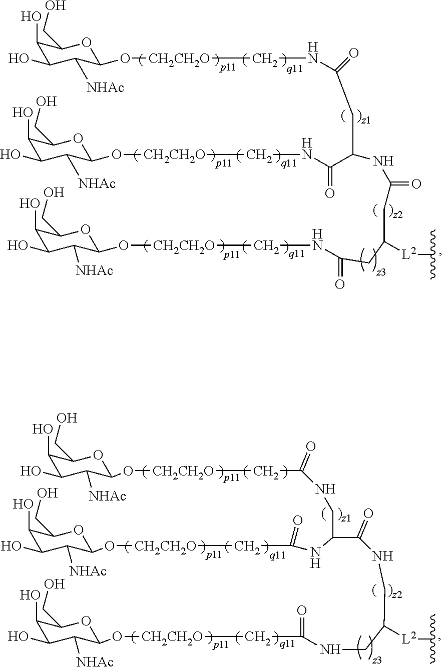

wherein:
each p11 and q11 is independently an integer from 0 to 12;
each z1, z2, and z3 is independently an integer of 0 to 12; and
[symbol] is an attachment point to the sense strand or the antisense strand, or to a conjugate linker conjugated to the sense strand or the antisense strand.

In some embodiments, the ligand comprises the following structure:

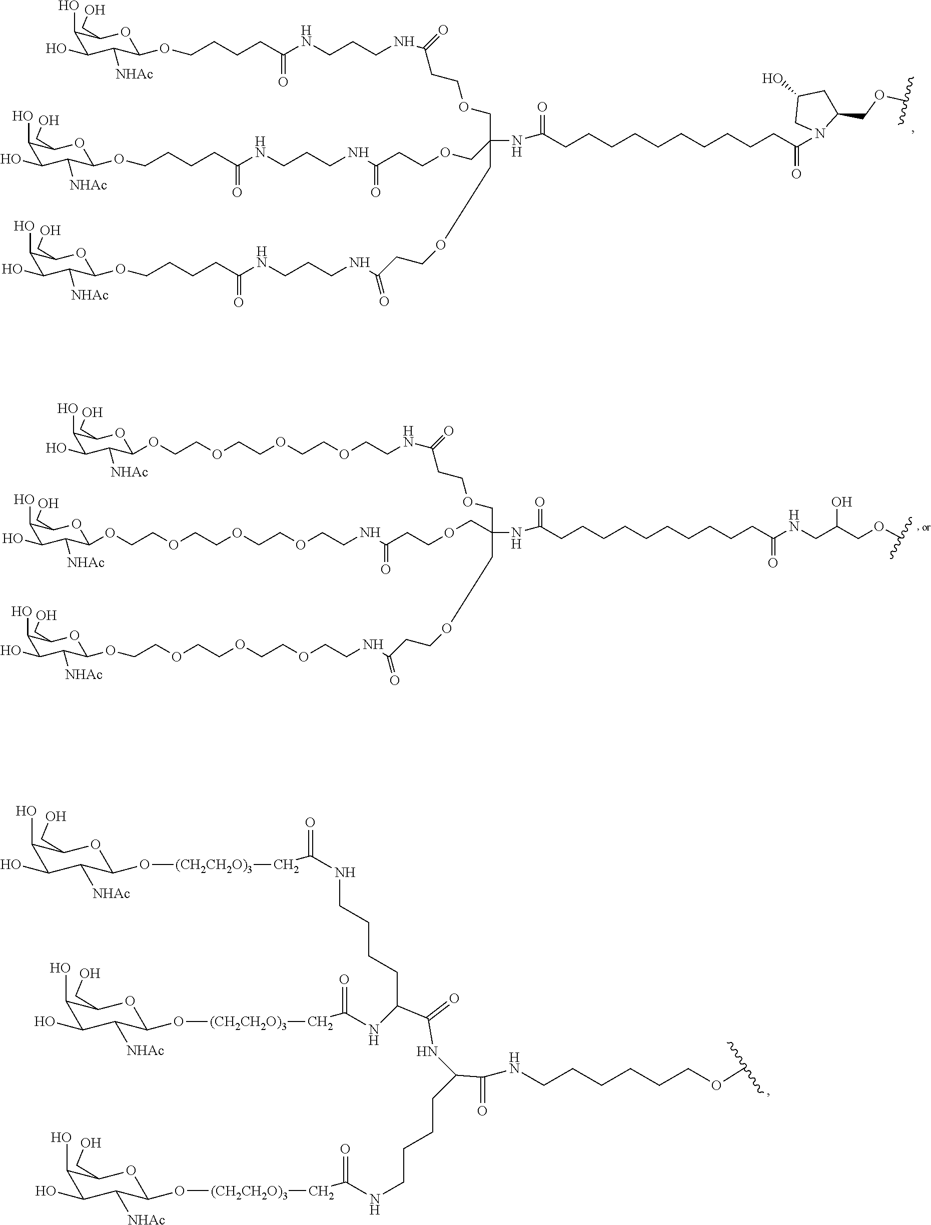

wherein
[symbol] is an attachment point to the sense strand or the antisense strand or to a conjugate linker conjugated to the sense strand or the antisense strand.

In some embodiments, the ligand is conjugated to 3′ end of the sense strand to form the following structure:

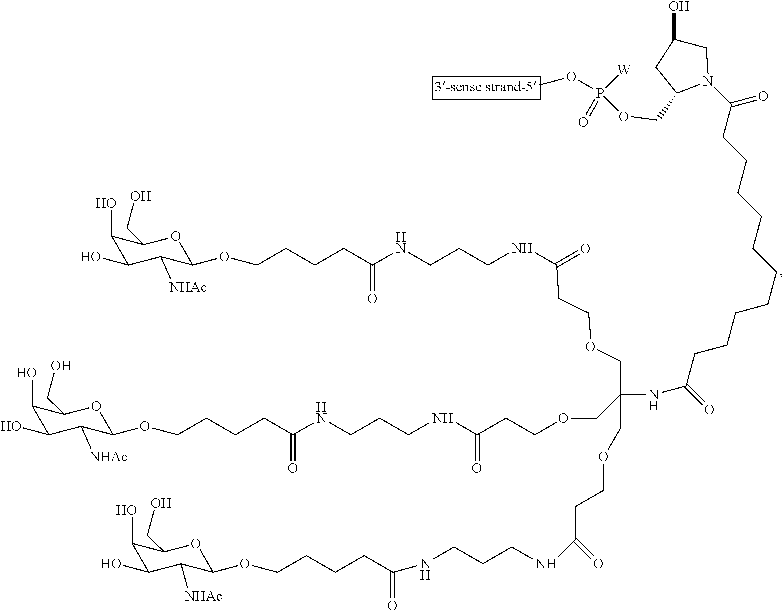

wherein W is —OH or —SH.

In some embodiments, the ligand is conjugated to 5′ end of the sense strand to form the following structure:

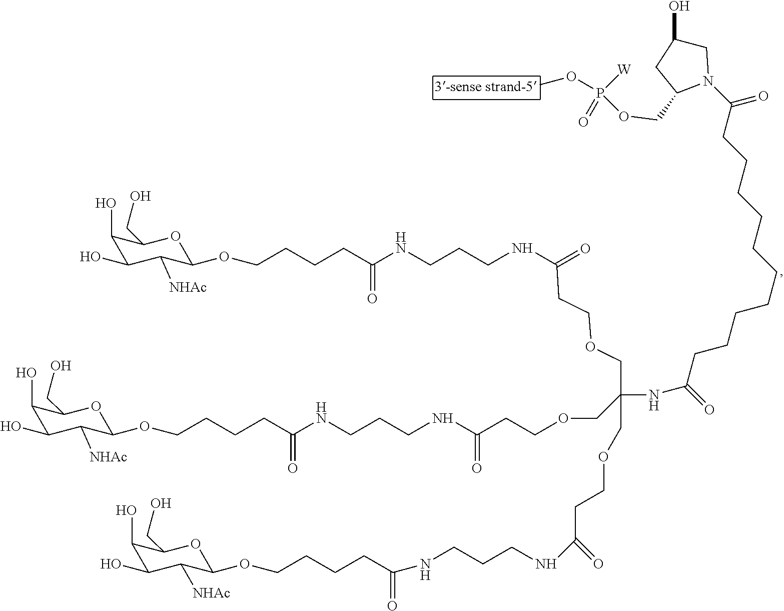

or a pharmaceutically acceptable salt thereof, wherein W is —OH or —SH.

In some embodiments, W is —OH.

In an aspect, a double stranded RNAi (dsRNAi) agent comprises:
(a) a sense strand consisting of SEQ ID NO: 906, and
   an antisense strand consisting of SEQ ID NO: 963;
(b) a sense strand consisting of SEQ ID NO: 907, and
   an antisense strand consisting of SEQ ID NO: 964;
(c) a sense strand consisting of SEQ ID NO: 947, and
   an antisense strand consisting of SEQ ID NO: 1004;
(d) a sense strand consisting of SEQ ID NO: 948, and
   an antisense strand consisting of SEQ ID NO: 1005;
(e) a sense strand consisting of SEQ ID NO: 949, and
   an antisense strand consisting of SEQ ID NO: 1006;
or
(f) a sense strand consisting of SEQ ID NO: 950, and
   an antisense strand consisting of SEQ ID NO: 1007;
wherein the ligand (L96) is conjugated to the 3′ end of the sense strand to form the following schematic:

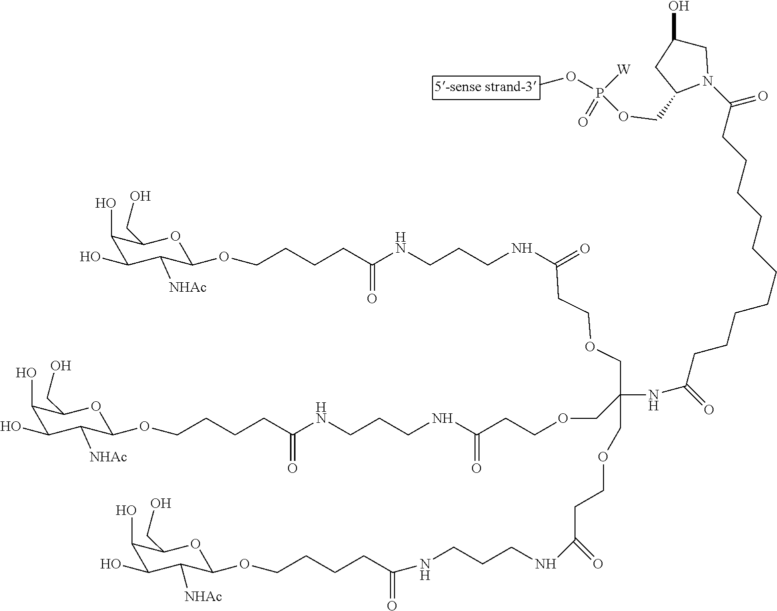

or a pharmaceutically acceptable salt thereof, wherein W is —OH.

In some embodiments, the dsRNAi agent is in a pharmaceutically acceptable salt form.

In some embodiments, the pharmaceutically acceptable salt is a sodium salt.

In an aspect, the disclosure provides a pharmaceutical composition comprising the dsRNAi agent as described herein, or a pharmaceutically acceptable salt thereof, and a pharmaceutically acceptable carrier.

In some embodiments, the composition is in an aqueous solution form.

In an aspect, the disclosure provides a method of inhibiting PCSK9 expression in a cell, the method comprising:
(a) contacting the cell with the dsRNAi agent as described herein or a pharmaceutically acceptable salt thereof, or the pharmaceutical composition as described herein; and
(b) maintaining the cell produced in step (a) for a time sufficient to obtain degradation of the mRNA transcript of a PCSK9 gene, thereby inhibiting expression of the PCSK9 gene in the cell.

In an aspect, the disclosure provides a method of lowering a level of low-density lipoprotein cholesterol (LDL-C) in a subject in need thereof, comprising administering to the subject the dsRNAi agent as described herein or a pharmaceutically acceptable salt thereof, or the pharmaceutical composition as described herein.

In an aspect, the disclosure provides a method of treating lipidemia mediated by PCSK9 expression in a subject in need thereof, comprising administering to the subject the dsRNAi agent as described herein or a pharmaceutically acceptable salt thereof, or the pharmaceutical composition as described herein.

In an aspect, the disclosure provides a method of treating or preventing atherosclerotic cardiovascular disease (ASCVD) in a subject in need thereof, comprising administering to the subject the dsRNAi agent as described herein, or a pharmaceutically acceptable salt thereof, or the pharmaceutical composition as described herein.

In an aspect, the disclosure provides a method of reducing or preventing cardiovascular event in a subject in need thereof, comprising administering to the subject the dsRNAi agent as described herein or a pharmaceutically acceptable salt thereof, or the pharmaceutical composition as described herein.

In some embodiments, the cardiovascular event is cardiovascular death, non-fatal myocardial infarction (MI), non-fatal ischemic stroke, urgent coronary revascularization, coronary heart disease (CHD) death, or any combination thereof.

In an aspect, the disclosure provides a method of reducing or preventing a major limb adverse event (MALE) in a subject in need thereof, comprising administering to the subject the dsRNAi agent as described herein or a pharmaceutically acceptable salt thereof, or the pharmaceutical composition as described herein.

In some embodiments, the MALE is acute lower limb ischemia, lower limb amputation due to ischemia, urgent lower limb revascularization for ischemia, or any combination thereof.

In an aspect, the disclosure provides a method of inhibiting PCSK9 expression in a cell, the method comprising:
(i) contacting the cell with a dsRNAi agent, wherein the dsRNAi agent comprises:
   (a) a sense strand comprising SEQ ID NO: 800, and
      an antisense strand comprising SEQ ID NO: 853;
   (b) a sense strand comprising SEQ ID NO: 801, and
      an antisense strand comprising SEQ ID NO: 854;
   (c) a sense strand comprising SEQ ID NO: 806, and
      an antisense strand comprising SEQ ID NO: 859;
   (d) a sense strand comprising SEQ ID NO: 811, and
      an antisense strand comprising SEQ ID NO: 864;
   (e) a sense strand comprising SEQ ID NO: 813, and
      an antisense strand comprising SEQ ID NO: 866;
   or
   (f) a sense strand comprising SEQ ID NO: 830, and
      an antisense strand comprising SEQ ID NO: 883,
(ii) maintaining the cell produced in step (a) for a time sufficient to obtain degradation of the mRNA transcript of a PCSK9 gene, thereby inhibiting expression of the PCSK9 gene in the cell.

In an aspect, the disclosure provides a method of lowering a level of low-density lipoprotein cholesterol (LDL-C) in a subject in need thereof, comprising administering to the subject a dsRNAi agent, wherein the dsRNAi agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

In an aspect, the disclosure provides a method of treating lipidemia mediated by PCSK9 expression in a subject in need thereof, comprising administering to the subject a dsRNAi agent, wherein the dsRNAi agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

In an aspect, the disclosure provides a method of treating or preventing atherosclerotic cardiovascular disease (ASCVD) in a subject in need thereof, comprising administering to the subject a dsRNAi agent, wherein the dsRNAi agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

In an aspect, the disclosure provides a method of reducing or preventing cardiovascular event in a subject in need thereof, comprising administering to the subject a dsRNAi agent, wherein the dsRNAi agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

In some embodiments, the cardiovascular event is cardiovascular death, non-fatal myocardial infarction (MI), non-fatal ischemic stroke, urgent coronary revascularization, coronary heart disease (CHD) death, or any combination thereof.

In an aspect, the disclosure provides a method of reducing or preventing a major limb adverse event (MALE) in a subject in need thereof, comprising administering to the subject a dsRNAi agent, wherein the dsRNAi agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

In some embodiments, the MALE is acute lower limb ischemia, lower limb amputation due to ischemia, urgent lower limb revascularization for ischemia, or any combination thereof.

In some embodiments, the dsRNAi agent further comprises a ligand comprising the following structure:

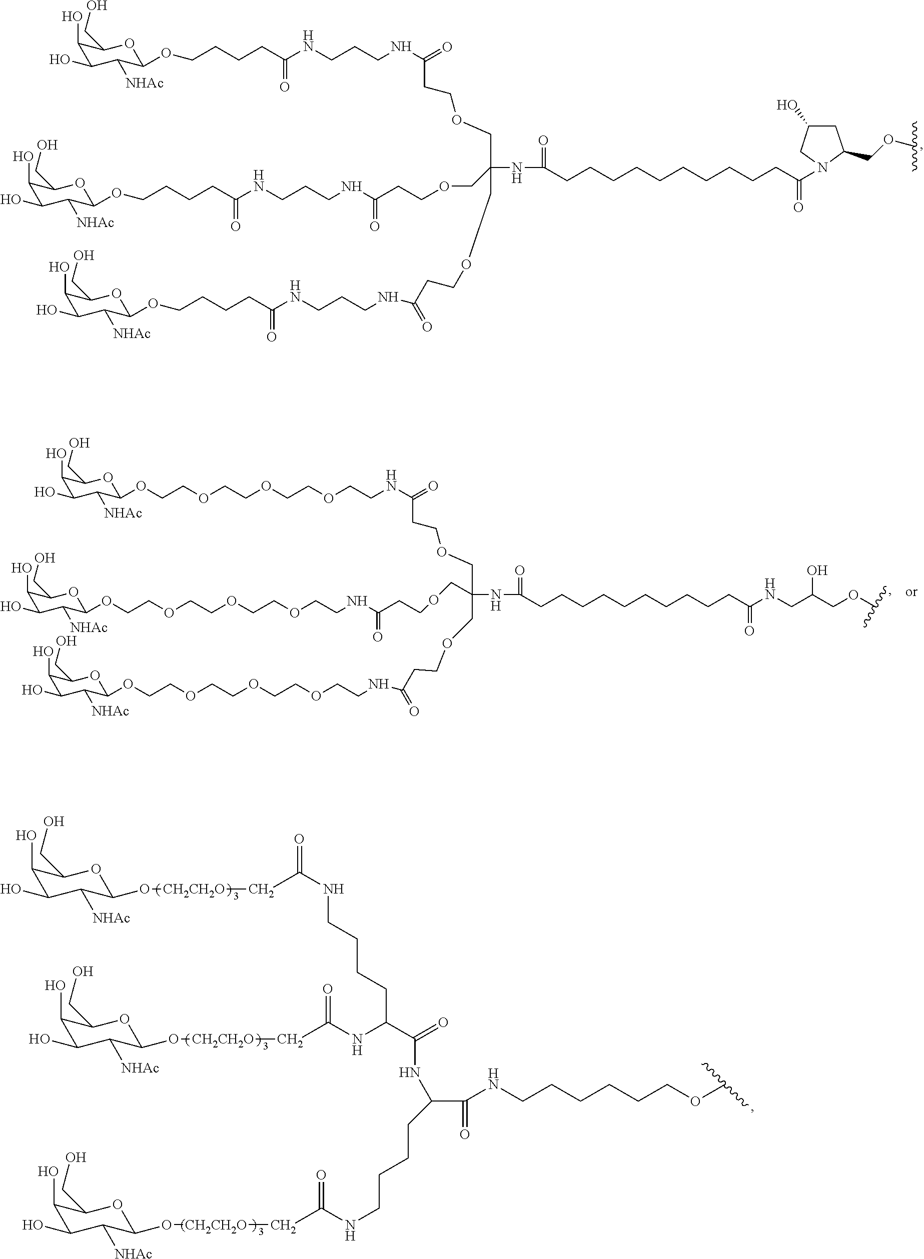

wherein
[symbol] is an attachment point to the sense strand or the antisense strand or to a conjugate linker conjugated to the sense strand or the antisense strand.

In some embodiments, the subject is a human.

In some embodiments, the subject has or is diagnosed with hyperlipidemia, hypercholesterolemia, hypertriglyceridemia, mixed hyperlipidemia, congestive heart disease (CHD) or atherosclerosis.

In some embodiments, the dsRNAi agent or the pharmaceutical composition is administered to the subject subcutaneously or intravenously.

In an aspect, the disclosure provides a kit comprising the dsRNAi agent as described herein, or a pharmaceutically acceptable salt thereof, or the pharmaceutical composition as described herein.

In some embodiments, the kit further comprises an applicator.

In some embodiments, the applicator is a syringe.

In some embodiments, the applicator is a pre-filled syringe.

Other aspects of the invention are disclosed infra.

### BRIEF DESCRIPTION OF DRAWINGS

FIGS. 1A-1B. Effects of a single 3 mg/kg subcutaneous administration of PCSK9 siRNAs D1, D2, D3, D4, D5, or D6 to humanized PCSK9 mice on (FIG. 1A) plasma PCSK9 levels over time and (FIG. 1A) liver human PCSK9 mRNA level, liver siRNA level, and liver RISC loading (i.e., the level of siRNA incorporated into RISC) on day 56 post-dose.

FIGS. 2A-2B. Effects of a single 3 mg/kg subcutaneous administration of PCSK9 siRNAs D7, D8, D9, or D10 to humanized PCSK9 mice on (FIG. 3A) plasma PCSK9 levels over time and (FIG. 3B) liver human PCSK9 mRNA level, liver siRNA level, and liver RISC loading (i.e., the level of siRNA incorporated into RISC) on day 77post-dose.

FIGS. 3A-3B. Effects of a single 3 mg/kg subcutaneous administration of PCSK9 siRNAs D1, D4, D11, or D12 to humanized PCSK9 mice on (FIG. 3A) plasma PCSK9 levels over time and (FIG. 3B) liver human PCSK9 mRNA level, liver siRNA level, and liver RISC loading (i.e., the level of siRNA incorporated into RISC) on day 63 post-dose.

FIGS. 4A-4B. Effects of a single 3 mg/kg subcutaneous administration of PCSK9 siRNAs D4, D14, D15, D16, or D17 to humanized PCSK9 mice on (FIG. 4A) plasma PCSK9 levels over time and (FIG. 4B) liver human PCSK9 mRNA level, liver siRNA level, and liver RISC loading (i.e., the level of siRNA incorporated into RISC) on day 77 post-dose.

FIGS. 5A-5B. Effects of a single 3 mg/kg subcutaneous administration of PCSK9 siRNAs D4, D18, D19, D20, D21, D22, or D23 to humanized PCSK9 mice on (FIG. 5A) plasma PCSK9 levels over time and (FIG. 5B) liver human PCSK9 mRNA level, liver siRNA level, and liver RISC loading (i.e., the level of siRNA incorporated into RISC) on day 77 post-dose.

FIGS. 6A-6B. Effects of a single 3 mg/kg subcutaneous administration of PCSK9 siRNAs D4, D13, D24, D25, D26, D27, or D28 to humanized PCSK9 mice on (FIG. 6A) plasma PCSK9 levels over time and (FIG. 6B) liver human PCSK9 mRNA level, liver siRNA level, and liver RISC loading (i.e., the level of siRNA incorporated into RISC) on day 63 post-dose.

FIGS. 7A-7B. Effects of a single 3 mg/kg subcutaneous administration of PCSK9 siRNAs D25, D29, D30, D31, D32, D33, or D34 to humanized PCSK9 mice on (FIG. 7A) plasma PCSK9 levels over time and (FIG. 7B) liver human PCSK9 mRNA level, liver siRNA level, and liver RISC loading (i.e., the level of siRNA incorporated into RISC) on day 64 post-dose.

FIGS. 8A-8B. Effects of a single 3 mg/kg subcutaneous administration of PCSK9 siRNAs D4, D15, D35, D36, D37, or D38 to humanized PCSK9 mice on (FIG. 8A) plasma PCSK9 levels over time and (FIG. 8B) liver human PCSK9 mRNA level, liver siRNA level, and liver RISC loading (i.e., the level of siRNA incorporated into RISC) on day 77 post-dose.

FIG. 9 shows example PCSK9 siRNA compounds (dsRNAi agents) as described herein. Figure discloses SEQ ID NOS 963, 906, 964, 907, 1004, 947, 1005, 948, 1006, 949, 1007, and 950, respectively, in order of appearance (from top to bottom).

FIGS. 10A-10C. Effects of a single 3 mg/kg subcutaneous administration of PCSK9 siRNAs D15, D38, D39, D40, D41, D42, D43, D44, D45, D46, D47, or D48 to humanized PCSK9 mice on plasma PCSK9 levels over time (FIGS. 10A-10B) and liver siRNA level and liver RISC loading (i.e., the level of siRNA incorporated into RISC) on day 77 post-dose (FIG. 10C).

FIGS. 11A-11B. Effects of a single 3 mg/kg subcutaneous administration of PCSK9 siRNAs D4, D19, D49, or D50 to humanized PCSK9 mice on plasma PCSK9 levels over time (FIG. 11A) and liver human PCSK9 mRNA level, liver siRNA level, and liver RISC loading (i.e., the level of siRNA incorporated into RISC) on day 77 post-dose (FIG. 11B).

FIGS. 12A-12C. Effects of a single subcutaneous administration of PCSK9 siRNAs D7 (3 or 6 mg/kg), D8 (3 or 6 mg/kg), D51 (3 mg/kg), D52 (3 mg/kg), D53 (3 mg/kg), or D54 (1, 3, or 6 mg/kg) to humanized PCSK9 mice on plasma PCSK9 levels over time (FIGS. 12A-12B) and liver human PCSK9 mRNA level, liver siRNA level, and liver RISC loading (i.e., the level of siRNA incorporated into RISC) on day 77 post-dose (FIG. 12C).

FIGS. 13A-13C. Effects of a single 3 mg/kg subcutaneous administration of PCSK9 siRNAs D38, D55, D42, D43, D44, D56, D57, D58, or D59 to humanized PCSK9 mice on plasma PCSK9 levels over time (FIGS. 13A-13B) and liver human PCSK9 mRNA level, liver siRNA level, and liver RISC loading (i.e., the level of siRNA incorporated into RISC) on day 77 post-dose (FIG. 13C).

FIGS. 14A-14B. Effects of a single 0.3, 1, or 3 mg/kg subcutaneous administration of PCSK9 siRNAs D7 or D52 to humanized PCSK9 mice on plasma PCSK9 levels over time (FIG. 14A) and liver human PCSK9 mRNA level, liver siRNA level, and liver RISC loading (i.e., the level of siRNA incorporated into RISC) on day 77 post-dose (FIG. 14B).

FIGS. 15A-15B. Effects of a single 3 mg/kg subcutaneous administration of PCSK9 siRNAs D52, D60, D61, or D62 to humanized PCSK9 mice on plasma PCSK9 levels over time (FIG. 15A) and liver siRNA level and liver RISC loading (i.e., the level of siRNA incorporated into RISC) on day 77 post-dose (FIG. 15B).

FIG. 16. Effects of a single 3 mg/kg subcutaneous administration of PCSK9 siRNAs D8 or D63 to humanized PCSK9 mice on plasma PCSK9 levels over time.

FIG. 17. Effects of PCSK9 siRNAs D7, D8, or D52 transfection at various concentrations into cultured Hep3B cells on PCSK9 mRNA levels.

FIG. 18. Results of in vitro specificity profiling of the PCSK9 siRNAs D52, D60, D61, or D62 in Hep3B cells.

FIGS. 19A-19B. Effects of a single 3 mg/kg subcutaneous administration of PCSK9 siRNAs D8, D51, D52, D53, or D54 to obese cynomolgus monkeys on plasma PCSK9 levels over time (FIG. 19A) and serum low density lipoprotein cholesterol (LDL-C) levels over time (FIG. 19B).

### DETAILED DESCRIPTION

### Definitions

Unless defined otherwise, all technical terms, scientific terms, abbreviations, chemical structures, and chemical formulae used herein have the same meaning as is commonly understood by one of ordinary skill in the art. The chemical structures and formulae set forth herein are constructed according to the standard rules of chemical valency known in the chemical arts. All patents, applications, published applications, and other publications referenced herein are incorporated by reference in their entirety unless stated otherwise.

All patents, applications, published applications, and other publications referenced herein are incorporated by reference in their entirety unless stated otherwise. Unless otherwise indicated, conventional methods of mass spectroscopy, NMR, HPLC, protein chemistry, biochemistry, recombinant DNA techniques, and pharmacology are employed.

Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components. As used herein, the term “a,” “an,” “the” and similar terms used in the context of the present invention (especially in the context of the claims) are to be construed to cover both the singular and plural unless otherwise indicated herein or clearly contradicted by the context.

Unless otherwise indicated, all numbers, values, and/or expressions referring to nucleotide lengths, inhibition, activities, dosages, contents, and formulations used herein are to be understood as modified in all instances by the term “about” as such numbers are inherently approximations that are reflective of, among other things, the various uncertainties of measurement encountered in obtaining such values. Further, unless specifically stated or obvious from context, as used herein, the term “about” is understood as within a range of normal tolerance in the art, for example within 2 standard deviations of the “mean. “About” may be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The term “nucleic acid” means a compound containing at least two nucleotide monomers covalently linked together. Nucleic acids include polynucleotides and oligonucleotides, including double-stranded oligonucleotides and single-stranded oligonucleotides, and modified versions thereof.

The term “nucleotide” means a compound including a nucleoside and a phosphate group (or phosphodiester linkage) that are covalently attached at 5′ position or 3′ position of the pentofuranosyl sugar (e.g., ribose or deoxyribose). In certain aspects, the nucleotide is a ribonucleotide (RNA) having the ribose as the pentofuranosyl sugar. In certain aspects, a nucleotide is a deoxyribonucleotide (DNA) having the deoxyribose (2′-deoxyribose) as the pentofuranosyl sugar. Unless otherwise specifically indicated, when referring a “nucleotide” in a chain of nucleotides (e.g., oligonucleotides), e.g., X₁ to X₂₁ and X_{1′} to X_{23′}, a nucleotide is meant by a nucleoside and a phosphate group (or phosphodiester linkage) that is covalently attached at 3′ position of the pentofuranosyl sugar (e.g., ribose or deoxyribose).

The term “nucleoside” means a monomer consisting of a nucleobase and a pentofuranosyl sugar (e.g., ribose or deoxyribose). A nucleoside including a ribose sugar ring has to a structure of

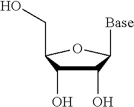

or a pharmaceutically acceptable salt thereof, and a nucleotide including a deoxyribose sugar ring has a structure of

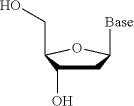

or a pharmaceutically acceptable salt, wherein in each structure, “Base” is a nucleobase.

The term “nucleobase” or “base,” as used herein, means the heterocyclic base moiety of a nucleoside or nucleotide. Non-limiting examples of nucleobases includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), 7-methylguanine or a derivative thereof (e.g., 7-methylguanine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In some embodiments, the nucleobase is adenine, guanine, hypoxanthine, xanthine, theobromine, caffeine, uric acid, or isoguanine, which may be optionally substituted or modified. In some embodiments, the nucleobase is

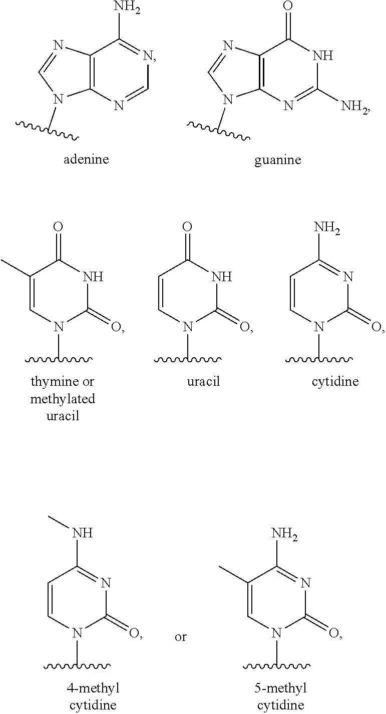

which may be optionally substituted or modified, wherein “ [symbol] ” denotes the point of attachment to a pentofuranosyl sugar ring (e.g., 1′ position).

The term “phosphate,” or “phosphate group” as used herein a chemical species made of one phosphorus atom and four oxygen atoms

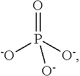

or esters, salts, or acids thereof. In certain aspects, when the phosphate groups are positioned between adjacent nucleosides in RNA or DNA strand and form a “backbone” of the oligonucleotides, these terms “phosphate,” or “phosphate group” may be interchangeable used as “phosphate group,” “phosphate linkage,” “phosphodiester linkage,” or “linkage.” For example, the phosphate or phosphodiester linkage in the backbone of RNA or DNA may have the structures of

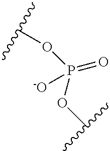

or esters, salts (e.g., pharmaceutically acceptable salts), or acids

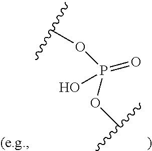 [SMILES: *OP(=O)(O)O*]

thereof, wherein “ [symbol] ” denotes the point of attachment to pentofuranosyl sugar rings (e.g., 5′ and 3′ positions) in adjacent nucleosides. In certain aspects, a variant of a phosphate or phosphodiester linkage, e.g., phosphorothioate (PS) linkage, can replace a phosphate group (or phosphodiester linkage) in the backbone and connect two adjacent nucleosides. In certain aspects, a variant of a phosphate or phosphodiester linkage, e.g., phosphorothioate (PS) linkage or vinyl phosphonate (VP) group, may be additionally attached at 3′ end or 5′ end of the oligonucleotides (e.g., RNA or DNA), e.g., 3′-OH or 5′-OH position of the terminal pentofuranosyl sugar (e.g., ribose or deoxyribose), so as to act as chemically or biologically functional group. In certain aspects, a variant of phosphate or phosphodiester linkage may also be referred as a phosphorus-derived internucleoside linkage that includes at least one phosphorus atom in the backbone.

Unless otherwise indicated herein, an unmodified RNA (or “ribonucleotide”) in a chain of nucleotides (e.g., mRNA, rRNA, or sense strand or antisense strand of siRNA) as disclosed refers to a structure of

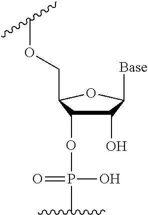

or a pharmaceutically acceptable salt thereof. Likewise, an unmodified DNA (or “deoxyribonucleotides”) in a chain of nucleotides (e.g., genomic DNA or cDNA) as disclosed herein specifically refers to a structure of

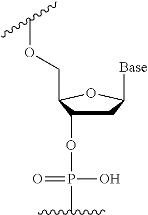

or a pharmaceutically acceptable salt thereof. In each structure “Base” is a nucleobase and [symbol] is an attachment point to the adjacent nucleotides.

Unless otherwise indicated herein, when an unmodified RNA is the first nucleotide from the 5′ end of an RNA chain (e.g., mRNA, or sense strand or antisense strand of siRNA), that nucleotide has a structure of

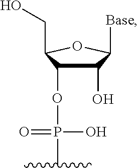

or a pharmaceutically acceptable salt thereof. Likewise, when an unmodified DNA is the first nucleotide from the 5′ end of a DNA chain (e.g., genomic DNA or cDNA), that nucleotide has a structure of

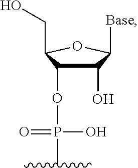

or a pharmaceutically acceptable salt thereof. In each structure “Base” is a nucleobase and [symbol] is an attachment point (5′ oxygen) to the adjacent nucleotides. Alternatively but equivalently, for example, the first nucleotide from the 5′ end of an RNA chain (e.g., mRNA, or sense strand or antisense strand of siRNA), that nucleotide has a structure of

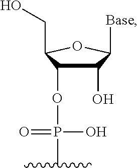

or a pharmaceutically acceptable salt thereof and the first nucleotide from the 5′ end of a DNA chain (e.g., genomic DNA or cDNA), that nucleotide has a structure of

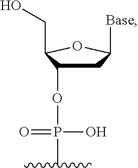

or a pharmaceutically acceptable salt thereof, when [symbol] is an attachment point (5′ oxygen) to the adjacent nucleotides.

Unless otherwise indicated herein, when an unmodified RNA is the first nucleotide from the 3′ end of an RNA chain (e.g., mRNA, or sense strand or antisense strand of siRNA), that nucleotide has a structure of

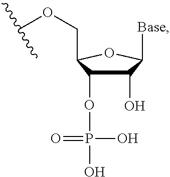

or a pharmaceutically acceptable salt. Likewise, when an unmodified DNA is the first nucleotide from the 3′ end of a DNA chain (e.g., genomic DNA or cDNA), that nucleotide has a structure of

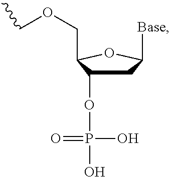

or a pharmaceutically acceptable salt thereof. In certain embodiments, when an unmodified RNA is the first nucleotide from the 3′ end of an RNA chain (e.g., mRNA, or sense strand or antisense strand of siRNA) that nucleotide does not include 3′ end phosphate group or phosphodiester linkage, for example, which has been removed during hydrolysis or synthesis, has a structure of

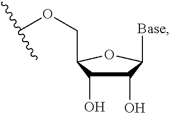

or a pharmaceutically acceptable salt. Likewise, when an unmodified DNA is the first nucleotide from the 3′ end of a DNA chain (e.g., genomic DNA or cDNA), that nucleotide does not include 3′ end phosphate group, for example, which has been removed during hydrolysis or synthesis, has a structure of

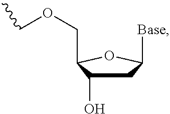

or a pharmaceutically acceptable salt thereof. In each structure “Base” is a nucleobase and [symbol] is an attachment point (e.g., phosphorus of the phosphate linkage) to the adjacent nucleotides.

A code “A”, “G”, “C”, or “U” presented in a sequence list as disclosed herein stand for a RNA nucleotide that contains adenine, guanine, cytosine, or uracil as a base, respectively. A code “dA”, “dG”, “dC” or “dT” presented in a sequence list as disclosed herein stand for a DNA nucleotide that contains adenine, guanine, cytosine, and thymine as a base, respectively. In some embodiments, the code “T” may be present in a RNA sequence then it may refer to a nucleotide (e.g. modified nucleotide) that thymine as a base.

The term “oligonucleotide” means a shorter length nucleic acid, e.g. of less than 100 nucleotides in length. Oligonucleotides may be single-stranded or double-stranded. In some embodiments, an oligonucleotide may include naturally occurring ribonucleotides, naturally occurring deoxyribonucleotides, and/or nucleotides having one or more modifications to a naturally occurring terminus, sugar, nucleobase, and/or internucleoside linkage. Non-limiting examples of oligonucleotides include double-stranded oligonucleotides (e.g., dsRNA), single-stranded oligonucleotides (e.g., single stranded RNA or ssRNA), antisense oligonucleotides (“ASO”), small interfering RNA (siRNA), microRNA mimics, short hairpin RNAs (shRNA), single-strand small interfering RNA (ssRNAi), RNaseH oligonucleotides, anti-microRNA oligonucleotides, steric blocking oligonucleotides, exon-skipping oligonucleotides, CRISPR guide RNAs, and aptamers. In certain aspects, the oligonucleotide is a dsRNA and each strand has a length less than 100 nucleotides (“nt”), less than 90 nt, less than 80 nt, less than 70 nt, less than 60 nt, less than 50 nt, less than 40 nt, less than 35 nt, less than 30 nt, less than 28 nt, less than 26 nt, less than 25 nt, less than 24 nt, less than 23 nt, less than 22 nt, less than 21 nt, less than 20 nt, less than 19 nt, less than 18 nt, less than 17 nt, less than 16 nt, or 15 nt.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript (mRNA) via an RNA-induced silencing complex (RISC) pathway. An RNAi agent directs the sequence-specific degradation of mRNA through a process and thereafter inhibits expression of the gene encoded by the mRNA in a cell in vivo, e.g., in a subject (e.g., any vertebrate, mammal, or human).

The term “small interfering RNA” or “siRNA” means a double-stranded oligonucleotide (dsRNA) formed with two anti-parallel, and partially, substantially or fully complementary nucleic acid strands (e.g., a first strand and a second strand; or a “sense” strand and an “antisense” strand), which interferes with the expression of genes in a sequence-specific manner by facilitating mRNA degradation before translation through the RNA interference pathway. In some embodiments, depending on the context, the first strand can be a “guide” or antisense strand, and the second strand can be a “passenger” or sense strand. In some embodiments, depending on the context, the “first” strand can be a passenger or sense strand, and the “second” strand can be a guide or antisense. In certain aspects, an “RNAi agent” or “siRNA agent,” as used herein, refers a double-stranded RNA (dsRNA) with or without a ligand or other conjugate, and may be interchangeably used with a term “double stranded RNAi agent (dsRNAi agent),” or “dsRNA agent.” In certain aspects of the disclosure, the term “siRNA” can be used to describe a dsRNA with specific nucleotide sequences (unmodified or modified nucleotide sequences), without a ligand or other conjugate.

The term “antisense strand,” as used herein, refers an oligonucleotide (e.g., RNA) of an siRNA or a dsRNAi that is complementary (e.g., partially, substantially, or fully complementary) to the target mRNA and is incorporated into the RNA-induced silencing complex (RISC) to direct gene silencing in a sequence-specific manner through the RNA interference pathway. An antisense strand may also be referred to as the “guide strand.” In some embodiments, the antisense strand may have a length from 15-30 nt, 15-26 nt, 15-23 nt, 15-22 nt, 15-21 nt, 15-20 nt, 15-19 nt, 15-18 nt, 15-17 nt, 18-30 nt, 18-26 nt, 18-23 nt, 18-22 nt, 18-21 nt, 18-20 nt, 19-30 nt, 19-26 nt, 19-23 nt, 19-22 nt, 19-21 nt, 19-20 nt, 19 nt, 20-30 nt, 20-26 nt, 20-25 nt, 20-24 nt, 20-23 nt, 20-22 nt, 20-21 nt, 20 nt, 21-30 nt, 21-26 nt, 21-25 nt, 21-24 nt, 21-23 nt, 21-22 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, or 36 nt.

The term “sense strand,” as used herein, refers an oligonucleotide that is complementary (e.g., partially, substantially, or fully complementary) to the antisense strand. The sense strand is typically degraded following incorporation of the antisense strand into RISC. The sense strand may also be referred to as the “passenger strand.” In some embodiments, the sense strand may have a length from 15-30 nt, 15-26 nt, 15-23 nt, 15-22 nt, 15-21 nt, 15-20 nt, 15-19 nt, 15-18 nt, 15-17 nt, 18-30 nt, 18-26 nt, 18-23 nt, 18-22 nt, 18-21 nt, 18-20 nt, 19-30 nt, 19-26 nt, 19-23 nt, 19-22 nt, 19-21 nt, 19-20 nt, 19 nt, 20-30 nt, 20-26 nt, 20-25 nt, 20-24 nt, 20-23 nt, 20-22 nt, 20-21 nt, 20 nt, 21-30 nt, 21-26 nt, 21-25 nt, 21-24 nt, 21-23 nt, 21-22 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, or 36 nt.

The term “complementary” means that a nucleotide (e.g., RNA or DNA) or a sequence of nucleotides are capable of base pairing non-covalently via hydrogen bonding with another nucleotide or sequence of nucleotides. As described herein and commonly known in the art the complementary (matching) nucleotide of adenosine is thymidine or uridine and the complementary (matching) nucleotide of guanosine is cytidine. The complementarity of sequences may be partial, in which only some of the nucleic acids match according to base pairing, or complete, where all the nucleic acids match according to base pairing. For example, two sequences that are complementary to each other, may have a specified percentage of nucleotides that participate in nucleobase-pairing (i.e., about 50% complementarity, preferably 50%, 55%, 65%, 70%, 75%, 80%, 85%, 90%, 91%, 92%, 93%, 94%, 95%, 96%, 97%, 98%, 99%, or greater complementarity over a specified region). In some embodiments, two sequences are partially complementary when the percentage of nucleotides that participate in nucleobase-pairing is about 50%, about 55%, about 65%, about 70%, about 75%, or about 80%, or ranges from about 50% to about 80%. In some embodiments, two sequences are substantially complementary when the percentage of nucleotides that participate in nucleobase-pairing is about 80%, about 81%, about 82%, about 83%, about 84%, about 85%, about 86%, about 87%, about 88%, about 89%, about 90%, about 91%, about 92%, about 92%, about 93%, about 94%, or about 95%, or ranges from about 80% to about 95%.

Examples of complementary (e.g., partially, substantially, or fully complementary) sequences are sense and antisense sequences, wherein the sense sequence contains complementary (e.g., partially, substantially, or fully complementary) nucleotides to the antisense sequence and thus forms the complement of the antisense sequence. In certain aspects, a sense strand and an antisense strand of a double-stranded oligonucleotide (e.g., double stranded RNA) are substantially or fully complementary over their entire lengths. In some embodiments, a sense strand and an antisense strand of dsRNA are substantially or fully complementary over the entire length of the double-stranded region of the siRNA, and one or both termini of either strand comprises single-stranded nucleotides.

Another examples of complementary (e.g., partially, substantially, or fully complementary) sequences are an antisense strand and its target mRNA sequence. In certain aspects, an antisense strand is substantially or fully complementary to its target mRNA. For example, the complementary (e.g., partially, substantially, or fully complementary) sequences may be between an antisense strand and a coding region of the target mRNA, or a non-coding sequence of the target mRNA. In certain aspects, an antisense strand is substantially, or fully complementary to its target mRNA to reduce or eliminate off-target profile for and to improve down-regulation of the target gene (e.g., gene of the target mRNA sequence).

The terms “identical” or percent “identity,” in the context of two or more nucleic acids or polypeptide sequences, refer to two or more sequences or subsequences that are the same or have a specified percentage of amino acid residues or nucleotides that are the same (i.e., at least 60% identity, or at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or within a range defined by any of two of the preceding values, identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). This definition also refers to, or may be applied to, the complement of a test sequence. The definition also includes sequences that have deletions and/or additions, as well as those that have substitutions. As described below, the preferred algorithms can account for gaps, insertions and the like. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

As used herein, “target sequence” or “target gene” refer to a contiguous portion of the nucleotide sequence of an mRNA molecule formed during the transcription of a gene including mRNA that is a product of RNA processing of a primary transcription product. The target portion of the sequence will be at least long enough to serve as a substrate for RNAi-directed cleavage at or near that portion. For example, the target sequence will generally be from 9-36 nucleotides (“nt”) in length, e.g., 15-30 nt in length, including all sub-ranges therebetween. As non-limiting examples, the target sequence may have a length from 15-30 nt, 15-26 nt, 15-23 nt, 15-22 nt, 15-21 nt, 15-20 nt, 15-19 nt, 15-18 nt, 15-17 nt, 18-30 nt, 18-26 nt, 18-23 nt, 18-22 nt, 18-21 nt, 18-20 nt, 19-30 nt, 19-26 nt, 19-23 nt, 19-22 nt, 19-21 nt, 19-20 nt, 19 nt, 20-30 nt, 20-26 nt, 20-25 nt, 20-24 nt, 20-23 nt, 20-22 nt, 20-21 nt, 20 nt, 21-30 nt, 21-26 nt, 21-25 nt, 21-24 nt, 21-23 nt, 21-22 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, or 36 nt.

The term “ligand,” as used herein, refers to a compound or moiety that can impose characteristics to provide additional properties, e.g., affinity or cell delivery efficiency, to an RNAi (e.g., dsRNAi) as described herein. The ligand may be coupled or conjugated directly to the RNAi (e.g., sense strand or antisense strand of dsRNA), or indirectly to the RNAi agent (e.g., sense strand or antisense strand of dsRNA) via an intervening linker (“linker”). When a ligand is conjugated or coupled indirectly to the RNAi (e.g., dsRNA) via a linker, the ligand may be formed of a core moiety (e.g., targeting moiety) that has specific function to provide affinity or efficacy and the linker that provides merely an optimal distance, e.g., between the core moiety and the RNAi agent (dsRNA). In certain aspects, the term “ligand” embraces the ligand in combination with the linker. Examples of ligands or targeting moieties thereof may include, but not be limited to, one or more selected from a synthetic or natural compound, a peptide, an antibody, a carbohydrate (e.g., sugar moiety), or an additional nucleic acid.

The term “modified nucleotide” means a nucleotide having one or more modifications relative to a naturally occurring nucleotide, e.g., RNA. The modified nucleotide may be selected over an unmodified form because of desirable properties such as, for example, enhanced cellular uptake, enhanced affinity for other oligonucleotides or nucleic acid targets, increased stability in the presence of nucleases, and/or reduced immune stimulation. In certain aspects, the modification may be present in at least one of (i) an internucleoside linkage (“linkage”), (ii) a nucleobase, and (iii) a sugar moiety of the nucleotide. In certain aspects, the modification is present in the internucleoside linkage, e.g., by chemically modifying a phosphate (or phosphodiester) linkage or replacing a phosphate (or phosphodiester) linkage with other linking groups. In certain aspects, the modification is present in a sugar moiety, i.e., ribose ring, by substituting hydroxyl group on 2′ position of the ribose ring with other chemical group or by replacing a ring structure with other heterocycloalkyl or cycloalkyl, glycol group having a structure of

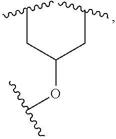 [SMILES: *CC(C*)O*]

bicyclic or bridged ring on the ribose such as locked nucleic acid (LNA) having a structure of

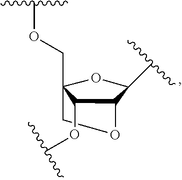 [SMILES: *OC[C@@]12COC([C@H](*)O1)[C@H]2O*]

or the like. In certain aspects, the modification is present in a nucleobase (e.g., A, G, C, T, or U) by chemical modification in a nucleobase by replacing the nucleobase with other moiety, for example, by replacing one naturally occurring nucleobase with another naturally occurring nucleobase. In certain aspects, a modified nucleotide may contain a modification in a sugar moiety and an unmodified phosphate (or phosphodiester) linkage. In certain aspects, a modified nucleotide may have a modification in a sugar moiety but with an unmodified nucleobase. In certain aspects, a modified nucleotide may have a modification in a sugar moiety and a nucleobase. In certain aspects, a modified nucleotide may have a modification in a sugar moiety and a phosphate (or phosphodiester) linkage. In certain aspects, a modified nucleotide may have a modification in a sugar moiety, a phosphate (or phosphodiester) linkage and a nucleobase. In certain aspects, a modified nucleotide may have an unmodified sugar moiety and an unmodified phosphate (or phosphodiester) linkage. In certain aspects, a modified nucleotide may have an unmodified sugar moiety and an unmodified nucleobase. In certain aspects, a modified nucleotide may have an unmodified sugar moiety and a modified nucleobase. In certain aspects, a modified nucleotide may have an unmodified sugar moiety and a modified phosphate (or phosphodiester) linkage. In certain aspects, a modified nucleotide may have a modified sugar moiety, a modified phosphate (or phosphodiester) linkage and a modified nucleobase.

The term “modified phosphate group,” or “modified phosphodiester linkage” as used herein refers to a chemical group in place of a phosphate group (or phosphodiester linkage) in a nucleotide as being attached to the 3′ end (3′ carbon) of the pentofuranosyl group.

The terms “identical” or percent “identity,” in the context of two or more nucleic acids or polypeptide sequences, refer to two or more sequences or subsequences that are the same or have a specified percentage of amino acid residues or nucleotides that are the same (i.e., at least 60% identity, or at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or within a range defined by any of two of the preceding values, identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). This definition also refers to, or may be applied to, the complement of a test sequence. The definition also includes sequences that have deletions and/or additions, as well as those that have substitutions. As described below, the preferred algorithms can account for gaps, insertions and the like. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

Throughout the disclosure, nucleotide positions or coordinates are relative to the beginning (5′ end) of the reference transcript.

The term “overhang” or “nucleotide overhang” herein refers to at least one unpaired nucleotide that protrudes from the end of at least one of the two strands of the duplex structure of an RNAi agent. In some embodiments, when a 3′-end of one strand extends beyond the 5′-end of the other strand, or vice versa, this forms a nucleotide overhang, e.g., the unpaired nucleotide(s) form the overhang.

“Blunt” or “blunt end” means that there are no unpaired nucleotides at that end of the double stranded RNAi agent, i.e., no nucleotide overhang. A “blunt ended” RNAi agent is a dsRNA that is double-stranded over its entire length, i.e., no nucleotide overhang at either end of the molecule.

A “mismatch” is defined herein as a difference between the base sequence (e.g., A instead of G) or length when two sequences are maximally aligned and compared. In certain aspects, the term “mismatch” means a nucleobase of a first oligonucleotide (e.g., a first strand) that is not capable of pairing with a nucleobase at a corresponding position of a second oligonucleotide (e.g., a second strand).

The term “non-end” herein refers to a position between the 3′ end and the 5′ end of the sense or antisense strand.

The term “PCSK9,” refers to a proprotein convertase subtilisin kexin 9 gene or a protein encoded by that gene. PCSK9 is also known as FH3, PC9, FHCL3, NARC1, LDLCQ1, NARC-1, or HCHOLA3. The PCSK9 gene as used herein includes human PCSK9 (e.g., Gene ID: 255738; GenBank Accession No. NM_174936.3 or NM_174936.4), mouse PCSK9 gene (e.g., Gene ID: 100102; GenBank Accession No. NM_153565.2), and dog PCSK9 gene (e.g., Gene ID: 102152231; GenBank Accession No. XM_038667514.1). Additional examples of PCSK9 mRNA sequences from different species and variants are readily available using, e.g., GenBank.

```
Homo sapiens PCSK9, transcript variant 1, mRNA: GenBank: NM_174936.3
(SEQ ID NO: 1)
      1 gtccgatggg gctctggtgg cgtgatctgc gcgccccagg cgtcaagcac ccacacccta
    61 gaaggtttcc gcagcgacgt cgaggcgctc atggttgcag gcgggcgccg ccgttcagtt
  121 cagggtctga gcctggagga gtgagccagg cagtgagact ggctcgggcg ggccgggacg
  181 cgtcgttgca gcagcggctc ccagctccca gccaggattc cgcgcgcccc ttcacgcgcc
  241 ctgctcctga acttcagctc ctgcacagtc ctccccaccg caaggctcaa ggcgccgccg
  301 gcgtggaccg cgcacggcct ctaggtctcc tcgccaggac agcaacctct cccctggccc
  361 tcatgggcac cgtcagctcc aggcggtcct ggtggccgct gccactgctg ctgctgctgc
  421 tgctgctcct gggtcccgcg ggcgcccgtg cgcaggagga cgaggacggc gactacgagg
  481 agctggtgct agccttgcgt tccgaggagg acggcctggc cgaagcaccc gagcacggaa
  541 ccacagccac cttccaccgc tgcgccaagg atccgtggag gttgcctggc acctacgtgg
  601 tggtgctgaa ggaggagacc cacctctcgc agtcagagcg cactgcccgc cgcctgcagg
  661 cccaggctgc ccgccgggga tacctcacca agatcctgca tgtcttccat ggccttcttc
  721 ctggcttcct ggtgaagatg agtggcgacc tgctggagct ggccttgaag ttgccccatg
  781 tcgactacat cgaggaggac tcctctgtct ttgcccagag catcccgtgg aacctggagc
  841 ggattacccc tccacggtac cgggcggatg aataccagcc ccccgacgga ggcagcctgg
  901 tggaggtgta tctcctagac accagcatac agagtgacca ccgggaaatc gagggcaggg
  961 tcatggtcac cgacttcgag aatgtgcccg aggaggacgg gacccgcttc cacagacagg
1021 ccagcaagtg tgacagtcat ggcacccacc tggcaggggt ggtcagcggc cgggatgccg
1081 gcgtggccaa gggtgccagc atgcgcagcc tgcgcgtgct caactgccaa gggaagggca
1141 cggttagcgg caccctcata ggcctggagt ttattcggaa aagccagctg gtccagcctg
1201 tggggccact ggtggtgctg ctgcccctgg cgggtgggta cagccgcgtc ctcaacgccg
1261 cctgccagcg cctggcgagg gctggggtcg tgctggtcac cgctgccggc aacttccggg
1321 acgatgcctg cctctactcc ccagcctcag ctcccgaggt catcacagtt ggggccacca
1381 atgcccaaga ccagccggtg accctgggga ctttggggac caactttggc cgctgtgtgg
1441 acctctttgc cccaggggag gacatcattg gtgcctccag cgactgcagc acctgctttg
1501 tgtcacagag tgggacatca caggctgctg cccacgtggc tggcattgca gccatgatgc
1561 tgtctgccga gccggagctc accctggccg agttgaggca gagactgatc cacttctctg
1621 ccaaagatgt catcaatgag gcctggttcc ctgaggacca gcgggtactg acccccaacc
1681 tggtggccgc cctgcccccc agcacccatg gggcaggttg gcagctgttt tgcaggactg
1741 tatggtcagc acactcgggg cctacacgga tggccacagc cgtcgcccgc tgcgccccag
1801 atgaggagct gctgagctgc tccagtttct ccaggagtgg gaagcggcgg ggcgagcgca
1861 tggaggccca agggggcaag ctggtctgcc gggcccacaa cgcttttggg ggtgagggtg
1921 tctacgccat tgccaggtgc tgcctgctac cccaggccaa ctgcagcgtc cacacagctc
1981 caccagctga ggccagcatg gggacccgtg tccactgcca ccaacagggc cacgtcctca
2041 caggctgcag ctcccactgg gaggtggagg accttggcac ccacaagccg cctgtgctga
2101 ggccacgagg tcagcccaac cagtgcgtgg gccacaggga ggccagcatc cacgcttcct
2161 gctgccatgc cccaggtctg gaatgcaaag tcaaggagca tggaatcccg gcccctcagg
2221 agcaggtgac cgtggcctgc gaggagggct ggaccctgac tggctgcagt gccctccctg
2281 ggacctccca cgtcctgggg gcctacgccg tagacaacac gtgtgtagtc aggagccggg
2341 acgtcagcac tacaggcagc accagcgaag gggccgtgac agccgttgcc atctgctgcc
2401 ggagccggca cctggcgcag gcctcccagg agctccagtg acagccccat cccaggatgg
2461 gtgtctgggg agggtcaagg gctggggctg agctttaaaa tggttccgac ttgtccctct
2521 ctcagccctc catggcctgg cacgagggga tggggatgct tccgcctttc cggggctgct
2581 ggcctggccc ttgagtgggg cagcctcctt gcctggaact cactcactct gggtgcctcc
2641 tccccaggtg gaggtgccag gaagctccct ccctcactgt ggggcatttc accattcaaa
2701 caggtcgagc tgtgctcggg tgctgccagc tgctcccaat gtgccgatgt ccgtgggcag
2761 aatgactttt attgagctct tgttccgtgc caggcattca atcctcaggt ctccaccaag
2821 gaggcaggat tcttcccatg gataggggag ggggcggtag gggctgcagg gacaaacatc
2881 gttggggggt gagtgtgaaa ggtgctgatg gccctcatct ccagctaact gtggagaagc
2941 ccctgggggc tccctgatta atggaggctt agctttctgg atggcatcta gccagaggct
3001 ggagacaggt gcgcccctgg tggtcacagg ctgtgccttg gtttcctgag ccacctttac
3061 tctgctctat gccaggctgt gctagcaaca cccaaaggtg gcctgcgggg agccatcacc
3121 taggactgac tcggcagtgt gcagtggtgc atgcactgtc tcagccaacc cgctccacta
3181 cccggcaggg tacacattcg cacccctact tcacagagga agaaacctgg aaccagaggg
3241 ggcgtgcctg ccaagctcac acagcaggaa ctgagccaga aacgcagatt gggctggctc
3301 tgaagccaag cctcttctta cttcacccgg ctgggctcct catttttacg ggtaacagtg
3361 aggctgggaa ggggaacaca gaccaggaag ctcggtgagt gatggcagaa cgatgcctgc
3421 aggcatggaa ctttttccgt tatcacccag gcctgattca ctggcctggc ggagatgctt
3481 ctaaggcatg gtcgggggag agggccaaca actgtccctc cttgagcacc agccccaccc
3541 aagcaagcag acatttatct tttgggtctg tcctctctgt tgccttttta cagccaactt
3601 ttctagacct gttttgcttt tgtaacttga agatatttat tctgggtttt gtagcatttt
3661 tattaatatg gtgacttttt aaaataaaaa caaacaaacg ttgtcctaac aaaaaaaaaa
3721 aaaaaaaaaa a
Homo sapiens PCSK9, transcript variant 1, mRNA: GenBank: NM_174936.4
(SEQ ID NO: 2)
      1 agcgacgtcg aggcgctcat ggttgcaggc gggcgccgcc gttcagttca gggtctgagc
    61 ctggaggagt gagccaggca gtgagactgg ctcgggcggg ccgggacgcg tcgttgcagc
  121 agcggctccc agctcccagc caggattccg cgcgcccctt cacgcgccct gctcctgaac
  181 ttcagctcct gcacagtcct ccccaccgca aggctcaagg cgccgccggc gtggaccgcg
  241 cacggcctct aggtctcctc gccaggacag caacctctcc cctggccctc atgggcaccg
  301 tcagctccag gcggtcctgg tggccgctgc cactgctgct gctgctgctg ctgctcctgg
  361 gtcccgcggg cgcccgtgcg caggaggacg aggacggcga ctacgaggag ctggtgctag
  421 ccttgcgttc cgaggaggac ggcctggccg aagcacccga gcacggaacc acagccacct
  481 tccaccgctg cgccaaggat ccgtggaggt tgcctggcac ctacgtggtg gtgctgaagg
  541 aggagaccca cctctcgcag tcagagcgca ctgcccgccg cctgcaggcc caggctgccc
  601 gccggggata cctcaccaag atcctgcatg tcttccatgg ccttcttcct ggcttcctgg
  661 tgaagatgag tggcgacctg ctggagctgg ccttgaagtt gccccatgtc gactacatcg
  721 aggaggactc ctctgtcttt gcccagagca tcccgtggaa cctggagcgg attacccctc
  781 cacggtaccg ggcggatgaa taccagcccc ccgacggagg cagcctggtg gaggtgtatc
  841 tcctagacac cagcatacag agtgaccacc gggaaatcga gggcagggtc atggtcaccg
  901 acttcgagaa tgtgcccgag gaggacggga cccgcttcca cagacaggcc agcaagtgtg
  961 acagtcatgg cacccacctg gcaggggtgg tcagcggccg ggatgccggc gtggccaagg
1021 gtgccagcat gcgcagcctg cgcgtgctca actgccaagg gaagggcacg gttagcggca
1081 ccctcatagg cctggagttt attcggaaaa gccagctggt ccagcctgtg gggccactgg
1141 tggtgctgct gcccctggcg ggtgggtaca gccgcgtcct caacgccgcc tgccagcgcc
1201 tggcgagggc tggggtcgtg ctggtcaccg ctgccggcaa cttccgggac gatgcctgcc
1261 tctactcccc agcctcagct cccgaggtca tcacagttgg ggccaccaat gcccaagacc
1321 agccggtgac cctggggact ttggggacca actttggccg ctgtgtggac ctctttgccc
1381 caggggagga catcattggt gcctccagcg actgcagcac ctgctttgtg tcacagagtg
1441 ggacatcaca ggctgctgcc cacgtggctg gcattgcagc catgatgctg tctgccgagc
1501 cggagctcac cctggccgag ttgaggcaga gactgatcca cttctctgcc aaagatgtca
1561 tcaatgaggc ctggttccct gaggaccagc gggtactgac ccccaacctg gtggccgccc
1621 tgccccccag cacccatggg gcaggttggc agctgttttg caggactgta tggtcagcac
1681 actcggggcc tacacggatg gccacagccg tcgcccgctg cgccccagat gaggagctgc
1741 tgagctgctc cagtttctcc aggagtggga agcggcgggg cgagcgcatg gaggcccaag
1801 ggggcaagct ggtctgccgg gcccacaacg cttttggggg tgagggtgtc tacgccattg
1861 ccaggtgctg cctgctaccc caggccaact gcagcgtcca cacagctcca ccagctgagg
1921 ccagcatggg gacccgtgtc cactgccacc aacagggcca cgtcctcaca ggctgcagct
1981 cccactggga ggtggaggac cttggcaccc acaagccgcc tgtgctgagg ccacgaggtc
2041 agcccaacca gtgcgtgggc cacagggagg ccagcatcca cgcttcctgc tgccatgccc
2101 caggtctgga atgcaaagtc aaggagcatg gaatcccggc ccctcaggag caggtgaccg
2161 tggcctgcga ggagggctgg accctgactg gctgcagtgc cctccctggg acctcccacg
2221 tcctgggggc ctacgccgta gacaacacgt gtgtagtcag gagccgggac gtcagcacta
2281 caggcagcac cagcgaaggg gccgtgacag ccgttgccat ctgctgccgg agccggcacc
2341 tggcgcaggc ctcccaggag ctccagtgac agccccatcc caggatgggt gtctggggag
2401 ggtcaagggc tggggctgag ctttaaaatg gttccgactt gtccctctct cagccctcca
2461 tggcctggca cgaggggatg gggatgcttc cgcctttccg gggctgctgg cctggccctt
2521 gagtggggca gcctccttgc ctggaactca ctcactctgg gtgcctcctc cccaggtgga
2581 ggtgccagga agctccctcc ctcactgtgg ggcatttcac cattcaaaca ggtcgagctg
2641 tgctcgggtg ctgccagctg ctcccaatgt gccgatgtcc gtgggcagaa tgacttttat
2701 tgagctcttg ttccgtgcca ggcattcaat cctcaggtct ccaccaagga ggcaggattc
2761 ttcccatgga taggggaggg ggcggtaggg gctgcaggga caaacatcgt tggggggtga
2821 gtgtgaaagg tgctgatggc cctcatctcc agctaactgt ggagaagccc ctgggggctc
2881 cctgattaat ggaggcttag ctttctggat ggcatctagc cagaggctgg agacaggtgc
2941 gcccctggtg gtcacaggct gtgccttggt ttcctgagcc acctttactc tgctctatgc
3001 caggctgtgc tagcaacacc caaaggtggc ctgcggggag ccatcaccta ggactgactc
3061 ggcagtgtgc agtggtgcat gcactgtctc agccaacccg ctccactacc cggcagggta
3121 cacattcgca cccctacttc acagaggaag aaacctggaa ccagaggggg cgtgcctgcc
3181 aagctcacac agcaggaact gagccagaaa cgcagattgg gctggctctg aagccaagcc
3241 tcttcttact tcacccggct gggctcctca tttttacggg taacagtgag gctgggaagg
3301 ggaacacaga ccaggaagct cggtgagtga tggcagaacg atgcctgcag gcatggaact
3361 ttttccgtta tcacccaggc ctgattcact ggcctggcgg agatgcttct aaggcatggt
3421 cgggggagag ggccaacaac tgtccctcct tgagcaccag ccccacccaa gcaagcagac
3481 atttatcttt tgggtctgtc ctctctgttg cctttttaca gccaactttt ctagacctgt
3541 tttgcttttg taacttgaag atatttattc tgggttttgt agcattttta ttaatatggt
3601 gactttttaa aataaaaaca aacaaacgtt gtcctaa
```

In certain aspects, PCSK9 may include a fragment, variant, or mutant of the protein that may have the same or similar amino acid sequences (e.g., having about 80%, 85%, 90%, 95%, or 99% or greater of similarity or identity of amino acid sequences) with any one of the above listed PCSK9 gene (mRNA) or protein sequences. In certain aspects, PCSK9 may include a fragment, variant, or mutant of the protein having the same or in similar in vivo or in vitro enzymatic (e.g., reductase) activity, for example, having about 80%, 85%, 90%, 95%, or 99% or the native protein activity, to bind to LDLR on the surface of a liver cell.

The term “Compound” as used herein refers to a double stranded RNA (e.g., PCSK9 dsRNA or dsRNAi agent) that is conjugated with a ligand or a delivery moiety, while a term “compound” denotation may refer to a substance, molecule or chemical entity that can be chemically defined and/or identifiable. In the present disclosure, Compounds are numbered in examples, e.g., “D1”

As defined herein, the term “inhibition”, “inhibit”, “inhibiting” and the like mean negatively affecting (e.g. decreasing) activity, expression or function relative to the activity, expression or function in the absence of an inhibitor. In certain aspects, inhibition can mean negatively affecting (e.g. decreasing) the concentration or levels of a biomolecule, such as a protein or mRNA, relative to the concentration or level of the biomolecule in the absence of an inhibitor. In certain aspects, inhibition includes, partially or totally, blocking stimulation, decreasing, preventing, or delaying activation; inactivating, desensitizing, or down-regulating signal transduction or enzymatic activity; or decreasing the amount of a biomolecule target (e.g., protein target or mRNA target). In certain aspects, inhibition refers to a reduction in the expression of a particular biomolecule target, such as a protein target (e.g., PCSK9 protein) or an mRNA target (e.g., PCSK9 mRNA). In certain aspects, inhibition refers to a reduction of amount of a target biomolecule (e.g., PCSK9 protein or mRNA) resulting from a down-regulating protein expression (e.g. directly inhibiting translation or transcription). In certain aspects, inhibition refers to a reduction of activity of a target biomolecule (e.g., PCSK9 protein or mRNA) from an indirect interaction (e.g., inhibiting or regulating other transcriptional or translational factors).

The term “inhibitor” also refers to a compound, composition, or substance capable of detectably negatively affecting (e.g. decreasing) activity, expression or function of a given protein or gene. For example, an inhibitor may decrease activity, expression or function by about 10%, 20%, 30%, 40%, 50%, 60%, 70%, 80%, 90% or greater in comparison to a control in the absence of the inhibitor. Inhibitors include, for example, synthetic or biological molecules, such as oligonucleotides. In some embodiments, the inhibitors include RNAi agent, e.g., siRNA agent, dsRNAi agent, or dsRNA agent.

As used herein, the “level or degree of inhibiting or decreasing expression” of a given gene refers to the at least partial suppression of the expression of a target gene (e.g., PCSK9), as manifested by a reduction of the amount of the target gene mRNA (e.g., PCSK9 mRNA) or protein (e.g., PCSK9) encoded by the target gene, which may be isolated from or detected in a group of cells (“a first cell”) in which a target gene is transcribed and which has or have been treated such that the expression of a target gene is inhibited, as compared to group of cells substantially identical to the first cell but without treated (“control cells” or “a second cell”).

In some embodiments, the level or expression of the target gene (e.g., PCSK9) can be measured by evaluation of mRNA (e.g., via Northern blots or PCR). The effect of an RNAi agent on the target gene (e.g., PCSK9) expression can be determined by measuring the gene transcription rates (e.g., via Northern blots; or reverse transcriptase polymerase chain reaction or real-time polymerase chain reaction). In some embodiments, the degree of inhibition can be calculated as the following equation:

$$\frac{( mRNA in control cells ) - ( mRNA in treated cells )}{( mRNA in control cells )} •100 \%$$

Alternatively, the degree of inhibition may be given in terms of a reduction of a parameter that is functionally linked to target gene (e.g., PCSK9) expression, e.g., the amount of protein encoded by a target gene (e.g., PCSK9), alteration in expression of the protein whose expression is dependent on the target gene (e.g., PCSK9), alteration in an activity of the enzyme (e.g., PCSK9) encoded by the target gene (e.g., PCSK9). In some embodiments, the level or expression of the protein (e.g., PCSK9) from the target gene can be evaluated by measuring the expressed protein amount (e.g., Western blots). In some embodiments, the level or expression of the protein from the target gene can be measured by the enzymatic assay (e.g., kinetic assay) of the protein.

As used herein, the term “down-regulate” or “down-regulating” refers to any decrease (e.g., statistically significant) in a biological activity and/or expression of the target protein (e.g., PCSK9), including full blocking of the activity (i.e., complete inhibition) and/or expression. For example, “down-regulation” can refer to a decrease of at least about 10%, 20%, 30%, 40%, 50%, 60%, 70%, 80%, 90% or 100% in target protein (e.g., PCSK9) level, activity and/or expression.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto. When both a basic group and an acid group are present in the same molecule, the compounds of the present invention may also form internal salts, e.g., zwitterionic molecules. In certain aspects, pharmaceutically acceptable acid addition salts can be formed with inorganic acids and organic acids. Examples of the inorganic acids from which salts can be derived include, for example, hydrochloric acid, hydrobromic acid, sulfuric acid, nitric acid, phosphoric acid, and the like. Examples of the organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. In certain aspects, the pharmaceutically acceptable base addition salts can be formed with inorganic and organic bases. Examples of the inorganic bases from which salts can be derived include, for example, ammonium salts and metals from columns I to XII of the periodic table, such as sodium, potassium, ammonium, calcium, magnesium, iron, silver, zinc, and copper; particularly suitable salts include ammonium, potassium, sodium, calcium and magnesium salts. Examples of the organic bases from which salts can be derived include, for example, primary, secondary, and tertiary amines, substituted amines including naturally occurring substituted amines, cyclic amines, basic ion exchange resins, and the like, such as organic amines include isopropylamine, benzathine, cholinate, diethanolamine, diethylamine, lysine, meglumine, piperazine and tromethamine.

In certain aspects, the term “pharmaceutically acceptable salt” as used herein may include the salts forms in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

As used herein, the term “treat,” “treating,” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient. In some embodiments, treating does not include preventing.

As used herein, the term “prevent”, “preventing” or “prevention” of any disease or disorder refers to the prophylactic treatment of the disease or disorder; or delaying the onset or progression of the disease or disorder.

The term “therapy,” as used herein refers to an application of one or more specific procedures used for the amelioration of at least one indicator or a disease or condition. In certain aspects, the specific procedure is the administration of one or more pharmaceutical or therapeutic agents.

The term “associated” or “associated with” in the context of a substance or substance activity or function associated with a disease (e.g. a protein associated disease, or PCSK9 associated disease) means that the disease is caused by (in whole or in part), or a symptom of the disease is caused by (in whole or in part) the substance or substance activity or function (e.g., PCSK9 activity or function). Thus, as used herein, what is described as “being associated” with a disease, if a causative agent, could be a target for treatment of the disease.

As used herein, the term “hyperlipidemia” refers to any disorder, disease or condition characterized by abnormal elevation of levels of any or all lipids, such as cholesterol and triglycerides, and/or lipoproteins in the blood or a condition that can lead to abnormal elevation of levels of any or all lipids and/or lipoproteins in the blood. In one embodiment, the hyperlipidemia is hypertriglyceridemia. As used herein, the term “hypertriglyceridemia” refers to a condition in which triglyceride levels are elevated, often caused or exacerbated by uncontrolled hyperlipidemia mellitus, obesity, and sedentary habits. In some embodiments, hypertriglyceridemia means triglycerides in blood are greater than 1000-2000 mg/dL. As used herein the term “hypercholesterolemia” refers to a form of hyperlipidemia (elevated levels of lipids in the blood) in which there are high levels of cholesterol in the serum of a subject. In some embodiments, hypercholesterolemia means at least about 240 mg/dL of total cholesterol.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “subject” and “patient” as used herein are used interchangeably. The term subject includes a human or non-human animal, preferably a vertebrate, and more preferably a mammal. In certain aspects, the subject is a human. In certain aspects, the subject is a human patient.

As used herein, a subject is “in need of” a treatment if such subject would benefit biologically, medically or in quality of life from such treatment.

The term “a therapeutically effective amount” of a compound (e.g., siRNA) as disclosed herein refers to an amount of the compound that will elicit the biological or medical response of a subject, for example, reduction or inhibition of an enzyme or a protein activity, or ameliorate symptoms, alleviate conditions, slow or delay disease progression, or prevent a disease, etc. In certain aspects, the term “a therapeutically effective amount” refers to the amount of the compound (e.g., siRNA) of the disclosure that, when administered to a subject, is effective to (1) at least partially alleviate, prevent and/or ameliorate a condition, or a disorder or a disease (i) mediated by the target gene (e.g., PCSK9), or (ii) associated with its activity, or (iii) characterized by activity (normal or abnormal) of the protein encoded by the target gene (e.g., PCSK9); or (2) reduce or inhibit the activity of the protein encoded by the target gene (e.g., PCSK9); or (3) reduce or inhibit the expression of the target gene (e.g., PCSK9). In certain aspects, the term “a therapeutically effective amount” refers to the amount of the compound that, when administered to a cell, or a tissue, or a non-cellular biological material, or a medium, is effective to at least partially reducing or inhibiting the activity of the protein encoded by the target gene (e.g., PCSK9); or at least partially reducing or inhibiting the expression of the protein (e.g., PCSK9) encoded by the target gene. The meaning of the term “a therapeutically effective amount” as illustrated in the above embodiment for the target gene expression also applies by the same means to any other relevant proteins/peptides/enzymes (e.g., PCSK9 or other proteins relevant to cholesterol uptake).

For any compound described herein, the therapeutically effective amount can be initially determined from cell culture assays. Target concentrations will be those concentrations of active compound(s) that are capable of achieving the methods described herein, as measured using the methods described herein or known in the art. Therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. An example of an “therapeutically effective amount” is an amount sufficient to contribute to the treatment, prevention, or reduction of a symptom or symptoms of a disease. For example, for the given parameter (e.g., biomarker), a therapeutically effective amount will show an increase or decrease of at least about 5%, about 10%, about 15%, about 20%, about 25%, about 40%, about 50%, about 60%, about 75%, about 80%, about 90%, or about 95%. Therapeutic efficacy can also be expressed as “-fold” increase or decrease. For example, a therapeutically effective amount can have at least a 1.2-fold, 1.5-fold, 2-fold, 5-fold, or more effect over a control.

The term “control” or “control experiment” is used in accordance with its plain ordinary meaning and refers to an experiment in which the subjects or reagents of the experiment are treated as in a parallel experiment except for omission of a procedure, reagent, or variable of the experiment. Typically, a control is used as a standard of comparison in evaluating experimental effects. In some embodiments, a control is the measurement of the expression of a protein or mRNA (e.g., PCSK9) in the absence of RNAi agents as described herein.

Unless defined otherwise, the chemical structures and formulae set forth herein are constructed according to the standard rules of chemical valency known in the chemical arts.

The term “alkyl,” by itself or as part of another substituent, means, unless otherwise stated, a straight (i.e., unbranched) or branched carbon chain (or carbon), or combination thereof, which is fully saturated (i.e., molecule by only single bonds) and include mono-, di- and multivalent radicals. As used herein, the alkyl is an uncyclized chain. The alkyl may include a designated number of carbons (e.g., C₁-C₁₀ means one to ten carbons). Examples of alkyl include, but are not limited to, groups such as C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl. For example, C₁₋₆ alkyl include, but are not limited to, methyl, ethyl, n-propyl, 1-methylethyl (iso-propyl), n-butyl, n-pentyl and 1,1-dimethylethyl (t-butyl), and their isomers.

A term “alkylene,” by itself or as part of another substituent, means, unless otherwise stated, a divalent radical derived from an alkyl, as exemplified, but not limited by, —CH₂CH₂CH₂CH₂—.

As used herein, the term “alkenyl,” by itself or as part of another substituent, means, unless otherwise stated, a straight (i.e., unbranched) or branched carbon chain (or carbon), or combination thereof, which is mono- or polyunsaturated (i.e., molecule including at least one double bond) and include mono-, di- and multivalent radicals. As used herein, the alkenyl is an uncyclized chain. Like the alkyl, the alkenyl may include a designated number of carbons (e.g., C₁-C₁₀ means one to ten carbons). Examples of alkenyl include, but are not limited to, groups such as C₁₋₃₀ alkenyl, C₁₋₂₅ alkenyl, C₁₋₂₀ alkenyl, C₁₋₁₅ alkenyl, C₁₋₁₂ alkenyl, C₁₋₁₀ alkenyl, C₁₋₈ alkenyl, C₁₋₆ alkenyl, C₁₋₄ alkenyl, or C₁₋₃ alkenyl. For example, C₂₋₆ alkenyl include, but are not limited to, ethenyl (vinyl), prop-1-enyl, but-1-enyl, pent-1-enyl, pent-4-enyl and penta-1,4-dienyl, and their isomers. A term “alkenylene,” by itself or as part of another substituent, means, unless otherwise stated, a divalent radical derived from an alkenyl, as exemplified, but not limited by, —CH═CHCH₂CH₂—.

As used herein, the term “alkynyl,” by itself or as part of another substituent, means, unless otherwise stated, a straight (i.e., unbranched) or branched carbon chain (or carbon), or combination thereof, which is mono- or polyunsaturated (i.e., molecule including at least one triple bond) and include mono-, di- and multivalent radicals. As used herein, the alkynyl is an uncyclized chain. Like the alkyl, the alkynyl may include a designated number of carbons (e.g., C₁-C₁₀ means one to ten carbons). Examples of alkynyl include, but are not limited to, groups such as C₁₋₃₀ alkynyl, C₁₋₂₅ alkynyl, C₁₋₂₀ alkynyl, C₁₋₁₅ alkynyl, C₁₋₁₂ alkynyl, C₁₋₁₀ alkynyl, C₁₋₈ alkynyl, C₁₋₆ alkynyl, C₁₋₄ alkynyl, or C₁₋₃ alkynyl. For example, C₂₋₆ alkynyl include, but are not limited to, alkynyl, and their isomers. A term “alkynyl,” by itself or as part of another substituent, means, unless otherwise stated, a divalent radical derived from an alkenyl, as exemplified, but not limited by, —CCH₂CH₂—.

As used herein, the term “alkoxy” refers to a radical of the formula —OR^{a} where R^{a} is an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as generally defined above. For example, C₁₋₆ alkoxy include, but are not limited to, methoxy, ethoxy, propoxy, isopropoxy, butoxy, isobutoxy, pentoxy, and hexoxy.

As used herein, the term “alkoxyalkyl” refers to a radical of the formula —R^{a}—O—R^{b} where each R^{a} and R^{b} is independently an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above and oxygen atom may be bonded to any carbon atom in either alkyl radical. For example, C₁₋₆alkoxy C₁₋₆alkyl include, but are not limited to, methoxy-methyl, methoxy-ethyl, ethoxy-ethyl, 1-ethoxy-propyl and 2-methoxy-butyl.

As used herein, the term “alkylcarbonyl” refers to a radical of the formula —C(═O)—R^{a} where R^{a} is an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above.

As used herein, the term “alkyl-carbonyl alkyl” refers to a radical of the formula, e.g., —R^{a}—C(═O)—R^{b} where each R^{a} and R^{b} is independently an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above. The carbon atom of the carbonyl group may be bonded to any carbon atom in either alkyl radical.

As used herein, the term “alkylaminocarbonyl” refers to a radical of the formula —C(═O)—NH—R^{a} where R^{a} is an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) as defined above.

As used herein, the term “alkoxycarbonyl” refers to a radical of the formula —C(═O)—O—R^{a} where R^{a} is an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above.

As used herein, the term “alkoxycarbonyl alkyl” refers to a radical of the formula —R^{a}—C(═O)—O—R^{b} where each R^{a} and R^{b} is independently an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above.

As used herein, the term “haloalkyl” refers to an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical, as defined above, substituted by one or more halo radicals, as defined above. Examples of halogenC₁₋₆alkyl include, but are not limited to, trifluoromethyl, difluoromethyl, fluoromethyl, trichloromethyl, 2,2,2-trifluoroethyl, 1,3-dibromopropan-2-yl, 3-bromo-2-fluoropropyl and 1,4,4-trifluorobutan-2-yl.

As used herein, the term “hydroxyalkyl” refers to an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above, wherein one of the hydrogen atoms of the alkyl radical is replaced by OH. Examples of hydroxyC₁₋₆ alkyl include, but are not limited to, hydroxy-methyl, 2-hydroxy-ethyl, 2-hydroxy-propyl, 3-hydroxy-propyl and 5-hydroxy-pentyl.

As used herein, the term “aminoalkyl” refers to an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above, wherein one of the hydrogen atoms of the C₁₋₆alkyl group is replaced by a primary amino group. Examples of amino C₁₋₆ alkyl include, but are not limited to, amino-methyl, 2-amino-ethyl, 2-amino-propyl, 3-amino-propyl, 3-amino-pentyl and 5-amino-pentyl.

As used herein, the term “alkylamino” refers to a radical of the formula —NH—R^{a} where R^{a} is an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above.

The term “heteroalkyl,” by itself or in combination with another term, means, unless otherwise stated, a stable straight or branched chain, or combination thereof, which is fully saturated (i.e., molecule by only single bonds) and include mono-, di- and multivalent radicals, including at least one carbon atom and at least one heteroatom (e.g., O, N, S, Si, or P), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. The heteroalkyl may include a designated number of carbons and heteroatoms (e.g., “2 to 10 membered heteroalkyl” means two to 10 atoms including carbons and heteroatoms).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like).

As used herein, the term “heteroalkenyl,” by itself or as part of another substituent, means, unless otherwise stated, a straight (i.e., unbranched) or branched carbon chain (or carbon), or combination thereof, which is mono- or polyunsaturated (i.e., molecule including at least one double bond between carbon and carbon) and include mono-, di- and multivalent radicals. As used herein, the alkenyl is an uncyclized chain. Like the alkenyl, the heteroalkenyl may include a designated number of carbons and heteroatoms (e.g., “2 to 10 membered heteroalkenyl” means two to 10 atoms including carbons and heteroatoms).

As used herein, the term “heteroalkynyl,” by itself or as part of another substituent, means, unless otherwise stated, a straight (i.e., unbranched) or branched carbon chain (or carbon), or combination thereof, which is mono- or polyunsaturated (i.e., molecule including at least one triple bond between carbon and carbon) and include mono-, di- and multivalent radicals. As used herein, the alkynyl is an uncyclized chain. The heteroalkynyl may include a designated number of carbons and heteroatoms (e.g., “2 to 10 membered heteroalkynyl” means two to 10 atoms including carbons and heteroatoms).

For alkylene and heteroalkylene linking groups, no orientation of the linking group is implied by the direction in which the formula of the linking group is written. For example, the formula —C(O)₂R′— represents both —C(O)₂R′— and —R′C(O)₂—.

A “cycloalkylene” and a “heterocycloalkylene,” alone or as part of another substituent, means a divalent radical derived from a cycloalkyl and heterocycloalkyl, respectively. The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The symbol “ [symbol] ” denotes the point of attachment of a chemical moiety to the remainder of a molecule or chemical formula.

The term “oxo,” as used herein, means an oxygen that is double-bonded to a carbon atom.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, NR′R″, SR′, -halogen, SiR′R″R′″, OC(O)R′, —C(O)R′, —CO₂R′, —CONR′R″, —OC(O)NR′R″, —NR″C(O)R′, —NR′ C(O)NR″R′″, —NR″C(O)₂R′, —NR C(NR′R″R′″)═NR′″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R′″, CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R′″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R′″ group when more than one of these groups is present. When R′ and R″ are attached to the same nitrogen atom, they can be combined with the nitrogen atom to form a 4-, 5-, 6-, or 7-membered ring. For example, NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, C(O)CF₃, C(O)CH₂OCH₃, and the like).

Certain compounds provided herein possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of provided herein do not include those that are known in art to be too unstable to synthesize and/or isolate. Compounds provided herein include those in racemic and optically pure forms. Optically active (R)- and (S)-, or (D)- and (L)-isomers may be prepared using chiral synthons or chiral reagents, or resolved using conventional techniques. When the compounds described herein contain olefinic bonds (vinyl group) and unless specified otherwise, it is intended that the compounds include both (E) and (Z) geometric isomers.

As used herein, the term “isomers” refers to compounds having the same number and kind of atoms, and hence the same molecular weight, but differing in respect to the structural arrangement or configuration of the atoms.

### RNAi Agents

In an aspect, the disclosure provides an RNAi agent including a double stranded RNA (dsRNA). In an aspect, also provided is a dsRNA interference (dsRNAi) agent that includes a dsRNA consisting of (i) a sense strand and (ii) an antisense strand, and a ligand attached to at least one of the sense strand and the antisense strand.

A dsRNA is a complex of ribonucleic acid (RNA) molecules formed in a duplex structure. In certain aspects, the dsRNA may be a small interfering RNA (siRNA) that has 10 to 30, or particularly 15-25 nucleotides in each RNA molecule, respectively, “passenger strand” and “guide strand”, and can be incorporated into an RNA-induced silencing complex (RISC). The siRNA is dissociated or unwounded in the RISC, and the passenger strand is degraded while the guide strand remains in the RISC pathway. The guide strand can subsequently bind to an mRNA molecule that includes a complementary sequence to the guide strand and induce or initiate cleavage or degradation of the mRNA molecule. In certain aspects, the mRNA encodes a target gene (e.g., mRNA transcript of a target gene) such that expression of the target gene is suppressed or inhibited through a post-transcriptional gene-silencing (“RNA silencing”). A guide RNA molecule has a complementary sequence to a target mRNA sequence and has anti-parallel orientation to the target gene, so it is interchangeably referred to as an antisense strand. A passenger RNA molecule forming a duplex with the guide RNA and having a complementary sequence to the guide strand (antisense strand) has the same orientation with the target mRNA sequence, so it is interchangeably referred to as a sense strand.

PCSK9 siRNA (Double Stranded RNA)

In an aspect, the disclosure provides a dsRNA interference (dsRNAi) agent that is capable of interacting or recruiting a target mRNA sequence, e.g., PCSK9 mRNA sequence, in the RISC thereby cleaving the target mRNA. The dsRNAi agent can silence PCSK9 gene, e.g., by inhibiting, downregulating, or suppressing the expression of PCSK9 gene. Gene-silencing (e.g., inhibiting, downregulating, or suppressing of the gene) may be assessed by a decrease in an absolute or relative level of one or more variables that are associated with PCSK9 expression compared with a control level. The control level may be any type obtained from, e.g., a pre-dose baseline level, or a level determined from a similar subject, cell, or untreated or treated subject with inactive agents (e.g., PBS buffer). In some embodiments, the level of silencing the PCSK9 may be demonstrated by a reduction of the amount of a total PCSK9 mRNA in a cell. In some embodiments, the level of silencing the PCSK9 may be demonstrated by a reduction of the amount of a total PCSK9 protein in a cell.

In some embodiments, expression of the PCSK9 gene (e.g., human PCSK9) is inhibited by at least about 10%, about 15%, about 20%, about 25%, about 30%, about 40%, about 50%, about 55%, about 60%, about 65%, about 70%, about 75%, about 80%, about 85%, about 90%, or about 95% based on the expression level of the PCSK9 gene in untreated cell or subject. In some embodiments, expression of the PCSK9 gene (e.g., human PCSK9) is inhibited by at least about 20% based on the expression level of the PCSK9 gene in untreated cell or subject. In some embodiments, expression of the PCSK9 gene (e.g., human PCSK9) is inhibited by at least about 30% based on the expression level of the PCSK9 gene in untreated cell or subject. In some embodiments, expression of the PCSK9 gene (e.g., human PCSK9) is inhibited by at least about 40% based on the expression level of the PCSK9 gene in untreated cell or subject. In some embodiments, expression of the PCSK9 gene (e.g., human PCSK9) is inhibited by at least about 50% based on the expression level of the PCSK9 gene in untreated cell or subject. In some embodiments, expression of the PCSK9 gene (e.g., human PCSK9) is inhibited by at least about 60% based on the expression level of the PCSK9 gene in untreated cell or subject. In some embodiments, expression of the PCSK9 gene (e.g., human PCSK9) is inhibited by at least about 70% based on the expression level of the PCSK9 gene in untreated cell or subject.

In some embodiments, inhibition of the expression of the PCSK9 gene may be manifested by a reduction of the amount of mRNA expressed in a first cell or a first group of cells obtained from a subject that has been treated, e.g., by contacting the cell or by administering the dsRNAi agent as described herein, as compared to a second cell or a second group of cells obtained from a subject that has not been treated but is identical to the first cell or the first group of cells. For example, the level of gene-silencing (e.g., inhibiting, downregulating, or suppressing of the gene) of the PCSK9 (e.g., human PCSK9) may be presented as a percentage of remaining mRNA in the treated cells (first cell or group of cells) compared to the mRNA amount in the control (untreated) cells, as shown in the following equation:

$$\frac{( mRNA in control cells ) - ( mRNA in treated cells )}{( mRNA in control cells )} •100 \% .$$

In some embodiments, the level of gene-silencing (e.g., inhibiting, downregulating, or suppressing of the gene) of the PCSK9 (e.g., human PCSK9) may be assessed by measuring a parameter or biomarker, e.g., human PCSK9 protein level, in a biological sample (e.g., e.g., a blood, serum or liver tissue obtained from a subject), which may be treated or untreated. Conventional analytical methods as known in the art such as electrophoresis (e.g., SDS or capillary electrophoresis), chromatography (e.g., high performance liquid chromatography (HPLC)), spectroscopy, western blotting, enzyme-linked immunosorbent assays (ELISAs), immunofluorescent assays, electrochemiluminescence assays, and the like can be used without limitation, but examples are not limited thereto. In some embodiments, reduced level of gene-silencing (e.g., inhibiting, downregulating, or suppressing of the gene) of the PCSK9 (e.g., human PCSK9) may be observed or assessed by in a liver (tissue) biopsy of the treated subject.

In certain aspects, the dsRNAi agent is a free acid. In certain aspects, the dsRNAi agent is in a salt form (e.g., a pharmaceutically acceptable salt form). It will be understood that references to dsRNAi agent are meant to also include the pharmaceutically acceptable salts of the dsRNAi agent. If the dsRNAi agent has, for example, at least one basic center, they can form acid addition salts. Corresponding acid addition salts can also be formed having, if desired, an additionally present basic center. Active substances having an acid group, e.g., COOH, can form salts with bases. The dsRNAi agent or pharmaceutically acceptable salts thereof may also be used in form of a hydrate or include other solvents used for crystallization. In some embodiments, the RNAi agent is a sodium salt. In some embodiments, the dsRNAi agent is in a salt form (e.g., a pharmaceutically acceptable salt form), where the salt is sodium (Na⁺), ammonium (NH₄⁺), calcium (Ca²⁺), iron (Fe²⁺ or Fe³⁺), magnesium (Mg²⁺), potassium (K⁺), pyridinium (C₅H₅NH⁺), quaternary ammonium (NR₄⁺, R being an alkyl group or an aryl group as described herein), or copper (Cu²⁺).

In an aspect, the disclosure provides a dsRNA having sequences (e.g., antisense strand sequence) that can recognize a specific region of a PCSK9 mRNA (e.g., human PCSK9 mRNA) and lead cleavage of the PCSK9 mRNA and silencing of the gene. The dsRNA includes a sense strand and an antisense strand and each strand may range from 12 to 30 nucleotides in length. In some embodiments, each strand may have 15 to 30 nucleotides in length. In some embodiments, each strand may have 15 to 25 nucleotides in length. In some embodiments, the antisense strand may have 15 to 25 nucleotides in length. In some embodiments, the sense strand may have 15 to 25 nucleotides in length. In some embodiments, the antisense strand may have 15 to 23 nucleotides in length. In some embodiments, the sense strand may have 15 to 23 nucleotides in length. In some embodiments, the antisense strand may have 18 to 25 nucleotides in length. In some embodiments, the sense strand may have 18 to 25 nucleotides in length.

In some embodiments, the sense strand may have 19 to 23 nucleotides in length. In some embodiments, the sense strand may have 21 to 23 nucleotides in length. In some embodiments, the sense strand may have 19 nucleotides in length. In some embodiments, the sense strand may have 20 nucleotides in length. In some embodiments, the sense strand may have 21 nucleotides in length. In some embodiments, the sense strand may have 22 nucleotides in length. In some embodiments, the sense strand may have 23 nucleotides in length.

In some embodiments, the antisense strand may have 19 to 25 nucleotides in length. In some embodiments, the antisense strand may have 19 to 23 nucleotides in length. In some embodiments, the antisense strand may have 21 to 23 nucleotides in length. In some embodiments, the antisense strand may have 23 to 25 nucleotides in length. In some embodiments, the antisense strand may have 19 nucleotides in length. In some embodiments, the antisense strand may have 20 nucleotides in length. In some embodiments, the antisense strand may have 21 nucleotides in length. In some embodiments, the antisense strand may have 22 nucleotides in length. In some embodiments, the antisense strand may have 23 nucleotides in length. In some embodiments, the antisense strand may have 24 nucleotides in length. In some embodiments, the antisense strand may have 25 nucleotides in length.

In some embodiments, the sense strand is 21 to 23 nucleotides in length and the antisense strand is 23 to 25 nucleotides in length. In some embodiments, the sense strand is 21 nucleotides in length and the antisense strand is 23 nucleotides in length. In some embodiments, the sense strand is 22 nucleotides in length and the antisense strand is 24 nucleotides in length. In some embodiments, the sense strand is 23 nucleotides in length and the antisense strand is 25 nucleotides in length.

In an aspect, a dsRNA as described herein forms a double-stranded (or “duplex”) region made between a sense strand and an antisense strand and having 10 to 25 nucleotide pairs in length. The double stranded or duplex region are loaded into the RISC and subsequent specific degradation of the sense strand occurs during the RISC pathway. In some embodiments, the double stranded region has 10 nucleotide base pairs in length. In some embodiments, the double stranded region has 11 nucleotide base pairs in length. In some embodiments, the double stranded region has 12 nucleotide base pairs in length. In some embodiments, the double stranded region has 13 nucleotide base pairs in length. In some embodiments, the double stranded region has 14 nucleotide base pairs in length. In some embodiments, the double stranded region has 15 nucleotide base pairs in length. In some embodiments, the double stranded region has 16 nucleotide base pairs in length. In some embodiments, the double stranded region has 17 nucleotide base pairs in length. In some embodiments, the double stranded region has 18 nucleotide base pairs in length. In some embodiments, the double stranded region has 19 nucleotide base pairs in length. In some embodiments, the double stranded region has 20 nucleotide base pairs in length. In some embodiments, the double stranded region has 21 nucleotide base pairs in length. In some embodiments, the double stranded region has 22 nucleotide base pairs in length. In some embodiments, the double stranded region has 23 nucleotide base pairs in length.

In an aspect, a dsRNA as described herein may include at least one single-stranded nucleotide overhang, for example, for increasing in vivo effectiveness of the dsRNA and having substantially improved inhibition of the target genes. In certain aspects, the dsRNA may contain one or more extra nucleotides constituting overhang regions that locate other than the double stranded region at the 3′-end, 5′-end, or both ends of either stand or both strands (sense and antisense strands). In some embodiments, the overhang region may exist at the 3′-end, 5′-end, or both ends of the sense strand. In some embodiments, the overhang region may exist at the 3′-end, 5′-end, or both ends of the antisense strand. In some embodiments, the antisense strand may have a greater length than a length in the sense strand. In some embodiments, the antisense strand may have a shorter length than a length in the sense strand.

In some embodiments, the dsRNA may contain one or more extra nucleotides constituting overhang regions at the 3′-end, 5′-end, or both ends of the antisense strand. In some embodiments, the overhang region in the antisense strand may consist of 1-6 nucleotides in length, for example, 1 nucleotide, 2 nucleotides, 3 nucleotides, 4 nucleotides, 5 nucleotides, or 6 nucleotides in length. In some embodiments, the dsRNA may contain one or more extra nucleotides constituting overhang regions at the 3′-end, 5′-end, or both ends of the sense strand. In some embodiments, the overhang region may consist of 1 to 6 nucleotides in length, for example, 1 nucleotide, 2 nucleotides, 3 nucleotides, 4 nucleotides, 5 nucleotides, or 6 nucleotides in length.

In some embodiments, the antisense strand may include one-nucleotide overhang at the 5′ end. In some embodiments, the antisense strand may include one-nucleotide overhang at the 3′ end. In some embodiments, the antisense strand may include two-nucleotides overhang. In some embodiments, the antisense contains two-nucleotides overhang at the 5′ end. In some embodiments, the antisense contains two-nucleotides overhang at the 3′ end. In some embodiments, the antisense contains one-nucleotide overhang at the 5′ end and one-nucleotide overhang at the 3′ end. In some embodiments, the antisense strand may include three-nucleotide overhang. In some embodiments, the antisense contains three-nucleotides overhang at the 5′ end. In some embodiments, the antisense contains three-nucleotides overhang at the 3′ end. In some embodiments, the antisense contains two-nucleotides overhang at the 5′ end and one-nucleotide overhang at the 3′ end. In some embodiments, the antisense contains two nucleotides overhang at the 3′ end and one-nucleotide overhang at the 5′ end.

In certain aspects, a dsRNA as described herein may include at least one blunt end, e.g., for increasing in vivo stability with resistance to degradation in physiological surroundings. In some embodiments, the dsRNA may have a blunt end at the 3′-end, 5′-end, or both ends of the duplex. In some embodiments, the dsRNA includes one overhang (e.g., at 3′ end of antisense strand) and one blunt end (e.g., at 5′ end of antisense strand). In some embodiments, the dsRNA includes a blunt end at the 5′-end of the sense strand (and at 3′ end of the antisense strand) and contain overhang nucleotide(s) at the other end. In some embodiments, the dsRNA may have a blunt end at the 3′-end of the sense strand (and at 5′ end of the antisense strand) and contain overhang nucleotide(s) at the other end.

In certain aspects, the target PCSK9 mRNA sequence may range from 12 to 30 nucleotides, from 15 to 30 nucleotides, from 18 to 30 nucleotides, from 18 to 25 nucleotides, from 18 to 23 nucleotides. In certain aspects, the target PCSK9 mRNA sequence may range from 19 to 25 nucleotides, from 19 to 23 nucleotides, or from 19 to 21 nucleotides. In some embodiments, the target PCSK9 mRNA sequence may have 15 nucleotides in length. In some embodiments, the target PCSK9 mRNA sequence may have 16 nucleotides in length. In some embodiments, the target PCSK9 mRNA sequence may have 17 nucleotides in length. In some embodiments, the target PCSK9 mRNA sequence may have 18 nucleotides in length. In some embodiments, the target PCSK9 mRNA sequence may have 19 nucleotides in length. In some embodiments, the target PCSK9 mRNA sequence may have 20 nucleotides in length. In some embodiments, the target PCSK9 mRNA sequence may have 21 nucleotides in length. In some embodiments, the target PCSK9 mRNA sequence may have 22 nucleotides in length. In some embodiments, the target PCSK9 mRNA sequence may have 23 nucleotides in length.

In certain aspects, exemplary dsRNA sequences including sense strands and antisense strands targeting human PCSK9 mRNAs of SEQ ID NO: 2 (GenBank: NM_174936.4) are in Table 1.

TABLE 1
Examples of unmodified nucleotide sequences of PCSK9 siRNA

| | Site | | | | | |
|---|---|---|---|---|---|---|
| | of | | | | | |
| | mRNA | | | SEQ | | SEQ |
| PCSK9 | Tar- | | | ID | | ID |
| SiRNA | get* | Region | Sense sequence | NO: | Antisense sequence | NO: |
| U1 | 27 | 5′ | GUCGAGGCGCUCAUG | 3 | UGCAACCAUGAGCGC | 382 |
| | | UTR | GUUGCA | | CUCGACGU | |
| U2 | 30 | 5′ | GAGGCGCUCAUGGUU | 4 | UCCUGCAACCAUGAG | 383 |
| | | UTR | GCAGGA | | CGCCUCGA | |
| U3 | 117 | 5′ | CGGGCCGGGACGCGU | 5 | UCAACGACGCGUCCC | 384 |
| | | UTR | CGUUGA | | GGCCCGCC | |
| U4 | 121 | 5′ | CCGGGACGCGUCGUU | 6 | UGCUGCAACGACGCG | 385 |
| | | UTR | GCAGCA | | UCCCGGCC | |
| U5 | 153 | 5′ | CUCCCAGCCAGGAUU | 7 | UCGCGGAAUCCUGGC | 386 |
| | | UTR | CCGCGA | | UGGGAGCU | |
| U6 | 155 | 5′ | CCCAGCCAGGAUUCC | 8 | UCGCGCGGAAUCCUG | 387 |
| | | UTR | GCGCGA | | GCUGGGAG | |
| U7 | 161 | 5′ | CAGGAUUCCGCGCGC | 9 | UAAGGGGCGCGCGGA | 388 |
| | | UTR | CCCUUA | | AUCCUGGC | |
| U8 | 219 | 5′ | CUCCCCACCGCAAGG | 10 | UUUGAGCCUUGCGGU | 389 |
| | | UTR | CUCAAA | | GGGGAGGA | |
| U9 | 220 | 5′ | UCCCCACCGCAAGGC | 11 | UCUUGAGCCUUGCGG | 390 |
| | | UTR | UCAAGG | | UGGGGAGG | |
| U10 | 256 | 5′ | CCGCGCACGGCCUCU | 12 | AGACCUAGAGGCCGU | 391 |
| | | UTR | AGGUCU | | GCGCGGUC | |
| U11 | 256 | 5′ | CCGCGCACGGCCUCU | 13 | UGACCUAGAGGCCGU | 392 |
| | | UTR | AGGUCA | | GCGCGGUC | |
| U12 | 257 | 5′ | CGCGCACGGCCUCUA | 14 | UAGACCUAGAGGCCG | 393 |
| | | UTR | GGUCUA | | UGCGCGGU | |
| U13 | 260 | 5′ | GCACGGCCUCUAGGU | 15 | UAGGAGACCUAGAGG | 394 |
| | | UTR | CUCCUA | | CCGUGCGC | |
| U14 | 316 | CDS | CACCGUCAGCUCCAG | 16 | UACCGCCUGGAGCUG | 395 |
| | | | GCGGUA | | ACGGUGCC | |
| U15 | 317 | CDS | ACCGUCAGCUCCAGG | 17 | UGACCGCCUGGAGCU | 396 |
| | | | CGGUCC | | GACGGUGC | |
| U16 | 318 | CDS | CCGUCAGCUCCAGGC | 18 | AGGACCGCCUGGAGC | 397 |
| | | | GGUCCU | | UGACGGUG | |
| U17 | 318 | CDS | CCGUCAGCUCCAGGC | 19 | UGGACCGCCUGGAGC | 398 |
| | | | GGUCCA | | UGACGGUG | |
| U18 | 378 | CDS | UGGGUCCCGCGGGCG | 20 | UACGGGCGCCCGCGG | 399 |
| | | | CCCGUG | | GACCCAGG | |
| U19 | 386 | CDS | GCGGGCGCCCGUGCG | 21 | UUCCUGCGCACGGGC | 400 |
| | | | CAGGAA | | GCCCGCGG | |
| U20 | 392 | CDS | GCCCGUGCGCAGGAG | 22 | UUCGUCCUCCUGCGC | 401 |
| | | | GACGAA | | ACGGGCGC | |
| U21 | 393 | CDS | CCCGUGCGCAGGAGG | 23 | UCUCGUCCUCCUGCG | 402 |
| | | | ACGAGA | | CACGGGCG | |
| U22 | 404 | CDS | GAGGACGAGGACGGC | 24 | UUAGUCGCCGUCCUC | 403 |
| | | | GACUAA | | GUCCUCCU | |
| U23 | 421 | CDS | CUACGAGGAGCUGGU | 25 | UCUAGCACCAGCUCC | 404 |
| | | | GCUAGA | | UCGUAGUC | |
| U24 | 425 | CDS | GAGGAGCUGGUGCUA | 26 | UAAGGCUAGCACCAG | 405 |
| | | | GCCUUA | | CUCCUCGU | |
| U25 | 430 | CDS | GCUGGUGCUAGCCUU | 27 | UAACGCAAGGCUAGC | 406 |
| | | | GCGUUA | | ACCAGCUC | |
| U26 | 432 | CDS | UGGUGCUAGCCUUGC | 28 | UGGAACGCAAGGCUA | 407 |
| | | | GUUCCG | | GCACCAGC | |
| U27 | 437 | CDS | CUAGCCUUGCGUUCC | 29 | UUCCUCGGAACGCAA | 408 |
| | | | GAGGAA | | GGCUAGCA | |
| U28 | 476 | CDS | CCCGAGCACGGAACC | 30 | UGCUGUGGUUCCGUG | 409 |
| | | | ACAGCA | | CUCGGGUG | |
| U29 | 479 | CDS | GAGCACGGAACCACA | 31 | UGUGGCUGUGGUUCC | 410 |
| | | | GCCACA | | GUGCUCGG | |
| U30 | 490 | CDS | CACAGCCACCUUCCA | 32 | UAGCGGUGGAAGGUG | 411 |
| | | | CCGCUA | | GCUGUGGU | |
| U31 | 496 | CDS | CACCUUCCACCGCUG | 33 | UUGGCGCAGCGGUGG | 412 |
| | | | CGCCAA | | AAGGUGGC | |
| U32 | 497 | CDS | ACCUUCCACCGCUGC | 34 | UUUGGCGCAGCGGUG | 413 |
| | | | GCCAAG | | GAAGGUGG | |
| U33 | 500 | CDS | UUCCACCGCUGCGCC | 35 | AUCCUUGGCGCAGCG | 414 |
| | | | AAGGAU | | GUGGAAGG | |
| U34 | 500 | CDS | UUCCACCGCUGCGCC | 36 | UUCCUUGGCGCAGCG | 415 |
| | | | AAGGAA | | GUGGAAGG | |
| U35 | 502 | CDS | CCACCGCUGCGCCAA | 37 | UGAUCCUUGGCGCAG | 416 |
| | | | GGAUCA | | CGGUGGAA | |
| U36 | 503 | CDS | CACCGCUGCGCCAAG | 38 | UGGAUCCUUGGCGCA | 417 |
| | | | GAUCCA | | GCGGUGGA | |
| U37 | 505 | CDS | CCGCUGCGCCAAGGA | 39 | UACGGAUCCUUGGCG | 418 |
| | | | UCCGUA | | CAGCGGUG | |
| U38 | 506 | CDS | CGCUGCGCCAAGGAU | 40 | UCACGGAUCCUUGGC | 419 |
| | | | CCGUGA | | GCAGCGGU | |
| U39 | 539 | CDS | ACCUACGUGGUGGUG | 41 | UUUCAGCACCACCAC | 420 |
| | | | CUGAAG | | GUAGGUGC | |
| U40 | 570 | CDS | ACCUCUCGCAGUCAG | 42 | UGCGCUCUGACUGCG | 421 |
| | | | AGCGCA | | AGAGGUGG | |
| U41 | 571 | CDS | CCUCUCGCAGUCAGA | 43 | UUGCGCUCUGACUGC | 422 |
| | | | GCGCAA | | GAGAGGUG | |
| U42 | 573 | CDS | UCUCGCAGUCAGAGC | 44 | UAGUGCGCUCUGACU | 423 |
| | | | GCACUG | | GCGAGAGG | |
| U43 | 611 | CDS | CAGGCUGCCCGCCGG | 45 | UUAUCCCCGGCGGGC | 424 |
| | | | GGAUAA | | AGCCUGGG | |
| U44 | 613 | CDS | GGCUGCCCGCCGGGG | 46 | AGGUAUCCCCGGCGG | 425 |
| | | | AUACCU | | GCAGCCUG | |
| U45 | 613 | CDS | GGCUGCCCGCCGGGG | 47 | UGGUAUCCCCGGCGG | 426 |
| | | | AUACCA | | GCAGCCUG | |
| U46 | 616 | CDS | UGCCCGCCGGGGAUA | 48 | UUGAGGUAUCCCCGG | 427 |
| | | | CCUCAC | | CGGGCAGC | |
| U47 | 620 | CDS | CGCCGGGGAUACCUC | 49 | UUUGGUGAGGUAUCC | 428 |
| | | | ACCAAA | | CCGGCGGG | |
| U48 | 621 | CDS | GCCGGGGAUACCUCA | 50 | UCUUGGUGAGGUAUC | 429 |
| | | | CCAAGA | | CCCGGCGG | |
| U49 | 623 | CDS | CGGGGAUACCUCACC | 51 | UAUCUUGGUGAGGUA | 430 |
| | | | AAGAUA | | UCCCCGGC | |
| U50 | 624 | CDS | GGGGAUACCUCACCA | 52 | UGAUCUUGGUGAGGU | 431 |
| | | | AGAUCA | | AUCCCCGG | |
| U51 | 668 | CDS | CCUGGCUUCCUGGUG | 53 | UAUCUUCACCAGGAA | 432 |
| | | | AAGAUA | | GCCAGGAA | |
| U52 | 680 | CDS | GUGAAGAUGAGUGGC | 54 | UAGGUCGCCACUCAU | 433 |
| | | | GACCUA | | CUUCACCA | |
| U53 | 683 | CDS | AAGAUGAGUGGCGAC | 55 | UAGCAGGUCGCCACU | 434 |
| | | | CUGCUG | | CAUCUUCA | |
| U54 | 709 | CDS | GGCCUUGAAGUUGCC | 56 | ACAUGGGGCAACUUC | 435 |
| | | | CCAUGU | | AAGGCCAG | |
| U55 | 709 | CDS | GGCCUUGAAGUUGCC | 57 | UCAUGGGGCAACUUC | 436 |
| | | | CCAUGA | | AAGGCCAG | |
| U56 | 711 | CDS | CCUUGAAGUUGCCCC | 58 | UGACAUGGGGCAACU | 437 |
| | | | AUGUCA | | UCAAGGCC | |
| U57 | 712 | CDS | CUUGAAGUUGCCCCA | 59 | UCGACAUGGGGCAAC | 438 |
| | | | UGUCGA | | UUCAAGGC | |
| U58 | 713 | CDS | UUGAAGUUGCCCCAU | 60 | UUCGACAUGGGGCAA | 439 |
| | | | GUCGAC | | CUUCAAGG | |
| U59 | 714 | CDS | UGAAGUUGCCCCAUG | 61 | AGUCGACAUGGGGCA | 440 |
| | | | UCGACU | | ACUUCAAG | |
| U60 | 714 | CDS | UGAAGUUGCCCCAUG | 62 | UGUCGACAUGGGGCA | 441 |
| | | | UCGACA | | ACUUCAAG | |
| U61 | 715 | CDS | GAAGUUGCCCCAUGU | 63 | UAGUCGACAUGGGGC | 442 |
| | | | CGACUA | | AACUUCAA | |
| U62 | 716 | CDS | AAGUUGCCCCAUGUC | 64 | UUAGUCGACAUGGGG | 443 |
| | | | GACUAC | | CAACUUCA | |
| U63 | 717 | CDS | AGUUGCCCCAUGUCG | 65 | UGUAGUCGACAUGGG | 444 |
| | | | ACUACA | | GCAACUUC | |
| U64 | 718 | CDS | GUUGCCCCAUGUCGA | 66 | AUGUAGUCGACAUGG | 445 |
| | | | CUACAU | | GGCAACUU | |
| U65 | 718 | CDS | GUUGCCCCAUGUCGA | 67 | UUGUAGUCGACAUGG | 446 |
| | | | CUACAA | | GGCAACUU | |
| U66 | 720 | CDS | UGCCCCAUGUCGACU | 68 | UGAUGUAGUCGACAU | 447 |
| | | | ACAUCG | | GGGGCAAC | |
| U67 | 721 | CDS | GCCCCAUGUCGACUA | 69 | UCGAUGUAGUCGACA | 448 |
| | | | CAUCGA | | UGGGGCAA | |
| U68 | 722 | CDS | CCCCAUGUCGACUAC | 70 | UUCGAUGUAGUCGAC | 449 |
| | | | AUCGAA | | AUGGGGCA | |
| U69 | 725 | CDS | CAUGUCGACUACAUC | 71 | UUCCUCGAUGUAGUC | 450 |
| | | | GAGGAA | | GACAUGGG | |
| U70 | 726 | CDS | AUGUCGACUACAUCG | 72 | UCUCCUCGAUGUAGU | 451 |
| | | | AGGAGG | | CGACAUGG | |
| U71 | 728 | CDS | GUCGACUACAUCGAG | 73 | UUCCUCCUCGAUGUA | 452 |
| | | | GAGGAA | | GUCGACAU | |
| U72 | 731 | CDS | GACUACAUCGAGGAG | 74 | UGAGUCCUCCUCGAU | 453 |
| | | | GACUCA | | GUAGUCGA | |
| U73 | 770 | CDS | AUCCCGUGGAACCUG | 75 | UCGCUCCAGGUUCCA | 454 |
| | | | GAGCGG | | CGGGAUGC | |
| U74 | 794 | CDS | ACCCCUCCACGGUAC | 76 | UGCCCGGUACCGUGG | 455 |
| | | | CGGGCG | | AGGGGUAA | |
| U75 | 795 | CDS | CCCCUCCACGGUACC | 77 | UCGCCCGGUACCGUG | 456 |
| | | | GGGCGA | | GAGGGGUA | |
| U76 | 796 | CDS | CCCUCCACGGUACCG | 78 | UCCGCCCGGUACCGU | 457 |
| | | | GGCGGA | | GGAGGGGU | |
| U77 | 798 | CDS | CUCCACGGUACCGGG | 79 | UAUCCGCCCGGUACC | 458 |
| | | | CGGAUA | | GUGGAGGG | |
| U78 | 799 | CDS | UCCACGGUACCGGGC | 80 | UCAUCCGCCCGGUAC | 459 |
| | | | GGAUGA | | CGUGGAGG | |
| U79 | 800 | CDS | CCACGGUACCGGGCG | 81 | UUCAUCCGCCCGGUA | 460 |
| | | | GAUGAA | | CCGUGGAG | |
| U80 | 801 | CDS | CACGGUACCGGGCGG | 82 | AUUCAUCCGCCCGGU | 461 |
| | | | AUGAAU | | ACCGUGGA | |
| U81 | 801 | CDS | CACGGUACCGGGCGG | 83 | UUUCAUCCGCCCGGU | 462 |
| | | | AUGAAA | | ACCGUGGA | |
| U82 | 802 | CDS | ACGGUACCGGGCGGA | 84 | UAUUCAUCCGCCCGG | 463 |
| | | | UGAAUA | | UACCGUGG | |
| U83 | 806 | CDS | UACCGGGCGGAUGAA | 85 | UUGGUAUUCAUCCGC | 464 |
| | | | UACCAG | | CCGGUACC | |
| U84 | 840 | CDS | GCAGCCUGGUGGAGG | 86 | UAUACACCUCCACCA | 465 |
| | | | UGUAUA | | GGCUGCCU | |
| U85 | 843 | CDS | GCCUGGUGGAGGUGU | 87 | UGAGAUACACCUCCA | 466 |
| | | | AUCUCA | | CCAGGCUG | |
| U86 | 847 | CDS | GGUGGAGGUGUAUCU | 88 | UCUAGGAGAUACACC | 467 |
| | | | CCUAGA | | UCCACCAG | |
| U87 | 848 | CDS | GUGGAGGUGUAUCUC | 89 | UUCUAGGAGAUACAC | 468 |
| | | | CUAGAA | | CUCCACCA | |
| U88 | 850 | CDS | GGAGGUGUAUCUCCU | 90 | UUGUCUAGGAGAUAC | 469 |
| | | | AGACAA | | ACCUCCAC | |
| U89 | 851 | CDS | GAGGUGUAUCUCCUA | 91 | UGUGUCUAGGAGAUA | 470 |
| | | | GACACA | | CACCUCCA | |
| U90 | 852 | CDS | AGGUGUAUCUCCUAG | 92 | UGGUGUCUAGGAGAU | 471 |
| | | | ACACCA | | ACACCUCC | |
| U91 | 853 | CDS | GGUGUAUCUCCUAGA | 93 | UUGGUGUCUAGGAGA | 472 |
| | | | CACCAA | | UACACCUC | |
| U92 | 856 | CDS | GUAUCUCCUAGACAC | 94 | AUGCUGGUGUCUAGG | 473 |
| | | | CAGCAU | | AGAUACAC | |
| U93 | 856 | CDS | GUAUCUCCUAGACAC | 95 | UUGCUGGUGUCUAGG | 474 |
| | | | CAGCAA | | AGAUACAC | |
| U94 | 857 | CDS | UAUCUCCUAGACACC | 96 | UAUGCUGGUGUCUAG | 475 |
| | | | AGCAUA | | GAGAUACA | |
| U95 | 858 | CDS | AUCUCCUAGACACCA | 97 | UUAUGCUGGUGUCUA | 476 |
| | | | GCAUAC | | GGAGAUAC | |
| U96 | 862 | CDS | CCUAGACACCAGCAU | 98 | UUCUGUAUGCUGGUG | 477 |
| | | | ACAGAA | | UCUAGGAG | |
| U97 | 868 | CDS | CACCAGCAUACAGAG | 99 | UGGUCACUCUGUAUG | 478 |
| | | | UGACCA | | CUGGUGUC | |
| U98 | 870 | CDS | CCAGCAUACAGAGUG | 100 | UGUGGUCACUCUGUA | 479 |
| | | | ACCACA | | UGCUGGUG | |
| U99 | 873 | CDS | GCAUACAGAGUGACC | 101 | UCCGGUGGUCACUCU | 480 |
| | | | ACCGGA | | GUAUGCUG | |
| U100 | 875 | CDS | AUACAGAGUGACCAC | 102 | UUCCCGGUGGUCACU | 481 |
| | | | CGGGAA | | CUGUAUGC | |
| U101 | 876 | CDS | UACAGAGUGACCACC | 103 | UUUCCCGGUGGUCAC | 482 |
| | | | GGGAAA | | UCUGUAUG | |
| U102 | 878 | CDS | CAGAGUGACCACCGG | 104 | UAUUUCCCGGUGGUC | 483 |
| | | | GAAAUA | | ACUCUGUA | |
| U103 | 879 | CDS | AGAGUGACCACCGGG | 105 | UGAUUUCCCGGUGGU | 484 |
| | | | AAAUCG | | CACUCUGU | |
| U104 | 880 | CDS | GAGUGACCACCGGGA | 106 | UCGAUUUCCCGGUGG | 485 |
| | | | AAUCGA | | UCACUCUG | |
| U105 | 881 | CDS | AGUGACCACCGGGAA | 107 | UUCGAUUUCCCGGUG | 486 |
| | | | AUCGAG | | GUCACUCU | |
| U106 | 882 | CDS | GUGACCACCGGGAAA | 108 | UCUCGAUUUCCCGGU | 487 |
| | | | UCGAGA | | GGUCACUC | |
| U107 | 883 | CDS | UGACCACCGGGAAAU | 109 | UCCUCGAUUUCCCGG | 488 |
| | | | CGAGGG | | UGGUCACU | |
| U108 | 892 | CDS | GGAAAUCGAGGGCAG | 110 | AUGACCCUGCCCUCG | 489 |
| | | | GGUCAU | | AUUUCCCG | |
| U109 | 892 | CDS | GGAAAUCGAGGGCAG | 111 | UUGACCCUGCCCUCG | 490 |
| | | | GGUCAA | | AUUUCCCG | |
| U110 | 893 | CDS | GAAAUCGAGGGCAGG | 112 | UAUGACCCUGCCCUC | 491 |
| | | | GUCAUA | | GAUUUCCC | |
| U111 | 894 | CDS | AAAUCGAGGGCAGGG | 113 | UCAUGACCCUGCCCU | 492 |
| | | | UCAUGG | | CGAUUUCC | |
| U112 | 896 | CDS | AUCGAGGGCAGGGUC | 114 | UACCAUGACCCUGCC | 493 |
| | | | AUGGUC | | CUCGAUUU | |
| U113 | 900 | CDS | AGGGCAGGGUCAUGG | 115 | UGGUGACCAUGACCC | 494 |
| | | | UCACCG | | UGCCCUCG | |
| U114 | 902 | CDS | GGCAGGGUCAUGGUC | 116 | UUCGGUGACCAUGAC | 495 |
| | | | ACCGAA | | CCUGCCCU | |
| U115 | 903 | CDS | GCAGGGUCAUGGUCA | 117 | AGUCGGUGACCAUGA | 496 |
| | | | CCGACU | | CCCUGCCC | |
| U116 | 903 | CDS | GCAGGGUCAUGGUCA | 118 | UGUCGGUGACCAUGA | 497 |
| | | | CCGACA | | CCCUGCCC | |
| U117 | 904 | CDS | CAGGGUCAUGGUCAC | 119 | AAGUCGGUGACCAUG | 498 |
| | | | CGACUU | | ACCCUGCC | |
| U118 | 904 | CDS | CAGGGUCAUGGUCAC | 120 | UAGUCGGUGACCAUG | 499 |
| | | | CGACUA | | ACCCUGCC | |
| U119 | 945 | CDS | ACGGGACCCGCUUCC | 121 | UUCUGUGGAAGCGGG | 500 |
| | | | ACAGAC | | UCCCGUCC | |
| U120 | 946 | CDS | CGGGACCCGCUUCCA | 122 | UGUCUGUGGAAGCGG | 501 |
| | | | CAGACA | | GUCCCGUC | |
| U121 | 960 | CDS | ACAGACAGGCCAGCA | 123 | UACACUUGCUGGCCU | 502 |
| | | | AGUGUG | | GUCUGUGG | |
| U122 | 976 | CDS | GUGUGACAGUCAUGG | 124 | UGGGUGCCAUGACUG | 503 |
| | | | CACCCA | | UCACACUU | |
| U123 | 993 | CDS | CCCACCUGGCAGGGG | 125 | UGACCACCCCUGCCA | 504 |
| | | | UGGUCA | | GGUGGGUG | |
| U124 | 994 | CDS | CCACCUGGCAGGGGU | 126 | UUGACCACCCCUGCC | 505 |
| | | | GGUCAA | | AGGUGGGU | |
| U125 | 1054 | CDS | CAGCCUGCGCGUGCU | 127 | UAGUUGAGCACGCGC | 506 |
| | | | CAACUA | | AGGCUGCG | |
| U126 | 1055 | CDS | AGCCUGCGCGUGCUC | 128 | UCAGUUGAGCACGCG | 507 |
| | | | AACUGC | | CAGGCUGC | |
| U127 | 1059 | CDS | UGCGCGUGCUCAACU | 129 | UUUGGCAGUUGAGCA | 508 |
| | | | GCCAAG | | CGCGCAGG | |
| U128 | 1060 | CDS | GCGCGUGCUCAACUG | 130 | UCUUGGCAGUUGAGC | 509 |
| | | | CCAAGA | | ACGCGCAG | |
| U129 | 1070 | CDS | AACUGCCAAGGGAAG | 131 | UGUGCCCUUCCCUUG | 510 |
| | | | GGCACG | | GCAGUUGA | |
| U130 | 1071 | CDS | ACUGCCAAGGGAAGG | 132 | UCGUGCCCUUCCCUU | 511 |
| | | | GCACGG | | GGCAGUUG | |
| U131 | 1081 | CDS | GAAGGGCACGGUUAG | 133 | UUGCCGCUAACCGUG | 512 |
| | | | CGGCAA | | CCCUUCCC | |
| U132 | 1082 | CDS | AAGGGCACGGUUAGC | 134 | UGUGCCGCUAACCGU | 513 |
| | | | GGCACC | | GCCCUUCC | |
| U133 | 1085 | CDS | GGCACGGUUAGCGGC | 135 | UAGGGUGCCGCUAAC | 514 |
| | | | ACCCUA | | CGUGCCCU | |
| U134 | 1088 | CDS | ACGGUUAGCGGCACC | 136 | UAUGAGGGUGCCGCU | 515 |
| | | | CUCAUA | | AACCGUGC | |
| U135 | 1089 | CDS | CGGUUAGCGGCACCC | 137 | UUAUGAGGGUGCCGC | 516 |
| | | | UCAUAA | | UAACCGUG | |
| U136 | 1090 | CDS | GGUUAGCGGCACCCU | 138 | UCUAUGAGGGUGCCG | 517 |
| | | | CAUAGA | | CUAACCGU | |
| U137 | 1097 | CDS | GGCACCCUCAUAGGC | 139 | UUCCAGGCCUAUGAG | 518 |
| | | | CUGGAA | | GGUGCCGC | |
| U138 | 1099 | CDS | CACCCUCAUAGGCCU | 140 | AACUCCAGGCCUAUG | 519 |
| | | | GGAGUU | | AGGGUGCC | |
| U139 | 1100 | CDS | ACCCUCAUAGGCCUG | 141 | AAACUCCAGGCCUAU | 520 |
| | | | GAGUUU | | GAGGGUGC | |
| U140 | 1102 | CDS | CCUCAUAGGCCUGGA | 142 | AUAAACUCCAGGCCU | 521 |
| | | | GUUUAU | | AUGAGGGU | |
| U141 | 1102 | CDS | CCUCAUAGGCCUGGA | 143 | UUAAACUCCAGGCCU | 522 |
| | | | GUUUAA | | AUGAGGGU | |
| U142 | 1106 | CDS | AUAGGCCUGGAGUUU | 144 | UCGAAUAAACUCCAG | 523 |
| | | | AUUCGG | | GCCUAUGA | |
| U143 | 1107 | CDS | UAGGCCUGGAGUUUA | 145 | UCCGAAUAAACUCCA | 524 |
| | | | UUCGGA | | GGCCUAUG | |
| U144 | 1108 | CDS | AGGCCUGGAGUUUAU | 146 | UUCCGAAUAAACUCC | 525 |
| | | | UCGGAA | | AGGCCUAU | |
| U145 | 1111 | CDS | CCUGGAGUUUAUUCG | 147 | UUUUUCCGAAUAAAC | 526 |
| | | | GAAAAA | | UCCAGGCC | |
| U146 | 1112 | CDS | CUGGAGUUUAUUCGG | 148 | UCUUUUCCGAAUAAA | 527 |
| | | | AAAAGA | | CUCCAGGC | |
| U147 | 1113 | CDS | UGGAGUUUAUUCGGA | 149 | UGCUUUUCCGAAUAA | 528 |
| | | | AAAGCC | | ACUCCAGG | |
| U148 | 1118 | CDS | UUUAUUCGGAAAAGC | 150 | UAGCUGGCUUUUCCG | 529 |
| | | | CAGCUG | | AAUAAACU | |
| U149 | 1168 | CDS | GCUGCCCCUGGCGGG | 151 | UACCCACCCGCCAGG | 530 |
| | | | UGGGUA | | GGCAGCAG | |
| U150 | 1169 | CDS | CUGCCCCUGGCGGGU | 152 | UUACCCACCCGCCAG | 531 |
| | | | GGGUAA | | GGGCAGCA | |
| U151 | 1170 | CDS | UGCCCCUGGCGGGUG | 153 | UGUACCCACCCGCCA | 532 |
| | | | GGUACA | | GGGGCAGC | |
| U152 | 1221 | CDS | UGGCGAGGGCUGGGG | 154 | UCACGACCCCAGCCC | 533 |
| | | | UCGUGC | | UCGCCAGG | |
| U153 | 1228 | CDS | GGCUGGGGUCGUGCU | 155 | UUGACCAGCACGACC | 534 |
| | | | GGUCAA | | CCAGCCCU | |
| U154 | 1234 | CDS | GGUCGUGCUGGUCAC | 156 | UCAGCGGUGACCAGC | 535 |
| | | | CGCUGA | | ACGACCCC | |
| U155 | 1237 | CDS | CGUGCUGGUCACCGC | 157 | UCGGCAGCGGUGACC | 536 |
| | | | UGCCGA | | AGCACGAC | |
| U156 | 1238 | CDS | GUGCUGGUCACCGCU | 158 | UCCGGCAGCGGUGAC | 537 |
| | | | GCCGGA | | CAGCACGA | |
| U157 | 1241 | CDS | CUGGUCACCGCUGCC | 159 | UUUGCCGGCAGCGGU | 538 |
| | | | GGCAAA | | GACCAGCA | |
| U158 | 1246 | CDS | CACCGCUGCCGGCAA | 160 | UGGAAGUUGCCGGCA | 539 |
| | | | CUUCCA | | GCGGUGAC | |
| U159 | 1252 | CDS | UGCCGGCAACUUCCG | 161 | UCGUCCCGGAAGUUG | 540 |
| | | | GGACGA | | CCGGCAGC | |
| U160 | 1253 | CDS | GCCGGCAACUUCCGG | 162 | AUCGUCCCGGAAGUU | 541 |
| | | | GACGAU | | GCCGGCAG | |
| U161 | 1253 | CDS | GCCGGCAACUUCCGG | 163 | UUCGUCCCGGAAGUU | 542 |
| | | | GACGAA | | GCCGGCAG | |
| U162 | 1254 | CDS | CCGGCAACUUCCGGG | 164 | UAUCGUCCCGGAAGU | 543 |
| | | | ACGAUA | | UGCCGGCA | |
| U163 | 1255 | CDS | CGGCAACUUCCGGGA | 165 | UCAUCGUCCCGGAAG | 544 |
| | | | CGAUGA | | UUGCCGGC | |
| U164 | 1256 | CDS | GGCAACUUCCGGGAC | 166 | UGCAUCGUCCCGGAA | 545 |
| | | | GAUGCA | | GUUGCCGG | |
| U165 | 1257 | CDS | GCAACUUCCGGGACG | 167 | AGGCAUCGUCCCGGA | 546 |
| | | | AUGCCU | | AGUUGCCG | |
| U166 | 1257 | CDS | GCAACUUCCGGGACG | 168 | UGGCAUCGUCCCGGA | 547 |
| | | | AUGCCA | | AGUUGCCG | |
| U167 | 1258 | CDS | CAACUUCCGGGACGA | 169 | UAGGCAUCGUCCCGG | 548 |
| | | | UGCCUA | | AAGUUGCC | |
| U168 | 1260 | CDS | ACUUCCGGGACGAUG | 170 | UGCAGGCAUCGUCCC | 549 |
| | | | CCUGCC | | GGAAGUUG | |
| U169 | 1292 | CDS | GCCUCAGCUCCCGAG | 171 | UAUGACCUCGGGAGC | 550 |
| | | | GUCAUA | | UGAGGCUG | |
| U170 | 1293 | CDS | CCUCAGCUCCCGAGG | 172 | UGAUGACCUCGGGAG | 551 |
| | | | UCAUCA | | CUGAGGCU | |
| U171 | 1296 | CDS | CAGCUCCCGAGGUCA | 173 | UUGUGAUGACCUCGG | 552 |
| | | | UCACAA | | GAGCUGAG | |
| U172 | 1299 | CDS | CUCCCGAGGUCAUCA | 174 | UAACUGUGAUGACCU | 553 |
| | | | CAGUUA | | CGGGAGCU | |
| U173 | 1308 | CDS | UCAUCACAGUUGGGG | 175 | UGGUGGCCCCAACUG | 554 |
| | | | CCACCA | | UGAUGACC | |
| U174 | 1309 | CDS | CAUCACAGUUGGGGC | 176 | UUGGUGGCCCCAACU | 555 |
| | | | CACCAA | | GUGAUGAC | |
| U175 | 1354 | CDS | GGGGACUUUGGGGAC | 177 | AAGUUGGUCCCCAAA | 556 |
| | | | CAACUU | | GUCCCCAG | |
| U176 | 1354 | CDS | GGGGACUUUGGGGAC | 178 | UAGUUGGUCCCCAAA | 557 |
| | | | CAACUA | | GUCCCCAG | |
| U177 | 1355 | CDS | GGGACUUUGGGGACC | 179 | AAAGUUGGUCCCCAA | 558 |
| | | | AACUUU | | AGUCCCCA | |
| U178 | 1355 | CDS | GGGACUUUGGGGACC | 180 | UAAGUUGGUCCCCAA | 559 |
| | | | AACUUA | | AGUCCCCA | |
| U179 | 1357 | CDS | GACUUUGGGGACCAA | 181 | UCAAAGUUGGUCCCC | 560 |
| | | | CUUUGA | | AAAGUCCC | |
| U180 | 1359 | CDS | CUUUGGGGACCAACU | 182 | UGCCAAAGUUGGUCC | 561 |
| | | | UUGGCA | | CCAAAGUC | |
| U181 | 1361 | CDS | UUGGGGACCAACUUU | 183 | UCGGCCAAAGUUGGU | 562 |
| | | | GGCCGC | | CCCCAAAG | |
| U182 | 1365 | CDS | GGACCAACUUUGGCC | 184 | UACAGCGGCCAAAGU | 563 |
| | | | GCUGUA | | UGGUCCCC | |
| U183 | 1367 | CDS | ACCAACUUUGGCCGC | 185 | UACACAGCGGCCAAA | 564 |
| | | | UGUGUG | | GUUGGUCC | |
| U184 | 1376 | CDS | GGCCGCUGUGUGGAC | 186 | AAAGAGGUCCACACA | 565 |
| | | | CUCUUU | | GCGGCCAA | |
| U185 | 1376 | CDS | GGCCGCUGUGUGGAC | 187 | UAAGAGGUCCACACA | 566 |
| | | | CUCUUA | | GCGGCCAA | |
| U186 | 1378 | CDS | CCGCUGUGUGGACCU | 188 | UCAAAGAGGUCCACA | 567 |
| | | | CUUUGA | | CAGCGGCC | |
| U187 | 1379 | CDS | CGCUGUGUGGACCUC | 189 | UGCAAAGAGGUCCAC | 568 |
| | | | UUUGCA | | ACAGCGGC | |
| U188 | 1401 | CDS | CAGGGGAGGACAUCA | 190 | UACCAAUGAUGUCCU | 569 |
| | | | UUGGUA | | CCCCUGGG | |
| U189 | 1409 | CDS | GACAUCAUUGGUGCC | 191 | UCUGGAGGCACCAAU | 570 |
| | | | UCCAGA | | GAUGUCCU | |
| U190 | 1414 | CDS | CAUUGGUGCCUCCAG | 192 | UAGUCGCUGGAGGCA | 571 |
| | | | CGACUA | | CCAAUGAU | |
| U191 | 1430 | CDS | GACUGCAGCACCUGC | 193 | UACAAAGCAGGUGCU | 572 |
| | | | UUUGUA | | GCAGUCGC | |
| U192 | 1433 | CDS | UGCAGCACCUGCUUU | 194 | UGACACAAAGCAGGU | 573 |
| | | | GUGUCA | | GCUGCAGU | |
| U193 | 1435 | CDS | CAGCACCUGCUUUGU | 195 | UGUGACACAAAGCAG | 574 |
| | | | GUCACA | | GUGCUGCA | |
| U194 | 1478 | CDS | GCCCACGUGGCUGGC | 196 | UGCAAUGCCAGCCAC | 575 |
| | | | AUUGCA | | GUGGGCAG | |
| U195 | 1488 | CDS | CUGGCAUUGCAGCCA | 197 | UCAUCAUGGCUGCAA | 576 |
| | | | UGAUGA | | UGCCAGCC | |
| U196 | 1489 | CDS | UGGCAUUGCAGCCAU | 198 | AGCAUCAUGGCUGCA | 577 |
| | | | GAUGCU | | AUGCCAGC | |
| U197 | 1489 | CDS | UGGCAUUGCAGCCAU | 199 | UGCAUCAUGGCUGCA | 578 |
| | | | GAUGCA | | AUGCCAGC | |
| U198 | 1494 | CDS | UUGCAGCCAUGAUGC | 200 | UAGACAGCAUCAUGG | 579 |
| | | | UGUCUG | | CUGCAAUG | |
| U199 | 1502 | CDS | AUGAUGCUGUCUGCC | 201 | UGGCUCGGCAGACAG | 580 |
| | | | GAGCCG | | CAUCAUGG | |
| U200 | 1504 | CDS | GAUGCUGUCUGCCGA | 202 | UCCGGCUCGGCAGAC | 581 |
| | | | GCCGGA | | AGCAUCAU | |
| U201 | 1505 | CDS | AUGCUGUCUGCCGAG | 203 | UUCCGGCUCGGCAGA | 582 |
| | | | CCGGAG | | CAGCAUCA | |
| U202 | 1522 | CDS | GGAGCUCACCCUGGC | 204 | AACUCGGCCAGGGUG | 583 |
| | | | CGAGUU | | AGCUCCGG | |
| U203 | 1522 | CDS | GGAGCUCACCCUGGC | 205 | UACUCGGCCAGGGUG | 584 |
| | | | CGAGUA | | AGCUCCGG | |
| U204 | 1523 | CDS | GAGCUCACCCUGGCC | 206 | UAACUCGGCCAGGGU | 585 |
| | | | GAGUUA | | GAGCUCCG | |
| U205 | 1526 | CDS | CUCACCCUGGCCGAG | 207 | UCUCAACUCGGCCAG | 586 |
| | | | UUGAGA | | GGUGAGCU | |
| U206 | 1533 | CDS | UGGCCGAGUUGAGGC | 208 | UUCUCUGCCUCAACU | 587 |
| | | | AGAGAC | | CGGCCAGG | |
| U207 | 1538 | CDS | GAGUUGAGGCAGAGA | 209 | UAUCAGUCUCUGCCU | 588 |
| | | | CUGAUA | | CAACUCGG | |
| U208 | 1546 | CDS | GCAGAGACUGAUCCA | 210 | UAGAAGUGGAUCAGU | 589 |
| | | | CUUCUA | | CUCUGCCU | |
| U209 | 1547 | CDS | CAGAGACUGAUCCAC | 211 | AGAGAAGUGGAUCAG | 590 |
| | | | UUCUCU | | UCUCUGCC | |
| U210 | 1547 | CDS | CAGAGACUGAUCCAC | 212 | UGAGAAGUGGAUCAG | 591 |
| | | | UUCUCA | | UCUCUGCC | |
| U211 | 1548 | CDS | AGAGACUGAUCCACU | 213 | UAGAGAAGUGGAUCA | 592 |
| | | | UCUCUG | | GUCUCUGC | |
| U212 | 1562 | CDS | UUCUCUGCCAAAGAU | 214 | UAUGACAUCUUUGGC | 593 |
| | | | GUCAUC | | AGAGAAGU | |
| U213 | 1571 | CDS | AAAGAUGUCAUCAAU | 215 | UGCCUCAUUGAUGAC | 594 |
| | | | GAGGCC | | AUCUUUGG | |
| U214 | 1577 | CDS | GUCAUCAAUGAGGCC | 216 | UAACCAGGCCUCAUU | 595 |
| | | | UGGUUA | | GAUGACAU | |
| U215 | 1578 | CDS | UCAUCAAUGAGGCCU | 217 | UGAACCAGGCCUCAU | 596 |
| | | | GGUUCC | | UGAUGACA | |
| U216 | 1598 | CDS | CCUGAGGACCAGCGG | 218 | UAGUACCCGCUGGUC | 597 |
| | | | GUACUA | | CUCAGGGA | |
| U217 | 1600 | CDS | UGAGGACCAGCGGGU | 219 | UUCAGUACCCGCUGG | 598 |
| | | | ACUGAC | | UCCUCAGG | |
| U218 | 1601 | CDS | GAGGACCAGCGGGUA | 220 | UGUCAGUACCCGCUG | 599 |
| | | | CUGACA | | GUCCUCAG | |
| U219 | 1603 | CDS | GGACCAGCGGGUACU | 221 | UGGGUCAGUACCCGC | 600 |
| | | | GACCCA | | UGGUCCUC | |
| U220 | 1695 | CDS | CAGCACACUCGGGGC | 222 | UUGUAGGCCCCGAGU | 601 |
| | | | CUACAA | | GUGCUGAC | |
| U221 | 1696 | CDS | AGCACACUCGGGGCC | 223 | UGUGUAGGCCCCGAG | 602 |
| | | | UACACG | | UGUGCUGA | |
| U222 | 1697 | CDS | GCACACUCGGGGCCU | 224 | UCGUGUAGGCCCCGA | 603 |
| | | | ACACGA | | GUGUGCUG | |
| U223 | 1698 | CDS | CACACUCGGGGCCUA | 225 | UCCGUGUAGGCCCCG | 604 |
| | | | CACGGA | | AGUGUGCU | |
| U224 | 1699 | CDS | ACACUCGGGGCCUAC | 226 | AUCCGUGUAGGCCCC | 605 |
| | | | ACGGAU | | GAGUGUGC | |
| U225 | 1699 | CDS | ACACUCGGGGCCUAC | 227 | UUCCGUGUAGGCCCC | 606 |
| | | | ACGGAA | | GAGUGUGC | |
| U226 | 1700 | CDS | CACUCGGGGCCUACA | 228 | UAUCCGUGUAGGCCC | 607 |
| | | | CGGAUA | | CGAGUGUG | |
| U227 | 1703 | CDS | UCGGGGCCUACACGG | 229 | UGCCAUCCGUGUAGG | 608 |
| | | | AUGGCC | | CCCCGAGU | |
| U228 | 1708 | CDS | GCCUACACGGAUGGC | 230 | UCUGUGGCCAUCCGU | 609 |
| | | | CACAGA | | GUAGGCCC | |
| U229 | 1709 | CDS | CCUACACGGAUGGCC | 231 | UGCUGUGGCCAUCCG | 610 |
| | | | ACAGCA | | UGUAGGCC | |
| U230 | 1756 | CDS | GCUGCUGAGCUGCUC | 232 | AAACUGGAGCAGCUC | 611 |
| | | | CAGUUU | | AGCAGCUC | |
| U231 | 1756 | CDS | GCUGCUGAGCUGCUC | 233 | UAACUGGAGCAGCUC | 612 |
| | | | CAGUUA | | AGCAGCUC | |
| U232 | 1759 | CDS | GCUGAGCUGCUCCAG | 234 | UAGAAACUGGAGCAG | 613 |
| | | | UUUCUA | | CUCAGCAG | |
| U233 | 1800 | CDS | GCGAGCGCAUGGAGG | 235 | UUUGGGCCUCCAUGC | 614 |
| | | | CCCAAA | | GCUCGCCC | |
| U234 | 1823 | CDS | GGCAAGCUGGUCUGC | 236 | UGCCCGGCAGACCAG | 615 |
| | | | CGGGCA | | CUUGCCCC | |
| U235 | 1829 | CDS | CUGGUCUGCCGGGCC | 237 | UUUGUGGGCCCGGCA | 616 |
| | | | CACAAA | | GACCAGCU | |
| U236 | 1831 | CDS | GGUCUGCCGGGCCCA | 238 | UCGUUGUGGGCCCGG | 617 |
| | | | CAACGA | | CAGACCAG | |
| U237 | 1832 | CDS | GUCUGCCGGGCCCAC | 239 | AGCGUUGUGGGCCCG | 618 |
| | | | AACGCU | | GCAGACCA | |
| U238 | 1832 | CDS | GUCUGCCGGGCCCAC | 240 | UGCGUUGUGGGCCCG | 619 |
| | | | AACGCA | | GCAGACCA | |
| U239 | 1833 | CDS | UCUGCCGGGCCCACA | 241 | AAGCGUUGUGGGCCC | 620 |
| | | | ACGCUU | | GGCAGACC | |
| U240 | 1833 | CDS | UCUGCCGGGCCCACA | 242 | UAGCGUUGUGGGCCC | 621 |
| | | | ACGCUA | | GGCAGACC | |
| U241 | 1835 | CDS | UGCCGGGCCCACAAC | 243 | AAAAGCGUUGUGGGC | 622 |
| | | | GCUUUU | | CCGGCAGA | |
| U242 | 1835 | CDS | UGCCGGGCCCACAAC | 244 | UAAAGCGUUGUGGGC | 623 |
| | | | GCUUUA | | CCGGCAGA | |
| U243 | 1836 | CDS | GCCGGGCCCACAACG | 245 | UAAAAGCGUUGUGGG | 624 |
| | | | CUUUUA | | CCCGGCAG | |
| U244 | 1860 | CDS | GUGAGGGUGUCUACG | 246 | UAAUGGCGUAGACAC | 625 |
| | | | CCAUUA | | CCUCACCC | |
| U245 | 1861 | CDS | UGAGGGUGUCUACGC | 247 | UCAAUGGCGUAGACA | 626 |
| | | | CAUUGC | | CCCUCACC | |
| U246 | 1862 | CDS | GAGGGUGUCUACGCC | 248 | UGCAAUGGCGUAGAC | 627 |
| | | | AUUGCA | | ACCCUCAC | |
| U247 | 1863 | CDS | AGGGUGUCUACGCCA | 249 | UGGCAAUGGCGUAGA | 628 |
| | | | UUGCCA | | CACCCUCA | |
| U248 | 1867 | CDS | UGUCUACGCCAUUGC | 250 | UACCUGGCAAUGGCG | 629 |
| | | | CAGGUG | | UAGACACC | |
| U249 | 1914 | CDS | GCGUCCACACAGCUC | 251 | UUGGUGGAGCUGUGU | 630 |
| | | | CACCAA | | GGACGCUG | |
| U250 | 1938 | CDS | AGGCCAGCAUGGGGA | 252 | UACGGGUCCCCAUGC | 631 |
| | | | CCCGUG | | UGGCCUCA | |
| U251 | 1943 | CDS | AGCAUGGGGACCCGU | 253 | UUGGACACGGGUCCC | 632 |
| | | | GUCCAC | | CAUGCUGG | |
| U252 | 1945 | CDS | CAUGGGGACCCGUGU | 254 | UAGUGGACACGGGUC | 633 |
| | | | CCACUA | | CCCAUGCU | |
| U253 | 1946 | CDS | AUGGGGACCCGUGUC | 255 | UCAGUGGACACGGGU | 634 |
| | | | CACUGC | | CCCCAUGC | |
| U254 | 1977 | CDS | GCCACGUCCUCACAG | 256 | UGCAGCCUGUGAGGA | 635 |
| | | | GCUGCA | | CGUGGCCC | |
| U255 | 2006 | CDS | UGGGAGGUGGAGGAC | 257 | UCCAAGGUCCUCCAC | 636 |
| | | | CUUGGC | | CUCCCAGU | |
| U256 | 2017 | CDS | GGACCUUGGCACCCA | 258 | UGCUUGUGGGUGCCA | 637 |
| | | | CAAGCA | | AGGUCCUC | |
| U257 | 2018 | CDS | GACCUUGGCACCCAC | 259 | UGGCUUGUGGGUGCC | 638 |
| | | | AAGCCA | | AAGGUCCU | |
| U258 | 2020 | CDS | CCUUGGCACCCACAA | 260 | UGCGGCUUGUGGGUG | 639 |
| | | | GCCGCA | | CCAAGGUC | |
| U259 | 2022 | CDS | UUGGCACCCACAAGC | 261 | UAGGCGGCUUGUGGG | 640 |
| | | | CGCCUG | | UGCCAAGG | |
| U260 | 2025 | CDS | GCACCCACAAGCCGC | 262 | UCACAGGCGGCUUGU | 641 |
| | | | CUGUGA | | GGGUGCCA | |
| U261 | 2027 | CDS | ACCCACAAGCCGCCU | 263 | UAGCACAGGCGGCUU | 642 |
| | | | GUGCUG | | GUGGGUGC | |
| U262 | 2038 | CDS | GCCUGUGCUGAGGCC | 264 | UCUCGUGGCCUCAGC | 643 |
| | | | ACGAGA | | ACAGGCGG | |
| U263 | 2039 | CDS | CCUGUGCUGAGGCCA | 265 | ACCUCGUGGCCUCAG | 644 |
| | | | CGAGGU | | CACAGGCG | |
| U264 | 2039 | CDS | CCUGUGCUGAGGCCA | 266 | UCCUCGUGGCCUCAG | 645 |
| | | | CGAGGA | | CACAGGCG | |
| U265 | 2040 | CDS | CUGUGCUGAGGCCAC | 267 | UACCUCGUGGCCUCA | 646 |
| | | | GAGGUA | | GCACAGGC | |
| U266 | 2044 | CDS | GCUGAGGCCACGAGG | 268 | UGCUGACCUCGUGGC | 647 |
| | | | UCAGCA | | CUCAGCAC | |
| U267 | 2045 | CDS | CUGAGGCCACGAGGU | 269 | UGGCUGACCUCGUGG | 648 |
| | | | CAGCCA | | CCUCAGCA | |
| U268 | 2048 | CDS | AGGCCACGAGGUCAG | 270 | UUUGGGCUGACCUCG | 649 |
| | | | CCCAAC | | UGGCCUCA | |
| U269 | 2053 | CDS | ACGAGGUCAGCCCAA | 271 | UACUGGUUGGGCUGA | 650 |
| | | | CCAGUG | | CCUCGUGG | |
| U270 | 2054 | CDS | CGAGGUCAGCCCAAC | 272 | UCACUGGUUGGGCUG | 651 |
| | | | CAGUGA | | ACCUCGUG | |
| U271 | 2086 | CDS | GGAGGCCAGCAUCCA | 273 | UAAGCGUGGAUGCUG | 652 |
| | | | CGCUUA | | GCCUCCCU | |
| U272 | 2120 | CDS | CCAGGUCUGGAAUGC | 274 | UACUUUGCAUUCCAG | 653 |
| | | | AAAGUA | | ACCUGGGG | |
| U273 | 2135 | CDS | AAAGUCAAGGAGCAU | 275 | UAUUCCAUGCUCCUU | 654 |
| | | | GGAAUC | | GACUUUGC | |
| U274 | 2136 | CDS | AAGUCAAGGAGCAUG | 276 | UGAUUCCAUGCUCCU | 655 |
| | | | GAAUCC | | UGACUUUG | |
| U275 | 2140 | CDS | CAAGGAGCAUGGAAU | 277 | UCCGGGAUUCCAUGC | 656 |
| | | | CCCGGA | | UCCUUGAC | |
| U276 | 2248 | CDS | GGCCUACGCCGUAGA | 278 | UUGUUGUCUACGGCG | 657 |
| | | | CAACAA | | UAGGCCCC | |
| U277 | 2249 | CDS | GCCUACGCCGUAGAC | 279 | UGUGUUGUCUACGGC | 658 |
| | | | AACACA | | GUAGGCCC | |
| U278 | 2250 | CDS | CCUACGCCGUAGACA | 280 | ACGUGUUGUCUACGG | 659 |
| | | | ACACGU | | CGUAGGCC | |
| U279 | 2250 | CDS | CCUACGCCGUAGACA | 281 | UCGUGUUGUCUACGG | 660 |
| | | | ACACGA | | CGUAGGCC | |
| U280 | 2251 | CDS | CUACGCCGUAGACAA | 282 | UACGUGUUGUCUACG | 661 |
| | | | CACGUA | | GCGUAGGC | |
| U281 | 2253 | CDS | ACGCCGUAGACAACA | 283 | UACACGUGUUGUCUA | 662 |
| | | | CGUGUG | | CGGCGUAG | |
| U282 | 2254 | CDS | CGCCGUAGACAACAC | 284 | ACACACGUGUUGUCU | 663 |
| | | | GUGUGU | | ACGGCGUA | |
| U283 | 2254 | CDS | CGCCGUAGACAACAC | 285 | UCACACGUGUUGUCU | 664 |
| | | | GUGUGA | | ACGGCGUA | |
| U284 | 2255 | CDS | GCCGUAGACAACACG | 286 | UACACACGUGUUGUC | 665 |
| | | | UGUGUA | | UACGGCGU | |
| U285 | 2274 | CDS | UAGUCAGGAGCCGGG | 287 | UGACGUCCCGGCUCC | 666 |
| | | | ACGUCA | | UGACUACA | |
| U286 | 2277 | CDS | UCAGGAGCCGGGACG | 288 | UGCUGACGUCCCGGC | 667 |
| | | | UCAGCA | | UCCUGACU | |
| U287 | 2293 | CDS | CAGCACUACAGGCAG | 289 | UUGGUGCUGCCUGUA | 668 |
| | | | CACCAA | | GUGCUGAC | |
| U288 | 2296 | CDS | CACUACAGGCAGCAC | 290 | UCGCUGGUGCUGCCU | 669 |
| | | | CAGCGA | | GUAGUGCU | |
| U289 | 2322 | CDS | CCGUGACAGCCGUUG | 291 | AGAUGGCAACGGCUG | 670 |
| | | | CCAUCU | | UCACGGCC | |
| U290 | 2322 | CDS | CCGUGACAGCCGUUG | 292 | UGAUGGCAACGGCUG | 671 |
| | | | CCAUCA | | UCACGGCC | |
| U291 | 2326 | CDS | GACAGCCGUUGCCAU | 293 | UAGCAGAUGGCAACG | 672 |
| | | | CUGCUA | | GCUGUCAC | |
| U292 | 2330 | CDS | GCCGUUGCCAUCUGC | 294 | UCGGCAGCAGAUGGC | 673 |
| | | | UGCCGA | | AACGGCUG | |
| U293 | 2428 | 3′ | GGCUGGGGCUGAGCU | 295 | UUUUAAAGCUCAGCC | 674 |
| | | UTR | UUAAAA | | CCAGCCCU | |
| U294 | 2429 | 3′ | GCUGGGGCUGAGCUU | 296 | AUUUUAAAGCUCAGC | 675 |
| | | UTR | UAAAAU | | CCCAGCCC | |
| U295 | 2429 | 3′ | GCUGGGGCUGAGCUU | 297 | UUUUUAAAGCUCAGC | 676 |
| | | UTR | UAAAAA | | CCCAGCCC | |
| U296 | 2430 | 3′ | CUGGGGCUGAGCUUU | 298 | UAUUUUAAAGCUCAG | 677 |
| | | UTR | AAAAUA | | CCCCAGCC | |
| U297 | 2431 | 3′ | UGGGGCUGAGCUUUA | 299 | UCAUUUUAAAGCUCA | 678 |
| | | UTR | AAAUGG | | GCCCCAGC | |
| U298 | 2432 | 3′ | GGGGCUGAGCUUUAA | 300 | ACCAUUUUAAAGCUC | 679 |
| | | UTR | AAUGGU | | AGCCCCAG | |
| U299 | 2432 | 3′ | GGGGCUGAGCUUUAA | 301 | UCCAUUUUAAAGCUC | 680 |
| | | UTR | AAUGGA | | AGCCCCAG | |
| U300 | 2435 | 3′ | GCUGAGCUUUAAAAU | 302 | UGAACCAUUUUAAAG | 681 |
| | | UTR | GGUUCA | | CUCAGCCC | |
| U301 | 2618 | 3′ | UCCCUCACUGUGGGG | 303 | UAAAUGCCCCACAGU | 682 |
| | | UTR | CAUUUC | | GAGGGAGG | |
| U302 | 2619 | 3′ | CCCUCACUGUGGGGC | 304 | UGAAAUGCCCCACAG | 683 |
| | | UTR | AUUUCA | | UGAGGGAG | |
| U303 | 2621 | 3′ | CUCACUGUGGGGCAU | 305 | UGUGAAAUGCCCCAC | 684 |
| | | UTR | UUCACA | | AGUGAGGG | |
| U304 | 2624 | 3′ | ACUGUGGGGCAUUUC | 306 | AAUGGUGAAAUGCCC | 685 |
| | | UTR | ACCAUU | | CACAGUGA | |
| U305 | 2624 | 3′ | ACUGUGGGGCAUUUC | 307 | UAUGGUGAAAUGCCC | 686 |
| | | UTR | ACCAUA | | CACAGUGA | |
| U306 | 2625 | 3′ | CUGUGGGGCAUUUCA | 308 | UAAUGGUGAAAUGCC | 687 |
| | | UTR | CCAUUA | | CCACAGUG | |
| U307 | 2643 | 3′ | UUCAAACAGGUCGAG | 309 | UCACAGCUCGACCUG | 688 |
| | | UTR | CUGUGC | | UUUGAAUG | |
| U308 | 2763 | 3′ | ACCAAGGAGGCAGGA | 310 | UAAGAAUCCUGCCUC | 689 |
| | | UTR | UUCUUC | | CUUGGUGG | |
| U309 | 2770 | 3′ | AGGCAGGAUUCUUCC | 311 | UCCAUGGGAAGAAUC | 690 |
| | | UTR | CAUGGA | | CUGCCUCC | |
| U310 | 2837 | 3′ | GUGAGUGUGAAAGGU | 312 | AUCAGCACCUUUCAC | 691 |
| | | UTR | GCUGAU | | ACUCACCC | |
| U311 | 2849 | 3′ | GGUGCUGAUGGCCCU | 313 | UAGAUGAGGGCCAUC | 692 |
| | | UTR | CAUCUA | | AGCACCUU | |
| U312 | 2853 | 3′ | CUGAUGGCCCUCAUC | 314 | UCUGGAGAUGAGGGC | 693 |
| | | UTR | UCCAGA | | CAUCAGCA | |
| U313 | 2907 | 3′ | UAAUGGAGGCUUAGC | 315 | UAGAAAGCUAAGCCU | 694 |
| | | UTR | UUUCUG | | CCAUUAAU | |
| U314 | 2916 | 3′ | CUUAGCUUUCUGGAU | 316 | UAUGCCAUCCAGAAA | 695 |
| | | UTR | GGCAUA | | GCUAAGCC | |
| U315 | 2917 | 3′ | UUAGCUUUCUGGAUG | 317 | AGAUGCCAUCCAGAA | 696 |
| | | UTR | GCAUCU | | AGCUAAGC | |
| U316 | 2917 | 3′ | UUAGCUUUCUGGAUG | 318 | UGAUGCCAUCCAGAA | 697 |
| | | UTR | GCAUCA | | AGCUAAGC | |
| U317 | 2918 | 3′ | UAGCUUUCUGGAUGG | 319 | UAGAUGCCAUCCAGA | 698 |
| | | UTR | CAUCUA | | AAGCUAAG | |
| U318 | 2925 | 3′ | CUGGAUGGCAUCUAG | 320 | UUCUGGCUAGAUGCC | 699 |
| | | UTR | CCAGAA | | AUCCAGAA | |
| U319 | 2930 | 3′ | UGGCAUCUAGCCAGA | 321 | UCAGCCUCUGGCUAG | 700 |
| | | UTR | GGCUGG | | AUGCCAUC | |
| U320 | 3035 | 3′ | AACACCCAAAGGUGG | 322 | UGCAGGCCACCUUUG | 701 |
| | | UTR | CCUGCG | | GGUGUUGC | |
| U321 | 3104 | 3′ | CUGUCUCAGCCAACC | 323 | UGAGCGGGUUGGCUG | 702 |
| | | UTR | CGCUCA | | AGACAGUG | |
| U322 | 3105 | 3′ | UGUCUCAGCCAACCC | 324 | UGGAGCGGGUUGGCU | 703 |
| | | UTR | GCUCCA | | GAGACAGU | |
| U323 | 3109 | 3′ | UCAGCCAACCCGCUC | 325 | UUAGUGGAGCGGGUU | 704 |
| | | UTR | CACUAC | | GGCUGAGA | |
| U324 | 3111 | 3′ | AGCCAACCCGCUCCA | 326 | UGGUAGUGGAGCGGG | 705 |
| | | UTR | CUACCC | | UUGGCUGA | |
| U325 | 3190 | 3′ | GCGUGCCUGCCAAGC | 327 | UUGUGAGCUUGGCAG | 706 |
| | | UTR | UCACAA | | GCACGCCC | |
| U326 | 3248 | 3′ | CUGAAGCCAAGCCUC | 328 | UAAGAAGAGGCUUGG | 707 |
| | | UTR | UUCUUA | | CUUCAGAG | |
| U327 | 3251 | 3′ | AAGCCAAGCCUCUUC | 329 | AAGUAAGAAGAGGCU | 708 |
| | | UTR | UUACUU | | UGGCUUCA | |
| U328 | 3251 | 3′ | AAGCCAAGCCUCUUC | 330 | UAGUAAGAAGAGGCU | 709 |
| | | UTR | UUACUA | | UGGCUUCA | |
| U329 | 3252 | 3′ | AGCCAAGCCUCUUCU | 331 | UAAGUAAGAAGAGGC | 710 |
| | | UTR | UACUUC | | UUGGCUUC | |
| U330 | 3314 | 3′ | GGGAAGGGGAACACA | 332 | UUGGUCUGUGUUCCC | 711 |
| | | UTR | GACCAA | | CUUCCCAG | |
| U331 | 3330 | 3′ | ACCAGGAAGCUCGGU | 333 | UCACUCACCGAGCUU | 712 |
| | | UTR | GAGUGA | | CCUGGUCU | |
| U332 | 3333 | 3′ | AGGAAGCUCGGUGAG | 334 | UCAUCACUCACCGAG | 713 |
| | | UTR | UGAUGG | | CUUCCUGG | |
| U333 | 3415 | 3′ | UGGCGGAGAUGCUUC | 335 | UCCUUAGAAGCAUCU | 714 |
| | | UTR | UAAGGC | | CCGCCAGG | |
| U334 | 3455 | 3′ | AACAACUGUCCCUCC | 336 | UCUCAAGGAGGGACA | 715 |
| | | UTR | UUGAGC | | GUUGUUGG | |
| U335 | 3458 | 3′ | AACUGUCCCUCCUUG | 337 | UGUGCUCAAGGAGGG | 716 |
| | | UTR | AGCACC | | ACAGUUGU | |
| U336 | 3475 | 3′ | CACCAGCCCCACCCA | 338 | UUUGCUUGGGUGGGG | 717 |
| | | UTR | AGCAAA | | CUGGUGCU | |
| U337 | 3480 | 3′ | GCCCCACCCAAGCAA | 339 | UUCUGCUUGCUUGGG | 718 |
| | | UTR | GCAGAA | | UGGGGCUG | |
| U338 | 3481 | 3′ | CCCCACCCAAGCAAG | 340 | UGUCUGCUUGCUUGG | 719 |
| | | UTR | CAGACA | | GUGGGGCU | |
| U339 | 3485 | 3′ | ACCCAAGCAAGCAGA | 341 | UAAAUGUCUGCUUGC | 720 |
| | | UTR | CAUUUA | | UUGGGUGG | |
| U340 | 3486 | 3′ | CCCAAGCAAGCAGAC | 342 | AUAAAUGUCUGCUUG | 721 |
| | | UTR | AUUUAU | | CUUGGGUG | |
| U341 | 3486 | 3′ | CCCAAGCAAGCAGAC | 343 | UUAAAUGUCUGCUUG | 722 |
| | | UTR | AUUUAA | | CUUGGGUG | |
| U342 | 3488 | 3′ | CAAGCAAGCAGACAU | 344 | AGAUAAAUGUCUGCU | 723 |
| | | UTR | UUAUCU | | UGCUUGGG | |
| U343 | 3492 | 3′ | CAAGCAGACAUUUAU | 345 | UAAAAGAUAAAUGUC | 724 |
| | | UTR | CUUUUA | | UGCUUGCU | |
| U344 | 3498 | 3′ | GACAUUUAUCUUUUG | 346 | UAGACCCAAAAGAUA | 725 |
| | | UTR | GGUCUA | | AAUGUCUG | |
| U345 | 3501 | 3′ | AUUUAUCUUUUGGGU | 347 | UGACAGACCCAAAAG | 726 |
| | | UTR | CUGUCC | | AUAAAUGU | |
| U346 | 3502 | 3′ | UUUAUCUUUUGGGUC | 348 | AGGACAGACCCAAAA | 727 |
| | | UTR | UGUCCU | | GAUAAAUG | |
| U347 | 3502 | 3′ | UUUAUCUUUUGGGUC | 349 | UGGACAGACCCAAAA | 728 |
| | | UTR | UGUCCA | | GAUAAAUG | |
| U348 | 3505 | 3′ | AUCUUUUGGGUCUGU | 350 | UAGAGGACAGACCCA | 729 |
| | | UTR | CCUCUC | | AAAGAUAA | |
| U349 | 3543 | 3′ | CAACUUUUCUAGACC | 351 | AAAACAGGUCUAGAA | 730 |
| | | UTR | UGUUUU | | AAGUUGGC | |
| U350 | 3543 | 3′ | CAACUUUUCUAGACC | 352 | UAAACAGGUCUAGAA | 731 |
| | | UTR | UGUUUA | | AAGUUGGC | |
| U351 | 3544 | 3′ | AACUUUUCUAGACCU | 353 | UAAAACAGGUCUAGA | 732 |
| | | UTR | GUUUUG | | AAAGUUGG | |
| U352 | 3547 | 3′ | UUUUCUAGACCUGUU | 354 | AAGCAAAACAGGUCU | 733 |
| | | UTR | UUGCUU | | AGAAAAGU | |
| U353 | 3551 | 3′ | CUAGACCUGUUUUGC | 355 | ACAAAAGCAAAACAG | 734 |
| | | UTR | UUUUGU | | GUCUAGAA | |
| U354 | 3551 | 3′ | CUAGACCUGUUUUGC | 356 | UCAAAAGCAAAACAG | 735 |
| | | UTR | UUUUGA | | GUCUAGAA | |
| U355 | 3553 | 3′ | AGACCUGUUUUGCUU | 357 | UUACAAAAGCAAAAC | 736 |
| | | UTR | UUGUAA | | AGGUCUAG | |
| U356 | 3558 | 3′ | UGUUUUGCUUUUGUA | 358 | UCAAGUUACAAAAGC | 737 |
| | | UTR | ACUUGA | | AAAACAGG | |
| U357 | 3559 | 3′ | GUUUUGCUUUUGUAA | 359 | UUCAAGUUACAAAAG | 738 |
| | | UTR | CUUGAA | | CAAAACAG | |
| U358 | 3561 | 3′ | UUUGCUUUUGUAACU | 360 | UCUUCAAGUUACAAA | 739 |
| | | UTR | UGAAGA | | AGCAAAAC | |
| U359 | 3562 | 3′ | UUGCUUUUGUAACUU | 361 | AUCUUCAAGUUACAA | 740 |
| | | UTR | GAAGAU | | AAGCAAAA | |
| U360 | 3562 | 3′ | UUGCUUUUGUAACUU | 362 | UUCUUCAAGUUACAA | 741 |
| | | UTR | GAAGAA | | AAGCAAAA | |
| U361 | 3563 | 3′ | UGCUUUUGUAACUUG | 363 | UAUCUUCAAGUUACA | 742 |
| | | UTR | AAGAUA | | AAAGCAAA | |
| U362 | 3564 | 3′ | GCUUUUGUAACUUGA | 364 | AUAUCUUCAAGUUAC | 743 |
| | | UTR | AGAUAU | | AAAAGCAA | |
| U363 | 3564 | 3′ | GCUUUUGUAACUUGA | 365 | UUAUCUUCAAGUUAC | 744 |
| | | UTR | AGAUAA | | AAAAGCAA | |
| U364 | 3567 | 3′ | UUUGUAACUUGAAGA | 366 | UAAAUAUCUUCAAGU | 745 |
| | | UTR | UAUUUA | | UACAAAAG | |
| U365 | 3568 | 3′ | UUGUAACUUGAAGAU | 367 | AUAAAUAUCUUCAAG | 746 |
| | | UTR | AUUUAU | | UUACAAAA | |
| U366 | 3587 | 3′ | AUUCUGGGUUUUGUA | 368 | AAAUGCUACAAAACC | 747 |
| | | UTR | GCAUUU | | CAGAAUAA | |
| U367 | 3587 | 3′ | AUUCUGGGUUUUGUA | 369 | UAAUGCUACAAAACC | 748 |
| | | UTR | GCAUUA | | CAGAAUAA | |
| U368 | 3590 | 3′ | CUGGGUUUUGUAGCA | 370 | UAAAAAUGCUACAAA | 749 |
| | | UTR | UUUUUA | | ACCCAGAA | |
| U369 | 3595 | 3′ | UUUUGUAGCAUUUUU | 371 | AUUAAUAAAAAUGCU | 750 |
| | | UTR | AUUAAU | | ACAAAACC | |
| U370 | 3597 | 3′ | UUGUAGCAUUUUUAU | 372 | AUAUUAAUAAAAAUG | 751 |
| | | UTR | UAAUAU | | CUACAAAA | |
| U371 | 3600 | 3′ | UAGCAUUUUUAUUAA | 373 | ACCAUAUUAAUAAAA | 752 |
| | | UTR | UAUGGU | | AUGCUACA | |
| U372 | 3604 | 3′ | AUUUUUAUUAAUAUG | 374 | AGUCACCAUAUUAAU | 753 |
| | | UTR | GUGACU | | AAAAAUGC | |
| U373 | 3606 | 3′ | UUUUAUUAAUAUGGU | 375 | AAAGUCACCAUAUUA | 754 |
| | | UTR | GACUUU | | AUAAAAAU | |
| U374 | 3607 | 3′ | UUUAUUAAUAUGGUG | 376 | AAAAGUCACCAUAUU | 755 |
| | | UTR | ACUUUU | | AAUAAAAA | |
| U375 | 3608 | 3′ | UUAUUAAUAUGGUGA | 377 | AAAAAGUCACCAUAU | 756 |
| | | UTR | CUUUUU | | UAAUAAAA | |
| U376 | 3609 | 3′ | UAUUAAUAUGGUGAC | 378 | UAAAAAGUCACCAUA | 757 |
| | | UTR | UUUUUA | | UUAAUAAA | |
| U377 | 3610 | 3′ | AUUAAUAUGGUGACU | 379 | UUAAAAAGUCACCAU | 758 |
| | | UTR | UUUUAA | | AUUAAUAA | |
| U378 | 3612 | 3′ | UAAUAUGGUGACUUU | 380 | UUUUAAAAAGUCACC | 759 |
| | | UTR | UUAAAA | | AUAUUAAU | |
| U379 | 3613 | 3′ | AAUAUGGUGACUUUU | 381 | AUUUUAAAAAGUCAC | 760 |
| | | UTR | UAAAAU | | CAUAUUAA | |

*site of mRNA target is located in human PCSK9 mRNA sequence in SEQ ID NO: 2.

In Table 1, each code (letter, e.g., A, G, C, and U) represents a single ribonucleotide in the dsRNA. In some embodiments, the sequence list may be inclusive of any possible modifications, for example, a modification in a nucleobase, a ribose sugar ring, and/or a phosphate group (i.e., phosphodiester internucleoside linkage). In some embodiments, the last nucleotide from the 5′ end (or the first nucleotide from 3′ end) in each strand (sense strand and antisense strand) may have not include a phosphate group as being hydrolyzed or processed, e.g., during the synthesis of the oligonucleotides, but may contain 3′-terminal —OH group. In some embodiments, a phosphate group in the last nucleotide from the 5′ end (or the first nucleotide from 3′ end) in the sense strand may be added as a functional group for conjugation with a ligand.

In some embodiments, the dsRNA includes a sense strand having 10 contiguous nucleotides differing by no more than 3 nucleotides from the nucleotide sequence selected from SEQ ID NOs: 3 to 381. In some embodiments, the dsRNA includes an antisense strand having 10 contiguous nucleotides differing by no more than 3 nucleotides from the nucleotide sequence of SEQ ID NOs: 382 to 760. In some embodiments, the dsRNA includes a sense strand having 11 contiguous nucleotides differing by no more than 3 nucleotides from the nucleotide sequence selected from SEQ ID NOs: 3 to 381. In some embodiments, the dsRNA includes an antisense strand having 11 contiguous nucleotides differing by no more than 3 nucleotides from the nucleotide sequence selected from SEQ ID NOs: 382 to 760. In some embodiments, the dsRNA includes a sense strand having 12 contiguous nucleotides differing by no more than 3 nucleotides from the nucleotide sequence selected from SEQ ID NOs: 3 to 381. In some embodiments, the dsRNA includes an antisense strand having 12 contiguous nucleotides differing by no more than 3 nucleotides from the nucleotide sequence selected from SEQ ID NOs: 382 to 760. In some embodiments, the dsRNA includes a sense strand having 13 contiguous nucleotides differing by no more than 3 nucleotides from the nucleotide sequence selected from SEQ ID NOs: 3 to 381. In some embodiments, the dsRNA includes an antisense strand having 13 contiguous nucleotides differing by no more than 3 nucleotides from the nucleotide sequence selected from SEQ ID NOs: 382 to 760. In some embodiments, the dsRNA includes a sense strand having 14 contiguous nucleotides differing by no more than 3 nucleotides from the nucleotide sequence selected from SEQ ID NOs: 3 to 381. In some embodiments, the dsRNA includes an antisense strand having 14 contiguous nucleotides differing by no more than 3 nucleotides from the nucleotide sequence selected from SEQ ID NOs: 382 to 760. In some embodiments, the dsRNA includes a sense strand having 15 contiguous nucleotides differing by no more than 3 nucleotides from the nucleotide sequence selected from SEQ ID NOs: 3 to 381. In some embodiments, the dsRNA includes an antisense strand having 15 contiguous nucleotides differing by no more than 3 nucleotides from the nucleotide sequence selected from SEQ ID NOs: 382 to 760. In some embodiments, the dsRNA includes a sense strand having 16 contiguous nucleotides differing by no more than 3 nucleotides from the nucleotide sequence selected from SEQ ID NOs: 3 to 381. In some embodiments, the dsRNA includes an antisense strand having 16 contiguous nucleotides differing by no more than 3 nucleotides from the nucleotide sequence selected from SEQ ID NOs: 382 to 760. In some embodiments, the dsRNA includes a sense strand having 17 contiguous nucleotides differing by no more than 3 nucleotides from the nucleotide sequence selected from SEQ ID NOs: 3 to 381. In some embodiments, the dsRNA includes an antisense strand having 17 contiguous nucleotides differing by no more than 3 nucleotides from the nucleotide sequence selected from SEQ ID NOs: 382 to 760. In some embodiments, the dsRNA includes a sense strand having 18 contiguous nucleotides differing by no more than 3 nucleotides from the nucleotide sequence selected from SEQ ID NOs: 3 to 381. In some embodiments, the dsRNA includes an antisense strand having 18 contiguous nucleotides differing by no more than 3 nucleotides from the nucleotide sequence selected from SEQ ID NOs: 382 to 760. In some embodiments, the dsRNA includes a sense strand having 19 contiguous nucleotides differing by no more than 3 nucleotides from the nucleotide sequence selected from SEQ ID NOs: 3 to 381. In some embodiments, the dsRNA includes an antisense strand having 19 contiguous nucleotides differing by no more than 3 nucleotides from the nucleotide sequence selected from SEQ ID NOs: 382 to 760. In some embodiments, the dsRNA includes a sense strand having 20 contiguous nucleotides differing by no more than 3 nucleotides from the nucleotide sequence selected from SEQ ID NOs: 3 to 381. In some embodiments, the dsRNA includes an antisense strand having 20 contiguous nucleotides differing by no more than 3 nucleotides from the nucleotide sequence selected from SEQ ID NOs: 382 to 760. In some embodiments, the dsRNA includes a sense strand having 21 contiguous nucleotides differing by no more than 3 nucleotides from the nucleotide sequence selected from SEQ ID NOs: 3 to 381. In some embodiments, the dsRNA includes an antisense strand having 21 contiguous nucleotides differing by no more than 3 nucleotides from the nucleotide sequence selected from SEQ ID NOs: 382 to 760.

In some embodiments, the dsRNA includes a sense strand having 10 contiguous nucleotides differing by no more than 2 nucleotides from the nucleotide sequence selected from SEQ ID NOs: 3 to 381. In some embodiments, the dsRNA includes an antisense strand having 10 contiguous nucleotides differing by no more than 2 nucleotides from the nucleotide sequence selected from SEQ ID NOs: 382 to 760. In some embodiments, the dsRNA includes a sense strand having 11 contiguous nucleotides differing by no more than 2 nucleotides from the nucleotide sequence selected from SEQ ID NOs: 3 to 381. In some embodiments, the dsRNA includes an antisense strand having 11 contiguous nucleotides differing by no more than 2 nucleotides from the nucleotide sequence selected from SEQ ID NOs: 382 to 760. In some embodiments, the dsRNA includes a sense strand having 12 contiguous nucleotides differing by no more than 2 nucleotides from the nucleotide sequence selected from SEQ ID NOs: 3 to 381. In some embodiments, the dsRNA includes an antisense strand having 12 contiguous nucleotides differing by no more than 2 nucleotides from the nucleotide sequence selected from SEQ ID NOs: 382 to 760. In some embodiments, the dsRNA includes a sense strand having 13 contiguous nucleotides differing by no more than 2 nucleotides from the nucleotide sequence selected from SEQ ID NOs: 3 to 381. In some embodiments, the dsRNA includes an antisense strand having 13 contiguous nucleotides differing by no more than 2 nucleotides from the nucleotide sequence selected from SEQ ID NOs: 382 to 760. In some embodiments, the dsRNA includes a sense strand having 14 contiguous nucleotides differing by no more than 2 nucleotides from the nucleotide sequence selected from SEQ ID NOs: 3 to 381. In some embodiments, the dsRNA includes an antisense strand having 14 contiguous nucleotides differing by no more than 2 nucleotides from the nucleotide sequence selected from SEQ ID NOs: 382 to 760. In some embodiments, the dsRNA includes a sense strand having 15 contiguous nucleotides differing by no more than 2 nucleotides from the nucleotide sequence selected from SEQ ID NOs: 3 to 381. In some embodiments, the dsRNA includes an antisense strand having 15 contiguous nucleotides differing by no more than 2 nucleotides from the nucleotide sequence selected from SEQ ID NOs: 382 to 760. In some embodiments, the dsRNA includes a sense strand having 16 contiguous nucleotides differing by no more than 2 nucleotides from the nucleotide sequence selected from SEQ ID NOs: 3 to 381. In some embodiments, the dsRNA includes an antisense strand having 16 contiguous nucleotides differing by no more than 2 nucleotides from the nucleotide sequence selected from SEQ ID NOs: 382 to 760. In some embodiments, the dsRNA includes a sense strand having 17 contiguous nucleotides differing by no more than 2 nucleotides from the nucleotide sequence selected from SEQ ID NOs: 3 to 381. In some embodiments, the dsRNA includes an antisense strand having 17 contiguous nucleotides differing by no more than 2 nucleotides from the nucleotide sequence selected from SEQ ID NOs: 382 to 760. In some embodiments, the dsRNA includes a sense strand having 18 contiguous nucleotides differing by no more than 2 nucleotides from the nucleotide sequence selected from SEQ ID NOs: 3 to 381. In some embodiments, the dsRNA includes an antisense strand having 18 contiguous nucleotides differing by no more than 2 nucleotides from the nucleotide sequence selected from SEQ ID NOs: 382 to 760. In some embodiments, the dsRNA includes a sense strand having 19 contiguous nucleotides differing by no more than 2 nucleotides from the nucleotide sequence selected from SEQ ID NOs: 3 to 381. In some embodiments, the dsRNA includes an antisense strand having 19 contiguous nucleotides differing by no more than 2 nucleotides from the nucleotide sequence selected from SEQ ID NOs: 382 to 760. In some embodiments, the dsRNA includes a sense strand having 20 contiguous nucleotides differing by no more than 2 nucleotides from the nucleotide sequence selected from SEQ ID NOs: 3 to 381. In some embodiments, the dsRNA includes an antisense strand having 20 contiguous nucleotides differing by no more than 2 nucleotides from the nucleotide sequence selected from SEQ ID NOs: 382 to 760. In some embodiments, the dsRNA includes a sense strand having 21 contiguous nucleotides differing by no more than 2 nucleotides from the nucleotide sequence selected from SEQ ID NOs: 3 to 381. In some embodiments, the dsRNA includes an antisense strand having 21 contiguous nucleotides differing by no more than 2 nucleotides from the nucleotide sequence selected from SEQ ID NOs: 382 to 760.

In some embodiments, the dsRNA includes a sense strand having 10 contiguous nucleotides differing by no more than 1 nucleotide from the nucleotide sequence selected from SEQ ID NOs: 3 to 381. In some embodiments, the dsRNA includes an antisense strand having 10 contiguous nucleotides differing by no more than 1 nucleotide from the nucleotide sequence selected from SEQ ID NOs: 382 to 760. In some embodiments, the dsRNA includes a sense strand having 11 contiguous nucleotides differing by no more than 1 nucleotide from the nucleotide sequence selected from SEQ ID NOs: 3 to 381. In some embodiments, the dsRNA includes an antisense strand having 11 contiguous nucleotides differing by no more than 1 nucleotide from the nucleotide sequence selected from SEQ ID NOs: 382 to 760. In some embodiments, the dsRNA includes a sense strand having 12 contiguous nucleotides differing by no more than 1 nucleotide from the nucleotide sequence selected from SEQ ID NOs: 3 to 381. In some embodiments, the dsRNA includes an antisense strand having 12 contiguous nucleotides differing by no more than 1 nucleotide from the nucleotide sequence selected from SEQ ID NOs: 382 to 760. In some embodiments, the dsRNA includes a sense strand having 13 contiguous nucleotides differing by no more than 1 nucleotide from the nucleotide sequence selected from SEQ ID NOs: 3 to 381. In some embodiments, the dsRNA includes an antisense strand having 13 contiguous nucleotides differing by no more than 1 nucleotide from the nucleotide sequence selected from SEQ ID NOs: 382 to 760. In some embodiments, the dsRNA includes a sense strand having 14 contiguous nucleotides differing by no more than 1 nucleotide from the nucleotide sequence selected from SEQ ID NOs: 3 to 381. In some embodiments, the dsRNA includes an antisense strand having 14 contiguous nucleotides differing by no more than 1 nucleotide from the nucleotide sequence selected from SEQ ID NOs: 382 to 760. In some embodiments, the dsRNA includes a sense strand having 15 contiguous nucleotides differing by no more than 1 nucleotide from the nucleotide sequence selected from SEQ ID NOs: 3 to 381. In some embodiments, the dsRNA includes an antisense strand having 15 contiguous nucleotides differing by no more than 1 nucleotide from the nucleotide sequence selected from SEQ ID NOs: 382 to 760. In some embodiments, the dsRNA includes a sense strand having 16 contiguous nucleotides differing by no more than 1 nucleotide from the nucleotide sequence selected from SEQ ID NOs: 3 to 381. In some embodiments, the dsRNA includes an antisense strand having 16 contiguous nucleotides differing by no more than 1 nucleotide from the nucleotide sequence selected from SEQ ID NOs: 382 to 760. In some embodiments, the dsRNA includes a sense strand having 17 contiguous nucleotides differing by no more than 1 nucleotide from the nucleotide sequence selected from SEQ ID NOs: 3 to 381. In some embodiments, the dsRNA includes an antisense strand having 17 contiguous nucleotides differing by no more than 1 nucleotide from the nucleotide sequence selected from SEQ ID NOs: 382 to 760. In some embodiments, the dsRNA includes a sense strand having 18 contiguous nucleotides differing by no more than 1 nucleotide from the nucleotide sequence selected from SEQ ID NOs: 3 to 381. In some embodiments, the dsRNA includes an antisense strand having 18 contiguous nucleotides differing by no more than 1 nucleotide from the nucleotide sequence selected from SEQ ID NOs: 382 to 760. In some embodiments, the dsRNA includes a sense strand having 19 contiguous nucleotides differing by no more than 1 nucleotide from the nucleotide sequence selected from SEQ ID NOs: 3 to 381. In some embodiments, the dsRNA includes an antisense strand having 19 contiguous nucleotides differing by no more than 1 nucleotide from the nucleotide sequence selected from SEQ ID NOs: 382 to 760. In some embodiments, the dsRNA includes a sense strand having 20 contiguous nucleotides differing by no more than 1 nucleotide from the nucleotide sequence selected from SEQ ID NOs: 3 to 381. In some embodiments, the dsRNA includes an antisense strand having 20 contiguous nucleotides differing by no more than 1 nucleotide from the nucleotide sequence selected from SEQ ID NOs: 382 to 760. In some embodiments, the dsRNA includes a sense strand having 21 contiguous nucleotides differing by no more than 1 nucleotide from the nucleotide sequence selected from SEQ ID NOs: 3 to 381. In some embodiments, the dsRNA includes an antisense strand having 21 contiguous nucleotides differing by no more than 1 nucleotide from the nucleotide sequence selected from SEQ ID NOs: 382 to 760. In some embodiments, the dsRNA includes an antisense strand having 22 contiguous nucleotides differing by no more than 1 nucleotide from the nucleotide sequence selected from SEQ ID NOs: 382 to 760. In some embodiments, the dsRNA includes an antisense strand having 23 contiguous nucleotides differing by no more than 1 nucleotide from the nucleotide sequence selected from SEQ ID NOs: 382 to 760.

In some embodiments, the dsRNA includes (i) a sense strand having 15 contiguous nucleotides differing by no more than one, two or three nucleotides from the nucleotide sequence selected from SEQ ID NOs: 3 to 381 and (ii) an antisense strand forming a duplex with the sense strand of (i) and having 15 contiguous nucleotides differing by no more than one, two or three nucleotides from the nucleotide sequence selected from SEQ ID NOs: 382 to 760. In some embodiments, the dsRNA includes (i) a sense strand having 16 contiguous nucleotides differing by no more than one, two or three from the nucleotide sequence selected from SEQ ID NOs: 3 to 381 and (ii) an antisense strand forming a duplex with the sense strand of (i) and having 16 contiguous nucleotides differing by no more than one, two or three nucleotides from the nucleotide sequence selected from SEQ ID NOs: 382 to 760. In some embodiments, the dsRNA includes (i) a sense strand having 17 contiguous nucleotides differing by no more than one, two or three nucleotides from the nucleotide sequence selected from SEQ ID NOs: 3 to 381 and (ii) an antisense strand forming a duplex with the sense strand of (i) and having 17 contiguous nucleotides differing by no more than one, two or three nucleotides from the nucleotide sequence selected from SEQ ID NOs: 382 to 760. In some embodiments, the dsRNA includes (i) a sense strand having 18 contiguous nucleotides differing by no more than one, two or three nucleotides from the nucleotide sequence selected from SEQ ID NOs: 3 to 381 and (ii) an antisense strand forming a duplex with the sense strand of (i) and having 18 contiguous nucleotides differing by no more than one, two or three nucleotides from the nucleotide sequence selected from SEQ ID NOs: 382 to 760. In some embodiments, the dsRNA includes (i) a sense strand having 19 contiguous nucleotides differing by no more than one, two or three nucleotides from the nucleotide sequence selected from SEQ ID NOs: 3 to 381 and (ii) an antisense strand forming a duplex with the sense strand of (i) and having 19 contiguous nucleotides differing by no more than one, two or three nucleotides from the nucleotide sequence selected from SEQ ID NOs: 382 to 760. In some embodiments, the dsRNA includes (i) a sense strand having 20 contiguous nucleotides differing by no more than one, two or three nucleotides from the nucleotide sequence selected from SEQ ID NOs: 3 to 381 and (ii) an antisense strand forming a duplex with the sense strand of (i) and having 20 contiguous nucleotides differing by no more than one, two or three nucleotides from the nucleotide sequence selected from SEQ ID NOs: 382 to 760. In some embodiments, the dsRNA includes (i) a sense strand having 21 contiguous nucleotides differing by no more than one, two or three nucleotides from the nucleotide sequence selected from SEQ ID NOs: 3 to 381 and (ii) an antisense strand forming a duplex with the sense strand of (i) and having 21 contiguous nucleotides differing by no more than one, two or three nucleotides from the nucleotide sequence selected from SEQ ID NOs: 382 to 760.

In certain aspects, the sequences of the single strands (i.e., sense strand and antisense strand) of the dsRNA can be selected by selecting a target region and a length in the PCSK9 mRNA. In certain aspects, a dsRNA as described herein may target a nucleotide region selected from regions of (i) 600-800; (ii) 800 to 1000; (iii) 1000-1200; (iv) 3100-3300; or (v) 3400-3600 of a human PCSK9 mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 2 (human PCSK9 isoform, transcript variant 1, mRNA (GenBank: NM_174936.4)). In some embodiments, the target region is selected from regions of (i) 650-750; (ii) 850-950; (iii) 1050-1150; (iv) 3200-3300; (v) 3400-3500; or (vi) 3500-3600 of a human PCSK9 mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 2 (human PCSK9 isoform, transcript variant 1, mRNA (GenBank: NM_174936.4)).

In some embodiments, the antisense strand targets a region of (i) 600-800; (ii) 800-1000; (iii) 1000-1200; (iv) 3100-3300; or (v) 3400-3600 nucleotides in a human PCSK9 mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 2 (human PCSK9 isoform, transcript variant 1, mRNA (GenBank: NM_174936.4)). In some embodiments, the antisense strand targets a region of (i) 650-750; (ii) 850-950; (iii) 1050-1150; (iv) 3200-3300; (v) 3400-3500; or (vi) 3500-3600 nucleotides in a human PCSK9 mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 2 (human PCSK9 isoform, transcript variant 1, mRNA (GenBank: NM_174936.4)). In some embodiments, the antisense strand targets a region of 3500th to 3600th nucleotides in a human PCSK9 mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 2 (human PCSK9 isoform, transcript variant 1, mRNA (GenBank: NM_174936.4)).

Exemplary selected siRNA sequences targeting the regions described herein (e.g., target region of (i) 600-800; (ii) 800 to 1000; (iii) 1000-1200; (iv) 3100-3300; or (v) 3400-3600) are shown in Table 2.

TABLE 2
Selected sequences of PCSK9 siRNA (unmodified nucleotide sequences)

| | Site of | | SEQ | | SEQ |
|---|---|---|---|---|---|
| SiRNA | mRNA | | ID | | ID |
| No. | Target* | Sense strand | NO. | Antisense strand | NO. |
| P1 | 3551 | CUAGACCUGUUUUGCU | 761 | ACAAAAGCAAAACAGGUC | 777 |
| | | UUUGU | | UAGAA | |
| P2 | 3492 | CAAGCAGACAUUUAUC | 762 | UAAAAGAUAAAUGUCUGC | 778 |
| | | UUUUA | | UUGCU | |
| P3 | 3543 | CAACUUUUCUAGACCU | 763 | AAAACAGGUCUAGAAAAG | 779 |
| | | GUUUU | | UUGGC | |
| P4 | 3564 | GCUUUUGUAACUUGAA | 764 | AUAUCUUCAAGUUACAAA | 780 |
| | | GAUAU | | AGCAA | |
| P5 | 713 | UUGAAGUUGCCCCAUG | 765 | UUCGACAUGGGGCAACUU | 781 |
| | | UCGAA | | CAAGG | |
| P6 | 1102 | CCUCAUAGGCCUGGAG | 766 | AUAAACUCCAGGCCUAUG | 782 |
| | | UUUAU | | AGGGU | |
| P7 | 3251 | AAGCCAAGCCUCUUCU | 767 | AAGUAAGAAGAGGCUUGG | 783 |
| | | UACUU | | CUUCA | |
| P8 | 3252 | AGCCAAGCCUCUUCUU | 768 | UAAGUAAGAAGAGGCUUG | 784 |
| | | ACUUA | | GCUUC | |
| P9 | 3547 | UUUUCUAGACCUGUUU | 769 | AAGCAAAACAGGUCUAGA | 785 |
| | | UGCUU | | AAAGU | |
| P10 | 3553 | AGACCUGUUUUGCUUU | 770 | UUACAAAAGCAAAACAGG | 786 |
| | | UGUAA | | UCUAG | |
| P11 | 3568 | UUGUAACUUGAAGAUA | 771 | AUAAAUAUCUUCAAGUUA | 787 |
| | | UUUAU | | CAAAA | |
| P12 | 3559 | GUUUUGCUUUUGUAAC | 772 | UUCAAGUUACAAAAGCAA | 788 |
| | | UUGAA | | | |
| P13 | 432 | UGGUGCUAGCCUUGCG | 773 | UGGAACGCAAGGCUAGCA | 789 |
| | | UUCCA | | CCAGC | |
| P14 | 881 | AGUGACCACCGGGAAA | 774 | UUCGAUUUCCCGGUGGUC | 790 |
| | | UCGAA | | ACUCU | |
| P15 | 2907 | UAAUGGAGGCUUAGCU | 775 | UAGAAAGCUAAGCCUCCA | 791 |
| | | UUCUA | | UUAAU | |
| P16 | 865 | AGACACCAGCAUACAG | 776 | UCACUCUGUAUGCUGGUG | 792 |
| | | AGUGA | | UCUAG | |

*site of mRNA target is located in human PCSK9 mRNA sequence in SEQ ID NO: 2.

In some embodiments, the dsRNA includes a sense strand having 10 contiguous nucleotides differing by no more than 3 nucleotides from the nucleotide sequence selected from SEQ ID NOs: 761 to 776. In some embodiments, the dsRNA includes an antisense strand having 10 contiguous nucleotides differing by no more than 3 nucleotides from the nucleotide sequence of SEQ ID NOs: 777 to 792. In some embodiments, the dsRNA includes a sense strand having 11 contiguous nucleotides differing by no more than 3 nucleotides from the nucleotide sequence selected from SEQ ID NOs: 761 to 776. In some embodiments, the dsRNA includes an antisense strand having 11 contiguous nucleotides differing by no more than 3 nucleotides from the nucleotide sequence selected from SEQ ID NOs: 777 to 792. In some embodiments, the dsRNA includes a sense strand having 12 contiguous nucleotides differing by no more than 3 nucleotides from the nucleotide sequence selected from SEQ ID NOs: 761 to 776. In some embodiments, the dsRNA includes an antisense strand having 12 contiguous nucleotides differing by no more than 3 nucleotides from the nucleotide sequence selected from SEQ ID NOs: 777 to 792. In some embodiments, the dsRNA includes a sense strand having 13 contiguous nucleotides differing by no more than 3 nucleotides from the nucleotide sequence selected from SEQ ID NOs: 761 to 776. In some embodiments, the dsRNA includes an antisense strand having 13 contiguous nucleotides differing by no more than 3 nucleotides from the nucleotide sequence selected from SEQ ID NOs: 777 to 792. In some embodiments, the dsRNA includes a sense strand having 14 contiguous nucleotides differing by no more than 3 nucleotides from the nucleotide sequence selected from SEQ ID NOs: 761 to 776. In some embodiments, the dsRNA includes an antisense strand having 14 contiguous nucleotides differing by no more than 3 nucleotides from the nucleotide sequence selected from SEQ ID NOs: 777 to 792. In some embodiments, the dsRNA includes a sense strand having 15 contiguous nucleotides differing by no more than 3 nucleotides from the nucleotide sequence selected from SEQ ID NOs: 761 to 776. In some embodiments, the dsRNA includes an antisense strand having 15 contiguous nucleotides differing by no more than 3 nucleotides from the nucleotide sequence selected from SEQ ID NOs: 777 to 792. In some embodiments, the dsRNA includes a sense strand having 16 contiguous nucleotides differing by no more than 3 nucleotides from the nucleotide sequence selected from SEQ ID NOs: 761 to 776. In some embodiments, the dsRNA includes an antisense strand having 16 contiguous nucleotides differing by no more than 3 nucleotides from the nucleotide sequence selected from SEQ ID NOs: 777 to 792. In some embodiments, the dsRNA includes a sense strand having 17 contiguous nucleotides differing by no more than 3 nucleotides from the nucleotide sequence selected from SEQ ID NOs: 761 to 776. In some embodiments, the dsRNA includes an antisense strand having 17 contiguous nucleotides differing by no more than 3 nucleotides from the nucleotide sequence selected from SEQ ID NOs: 777 to 792. In some embodiments, the dsRNA includes a sense strand having 18 contiguous nucleotides differing by no more than 3 nucleotides from the nucleotide sequence selected from SEQ ID NOs: 761 to 776. In some embodiments, the dsRNA includes an antisense strand having 18 contiguous nucleotides differing by no more than 3 nucleotides from the nucleotide sequence selected from SEQ ID NOs: 777 to 792. In some embodiments, the dsRNA includes a sense strand having 19 contiguous nucleotides differing by no more than 3 nucleotides from the nucleotide sequence selected from SEQ ID NOs: 761 to 776. In some embodiments, the dsRNA includes an antisense strand having 19 contiguous nucleotides differing by no more than 3 nucleotides from the nucleotide sequence selected from SEQ ID NOs: 777 to 792. In some embodiments, the dsRNA includes a sense strand having 20 contiguous nucleotides differing by no more than 3 nucleotides from the nucleotide sequence selected from SEQ ID NOs: 761 to 776. In some embodiments, the dsRNA includes an antisense strand having 20 contiguous nucleotides differing by no more than 3 nucleotides from the nucleotide sequence selected from SEQ ID NOs: 777 to 792. In some embodiments, the dsRNA includes a sense strand having 21 contiguous nucleotides differing by no more than 3 nucleotides from the nucleotide sequence selected from SEQ ID NOs: 761 to 776. In some embodiments, the dsRNA includes an antisense strand having 21 contiguous nucleotides differing by no more than 3 nucleotides from the nucleotide sequence selected from SEQ ID NOs: 777 to 792.

In some embodiments, the dsRNA includes a sense strand having 10 contiguous nucleotides differing by no more than 2 nucleotides from the nucleotide sequence selected from SEQ ID NOs: 761 to 776. In some embodiments, the dsRNA includes an antisense strand having 10 contiguous nucleotides differing by no more than 2 nucleotides from the nucleotide sequence selected from SEQ ID NOs: 777 to 792. In some embodiments, the dsRNA includes a sense strand having 11 contiguous nucleotides differing by no more than 2 nucleotides from the nucleotide sequence selected from SEQ ID NOs: 761 to 776. In some embodiments, the dsRNA includes an antisense strand having 11 contiguous nucleotides differing by no more than 2 nucleotides from the nucleotide sequence selected from SEQ ID NOs: 777 to 792. In some embodiments, the dsRNA includes a sense strand having 12 contiguous nucleotides differing by no more than 2 nucleotides from the nucleotide sequence selected from SEQ ID NOs: 761 to 776. In some embodiments, the dsRNA includes an antisense strand having 12 contiguous nucleotides differing by no more than 2 nucleotides from the nucleotide sequence selected from SEQ ID NOs: 777 to 792. In some embodiments, the dsRNA includes a sense strand having 13 contiguous nucleotides differing by no more than 2 nucleotides from the nucleotide sequence selected from SEQ ID NOs: 761 to 776. In some embodiments, the dsRNA includes an antisense strand having 13 contiguous nucleotides differing by no more than 2 nucleotides from the nucleotide sequence selected from SEQ ID NOs: 777 to 792. In some embodiments, the dsRNA includes a sense strand having 14 contiguous nucleotides differing by no more than 2 nucleotides from the nucleotide sequence selected from SEQ ID NOs: 761 to 776. In some embodiments, the dsRNA includes an antisense strand having 14 contiguous nucleotides differing by no more than 2 nucleotides from the nucleotide sequence selected from SEQ ID NOs: 777 to 792. In some embodiments, the dsRNA includes a sense strand having 15 contiguous nucleotides differing by no more than 2 nucleotides from the nucleotide sequence selected from SEQ ID NOs: 761 to 776. In some embodiments, the dsRNA includes an antisense strand having 15 contiguous nucleotides differing by no more than 2 nucleotides from the nucleotide sequence selected from SEQ ID NOs: 777 to 792. In some embodiments, the dsRNA includes a sense strand having 16 contiguous nucleotides differing by no more than 2 nucleotides from the nucleotide sequence selected from SEQ ID NOs: 761 to 776. In some embodiments, the dsRNA includes an antisense strand having 16 contiguous nucleotides differing by no more than 2 nucleotides from the nucleotide sequence selected from SEQ ID NOs: 777 to 792. In some embodiments, the dsRNA includes a sense strand having 17 contiguous nucleotides differing by no more than 2 nucleotides from the nucleotide sequence selected from SEQ ID NOs: 761 to 776. In some embodiments, the dsRNA includes an antisense strand having 17 contiguous nucleotides differing by no more than 2 nucleotides from the nucleotide sequence selected from SEQ ID NOs: 777 to 792. In some embodiments, the dsRNA includes a sense strand having 18 contiguous nucleotides differing by no more than 2 nucleotides from the nucleotide sequence selected from SEQ ID NOs: 761 to 776. In some embodiments, the dsRNA includes an antisense strand having 18 contiguous nucleotides differing by no more than 2 nucleotides from the nucleotide sequence selected from SEQ ID NOs: 777 to 792. In some embodiments, the dsRNA includes a sense strand having 19 contiguous nucleotides differing by no more than 2 nucleotides from the nucleotide sequence selected from SEQ ID NOs: 761 to 776. In some embodiments, the dsRNA includes an antisense strand having 19 contiguous nucleotides differing by no more than 2 nucleotides from the nucleotide sequence selected from SEQ ID NOs: 777 to 792. In some embodiments, the dsRNA includes a sense strand having 20 contiguous nucleotides differing by no more than 2 nucleotides from the nucleotide sequence selected from SEQ ID NOs: 761 to 776. In some embodiments, the dsRNA includes an antisense strand having 20 contiguous nucleotides differing by no more than 2 nucleotides from the nucleotide sequence selected from SEQ ID NOs: 777 to 792. In some embodiments, the dsRNA includes a sense strand having 21 contiguous nucleotides differing by no more than 2 nucleotides from the nucleotide sequence selected from SEQ ID NOs: 761 to 776. In some embodiments, the dsRNA includes an antisense strand having 21 contiguous nucleotides differing by no more than 2 nucleotides from the nucleotide sequence selected from SEQ ID NOs: 777 to 792.

In some embodiments, the dsRNA includes a sense strand having 10 contiguous nucleotides differing by no more than 1 nucleotide from the nucleotide sequence selected from SEQ ID NOs: 761 to 776. In some embodiments, the dsRNA includes an antisense strand having 10 contiguous nucleotides differing by no more than 1 nucleotide from the nucleotide sequence selected from SEQ ID NOs: 777 to 792. In some embodiments, the dsRNA includes a sense strand having 11 contiguous nucleotides differing by no more than 1 nucleotide from the nucleotide sequence selected from SEQ ID NOs: 761 to 776. In some embodiments, the dsRNA includes an antisense strand having 11 contiguous nucleotides differing by no more than 1 nucleotide from the nucleotide sequence selected from SEQ ID NOs: 777 to 792. In some embodiments, the dsRNA includes a sense strand having 12 contiguous nucleotides differing by no more than 1 nucleotide from the nucleotide sequence selected from SEQ ID NOs: 761 to 776. In some embodiments, the dsRNA includes an antisense strand having 12 contiguous nucleotides differing by no more than 1 nucleotide from the nucleotide sequence selected from SEQ ID NOs: 777 to 792. In some embodiments, the dsRNA includes a sense strand having 13 contiguous nucleotides differing by no more than 1 nucleotide from the nucleotide sequence selected from SEQ ID NOs: 761 to 776. In some embodiments, the dsRNA includes an antisense strand having 13 contiguous nucleotides differing by no more than 1 nucleotide from the nucleotide sequence selected from SEQ ID NOs: 777 to 792. In some embodiments, the dsRNA includes a sense strand having 14 contiguous nucleotides differing by no more than 1 nucleotide from the nucleotide sequence selected from SEQ ID NOs: 761 to 776. In some embodiments, the dsRNA includes an antisense strand having 14 contiguous nucleotides differing by no more than 1 nucleotide from the nucleotide sequence selected from SEQ ID NOs: 777 to 792. In some embodiments, the dsRNA includes a sense strand having 15 contiguous nucleotides differing by no more than 1 nucleotide from the nucleotide sequence selected from SEQ ID NOs: 761 to 776. In some embodiments, the dsRNA includes an antisense strand having 15 contiguous nucleotides differing by no more than 1 nucleotide from the nucleotide sequence selected from SEQ ID NOs: 777 to 792. In some embodiments, the dsRNA includes a sense strand having 16 contiguous nucleotides differing by no more than 1 nucleotide from the nucleotide sequence selected from SEQ ID NOs: 761 to 776. In some embodiments, the dsRNA includes an antisense strand having 16 contiguous nucleotides differing by no more than 1 nucleotide from the nucleotide sequence selected from SEQ ID NOs: 777 to 792. In some embodiments, the dsRNA includes a sense strand having 17 contiguous nucleotides differing by no more than 1 nucleotide from the nucleotide sequence selected from SEQ ID NOs: 761 to 776. In some embodiments, the dsRNA includes an antisense strand having 17 contiguous nucleotides differing by no more than 1 nucleotide from the nucleotide sequence selected from SEQ ID NOs: 777 to 792. In some embodiments, the dsRNA includes a sense strand having 18 contiguous nucleotides differing by no more than 1 nucleotide from the nucleotide sequence selected from SEQ ID NOs: 761 to 776. In some embodiments, the dsRNA includes an antisense strand having 18 contiguous nucleotides differing by no more than 1 nucleotide from the nucleotide sequence selected from SEQ ID NOs: 777 to 792. In some embodiments, the dsRNA includes a sense strand having 19 contiguous nucleotides differing by no more than 1 nucleotide from the nucleotide sequence selected from SEQ ID NOs: 761 to 776. In some embodiments, the dsRNA includes an antisense strand having 19 contiguous nucleotides differing by no more than 1 nucleotide from the nucleotide sequence selected from SEQ ID NOs: 777 to 792. In some embodiments, the dsRNA includes a sense strand having 20 contiguous nucleotides differing by no more than 1 nucleotide from the nucleotide sequence selected from SEQ ID NOs: 761 to 776. In some embodiments, the dsRNA includes an antisense strand having 20 contiguous nucleotides differing by no more than 1 nucleotide from the nucleotide sequence selected from SEQ ID NOs: 777 to 792. In some embodiments, the dsRNA includes a sense strand having 21 contiguous nucleotides differing by no more than 1 nucleotide from the nucleotide sequence selected from SEQ ID NOs: 761 to 776. In some embodiments, the dsRNA includes an antisense strand having 21 contiguous nucleotides differing by no more than 1 nucleotide from the nucleotide sequence selected from SEQ ID NOs: 777 to 792. In some embodiments, the dsRNA includes an antisense strand having 22 contiguous nucleotides differing by no more than 1 nucleotide from the nucleotide sequence selected from SEQ ID NOs: 777 to 792. In some embodiments, the dsRNA includes an antisense strand having 23 contiguous nucleotides differing by no more than 1 nucleotide from the nucleotide sequence selected from SEQ ID NOs: 777 to 792.

In some embodiments, the dsRNA includes (i) a sense strand having 15 contiguous nucleotides differing by no more than one, two or three nucleotides from the nucleotide sequence selected from SEQ ID NOs: 761 to 776 and (ii) an antisense strand forming a duplex with the sense strand of (i) and having 15 contiguous nucleotides differing by no more than one, two or three nucleotides from the nucleotide sequence selected from SEQ ID NOs: 777 to 792. In some embodiments, the dsRNA includes (i) a sense strand having 16 contiguous nucleotides differing by no more than one, two or three from the nucleotide sequence selected from SEQ ID NOs: 761 to 776 and (ii) an antisense strand forming a duplex with the sense strand of (i) and having 16 contiguous nucleotides differing by no more than one, two or three nucleotides from the nucleotide sequence selected from SEQ ID NOs: 777 to 792. In some embodiments, the dsRNA includes (i) a sense strand having 17 contiguous nucleotides differing by no more than one, two or three nucleotides from the nucleotide sequence selected from SEQ ID NOs: 761 to 776 and (ii) an antisense strand forming a duplex with the sense strand of (i) and having 17 contiguous nucleotides differing by no more than one, two or three nucleotides from the nucleotide sequence selected from SEQ ID NOs: 777 to 792. In some embodiments, the dsRNA includes (i) a sense strand having 18 contiguous nucleotides differing by no more than one, two or three nucleotides from the nucleotide sequence selected from SEQ ID NOs: 761 to 776 and (ii) an antisense strand forming a duplex with the sense strand of (i) and having 18 contiguous nucleotides differing by no more than one, two or three nucleotides from the nucleotide sequence selected from SEQ ID NOs: 777 to 792. In some embodiments, the dsRNA includes (i) a sense strand having 19 contiguous nucleotides differing by no more than one, two or three nucleotides from the nucleotide sequence selected from SEQ ID NOs: 761 to 776 and (ii) an antisense strand forming a duplex with the sense strand of (i) and having 19 contiguous nucleotides differing by no more than one, two or three nucleotides from the nucleotide sequence selected from SEQ ID NOs: 777 to 792. In some embodiments, the dsRNA includes (i) a sense strand having 20 contiguous nucleotides differing by no more than one, two or three nucleotides from the nucleotide sequence selected from SEQ ID NOs: 761 to 776 and (ii) an antisense strand forming a duplex with the sense strand of (i) and having 20 contiguous nucleotides differing by no more than one, two or three nucleotides from the nucleotide sequence selected from SEQ ID NOs: 777 to 792. In some embodiments, the dsRNA includes (i) a sense strand having 21 contiguous nucleotides differing by no more than one, two or three nucleotides from the nucleotide sequence selected from SEQ ID NOs: 761 to 776 and (ii) an antisense strand forming a duplex with the sense strand of (i) and having 21 contiguous nucleotides differing by no more than one, two or three nucleotides from the nucleotide sequence selected from SEQ ID NOs: 777 to 792.

In certain aspects, when a sense strand or an antisense strand of a dsRNA in above paragraphs is differing by a certain number of nucleotides (e.g., one, two or three nucleotides) from a specific sequence (e.g., SEQ ID NOs: 3 to 792), it is meant by that the sense strand or the antisense strand of the dsRNA includes one, two or three nucleotides having different nucleobases compared to the nucleobases of the nucleotides at the corresponding positions of the specific sequence (e.g., SEQ ID NOs: 3 to 792).

### Modification Pattern

In an aspect, the disclosure provides a set of modification patterns determined or arranged by modified nucleotides in dsRNAs described herein. Aside from or in addition to the nucleobase sequences, various arrangements of modified nucleotides and the modification patterns thereof can be introduced, for example, to increase stability in a biological or physiological surrounding, to facilitate or promote cleavage by the RNA-induced silencing complex, and/or to mitigate or reduce off-targeting risk (e.g., to PCSK9 off-targeting risk).

In an aspect, the disclosure provides a dsRNA that is partially (e.g., greater than about 1%, 5%, 10%, 15%, 20%, 25%, 30%, 35%, 40%, or 45% of the total nucleotides), substantially (e.g., greater than about 50%, 55%, 60%, 65%, 70%, 75%, 80%, 85%, 90%, or 95% of the total nucleotides), or entirely made of modified nucleotides, which can provide improved resistance to chemical and/or nuclease digestion and increased in vivo stability thereby imposing a longer in vivo half-life. Further, increasing the in vivo half-life of the dsRNA results in enhanced bioavailability and enhanced effectiveness in inhibiting expression or activity of a target gene (e.g., human PCSK9). For example, the stability of dsRNA in blood or serum may be determined, e.g., by its susceptibility to degradation by the cellular enzymes, which may be dependent on the characteristics (e.g., sequences, modification, modification pattern, or other chemical moieties) of each strand (i.e., sense strand or antisense strand) of the dsRNA. In certain aspects, the efficiency of dsRNA as a therapeutic agent may be improved by increasing the in vivo stability (e.g., in blood or serum) of the dsRNA while maintaining the ability of the dsRNA to mediate RNA interference in vivo.

### Modified Nucleotides

The modified nucleotides as used herein contain one or more modifications, for example, the modified nucleotides contain at least one chemical modification or replacement in an internucleoside linkage (“linkage”), a nucleobase, and/or a sugar moiety of the nucleotide. Non-limiting examples include a 2′-modification on a ribose sugar ring (e.g., 2′-deoxy, 2′-O-alkyl, 2′-halo, 2′-O-alkoxyalkyl, 2′-O-amino alkyl, etc.), 3′-modification (e.g., substitution) in backbone phosphate group (or phosphodiester linkage), or 4′-modification on a ribose sugar ring (e.g., 4′-thio RNA). Also, other non-limiting examples of modifications may include one or more modifications selected from a deoxy modification, a 2′-O-alkyl modification, a 2′-halo modification, a 2′-5′-linkage modification, a conformationally restricting modification, an abasic modification, a 2′-amino-modification, a 2′-O-allyl modification, 2′-C-alkyl modification, a 2′-O-alkoxyalkyl modification, a morpholino modification, a modification containing a phosphoramidate group, a non-natural nucleobase modification, a modification in a tetrahydropyran, a threofuranosyl nucleotide (TNA) modification, a modification containing a 1,5-anhydrohexitol, a modification containing a cyclohexyl, a modification containing a cyclohexenyl, a modification containing a phosphorothioate group, a modification containing a methylphosphonate group, a modification containing an alkylphosphate, a modification containing a phosphonate, a modification containing an alkylphosphonate, a modification to form a thermally destabilizing nucleotide, a glycol nucleic acid (GNA) modification, and a 2-O—(N-methylacetamide) modification. For example, a modified nucleotide may include a single modification, or two or more modifications at the positions at which the chemical modification groups do not hinder or intervene each other.

In some embodiments, each of the modified nucleotides is independently selected from TNA, GNA, LNA, 2′-O-alkoxyalkyl modified nucleotide, 2′-O-alkyl modified nucleotide, 2′-O-allyl modified nucleotide, 2′-C-allyl modified nucleotide, 2′-halo modified nucleotide, and 2′-deoxy modified nucleotide (DNA). In some embodiments, each of the modified nucleotides contain independently selected from TNA modification, GNA modification, LNA modification, 2′-O-alkoxyalkyl modification, 2′-O-alkyl modification, 2′-O-allyl modification, 2′-C-allyl modification, 2′-halo modification, and 2′-deoxy modification (DNA). The term alkyl, alkoxyl, allyl, amino, and halo can be interpreted as described above. In some embodiments, the modified nucleotides include at least one TNAs. In some embodiments, the modified nucleotides include at least one GNAs. In some embodiments, the modified nucleotides include at least one LNAs. In some embodiments, the modified nucleotides include at least one 2′-O-alkoxyalkyl modified nucleotides. In some embodiments, the modified nucleotides include at least one 2′-O-alkyl modified nucleotides. In some embodiments, the modified nucleotides include at least one 2′-O-allyl modified nucleotides. In some embodiments, the modified nucleotides include at least one 2′-C-allyl modified nucleotides. In some embodiments, the modified nucleotides include at least one 2′-halo (e.g., —F) modified nucleotides. In some embodiments, the modified nucleotides include at least one 2′-deoxy modified nucleotides (DNA).

In some embodiments, the modified nucleotide may be a bicyclic (or bridged) nucleic acid (“BNA”) having a covalent linkage between the 2′ and 4′ carbons on a ribose sugar. In some embodiments, the modified nucleotide is a locked RNA (“LNA”) having covalent linkage of a bicyclic sugar modification is a 4′-CH₂—O-2′ linkage (methylene oxy), also known as “LNA having a structure of e.g.,

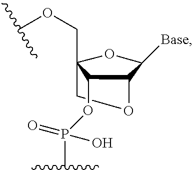

or a pharmaceutically acceptable salt thereof, wherein [symbol] is an attachment point to the adjacent nucleotides.

In some embodiments, a ribose ring may be replaced with a glycol moiety linked to phosphate and the GNA includes a moiety of

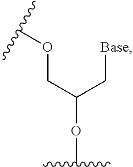

or a pharmaceutically acceptable salt thereof. In some embodiments, the GNA may have a structure of

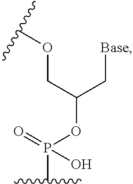

or a pharmaceutically acceptable salt thereof. In some embodiments, the phosphodiester linkage in the GNA may be modified, e.g., with phosphorothioate group and modified GNA may have the structure of

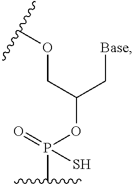

or a pharmaceutically acceptable salt thereof. The GNA may further include one or more substituents replacing hydrogen(s) and such modified GNA may be encompassed by the definition of GNA herein.

In some embodiments, a ribose pentofuranosyl ring may be replaced with a threofuranosyl ring linked to the phosphate and a threofuranosyl nucleotide (TNA) may include a moiety of

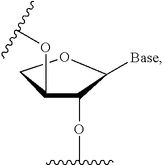

or a pharmaceutically acceptable salt thereof. In some embodiments, the TNA may have a structure of

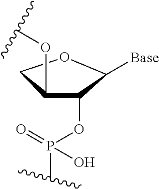

or a pharmaceutically acceptable salt thereof. In some embodiments, the phosphodiester linkage in the TNA may be modified, e.g., with phosphorothioate group and modified TNA may include a structure of

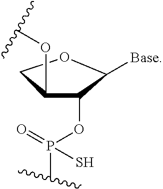

or a pharmaceutically acceptable salt thereof. The TNA may further include one or more substituents at 1′, 3′ and/or 4′ positions and such modified TNA may be encompassed by the definition of TNA herein.

In certain aspects, the modified nucleotide may include a heterocyclic group (e.g., 5 to 6 membered heterocycloalkyl ring) in place of a ribose ring. In some embodiments, the ribose ring may be replaced with a morpholinyl ring, e.g., to form an morpholino oligonucleotide. In some embodiments, the ribose ring may be replaced with an arabinose ring.

In certain aspects, the modified nucleotides contain one or more modification groups at 2′ position on the ribose ring by replacing 2′-OH. In some embodiments, the modification group may include one or more selected from hydrogen (i.e. deoxy), halogen (e.g., —F), substituted or unsubstituted alkyl (e.g., C₁-C₁₂ alkyl), substituted or unsubstituted heteroalkyl (e.g., —O—(C₁-C₁₂ alkyl), —N—(C₁-C₁₂ alkyl), —C(O)NH—(C₁-C₁₂ alkyl), —NHC(O)—(C₁-C₁₂ alkyl), and —C(O)—(C₁-C₁₂ alkyl)). In some embodiments, the modification group may be hydrogen, —F, —O-alkyl (e.g., C₁-C₄ alkyl), or —O-alkoxyalkyl (e.g., —O—(C₁-C₄ alkylene)-(C₁-C₄ alkoxyl)). Any of the alkyl, heteroalkyl, alkylene in the disclosure are optionally substituted with one or more of hydroxyl (—OH), C₁-C₃ alkyl (e.g., methyl, or ethyl), amine (e.g., monoamine or diamine), alkoxyl (e.g., —O—CH₃ (OMe) or —O—CH₂CH₃ (OEt)), halogen (e.g., —F) or the like.

In certain aspects, the modified nucleotides may include one or more of 2′-deoxy modification, 2′-O-alkyl modification, 2′-O-substituted alkyl modification, 2′-O-alkoxyalkyl modification, and 2′-O-aminoalkyl modification. In some embodiments, the modified nucleotides may include one or more of 2′-deoxy modification, 2′-O-alkyl modification, 2′-O-substituted alkyl modification, 2′-O-alkoxyalkyl modification, and 2′-O-aminoalkyl modification. In some embodiments, the modified nucleotides include at least one GNAs. In some embodiments, the modified nucleotides include at least one 2′-O-alkoxyalkyl modifications. In some embodiments, the modified nucleotides include at least one 2′-O-alkyl modifications. In some embodiments, the modified nucleotides include at least one 2′-O-allyl modifications. In some embodiments, the modified nucleotides include at least one 2′-C-allyl modifications. In some embodiments, the modified nucleotides include at least one 2′-halo (e.g., —F) modifications. In some embodiments, the modified nucleotides include at least one 2′-deoxy modifications (DNA). In some embodiments, the modified nucleotides do not include 2′-deoxy modifications (DNA).

In certain aspects, the modified nucleotides may include one or more of 2′-deoxy nucleotide (DNA), 2′-O-methyl (2′-OMe) modification, 2′-flouro (2′-F) modification, 2′-O-methoxyethyl (2′-O-MOE or “2′-MOE”) modification, 2′-O-aminopropyl (2′-O-AP) modification, 2′-O-dimethylaminoethyl (2′-O-DMAOE) modification, 2′-O-dimethylaminopropyl (2′-O-DMAP) modification, 2′-O-dimethylaminoethyloxyethyl (2′-O-DMAEOE) modification, and 2′-O—N-methylacetamido (2′-O-NMA) modification. In some embodiments, the modified nucleotides may include at least one 2′-deoxy modification (DNA). In some embodiments, the modified nucleotides may include at least one 2′-O-methyl (2′-OMe) modification. In some embodiments, the modified nucleotides may include at least one 2′-flouro (2′-F) modification. In some embodiments, the modified nucleotides may include at least one 2′-O-methoxyethyl (2′-O-MOE or “2′-MOE”) modification. In some embodiments, the modified nucleotides may include at least one 2′-O-aminopropyl (2′-O-AP) modification. In some embodiments, the modified nucleotides may include at least one 2′-O-dimethylaminoethyl (2′-O-DMAOE) modification. In some embodiments, the modified nucleotides may include at least one 2′-O-dimethylaminopropyl (2′-O-DMAP) modification. In some embodiments, the modified nucleotides may include at least one 2′-O-dimethylaminoethyloxyethyl (2′-O-DMAEOE) modification. In some embodiments, the modified nucleotides may include at least one 2′-O—N-methylacetamido (2′-O-NMA) modification.

In some embodiments, each modified nucleotide containing a modification on a 2′ sugar ring may optionally contain a phosphorothioate group at 5′ or 3′ linkage. In some embodiments, each modified nucleotide containing a modification on a 2′ sugar ring may optionally contain a modification such as an abasic modification (absence of a nucleobase) or methylated nucleobase modification at nucleobase (e.g., thymine (T) or 5-methyl cytosine (5mC)).

In certain aspects, the dsRNA is partially (e.g., greater than about 1%, 5%, 10%, 15%, 20%, 25%, 30%, 35%, 40%, or 45% of the total nucleotides), substantially (e.g., greater than about 50%, 55%, 60%, 65%, 70%, 75%, 80%, 85%, 90%, or 95% of the total nucleotides), or entirely made of modified nucleotides containing the modification on 2′ sugar ring. In some embodiments, the dsRNA is partially (e.g., greater than about 1%, 5%, 10%, 15%, 20%, 25%, 30%, 35%, 40%, or 45% of the total nucleotides) made of modified nucleotides containing the modification on 2′ sugar ring. In some embodiments, the dsRNA is substantially (e.g., greater than about 50%, 55%, 60%, 65%, 70%, 75%, 80%, 85%, 90%, or 95% of the total nucleotides) made of modified nucleotides containing the modification on 2′ sugar ring. In some embodiments, the dsRNA includes greater than about 80% of modified nucleotides containing the modification on 2′ sugar ring based on the total nucleotides. In some embodiments, the dsRNA includes greater than about 85% of modified nucleotides containing the modification on 2′ sugar ring based on the total nucleotides. In some embodiments, the dsRNA includes greater than about 90% of modified nucleotides containing the modification on 2′ sugar ring based on the total nucleotides. In some embodiments, the dsRNA includes greater than about 95% of modified nucleotides containing the modification on 2′ sugar ring based on the total nucleotides. In some embodiments, the dsRNA is entirely made of modified nucleotides containing the modification on 2′ sugar ring.

In certain aspects, the modified nucleotide may include a modification in a phosphate or phosphodiester linkage, in other words, an internucleoside linkage modification (e.g., phosphorothioate, phosphorodithioate, methylphosphonate, methylene phosphonate, or vinyl phosphonate (VP) linkage). In some embodiments, the linkage modification may include phosphorothioate (PS) having a structure of

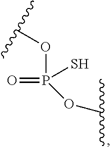 [SMILES: *OP(=O)(S)O*]

which may be an Rp isomer or an Sp isomer. In some embodiments, the linkage modification may include phosphorothioate (PS) having a structure of

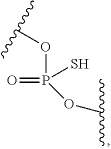 [SMILES: *OP(=O)(S)O*]

which may be a stereopure Rp isomer. In some embodiments, the linkage modification may include phosphorothioate (PS) having a structure of

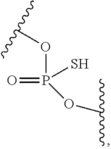 [SMILES: *OP(=O)(S)O*]

which may be a stereopure Sp isomer.

For example, the modified nucleotide including 3′-PS modification can be represented as

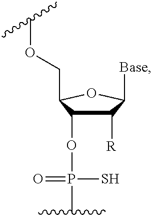

or a pharmaceutically acceptable salt thereof, wherein R represents H, OH or a substituent (e.g., —F, —CH₃, —OMe, or MOE) and [symbol] is an attachment point to the adjacent nucleotides. In some embodiments, the 3′-PS group may be a stereopure Sp isomer. In some embodiments, the 3′-PS group may be a stereopure Rp isomer.

In certain aspects, the dsRNAi agent may be entirely made of modified nucleotides having one or more internucleoside linkage modification and/or modifications in the sugar moieties of the nucleotides.

In certain aspects, the first nucleotide from the 5′ end of each strand (e.g., sense strand and antisense strand) may include an additional phosphate group or a variant thereof (e.g., phosphorothioate, phosphorodithioate, methylphosphonate, methylene phosphonate, or vinyl phosphonate (VP)) attached or linked to the 5′ terminal group of the first nucleotide.

In some embodiments, the first nucleotide from the 5′ end of each strand (e.g., sense strand and antisense strand) includes a 5′-vinyl phosphonate (5′-VP) group that is a chemical moiety having the structure of

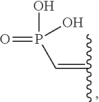 [SMILES: *=CP(=O)(O)O]

or a pharmaceutically acceptable salt thereof, wherein [symbol] represents the point of attachment to the 5′ carbon of the pentofuranosyl sugar of a nucleotide. In some embodiments, the first nucleotide from the 5′ end of each strand (e.g., sense strand and antisense strand) may include (E)-vinyl phosphonate (VP) having a structure of

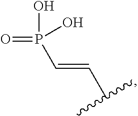 [SMILES: */C=C/P(=O)(O)O]

or a pharmaceutically acceptable salt thereof, wherein [symbol] represents the point of attachment to the 4′ carbon of the pentofuranosyl sugar. In some embodiments, the first nucleotide from the 5′ end of each strand (e.g., sense strand and antisense strand) may include (Z)-vinyl phosphonate having a structure of

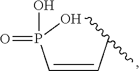 [SMILES: */C=C\P(=O)(O)O]

or a pharmaceutically acceptable salt thereof, wherein [symbol] represents the point of attachment to the 4′ carbon of the pentofuranosyl sugar.

In certain aspects, one or more of the modified nucleotides contain a 2′ modification (e.g., 2′-OMe, 2′-F, 2′-MOE, 2′-deoxy, etc.) and an internucleoside linkage modification (e.g., phosphorothioate or (E)-vinyl phosphonate). In some embodiments, one or more of the modified nucleotides contain 2′-OMe modification and phosphorothioate group. In some embodiments, one or more of the modified nucleotides contain 2′-OMe modification and (E)-vinyl phosphonate group. In some embodiments, one or more of the modified nucleotides contain 2′-F modification and phosphorothioate group. In some embodiments, one or more of the modified nucleotides contain 2′-F and (E)-vinyl phosphonate group. In some embodiments, one or more of the modified nucleotides contain 2′-MOE modification and phosphorothioate group. In some embodiments, one or more of the modified nucleotides contain 2′-MOE modification and (E)-vinyl phosphonate group. In some embodiments, one or more of the modified nucleotides contain 2′-deoxy modification and phosphorothioate group. In some embodiments, one or more of the modified nucleotides contain 2′-OMe modification and (E)-vinyl phosphonate group. In some embodiments, one or more of the modified nucleotides are GNA containing (E)-vinyl phosphonate group. In some embodiments, one or more of the modified nucleotides are GNA containing a phosphorothioate group. In some embodiments, one or more of the modified nucleotides are TNA containing (E)-vinyl phosphonate group. In some embodiments, one or more of the modified nucleotides are TNA containing a phosphorothioate group.

In certain aspects, the modified nucleotides contain one or more modifications on a modified nucleobase. In some embodiments, one or more of the modified nucleotides may include thymine (“T”) nucleobase (“ribothymidine” or “5-methyluridine”) in the ribonucleotide (e.g., including 2′-OH). In some embodiments, one or more of the modified nucleotides may include methylcytosine nucleobase (e.g., 5-methylcytidine or N4-methylcytidine). In certain aspects, one or more of the modified nucleotides may contain no nucleobase or be abasic.

### Sense Strand (SS)

In certain aspects, a sense strand of the dsRNA as described herein are substantially (e.g., greater than about 80%, 85%, 90%, or 95% of the total nucleotides) made of modified nucleotides. In another certain aspect, the sense strand is entirely made of modified nucleotides.

In certain aspects, a sense strand of the dsRNA as described herein includes two or more 2′-MOE modifications. In some embodiments, the sense strand includes two, four, six or eight 2′-MOE modifications. In some embodiments, the sense strand includes two 2′-MOE modifications. In some embodiments, the sense strand includes four 2′-MOE modifications. In some embodiments, the sense strand includes six 2′-MOE modifications. In some embodiments, the sense strand includes eight 2′-MOE modifications.

In some embodiments, the 2′-MOE modified nucleotides in the sense strand as described herein include a structure of

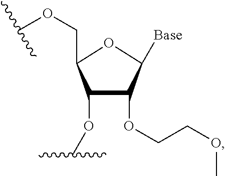

or a pharmaceutically acceptable salt thereof, wherein [symbol] is an attachment point to a linkage (e.g., phosphate or phosphorothioate group) or the adjacent nucleotides and “Base” is a nucleobase.

In some embodiments, the 2′-MOE modified nucleotides in the sense strand as described herein include a structure of

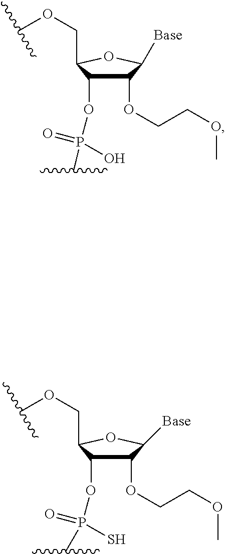

or a pharmaceutically acceptable salt thereof, wherein [symbol] is an attachment point to a terminal group (e.g., H, OH, or salt) or the adjacent nucleotides and “Base” is a nucleobase. In some embodiments, the 2′-MOE modified nucleotides in the sense strand as described herein include a structure of

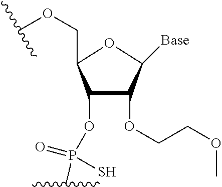

or a pharmaceutically acceptable salt thereof.

In some embodiments, the 2′-MOE modified nucleotides include a structure of

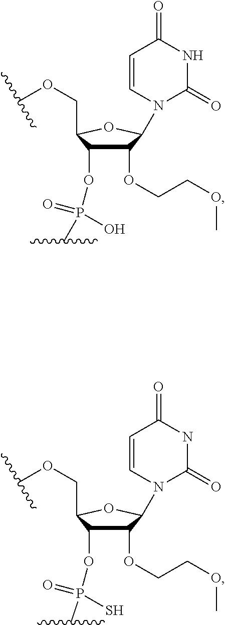

or a pharmaceutically acceptable salt thereof, wherein [symbol] is an attachment point to a terminal group (e.g., H, OH, or salt) or the adjacent nucleotides. In some embodiments, the 2′-MOE modified nucleotides include a structure of

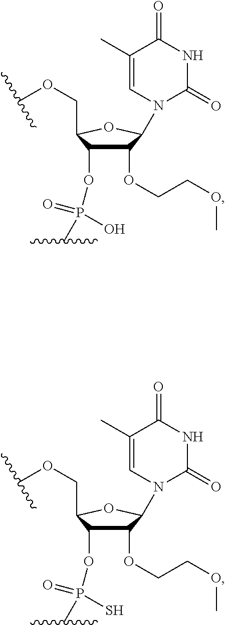

or a pharmaceutically acceptable salt thereof. In some embodiments, the 2′-MOE modified nucleotides include a structure of

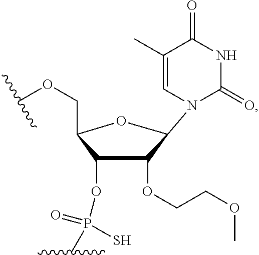

or a pharmaceutically acceptable salt thereof.

In some embodiments, the 2′-MOE modified nucleotides include a nucleotide having a structure of

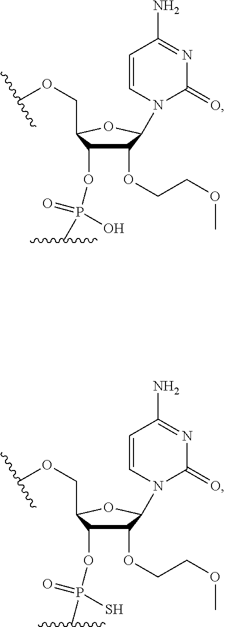

or a pharmaceutically acceptable salt thereof, wherein [symbol] is an attachment point to a terminal group (e.g., H, OH, or salt) or the adjacent nucleotides. In some embodiments, the 2′-MOE modified nucleotides include a nucleotide having a structure of

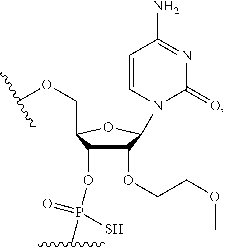

or a pharmaceutically acceptable salt thereof. In some embodiments, the 2′-MOE modified nucleotides include a nucleotide having a structure of

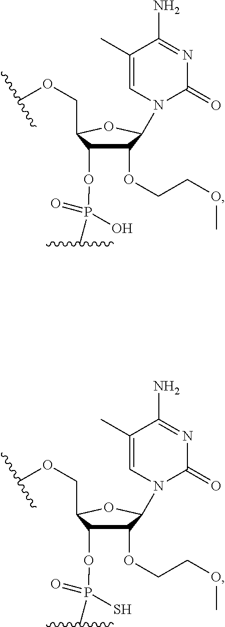

or a pharmaceutically acceptable salt thereof. In some embodiments, the 2′-MOE modified nucleotides include a nucleotide having a structure of

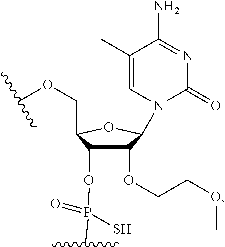

or a pharmaceutically acceptable salt thereof.

In some embodiments, the 2′-MOE modified nucleotides in the sense strand as described herein include a nucleotide having a structure of

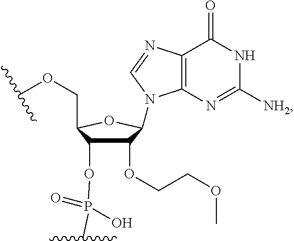

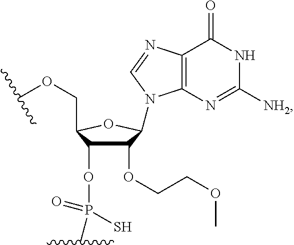

or a pharmaceutically acceptable salt thereof, wherein [symbol] is an attachment point to a terminal group (e.g., H, OH, or salt) or the adjacent nucleotides. In some embodiments, the 2′-MOE modified nucleotides in the sense strand as described herein include a nucleotide having a structure of

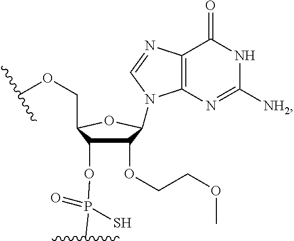

or a pharmaceutically acceptable salt thereof.

In some embodiments, the 2′-MOE modified nucleotides include a nucleotide having a structure of

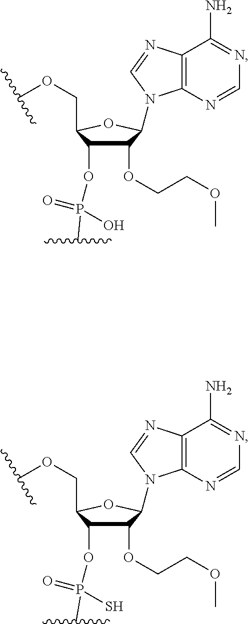

or a pharmaceutically acceptable salt thereof, wherein [symbol] is an attachment point to a terminal group (e.g., H, OH, or salt) or the adjacent nucleotides. In some embodiments, the 2′-MOE modified nucleotides include a nucleotide having a structure of

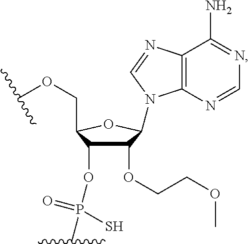

or a pharmaceutically acceptable salt thereof.

In certain aspects, at least one of the 2′-MOE modified nucleotides in the sense strand as described herein has a structure of

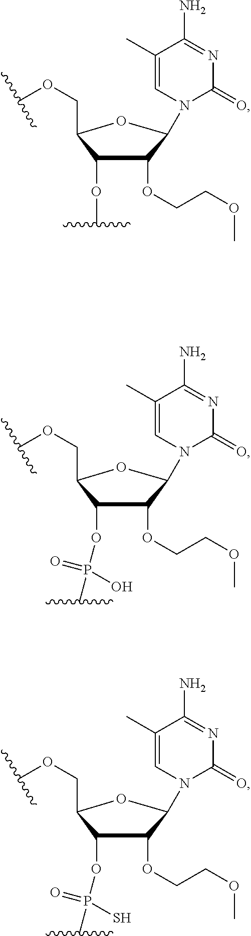

or a pharmaceutically acceptable salt thereof, wherein [symbol] is an attachment point to a terminal group (e.g., H, OH, or salt) or the adjacent nucleotides. In some embodiments, at least one of the 2′-MOE modified nucleotides in the sense strand as described herein has a structure of

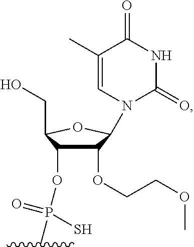

or a pharmaceutically acceptable salt thereof.

In some embodiments, the first nucleotide from the 5′ end of the sense strand includes a structure of

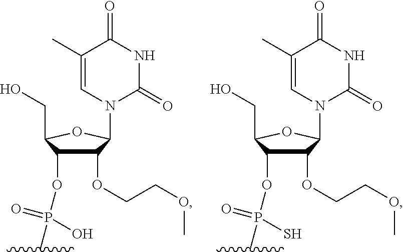

or a pharmaceutically acceptable salt thereof. In some embodiments, the first nucleotide from the 5′ end of the sense strand includes a structure of

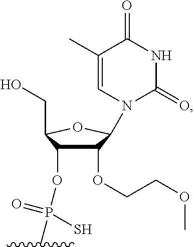

or a pharmaceutically acceptable salt thereof.

In some embodiments, the first nucleotide from the 3′ end of the sense strand includes a structure of

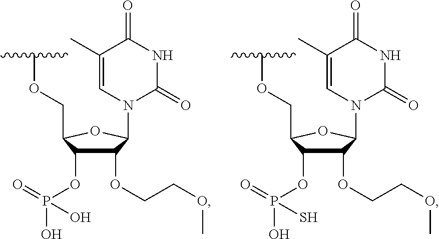

or a pharmaceutically acceptable salt thereof. In some embodiments, the first nucleotide from the 3′ end of the sense strand includes a structure of

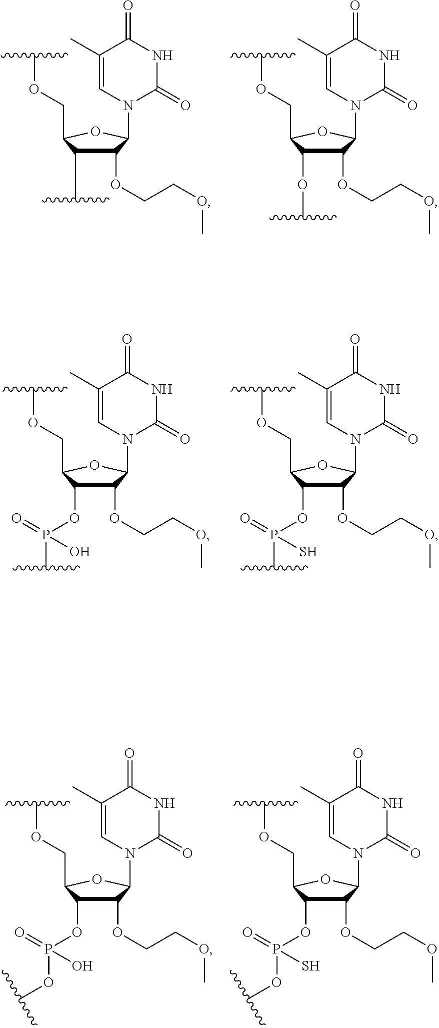

or a pharmaceutically acceptable salt thereof, wherein [symbol] is an attachment point to a ligand. In some embodiments, the first nucleotide from the 3′ end of the sense strand includes a structure of

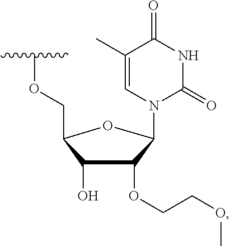

or a pharmaceutically acceptable salt thereof.

In certain aspects, at least one of the 2′-MOE modified nucleotides in the sense strand as described herein has a structure of

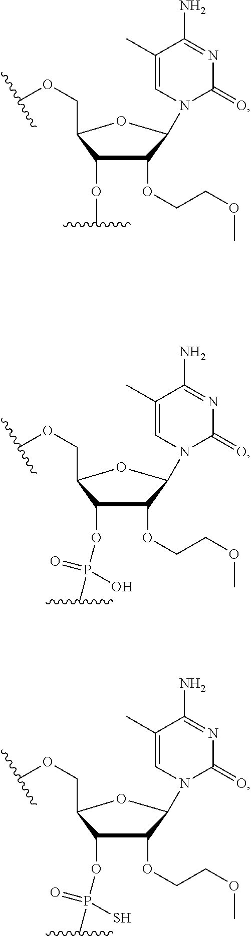

or a pharmaceutically acceptable salt thereof, wherein [symbol] is an attachment point to a terminal group (e.g., H, OH, or salt) or the adjacent nucleotides. In some embodiments, at least one of the 2′-MOE modified nucleotides in the sense strand as described herein has a structure of

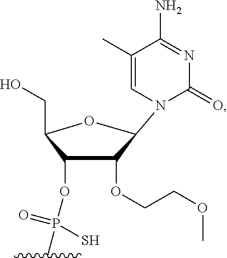

or a pharmaceutically acceptable salt thereof.

In some embodiments, the first nucleotide from the 5′ end of the sense strand includes a structure of

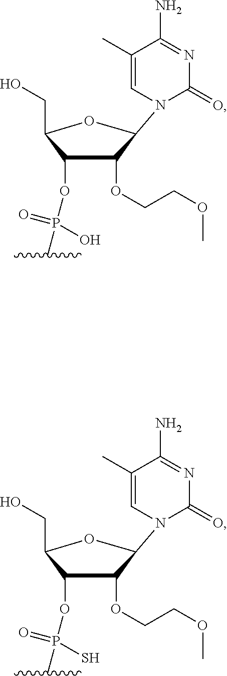

or a pharmaceutically acceptable salt thereof. In some embodiments, the first nucleotide from the 5′ end of the sense strand includes a structure of

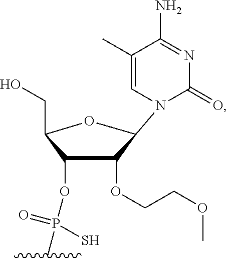

or a pharmaceutically acceptable salt thereof.

In some embodiments, the first nucleotide from the 3′ end of the sense strand includes a structure of

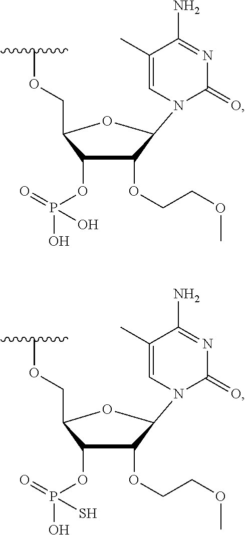

or a pharmaceutically acceptable salt thereof. In some embodiments, the first nucleotide from the 3′ end of the sense strand includes a structure of

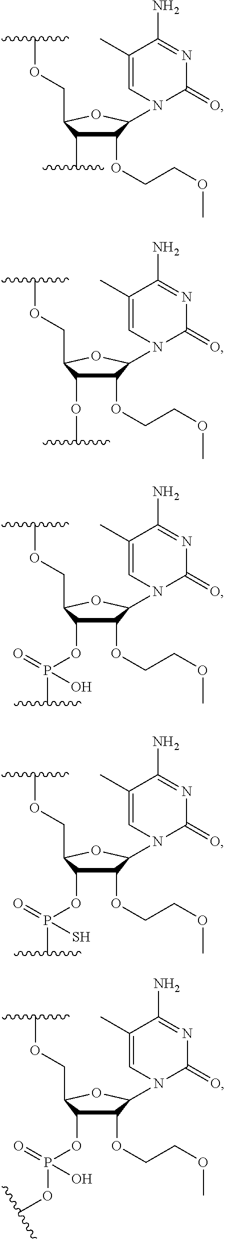

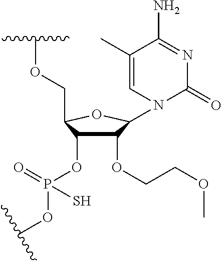

or a pharmaceutically acceptable salt thereof, wherein [symbol] is an attachment point to a ligand. In some embodiments, the first nucleotide from the 3′ end of the sense strand includes a structure of

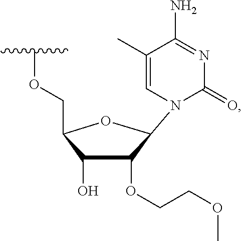

or a pharmaceutically acceptable salt thereof.

In certain aspects, at least one of the 2′-MOE modified nucleotides in the sense strand as described herein has a structure of

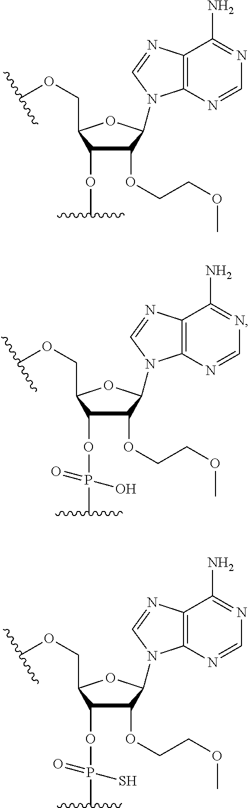

or a pharmaceutically acceptable salt thereof, wherein [symbol] is an attachment point to a terminal group (e.g., H, OH, or salt) or the adjacent nucleotides. In some embodiments, at least one of the 2′-MOE modified nucleotides in the sense strand as described herein has a structure of

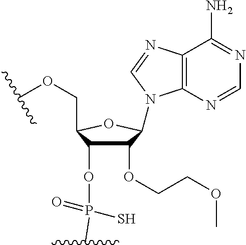

or a pharmaceutically acceptable salt thereof.

In some embodiments, the first nucleotide from the 5′ end of the sense strand includes a structure of

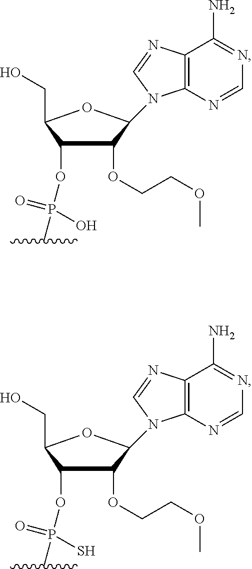

or a pharmaceutically acceptable salt thereof. In some embodiments, the first nucleotide from the 5′ end of the sense strand includes a structure of

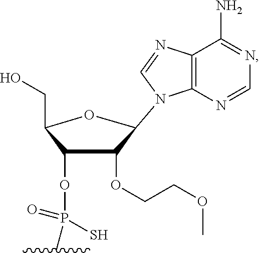

or a pharmaceutically acceptable salt thereof.

In some embodiments, the first nucleotide from the 3′ end of the sense strand has a structure of

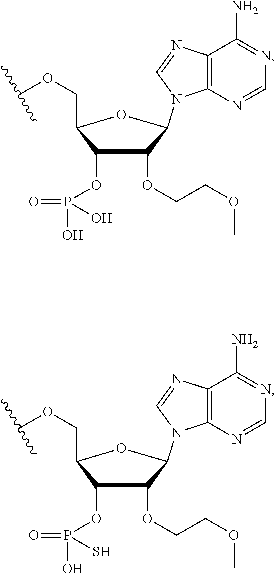

or a pharmaceutically acceptable salt thereof. In some embodiments, the first nucleotide from the 3′ end of the sense strand has a structure of

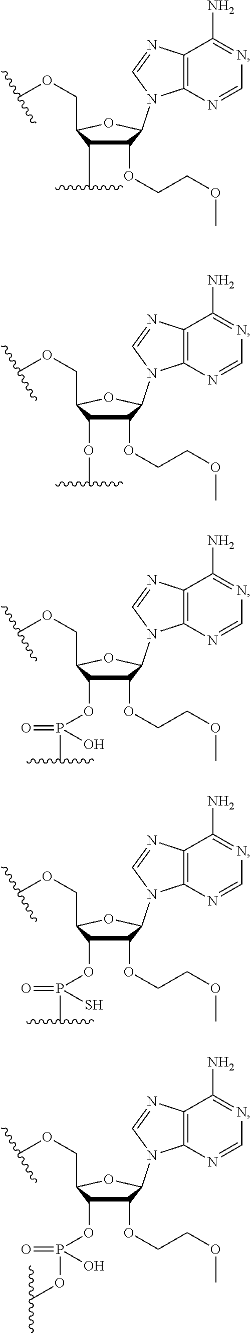

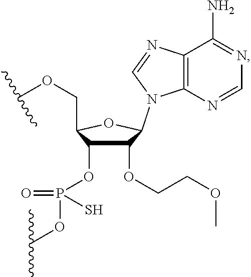

or a pharmaceutically acceptable salt thereof, wherein [symbol] is an attachment point to a ligand. In some embodiments, the first nucleotide from the 3′ end of the sense strand has a structure of

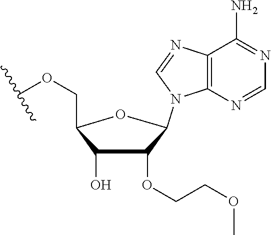

or a pharmaceutically acceptable salt thereof.

In certain aspects, at least one of the 2′-MOE modified nucleotides in the sense strand as described herein has a structure of

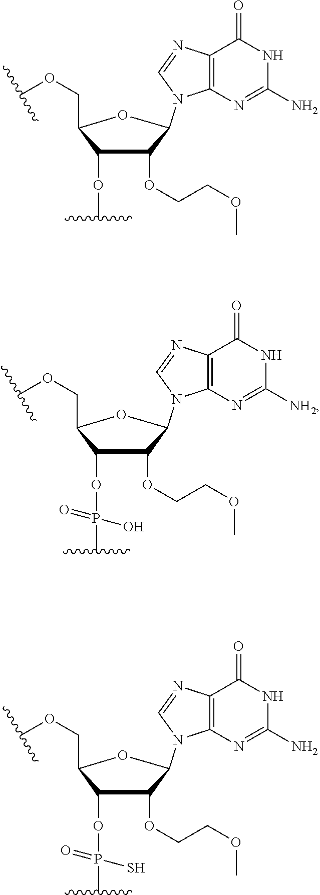

or a pharmaceutically acceptable salt thereof, wherein [symbol] is an attachment point to a terminal group (e.g., H, OH, or salt) or the adjacent nucleotides. In some embodiments, at least one of the 2′-MOE modified nucleotides in the sense strand as described herein has a structure of

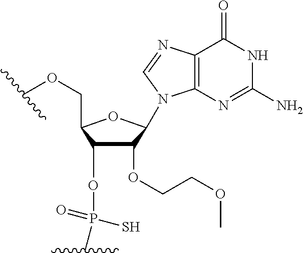

for a pharmaceutically acceptable salt thereof.

In some embodiments, the first nucleotide from the 5′ end of the sense strand includes a structure of

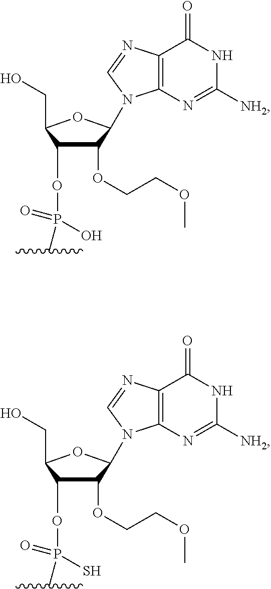

or a pharmaceutically acceptable salt thereof. In some embodiments, the first nucleotide from the 5′ end of the sense strand includes a structure of

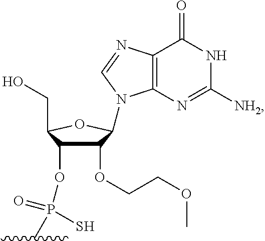

or a pharmaceutically acceptable salt thereof.

In some embodiments, the first nucleotide from the 3′ end of the sense strand has a structure of

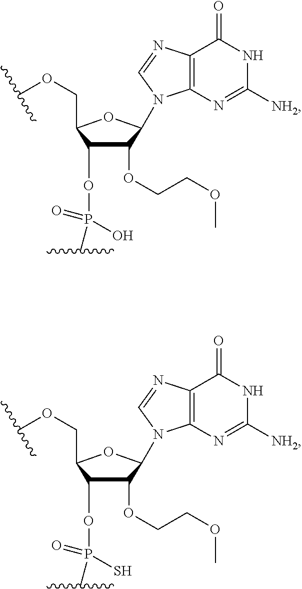

or a pharmaceutically acceptable salt thereof. In some embodiments, the first nucleotide from the 3′ end of the sense strand has a structure of

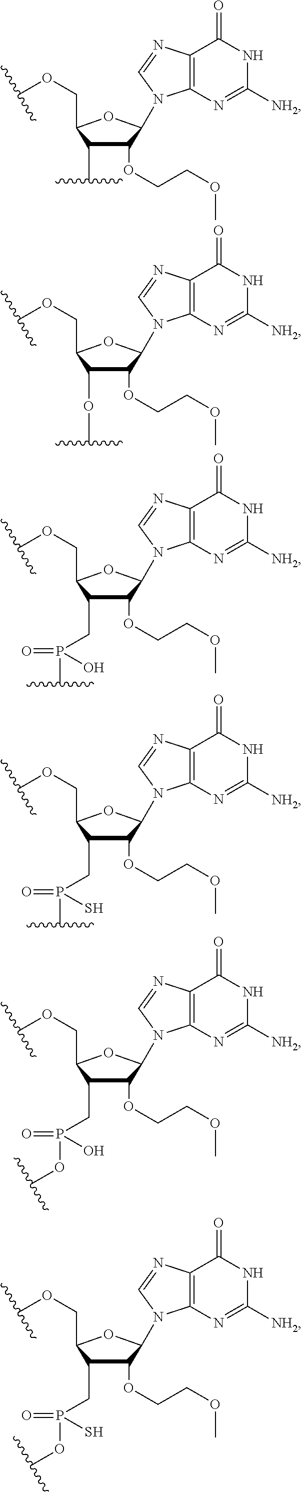

or a pharmaceutically acceptable salt thereof, wherein [symbol] is an attachment point to a ligand. In some embodiments, the first nucleotide from the 3′ end of the sense strand has a structure of

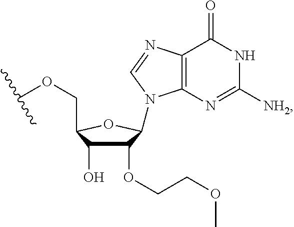

or a pharmaceutically acceptable salt thereof.

In certain aspects, the 2′-MOE modified nucleotides locate at both 5′ and 3′ ends of a sense strand so as to form a structural confinement (“2′-MOE clamp”) at the sense strand termini. In some embodiments, the 2′-MOE clamps may be symmetric and having the same number of 2′-MOE modified nucleotides at both 5′ and 3′ ends of the sense strand. For example, the sense strand includes one 2′-MOE modified nucleotide at 5′ end and one 2′-MOE modified nucleotide at 3′ end; two 2′-MOE modified nucleotides at 5′ end and two 2′-MOE modified nucleotides at 3′ end; or three 2′-MOE modified nucleotides at 5′ end and three 2′-MOE modified nucleotides at 3′ end. In some embodiments, the 2′-MOE clamps may be asymmetric and having different numbers of 2′-MOE nucleotides at 5′ and 3′ ends of the sense strand. For example, the sense strand includes one 2′-MOE modified nucleotide at 5′ end only; one 2′-MOE modified nucleotide at 3′ end only; two 2′-MOE modified nucleotides at 5′ end only; two 2′-MOE modified nucleotides at 3′ end only; one 2′-MOE modified nucleotide at 5′ end and two 2′-MOE modified nucleotides at 3′ end; or two 2′-MOE modified nucleotides at 5′ end and one 2′-MOE modified nucleotide at 3′ end.

In certain aspects, the sense strand includes one 2′-MOE modified nucleotide at 5′ end and one 2′-MOE modified nucleotide at 3′ end. In some embodiments, the sense strand includes only one 2′-MOE modified nucleotide at 5′ end and only one 2′-MOE modified nucleotide at 3′ end. In some embodiments, the sense strand includes only one 2′-MOE modified nucleotide at 5′ end. In some embodiments, the sense strand includes only one 2′-MOE modified nucleotide at 3′ end.

In certain aspects, the sense strand includes at least two contiguous 2′-MOE modified nucleotides at 5′ end and at least two 2′-MOE modified nucleotides at 3′ end. In some embodiments, the sense strand includes only two 2′-MOE modified nucleotides at 5′ end and only two 2′-MOE modified nucleotides at 3′ end. In some embodiments, the sense strand includes only two 2′-MOE modified nucleotides at 5′ end. In some embodiments, the sense strand includes only two 2′-MOE modified nucleotides at 3′ end.

In certain aspects, the sense strand includes one or two contiguous 2′-MOE modified nucleotides at 5′ end and/or one or two contiguous 2′-MOE modified nucleotides at 3′ end, where the sense strand comprises a nucleotide sequence selected from SEQ ID NOs: 761-776. In some embodiments, the nucleotide sequence of the sense strand comprises SEQ ID NO: 761. In some embodiments, the nucleotide sequence of the sense strand comprises SEQ ID NO: 762. In some embodiments, the nucleotide sequence of the sense strand comprises SEQ ID NO: 763. In some embodiments, the nucleotide sequence of the sense strand comprises SEQ ID NO: 764. In some embodiments, the nucleotide sequence of the sense strand comprises SEQ ID NO: 765. In some embodiments, the nucleotide sequence of the sense strand comprises SEQ ID NO: 766. In some embodiments, the nucleotide sequence of the sense strand comprises SEQ ID NO: 767. In some embodiments, the nucleotide sequence of the sense strand comprises SEQ ID NO: 768. In some embodiments, the nucleotide sequence of the sense strand comprises SEQ ID NO: 769. In some embodiments, the nucleotide sequence of the sense strand comprises SEQ ID NO: 770. In some embodiments, the nucleotide sequence of the sense strand comprises SEQ ID NO: 771. In some embodiments, the nucleotide sequence of the sense strand comprises SEQ ID NO: 772. In some embodiments, the nucleotide sequence of the sense strand comprises SEQ ID NO: 773. In some embodiments, the nucleotide sequence of the sense strand comprises SEQ ID NO: 774. In some embodiments, the nucleotide sequence of the sense strand comprises SEQ ID NO: 775. In some embodiments, the nucleotide sequence of the sense strand comprises SEQ ID NO: 776.

In certain aspects, the sense strand is 21 nucleotides in length. In some embodiments, the sense strand includes one, two, three, or four 2′-MOE modified nucleotides positioned at the 1st, 2nd, 20th, and/or 21st nucleotides from the 5′ end of the sense strand. In some embodiments, the sense strand includes two 2′-MOE modified nucleotides positioned at the 1st, 2nd, 20th, or 21st nucleotides from the 5′ end of the sense strand. In some embodiments, the sense strand includes three 2′-MOE modified nucleotides positioned at the 1st, 2nd, 20th, or 21st nucleotides from the 5′ end of the sense strand. In some embodiments, the sense strand includes 2′-MOE modified nucleotides positioned at the 1st, 2nd, 20th, and 21st nucleotides from the 5′ end of the sense strand. In some embodiments, the sense strand does not include a 2′-MOE modified nucleotide at the 3rd to 19th nucleotides from 5′ end of the sense strands.

Alternatively, in certain aspects, a sense strand of the dsRNA as described herein includes two or more TNAs. In some embodiments, the sense strand includes two, four, six or eight TNAs. In some embodiments, the sense strand includes two TNAs. In some embodiments, the sense strand includes four TNAs. In some embodiments, the sense strand includes six TNAs. In some embodiments, the sense strand includes eight TNAs.

In certain aspects, the sense strand includes at least two contiguous TNAs at 5′ end and at least two TNAs at 3′ end. In some embodiments, the sense strand includes only two TNAs at 5′ end and only two TNAs at 3′ end. In some embodiments, the sense strand includes only two TNAs at 5′ end. In some embodiments, the sense strand includes only two TNAs at 3′ end.

In some embodiments, the TNAs in the sense strand as described herein include a structure of

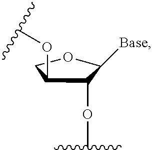

or a pharmaceutically acceptable salt thereof, wherein [symbol] is an attachment point to a linkage (e.g., phosphate or phosphorothioate group) or the adjacent nucleotides and “Base” is a nucleobase.

In some embodiments, the TNAs in the sense strand as described herein include a structure of

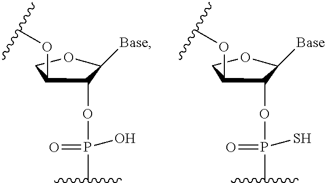

or a pharmaceutically acceptable salt thereof, wherein [symbol] is an attachment point to a terminal group (e.g., H, OH, or salt) or the adjacent nucleotides and “Base” is a nucleobase. In some embodiments, the TNAs in the dsRNA as described herein include a structure of

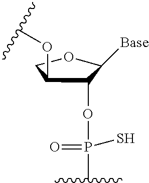

or a pharmaceutically acceptable salt thereof.

In some embodiments, the TNAs include a structure of

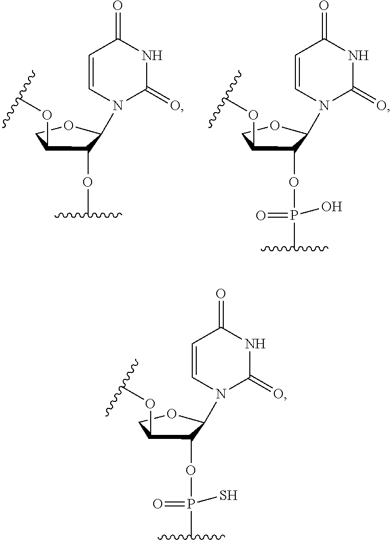

or a pharmaceutically acceptable salt thereof, wherein [symbol] is an attachment point to a terminal group (e.g., H, OH, or salt) or the adjacent nucleotides. In some embodiments, the TNAs include a structure of

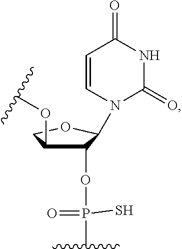

or a pharmaceutically acceptable salt thereof.

In some embodiments, the TNAs include a nucleotide having a structure of

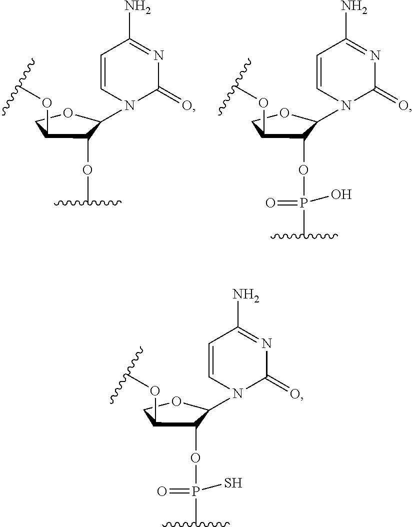

or a pharmaceutically acceptable salt thereof, wherein [symbol] is an attachment point to a terminal group (e.g., H, OH, or salt) or the adjacent nucleotides. In some embodiments, the TNAs include a nucleotide having a structure of

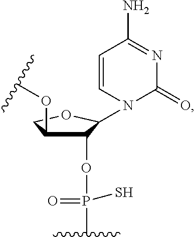

or a pharmaceutically acceptable salt thereof.

In some embodiments, the TNAs in the dsRNA as described herein include a nucleotide having a structure of

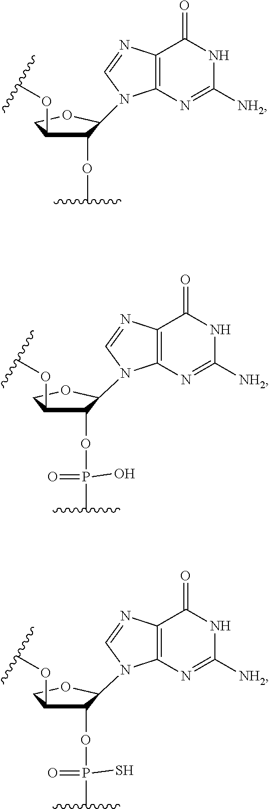

or a pharmaceutically acceptable salt thereof, wherein [symbol] is an attachment point to a terminal group (e.g., H, OH, or salt) or the adjacent nucleotides. In some embodiments, the TNAs in the dsRNA as described herein include a nucleotide having a structure of

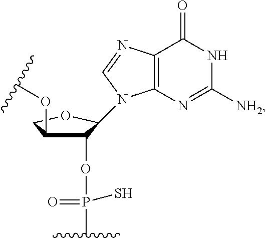

or a pharmaceutically acceptable salt thereof.

In some embodiments, the TNAs include a nucleotide having a structure of

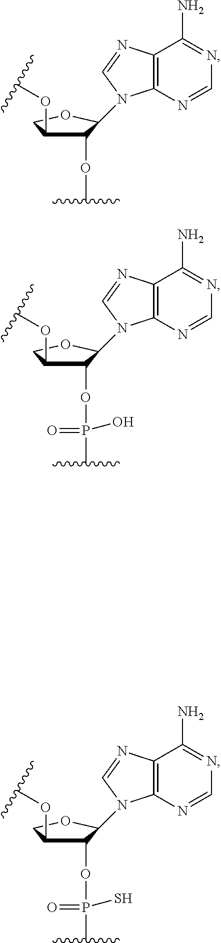

or a pharmaceutically acceptable salt thereof, wherein [symbol] is an attachment point to a terminal group (e.g., H, OH, or salt) or the adjacent nucleotides. In some embodiments, the TNAs include a nucleotide having a structure of

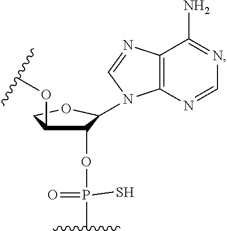

or a pharmaceutically acceptable salt thereof.

In certain aspects, at least one of the TNAs in the sense strand as described herein has a structure of

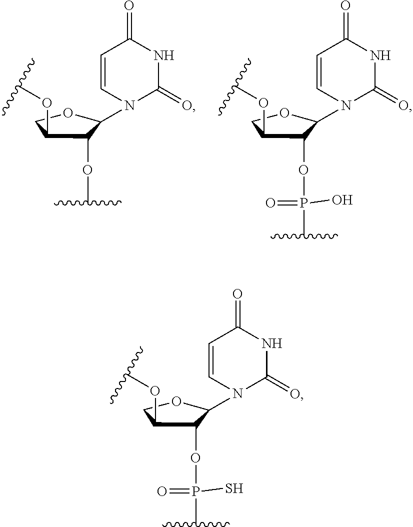

or a pharmaceutically acceptable salt thereof, wherein [symbol] is an attachment point to a terminal group (e.g., H, OH, or salt) or the adjacent nucleotides. In some embodiments, at least one of the TNAs in the sense strand as described herein has a structure of

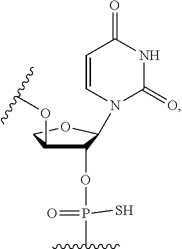

or a pharmaceutically acceptable salt thereof.

In some embodiments, the first nucleotide from the 5′ end of the sense strand includes a structure of

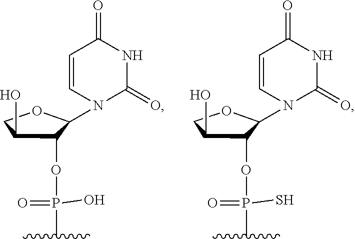

or a pharmaceutically acceptable salt thereof. In some embodiments, the first nucleotide from the 5′ end of the sense strand includes a structure of

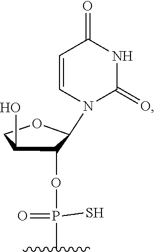

or a pharmaceutically acceptable salt thereof.

In some embodiments, the first nucleotide from the 3′ end of the sense strand includes a structure of

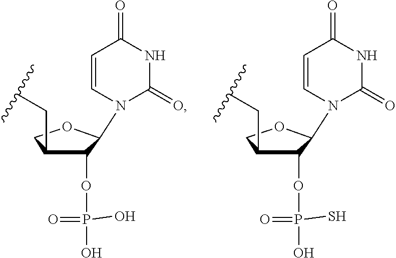

or a pharmaceutically acceptable salt thereof. In some embodiments, the first nucleotide from the 3′ end of the sense strand includes a structure of

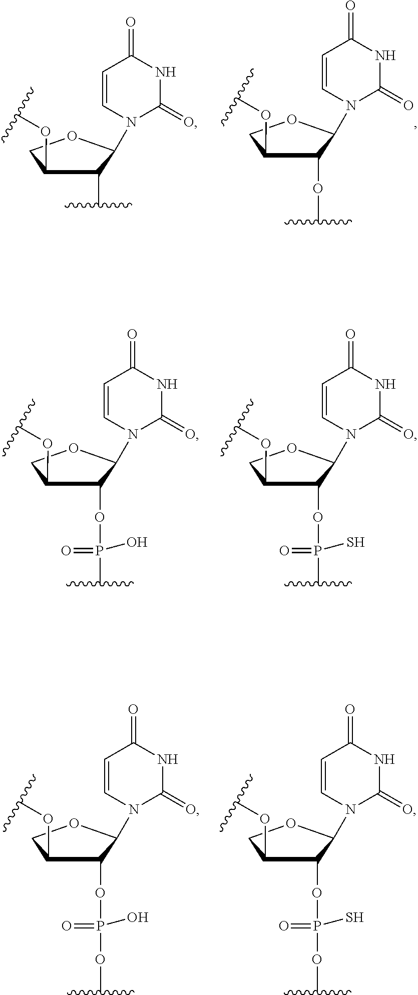

or a pharmaceutically acceptable salt thereof and [symbol] is an attachment point to a ligand. In some embodiments, the first nucleotide from the 3′ end of the sense strand includes a structure of

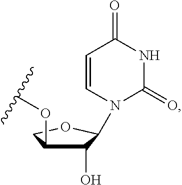

or a pharmaceutically acceptable salt thereof.

In certain aspects, at least one of the TNAs in the sense strand as described herein has a structure of

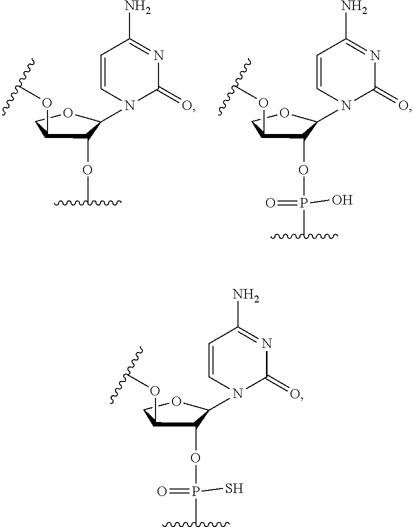

or a pharmaceutically acceptable salt thereof, wherein [symbol] is an attachment point to a terminal group (e.g., H, OH, or salt) or the adjacent nucleotides. In some embodiments, at least one of the TNAs in the sense strand as described herein has a structure of

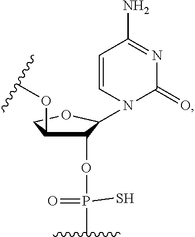

or a pharmaceutically acceptable salt thereof.

In some embodiments, the first nucleotide from the 5′ end of the sense strand includes a structure of

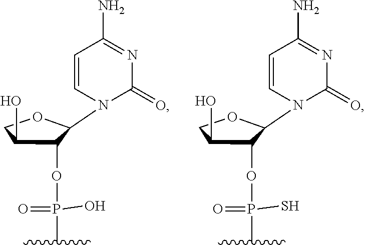

or a pharmaceutically acceptable salt thereof. In some embodiments, the first nucleotide from the 5′ end of the sense strand includes a structure of

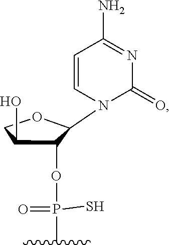

or a pharmaceutically acceptable salt thereof.

In some embodiments, the first nucleotide from the 3′ end of the sense strand includes a structure of

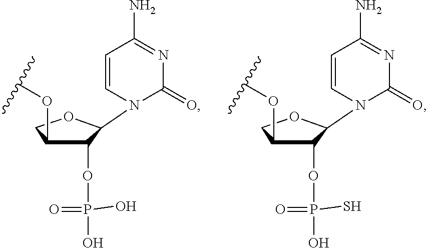

or a pharmaceutically acceptable salt thereof. In some embodiments, the first nucleotide from the 3′ end of the sense strand includes a structure of

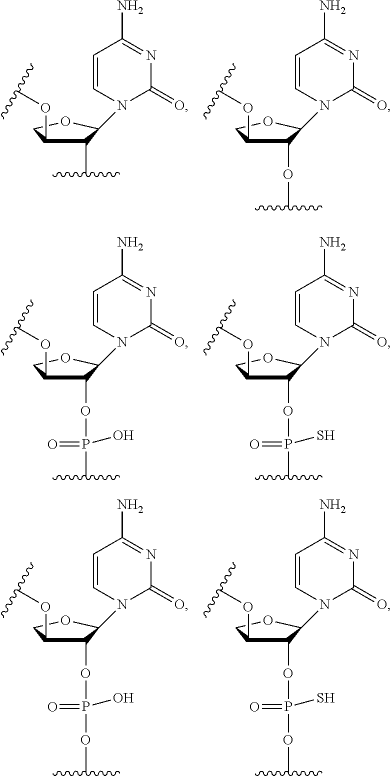

or a pharmaceutically acceptable salt thereof and [symbol] is an attachment point to a ligand. In some embodiments, the first nucleotide from the 3′ end of the sense strand includes a structure of

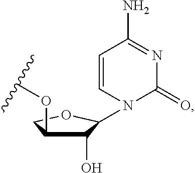

or a pharmaceutically acceptable salt thereof.

In certain aspects, at least one of the TNAs in the sense strand as described herein has a structure of

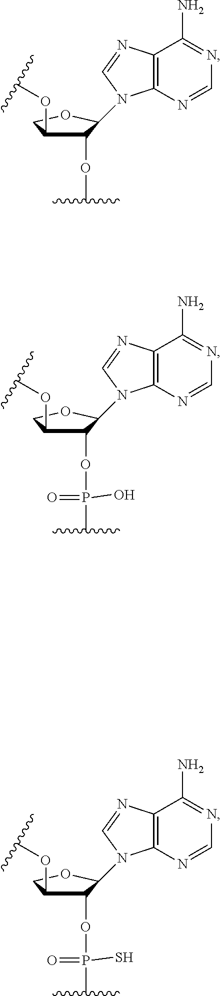

or a pharmaceutically acceptable salt thereof, wherein [symbol] is an attachment point to a terminal group (e.g., H, OH, or salt) or the adjacent nucleotides. In some embodiments, at least one of the TNAs in the sense strand as described herein has a structure of

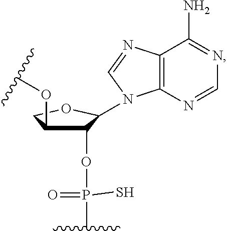

or a pharmaceutically acceptable salt thereof.

In some embodiments, the first nucleotide from the 5′ end of the sense strand includes a structure of

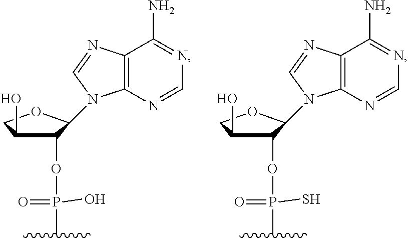

or a pharmaceutically acceptable salt thereof. In some embodiments, the first nucleotide from the 5′ end of the sense strand includes a structure of

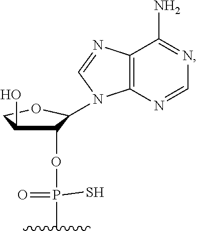

or a pharmaceutically acceptable salt thereof.

In some embodiments, the first nucleotide from the 3′ end of the sense strand includes a structure of

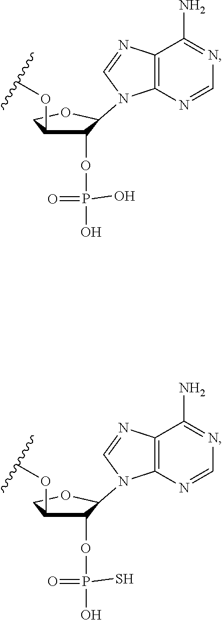

or a pharmaceutically acceptable salt thereof. In some embodiments, the first nucleotide from the 3′ end of the sense strand includes a structure of

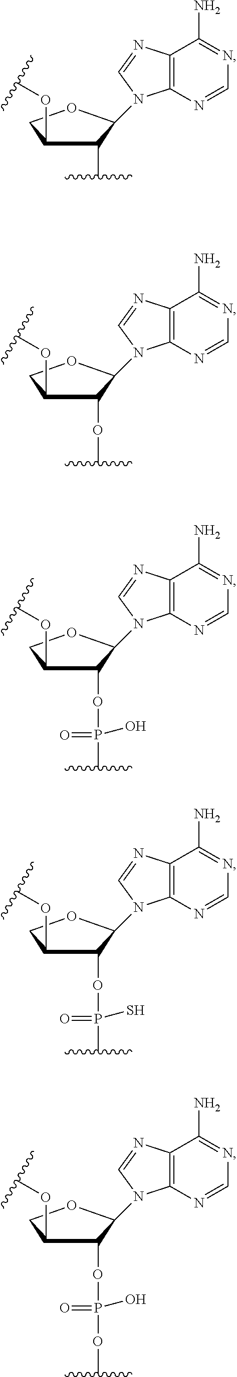

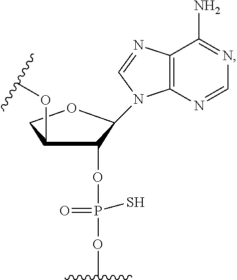

or a pharmaceutically acceptable salt thereof and [symbol] is an attachment point to a ligand. In some embodiments, the first nucleotide from the 3′ end of the sense strand includes a structure of

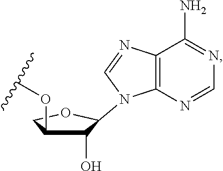

or a pharmaceutically acceptable salt thereof.

In certain aspects, at least one of the TNAs in the sense strand as described herein has a structure of

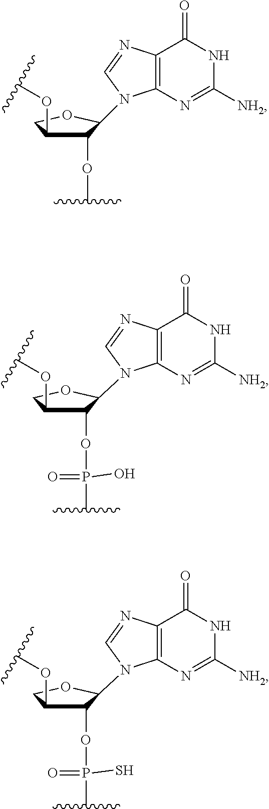

or a pharmaceutically acceptable salt thereof, wherein [symbol] is an attachment point to a terminal group (e.g., H, OH, or salt) or the adjacent nucleotides. In some embodiments, at least one of the TNAs in the sense strand as described herein has a structure of

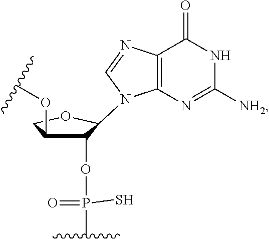

or a pharmaceutically acceptable salt thereof.

In some embodiments, the first nucleotide from the 5′ end of the sense strand includes a structure of

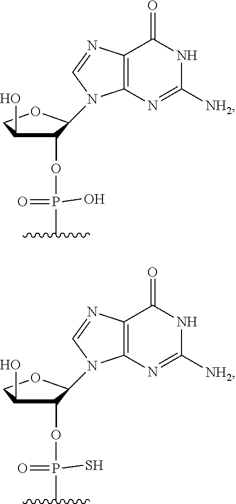

or a pharmaceutically acceptable salt thereof. In some embodiments, the first nucleotide from the 5′ end of the sense strand includes a structure of

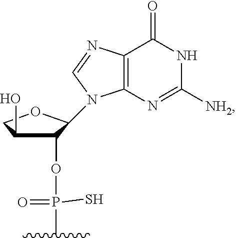

or a pharmaceutically acceptable salt thereof.

In some embodiments, the first nucleotide from the 3′ end of the sense strand includes a structure of

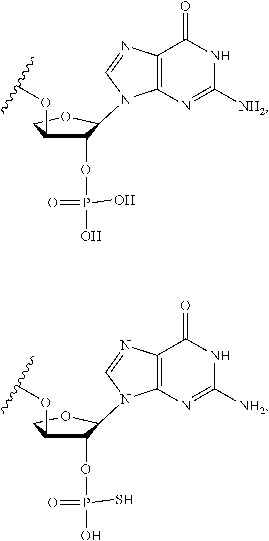

or a pharmaceutically acceptable salt thereof. In some embodiments, the first nucleotide from the 3′ end of the sense strand includes a structure of

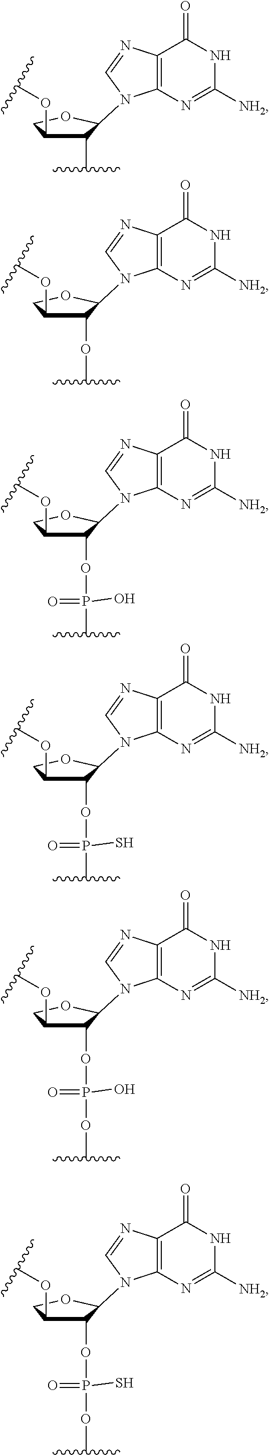

or a pharmaceutically acceptable salt thereof and [symbol] is an attachment point to a ligand. In some embodiments, the first nucleotide from the 3′ end of the sense strand includes a structure of

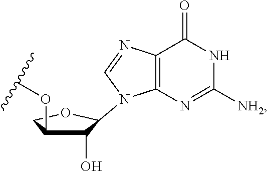

or a pharmaceutically acceptable salt thereof.

In certain aspects, the TNAs locate at both 5′ and 3′ ends of a sense strand so as to form a structural confinement (“TNA clamp”) at the sense strand termini. In some embodiments, the TNA clamps may be symmetric and having the same number of TNAs at both 5′ and 3′ ends of the sense strand. For example, the sense strand includes one TNA at 5′ end and one TNA at 3′ end; two TNAs at 5′ end and two TNAs at 3′ end; or three TNAs at 5′ end and three TNAs at 3′ end. In some embodiments, the TNA clamps may be asymmetric and having different numbers of TNAs at 5′ and 3′ ends of the sense strand. For example, the sense strand includes one TNA at 5′ end only; one TNA at 3′ end only; two TNAs at 5′ end only; two TNAs at 3′ end only; one TNA at 5′ end and two TNAs at 3′ end; or two TNAs at 5′ end and one TNA at 3′ end.

In certain aspects, the sense strand includes one TNA at 5′ end and one TNA at 3′ end. In some embodiments, the sense strand includes only one TNA at 5′ end and only one TNA at 3′ end. In some embodiments, the sense strand includes only one TNA at 5′ end. In some embodiments, the sense strand includes only one TNA at 3′ end.

In certain aspects, the sense strand includes at least two contiguous TNAs at 5′ end and at least two TNAs at 3′ end. In some embodiments, the sense strand includes only two TNAs at 5′ end and only two TNAs at 3′ end. In some embodiments, the sense strand includes only two TNAs at 5′ end. In some embodiments, the sense strand includes only two TNAs at 3′ end.

In certain aspects, the sense strand includes one or two contiguous 2′-TNA modified nucleotides at 5′ end and/or one or two contiguous 2′-TNA modified nucleotides at 3′ end, where the sense strand comprises a nucleotide sequence selected from SEQ ID NOs: 761-776. In some embodiments, the nucleotide sequence of the sense strand comprises SEQ ID NO: 761. In some embodiments, the nucleotide sequence of the sense strand comprises SEQ ID NO: 762. In some embodiments, the nucleotide sequence of the sense strand comprises SEQ ID NO: 763. In some embodiments, the nucleotide sequence of the sense strand comprises SEQ ID NO: 764. In some embodiments, the nucleotide sequence of the sense strand comprises SEQ ID NO: 765. In some embodiments, the nucleotide sequence of the sense strand comprises SEQ ID NO: 766. In some embodiments, the nucleotide sequence of the sense strand comprises SEQ ID NO: 767. In some embodiments, the nucleotide sequence of the sense strand comprises SEQ ID NO: 768. In some embodiments, the nucleotide sequence of the sense strand comprises SEQ ID NO: 769. In some embodiments, the nucleotide sequence of the sense strand comprises SEQ ID NO: 770. In some embodiments, the nucleotide sequence of the sense strand comprises SEQ ID NO: 771. In some embodiments, the nucleotide sequence of the sense strand comprises SEQ ID NO: 772. In some embodiments, the nucleotide sequence of the sense strand comprises SEQ ID NO: 773. In some embodiments, the nucleotide sequence of the sense strand comprises SEQ ID NO: 774. In some embodiments, the nucleotide sequence of the sense strand comprises SEQ ID NO: 775. In some embodiments, the nucleotide sequence of the sense strand comprises SEQ ID NO: 776.

In certain aspects, the sense strand is 21 nucleotides in length. In some embodiments, the sense strand includes one, two, three, or four TNAs positioned at the 1st, 2nd, 20th, and/or 21st nucleotides from the 5′ end of the sense strand. In some embodiments, the sense strand includes two TNAs positioned at the 1st, 2nd, 20th, or 21st nucleotides from the 5′ end of the sense strand. In some embodiments, the sense strand includes three TNAs positioned at the 1st, 2nd, 20th, or 21st nucleotides from the 5′ end of the sense strand. In some embodiments, the sense strand includes TNAs positioned at the 1st, 2nd, 20th, and 21st nucleotides from the 5′ end of the sense strand. In some embodiments, the sense strand does not include a TNA at the 3rd to 19th nucleotides from 5′ end of the sense strands.

In certain aspects, the sense strand of the dsRNA as described herein includes two or more 2′-F modifications. In some embodiments, the sense strand of the dsRNA includes two, three, four, five, six, seven, or eight 2′-F modified nucleotides. In some embodiments, the sense strand includes two 2′-F modified nucleotides. In some embodiments, the sense strand includes three 2′-F modified nucleotides. In some embodiments, the sense strand includes four 2′-F modified nucleotides. In some embodiments, the sense strand includes five 2′-F modified nucleotides. In some embodiments, the sense strand includes six 2′-F modified nucleotides. In some embodiments, the sense strand includes seven 2′-F modified nucleotides. In some embodiments, the sense strand includes eight 2′-F modified nucleotides. In some embodiments, two contiguous 2′-F modified nucleotides locate in the sense strand. In some embodiments, three contiguous 2′-F modified nucleotides locate in the sense strand. In some embodiments, four contiguous 2′-F modified nucleotides locate in the sense strand.

In certain aspects, the sense strand is 21 nucleotides in length. In some embodiments, 2′-F modified nucleotides locate at 5th, 7th, 8th, and/or 9th positions from the 5′ end of the sense strand. In some embodiments, 2′-F modified nucleotides locate at 6th, 8th, 9th, and/or 10th positions from the 5′ end of the sense strand. In some embodiments, 2′-F modified nucleotides locate at 7th, 9th, 10th, and/or 11th positions from the 5′ end of the sense strand. In some embodiments, 2′-F modified nucleotides locate at 8th, 10th, 11th, and/or 12th positions from the 5′ end of the sense strand. In some embodiments, 2′-F modified nucleotides locate at 9th, 11th, 12th, and/or 13th positions from the 5′ end of the sense strand.

In certain aspects, a sense strand of the dsRNA as described herein includes one, two, three, or four 2′-deoxy modifications (DNA). In some embodiments, the sense strand of the dsRNA includes one 2′-deoxy modified nucleotide. In some embodiments, the sense strand includes two 2′-deoxy modified nucleotides. In some embodiments, the sense strand includes three 2′-deoxy modified nucleotides. In some embodiments, the sense strand includes four 2′-deoxy modified nucleotides.

In certain aspects, a sense strand of the dsRNA as described herein includes one, two, three, or four deoxythymidines (dT). In some embodiments, the sense strand of the dsRNA includes one deoxythymidine (dT). In some embodiments, the sense strand includes two deoxythymidines. In some embodiments, the sense strand includes three deoxythymidines (dT). In some embodiments, the sense strand includes four deoxythymidines.

In some embodiments, the sense strand is 21 nucleotides in length. In some embodiments, one or more 2′-deoxy modified nucleotides locate at 5th, 7th, 8th, and/or 9th positions from the 5′ end of the sense strand. In some embodiments, one or more 2′-deoxy modified nucleotides locate at 6th, 8th, 9th, and/or 10th positions from the 5′ end of the sense strand. In some embodiments, one or more 2′-deoxy modified nucleotides locate at 7th, 9th, 10th, and/or 11th positions from the 5′ end of the sense strand. In some embodiments, one or more 2′-deoxy modified nucleotides locate at 8th, 10th, 11th, and/or 12th positions from the 5′ end of the sense strand. In some embodiments, one or more 2′-deoxy modified nucleotides locate at 9th, 11th, 12th, and/or 13th positions from the 5′ end of the sense strand.

In some embodiments, the sense strand is 21 nucleotides in length and one 2′-deoxy modified nucleotide locates at 7th position from the 5′ end of the sense strand. In some embodiments, the sense strand is 21 nucleotides in length and one 2′-deoxy modified nucleotide locates at 8th position from the 5′ end of the sense strand. In some embodiments, the sense strand is 21 nucleotides in length and one 2′-deoxy modified nucleotide locates at 9th position from the 5′ end of the sense strand. In some embodiments, the sense strand is 21 nucleotides in length and one 2′-deoxy modified nucleotide locates at 10th position from the 5′ end of the sense strand. In some embodiments, the sense strand is 21 nucleotides in length and one 2′-deoxy modified nucleotide locates at 11th position from the 5′ end of the sense strand. In some embodiments, the sense strand is 21 nucleotides in length and one 2′-deoxy modified nucleotide locates at 12th position from the 5′ end of the sense strand. In some embodiments, the sense strand is 21 nucleotides in length and one 2′-deoxy modified nucleotide locates at 13th position from the 5′ end of the sense strand. In some embodiments, the sense strand is 21 nucleotides in length and one 2′-deoxy modified nucleotide locates at 14th position from the 5′ end of the sense strand.

In some embodiments, the sense strand is 21 nucleotides in length and one deoxythymidine (dT) locates at 7th position from the 5′ end of the sense strand. In some embodiments, the sense strand is 21 nucleotides in length and one deoxythymidine (dT) locates at 8th position from the 5′ end of the sense strand. In some embodiments, the sense strand is 21 nucleotides in length and one deoxythymidine (dT) locates at 9th position from the 5′ end of the sense strand. In some embodiments, the sense strand is 21 nucleotides in length and one deoxythymidine (dT) locates at 10th position from the 5′ end of the sense strand. In some embodiments, the sense strand is 21 nucleotides in length and one deoxythymidine (dT) locates at 11th position from the 5′ end of the sense strand. In some embodiments, the sense strand is 21 nucleotides in length and one deoxythymidine (dT) locates at 12th position from the 5′ end of the sense strand. In some embodiments, the sense strand is 21 nucleotides in length and one deoxythymidine (dT) locates at 13th position from the 5′ end of the sense strand. In some embodiments, the sense strand is 21 nucleotides in length and one deoxythymidine (dT) locates at 14th position from the 5′ end of the sense strand.

In some embodiments, when the sense strand is 21 nucleotides in length, 2′-F modified nucleotides locate at 5th, 7th, and 8th from the 5′ end of the sense strand and one deoxythymidine (dT) locates at 9th position from the 5′ end of the sense strand. In some embodiments, when the sense strand is 21 nucleotides in length, 2′-F modified nucleotides locate at 6th, 8th, and 9th from the 5′ end of the sense strand and one deoxythymidine (dT) locates at 10th position from the 5′ end of the sense strand. In some embodiments, when the sense strand is 21 nucleotides in length, 2′-F modified nucleotides locate at 7th, 9th, and 10th from the 5′ end of the sense strand and one deoxythymidine (dT) locates at 11th position from the 5′ end of the sense strand. In some embodiments, when the sense strand is 21 nucleotides in length, 2′-F modified nucleotides locate at 8th, 10th, and 11th from the 5′ end of the sense strand and one deoxythymidine (dT) locates at 12th position from the 5′ end of the sense strand. In some embodiments, when the sense strand is 21 nucleotides in length, 2′-F modified nucleotides locate at 9th, 11th, and 12th from the 5′ end of the sense strand and one deoxythymidine (dT) locates at 13th position from the 5′ end of the sense strand.

In some embodiments, the sense strand includes 2′-OMe modified nucleotides in the remaining positions in the sense strand.

In certain aspects, the sense strand includes one to six phosphorothioate (PS) linkages between nucleosides. In some embodiments, the sense strand includes one, two, three, or four phosphorothioate (PS) linkages between nucleosides.

In certain aspects, the sense strand is 21 nucleotides in length. In some embodiments, the sense strand includes two 3′-PS modified nucleotides at the 1st, 2nd, 19th and/or 20th positions from 5′ end of the sense strand. In some embodiments, the sense strand includes three 3′-PS modified nucleotides at the 1st, 2nd, 19th and/or 20th positions from 5′ end of the sense strand. In some embodiments, the sense strand includes 3′-PS modified nucleotides at the 1st, 2nd, 19th and 20th positions from 5′ end of the sense strand.

In certain aspects, the sense strand is 21 nucleotides in length. In some embodiments, the sense strand includes 3′-PS modified nucleotides at the 1st and 2nd positions from 5′ end of the sense strand. In some embodiments, the sense strand includes a 3′-PS modified nucleotide at the 19th and 20th position from 5′ end of the sense strand. In some embodiments, the sense strand includes 3′-PS modified nucleotides at the 1st and 20th positions from 5′ end of the sense strand. In some embodiments, the sense strand includes 3′-PS modified nucleotides at the 1st, 2nd, 19th and 20th positions from 5′ end of the sense strand.

In certain aspects, the sense strand includes two to eight phosphorothioate (PS) groups or linkages between nucleosides. In certain aspects, the sense strand is 21 nucleotides in length. In some embodiments, the sense strand includes two 3′-PS modified nucleotides positioned at the 1st, 2nd, 3rd, 4th, 17th, 18th, 19th and/or 20th nucleotides from 5′ end of the sense strand. In some embodiments, the sense strand includes four 3′-PS modified nucleotides positioned at the 1st, 2nd, 3rd, 4th, 17th, 18th, 19th and/or 20th nucleotides from 5′ end of the sense strand. In some embodiments, the sense strand includes six 3′-PS modified nucleotides positioned at the 1st, 2nd, 3rd, 4th, 17th, 18th, 19th and/or 20th nucleotides from 5′ end of the sense strand. In some embodiments, the sense strand includes 3′-PS modified nucleotides positioned at the 1st, 2nd, 3rd, 4th, 17th, 18th, 19th and 20th nucleotides from 5′ end of the sense strand.

In certain aspects, the sense strand is 21 nucleotides in length. In some embodiments, at least one of the 3′-PS groups at the 1st, 2nd, 3rd, 4th, 17th, 18th, 19th and/or 20th nucleotides from 5′ end of the sense strand is a stereopure Rp isomer. In some embodiments, at least one of the 3′-PS groups at the 1st, 2nd, 19th and/or 20th nucleotides from 5′ end of the sense strand is a stereopure Rp isomer. In some embodiments, at least one of the 3′-PS groups at the 1st and/or 20th nucleotides from 5′ end of the sense strand is a stereopure Rp isomer. In some embodiments, the 3′-PS group at the 1st nucleotide from 5′ end of the sense strand is a stereopure Rp isomer. In some embodiments, the 3′-PS group at the 2nd nucleotide from 5′ end of the sense strand is a stereopure Rp isomer. In some embodiments, the 3′-PS group at the 19th nucleotide from 5′ end of the sense strand is a stereopure Rp isomer. In some embodiments, the 3′-PS group at the 20th nucleotide from 5′ end of the sense strand is a stereopure Rp isomer. In some embodiments, the 3′-PS groups at the 1st and 20th nucleotides from 5′ end of the sense strand are stereopure Rp isomers. In some embodiments, the 3′-PS groups at the 1st, 2nd, 19^{th} and 20th nucleotides from 5′ end of the sense strand are stereopure Rp isomers.

In certain aspects, the sense strand is 21 nucleotides in length. In some embodiments, at least one of the 3′-PS groups at the 1st, 2nd, 3rd, 4th, 17th, 18th, 19th and/or 20th nucleotides from 5′ end of the sense strand is a stereopure Sp isomer. In some embodiments, at least one of the 3′-PS groups at the 1st, 2nd, 19th and/or 20th nucleotides from 5′ end of the sense strand is a stereopure Sp isomer. In some embodiments, at least one of the 3′-PS groups at the 1st and/or 20th nucleotides from 5′ end of the sense strand is a stereopure Sp isomer. In some embodiments, the 3′-PS group at the 1st nucleotide from 5′ end of the sense strand is a stereopure Sp isomer. In some embodiments, the 3′-PS group at the 2nd nucleotide from 5′ end of the sense strand is a stereopure Sp isomer. In some embodiments, the 3′-PS group at the 19th nucleotide from 5′ end of the sense strand is a stereopure Sp isomer. In some embodiments, the 3′-PS group at the 20nd nucleotide from 5′ end of the sense strand is a stereopure Sp isomer. In some embodiments, the 3′-PS groups at the 1st and 20th nucleotides from 5′ end of the sense strand are stereopure Sp isomers. In some embodiments, the 3′-PS groups at the 1st, 2nd, 19th, and 20th nucleotides from 5′ end of the sense strand are stereopure Sp isomers.

In certain aspects, a sense strand of the dsRNA as described herein includes one or more TNAs, one or more 2′-F modified nucleotides, one or more 2′-deoxy modified nucleotides, and one or more 2′-OMe modified nucleotides. In certain aspects, a sense strand of the dsRNA as described herein consists of one or more TNAs, one or more 2′-F modified nucleotides, one or more 2′-deoxy modified nucleotides, and one or more 2′-OMe modified nucleotides.

In certain aspects, a sense strand of the dsRNA as described herein may have a Formula (I)

(I)
5′-X₁-X₂-X₃-X₄-X₅-X₆-X₇-X₈-X₉-X₁₀-X₁₁-X₁₂-X₁₃-
X₁₄-X₁₅-X₁₆-X₁₇-X₁₈-X₁₉-X₂₀-X₂₁-3′

wherein:
each X₁, X₂, X₂₀, and X₂₁ is independently a 2′-MOE modified nucleotide;
each X₃ to X₁₉ is independently selected from a 2′-deoxy modified nucleotide, 2′-MOE modified nucleotide, 2′-F modified nucleotide, and 2′-OMe modified nucleotide.

In some embodiments, the nucleotide sequence of the sense strand of Formula (I) comprises a sequence selected from the SEQ ID NOs: 761-776. In some embodiments, the nucleotide sequence of the sense strand of Formula (I) comprises a sequence SEQ ID NO: 761. In some embodiments, the nucleotide sequence of the sense strand of Formula (I) comprises a sequence SEQ ID NO: 762. In some embodiments, the nucleotide sequence of the sense strand of Formula (I) comprises a sequence SEQ ID NO: 763. In some embodiments, the nucleotide sequence of the sense strand of Formula (I) comprises a sequence SEQ ID NO: 764. In some embodiments, the nucleotide sequence of the sense strand of Formula (I) comprises a sequence SEQ ID NO: 765. In some embodiments, the nucleotide sequence of the sense strand of Formula (I) comprises a sequence SEQ ID NO: 766. In some embodiments, the nucleotide sequence of the sense strand of Formula (I) comprises a sequence SEQ ID NO: 767. In some embodiments, the nucleotide sequence of the sense strand of Formula (I) comprises a sequence SEQ ID NO: 768. In some embodiments, the nucleotide sequence of the sense strand of Formula (I) comprises a sequence SEQ ID NO: 769. In some embodiments, the nucleotide sequence of the sense strand of Formula (I) comprises a sequence SEQ ID NO: 770. In some embodiments, the nucleotide sequence of the sense strand of Formula (I) comprises a sequence SEQ ID NO: 771. In some embodiments, the nucleotide sequence of the sense strand of Formula (I) comprises a sequence SEQ ID NO: 772. In some embodiments, the nucleotide sequence of the sense strand of Formula (I) comprises a sequence SEQ ID NO: 773. In some embodiments, the nucleotide sequence of the sense strand of Formula (I) comprises a sequence SEQ ID NO: 774. In some embodiments, the nucleotide sequence of the sense strand of Formula (I) comprises a sequence SEQ ID NO: 775. In some embodiments, the nucleotide sequence of the sense strand of Formula (I) comprises a sequence SEQ ID NO: 776.

In some embodiments, the first nucleotide from the 5′ end of the sense strand (X₁) of Formula (I) is a 2′-MOE modified nucleotide with a nucleobase T. In some embodiments, the first nucleotide from the 5′ end of the sense strand (X₁) is a 2′-MOE modified nucleotide with a nucleobase methylated cytosine (e.g., 5-methylcytosine or N⁴-methylcytosine). In some embodiments, the second nucleotide from the 5′ end of the sense strand (X₂) is a 2′-MOE modified nucleotide with a nucleobase A, T, G or methylated cytosine (e.g., 5-methylcytosine or N⁴-methylcytosine). In some embodiments, the second nucleotide from the 5′ end of the sense strand (X₂) is a 2′-MOE modified nucleotide with a nucleobase T. In some embodiments, the second nucleotide from the 5′ end of the sense strand (X₂) is a 2′-MOE modified nucleotide with a nucleobase G. In some embodiments, the second nucleotide from the 5′ end of the sense strand (X₂) is a 2′-MOE modified nucleotide with a nucleobase A. In some embodiments, the second nucleotide from the 5′ end of the sense strand (X₂) is a 2′-MOE modified nucleotide with a nucleobase methylated cytosine (e.g., 5-methylcytosine or N⁴-methylcytosine).

In some embodiments, the first nucleotide from the 3′ end of the sense strand (X₂₁) is a 2′-MOE modified nucleotide with a nucleobase A or T. In some embodiments, the first nucleotide from the 3′ end of the sense strand (X₂₁) is a 2′-MOE modified nucleotide with a nucleobase A. In some embodiments, the first nucleotide from the 3′ end of the sense strand (X₂₁) is a 2′-MOE modified nucleotide with a nucleobase T. In some embodiments, the second nucleotide from the 3′ end of the sense strand (X₂₀) is a 2′-MOE modified nucleotide with a nucleobase A. In some embodiments, the second nucleotide from the 3′ end of the sense strand (X₂₀) is a 2′-MOE modified nucleotide with a nucleobase G. In some embodiments, the second nucleotide from the 3′ end of the sense strand (X₂₀) is a 2′-MOE modified nucleotide with a nucleobase T. In some embodiments, the second nucleotide from the 3′ end of the sense strand (X₂₀) is a 2′-MOE modified nucleotide with a nucleobase methylated cytosine (e.g., 5-methylcytosine or N⁴-methylcytosine).

In certain aspects, in Formula (I), X₃ to X₁₉ do not include a 2′-MOE modified nucleotide. In certain aspects, X₃ to X₁₉ do not include a TNA. In some embodiments, each X₃ to X₁₉ is selected from deoxyribonucleotide, 2′-F modified nucleotides and 2′-OMe modified nucleotides. In some embodiments, at least one of X₃ to X₁₉ is not a deoxyribonucleotide.

In some embodiments, each X_{f}, X_{f+2}, and X_{f+3} is 2′-F modified nucleotide and X_{f+4} is 2′-deoxy modified nucleotide when f is an integer from 3 to 17. In some embodiments, f is 5. In some embodiments, f is 6. In some embodiments, f is 7. In some embodiments, f is 8. In some embodiments, f is 9. In some embodiments, X₅, X₇, and X₈ are 2′-F modified nucleotides, and X₉ is 2′-deoxy modified nucleotide (e.g., dT). In some embodiments, X₆, X₈, and X₉ are 2′-F modified nucleotides and X₁₀ is 2′-deoxy modified nucleotide (e.g., dT). In some embodiments, X₇, X₉, and X₁₀ are 2′-F modified nucleotides, and X₁₁ is 2′-deoxy modified nucleotide (e.g., dT). In some embodiments, X₈, X₁₀, and X₁₁ are 2′-F modified nucleotides, and X₁₂ is 2′-deoxy modified nucleotide (e.g., dT). In some embodiments, X₉, X₁₁, and X₁₂ are 2′-F modified nucleotides, and X₁₃ is 2′-deoxy modified nucleotide (e.g., dT).

In some embodiments, the sense strand includes 2′-OMe modified nucleotides in the remaining positions in the sense strand.

In some embodiments, at least two nucleotides from X₁, X₂, X₁₉, and X₂₀ contain a 3′-PS group, respectively. In some embodiments, two nucleotides from X₁, X₂, X₁₉, and X₂₀ contain a 3′-PS group, respectively. In some embodiments, three nucleotides from X₁, X₂, X₁₉, and X₂₀ contain a 3′-PS group, respectively. In some embodiments, each X₁, X₂, X₁₉, and X₂₀ contains a 3′-PS group. In some embodiments, each X₁ and X₂ contains a 3′-PS group.

In some embodiments, at least four from X₁, X₂, X₃, X₄, X₁₇, X₁₈, X₁₉, and/or X₂₀ contain 3′-PS groups. In some embodiments, four from X₁, X₂, X₃, X₄, X₁₇, X₁₈, X₁₉, and/or X₂₀ contain a 3′-PS group, respectively. In some embodiments, six from X₁, X₂, X₃, X₄, X₁₇, X₁₈, X₁₉, and/or X₂₀ contain a 3′-PS group, respectively. In some embodiments, X₁, X₂, X₃, X₄, X₁₇, X₁₈, X₁₉, and X₂₀ contain a 3′-PS group, respectively.

In some embodiments, in X₃ to X₁₈, two to six nucleotides contain 3′-PS groups. In some embodiments, in X₃ to X₁₈, two nucleotides contain a 3′-PS group, respectively, respectively. In some embodiments, in X₃ to X₁₈, three nucleotides contain a 3′-PS group, respectively. In some embodiments, in X₃ to X₁₈, four nucleotides contain a 3′-PS group, respectively. In some embodiments, in X₃ to X₁₈, five nucleotides contain a 3′-PS group, respectively. In some embodiments, in X₃ to X₁₈, six nucleotides contain a 3′-PS group, respectively.

In certain aspects, the sense strand includes 2′-MOE modified nucleotides positioned at the 1st, 2nd, 20th, and 21st nucleotides from the 5′ end of the sense strand.

In some embodiments, the sense strand having 21 nucleotides in length includes:
(i) 2′-MOE modifications at the 1st, 2nd, 20th and/or 21st nucleotides from the 5′ end of the sense strand; and
(ii) 3′-PS modifications at the 1st, 2nd, 19th, and/or 20th nucleotides from 5′ end of the sense strand.

In some embodiments, the sense strand having 21 nucleotides in length includes:
(i) 2′-MOE modifications at the 1st, 2nd, 20th and/or 21st nucleotides from the 5′ end of the sense strand; 2′-F modifications at 7th, 9th, and 10th nucleotides from the 5′ end of the sense strand; 2′-deoxy modification at 11th nucleotide; and 2′-OMe modifications in the remaining nucleotides.

In some embodiments, the sense strand having 21 nucleotides in length includes:
(i) 2′-MOE modifications at the 1st, 2nd, 20th and/or 21st nucleotides from the 5′ end of the sense strand; 2′-F modifications at 7th, 9th, and 10th nucleotides from the 5′ end of the sense strand; and 2′-deoxy modification at 11th nucleotide; and
(ii) 3′-PS modifications at the 1st and 2nd nucleotides from 5′ end of the sense strand.

In some embodiments, the sense strand having 21 nucleotides in length includes:
(i) 2′-MOE modifications at the 1st, 2nd, 20th and/or 21st nucleotides from the 5′ end of the sense strand; 2′-F modifications at 7th, 9th, and 10th nucleotides from the 5′ end of the sense strand; and 2′-deoxy modification at 11th nucleotide; and
(ii) 3′-PS modifications at the 1st, 2nd, 19th, and 20th nucleotides from 5′ end of the sense strand.

In some embodiments, the sense strand having 21 nucleotides in length includes:
(i) 2′-MOE modifications at the 1st, 2nd, 20th and/or 21st nucleotides from the 5′ end of the sense strand; 2′-F modifications at 7th, 9, and 10th nucleotides from the 5′ end of the sense strand; 2′-deoxy modification at 11th nucleotide; and 2′-OMe modifications in the remaining nucleotides; and
(ii) 3′-PS modifications at the 1st and 2nd nucleotides from 5′ end of the sense strand.

In some embodiments, the sense strand having 21 nucleotides in length includes:
(i) 2′-MOE modifications at the 1st, 2nd, 20th and/or 21st nucleotides from the 5′ end of the sense strand; 2′-F modifications at 7th, 9, and 10th nucleotides from the 5′ end of the sense strand; 2′-deoxy modification at 11th nucleotide; and 2′-OMe modifications in the remaining nucleotides; and
(ii) 3′-PS modifications at the 1st, 2nd, 19th, and 20th nucleotides from 5′ end of the sense strand.

In some embodiments, the sense strand having 21 nucleotides in length includes:
(i) 2′-MOE modifications at the 1st, 2nd, 20th and 21st nucleotides from the 5′ end of the sense strand; 2′-F modifications at 7th, 9, and 10th nucleotides from the 5′ end of the sense strand; 2′-deoxy modification at 11th nucleotide; and 2′-OMe modifications in the remaining nucleotides; and
(ii) 3′-PS modifications at the 1st and 2nd nucleotides from 5′ end of the sense strand.

In some embodiments, the sense strand having 21 nucleotides in length includes:
(i) 2′-MOE modifications at the 1st, 2nd, 20th and 21st nucleotides from the 5′ end of the sense strand; 2′-F modifications at 7th, 9, and 10th nucleotides from the 5′ end of the sense strand; 2′-deoxy modification at 11th nucleotide; and 2′-OMe modifications in the remaining nucleotides; and
(ii) 3′-PS modifications at the 1st, 2nd, 19th, and 20th nucleotides from 5′ end of the sense strand.

In certain aspects, a sense strand of the dsRNA as described herein may have a Formula (I′),

(I′)
5′-X₁-X₂-X₃-X₄-X₅-X₆-X₇-X₈-X₉-X₁₀-X₁₁-X₁₂-X₁₃-
X₁₄-X₁₅-X₁₆-X₁₇-X₁₈-X₁₉-X₂₀-X₂₁-3′

wherein:
each X₁, X₂, X₂₀, and X₂₁ is independently a TNA; and
each X₃ to X₁₉ is independently selected from 2′-deoxy modified nucleotide, 2′-MOE modified nucleotide, 2′-F modified nucleotide, and 2′-OMe modified nucleotide.

In some embodiments, the nucleotide sequence of the sense strand of Formula (I′) comprises a sequence selected from the SEQ ID NOs: 761-776 of Table 2. In some embodiments, the nucleotide sequence of the sense strand of Formula (I′) comprises a sequence SEQ ID NO: 761. In some embodiments, the nucleotide sequence of the sense strand of Formula (I′) comprises a sequence SEQ ID NO: 762. In some embodiments, the nucleotide sequence of the sense strand of Formula (I′) comprises a sequence SEQ ID NO: 763. In some embodiments, the nucleotide sequence of the sense strand of Formula (I′) comprises a sequence SEQ ID NO: 764. In some embodiments, the nucleotide sequence of the sense strand of Formula (I′) comprises a sequence SEQ ID NO: 765. In some embodiments, the nucleotide sequence of the sense strand of Formula (I′) comprises a sequence SEQ ID NO: 766. In some embodiments, the nucleotide sequence of the sense strand of Formula (I′) comprises a sequence SEQ ID NO: 767. In some embodiments, the nucleotide sequence of the sense strand of Formula (I′) comprises a sequence SEQ ID NO: 768. In some embodiments, the nucleotide sequence of the sense strand of Formula (I′) comprises a sequence SEQ ID NO: 769. In some embodiments, the nucleotide sequence of the sense strand of Formula (I′) comprises a sequence SEQ ID NO: 770. In some embodiments, the nucleotide sequence of the sense strand of Formula (I′) comprises a sequence SEQ ID NO: 771. In some embodiments, the nucleotide sequence of the sense strand of Formula (I′) comprises a sequence SEQ ID NO: 772. In some embodiments, the nucleotide sequence of the sense strand of Formula (I′) comprises a sequence SEQ ID NO: 773. In some embodiments, the nucleotide sequence of the sense strand of Formula (I′) comprises a sequence SEQ ID NO: 774. In some embodiments, the nucleotide sequence of the sense strand of Formula (I′) comprises a sequence SEQ ID NO: 775. In some embodiments, the nucleotide sequence of the sense strand of Formula (I′) comprises a sequence SEQ ID NO: 776.

In certain aspects, in Formula (I′), X₃ to X₁₉ do not include a 2′-MOE modified nucleotide. In some embodiments, each X₃ to X₁₉ is selected from deoxyribonucleotide, 2′-F modified nucleotides and 2′-OMe modified nucleotides. In some embodiments, at least one of X₃ to X₁₉ is not a deoxyribonucleotide.

In some embodiments, each X_{f}, X_{f+2}, and X_{f+3} is 2′-F modified nucleotide and X_{f+4} is 2′-deoxy modified nucleotide when f is an integer from 3 to 17. In some embodiments, f is 5. In some embodiments, f is 6. In some embodiments, f is 7. In some embodiments, f is 8. In some embodiments, f is 9. In some embodiments, X₅, X₇, and X₈ are 2′-F modified nucleotides, and X₉ is 2′-deoxy modified nucleotide (e.g., dT). In some embodiments, X₆, X₈, and X₉ are 2′-F modified nucleotides and X₁₀ is 2′-deoxy modified nucleotide (e.g., dT). In some embodiments, X₇, X₉, and X₁₀ are 2′-F modified nucleotides, and X₁₁ is 2′-deoxy modified nucleotide (e.g., dT). In some embodiments, X₈, X₁₀, and X₁₁ are 2′-F modified nucleotides, and X₁₂ is 2′-deoxy modified nucleotide (e.g., dT). In some embodiments, X₉, X₁₁, and X₁₂ are 2′-F modified nucleotides, and X₁₃ is 2′-deoxy modified nucleotide (e.g., dT).

In some embodiments, the sense strand includes 2′-OMe modified nucleotides in the remaining positions in the sense strand.

In some embodiments, at least two nucleotides from X₁, X₂, X₁₉, and X₂₀ contain a 3′-PS group, respectively. In some embodiments, two from X₁, X₂, X₁₉, and X₂₀ contains 3′-PS group. In some embodiments, three nucleotides from X₁, X₂, X₁₉, and X₂₀ contain a 3′-PS group, respectively. In some embodiments, each X₁, X₂, X₁₉, and X₂₀ contains a 3′-PS group. In some embodiments, each X₁ and X₂ contains a 3′-PS group.

In some embodiments, at least four from X₁, X₂, X₃, X₄, X₁₇, X₁₈, X₁₉, and/or X₂₀ contain 3′-PS groups. In some embodiments, four from X₁, X₂, X₃, X₄, X₁₇, X₁₈, X₁₉, and/or X₂₀ contain a 3′-PS group, respectively. In some embodiments, six from X₁, X₂, X₃, X₄, X₁₇, X₁₈, X₁₉, and/or X₂₀ contain a 3′-PS group, respectively. In some embodiments, X₁, X₂, X₃, X₄, X₁₇, X₁₈, X₁₉, and X₂₀ contain a 3′-PS group, respectively.

In some embodiments, in X₃ to X₁₈, two to six nucleotides contain 3′-PS groups. In some embodiments, in X₃ to X₁₈, two nucleotides contain a 3′-PS group, respectively. In some embodiments, in X₃ to X₁₈, three nucleotides contain a 3′-PS group, respectively. In some embodiments, in X₃ to X₁₈, four nucleotides contain a 3′-PS group, respectively. In some embodiments, in X₃ to X₁₈, five nucleotides contain a 3′-PS group, respectively. In some embodiments, in X₃ to X₁₈, six nucleotides contain a 3′-PS group, respectively.

In certain aspects, the sense strand includes TNAs positioned at the 1st, 2nd, 20th, and 21st nucleotides from the 5′ end of the sense strand.

In some embodiments, the sense strand having 21 nucleotides in length includes:
(i) TNAs at the 1st, 2nd, 20th and/or 21st nucleotides from the 5′ end of the sense strand; and
(ii) 3′-PS modifications at the 1st, 2nd, 19th, and/or 20th nucleotides from 5′ end of the sense strand.

In some embodiments, the sense strand having 21 nucleotides in length includes:
(i) TNAs at the 1st, 2nd, 20th and/or 21st nucleotides from the 5′ end of the sense strand; 2′-F modifications at 7th, 9th, and 10th nucleotides from the 5′ end of the sense strand; 2′-deoxy modification at 11th nucleotide; and 2′-OMe modifications in the remaining nucleotides.

In some embodiments, the sense strand having 21 nucleotides in length includes:
(i) TNAs at the 1st, 2nd, 20th and/or 21st nucleotides from the 5′ end of the sense strand; 2′-F modifications at 7th, 9th, and 10th nucleotides from the 5′ end of the sense strand; and 2′-deoxy modification at 11th nucleotide; and
(ii) 3′-PS modifications at the 1st and 2nd nucleotides from 5′ end of the sense strand.

In some embodiments, the sense strand having 21 nucleotides in length includes:
(i) TNAs at the 1st, 2nd, 20th and/or 21st nucleotides from the 5′ end of the sense strand; 2′-F modifications at 7th, 9th, and 10th nucleotides from the 5′ end of the sense strand; and 2′-deoxy modification at 11th nucleotide; and
(ii) 3′-PS modifications at the 1st, 2nd, 19th, and 20th nucleotides from 5′ end of the sense strand.

In some embodiments, the sense strand having 21 nucleotides in length includes:
(i) TNAs at the 1st, 2nd, 20th and/or 21st nucleotides from the 5′ end of the sense strand; 2′-F modifications at 7th, 9th, and 10th nucleotides from the 5′ end of the sense strand; 2′-deoxy modification at 11th nucleotide; and 2′-OMe modifications in the remaining nucleotides; and
(ii) 3′-PS modifications at the 1st and 2nd nucleotides from 5′ end of the sense strand.

In some embodiments, the sense strand having 21 nucleotides in length includes:
(i) TNAs at the 1st, 2nd, 20th and/or 21st nucleotides from the 5′ end of the sense strand; 2′-F modifications at 7th, 9th, and 10th nucleotides from the 5′ end of the sense strand; 2′-deoxy modification at 11th nucleotide; and 2′-OMe modifications in the remaining nucleotides; and
(ii) 3′-PS modifications at the 1st, 2nd, 19th, and 20th nucleotides from 5′ end of the sense strand.

In some embodiments, the sense strand having 21 nucleotides in length includes:
(i) TNAs at the 1st, 2nd, 20th and 21st nucleotides from the 5′ end of the sense strand; 2′-F modifications at 7th, 9th, and 10th nucleotides from the 5′ end of the sense strand; 2′-deoxy modification at 11th nucleotide; and 2′-OMe modifications in the remaining nucleotides; and
(ii) 3′-PS modifications at the 1st and 2nd nucleotides from 5′ end of the sense strand.

In some embodiments, the sense strand having 21 nucleotides in length includes:
(i) TNAs at the 1st, 2nd, 20th and 21st nucleotides from the 5′ end of the sense strand; 2′-F modifications at 7th, 9th, and 10th nucleotides from the 5′ end of the sense strand; 2′-deoxy modification at 11th nucleotide; and 2′-OMe modifications in the remaining nucleotides; and
(ii) 3′-PS modifications at the 1st, 2nd, 19th, and 20th nucleotides from 5′ end of the sense strand.

Exemplary modification patterns of sense strands are shown in Table 3.

TABLE 3

| | 2′-MOE | | 2′F | 2′-deoxy | 2′-OMe | |
|---|---|---|---|---|---|---|
| 21-mer SS | modified | | modified | modified | modified | |
| modification | nucleotide | TNA | nucleotide | nucleotide | nucleotide | 3′-PS |
| pattern No. | position | position | position | position | position | linkage |
| SS1 | | | 7, 9, 10, 11 | | 1, 2, 3, 4, | 1, 2 |
| | | | | | 5, 6, 8, 12, | |
| | | | | | 13, 14, 15, | |
| | | | | | 16, 17, 18, | |
| | | | | | 19, 20, 21 | |
| SS2 | 1, 2, 20, 21 | | 7, 9, 10, 11 | | 3, 4, 5, 6, | 1, 2 |
| | | | | | 8, 12, 13, | |
| | | | | | 14, 15, 16, | |
| | | | | | 17, 18, 19 | |
| SS3 | 1, 2, 20, 21 | | 7, 9, 10 | 11 | 3, 4, 5, 6, | 1, 2 |
| | | | | | 8, 12, 13, | |
| | | | | | 14, 15, 16, | |
| | | | | | 17, 18, 19 | |
| SS4 | 1, 2, 20, 21 | | 7, 9, 10, 11 | | 3, 4, 5, 6, | 1, 2, 19, 20 |
| | | | | | 8, 12, 13, | |
| | | | | | 14, 15, 16, | |
| | | | | | 17, 18, 19 | |
| SS5 | 1, 2, 20, 21 | | 7, 9, 10 | 11 | 3, 4, 5, 6, | 1, 2, 19, 20 |
| | | | | | 8, 12, 13, | |
| | | | | | 14, 15, 16, | |
| | | | | | 17, 18, 19 | |
| SS6 | | 1, 2, 20, | 7, 9, 10, 11 | | 3, 4, 5, 6, | 1, 2 |
| | | 21 | | | 8, 12, 13, | |
| | | | | | 14, 15, 16, | |
| | | | | | 17, 18, 19 | |
| SS7 | | 1, 2, 20, | 7, 9, 10 | 11 | 3, 4, 5, 6, | 1, 2 |
| | | 21 | | | 8, 12, 13, | |
| | | | | | 14, 15, 16, | |
| | | | | | 17, 18, 19 | |
| SS8 | | 1, 2, 20, | 7, 9, 10, 11 | | 3, 4, 5, 6, | 1, 2, 19, 20 |
| | | 21 | | | 8, 12, 13, | |
| | | | | | 14, 15, 16, | |
| | | | | | 17, 18, 19 | |
| SS9 | | 1, 2, 20, | 7, 9, 10 | 11 | 3, 4, 5, 6, | 1, 2, 19, 20 |
| | | 21 | | | 8, 12, 13, | |
| | | | | | 14, 15, 16, | |
| | | | | | 17, 18, 19 | |
| SS10 | 1, 2, 20, 21 | None | At least | At least one | At least | 1, 2, 19, |
| | | | one among | among the | one among | and/or 20 |
| | | | the | remaining | the | |
| | | | remaining | positions | remaining | |
| | | | positions | | positions | |
| SS11 | None | 1, 2, 20, | At least | At least one | At least | 1, 2, 19, |
| | | 21 | one among | among the | one among | and/or 20 |
| | | | the | remaining | the | |
| | | | remaining | positions | remaining | |
| | | | positions | | positions | |

In some embodiments, the sense strand having 21 nucleotides in length has the modification pattern of SS1. In some embodiments, the sense strand having 21 nucleotides in length has the modification pattern of SS2. In some embodiments, the sense strand having 21 nucleotides in length has the modification pattern of SS3. In some embodiments, the sense strand having 21 nucleotides in length has the modification pattern of SS4. In some embodiments, the sense strand having 21 nucleotides in length has the modification pattern of SS5. In some embodiments, the sense strand having 21 nucleotides in length has the modification pattern of SS6. In some embodiments, the sense strand having 21 nucleotides in length has the modification pattern of SS7. In some embodiments, the sense strand having 21 nucleotides in length has the modification pattern of SS8. In some embodiments, the sense strand having 21 nucleotides in length has the modification pattern of SS9. In some embodiments, the sense strand having 21 nucleotides in length has the modification pattern of SS10. In some embodiments, the sense strand having 21 nucleotides in length has the modification pattern of SS11. In some embodiments, the sense strand has a modification pattern of SS1, SS2, SS3, SS4, SS5, SS6, SS7, SS8, SS9, SS10, or SS11, and comprises a nucleotide sequence selected from SEQ ID NOs: 761-776. In some embodiments, the sense strand has a modification pattern of SS1, SS2, SS3, SS4, SS5, SS6, SS7, SS8, SS9, SS10, or SS11, and comprises SEQ ID NO: 761. In some embodiments, the sense strand has a modification pattern of SS1, SS2, SS3, SS4, SS5, SS6, SS7, SS8, SS9, SS10, or SS11, and comprises SEQ ID NO: 762. In some embodiments, the sense strand has a modification pattern of SS1, SS2, SS3, SS4, SS5, SS6, SS7, SS8, SS9, SS10, or SS11, and comprises SEQ ID NO: 763. In some embodiments, the sense strand has a modification pattern of SS1, SS2, SS3, SS4, SS5, SS6, SS7, SS8, SS9, SS10, or SS11, and comprises SEQ ID NO: 764. In some embodiments, the sense strand has a modification pattern of SS1, SS2, SS3, SS4, SS5, SS6, SS7, SS8, SS9, SS10, or SS11, and comprises SEQ ID NO: 765. In some embodiments, the sense strand has a modification pattern of SS1, SS2, SS3, SS4, SS5, SS6, SS7, SS8, SS9, SS10, or SS11, and comprises SEQ ID NO: 766. In some embodiments, the nucleotide sequence of the sense strand comprises SEQ ID NO: 767. In some embodiments, the sense strand has a modification pattern of SS1, SS2, SS3, SS4, SS5, SS6, SS7, SS8, SS9, SS10, or SS11, and comprises SEQ ID NO: 768. In some embodiments, the sense strand has a modification pattern of SS1, SS2, SS3, SS4, SS5, SS6, SS7, SS8, SS9, SS10, or SS11, and comprises SEQ ID NO: 769. In some embodiments, the sense strand has a modification pattern of SS1, SS2, SS3, SS4, SS5, SS6, SS7, SS8, SS9, SS10, or SS11, and comprises SEQ ID NO: 770. In some embodiments, the sense strand has a modification pattern of SS1, SS2, SS3, SS4, SS5, SS6, SS7, SS8, SS9, SS10, or SS11, and comprises SEQ ID NO: 771. In some embodiments, the sense strand has a modification pattern of SS1, SS2, SS3, SS4, SS5, SS6, SS7, SS8, SS9, SS10, or SS11, and comprises SEQ ID NO: 772. In some embodiments, the sense strand has a modification pattern of SS1, SS2, SS3, SS4, SS5, SS6, SS7, SS8, SS9, SS10, or SS11, and comprises SEQ ID NO: 773. In some embodiments, the sense strand has a modification pattern of SS1, SS2, SS3, SS4, SS5, SS6, SS7, SS8, SS9, SS10, or SS11, and comprises SEQ ID NO: 774. In some embodiments, the sense strand has a modification pattern of SS1, SS2, SS3, SS4, SS5, SS6, SS7, SS8, SS9, SS10, or SS11, and comprises SEQ ID NO: 775. In some embodiments, the sense strand has a modification pattern of SS1, SS2, SS3, SS4, SS5, SS6, SS7, SS8, SS9, SS10, or SS11, and comprises SEQ ID NO: 776. In some embodiments, the sense strand has a modification pattern of SS1 and comprises SEQ ID No: 761. In some embodiments, the sense strand has a modification pattern of SS2 and comprises SEQ ID No: 761. In some embodiments, the sense strand has a modification pattern of SS3 and comprises SEQ ID No: 761. In some embodiments, the sense strand has a modification pattern of SS4 and comprises SEQ ID No: 761. In some embodiments, the sense strand has a modification pattern of SS5 and comprises SEQ ID No: 761. In some embodiments, the sense strand has a modification pattern of SS6 and comprises SEQ ID No: 761. In some embodiments, the sense strand has a modification pattern of SS7 and comprises SEQ ID No: 761. In some embodiments, the sense strand has a modification pattern of SS8 and comprises SEQ ID No: 761. In some embodiments, the sense strand has a modification pattern of SS9 and comprises SEQ ID No: 761. In some embodiments, the sense strand has a modification pattern of SS10 and comprises SEQ ID No: 761. In some embodiments, the sense strand has a modification pattern of SS11 and comprises SEQ ID No: 761.

### Antisense Strand (AS)

In certain aspects, an antisense strand of the dsRNA as described herein are substantially (e.g., greater than about 80%, 85%, 90%, or 95% of the total nucleotides) made of modified nucleotides. In another certain aspect, the antisense strand is entirely made of modified nucleotides.

In certain aspects, the first nucleotide from the 5′ end of the antisense strand may contain an additional phosphate group or a variant thereof (e.g., phosphorothioate, phosphorodithioate, methylphosphonate, methylene phosphonate, or vinyl phosphonate (VP)) attached or linked to the 5′ terminal group of the first nucleotide) attached or linked to the 5′ terminal group of the first nucleotide.

In certain aspects, the antisense strand includes 5′-vinyl phosphonate (5′-VP) group at the first nucleotide from the 5′ end in the antisense strand. The “5′-VP” is a chemical moiety having the structure of

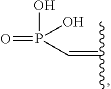 [SMILES: *=CP(=O)(O)O]

or a pharmaceutically acceptable salt thereof, where the wavy line represent the point of attachment to the 5′ carbon of the pentofuranosyl sugar of a nucleotide.

In some embodiments, the first nucleotide from the 5′ end in the antisense strand includes (E)-vinyl phosphonate (VP) having a structure of

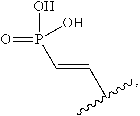 [SMILES: */C=C/P(=O)(O)O]

or a pharmaceutically acceptable salt thereof, wherein the wavy line presents the point of attachment to the 4′ carbon of the pentofuranosyl sugar of a nucleotide. In some embodiments, the first nucleotide from the 5′ end in the antisense strand includes (Z)-vinyl phosphonate having a structure of

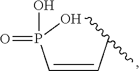 [SMILES: */C=C\P(=O)(O)O]

or a pharmaceutically acceptable salt thereof, wherein the wavy line presents the point of attachment to the 4′ carbon of the pentofuranosyl sugar of a nucleotide.

In some embodiments, the first nucleotide from the 5′ end of the antisense strand has a structure of

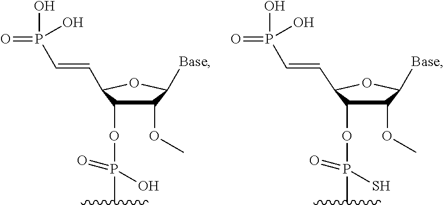

or a pharmaceutically acceptable salt thereof. In some embodiments, the first nucleotide from the 5′ end of the antisense strand has a structure of

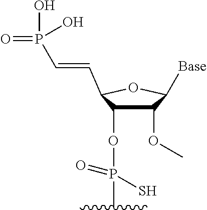

or a pharmaceutically acceptable salt thereof. In some embodiments, the first nucleotide from the 5′ end of the antisense strand has a structure of

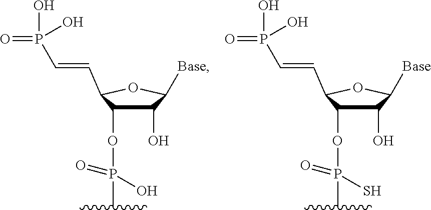

or a pharmaceutically acceptable salt thereof. In some embodiments, the first nucleotide from the 5′ end of the antisense strand has a structure of

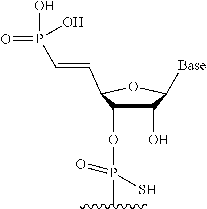

or a pharmaceutically acceptable salt thereof.

In some embodiments, the first nucleotide from the 5′ end of the antisense strand has a structure of

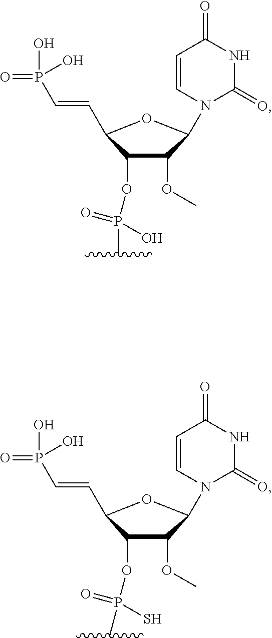

or a pharmaceutically acceptable salt thereof, wherein [symbol] is an attachment point to the adjacent nucleotides. In some embodiments, the first nucleotide from the 5′ end of the antisense strand has a structure of

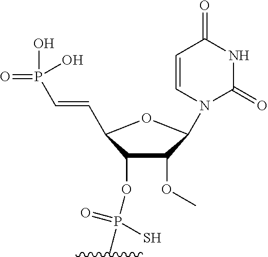

or a pharmaceutically acceptable salt thereof. In some embodiments, the first nucleotide from the 5′ end of the antisense strand has a structure of

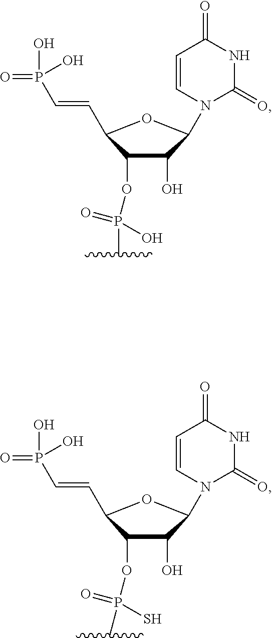

or a pharmaceutically acceptable salt thereof, wherein [symbol] is an attachment point to the adjacent nucleotides. In some embodiments, the first nucleotide from the 5′ end of the antisense strand has a structure of

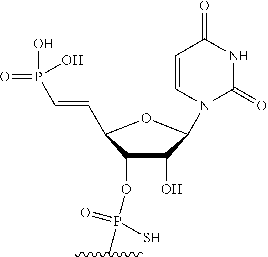

or a pharmaceutically acceptable salt thereof.

In certain aspects, the antisense strand of the dsRNA as described herein includes two or more 2′-F modifications. In some embodiments, the antisense strand of the dsRNA includes two, three, four, five, six, seven, or eight 2′-F modified nucleotides. In some embodiments, the antisense strand includes two 2′-F modified nucleotides. In some embodiments, the antisense strand includes three 2′-F modified nucleotides. In some embodiments, the antisense strand includes four 2′-F modified nucleotides. In some embodiments, the antisense strand includes five 2′-F modified nucleotides. In some embodiments, the antisense strand includes six 2′-F modified nucleotides. In some embodiments, the antisense strand includes seven 2′-F modified nucleotides. In some embodiments, the antisense strand includes eight 2′-F modified nucleotides. In some embodiments, two contiguous 2′-F modified nucleotides locate in the antisense strand. In some embodiments, three contiguous 2′-F modified nucleotides locate in the antisense strand. In some embodiments, four contiguous 2′-F modified nucleotides locate in the antisense strand.

In certain aspects, the antisense strand is 23 nucleotides in length. In some embodiments, the antisense includes comprises two, three, or four 2′-F modifications positioned at the 2nd, 6th, 14th, and/or 16th nucleotide from 5′ end of the antisense strand. In some embodiments, the antisense includes two 2′-F modifications positioned at the 2nd, 6th, 14th, and/or 16th nucleotide from 5′ end of the antisense strand. In some embodiments, the antisense includes three 2′-F modifications positioned at the 2nd, 6th, 14th, and/or 16th nucleotide from 5′ end of the antisense strand. In some embodiments, the antisense includes 2′-F modifications positioned at the 2nd, 6th, 14th, and 16th nucleotide from 5′ end of the antisense strand.

In certain aspects, an antisense strand of the dsRNA as described herein does not include a 2′-MOE modification. Alternatively, in certain aspects, the antisense strand includes one to four 2′-MOE modified nucleotides. In some embodiments, the antisense strand includes one 2′-MOE modified nucleotide. In some embodiments, the antisense strand includes two 2′-MOE modified nucleotides. In some embodiments, the antisense strand includes three 2′-MOE modified nucleotides. In some embodiments, the antisense strand includes four 2′-MOE modified nucleotides.

In certain aspects, the antisense strand includes at least one GNA. In some embodiments, the antisense strand includes only one GNA.

In certain aspects, the antisense strand is 23 nucleotides in length. In some embodiments, the antisense strand includes only one GNA at the 4th nucleotide from 5′ end of the antisense strand. In some embodiments, the antisense strand includes only one GNA at the 5th nucleotide from 5′ end of the antisense strand. In some embodiments, the antisense strand includes only one GNA at the 6th nucleotide from 5′ end of the antisense strand. In some embodiments, the antisense strand includes only one GNA at the 7th nucleotide from 5′ end of the antisense strand.

In certain aspects, the antisense strand includes at least one TNA. In some embodiments, the antisense strand includes only one TNA.

In certain aspects, the antisense strand is 23 nucleotides in length. In some embodiments, the antisense strand includes only one TNA at the 3rd nucleotide from 5′ end of the antisense strand. In some embodiments, the antisense strand includes only one TNA at the 4th nucleotide from 5′ end of the antisense strand. In some embodiments, the antisense strand includes only one TNA at the 5th nucleotide from 5′ end of the antisense strand. In some embodiments, the antisense strand includes only one TNA at the 6th nucleotide from 5′ end of the antisense strand. In some embodiments, the antisense strand includes only one TNA at the 7th nucleotide from 5′ end of the antisense strand. In some embodiments, the antisense strand includes only one TNA at the 8th nucleotide from 5′ end of the antisense strand. In some embodiments, the antisense strand includes one or two TNA at the position 3, 5, 6, and/or 7 from 5′ end of the antisense strand, where the antisense strand has a nucleotide sequence of any one selected from SEQ ID NOs: 777-792. In some embodiments, the antisense strand includes one or two TNA at the position 3, 5, 6, and/or 7 from 5′ end of the antisense strand, where the antisense strand has a nucleotide sequence of SEQ ID NO:777. In some embodiments, the antisense strand includes one TNA at the position 3, 5, 6, or 7 from 5′ end of the antisense strand, where the antisense strand has a nucleotide sequence of any one selected from SEQ ID NOs: 777-792. In some embodiments, the antisense strand includes one TNA at the position 3, 5, 6, or 7 from 5′ end of the antisense strand, where the antisense strand has a nucleotide sequence of SEQ ID NO: 777. In some embodiments, the antisense strand includes two TNA at the position 3, and 5, 6, or 7 from 5′ end of the antisense strand, where the antisense strand has a nucleotide sequence of any one selected from SEQ ID NOs: 777-792. In some embodiments, the antisense strand includes two TNA at the position 3, and 5, 6, or 7 from 5′ end of the antisense strand, where the antisense strand has a nucleotide sequence of SEQ ID NO: 777.

In certain aspects, the antisense strand includes at least one 2′-deoxy modified nucleotide (e.g., dT, dA, dG, or dC). In some embodiments, the antisense strand includes only one 2′-deoxy modified nucleotide (e.g., dT, dA, dG, or dC).

In certain aspects, the antisense strand is 23 nucleotides in length. In some embodiments, the antisense strand includes only one 2′-deoxy modified nucleotide (e.g., dT, dA, dG, or dC) at the 3rd nucleotide from 5′ end of the antisense strand. In some embodiments, the antisense strand includes only one 2′-deoxy modified nucleotide (e.g., dT, dA, dG, or dC) at the 4th nucleotide from 5′ end of the antisense strand. In some embodiments, the antisense strand includes only one 2′-deoxy modified nucleotide (e.g., dT, dA, dG, or dC) at the 5th nucleotide from 5′ end of the antisense strand. In some embodiments, the antisense strand includes only one 2′-deoxy modified nucleotide (e.g., dT, dA, dG, or dC) at the 6th nucleotide from 5′ end of the antisense strand. In some embodiments, the antisense strand includes only one 2′-deoxy modified nucleotide (e.g., dT, dA, dG, or dC) at the 7th nucleotide from 5′ end of the antisense strand. In some embodiments, the antisense strand includes only one 2′-deoxy modified nucleotide (e.g., dT, dA, dG, or dC) at the 8th nucleotide from 5′ end of the antisense strand.

In certain aspects, the antisense strand includes two, three, or four phosphorothioate (PS) linkages between nucleosides. In certain aspects, the antisense strand is 23 nucleotides in length. In some embodiments, the antisense strand includes two 3′-PS modifications positioned at the 1st, 2nd, 21st, and/or 22nd nucleotides from 5′ end of the antisense strand. In some embodiments, the antisense strand includes three 3′-PS modifications positioned at the 1st, 2nd, 21st, and/or 22nd nucleotides from 5′ end of the antisense strand. In some embodiments, the antisense strand includes 3′-PS modifications positioned at the 1st, 2nd, 21st, and 22nd nucleotides from 5′ end of the antisense strand.

In certain aspects, the antisense strand includes two to eight phosphorothioate (PS) linkages between nucleosides.

In certain aspects, the antisense strand is 23 nucleotides in length. In some embodiments, the antisense strand includes two 3′-PS modified nucleotides positioned at the 1st, 2nd, 3rd, 4th, 19th, 20th, 21st, and/or 22nd nucleotides from 5′ end of the antisense strand. In some embodiments, the antisense strand includes four 3′-PS modified nucleotides positioned at the 1st, 2nd, 3rd, 4th, 19th, 20th, 21st, and/or 22nd nucleotides from 5′ end of the antisense strand. In some embodiments, the antisense strand includes six 3′-PS modified nucleotides positioned at the 1st, 2nd, 3rd, 4th, 19th, 20th, 21st, and/or 22nd nucleotides from 5′ end of the antisense strand. In some embodiments, the antisense strand includes 3′-PS modified nucleotides positioned at the 1st, 2nd, 3rd, 4th, 19th, 20th, 21st, and 22nd nucleotides from 5′ end of the antisense strand.

In certain aspects, the antisense strand is 23 nucleotides in length. In some embodiments, at least one of the PS groups at the 1st, 2nd, 3rd, 4th, 19th, 20th, 21st, and/or 22nd nucleotides from 5′ end of the antisense strand is a stereopure Rp isomer. In some embodiments, at least one of the PS groups at the 1st, 2nd, 21st, and/or 22nd nucleotides from 5′ end of the antisense strand is a stereopure Rp isomer. In some embodiments, at least one of the PS groups at the 1st and/or 22nd nucleotides from 5′ end of the antisense strand is a stereopure Rp isomer. In some embodiments, the PS group at the 1st nucleotide from 5′ end of the antisense strand is a stereopure Rp isomer. In some embodiments, the PS group at the 22nd nucleotide from 5′ end of the antisense strand is a stereopure Rp isomer. In some embodiments, the PS groups at the 1st and 22nd nucleotides from 5′ end of the antisense strand are stereopure Rp isomers.

In certain aspects, the antisense strand is 23 nucleotides in length. In some embodiments, at least one of the PS groups at the 1st, 2nd, 3rd, 4th, 19th, 20th, 21st, and/or 22nd nucleotides from 5′ end of the antisense strand is a stereopure Sp isomer. In some embodiments, at least one of the PS groups at the 1st, 2nd, 21st, and/or 22nd nucleotides from 5′ end of the antisense strand is a stereopure Sp isomer. In some embodiments, at least one of the PS groups at the 1st and/or 22nd nucleotides from 5′ end of the antisense strand is a stereopure Sp isomer. In some embodiments, the PS group at the 1st nucleotide from 5′ end of the antisense strand is a stereopure Sp isomer. In some embodiments, the PS group at the 22nd nucleotide from 5′ end of the antisense strand is a stereopure Sp isomer. In some embodiments, the PS groups at the 1st and 22nd nucleotides from 5′ end of the antisense strand are stereopure Sp isomers.

In certain aspects, the antisense strand is 23 nucleotides in length. In some embodiments, at least one of the PS groups at the 1st, 2nd, 3rd, 4th, 19th, 20th, 21st, and/or 22nd nucleotides from 5′ end of the antisense strand is stereopure Sp isomer

In certain aspects, an antisense strand of dsRNA may have a Formula (II):

(II)
5′-X_{1′}-X_{2′}-X_{3′}-X_{4′}-X_{5′}-X_{6′}-X_{7′}-X_{8′}-X_{9′}-X_{10′}-X_{11′}-
X_{12′}-X_{13′}-X_{14′}-X_{15′}-X_{16′}-X_{17′}-X_{18′}-X_{19′}-X_{20′}-X_{21′}-
X_{22′}-X_{23′}-3′

wherein:
each X_{1′} to X_{23′} is independently selected from a 2′-deoxy modified nucleotide, 2′-F modified nucleotide, 2′-OMe modified nucleotide, 2′-MOE modified nucleotide, TNA, and GNA; and
X_{1′} further includes a 5′-(E)-vinyl phosphonate group.

In some embodiments, the nucleotide sequence of the antisense strand of Formula (II) is selected from SEQ ID NOs: 777-792. In some embodiments, the nucleotide sequence of the antisense strand of Formula (II) comprises SEQ ID NO: 777. In some embodiments, the nucleotide sequence of the antisense strand of Formula (II) comprises SEQ ID NO: 778. In some embodiments, the nucleotide sequence of the antisense strand of Formula (II) comprises SEQ ID NO: 779. In some embodiments, the nucleotide sequence of the antisense strand of Formula (II) comprises SEQ ID NO: 780. In some embodiments, the nucleotide sequence of the antisense strand of Formula (II) comprises SEQ ID NO: 781. In some embodiments, the nucleotide sequence of the antisense strand of Formula (II) comprises SEQ ID NO: 782. In some embodiments, the nucleotide sequence of the antisense strand of Formula (II) comprises SEQ ID NO: 783. In some embodiments, the nucleotide sequence of the antisense strand of Formula (II) comprises SEQ ID NO: 784. In some embodiments, the nucleotide sequence of the antisense strand of Formula (II) comprises SEQ ID NO: 785. In some embodiments, the nucleotide sequence of the antisense strand of Formula (II) comprises SEQ ID NO: 786. In some embodiments, the nucleotide sequence of the antisense strand of Formula (II) comprises SEQ ID NO: 787. In some embodiments, the nucleotide sequence of the antisense strand of Formula (II) comprises SEQ ID NO: 788. In some embodiments, the nucleotide sequence of the antisense strand of Formula (II) comprises SEQ ID NO: 789. In some embodiments, the nucleotide sequence of the antisense strand of Formula (II) comprises SEQ ID NO: 790. In some embodiments, the nucleotide sequence of the antisense strand of Formula (II) comprises SEQ ID NO: 791. In some embodiments, the nucleotide sequence of the antisense strand of Formula (II) comprises SEQ ID NO: 792.

In certain aspects, in Formula (II), X_{1′} to X_{23′} do not include a 2′-MOE modified nucleotide. In some embodiments, each X_{1′} to X_{23′} is independently selected from 2′-F modified nucleotides and 2′-OMe modified nucleotides.

Alternatively, in certain aspects, in Formula (II), X_{1′} to X_{23′} include one to four 2′-MOE modified nucleotides. In some embodiments, one of X_{1′}, X_{9′}, X_{10′}, and X_{23′} may be 2′-MOE modified nucleotide. In some embodiments, two of X_{1′}, X_{9′}, X_{10′}, and X_{23′} may be 2′-MOE modified nucleotides. In some embodiments, three of X_{1′}, X_{9′}, X_{10′}, and X_{23′} may be 2′-MOE modified nucleotides. In some embodiments, X_{1′}, X_{9′}, X_{10′}, and X_{23′} may be 2′-MOE modified nucleotide.

In some embodiments, X_{2′} is a 2′-F modified nucleotide. In some embodiments, X_{6′} is a 2′-F modified nucleotide. In some embodiments, X_{14′} is a 2′-F modified nucleotide. In some embodiments, X_{16′} is a 2′-F modified nucleotide. In some embodiments, two of X_{2′}, X_{6′}, X_{14′} and X₁₆, are 2′-F modified nucleotides. In some embodiments, three of X_{2′}, X_{6′}, X_{14′} and X_{16′} are 2′-F modified nucleotides. In some embodiments, each X_{2′}, X_{6′}, X_{14′} and X_{16′} is a 2′-F modified nucleotide.

In some embodiments, X_{1′} to X_{23′} may include at least one GNA. In some embodiments, X_{1′} to X_{23′} may include only one GNA. In some embodiments, X_{3′} is a GNA. In some embodiments, X_{4′} is a GNA. In some embodiments, X_{5′} is a GNA. In some embodiments, X_{6′} is a GNA. In some embodiments, X_{7′} is a GNA.

In some embodiments, X_{1′} to X_{23′} may include at least one TNA. In some embodiments, X_{1′} to X_{23′} may include only one TNA. In some embodiments, X_{3′} is a TNA. In some embodiments, X_{4′} is a TNA. In some embodiments, X_{5′} is a TNA. In some embodiments, X_{6′} is a TNA. In some embodiments, X_{7′} is a TNA.

In some embodiments, X_{1′} to X_{23′} may include at least one 2′-deoxy modified nucleotide (e.g., dT, dA, dG, or dC). In some embodiments, X_{1′} to X_{23′} may include only one 2′-deoxy modified nucleotide (e.g., dT, dA, dG, or dC). In some embodiments, X_{3′} is a 2′-deoxy modified nucleotide (e.g., dT, dA, dG, or dC). In some embodiments, X_{4′} is a 2′-deoxy modified nucleotide (e.g., dT, dA, dG, or dC). In some embodiments, X_{5′} is a 2′-deoxy modified nucleotide (e.g., dT, dA, dG, or dC). In some embodiments, X_{6′} is a 2′-deoxy modified nucleotide (e.g., dT, dA, dG, or dC). In some embodiments, X_{7′} is a 2′-deoxy modified nucleotide (e.g., dT, dA, dG, or dC). In some embodiments, X_{1′} to X_{23′} may include only dT. In some embodiments, X_{3′} is dT. In some embodiments, X_{4′} is dT. In some embodiments, X_{5′} is dT. In some embodiments, X_{6′} is dT. In some embodiments, X_{7′} is dT.

In some embodiments, the antisense strand includes 2′-OMe modified nucleotides in the remaining positions in the antisense strand.

In some embodiments, at least two from X_{1′}, X_{2′}, X_{21′}, and X_{22′} contain 3′-PS groups. In some embodiments, two from X_{1′}, X_{2′}, X_{21′}, and X_{22′} contain a 3′-PS group, respectively. In some embodiments, three from X_{1′}, X_{2′}, X_{21′}, and X_{22′} contain a 3′-PS group. In some embodiments, each X_{1′}, X_{2′}, X_{21′}, and X_{22′} contains a 3′-PS group.

In some embodiments, at least four from X_{1′}, X_{2′}, X_{3′}, X_{4′}, X_{19′}, X_{20′}, X_{21′}, and X_{22′} contain 3′-PS groups. In some embodiments, four from X_{1′}, X_{2′}, X_{3′}, X_{4′}, X_{19′}, X_{20′}, X_{21′}, and X_{22′} contain a 3′-PS group, respectively. In some embodiments, six from X_{1′}, X_{2′}, X_{3′}, X_{4′}, X_{19′}, X_{20′}, X_{21′}, and X_{22′} contain a 3′-PS group, respectively. In some embodiments, X_{1′}, X_{2′}, X_{3′}, X_{4′}, X_{19′}, X_{20′}, X_{21′}, and X_{22′} contain a 3′-PS group, respectively.

In some embodiments, in X_{3′} to X_{20′}, two to six nucleotides contain 3′-PS groups. In some embodiments, in X_{3′} to X_{20′}, two nucleotides contain a 3′-PS group, respectively. In some embodiments, in X_{3′} to X_{20′}, three nucleotides contain a 3′-PS group, respectively. In some embodiments, in X_{3′} to X_{20′}, four nucleotides contain a 3′-PS group, respectively. In some embodiments, in X_{3′} to X_{20′}, five nucleotides contain a 3′-PS group, respectively. In some embodiments, in X_{3′} to X_{20′}, six nucleotides contain a 3′-PS group, respectively.

In certain aspects, the antisense strand includes 5′-(E)-VP modified nucleotide at the first nucleotide from 5′ end of the antisense strand. In some embodiments, the antisense strand includes a 5′-(E)-VP-2′-OMe modified nucleotide at the first nucleotide from 5′ end of the antisense strand.

In some embodiments, the antisense strand having 23 nucleotides in length includes:
(i) a 5′-(E)-VP-2′-OMe modification at the first nucleotide from 5′ end of the antisense strand; and
(ii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; GNA at 5th nucleotide from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 14th, and/or 16th nucleotides from the 5′ end of the antisense strand; GNA at 6th nucleotide from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; GNA at 7th nucleotide from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; TNA at 3rd nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; TNA at 5th nucleotide from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 14th, and/or 16th nucleotides from the 5′ end of the antisense strand; TNA at 6^{th} nucleotide from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; TNA at 7th nucleotide from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; a 2′-deoxy modification at 5th nucleotide from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 14th, and/or 16th nucleotides from the 5′ end of the antisense strand; a 2′-deoxy modification at 6^{th} nucleotide from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; a 2′-deoxy modification at 7th nucleotide from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; GNA at 3rd and 5th nucleotides from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 14th, and/or 16th nucleotides from the 5′ end of the antisense strand; GNA at 3rd and 6th nucleotides from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; GNA at 3rd and 7th nucleotides from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; TNA at 3rd and 5th nucleotides from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 14th, and/or 16th nucleotides from the 5′ end of the antisense strand; TNA at 3rd and 6th nucleotides from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; TNA at 3rd and 7th nucleotides from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; a 2′-deoxy modification at 3rd and 5th nucleotides from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 14th, and/or 16th nucleotides from the 5′ end of the antisense strand; a 2′-deoxy modification at 3rd and 6th nucleotide from the 5′ end of the antisense strand; and 2′-OMe modifications in the remaining nucleotides; and
(iii) 3′-PS modifications at the 1st, 2nd, 21st, and/or 22nd nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2nd, 6th, 14th, and/or 16th nucleotides from the 5′ end of the antisense strand; a 2′-deoxy modification at 3rd and 7th nucleotides from the 5′ end of the antisense strand; and 2′-OMe modifications in the remaining nucleotides; and
(iii) 3′-PS modifications at the 1 st, 2nd, 21 st, and/or 22nd nucleotides from 5′ end of the antisense strand.

Exemplary modification patterns of antisense strands are shown in Table 4.

TABLE 4

| | | | | | 2′- | | |
|---|---|---|---|---|---|---|---|
| | 5′-VP | | | | deoxy | 2′-OMe | |
| 23-mer AS | modified | 2′-F | | | modified | modified | |
| modification | nucleotide | modified | GNA | TNA | nucleotide | nucleotide | 3′-PS |
| pattern | position | nucleotide | position | position | position | position | linkage |
| AS1 | — | 2, 6, 14, 16 | | | | 1, 3, 4, 5, 7, | 1, 2, 21, |
| | | | | | | 8, 9, 10, 11, | 22 |
| | | | | | | 12, 13, 15, | |
| | | | | | | 17, 18, 19, | |
| | | | | | | 20, 21, 22, | |
| | | | | | | 23 | |
| AS2 | 1 | 2, 6, 14, 16 | | | | 1, 3, 4, 5, 7, | 1, 2, 21, |
| | | | | | | 8, 9, 10, 11, | 22 |
| | | | | | | 12, 13, 15, | |
| | | | | | | 17, 18, 19, | |
| | | | | | | 20, 21, 22, | |
| | | | | | | 23 | |
| AS3 | 1 | 2, 6, 14, 16 | 5 | | | 1, 3, 4, 7, 8, | 1, 2, 21, |
| | | | | | | 9, 10, 11, | 22 |
| | | | | | | 12, 13, 15, | |
| | | | | | | 17, 18, 19, | |
| | | | | | | 20, 21, 22, | |
| | | | | | | 23 | |
| AS4 | 1 | 2, 14, 16 | 6 | | | 1, 3, 4, 5, 7, | 1, 2, 21, |
| | | | | | | 8, 9, 10, 11, | 22 |
| | | | | | | 12, 13, 15, | |
| | | | | | | 17, 18, 19, | |
| | | | | | | 20, 21, 22, | |
| | | | | | | 23 | |
| AS5 | 1 | 2, 6, 14, 16 | 7 | | | 1, 3, 4, 5, 8, | 1, 2, 21, |
| | | | | | | 9, 10, 11, | 22 |
| | | | | | | 12, 13, 15, | |
| | | | | | | 17, 18, 19, | |
| | | | | | | 20, 21, 22, | |
| | | | | | | 23 | |
| AS6 | 1 | 2, 6, 14, 16 | | 3 | | 1, 4, 5, 7, 8, | 1, 2, 21, |
| | | | | | | 9, 10, 11, | 22 |
| | | | | | | 12, 13, 15, | |
| | | | | | | 17, 18, 19, | |
| | | | | | | 20, 21, 22, | |
| | | | | | | 23 | |
| AS7 | 1 | 2, 6, 14, 16 | | 5 | | 1, 3, 4, 7, 8, | 1, 2, 21, |
| | | | | | | 9, 10, 11, | 22 |
| | | | | | | 12, 13, 15, | |
| | | | | | | 17, 18, 19, | |
| | | | | | | 20, 21, 22, | |
| | | | | | | 23 | |
| AS8 | 1 | 2, 14, 16 | | 6 | | 1, 3, 4, 5, 7, | 1, 2, 21, |
| | | | | | | 8, 9, 10, 11, | 22 |
| | | | | | | 12, 13, 15, | |
| | | | | | | 17, 18, 19, | |
| | | | | | | 20, 21, 22, | |
| | | | | | | 23 | |
| AS9 | 1 | 2, 6, 14, 16 | | 7 | | 1, 3, 4, 5, 8, | 1, 2, 21, |
| | | | | | | 9, 10, 11, | 22 |
| | | | | | | 12, 13, 15, | |
| | | | | | | 17, 18, 19, | |
| | | | | | | 20, 21, 22, | |
| | | | | | | 23 | |
| AS10 | 1 | 2, 6, 14, 16 | | | 5 | 1, 3, 4, 7, 8, | 1, 2, 21, |
| | | | | | | 9, 10, 11, | 22 |
| | | | | | | 12, 13, 15, | |
| | | | | | | 17, 18, 19, | |
| | | | | | | 20, 21, 22, | |
| | | | | | | 23 | |
| AS11 | 1 | 2, 14, 16 | | | 6 | 1, 3, 4, 5, 7, | 1, 2, 21, |
| | | | | | | 8, 9, 10, 11, | 22 |
| | | | | | | 12, 13, 15, | |
| | | | | | | 17, 18, 19, | |
| | | | | | | 20, 21, 22, | |
| | | | | | | 23 | |
| AS12 | 1 | 2, 6, 14, 16 | | | 7 | 1, 3, 4, 5, 8, | 1, 2, 21, |
| | | | | | | 9, 10, 11, | 22 |
| | | | | | | 12, 13, 15, | |
| | | | | | | 17, 18, 19, | |
| | | | | | | 20, 21, 22, | |
| | | | | | | 23 | |
| AS13 | 1 | 2, 6, 14, 16 | 3, 5 | | | 1, 4, 7, 8, 9, | 1, 2, 21, |
| | | | | | | 10, 11, 12, | 22 |
| | | | | | | 13, 15, 17, | |
| | | | | | | 18, 19, 20, | |
| | | | | | | 21, 22, 23 | |
| AS14 | 1 | 2, 14, 16 | 3, 6 | | | 1, 4, 5, 7, 8, | 1, 2, 21, |
| | | | | | | 9, 10, 11, | 22 |
| | | | | | | 12, 13, 15, | |
| | | | | | | 17, 18, 19, | |
| | | | | | | 20, 21, 22, | |
| | | | | | | 23 | |
| AS15 | 1 | 2, 6, 14, 16 | 3, 7 | | | 1, 4, 5, 8, 9, | 1, 2, 21, |
| | | | | | | 10, 11, 12, | 22 |
| | | | | | | 13, 15, 17, | |
| | | | | | | 18, 19, 20, | |
| | | | | | | 21, 22, 23 | |
| AS16 | 1 | 2, 6, 14, 16 | | 3, 5 | | 1, 4, 7, 8, 9, | 1, 2, 21, |
| | | | | | | 10, 11, 12, | 22 |
| | | | | | | 13, 15, 17, | |
| | | | | | | 18, 19, 20, | |
| | | | | | | 21, 22, 23 | |
| AS17 | 1 | 2, 14, 16 | | 3, 6 | | 1, 4, 5, 7, 8, | 1, 2, 21, |
| | | | | | | 9, 10, 11, | 22 |
| | | | | | | 12, 13, 15, | |
| | | | | | | 17, 18, 19, | |
| | | | | | | 20, 21, 22, | |
| | | | | | | 23 | |
| AS18 | 1 | 2, 6, 14, 16 | | 3, 7 | | 1, 4, 5, 8, 9, | 1, 2, 21, |
| | | | | | | 10, 11, 12, | 22 |
| | | | | | | 13, 15, 17, | |
| | | | | | | 18, 19, 20, | |
| | | | | | | 21, 22, 23 | |
| AS19 | 1 | 2, 6, 14, 16 | | | 3, 5 | 1, 4, 7, 8, 9, | 1, 2, 21, |
| | | | | | | 10, 11, 12, | 22 |
| | | | | | | 13, 15, 17, | |
| | | | | | | 18, 19, 20, | |
| | | | | | | 21, 22, 23 | |
| AS20 | 1 | 2, 14, 16 | | | 3, 6 | 1, 4, 5, 7, 8, | 1, 2, 21, |
| | | | | | | 9, 10, 11, | 22 |
| | | | | | | 12, 13, 15, | |
| | | | | | | 17, 18, 19, | |
| | | | | | | 20, 21, 22, | |
| | | | | | | 23 | |
| AS21 | 1 | 2, 6, 14, 16 | | | 3, 7 | 1, 4, 5, 8, 9, | 1, 2, 21, |
| | | | | | | 10, 11, 12, | 22 |
| | | | | | | 13, 15, 17, | |
| | | | | | | 18, 19, 20, | |
| | | | | | | 21, 22, 23 | |
| AS22 | 1 | At least one | None | 3 | At least | At least one | 1, 2, 21, |
| | | of the | | | one of | of the | and/or 22 |
| | | remaining | | | the | remaining | |
| | | positions | | | remaining | positions | |
| | | | | | positions | | |
| AS23 | 1 | At least one | None | 3, 5 | At least | At least one | 1, 2, 21, |
| | | among the | | | one | among the | and/or 22 |
| | | remaining | | | among | remaining | |
| | | positions | | | the | positions | |
| | | | | | remaining | | |
| | | | | | positions | | |
| AS24 | 1 | At least one | None | 3, 6 | At least | At least one | 1, 2, 21, |
| | | among the | | | one | among the | and/or 22 |
| | | remaining | | | among | remaining | |
| | | positions | | | the | positions | |
| | | | | | remaining | | |
| | | | | | positions | | |
| AS25 | 1 | At least one | None | 3, 7 | At least | At least one | 1, 2, 21, |
| | | among the | | | one | among the | and/or 22 |
| | | remaining | | | among | remaining | |
| | | positions | | | the | positions | |
| | | | | | remaining | | |
| | | | | | positions | | |

In some embodiments, the antisense strand having 23 nucleotides in length has the modification pattern of AS1. In some embodiments, the antisense strand having 23 nucleotides in length has the modification pattern of AS2. In some embodiments, the antisense strand having 23 nucleotides in length has the modification pattern of AS3. In some embodiments, the antisense strand having 23 nucleotides in length has the modification pattern of AS4. In some embodiments, the antisense strand having 23 nucleotides in length has the modification pattern of AS5. In some embodiments, the antisense strand having 23 nucleotides in length has the modification pattern of AS6. In some embodiments, the antisense strand having 23 nucleotides in length has the modification pattern of AS7. In some embodiments, the antisense strand having 23 nucleotides in length has the modification pattern of AS8. In some embodiments, the antisense strand having 23 nucleotides in length has the modification pattern of AS9. In some embodiments, the antisense strand having 23 nucleotides in length has the modification pattern of AS10. In some embodiments, the antisense strand having 23 nucleotides in length has the modification pattern of AS11. In some embodiments, the antisense strand having 23 nucleotides in length has the modification pattern of AS12. In some embodiments, the antisense strand having 23 nucleotides in length has the modification pattern of AS13. In some embodiments, the antisense strand having 23 nucleotides in length has the modification pattern of AS14. In some embodiments, the antisense strand having 23 nucleotides in length has the modification pattern of AS15. In some embodiments, the antisense strand having 23 nucleotides in length has the modification pattern of AS16. In some embodiments, the antisense strand having 23 nucleotides in length has the modification pattern of AS17. In some embodiments, the antisense strand having 23 nucleotides in length has the modification pattern of AS18. In some embodiments, the antisense strand having 23 nucleotides in length has the modification pattern of AS19. In some embodiments, the antisense strand having 23 nucleotides in length has the modification pattern of AS20. In some embodiments, the antisense strand having 23 nucleotides in length has the modification pattern of AS21. In some embodiments, the antisense strand having 23 nucleotides in length has the modification pattern of AS22. In some embodiments, the antisense strand having 23 nucleotides in length has the modification pattern of AS23. In some embodiments, the antisense strand having 23 nucleotides in length has the modification pattern of AS24. In some embodiments, the antisense strand having 23 nucleotides in length has the modification pattern of AS25. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises a nucleotide sequence selected from SEQ ID NOs: 777-792. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 777. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 778. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 779. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 780. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 781. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 782. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 783. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 784. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 785. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 786. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 787. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 788. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 789. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 790. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 791. In some embodiments, the antisense strand has a modification pattern of AS1, AS2, AS3, AS4, AS5, AS6, AS7, AS8, AS9, AS10, AS11, AS12, AS13, AS14, AS15, AS16, AS17, AS18, AS19, AS20, AS21, AS22, AS23, AS24, or AS25; and comprises SEQ ID NO: 792. In some embodiments, the antisense strand has a modification pattern of AS1; and comprises SEQ ID NO: 777. In some embodiments, the antisense strand has a modification pattern of AS2; and comprises SEQ ID NO: 777. In some embodiments, the antisense strand has a modification pattern of AS3; and comprises SEQ ID NO: 777. In some embodiments, the antisense strand has a modification pattern of AS4; and comprises SEQ ID NO: 777. In some embodiments, the antisense strand has a modification pattern of AS5; and comprises SEQ ID NO: 777. In some embodiments, the antisense strand has a modification pattern of AS6; and comprises SEQ ID NO: 777. In some embodiments, the antisense strand has a modification pattern of AS7; and comprises SEQ ID NO: 777. In some embodiments, the antisense strand has a modification pattern of AS8; and comprises SEQ ID NO: 777. In some embodiments, the antisense strand has a modification pattern of AS9; and comprises SEQ ID NO: 777. In some embodiments, the antisense strand has a modification pattern of AS10; and comprises SEQ ID NO: 777. In some embodiments, the antisense strand has a modification pattern of AS 11; and comprises SEQ ID NO: 777. In some embodiments, the antisense strand has a modification pattern of AS12; and comprises SEQ ID NO: 777. In some embodiments, the antisense strand has a modification pattern of AS13; and comprises SEQ ID NO: 777. In some embodiments, the antisense strand has a modification pattern of AS14; and comprises SEQ ID NO: 777. In some embodiments, the antisense strand has a modification pattern of AS15; and comprises SEQ ID NO: 777. In some embodiments, the antisense strand has a modification pattern of AS16; and comprises SEQ ID NO: 777. In some embodiments, the antisense strand has a modification pattern of AS17; and comprises SEQ ID NO: 777. In some embodiments, the antisense strand has a modification pattern of AS18; and comprises SEQ ID NO: 777. In some embodiments, the antisense strand has a modification pattern of AS19; and comprises SEQ ID NO: 777. In some embodiments, the antisense strand has a modification pattern of AS20; and comprises SEQ ID NO: 777. In some embodiments, the antisense strand has a modification pattern of AS21; and comprises SEQ ID NO: 777. In some embodiments, the antisense strand has a modification pattern of AS22; and comprises SEQ ID NO: 777. In some embodiments, the antisense strand has a modification pattern of AS23; and comprises SEQ ID NO: 777.

dsRNA Modification Pattern

In an aspect, the dsRNA as described herein includes a sense strand of Formula (I) as described herein and an antisense strand of Formula (II) as described herein. The sense strand and the antisense strand form a duplex. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand Formula (I) including 2′-MOE modifications at X₁, X₂, X₂₀ and X₂₁; and
(ii) an antisense strand Formula (II) including a 5′-(E)-VP-2′-OMe modification at X_{1′}. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand Formula (I) including:
   (a) 2′-MOE modifications at X₁, X₂, X₂₀ and X₂₁; and
   (b) 3′-PS modifications at X₁ and X₂, and
(ii) an antisense strand Formula (II) including:
   (a) a 5′-(E)-VP-2′-OMe modification at X_{1′}; and
   (b) 3′-PS modifications at X_{1′}, X_{2′}, X_{21′}, and X_{22′}. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand Formula (I) including:
   (a) 2′-MOE modifications at X₁, X₂, X₂₀ and X₂₁; and
   (b) 3′-PS modifications at X₁, X₂, X₁₉, and X₂₀,
   and
(ii) an antisense strand Formula (II) including:
   (a) a 5′-(E)-VP-2′-OMe modification at X_{1′}; and
   (b) 3′-PS modifications at X_{1′}, X_{2′}, X_{21′}, and X_{22′}. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand Formula (I) including:
   (a) 2′-MOE modifications at X₁, X₂, X₂₀ and X₂₁;
   (b) 3′-PS modifications at X₁, X₂, X₁₉, and/or X₂₀; and
   (c) one selected from:
   (c1) 2′-F modifications at X₇, X₉, X₁₀ and X₁₁; and 2′-OMe modifications in the remaining nucleotides in the sense strand, and
   (c2) 2′-F modifications at X₇, X₉, and X₁₀; 2′-deoxy modification at X₁₁; and 2′-OMe modifications in the remaining nucleotides in the sense strand,
   and
(ii) an antisense strand Formula (II) including:
   (a) a 5′-(E)-VP-2′-OMe modification at X_{1′};
   (b) 3′-PS modifications at X_{1′}, X_{2′}, X_{21′}, and X_{22′}; and
   (c) one selected from:
   (c1) 2′-F modifications at X_{2′}, X_{6′}, X_{14′} and X_{16′}; and 2′-OMe modifications in the remaining nucleotides in the antisense strand,
   (c2) 2′-F modifications at X_{2′}, X_{6′}, X_{14′} and X_{16′}; TNA at X_{3′}; and 2′-OMe modifications in the remaining nucleotides in the antisense strand,
   (c3) 2′-F modifications at X_{2′}, X_{6′}, X_{14′} and X_{16′}; TNA, GNA or 2′-deoxy modification at X_{5′}; and 2′-OMe modifications in the remaining nucleotides in the antisense strand,
   (c4) 2′-F modifications at X_{2′}, X_{14′} and X_{16′}; TNA, GNA or 2′-deoxy modification at X_{6′}; and 2′-OMe modifications in the remaining nucleotides in the antisense strand, and
   (c5) 2′-F modifications at X_{2′}, X_{6′}, X_{14′} and X_{16′}; TNA, GNA or 2′-deoxy modification at X_{7′}; and 2′-OMe modifications in the remaining nucleotides in the antisense strand. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand of Formula (I′) including TNAs at X₁, X₂, X₂₀ and X₂₁; and
(ii) an antisense strand of Formula (II) including a 5′-(E)-VP-2′-OMe modification at X_{1′}. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand of Formula (I′) including:
   (a) TNAs at X₁, X₂, X₂₀ and X₂₁; and
   (b) 3′-PS modifications at X₁ and X₂,
   and
(ii) an antisense strand of Formula (II) including:
   (a) a 5′-(E)-VP-2′-OMe modification at X_{1′}; and
   (b) 3′-PS modifications at X_{1′}, X_{2′}, X_{21′}, and X_{22′}. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand of Formula (I′) including:
   (a) TNAs at X₁, X₂, X₂₀ and X₂₁; and
   (b) 3′-PS modifications at X₁, X₂, X₁₉, and X₂₀,
   and
(ii) an antisense strand of Formula (II) including:
   (a) a 5′-(E)-VP-2′-OMe modification at X_{1′}; and
   (b) 3′-PS modifications at X_{1′}, X_{2′}, X_{21′}, and X_{22′}. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand of Formula (I′) including:
   (a) TNAs at X₁, X₂, X₂₀ and X₂₁;
   (b) 3′-PS modifications at X₁, X₂, X₁₉, and/or X₂₀; and
   (c) one selected from:
   (c1) 2′-F modifications at X₇, X₉, X₁₀ and X₁₁; and 2′-OMe modifications in the remaining nucleotides in the sense strand, and
   (c2) 2′-F modifications at X₇, X₉, and X₁₀; 2′-deoxy modification at X₁₁; and 2′-OMe modifications in the remaining nucleotides in the sense strand,
   and
(ii) an antisense strand of Formula (II) including:
   (a) a 5′-(E)-VP-2′-OMe modification at X_{1′};
   (b) 3′-PS modifications at X_{1′}, X_{2′}, X_{21′}, and X_{22′}; and
   (c) one selected from:
   (c1) 2′-F modifications at X_{2′}, X_{6′}, X_{14′} and X_{16′}; and 2′-OMe modifications in the remaining nucleotides in the antisense strand,
   (c2) 2′-F modifications at X_{2′}, X_{6′}, X_{14′} and X_{16′}; TNA at X_{3′}; and 2′-OMe modifications in the remaining nucleotides in the antisense strand,
   (c3) 2′-F modifications at X_{2′}, X_{6′}, X_{14′} and X_{16′}; TNA, GNA or 2′-deoxy modification at X₅; and 2′-OMe modifications in the remaining nucleotides in the antisense strand,
   (c4) 2′-F modifications at X_{2′}, X_{14′} and X_{16′}; TNA, GNA or 2′-deoxy modification at X_{6′}; and 2′-OMe modifications in the remaining nucleotides in the antisense strand, and
   (c5) 2′-F modifications at X_{2′}, X_{6′}, X_{14′} and X_{16′}; TNA, GNA or 2′-deoxy modification at X_{7′}; and 2′-OMe modifications in the remaining nucleotides in the antisense strand. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In certain aspects, the dsRNA as described herein includes a sense strand having 21 nucleotides in length and an antisense strand having 23 nucleotides.

In certain aspects, the dsRNA as described herein includes a sense strand having 21 nucleotides in length and including 2′-MOE modifications at the 1st, 2nd, 20th and 21st nucleotides from the 5′ end of the sense strand.

In certain aspects, the dsRNA as described herein includes antisense strand having 23 nucleotides in length and including a 5′-(E)-VP-2′-OMe modification at the 1st position from 5′ end of the antisense strand.

In some embodiments, the dsRNA includes:
(i) a sense strand having 21 nucleotides in length and including:
   (a) 2′-MOE modifications at the 1st, 2nd, 20th and 21st nucleotides from the 5′ end of the sense strand, and
   (b) 3′-PS modifications at the 1st and 2nd nucleotides from 5′ end of the sense strand, and
(ii) an antisense strand having 23 nucleotides in length and including:
   (a) a 5′-(E)-VP-2′-OMe modification at the 1st position from 5′ end of the antisense strand, and
   (b) 3′-PS modifications at the 1st, 2nd, 21st, and 22nd nucleotides from 5′ end of the antisense strand. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand having 21 nucleotides in length and including:
   (a) 2′-MOE modifications at the 1st, 2nd, 20th and 21st nucleotides from the 5′ end of the sense strand, and
   (b) 3′-PS modifications at the 1st, 2nd, 19th, and 20th nucleotides from 5′ end of the sense strand,
   and
(ii) an antisense strand having 23 nucleotides in length and including:
   (a) a 5′-(E)-VP-2′-OMe modification at the 1^{st} position from 5′ end of the antisense strand, and
   (b) 3′-PS modifications at the 1^{st}, 2^{nd}, 21^{st}, and 22^{nd} nucleotides from 5′ end of the antisense strand. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand having 21 nucleotides in length and including:
   (a) 2′-MOE modifications at the 1st, 2nd, 20th and 21st nucleotides from the 5′ end of the sense strand,
   (b) 3′-PS modifications at the 1st, 2nd, 19th, and/or 20th nucleotides from 5′ end of the sense strand, and
   (c) one selected from:
   (c1) 2′-F modifications at 7th, 9th, 10th, and 11th nucleotides; and 2′-OMe modifications in the remaining nucleotides in the sense strand, and
   (c2) 2′-F modifications at 7th, 9th, and 10th nucleotides; 2′-deoxy modification at 11th nucleotide; and 2′-OMe modifications in the remaining nucleotides in the sense strand,
   and
(ii) an antisense strand having 23 nucleotides in length and including:
   (a) a 5′-(E)-VP-2′-OMe modification at the 1^{st} position from 5′ end of the antisense strand,
   (b) 3′-PS modifications at the 1st, 2nd, 21st, and 22nd nucleotides from 5′ end of the antisense strand, and
   (c) one selected from:
   (c1) 2′-F modifications at the 2nd, 6th, 14th, and 16th nucleotides from 5′ end; and 2′-OMe modifications in the remaining nucleotides in the antisense strand,
   (c2) 2′-F modifications at the 2nd, 6th, 14th, and 16th nucleotides from 5′ end; TNA at the 3rd nucleotide from 5′ end; and 2′-OMe modifications in the remaining nucleotides in the antisense strand,
   (c3) 2′-F modifications at the 2nd, 6th, 14th, and 16th nucleotides from 5′ end; TNA, GNA or 2′-deoxy modification at the 5th nucleotide from 5′ end; and 2′-OMe modifications in the remaining nucleotides in the antisense strand,
   (c4) 2′-F modifications at the 2nd, 14th, and 16th nucleotides from 5′ end; TNA, GNA or 2′-deoxy modification at the 6th nucleotide from 5′ end; and 2′-OMe modifications in the remaining nucleotides in the antisense strand, and
   (c5) 2′-F modifications at the 2nd, 6th, 14th, and 16th nucleotides from 5′ end; TNA, GNA or 2′-deoxy modification at the 7th nucleotide from 5′ end; and 2′-OMe modifications in the remaining nucleotides in the antisense strand. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand having 21 nucleotides in length and including:
   (a) TNAs at the 1st, 2nd, 20th and 21st nucleotides from the 5′ end, and
   (b) 3′-PS modifications at the 1st and 2nd nucleotides from the 5′ end, and
(ii) an antisense strand having 23 nucleotides in length and including:
   (a) a 5′-(E)-VP-2′-OMe modification at the 1st position from the 5′ end, and
   (b) 3′-PS modifications at the 1st, 2nd, 21st, and 22nd nucleotides from the 5′ end. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand having 21 nucleotides in length and including:
   (a) TNAs at the 1st, 2nd, 20th and 21st nucleotides from the 5′ end, and
   (b) 3′-PS modifications at the 1st, 2nd, 19th, and 20th nucleotides from the 5′ end,
   and
(ii) an antisense strand having 23 nucleotides in length and including:
   (a) a 5′-(E)-VP-2′-OMe modification at the 1^{st} position from the 5′ end, and
   (b) 3′-PS modifications at the 1st, 2nd, 21st, and 22nd nucleotides from the 5′ end. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand having 21 nucleotides in length and including:
   (a) TNAs at the 1st, 2nd, 20th and 21st nucleotides from the 5′ end,
   (b) 3′-PS modifications at the 1st, 2nd, 19th, and/or 20th nucleotides from the 5′ end, and
   (c) one selected from:
   (c1) 2′-F modifications at the 7th, 9th, 10th, and 11th nucleotides from the 5′ end; and 2′-OMe modifications in the remaining nucleotides in the sense strand, and
   (c2) 2′-F modifications at the 7th, 9th, and 10th nucleotides from the 5′ end; 2′-deoxy modification at the 11th nucleotide from the 5′ end; and 2′-OMe modifications in the remaining nucleotides in the sense strand,
   and
(ii) an antisense strand having 23 nucleotides in length and including:
   (a) a 5′-(E)-VP-2′-OMe modification at the 1^{st} position from the 5′ end,
   (b) 3′-PS modifications at the 1st, 2nd, 21st, and 22nd nucleotides from the 5′ end, and
   (c) one selected from:
   (c1) 2′-F modifications at the 2nd, 6th, 14th, and 16th nucleotides from the 5′ end; and 2′-OMe modifications in the remaining nucleotides in the antisense strand,
   (c2) 2′-F modifications at the 2nd, 6th, 14th, and 16th nucleotides from the 5′ end; TNA at the 3rd nucleotide from the 5′ end; and 2′-OMe modifications in the remaining nucleotides in the antisense strand,
   (c3) 2′-F modifications at the 2nd, 6th, 14th, and 16th nucleotides from the 5′ end; TNA, GNA or 2′-deoxy modification at the 5th nucleotide from the 5′ end; and 2′-OMe modifications in the remaining nucleotides in the antisense strand,
   (c4) 2′-F modifications at the 2nd, 14th, and 16th nucleotides from the 5′ end; TNA, GNA or 2′-deoxy modification at the 6th nucleotide from the 5′ end; and 2′-OMe modifications in the remaining nucleotides in the antisense strand, and
   (c5) 2′-F modifications at the 2nd, 6th, 14th, and 16th nucleotides from the 5′ end; TNA, GNA or 2′-deoxy modification at the 7th nucleotide from the 5′ end; and 2′-OMe modifications in the remaining nucleotides in the antisense strand. In some embodiments, the sense strand comprises a sequence selected from SEQ ID NO: 761-776; and the antisense sequence comprises a sequence selected from SEQ ID NO: 777-792. In some embodiments, the sense strand comprises a sequence of SEQ ID NO: 761, and the antisense sequence comprises a sequence of SEQ ID NO: 777.

In some embodiments, the dsRNA includes:
(i) a sense strand that has a modification pattern of SS1, SS2, SS3, SS4, SS5, SS6, SS7, SS8, SS9, SS10, or SS11 and comprises a sequence of SEQ ID NO: 761; and
(ii) an antisense strand that has a modification pattern of AS1, AS2, AS3, AS4, AS5, AS6, AS7, AS8, AS9, AS10, ASH, AS12, AS13, AS14, AS15, AS16, AS17, AS18, AS19, AS20, AS21, AS22, AS23, AS24, or AS25 and comprises a sequence of SEQ ID NO: 777.

In some embodiments, the dsRNA includes:
a sense strand that has a modification pattern of SS10 or SS11 and comprises a sequence of SEQ ID NO: 761; and
an antisense strand that has a modification pattern of AS23, AS24, or AS25 and comprises a sequence of SEQ ID NO: 777.

Exemplary dsRNAs (siRNA) with modified nucleotides, including sense strands and antisense strands targeting the above indicated PCSK9 mRNA, are shown in Table 5.

TABLE 5
Examples of modified nucleotide sequence of PCSK9 siRNA

| | Site | | | | |
|---|---|---|---|---|---|
| | of | | SEQ | | SEQ |
| siRNA | mRNA | | ID | | ID |
| No. | target | Sense strand | NO. | Antisense strand | NO. |
| M1 | 3551 | C004p001U004p001A004pG0 | 793 | A004p001C007p001A004pA00 | 846 |
| | | 04pA004pC004pC007pU004p | | 7pA007pA007pG004pC007pA0 | |
| | | G007pU004pT002pU004pU00 | | 04pA007pA004pA007pC004pA | |
| | | 4pG004pC004pU004pU004pU | | 007pG004pG007pU004pC007p | |
| | | 004pU004pG004pU004 | | U004pA004pG004p001A004p0 | |
| | | | | 01A004 | |
| M2 | 3551 | C004p001U004p001A004pG0 | 794 | A004p001C007p001A004pA00 | 847 |
| | | 04pA004pC004pC007pU004p | | 4pA004pA007pG004pC004pA0 | |
| | | G007pU007pT002pU004pU00 | | 04pA004pA004pA004pC004pA | |
| | | 4pG004pC004pU004pU004pU | | 007pG004pG007pU004pC004p | |
| | | 004pU004pG004pU004 | | U004pA004pG004p001A004p0 | |
| | | | | 01A004 | |
| M3 | 3551 | C004p001U004p001A004pG0 | 795 | X033A1027p001C007p001A00 | 848 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG004pU004 | | C004pU004pA004pG004p001A | |
| | | | | 004p001A004 | |
| M4 | 3551 | C005p001T005p001A004pG0 | 796 | X033A1027p001C007p001A00 | 849 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG005pT005 | | C004pU004pA004pG004p001A | |
| | | | | 004p001A004 | |
| M5 | 3551 | C005p001T005p001A004pG0 | 797 | A004p001C007p001A004pA00 | 850 |
| | | 04pA004pC004pC007pU004p | | 4pA004pA007pG004pC004pA0 | |
| | | G007pU007pT002pU004pU00 | | 04pA004pA004pA004pC004pA | |
| | | 4pG004pC004pU004pU004pU | | 007pG004pG007pU004pC004p | |
| | | 004pU004pG005pT005 | | U004pA004pG004p001A004p0 | |
| | | | | 01A004 | |
| M6 | 3551 | C005p001T005p001A004pG0 | 798 | X033U1027p001C007p001A00 | 851 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG005pA005 | | C004pU004pA004pG004p001A | |
| | | | | 005p001A005 | |
| M7 | 3551 | C004p001U004p001A004pG0 | 799 | A004p001C007p001A004pA00 | 852 |
| | | 04pA004pC004pC007pU004p | | 7pA007pA007pG004pC007pA0 | |
| | | G007pU004pT002pU004pU00 | | 04pA007pA004pA007pC004pA | |
| | | 4pG004pC004pU004pU004pU | | 007pG004pG007pU004pC007p | |
| | | 004pU004pG004pU004 | | U004pA004pG004p001A004p0 | |
| | | | | 01A004 | |
| M8 | 3551 | C005p001T005p001A004pG0 | 800 | X033A1027p001C007p001A00 | 853 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG005pT005 | | C004pU004pA004pG004p001A | |
| | | | | 004p001A004 | |
| M9 | 3551 | C005p001T005p001A004pG0 | 801 | A004p001C007p001A004pA00 | 854 |
| | | 04pA004pC004pC007pU004p | | 4pA004pA007pG004pC004pA0 | |
| | | G007pU007pT002pU004pU00 | | 04pA004pA004pA004pC004pA | |
| | | 4pG004pC004pU004pU004pU | | 007pG004pG007pU004pC004p | |
| | | 004pU004pG005pT005 | | U004pA004pG004p001A004p0 | |
| | | | | 01A004 | |
| M10 | 3551 | C004p001U004p001A004pG0 | 802 | X033A1027p001C007p001A00 | 855 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG004pU004 | | C004pU004pA004pG004p001A | |
| | | | | 004p001A004 | |
| M11 | 3551 | C005p001T005p001A004pG0 | 803 | X033U1027p001C007p001A00 | 856 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG005pA005 | | C004pU004pA004pG004p001A | |
| | | | | 004p001A004 | |
| M12 | 3551 | C005p001T005p001A004pG0 | 804 | X033U1027p001C007p001A00 | 857 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001A00 | | C004pU004pA004pG004p001A | |
| | | 5 | | 004p001A004 | |
| M13 | 3551 | C005p001T005p001A004pG0 | 805 | X033A1027p001C007p001A00 | 858 |
| | | 04pA004pC004pC007pU004p | | 4pA004pA004pA007pG004pC0 | |
| | | G007pU007pU007pU004pU0 | | 04pA004pA004pA004pA004pC | |
| | | 04pG004pC004pU004pU004p | | 004pA007pG004pG007pU004p | |
| | | U004pU004pG005pT005 | | C004pU004pA004pG004p001A | |
| | | | | 004p001A004 | |
| M14 | 3551 | C005p001T005p001A004pG0 | 806 | X033A1027p001C007p001A00 | 859 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | | | 004p001A004 | |
| M15 | 3551 | C005p001T005p001A004pG0 | 807 | X033A1027p001C007p001A00 | 860 |
| | | 04pA004pC004pC007pU004p | | 4pA004pA004pA007pG004pC0 | |
| | | G007pU007pU007pU004pU0 | | 04pA004pA004pA004pA004pC | |
| | | 04pG004pC004pU004pU004p | | 004pA007pG004pG007pU004p | |
| | | U004pU004p001G005p001TO | | C004pU004pA004pG004p001A | |
| | | 05 | | 004p001A004 | |
| M16 | 3551 | C005p001T005p001A004pG0 | 808 | A004p001C007p001A004pA00 | 861 |
| | | 04pA004pC004pC007pU004p | | 4pA004pA007pG004pC004pA0 | |
| | | G007pU007pT002pU004pU00 | | 04pA004pA004pA004pC004pA | |
| | | 4pG004pC004pU004pU004pU | | 007pG004pG007pU004pC004p | |
| | | 004pU004p001G005p001T005 | | U004pA004pG004p001A004p0 | |
| | | | | 01A004 | |
| M17 | 3551 | C005p001T005p001A004pG0 | 809 | A004p001C007p001A004pA00 | 862 |
| | | 04pA004pC004pC007pU004p | | 4pA004pA007pG004pC004pA0 | |
| | | G007pU007pU007pU004pU0 | | 04pA004pA004pA004pC004pA | |
| | | 04pG004pC004pU004pU004p | | 007pG004pG007pU004pC004p | |
| | | U004pU004p001G005p001TO | | U004pA004pG004p001A004p0 | |
| | | 05 | | 01A004 | |
| M18 | 3551 | C005p001T005p001A004pG0 | 810 | X033A1027p001C007p001A04 | 863 |
| | | 04pA004pC004pC007pU004p | | 2pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG005pT005 | | C004pU004pA004pG004p001A | |
| | | | | 004p001A004 | |
| M19 | 3551 | C005p001T005p001A004pG0 | 811 | X033A1027p001C007p001A04 | 864 |
| | | 04pA004pC004pC007pU004p | | 2pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | | | 004p001A004 | |
| M20 | 3551 | C042p001U042p001A004pG0 | 812 | X033A1027p001C007p001A00 | 865 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG042pU042 | | C004pU004pA004pG004p001A | |
| | | | | 004p001A004 | |
| M21 | 3551 | C042p001U042p001A004pG0 | 813 | X033A1027p001C007p001A00 | 866 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G042p001U04 | | C004pU004pA004pG004p001A | |
| | | 2 | | 004p001A004 | |
| M22 | 3551 | C042p001U042p001A004pG0 | 814 | X033A1027p001C007p001A04 | 867 |
| | | 04pA004pC004pC007pU004p | | 2pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG042pU042 | | C004pU004pA004pG004p001A | |
| | | | | 004p001A004 | |
| M23 | 3551 | C042p001U042p001A004pG0 | 815 | X033A1027p001C007p001A04 | 868 |
| | | 04pA004pC004pC007pU004p | | 2pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G042p001U04 | | C004pU004pA004pG004p001A | |
| | | 2 | | 004p001A004 | |
| M24 | 3492 | C005p001A005p001A004pG0 | 816 | X033U1027p001A007p001A00 | 869 |
| | | 04pC004pA004pG007pA004p | | 4pA004pA004pG007pA004pU0 | |
| | | C007pA007pU007pU004pU00 | | 04pA004pA004pA004pU004pG | |
| | | 4pA004pU004pC004pU004pU | | 004pU007pC004pU007pG004p | |
| | | 004pU004PT005pA005 | | C004pU004pU004pG004p001C | |
| | | | | 004p001U004 | |
| M25 | 3543 | C005p001A005p001A004pC0 | 817 | X033A1027p001A007p001A00 | 870 |
| | | 04pU004pU004pU007pU004p | | 4pA004pC004pA007pG004pG0 | |
| | | C007pU007pA007pG004pA00 | | 04pU004pC004pU004pA004pG | |
| | | 4pC004pC004pU004pG004pU | | 004pA007pA004pA007pA004p | |
| | | 004pU004pT005pT005 | | G004pU004pU004pG004p001G | |
| | | | | 004p001C004 | |
| M26 | 3564 | G005p001C005p001U004pU0 | 818 | X033A1027p001U007p001A00 | 871 |
| | | 04pU004pU004pG007pU004p | | 4pU004pC004pU007pU004pC0 | |
| | | A007pA007pC007pU004pU00 | | 04pA004pA004pG004pU004pU | |
| | | 4pG004pA004pA004pG004p | | 004pA007pC004pA007pA004p | |
| | | A004pU004pA005pT005 | | A004pA004pG004pC004p001A | |
| | | | | 004p001A004 | |
| M27 | 713 | T005p001T005p001G004pA0 | 819 | X033U1027p001U007p001C00 | 872 |
| | | 04pA004pG004pU007pU004p | | 4pG004pA004pC007pA004pU0 | |
| | | G007pC007pC007pC004pC00 | | 04pG004pG004pG004pG004pC | |
| | | 4pA004pU004pG004pU004pC | | 004pA007pA004pC007pU004p | |
| | | 004pG004pA005pA005 | | U004pC004pA004pA004p001G | |
| | | | | 004p001G004 | |
| M28 | 1102 | C005p001C005p001U004pC0 | 820 | X033A1027p001U007p001A00 | 873 |
| | | 04pA004pU004pA007pG004p | | 4pA004pA004pC007pU004pC0 | |
| | | G007pC007pC007pU004pG00 | | 04pC004pA004pG004pG004pC | |
| | | 4pG004pA004pG004pU004p | | 004pC007pU004pA007pU004p | |
| | | U004pU004pA005pT005 | | G004pA004pG004pG004p001G | |
| | | | | 004p001U004 | |
| M29 | 3251 | A005p001A005p001G004pCO | 821 | X033A1027p001A007p001G00 | 874 |
| | | 04pC004pA004pA007pG004p | | 4pU004pA004pA007pG004pA0 | |
| | | C007pC007pU007pC004pU00 | | 04pA004pG004pA004pG004pG | |
| | | 4pU004pC004pU004pU004pA | | 004pC007pU004pU007pG004p | |
| | | 004pC004pT005pT005 | | G004pC004p001U004p001U00 | |
| | | | | 4pC004pA004 | |
| M30 | 3252 | A005p001G005p001C004pC0 | 822 | X033U1027p001A007p001A00 | 875 |
| | | 04pA004pA004pG007pC004p | | 4pG004pU004pA007pA004pG0 | |
| | | C007pU007pC007pU004pU00 | | 04pA004pA004pG004pA004pG | |
| | | 4pC004pU004pU004pA004pC | | 004pG007pC004pU007pU004p | |
| | | 004pU004pT005pA005 | | G004pG004pC004pU004p001U | |
| | | | | 004p001C004 | |
| M31 | 3547 | T005p001T005p001U004pU0 | 823 | X033A1027p001A007p001G00 | 876 |
| | | 04pC004pU004pA007pG004p | | 4pC004pA004pA007pA004pA0 | |
| | | A007pC007pC007pU004pG00 | | 04pC004pA004pG004pG004pU | |
| | | 4pU004pU004pU004pU004p | | 004pC007pU004pA007pG004p | |
| | | G004pC004pT005pT005 | | A004pA004p001A004p001A00 | |
| | | | | 4pG004pU004 | |
| M32 | 3553 | A005p001G005p001A004pC0 | 824 | X033U1027p001U007p001A00 | 877 |
| | | 04pC004pU004pG007pU004p | | 4pC004pA004pA007pA004pA0 | |
| | | U007pU007pU007pG004pC00 | | 04pG004pC004pA004pA004pA | |
| | | 4pU004pU004pU004pU004p | | 004pA007pC004pA007pG004p | |
| | | G004pU004pA005pA005 | | G004pU004pC004pU004p001A | |
| | | | | 004p001G004 | |
| M33 | 3568 | T005p001T005p001G004pU0 | 825 | X033A1027p001U007p001A00 | 878 |
| | | 04pA004pA004pC007pU004p | | 4pA004pA004pU007pA004pU0 | |
| | | U007pG007pA007pA004pG0 | | 04pC004pU004pU004pC004pA | |
| | | 04pA004pU004pA004pU004p | | 004pA007pG004pU007pU004p | |
| | | U004pU004pA005pT005 | | A004pC004p001A004p001A00 | |
| | | | | 4pA004pA004 | |
| M34 | 3559 | G005p001T005p001U004pU0 | 826 | X033U1027p001U007p001C00 | 879 |
| | | 04pU004pG004pC007pU004p | | 4pA004pA004pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004pA005pA005 | | A004pA004pA004pC004p001A | |
| | | | | 004p001G004 | |
| M35 | 3547 | T005p001T005p001U004pU0 | 827 | X033A1027p001A007p001G00 | 880 |
| | | 04pC004pU004pA004pG004p | | 4pC004pA004pA007pA004pA0 | |
| | | A007pC007pC007pU004pG00 | | 04pC004pA004pG004pG004pU | |
| | | 4pU004pU004pU004pU004p | | 004pC007pU004pA007pG004p | |
| | | G004pC004p001T005p001TO | | A004pA004p001A004p001A00 | |
| | | 05 | | 4pG004pU004 | |
| M36 | 3553 | A005p001G005p001A004pC0 | 828 | X033U1027p001U007p001A00 | 881 |
| | | 04pC004pU004pG007pU004p | | 4pC004pA004pA007pA004pA0 | |
| | | U007pU007pU007pG004pC00 | | 04pG004pC004pA004pA004pA | |
| | | 4pU004pU004pU004pU004p | | 004pA007pC004pA007pG004p | |
| | | G004pU004p001A005p001A0 | | G004pU004pC004pU004p001A | |
| | | 05 | | 004p001G004 | |
| M37 | 3568 | T005p001T005p001G004pU0 | 829 | X033A1027p001U007p001A00 | 882 |
| | | 04pA004pA004pC004pU004p | | 4pA004pA004pU007pA004pU0 | |
| | | U007pG007pA007pA004pG0 | | 04pC004pU004pU004pC004pA | |
| | | 04pA004pU004pA004pU004p | | 004pA007pG004pU007pU004p | |
| | | U004pU004p001A005p001TO | | A004pC004p001A004p001A00 | |
| | | 05 | | 4pA004pA004 | |
| M38 | 3559 | G005p001T005p001U004pU0 | 830 | X033U1027p001U007p001C00 | 883 |
| | | 04pU004pG004pC007pU004p | | 4pA004pA004pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M39 | 3559 | G005p001T005p001U004pU0 | 831 | X033U1027p001U007p001C00 | 884 |
| | | 04pU004pG004pC007pU004p | | 4pA004pA1016pG007pU004pU | |
| | | U007pU007pU007pG004pU0 | | 004pA004pC004pA004pA004p | |
| | | 04pA004pA004pC004pU004p | | A004pA007pG004pC007pA004 | |
| | | U004pG004p001A005p001A0 | | pA004pA004pA004pC004p001 | |
| | | 05 | | A004p001G004 | |
| M40 | 3559 | G005p001T005p001U004pU0 | 832 | X033U1027p001U007p001C00 | 885 |
| | | 04pU004pG004pC007pU004p | | 4pA004pA004pG1016pU004pU | |
| | | U007pU007pU007pG004pU0 | | 004pA004pC004pA004pA004p | |
| | | 04pA004pA004pC004pU004p | | A004pA007pG004pC007pA004 | |
| | | U004pG004p001A005p001A0 | | pA004pA004pA004pC004p001 | |
| | | 05 | | A004p001G004 | |
| M41 | 3559 | G005p001T005p001U004pU0 | 833 | X033U1027p001U007p001C00 | 886 |
| | | 04pU004pG004pC007pU004p | | 4pA004pA004pG007pU1016pU | |
| | | U007pU007pU007pG004pU0 | | 004pA004pC004pA004pA004p | |
| | | 04pA004pA004pC004pU004p | | A004pA007pG004pC007pA004 | |
| | | U004pG004p001A005p001A0 | | pA004pA004pA004pC004p001 | |
| | | 05 | | A004p001G004 | |
| M42 | 3559 | G005p001T005p001U004pU0 | 834 | X033U1027p001U007p001C00 | 887 |
| | | 04pU004pG004pC007pU004p | | 4pA004pA042pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M43 | 3559 | G005p001T005p001U004pU0 | 835 | X033U1027p001U007p001C00 | 888 |
| | | 04pU004pG004pC007pU004p | | 4pA004pA004pG042pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M44 | 3559 | G005p001T005p001U004pU0 | 836 | X033U1027p001U007p001C00 | 889 |
| | | 04pU004pG004pC007pU004p | | 4pA004pA004pG007pU042pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M45 | 3559 | G005p001T005p001U004pU0 | 837 | X033U1027p001U007p001C00 | 890 |
| | | 04pU004pG004pC007pU004p | | 4pA004pA002pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M46 | 3559 | G005p001T005p001U004pU0 | 838 | X033U1027p001U007p001C00 | 891 |
| | | 04pU004pG004pC007pU004p | | 4pA004pA004pG002pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M47 | 3559 | G005p001T005p001U004pU0 | 839 | X033U1027p001U007p001C00 | 892 |
| | | 04pU004pG004pC007pU004p | | 4pA004pA004pG007pT002pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M48 | 3559 | G005p001T005p001U004pU0 | 840 | X033U1027p001U007p001U00 | 893 |
| | | 04pU004pG004pC007pU004p | | 4pA004pA004pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pA004p001A005p001A0 | | A004pA004pA004pC004p001A | |
| | | 05 | | 004p001G004 | |
| M49 | 3559 | G005p001T005p001U004pU0 | 841 | X033U1027p001U007p001U00 | 894 |
| | | 04pU004pG004pC007pU004p | | 4pA004pA004pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M50 | 3559 | G005p001T005p001U004pU0 | 842 | X033U1027p001U007p001C04 | 895 |
| | | 04pU004pG004pC007pU004p | | 2pA004pA042pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M51 | 3559 | G005p001T005p001U004pU0 | 843 | X033U1027p001U007p001C04 | 896 |
| | | 04pU004pG004pC007pU004p | | 2pA004pA004pG042pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M52 | 3559 | G005p001T005p001U004pU0 | 844 | X033U1027p001U007p001C04 | 897 |
| | | 04pU004pG004pC007pU004p | | 2pA004pA004pG007pU042pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| M53 | 3559 | G005p001T005p001U004pU0 | 845 | X033U1027p001U007p001C00 | 898 |
| | | 04pU004pG004pC007pU004p | | 4pA004pA002pG007pT002pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05 | | 004p001G004 | |
| N49 | 3551 | A1039p001C005p001T005pA | 1013 | X033A1032p001C007p001A00 | 1019 |
| | | 004pG004pA004pC004pC007 | | 4pA004pA004pA007pG004pC0 | |
| | | pU004pG007pU007pT002pU0 | | 04pA004pA004pA004pA004pC | |
| | | 04pU004pG004pC004pU004p | | 004pA007pG004pG007pU004p | |
| | | U004pU004pU004p001G005p | | C004pU004pA004pG004p001A | |
| | | 001T005 | | 004p001A004 | |
| N50 | 3551 | A1039p001C005p001T005pA | 1014 | X033A1027p001C007p001A00 | 1020 |
| | | 004pG004pA004pC004pC007 | | 4pA004pA004pA007pG004pC0 | |
| | | pU004pG007pU007pT002pU0 | | 04pA004pA004pA004pA004pC | |
| | | 04pU004pG004pC004pU004p | | 004pA007pG004pG007pU004p | |
| | | U004pU004pU004pG005p001 | | C004pU004pA004pG004p001A | |
| | | T005p001A1039 | | 004p001A004 | |
| N60 | 3551 | C005p001T005p001A004pG0 | 1015 | X033A1027p001C007p001A04 | 1021 |
| | | 04pA004pC004pC007pU004p | | 2pA004pA042pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | | | 004p001A004 | |
| N61 | 3551 | C005p001T005p001A004pGO | 1016 | X033A1027p001C007p001A04 | 1022 |
| | | 04pA004pC004pC007pU004p | | 2pA004pA004pA042pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | | | 004p001A004 | |
| N62 | 3551 | C005p001T005p001A004pG0 | 1017 | X033A1027p001C007p001A04 | 1023 |
| | | 04pA004pC004pC007pU004p | | 2pA004pA004pA007pG042pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | | | 004p001A004 | |
| N63 | 3551 | X2171p001C005p001T005pA | 1018 | X033A1032p001C007p001A00 | 1024 |
| | | 004pG004pA004pC004pC007 | | 4pA004pA004pA007pG004pC0 | |
| | | pU004pG007pU007pT002pU0 | | 04pA004pA004pA004pA004pC | |
| | | 04pU004pG004pC004pU004p | | 004pA007pG004pG007pU004p | |
| | | U004pU004pU004p001G005p | | C004pU004pA004pG004p001A | |
| | | 001T005 | | 004p001A004 | |

Table A below shows codes in the nucleotide sequences in Table 5 and the following Tables in the disclosure.

TABLE A
p: phosphodiester linkage
p001: phosphorothioate (PS) linkage
SS: sense strand AS: antisense strand
A1039 or X2171: inverted abasic nucleotide

| | |
|---|---|
| A002: 2′-deoxy adenosine (dA) | G002: 2′-deoxy guanosine (dG) |
| A004: adenosine /2′-OMe | G004: guanosine /2′-OMe |
| A005: adenosine /2′-MOE | G005: guanosine /2′-MOE |
| A007: adenosine/2′-F | G007: guanosine/2′-F |
| A042: adenosine TNA | G042: guanosine TNA |
| A1016: adenosine GNA | G1016: guanosine GNA |
| X033A1027: Adenosine /5′-(E)-VP-2′-OMe | X033G1027: guanosine / 5′-(E)-VP-2′-OMe |
| X033A1032: Adenosine /5′-(E)-VP-2′-MOE | |
| C002: 2′-deoxy cytidine (dC) | T002: 2′-deoxy thymidine (dT) |
| C004: cytidine /2′-OMe | U004: uridine /2′-OMe |
| C005: 5-methyl-cytidine /2′-MOE | T005: ribothymidine (5-methyl uridine) /2′-MOE |
| C007: cytidine /2′-F | U007: uridine /2′-F |
| C042: cytidine TNA | U042: uridine TNA |
| C1016: cytosine GNA | U1016: uridine GNA |
| X033C1027: cytidine / 5′-(E)-VP-2′-OMe | X033U1027: uridine / 5′-(E)-VP-2′-OMe |

The sequences and sequence lists including modified nucleosides (e.g., RNA, RNA modified at a 2′-OH sugar moiety, or RNA modified at a nucleobase) in the disclosure (e.g., Tables 5-9) are indicated with codes defined in Table A unless otherwise indicated. Each code consists of a letter representing a type of the nucleobase, e.g., “A”, “G”, “C”, “U,” or “T” and a numeric code representing a type of modification on a sugar ring.

For example, if a nucleoside is coded as “T005”, it is meant by a RNA nucleoside including a 2′-MOE ribose sugar moiety and a thymine (or methylated uracil) as “T” indicates a thymine (or methylated uracil) nucleobase and “005” indicates a 2′-MOE substituent at 2′-OH position on the ribose sugar ring.

In particular example, if a nucleoside is coded as “C005”, it is meant by a RNA nucleoside including a 5-methylated cytosine and a 2′-MOE sugar moiety as “C” indicates a type of specific nucleobase that can exist in combination with a 2′-MOE sugar moiety and “005” indicates a 2′-MOE substituent at 2′-OH position on the ribose sugar ring.

In another example, if a nucleoside is coded as “A042”, it is meant by a RNA nucleoside including an adenine and a threofuranosyl sugar ring as “A” indicates a type of adenine and “042” indicates a threofuranosyl ring replacing the ribose sugar ring.

In some embodiments, the dsRNA includes a sense strand having 10 contiguous nucleotides differing by no more than 3 nucleotides from the nucleotide sequence selected from SEQ ID NOs: 793 to 845 and 1013 to 1018. In some embodiments, the dsRNA includes an antisense strand having 10 contiguous nucleotides differing by no more than 3 nucleotides from the nucleotide sequence of SEQ ID NOs: 846 to 898 and 1019 to 1024. In some embodiments, the dsRNA includes a sense strand having 11 contiguous nucleotides differing by no more than 3 nucleotides from the nucleotide sequence selected from SEQ ID NOs: 793 to 845 and 1013 to 1018. In some embodiments, the dsRNA includes an antisense strand having 11 contiguous nucleotides differing by no more than 3 nucleotides from the nucleotide sequence selected from SEQ ID NOs: 846 to 898 and 1019 to 1024. In some embodiments, the dsRNA includes a sense strand having 12 contiguous nucleotides differing by no more than 3 nucleotides from the nucleotide sequence selected from SEQ ID NOs: 793 to 845 and 1013 to 1018. In some embodiments, the dsRNA includes an antisense strand having 12 contiguous nucleotides differing by no more than 3 nucleotides from the nucleotide sequence selected from SEQ ID NOs: 846 to 898 and 1019 to 1024. In some embodiments, the dsRNA includes a sense strand having 13 contiguous nucleotides differing by no more than 3 nucleotides from the nucleotide sequence selected from SEQ ID NOs: 793 to 845 and 1013 to 1018. In some embodiments, the dsRNA includes an antisense strand having 13 contiguous nucleotides differing by no more than 3 nucleotides from the nucleotide sequence selected from SEQ ID NOs: 846 to 898 and 1019 to 1024. In some embodiments, the dsRNA includes a sense strand having 14 contiguous nucleotides differing by no more than 3 nucleotides from the nucleotide sequence selected from SEQ ID NOs: 793 to 845 and 1013 to 1018. In some embodiments, the dsRNA includes an antisense strand having 14 contiguous nucleotides differing by no more than 3 nucleotides from the nucleotide sequence selected from SEQ ID NOs: 846 to 898 and 1019 to 1024. In some embodiments, the dsRNA includes a sense strand having 15 contiguous nucleotides differing by no more than 3 nucleotides from the nucleotide sequence selected from SEQ ID NOs: 793 to 845 and 1013 to 1018. In some embodiments, the dsRNA includes an antisense strand having 15 contiguous nucleotides differing by no more than 3 nucleotides from the nucleotide sequence selected from SEQ ID NOs: 846 to 898 and 1019 to 1024. In some embodiments, the dsRNA includes a sense strand having 16 contiguous nucleotides differing by no more than 3 nucleotides from the nucleotide sequence selected from SEQ ID NOs: 793 to 845 and 1013 to 1018. In some embodiments, the dsRNA includes an antisense strand having 16 contiguous nucleotides differing by no more than 3 nucleotides from the nucleotide sequence selected from SEQ ID NOs: 846 to 898 and 1019 to 1024. In some embodiments, the dsRNA includes a sense strand having 17 contiguous nucleotides differing by no more than 3 nucleotides from the nucleotide sequence selected from SEQ ID NOs: 793 to 845 and 1013 to 1018. In some embodiments, the dsRNA includes an antisense strand having 17 contiguous nucleotides differing by no more than 3 nucleotides from the nucleotide sequence selected from SEQ ID NOs: 846 to 898 and 1019 to 1024. In some embodiments, the dsRNA includes a sense strand having 18 contiguous nucleotides differing by no more than 3 nucleotides from the nucleotide sequence selected from SEQ ID NOs: 793 to 845 and 1013 to 1018. In some embodiments, the dsRNA includes an antisense strand having 18 contiguous nucleotides differing by no more than 3 nucleotides from the nucleotide sequence selected from SEQ ID NOs: 846 to 898 and 1019 to 1024. In some embodiments, the dsRNA includes a sense strand having 19 contiguous nucleotides differing by no more than 3 nucleotides from the nucleotide sequence selected from SEQ ID NOs: 793 to 845 and 1013 to 1018. In some embodiments, the dsRNA includes an antisense strand having 19 contiguous nucleotides differing by no more than 3 nucleotides from the nucleotide sequence selected from SEQ ID NOs: 846 to 898 and 1019 to 1024. In some embodiments, the dsRNA includes a sense strand having 20 contiguous nucleotides differing by no more than 3 nucleotides from the nucleotide sequence selected from SEQ ID NOs: 793 to 845 and 1013 to 1018. In some embodiments, the dsRNA includes an antisense strand having 20 contiguous nucleotides differing by no more than 3 nucleotides from the nucleotide sequence selected from SEQ ID NOs: 846 to 898 and 1019 to 1024. In some embodiments, the dsRNA includes a sense strand having 21 contiguous nucleotides differing by no more than 3 nucleotides from the nucleotide sequence selected from SEQ ID NOs: 793 to 845 and 1013 to 1018. In some embodiments, the dsRNA includes an antisense strand having 21 contiguous nucleotides differing by no more than 3 nucleotides from the nucleotide sequence selected from SEQ ID NOs: 846 to 898 and 1019 to 1024.

In some embodiments, the dsRNA includes a sense strand having 10 contiguous nucleotides differing by no more than 2 nucleotides from the nucleotide sequence selected from SEQ ID NOs: 793 to 845 and 1013 to 1018. In some embodiments, the dsRNA includes an antisense strand having 10 contiguous nucleotides differing by no more than 2 nucleotides from the nucleotide sequence selected from SEQ ID NOs: 846 to 898 and 1019 to 1024. In some embodiments, the dsRNA includes a sense strand having 11 contiguous nucleotides differing by no more than 2 nucleotides from the nucleotide sequence selected from SEQ ID NOs: 793 to 845 and 1013 to 1018. In some embodiments, the dsRNA includes an antisense strand having 11 contiguous nucleotides differing by no more than 2 nucleotides from the nucleotide sequence selected from SEQ ID NOs: 846 to 898 and 1019 to 1024. In some embodiments, the dsRNA includes a sense strand having 12 contiguous nucleotides differing by no more than 2 nucleotides from the nucleotide sequence selected from SEQ ID NOs: 793 to 845 and 1013 to 1018. In some embodiments, the dsRNA includes an antisense strand having 12 contiguous nucleotides differing by no more than 2 nucleotides from the nucleotide sequence selected from SEQ ID NOs: 846 to 898 and 1019 to 1024. In some embodiments, the dsRNA includes a sense strand having 13 contiguous nucleotides differing by no more than 2 nucleotides from the nucleotide sequence selected from SEQ ID NOs: 793 to 845 and 1013 to 1018. In some embodiments, the dsRNA includes an antisense strand having 13 contiguous nucleotides differing by no more than 2 nucleotides from the nucleotide sequence selected from SEQ ID NOs: 846 to 898 and 1019 to 1024. In some embodiments, the dsRNA includes a sense strand having 14 contiguous nucleotides differing by no more than 2 nucleotides from the nucleotide sequence selected from SEQ ID NOs: 793 to 845 and 1013 to 1018. In some embodiments, the dsRNA includes an antisense strand having 14 contiguous nucleotides differing by no more than 2 nucleotides from the nucleotide sequence selected from SEQ ID NOs: 846 to 898 and 1019 to 1024. In some embodiments, the dsRNA includes a sense strand having 15 contiguous nucleotides differing by no more than 2 nucleotides from the nucleotide sequence selected from SEQ ID NOs: 793 to 845 and 1013 to 1018. In some embodiments, the dsRNA includes an antisense strand having 15 contiguous nucleotides differing by no more than 2 nucleotides from the nucleotide sequence selected from SEQ ID NOs: 846 to 898 and 1019 to 1024. In some embodiments, the dsRNA includes a sense strand having 16 contiguous nucleotides differing by no more than 2 nucleotides from the nucleotide sequence selected from SEQ ID NOs: 793 to 845 and 1013 to 1018. In some embodiments, the dsRNA includes an antisense strand having 16 contiguous nucleotides differing by no more than 2 nucleotides from the nucleotide sequence selected from SEQ ID NOs: 846 to 898 and 1019 to 1024. In some embodiments, the dsRNA includes a sense strand having 17 contiguous nucleotides differing by no more than 2 nucleotides from the nucleotide sequence selected from SEQ ID NOs: 793 to 845 and 1013 to 1018. In some embodiments, the dsRNA includes an antisense strand having 17 contiguous nucleotides differing by no more than 2 nucleotides from the nucleotide sequence selected from SEQ ID NOs: 846 to 898 and 1019 to 1024. In some embodiments, the dsRNA includes a sense strand having 18 contiguous nucleotides differing by no more than 2 nucleotides from the nucleotide sequence selected from SEQ ID NOs: 793 to 845 and 1013 to 1018. In some embodiments, the dsRNA includes an antisense strand having 18 contiguous nucleotides differing by no more than 2 nucleotides from the nucleotide sequence selected from SEQ ID NOs: 846 to 898 and 1019 to 1024. In some embodiments, the dsRNA includes a sense strand having 19 contiguous nucleotides differing by no more than 2 nucleotides from the nucleotide sequence selected from SEQ ID NOs: 793 to 845 and 1013 to 1018. In some embodiments, the dsRNA includes an antisense strand having 19 contiguous nucleotides differing by no more than 2 nucleotides from the nucleotide sequence selected from SEQ ID NOs: 846 to 898 and 1019 to 1024. In some embodiments, the dsRNA includes a sense strand having 20 contiguous nucleotides differing by no more than 2 nucleotides from the nucleotide sequence selected from SEQ ID NOs: 793 to 845 and 1013 to 1018. In some embodiments, the dsRNA includes an antisense strand having 20 contiguous nucleotides differing by no more than 2 nucleotides from the nucleotide sequence selected from SEQ ID NOs: 846 to 898 and 1019 to 1024. In some embodiments, the dsRNA includes a sense strand having 21 contiguous nucleotides differing by no more than 2 nucleotides from the nucleotide sequence selected from SEQ ID NOs: 793 to 845 and 1013 to 1018. In some embodiments, the dsRNA includes an antisense strand having 21 contiguous nucleotides differing by no more than 2 nucleotides from the nucleotide sequence selected from SEQ ID NOs: 846 to 898 and 1019 to 1024.

In some embodiments, the dsRNA includes a sense strand having 10 contiguous nucleotides differing by no more than 1 nucleotide from the nucleotide sequence selected from SEQ ID NOs: 793 to 845 and 1013 to 1018. In some embodiments, the dsRNA includes an antisense strand having 10 contiguous nucleotides differing by no more than 1 nucleotide from the nucleotide sequence selected from SEQ ID NOs: 846 to 898 and 1019 to 1024. In some embodiments, the dsRNA includes a sense strand having 11 contiguous nucleotides differing by no more than 1 nucleotide from the nucleotide sequence selected from SEQ ID NOs: 793 to 845 and 1013 to 1018. In some embodiments, the dsRNA includes an antisense strand having 11 contiguous nucleotides differing by no more than 1 nucleotide from the nucleotide sequence selected from SEQ ID NOs: 846 to 898 and 1019 to 1024. In some embodiments, the dsRNA includes a sense strand having 12 contiguous nucleotides differing by no more than 1 nucleotide from the nucleotide sequence selected from SEQ ID NOs: 793 to 845 and 1013 to 1018. In some embodiments, the dsRNA includes an antisense strand having 12 contiguous nucleotides differing by no more than 1 nucleotide from the nucleotide sequence selected from SEQ ID NOs: 846 to 898 and 1019 to 1024. In some embodiments, the dsRNA includes a sense strand having 13 contiguous nucleotides differing by no more than 1 nucleotide from the nucleotide sequence selected from SEQ ID NOs: 793 to 845 and 1013 to 1018. In some embodiments, the dsRNA includes an antisense strand having 13 contiguous nucleotides differing by no more than 1 nucleotide from the nucleotide sequence selected from SEQ ID NOs: 846 to 898 and 1019 to 1024. In some embodiments, the dsRNA includes a sense strand having 14 contiguous nucleotides differing by no more than 1 nucleotide from the nucleotide sequence selected from SEQ ID NOs: 793 to 845 and 1013 to 1018. In some embodiments, the dsRNA includes an antisense strand having 14 contiguous nucleotides differing by no more than 1 nucleotide from the nucleotide sequence selected from SEQ ID NOs: 846 to 898 and 1019 to 1024. In some embodiments, the dsRNA includes a sense strand having 15 contiguous nucleotides differing by no more than 1 nucleotide from the nucleotide sequence selected from SEQ ID NOs: 793 to 845 and 1013 to 1018. In some embodiments, the dsRNA includes an antisense strand having 15 contiguous nucleotides differing by no more than 1 nucleotide from the nucleotide sequence selected from SEQ ID NOs: 846 to 898 and 1019 to 1024. In some embodiments, the dsRNA includes a sense strand having 16 contiguous nucleotides differing by no more than 1 nucleotide from the nucleotide sequence selected from SEQ ID NOs: 793 to 845 and 1013 to 1018. In some embodiments, the dsRNA includes an antisense strand having 16 contiguous nucleotides differing by no more than 1 nucleotide from the nucleotide sequence selected from SEQ ID NOs: 846 to 898 and 1019 to 1024. In some embodiments, the dsRNA includes a sense strand having 17 contiguous nucleotides differing by no more than 1 nucleotide from the nucleotide sequence selected from SEQ ID NOs: 793 to 845 and 1013 to 1018. In some embodiments, the dsRNA includes an antisense strand having 17 contiguous nucleotides differing by no more than 1 nucleotide from the nucleotide sequence selected from SEQ ID NOs: 846 to 898 and 1019 to 1024. In some embodiments, the dsRNA includes a sense strand having 18 contiguous nucleotides differing by no more than 1 nucleotide from the nucleotide sequence selected from SEQ ID NOs: 793 to 845 and 1013 to 1018. In some embodiments, the dsRNA includes an antisense strand having 18 contiguous nucleotides differing by no more than 1 nucleotide from the nucleotide sequence selected from SEQ ID NOs: 846 to 898 and 1019 to 1024. In some embodiments, the dsRNA includes a sense strand having 19 contiguous nucleotides differing by no more than 1 nucleotide from the nucleotide sequence selected from SEQ ID NOs: 793 to 845 and 1013 to 1018. In some embodiments, the dsRNA includes an antisense strand having 19 contiguous nucleotides differing by no more than 1 nucleotide from the nucleotide sequence selected from SEQ ID NOs: 846 to 898 and 1019 to 1024. In some embodiments, the dsRNA includes a sense strand having 20 contiguous nucleotides differing by no more than 1 nucleotide from the nucleotide sequence selected from SEQ ID NOs: 793 to 845 and 1013 to 1018. In some embodiments, the dsRNA includes an antisense strand having 20 contiguous nucleotides differing by no more than 1 nucleotide from the nucleotide sequence selected from SEQ ID NOs: 846 to 898 and 1019 to 1024. In some embodiments, the dsRNA includes a sense strand having 21 contiguous nucleotides differing by no more than 1 nucleotide from the nucleotide sequence selected from SEQ ID NOs: 793 to 845 and 1013 to 1018. In some embodiments, the dsRNA includes an antisense strand having 21 contiguous nucleotides differing by no more than 1 nucleotide from the nucleotide sequence selected from SEQ ID NOs: 846 to 898 and 1019 to 1024. In some embodiments, the dsRNA includes an antisense strand having 22 contiguous nucleotides differing by no more than 1 nucleotide from the nucleotide sequence selected from SEQ ID NOs: 846 to 898 and 1019 to 1024. In some embodiments, the dsRNA includes an antisense strand having 23 contiguous nucleotides differing by no more than 1 nucleotide from the nucleotide sequence selected from SEQ ID NOs: 846 to 898 and 1019 to 1024.

In some embodiments, the dsRNA includes (i) a sense strand having 15 contiguous nucleotides differing by no more than one, two or three nucleotides from the nucleotide sequence selected from SEQ ID NOs: 793 to 845 and 1013 to 1018 and (ii) an antisense strand forming a duplex with the sense strand of (i) and having 15 contiguous nucleotides differing by no more than one, two or three nucleotides from the nucleotide sequence selected from SEQ ID NOs: 846 to 898 and 1019 to 1024. In some embodiments, the dsRNA includes (i) a sense strand having 16 contiguous nucleotides differing by no more than one, two or three from the nucleotide sequence selected from SEQ ID NOs: 793 to 845 and 1013 to 1018 and (ii) an antisense strand forming a duplex with the sense strand of (i) and having 16 contiguous nucleotides differing by no more than one, two or three nucleotides from the nucleotide sequence selected from SEQ ID NOs: 846 to 898 and 1019 to 1024. In some embodiments, the dsRNA includes (i) a sense strand having 17 contiguous nucleotides differing by no more than one, two or three nucleotides from the nucleotide sequence selected from SEQ ID NOs: 793 to 845 and 1013 to 1018 and (ii) an antisense strand forming a duplex with the sense strand of (i) and having 17 contiguous nucleotides differing by no more than one, two or three nucleotides from the nucleotide sequence selected from SEQ ID NOs: 846 to 898 and 1019 to 1024. In some embodiments, the dsRNA includes (i) a sense strand having 18 contiguous nucleotides differing by no more than one, two or three nucleotides from the nucleotide sequence selected from SEQ ID NOs: 793 to 845 and 1013 to 1018 and (ii) an antisense strand forming a duplex with the sense strand of (i) and having 18 contiguous nucleotides differing by no more than one, two or three nucleotides from the nucleotide sequence selected from SEQ ID NOs: 846 to 898 and 1019 to 1024. In some embodiments, the dsRNA includes (i) a sense strand having 19 contiguous nucleotides differing by no more than one, two or three nucleotides from the nucleotide sequence selected from SEQ ID NOs: 793 to 845 and 1013 to 1018 and (ii) an antisense strand forming a duplex with the sense strand of (i) and having 19 contiguous nucleotides differing by no more than one, two or three nucleotides from the nucleotide sequence selected from SEQ ID NOs: 846 to 898 and 1019 to 1024. In some embodiments, the dsRNA includes (i) a sense strand having 20 contiguous nucleotides differing by no more than one, two or three nucleotides from the nucleotide sequence selected from SEQ ID NOs: 793 to 845 and 1013 to 1018 and (ii) an antisense strand forming a duplex with the sense strand of (i) and having 20 contiguous nucleotides differing by no more than one, two or three nucleotides from the nucleotide sequence selected from SEQ ID NOs: 846 to 898 and 1019 to 1024. In some embodiments, the dsRNA includes (i) a sense strand having 21 contiguous nucleotides differing by no more than one, two or three nucleotides from the nucleotide sequence selected from SEQ ID NOs: 793 to 845 and 1013 to 1018 and (ii) an antisense strand forming a duplex with the sense strand of (i) and having 21 contiguous nucleotides differing by no more than one, two or three nucleotides from the nucleotide sequence selected from SEQ ID NOs: 846 to 898 and 1019 to 1024.

In certain aspects, when a sense strand or an antisense strand of a dsRNA in above paragraphs is differing by a certain number of nucleotides (e.g., one, two or three nucleotides) from a specific sequence (e.g., SEQ ID NOs: 793-898 and 1013-1024), it is meant by that the sense strand or the antisense strand includes one, two or three nucleotides, having different nucleobases and/or different modifications compared to the nucleobases and/or the modifications of the nucleotides at the corresponding positions of the specific sequence (e.g., SEQ ID NOs: 793-898 and 1013-1024). In some embodiments, when a sense strand or an antisense strand is differing by a certain number of nucleotides (e.g., one, two or three nucleotides) from a specific sequence (e.g., SEQ ID NOs: 793-898 and 1013-1024), the sense strand or the antisense strand includes one, two, or three nucleotides, having different nucleobases compared to the nucleobases of the nucleotides at the corresponding positions of the specific sequence (e.g., SEQ ID NOs: 793-898 and 1013-1024). In some embodiments, when a sense strand or an antisense strand is differing by a certain number of nucleotides (e.g., one, two or three nucleotides) from a specific sequence (e.g., SEQ ID NOs: 793-898 and 1013-1024), the sense strand or the antisense strand includes one, two, or three nucleotides, having different modifications compared to the modifications of the nucleotides at the corresponding positions of the specific sequence (e.g., SEQ ID NOs: 793-898 and 1013-1024). In some embodiments, when a sense strand or an antisense strand is differing by a certain number of nucleotides (e.g., one, two or three nucleotides) from a specific sequence (e.g., SEQ ID NOs: 793-898 and 1013-1024), the sense strand or the antisense strand includes one, two, or three nucleotides having different nucleobases and different modifications compared to the nucleobases and the modifications of the nucleotides at the corresponding positions of the specific sequence (e.g., SEQ ID NOs: 793-898 and 1013-1024).

In certain aspects, when a sense strand or an antisense strand of a dsRNA in above paragraphs is differing by a certain number of nucleotides (e.g., one, two or three nucleotides) from a specific sequence (e.g., SEQ ID NOs: 793-898 and 1013-1024), it is meant by that the sense strand or the antisense strand includes one, two or three nucleotides, having different nucleobases, different modifications, and/or different phosphate linkages (e.g., phosphorothioate (PS)), compared to the nucleobases, the modifications, and/or the phosphate linkages of the nucleotides at the corresponding positions of the specific sequence (e.g., SEQ ID NOs: 793-898 and 1013-1024). In some embodiments, when a sense strand or an antisense strand is differing by a certain number of nucleotides (e.g., one, two or three nucleotides) from a specific sequence (e.g., SEQ ID NOs: 793-898 and 1013-1024), the sense strand or the antisense strand includes one, two, or three nucleotides, having different phosphate linkages compared to the phosphate linkages of the nucleotides at the corresponding positions of the specific sequence (e.g., SEQ ID NOs: 793-898 and 1013-1024). In some embodiments, when a sense strand or an antisense strand is differing by a certain number of nucleotides (e.g., one, two or three nucleotides) from a specific sequence (e.g., SEQ ID NOs: 793-898 and 1013-1024), the sense strand or the antisense strand includes one, two, or three nucleotides, having different nucleobases and different phosphate linkages compared to the nucleobases and the phosphate linkages of the nucleotides at the corresponding positions of the specific sequence (e.g., SEQ ID NOs: 793-898 and 1013-1024). In some embodiments, when a sense strand or an antisense strand is differing by a certain number of nucleotides (e.g., one, two or three nucleotides) from a specific sequence (e.g., SEQ ID NOs: 793-898 and 1013-1024), the sense strand or the antisense strand includes one, two, or three nucleotides, having different modifications and different phosphate linkages compared to the modifications and the phosphate linkages of the nucleotides at the corresponding positions of the specific sequence (e.g., SEQ ID NOs: 793-898 and 1013-1024). In some embodiments, when a sense strand or an antisense strand is differing by a certain number of nucleotides (e.g., one, two or three nucleotides) from a specific sequence (e.g., SEQ ID NOs: 793-898 and 1013-1024), the sense strand or the antisense strand includes one, two, or three nucleotides having different nucleobases, different modifications, and different phosphate linkages compared to the nucleobases, the modifications, and the phosphate linkages of the nucleotides at the corresponding positions of the specific sequence (e.g., SEQ ID NOs: 793-898 and 1013-1024).

In some embodiments, the dsRNA includes a sense strand comprising SEQ ID NO: 800, and an antisense strand comprising SEQ ID NO: 853.

In some embodiments, the dsRNA includes a sense strand comprising SEQ ID NO: 801, and an antisense strand comprising SEQ ID NO: 854.

In some embodiments, the dsRNA includes a sense strand comprising SEQ ID NO: 806, and an antisense strand comprising SEQ ID NO: 859.

In some embodiments, the dsRNA includes a sense strand comprising SEQ ID NO: 811, and an antisense strand comprising SEQ ID NO: 864.

In some embodiments, the dsRNA includes a sense strand comprising SEQ ID NO: 813, and an antisense strand comprising SEQ ID NO: 866.

In some embodiments, the dsRNA includes a sense strand comprising SEQ ID NO: 830, and an antisense strand comprising SEQ ID NO: 883.

### Ligands

In an aspect, a ligand including the various chemical and biological moieties, such as a small molecule compound, a peptide, an antibody, a carbohydrate, or an additional nucleic acid with or without a linker, can be coupled or conjugated to a dsRNA as described herein. In certain aspects, the ligand may directly (e.g., covalently) conjugated to at least one strand of the dsRNA.

In certain aspects, the ligand may be conjugated via a linker thereof (e.g., covalent linker) to a sense strand. In some embodiments, the ligand may be conjugated via a linker (e.g., covalent linker, or phosphate or phosphorothioate group) to one or more nucleotides in the sense strand. In some embodiments, the ligand may be conjugated via a linker (e.g., covalent linker, or phosphate or phosphorothioate group) to 5′ end of the sense strand. In some embodiments, the ligand may be conjugated via a linker (e.g., covalent linker, or phosphate or phosphorothioate group) to 3′ end of the sense strand.

In certain aspects, the ligand may be conjugated via a linker (e.g., covalent linker) to an antisense strand. In some embodiments, the ligand may be conjugated via a linker (e.g., covalent linker, or phosphate or phosphorothioate group) to one or more nucleotides of an antisense strand. In some embodiments, the ligand may be conjugated via a linker (e.g., covalent linker, or phosphate or phosphorothioate group) to 5′ end of an antisense strand. In some embodiments, the ligand may be conjugated via a linker (e.g., covalent linker, or phosphate or phosphorothioate group) to 3′ end of an antisense strand.

In certain aspects, the linker may be a cleavable chemical moiety which is sufficiently stable outside the cell but which upon is spontaneously and/or irreversibly cleaved to release one or more conjugated groups (e.g., targeting moiety) when introduced in a cell or other physiological conditions (e.g., serum, or blood). In some embodiments, the cleavable linker may include a cleavage site at its terminal part that is attached to other compounds or molecules. In some embodiments, the cleavable linker may include a cleavage site that locates between the two-terminus attached to each different compound or molecule.

In certain aspects, the linker may be a non-cleavable linker. In certain aspects, the linker may be a hydrolysable linker.

A choice of the ligand may provide an enhanced affinity and/or delivery of the dsRNA to a specific target biomolecule, cell, tissue, organ compartment, or organ or region of a body. In certain aspects, the ligand may include a targeting moiety or group which bind to a specific organ cell, e.g., liver or kidney cell. In some embodiments, the ligand may include a targeting moiety or group which bind to a specific cell type, e.g., a cancer cell, endothelial cell, or bone cell. In certain aspects, the ligand may include a targeting moiety to hormones and hormone receptors. In certain aspects, the ligand may include a targeting moiety including a lipid component (e.g., short/long chain fatty acid, cationic lipid, lipophilic molecule, cholesterol, steroid, uvaol, hecigenin, diosgenin, terpene, triterpene, sarsasapogenin, friedelin, epifriedelanol-derivatized lithocholic acid, etc.) to modulate or control the binding, to increase resistance to degradation, or to increase targeting or transport into a target cell membrane or cellular lipid vesicles.

Non limiting examples of ligands may include, but not be limited to, proteins (e.g., thyrotropin, melanotropin, lectin, glycoprotein such as transferrin, or surfactant protein A), carbohydrates (e.g., mucin carbohydrate, multivalent lactose, multivalent galactose, N-acetyl-galactosamine, N-acetyl-glucosamine, multivalent mannose, multivalent fucose, glycosylated polyaminoacids, or multivalent galactose), small molecule drugs (e.g., bisphosphonate), polymers (e.g., PEG (e.g., PEG-40K), MPEG, [MPEG]2, polyglutamate, or polyaspartate), a lipid component (e.g., cholesterol, a steroid, bile acid, cholic acid, adamantane acetic acid, 1-pyrene butyric acid, dihydrotestosterone, 1,3-bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, or 03-(oleoyl)cholenic acid), organic compounds (e.g., dimethoxytrityl, or phenoxazine), vitamins (e.g., folate, vitamin B12, or biotin), small peptides (e.g., antennapedia peptide, TAT peptide, RGD peptide, an RGD peptide mimetic), an additional nucleic acids (e.g., an aptamer), dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases or a chelator (e.g., EDTA), radiolabeled markers, enzymes, or the like.

In some embodiments, the ligand may include carbohydrates (e.g., mucin carbohydrate, multivalent lactose, multivalent galactose, N-acetyl-galactosamine, N-acetyl-glucosamine multivalent mannose, multivalent fucose, glycosylated polyaminoacids, or multivalent galactose) as the targeting moiety. In some embodiments, the ligand may include multivalent lactose or multivalent galactose. In some embodiments, the ligand may include N-acetyl-galactosamine as the targeting moiety.

In certain aspects, the ligand may include one or more diagnostic compound, reporter group, cross-linking agent, nuclease-resistance conferring moiety, modified or unmodified nucleobase, lipophilic molecule, cholesterol, lipid, lectin, steroid, uvaol, hecigenin, diosgenin, terpene, triterpene, sarsasapogenin, friedelin, epifriedelanol-derivatized lithocholic acid, vitamin, carbohydrate, dextran, pullulan, chitin, chitosan, synthetic carbohydrate, oligo lactate (e.g., 15-mer), natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

In certain aspects, the ligand targets a specific receptor on a cell (e.g., liver cell or kidney cell). In some embodiments, the ligand targets a cell surface protein, e.g., asialoglycoprotein receptor (ASGPR), which is abundantly expressed on liver cells (hepatocytes). In some embodiments, for targeting ASGPR, the ligand may include one or more selected from carbohydrate (e.g., pyranose such as glucose or its derivatives (e.g., GluNAc), galactose or its derivatives (e.g., GalNAc), mannose or its derivatives (e.g., mannose-6P)). In some embodiments, the ligand may include a sugar cluster containing two or more sugar moieties (e.g., glucose or its derivatives, galactose or its derivatives (e.g., GalNAc), mannose or its derivatives (e.g., m annose-6P), and etc.). In some embodiments, the ligand may include galactose cluster, e.g., GalNAc cluster, or mannose cluster. In some embodiments, the cluster may be formed by linking or coupling the sugar moieties via one or more covalent linkers.

In certain aspects, the ligand includes one or more GalNAc moieties. In some embodiments, the ligand includes one GalNAc moiety. In some embodiments, the ligand includes two GalNAc moieties. In some embodiments, the ligand includes three GalNAc moieties. In some embodiments, the ligand may include one or more covalent linkers.

In certain aspects, the ligand has a structure of Formula (A):

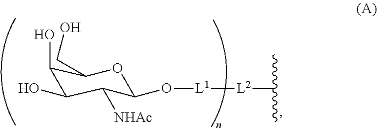

or a pharmaceutically acceptable salt thereof,
wherein:
each L¹ is an independently a linker which may be same or different in each occurrence;
L² is a linker;
n is an integer from 1 to 3; and
[symbol] is an attachment point to the sense strand or the antisense strand, or to a conjugate linker conjugated to the sense strand or the antisense strand.

In some embodiments, the attachment point is connected to the sense strand or the antisense strand via a direct bond. In some embodiments, the attachment point is connected to the sense strand and the antisense strand via “a conjugate linker” that connects the ligand and one or both strands of dsRNA (e.g., sense strand or antisense strand). In some embodiments, the attachment point is connected to the sense strand and the antisense strand via the conjugate linker that may form a phosphodiester linkage. In some embodiments, the attachment point is connected to the sense strand and the antisense strand via a conjugate linker that may form a phosphorothioate linkage.

In some embodiments, the attachment point is connected to the sense strand via a phosphodiester linkage at the 3′ end of the sense strand. In some embodiments, the attachment point is connected to the sense strand via a phosphodiester linkage at the 5′ end of the sense strand. In some embodiments, the attachment point is connected to the sense strand via a phosphorothioate group at the 3′ end of the sense strand. In some embodiments, the attachment point is connected to the sense strand via a phosphorothioate group at the 5′ end of the sense strand.

In some embodiments, the attachment point is connected to the antisense strand via a phosphodiester linkage at the 3′ end of the antisense strand. In some embodiments, the attachment point is connected to the antisense strand via a phosphodiester linkage at the 5′ end of the antisense strand. In some embodiments, the attachment point is connected to the antisense strand via a phosphorothioate group at the 3′ end of the antisense strand. In some embodiments, the attachment point is connected to the antisense strand via a phosphorothioate group at the 5′ end of the antisense strand.

In certain aspects, each L¹ is independently a covalent linker having the formula -L^{1A}-L^{1B}-L^{1C}-L^{1D}-L^{1E}-. Each L^{1A}, L^{1B}, L^{1C}, L^{1D}, and L^{1E} is independently a bond, —O—P(S)(O)—O—, or —O—P(O)(O)—O—, —O—P(S)(O)—, or —O—P(O)(O)—, a substituted or unsubstituted alkylene (e.g., C₁-C₃₀ alkylene, C₁-C₂₅ alkylene, C₁-C₁₂ or C₁-C₈ alkylene), a substituted or unsubstituted heteroalkylene (e.g., 2 to 30 membered heteroalkylene, 2 to 15 membered heteroalkylene, 2 to 12 membered heteroalkylene, or 2 to 8 membered heteroalkylene), a substituted or unsubstituted cycloalkylene (e.g., C₄-C₁₂ cycloalkylene), a substituted or unsubstituted heterocycloalkylene (e.g., 2 to 30 membered, 2 to 15 membered, or 2 to 12 membered heteroalkylene heterocycloalkylene), substituted or unsubstituted arylene (e.g., C₆-C₁₂ arylene), or a substituted or unsubstituted heteroarylene (e.g., 5 to 6 membered heteroarylene). In some embodiments, each L^{1A}, L^{1B}, L^{1C}, L^{1D}, and L^{1E} is independently a bond, a substituted or unsubstituted alkylene (e.g., C₁-C₃₀, C₁-C₁₅, or C₁-C₁₂ alkylene), a substituted or unsubstituted heteroalkylene (e.g., 2 to 12 membered heteroalkylene), substituted or unsubstituted arylene (e.g., phenylene), or substituted or unsubstituted heteroarylene (e.g., pyridylene). In some embodiments, each L^{1A}, L^{1B}, L^{1C}, L^{1D}, and L^{1E} is independently a bond, unsubstituted C₁-C₁₂ alkylene, —NHC(O)—, —C(O)NH—, —(CH₂)ₐ₁—O—, —O—(CH₂)ₐ₁—, —(CH₂CH₂O)_{b1}—, —(OCH₂CH₂)_{b1}—, —O—P(S)(O)—O—, or —O—P(O)(O)—O—, and each a1 or b1 is independently an integer from 0 to 12.

In some embodiments, L¹ has the following structure:

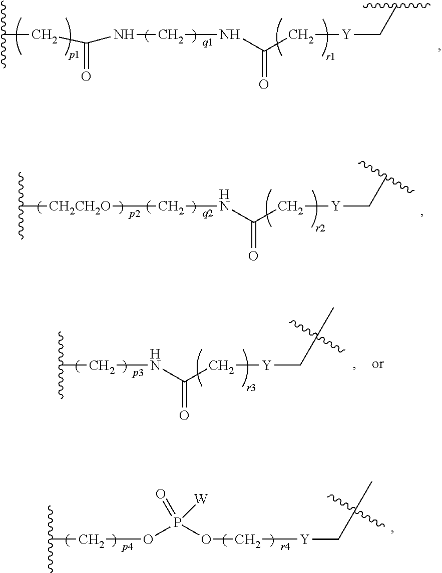

or a pharmaceutically acceptable salt thereof,
wherein:
each p1, p2, p3, p4, q1, q2, r1, r2, r3 and r4 is independently an integer from 0 to 12;
W is —OH or —SH; and
Y is —O— or absent.

In some embodiments, W is —OH. In some embodiments, W is —SH.

In some embodiments, Y is —O—. In some embodiments, Y is absent.

In some embodiments, L¹ has the following structure:

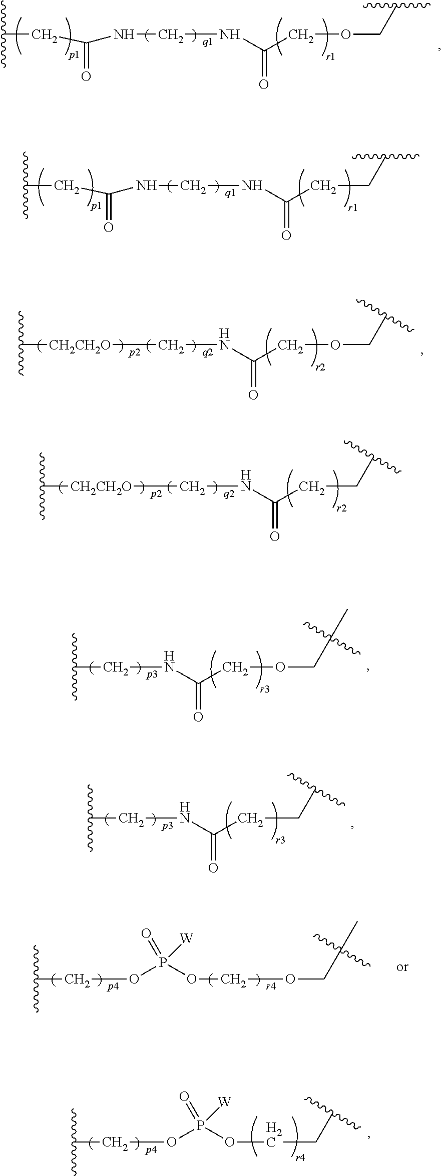

or a pharmaceutically acceptable salt thereof.
p1, p2, p3, p4, q1, q2, r1, r2, r3, r4, and W are as described above.

In some embodiments, each p1, p2, p3, p4, q1, q2, r1, r2, r3 and r4 is independently an integer from 1 to 6. In some embodiments, each p1, p2, p3, and p4 is independently 2, 3, 4, 5, 6, or 8. In some embodiments, each q1 and q2 is independently 1, 2, 3, or 4. In some embodiments, each r1, r2, r3 and r4 is independently 1, 2, 3, or 4.

In certain aspects, the ligand includes the following structures (e.g., GalNAc moiety):

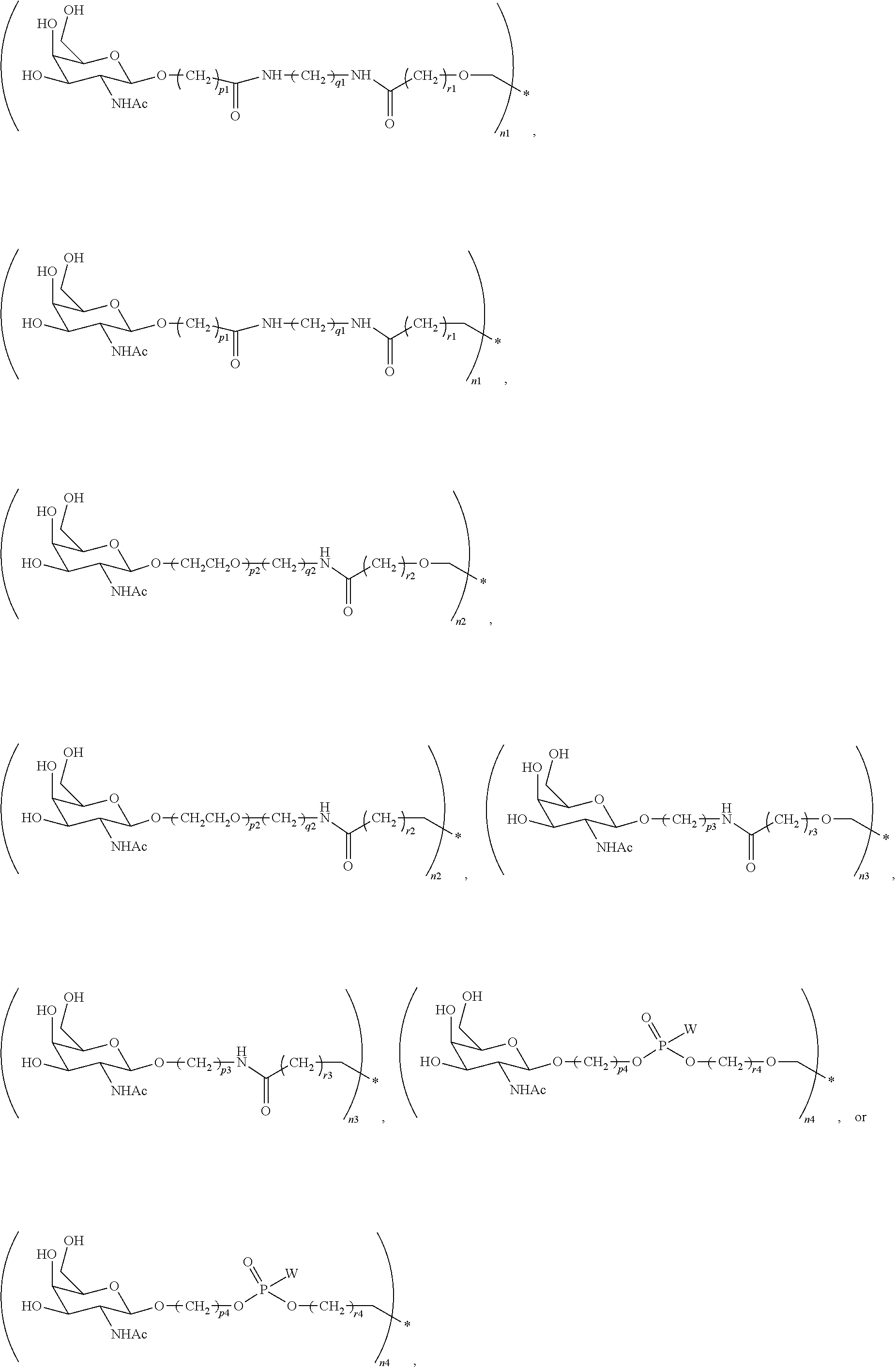

or a pharmaceutically acceptable salt thereof,
wherein each n1, n2, n3, and n4 is independently an integer from 1 to 3. Y, W, p1, p2, p3, p4, q1, q2, r1, r2, r3, and r4 are as described above, and “*” is an attachment point to L² in Formula (A).

In some embodiments, n1 is 1. In some embodiments, n1 is 2. In some embodiments, n1 is 3. In some embodiments, n2 is 1. In some embodiments, n2 is 2. In some embodiments, n2 is 3. In some embodiments, n3 is 1. In some embodiments, n3 is 2. In some embodiments, n3 is 3. In some embodiments, n4 is 1. In some embodiments, n4 is 2. In some embodiments, n4 is 3.

In some embodiments, the ligand includes the following structure:

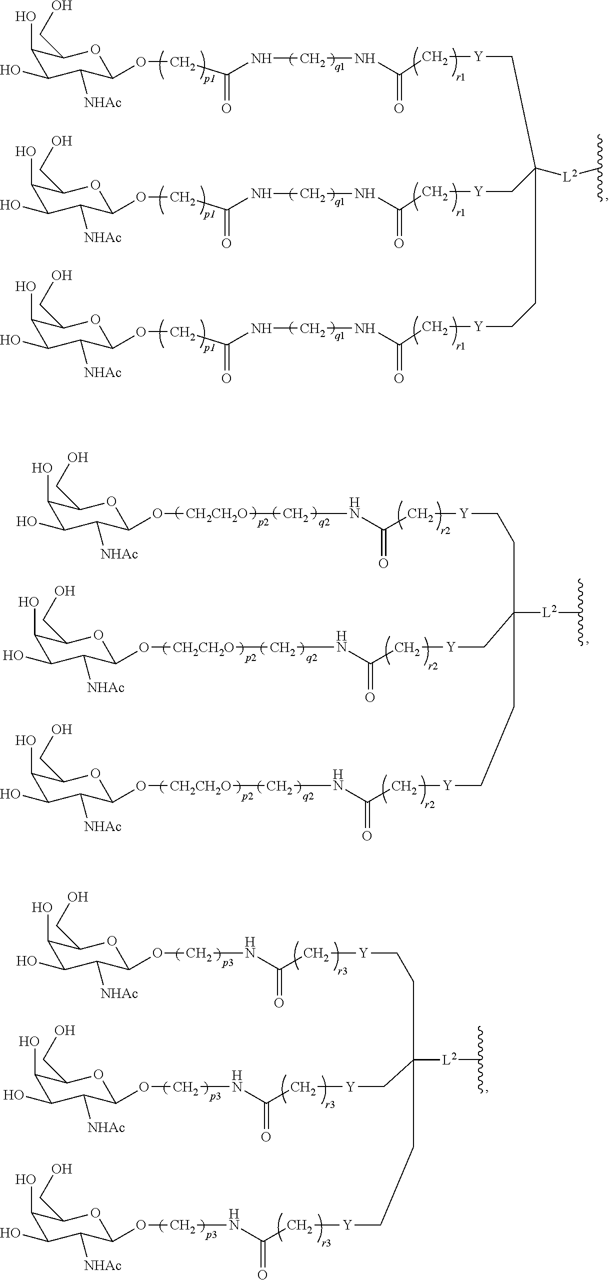

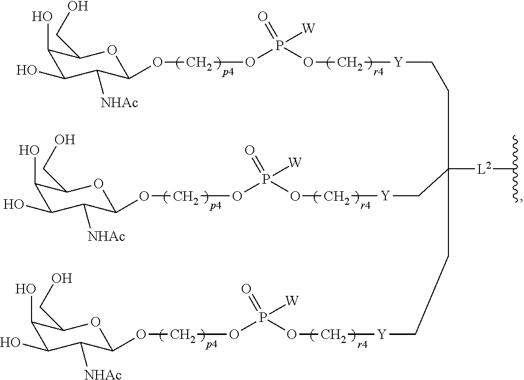

or a pharmaceutically acceptable salt thereof,
wherein L², Y, W, p1, p2, p3, p4, q1, q2, r1, r2, r3, and r4 are as described above and
[symbol] is an attachment point to the sense strand or the antisense strand, or to a conjugate linker conjugated to the sense strand or the antisense strand.

In certain aspects, L² is a covalent linker of the formula -L^{2A}-L^{2B}-L^{2C}-L^{2D}-L^{2E}-. Each L^{2A}, L^{2B}, L^{2C}, L^{2D}, and L^{2E} a bond, —O—P(S)(O)—O—, —O—P(O)(O)—O—, —O—P(S)(O)—, or —O—P(O)(O)—, a substituted or unsubstituted alkylene (e.g., C₁-C₃₀ alkylene, C₁-C₂₅ alkylene, C₁-C₁₂ or C₁-C₈ alkylene), a substituted or unsubstituted heteroalkylene (e.g., 2 to 30 membered heteroalkylene, 2 to 15 membered heteroalkylene, 2 to 12 membered heteroalkylene, or 2 to 8 membered heteroalkylene), a substituted or unsubstituted cycloalkylene (e.g., C₄-C₁₂ cycloalkylene), a substituted or unsubstituted heterocycloalkylene (e.g., 5 to 6 membered heterocycloalkylene), substituted or unsubstituted arylene (e.g., phenylene), or a substituted or unsubstituted heteroarylene (e.g., 5 to 6 membered heteroarylene). In some embodiments, each L^{2A}, L^{2B}, L^{2C}, L^{2D}, and L^{2E} is independently a bond, substituted or unsubstituted alkylene (e.g., C₁-C₃₀ alkylene, C₁-C₂₅ alkylene, C₁-C₁₂ or C₁-C₈ alkylene), a substituted or unsubstituted heteroalkylene (e.g., 2 to 30 membered, 2 to 15 membered, or 2 to 12 membered heteroalkylene heteroalkylene), or a substituted or unsubstituted heterocycloalkylene (e.g., 5 to 6 membered heterocycloalkylene). In some embodiments, each L^{2A}, L^{2B}, L^{2C}, L^{2D}, and L^{2E} is independently a bond, substituted (e.g., OH-substituted) or unsubstituted C₁-C₁₂ alkylene, —NHC(O)—, —C(O)NH—, —(CH₂)ₐ₂—O—, —O—(CH₂)ₐ₂—, —(CH₂CH₂O)_{b2}—, —(OCH₂CH₂)_{b2}—, —O—P(S)(O)—O—, —O—P(O)(O)—O—, —O—P(S)(O)—, or —O—P(O)(O)—, substituted or unsubstituted cycloalkylene (e.g., cyclohexylene), substituted or unsubstituted heterocycloalkylene (e.g., pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin) or substituted or unsubstituted arylene (e.g., phenylene). Each a2 or b2 is independently an integer from 1 to 12.

In some embodiments, L^{2A} is —NHC(O)—, or —C(O)NH—. In some embodiments, L^{2A} is —NHC(O)—.

In some embodiments, L^{2D} is

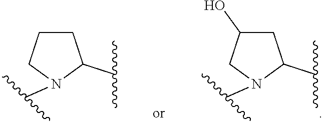 [SMILES: *C1CCCN1* ; *C1CC(O)CN1*]

In some embodiments, L^{2D} is

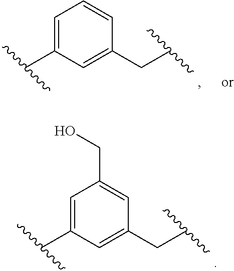 [SMILES: *Cc1cccc(*)c1 ; *Cc1cc(*)cc(CO)c1]

In some embodiments, L^{2D} is

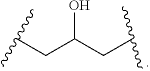 [SMILES: *CC(O)C*]

In some embodiments, L^{2D} is

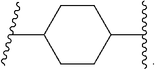 [SMILES: *C1CCC(*)CC1]

In some embodiments, L^{2D} is

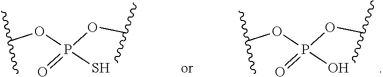 [SMILES: *OP(=O)(S)O* ; *OP(=O)(O)O*]

In some embodiments, L^{2D} is —O— or —S—.

In some embodiments, L^{2E} is a bond. In some embodiments, L^{2E} is —O— or —S—. In some embodiments, L^{2E} is

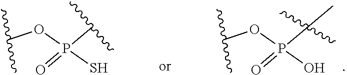 [SMILES: *OP(*)(=O)S ; *OP(*)(=O)O]

In some embodiments, L^{2E} is

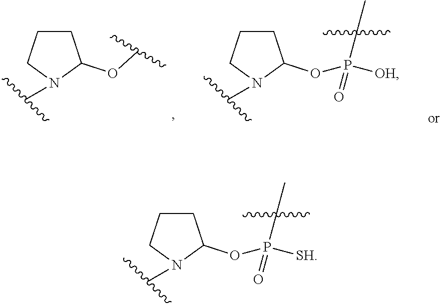 [SMILES: *OC1CCCN1* ; *N1CCCC1OP(*)(=O)O ; *N1CCCC1OP(*)(=O)S]

In some embodiments, L^{2E} is

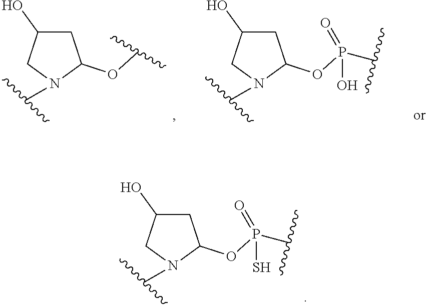 [SMILES: *OC1CC(O)CN1* ; *N1CC(O)CC1OP(*)(=O)O ; *N1CC(O)CC1OP(*)(=O)S]

In some embodiments, L^{2E} is

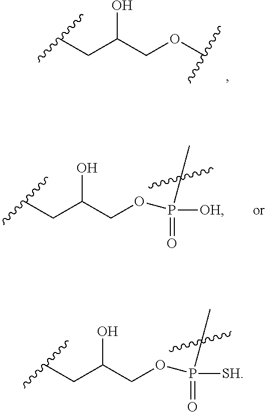 [SMILES: *CC(O)CO* ; *CC(O)COP(*)(=O)O ; *CC(O)COP(*)(=O)S]

In some embodiments, L^{2E} is

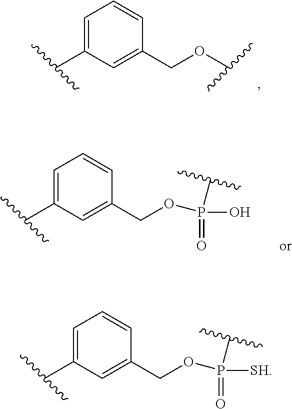 [SMILES: *OCc1cccc(*)c1 ; *c1cccc(COP(*)(=O)O)c1 ; *c1cccc(COP(*)(=O)S)c1]

In some embodiments,

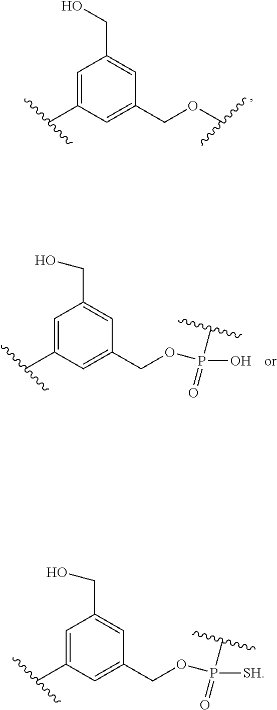 [SMILES: *OCc1cc(*)cc(CO)c1 ; *c1cc(CO)cc(COP(*)(=O)O)c1 ; *c1cc(CO)cc(COP(*)(=O)S)c1]

In some embodiments, L^{2E} is

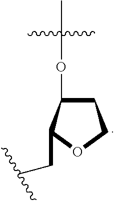

In some embodiments, the ligand includes the following structures (B-1) to (B-6):

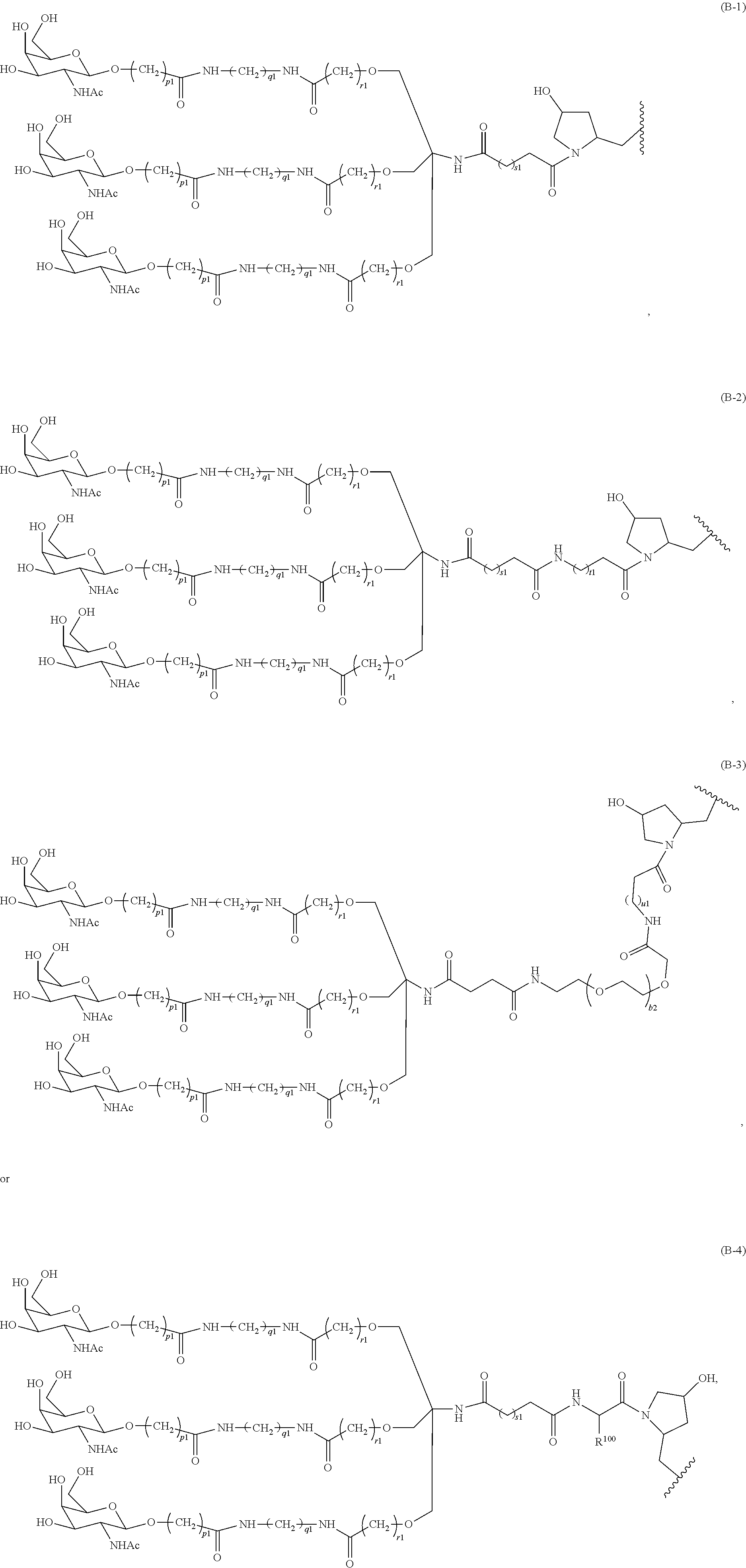

or a pharmaceutically acceptable salt thereof,
wherein each s1, t1, and u1 is independently an integer from 1 to 12, and R¹⁰⁰ is a hydrogen or a substituent (e.g., a side chain of a natural or unnatural amino acid). p1, q1, r1, and b2 are as described above.

Additional suitable ligands related to the above structures of Formula (B) and its subordinates and synthesis thereof are also described in WO2009/082607, entire contents of which are incorporated herein by reference.

In some embodiments, Y is —O—. In some embodiments, Y is absent.

In some embodiments, the ligand includes the following structures (C-1) to (C-3):

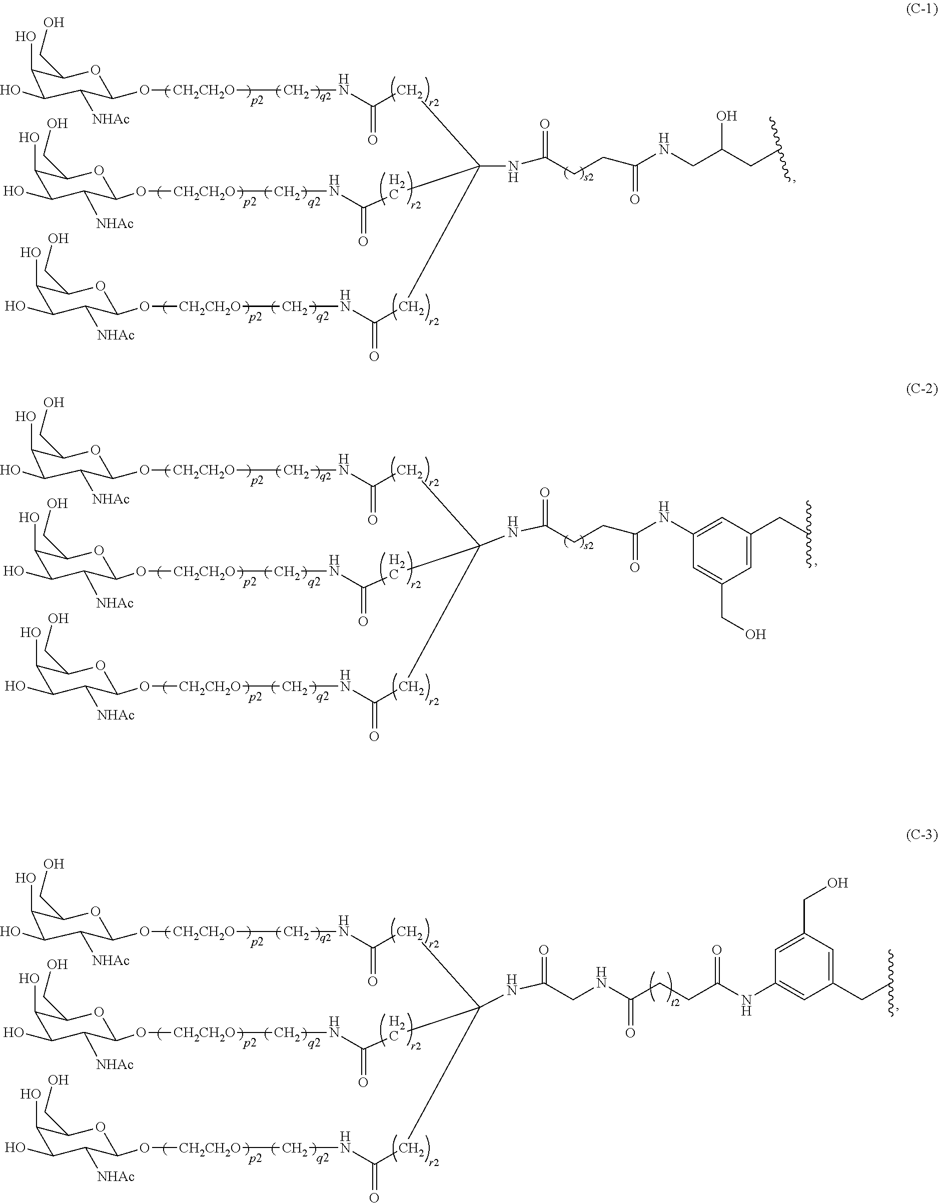

or a pharmaceutically acceptable salt thereof,
   wherein each s2 and t2 is independently an integer from 1 to 12. p2, q2, and r2 are as described above.

Additional suitable ligands related to the above structures of Formula (C) and its subordinates and synthesis thereof are also described in WO2018/191278, entire contents of which are incorporated herein by reference.

In some embodiments, the ligand includes the following structure (D):

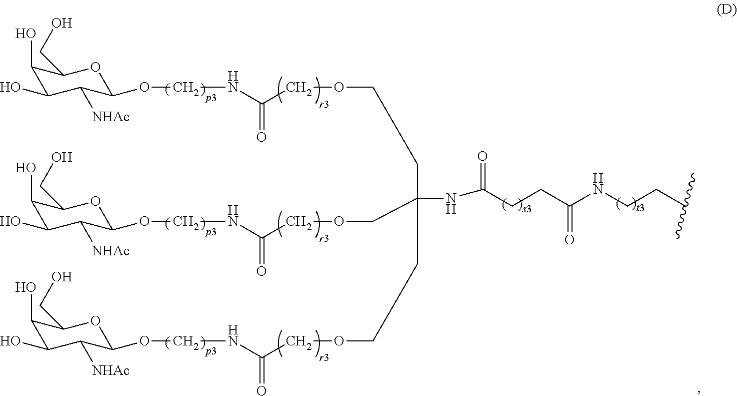

or a pharmaceutically acceptable salt thereof,
wherein each s3 and t3 is independently an integer from 1 to 12. p3 and r3 are as described above.

Additional suitable ligands related to the above structure of Formula (D) and its subordinates and synthesis thereof are also described in WO2014/179620, entire contents of which are incorporated herein by reference.

In some embodiments, the ligand includes the following structure (E-1) to (E-2):

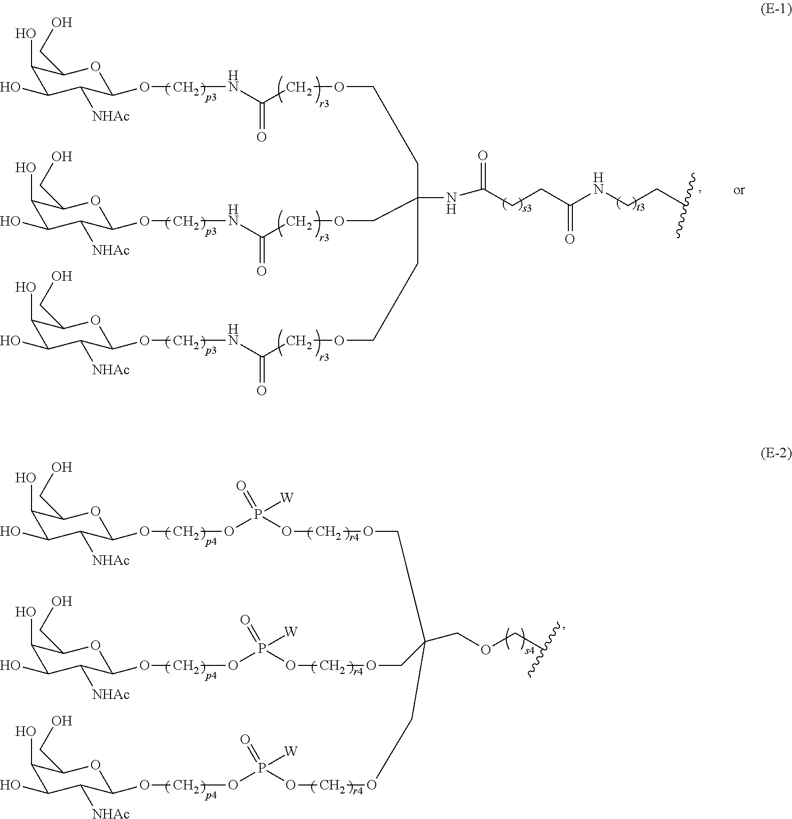

or a pharmaceutically acceptable salt thereof,
wherein each s4 is independently an integer from 1 to 12. W, p4, and r4 are as described above, a2 is 3 or 4, and [symbol] is an attachment point to the sense strand or the antisense strand, or to a conjugate linker conjugated to the sense strand or the antisense strand.

In some embodiments, at least one of W is —SH. In some embodiments, at least one of W is —OH.

Additional suitable ligands related to the above structure of Formula (E) and its subordinates and synthesis thereof are also described in WO2017/174657, entire contents of which are incorporated herein by reference.

In some embodiments, the ligand includes the following structures:

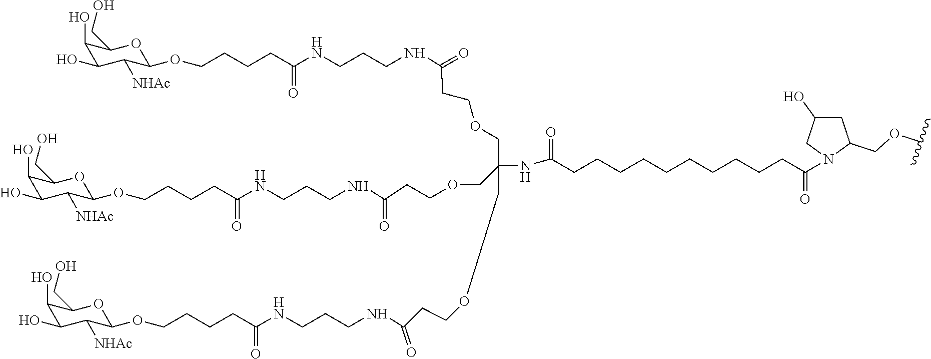 [SMILES: *OCC1CC(O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

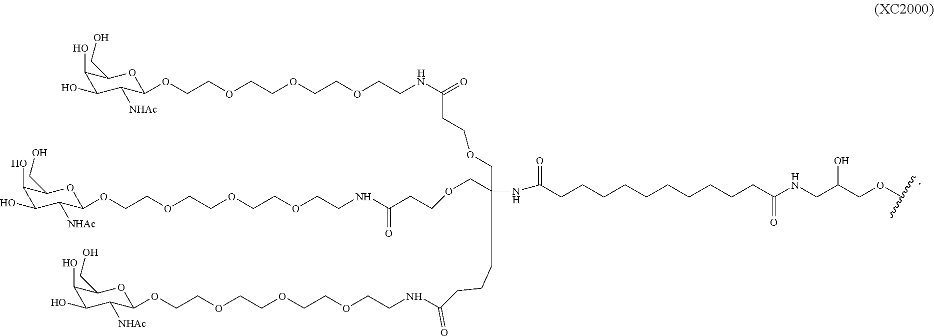 [SMILES: *OCC(O)CNC(=O)CCCCCCCCCCC(=O)NC(CCCC(=O)NCCOCCOCCOCCOC1OC(CO)C(O)C(O)C1NC(C)=O)(COCCC(=O)NCCOCCOCCOCCOC1OC(CO)C(O)C(O)C1NC(C)=O)COCCC(=O)NCCOCCOCCOCCOC1OC(CO)C(O)C(O)C1NC(C)=O]

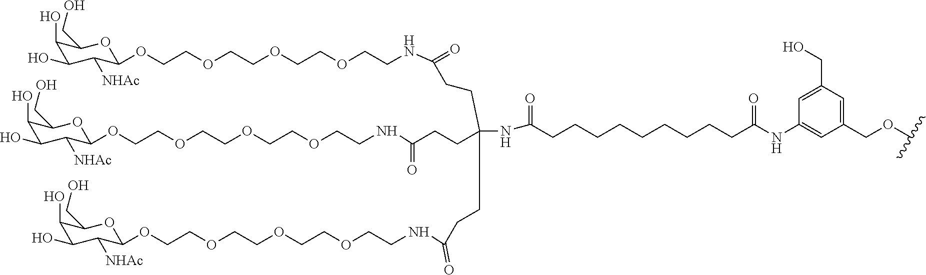 [SMILES: *OCc1cc(CO)cc(NC(=O)CCCCCCCCCC(=O)NC(CCC(=O)NCCOCCOCCOCCOC2OC(CO)C(O)C(O)C2NC(C)=O)(CCC(=O)NCCOCCOCCOCCOC2OC(CO)C(O)C(O)C2NC(C)=O)CCC(=O)NCCOCCOCCOCCOC2OC(CO)C(O)C(O)C2NC(C)=O)c1]

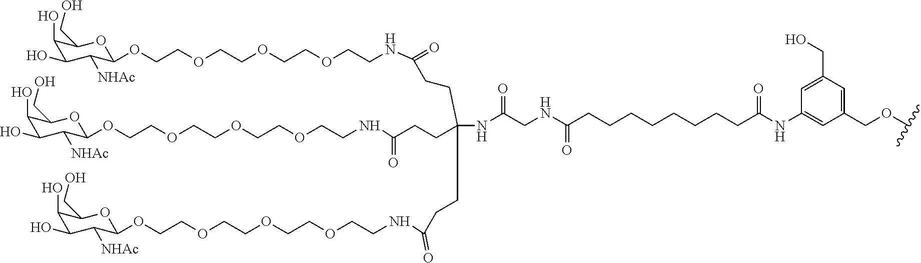 [SMILES: *OCc1cc(CO)cc(NC(=O)CCCCCCCCC(=O)NCC(=O)NC(CCC(=O)NCCOCCOCCOCCOC2OC(CO)C(O)C(O)C2NC(C)=O)(CCC(=O)NCCOCCOCCOCCOC2OC(CO)C(O)C(O)C2NC(C)=O)CCC(=O)NCCOCCOCCOCCOC2OC(CO)C(O)C(O)C2NC(C)=O)c1]

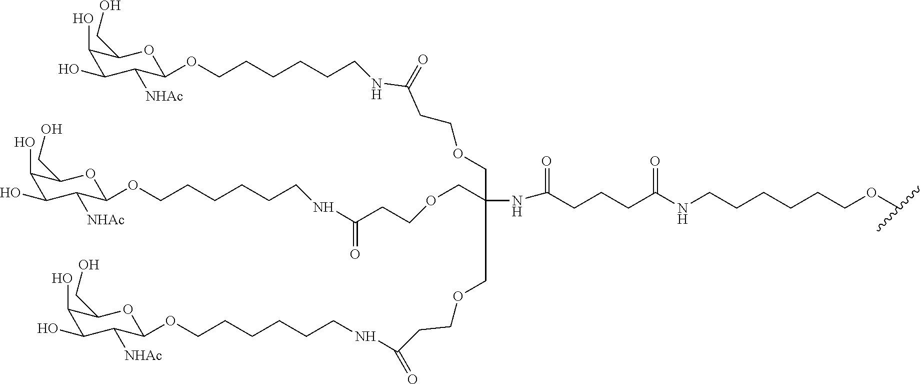 [SMILES: *OCCCCCCNC(=O)CCCC(=O)NC(COCCC(=O)NCCCCCCOC1OC(CO)C(O)C(O)C1NC(C)=O)(COCCC(=O)NCCCCCCOC1OC(CO)C(O)C(O)C1NC(C)=O)COCCC(=O)NCCCCCCOC1OC(CO)C(O)C(O)C1NC(C)=O]

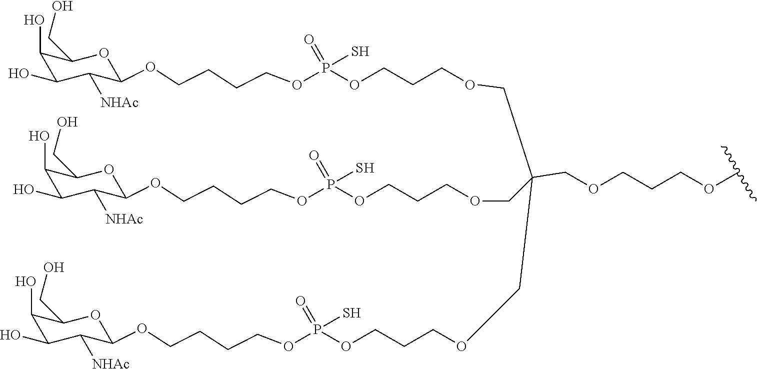 [SMILES: *OCCCOCC(COCCCOP(=O)(S)OCCCCOC1OC(CO)C(O)C(O)C1NC(C)=O)(COCCCOP(=O)(S)OCCCCOC1OC(CO)C(O)C(O)C1NC(C)=O)COCCCOP(=O)(S)OCCCCOC1OC(CO)C(O)C(O)C1NC(C)=O]

or a pharmaceutically acceptable salt thereof,
wherein [symbol] is an attachment point to the sense strand or the antisense strand, or to a conjugate linker conjugated to the sense strand or the antisense strand.

In some embodiments, the ligand has the following structure:

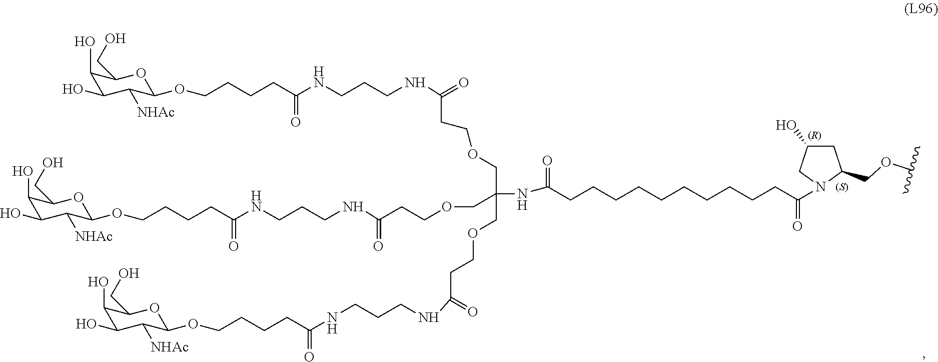

or a pharmaceutically acceptable salt thereof,
wherein [symbol] is an attachment point to the sense strand or the antisense strand, or to a conjugate linker conjugated to the sense strand or the antisense strand.

In some embodiments, the ligand has the following structure:

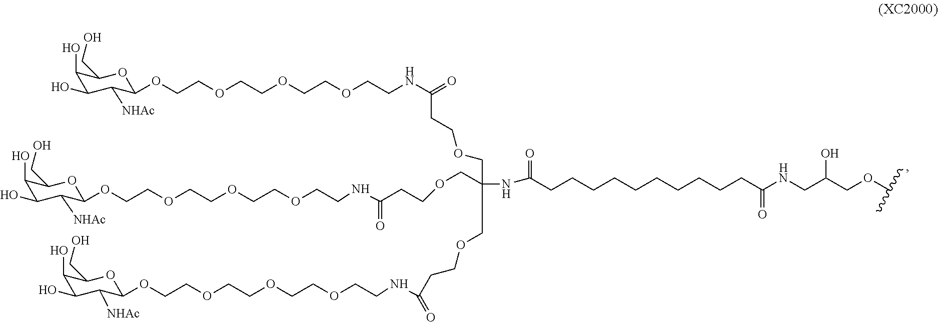

or a pharmaceutically acceptable salt thereof,
wherein [symbol] is an attachment point to the sense strand or the antisense strand, or to a conjugate linker conjugated to the sense strand or the antisense strand.

In certain aspects, the ligand has a structure of Formula (F):

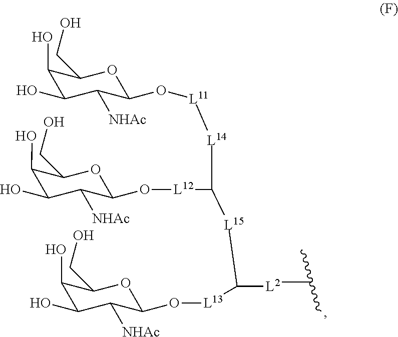

or a pharmaceutically acceptable salt thereof,
wherein:
each L¹¹, L¹², L¹³, L¹⁴, and L¹⁵ is an independently a linker;
L² is a linker as described above;
[symbol] is an attachment point to the sense strand or the antisense strand, or to a conjugate linker conjugated to the sense strand or the antisense strand.

In some embodiments, each L11 is independently a covalent linker having the formula -L11A-L11B-L11C-L11D-L11E-. Each L11A, L11B, L11C, L11D, and L11E is independently a bond, substituted or unsubstituted alkylene, or a substituted or unsubstituted heteroalkylene. In some embodiments, each L11A, L11B, L11C, L11D, and L11E is independently a bond, substituted or unsubstituted alkylene (e.g., C1-C30, C1-C15, or C1-C12 alkylene), or a substituted or unsubstituted heteroalkylene (e.g., 2 to 30, 2 to 15 membered, or 2 to 12 membered heteroalkylene. In some embodiments, each L11A, L11B, L11C, L11D, and L11E is independently a bond, unsubstituted C11-C112 alkylene, —NHC(O)—, —C(O)NH—, —(CH2)a11-O—, —O—(CH2)a11-, —(CH2CH2O)b11-, or —(OCH2CH2)b11-, and each a11 or b11 is independently an integer from 0 to 12.

In some embodiments, each L¹² is independently a covalent linker having the formula -L^{12A}-L^{12B}-L^{12C}-L^{12D}-L^{12E}-. Each L^{12A}, L^{12B}, L^{12C}, L^{12D}, and L^{12E} is independently a bond, substituted or unsubstituted alkylene, or a substituted or unsubstituted heteroalkylene. In some embodiments, each L^{12A}, L^{12B}, L^{12C}, L^{12D}, and L^{12E} is independently a bond, substituted or unsubstituted alkylene (e.g., C₁-C₃₀, C₁-C₁₅, or C₁-C₁₂ alkylene), or a substituted or unsubstituted heteroalkylene (e.g., 2 to 30, 2 to 15 membered, or 2 to 12 membered heteroalkylene. In some embodiments, each L^{12A}, L^{12B}, L^{12C}, L^{12D}, and L^{12E} is independently a bond, unsubstituted C₁₂-C₁₂₂ alkylene, —NHC(O)—, —C(O)NH—, —(CH₂)ₐ₁₂—O—, —O—(CH₂)ₐ₁₂—, —(CH₂CH₂O)_{b12}—, or —(OCH₂CH₂)_{b12}—, and each a12 or b12 is independently an integer from 0 to 12.

In some embodiments, each L13 is independently a covalent linker having the formula -L13A-L13B-L13C-L13D-L13E-. Each L13A, L13B, L13C, L13D, and L13E is independently a bond, substituted or unsubstituted alkylene, or a substituted or unsubstituted heteroalkylene. In some embodiments, each L13A, L13B, L13C, L13D, and L13E is independently a bond, substituted or unsubstituted alkylene (e.g., C1-C30, C1-C15, or C1-C12 alkylene), or a substituted or unsubstituted heteroalkylene (e.g., 2 to 30, 2 to 15 membered, or 2 to 12 membered heteroalkylene. In some embodiments, each L13A, L13B, L13C, L13D, and L13E is independently a bond, unsubstituted C1a14-C12 alkylene, —NHC(O)—, —C(O)NH—, —(CH2)a13-O—, —O—(CH2)a13-, —(CH2CH2O)b13-, or —(OCH2CH2)b13-, and each a13 or b13 is independently an integer from 0 to 12.

In some embodiments, L¹⁴ has the formula -L^{14A}-L^{14B}-L^{14C}-L^{14D}-L^{14E}-. Each L^{14A}, L^{14B} L^{14C}, L^{14D}, and L^{14E} is independently a bond, substituted or unsubstituted alkylene, or a substituted or unsubstituted heteroalkylene. In some embodiments, each L^{14A}, L^{14B}, L^{14C}, L^{14D}, and L^{14E} is independently a bond, unsubstituted C₁-C₁₂ alkylene, —NHC(O)—, —C(O)NH—, —(CH₂)ₐ₁₄—O—, —O—(CH₂)ₐ₁₄—, —(CH₂CH₂O)_{b14}—, or —(OCH₂CH₂)_{b14}—, and each a14 or b14 is independently an integer from 0 to 12. In some embodiments, L¹⁴ is a bond. In some embodiments, L¹⁴ is unsubstituted C₁-C₁₂ alkylene. In some embodiments, L¹⁴ is —C(O)NH—(CH₂)z1, or —NHC(O)—(CH₂)z1 wherein z1 is an integer from 0 to 12.

In some embodiments, L¹⁵ has the formula -L^{15A}-L^{15B}-L^{15C}-L^{15D}-L^{15E}-. Each L^{15A}, L^{15B}, L^{15C}, L^{15D}, and L^{15E} is independently a bond, substituted or unsubstituted alkylene, or a substituted or unsubstituted heteroalkylene. In some embodiments, each L^{15A}, L^{15B}, L^{15C}, L^{15D}, and L^{15E} is independently a bond, unsubstituted C₁-C₁₂ alkylene, —NHC(O)—, —C(O)NH—, —(CH₂)ₐ₁₅—O—, —O—(CH₂)ₐ₁₅—, —(CH₂CH₂O)_{b15}—, or —(OCH₂CH₂)_{b15}—, and each a15 or b15 is independently an integer from 0 to 12. In some embodiments, L¹⁵ is unsubstituted C₁-C₁₂ alkylene. In some embodiments, L¹⁵ is —C(O)NH—(CH₂)z2, or —NHC(O)—(CH₂)z2 wherein z2 is an integer from 0 to 12. In some embodiments, L¹⁵ is —C(O)NH— or —NHC(O)—.

In certain aspects, the ligand includes the following structure:

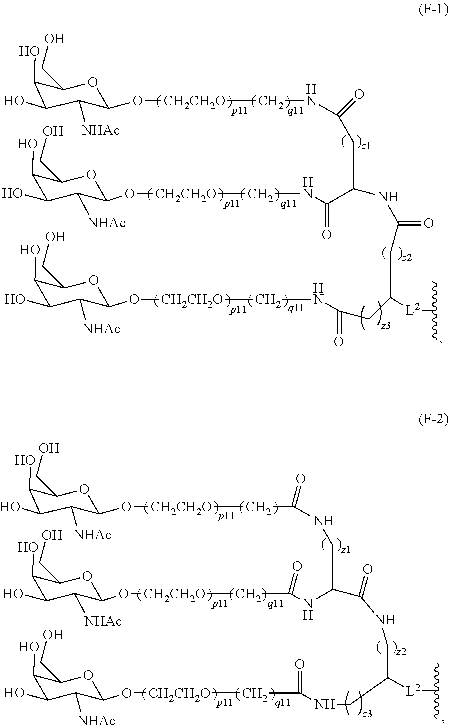

or a pharmaceutically acceptable salt thereof,
wherein:
L² is as described above;
each p11 and q11 is independently an integer from 0 to 12;
each z1, z2, and z3 is independently an integer of 0 to 12; and
[symbol] is an attachment point to the sense strand or the antisense strand, or to a conjugate linker conjugated to the sense strand or the antisense strand.

In some embodiments, the ligand includes the following structures (F-1-a) to (F-1-c):

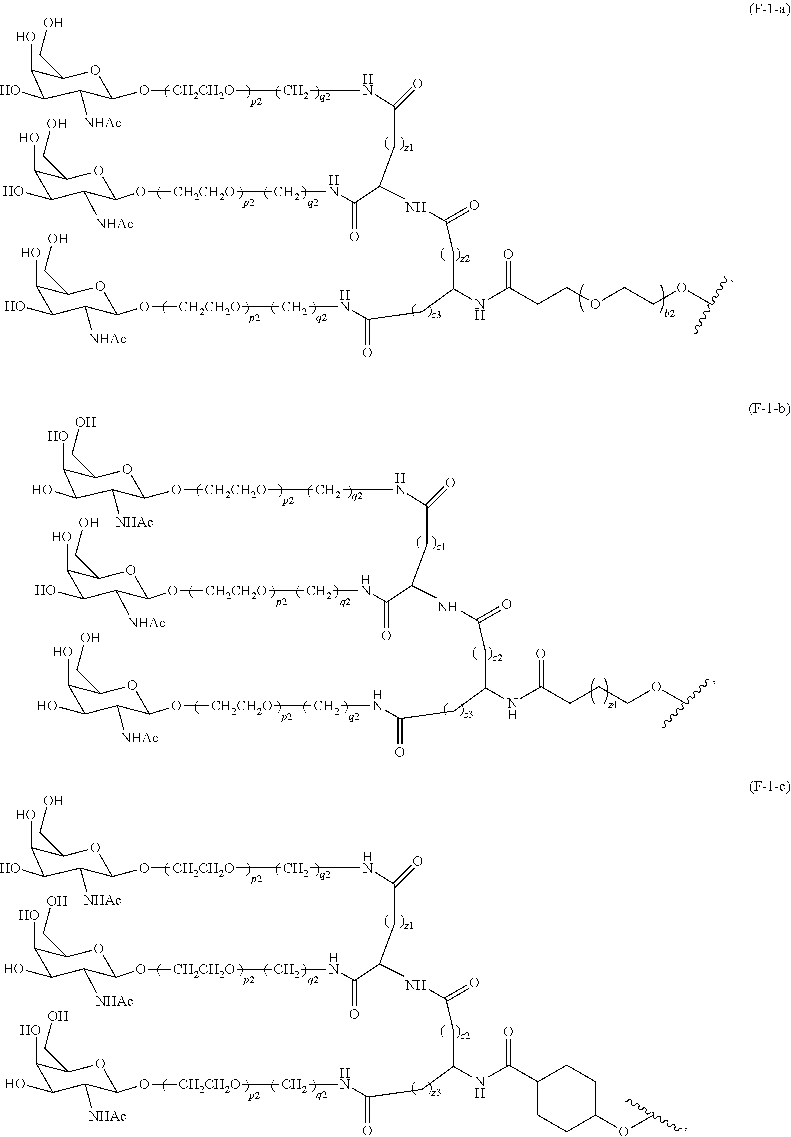

or a pharmaceutically acceptable salt thereof,
wherein W, p2, q2, z1, z2, and z3 are as described above. z4 is an integer from 0 to 10.

In some embodiments, the ligand includes the following structures (F-2-a) to (F-2-c):

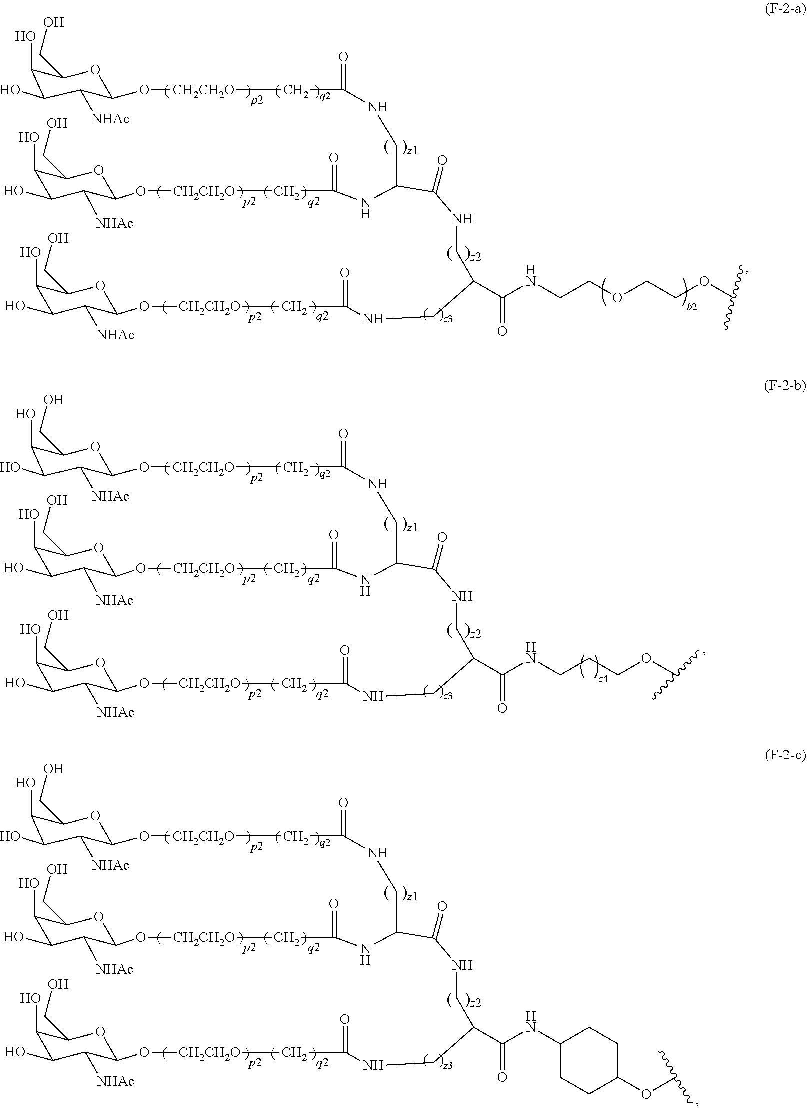

or a pharmaceutically acceptable salt thereof,
wherein W, p2, q2, z1, z2 and z3 are as described above. z4 is an integer from 0 to 10.

In some embodiments, z1 is 0. In some embodiments, z1 is 1. In some embodiments, z1 is 2. In some embodiments, z1 is 3. In some embodiments, z1 is 4. In some embodiments, z2 is 0. In some embodiments, z2 is 1. In some embodiments, z2 is 2. In some embodiments, z2 is 3. In some embodiments, z2 is 4. In some embodiments, z3 is 0. In some embodiments, z3 is 1. In some embodiments, z3 is 2. In some embodiments, z3 is 3. In some embodiments, z3 is 4.

In some embodiments, the ligand includes the following structure:

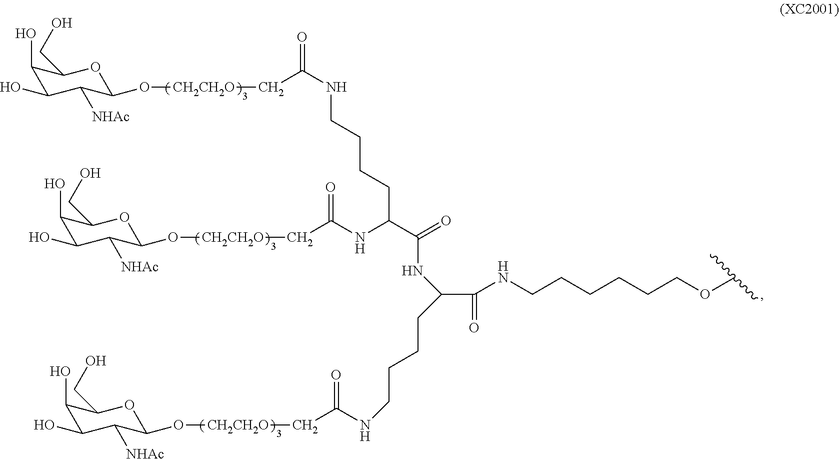

or a pharmaceutically acceptable salt thereof.

Additional suitable ligands related to the above structures of Formula (F) and its subordinates and synthesis thereof are also described in WO2011/104169 and WO2008/022309, entire contents of which are incorporated herein by reference.

In some embodiments, the ligand is coupled or conjugated to the 3′ end of the sense strand. In some embodiments, the ligand is coupled or conjugated to the 5′ end of the sense strand. In some embodiments, the ligand is coupled or conjugated to the 3′ end of the antisense strand. In some embodiments, the ligand is coupled or conjugated to the 5′ end of the antisense strand. In some embodiments, two ligands may be coupled to both sense strand and antisense strand. In some embodiments, the ligand is conjugated to a “non-end” of the sense strand or antisense strand.

In some embodiments, the ligands may be conjugated to the 3′ end of the sense strand and to the 3′ end of the antisense strand. In some embodiments, the ligands may be conjugated to the 5′ end of the sense strand and to the 3′ end of the antisense strand. In some embodiments, the ligands may be conjugated to a non-end of the sense strand and to the 3′ end of the antisense strand. In some embodiments, the ligands may be conjugated to the 3′ end of the sense strand and to a non-end of the antisense strand. In some embodiments, the ligands may be conjugated to the 5′ end of the sense strand and to a non-end of the antisense strand. In some embodiments, the ligands may be conjugated to a non-end of the sense strand and to a non-end of the antisense strand (e.g., nucleobases).

In some embodiments, the dsRNAi agent includes the following structure:

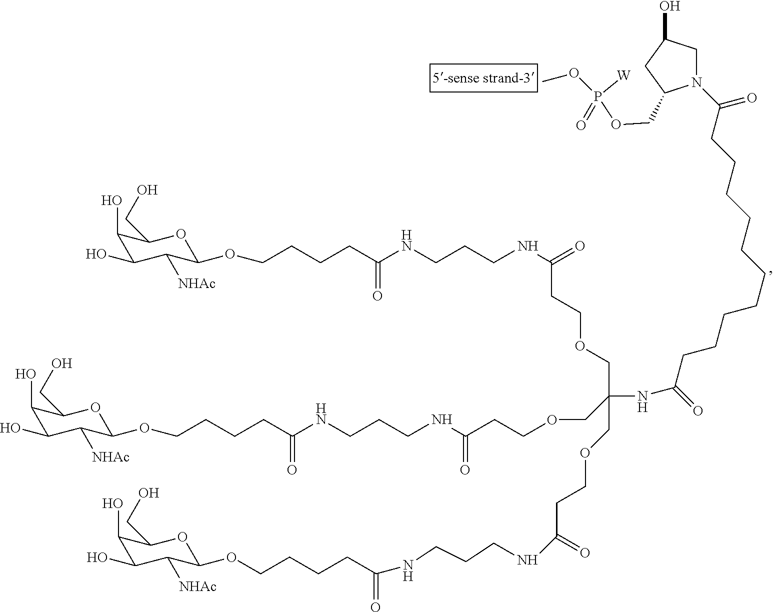

or a pharmaceutically acceptable salt thereof,
wherein W is —OH or —SH.

In some embodiments, the dsRNAi agent includes the following structure:

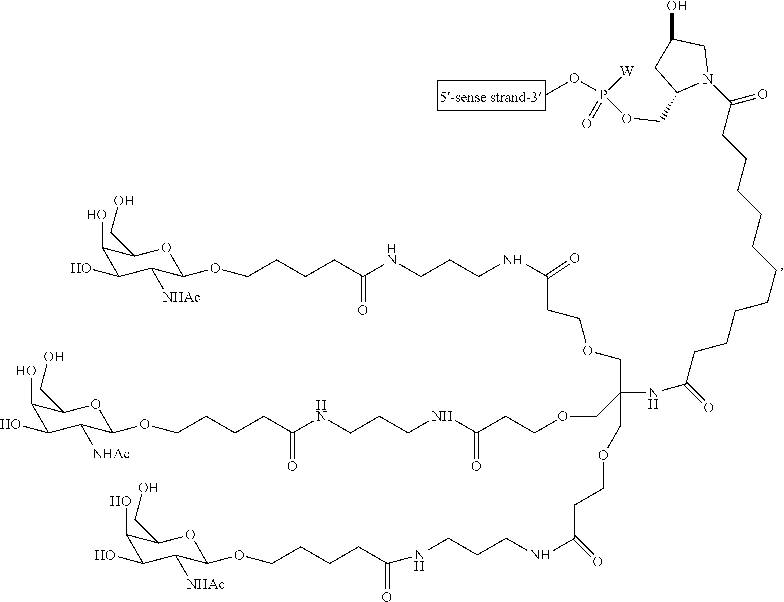

or a pharmaceutically acceptable salt thereof,
wherein W is —OH or —SH, and the sense strand includes any one of the sense strand selected from SEQ ID NOs: 3-381, 761-776, 793-845 and 1013-1018.

In some embodiments, the RNAi agent includes the following structure:

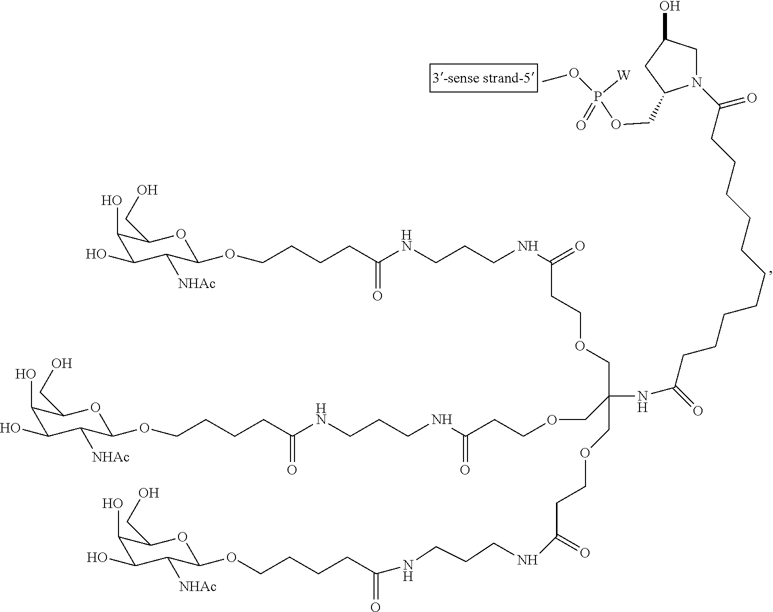

or a pharmaceutically acceptable salt thereof,
wherein W is —OH or —SH.

In some embodiments, the RNAi agent includes the following structure:

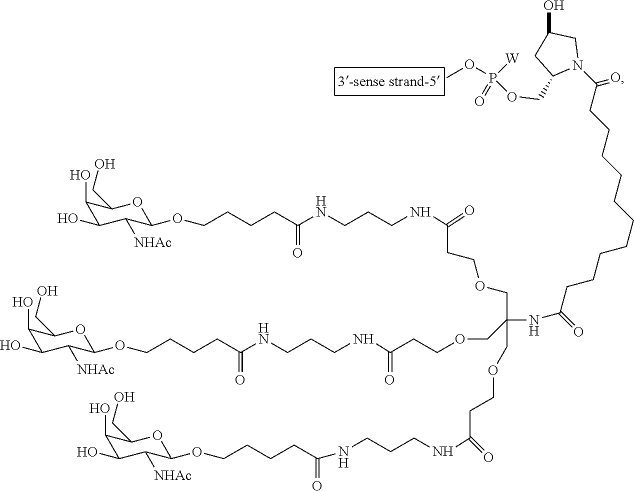

or a pharmaceutically acceptable salt thereof,
wherein W is —OH or —SH, and the sense strand includes any one of the sense strand selected from SEQ ID NOs: 3-381, 761-776, 793-845 and 1013-1018.

In some embodiments, the RNAi agent includes the following structure:

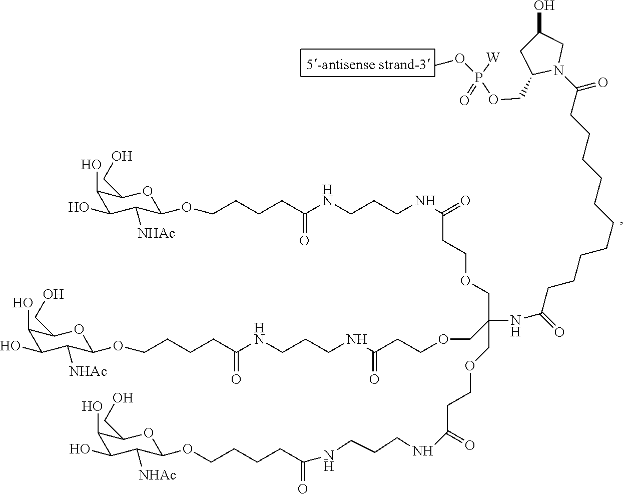

or a pharmaceutically acceptable salt thereof,
wherein W is —OH or —SH.

In some embodiments, the RNAi agent includes the following structure:

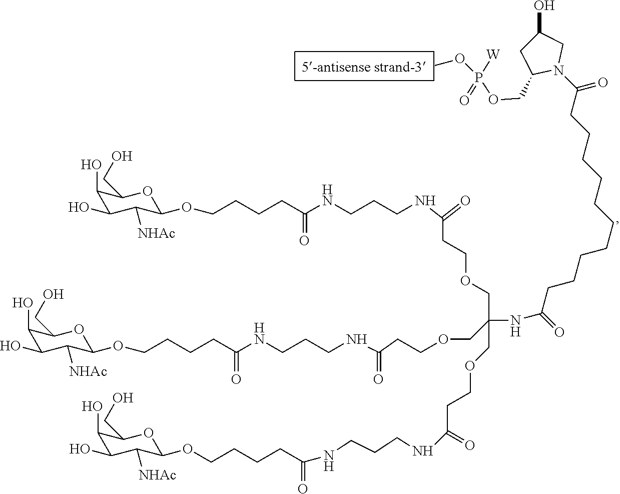

or a pharmaceutically acceptable salt thereof,
wherein W is —OH or —SH, and the antisense strand includes any one of the antisense strand selected from SEQ ID NOs: 382-760, 777-792, 846-898 and 1019-1024.

In some embodiments, the RNAi agent includes the following structure:

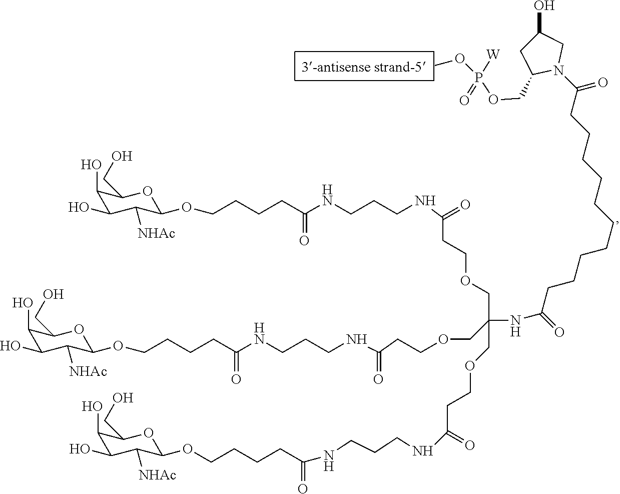

or a pharmaceutically acceptable salt thereof,
wherein W is —OH or —SH.

In some embodiments, the RNAi agent includes the following structure:

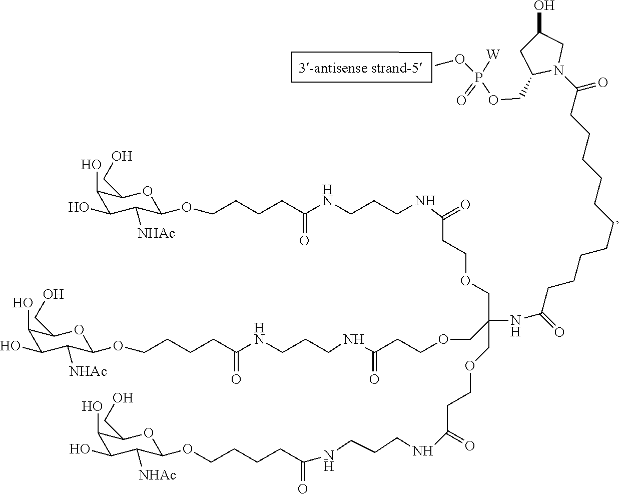

or a pharmaceutically acceptable salt thereof,
wherein W is —OH or —SH and the antisense strand includes any one of the antisense strand selected from SEQ ID NOs: 382-760, 777-792, 846-898 and 1019-1024.

In some embodiments, the RNAi agent includes the following structure:

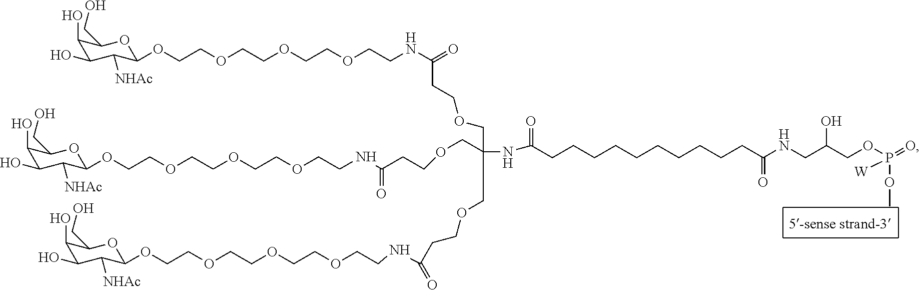

or a pharmaceutically acceptable salt thereof,
wherein W is —OH or —SH.

In some embodiments, the RNAi agent includes the following structure:

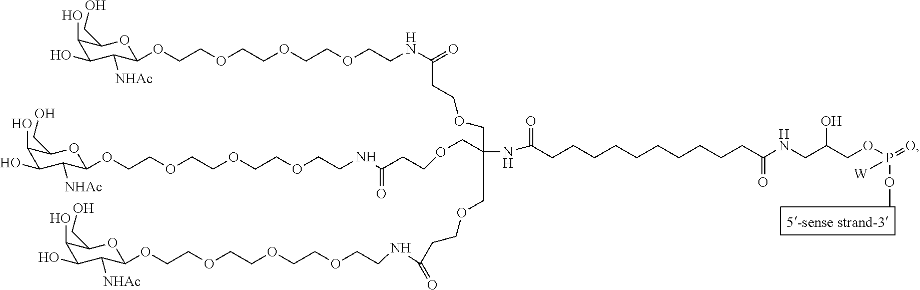

or a pharmaceutically acceptable salt thereof,
wherein W is —OH or —SH and the sense strand includes any one of the sense strand selected from SEQ ID NOs: 3-381, 761-776, 793-845 and 1013-1018.

In some embodiments, the RNAi agent includes the following structure:

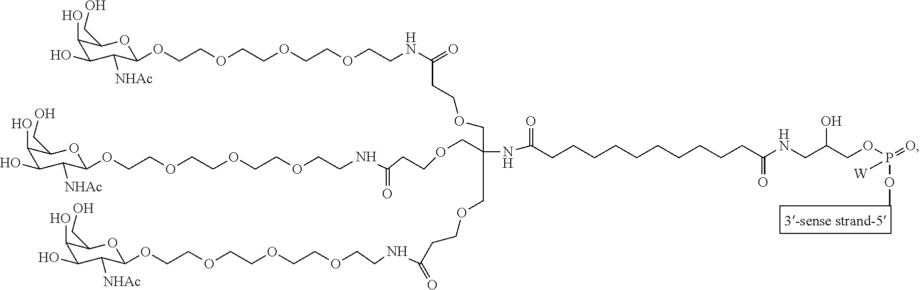

or a pharmaceutically acceptable salt thereof,
wherein W is —OH or —SH.

In some embodiments, the RNAi agent includes the following structure:

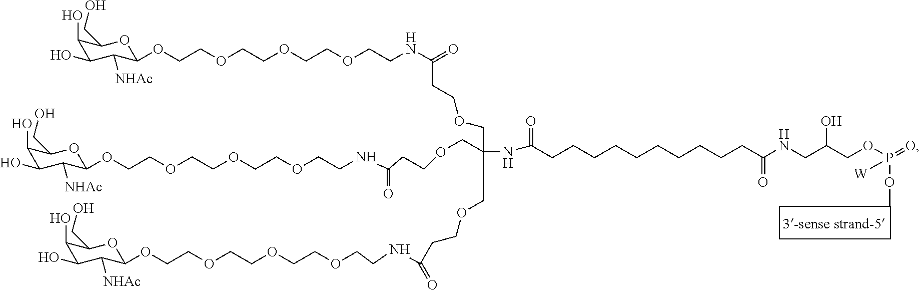

or a pharmaceutically acceptable salt thereof,
wherein W is —OH or —SH and the sense strand includes any one of the sense strand selected from SEQ ID NOs: 3-381, 761-776, 793-845 and 1013-1018.

In some embodiments, the RNAi agent includes the following structure:

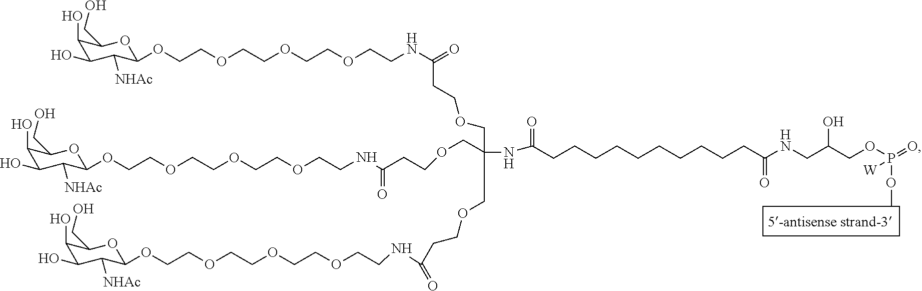

or a pharmaceutically acceptable salt thereof, wherein W is —OH or —SH.

In some embodiments, the RNAi agent includes the following structure:

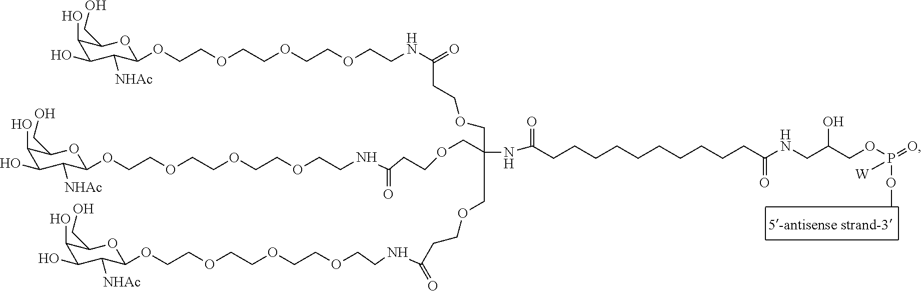

or a pharmaceutically acceptable salt thereof,
wherein W is —OH or —SH and the antisense strand includes any one of the antisense strand selected from SEQ ID NOs: 382-760, 777-792, 846-898 and 1019-1024.

In some embodiments, the RNAi agent includes the following structure:

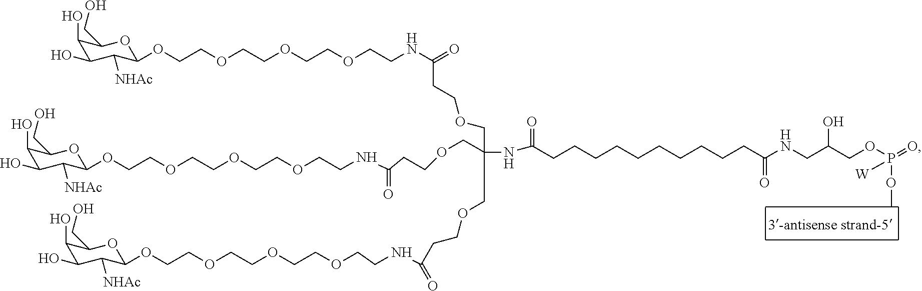

or a pharmaceutically acceptable salt thereof,
wherein W is —OH or —SH.

In some embodiments, the RNAi agent includes the following structure:

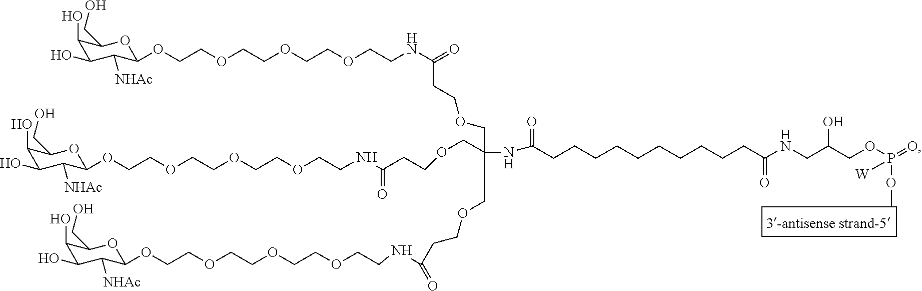

or a pharmaceutically acceptable salt thereof,
wherein W is —OH or —SH and the antisense strand includes any antisense strand selected from SEQ ID NOs: 382-760, 777-792, 846-898 and 1019-1024.

In some embodiments, the RNAi agent includes the following structure:

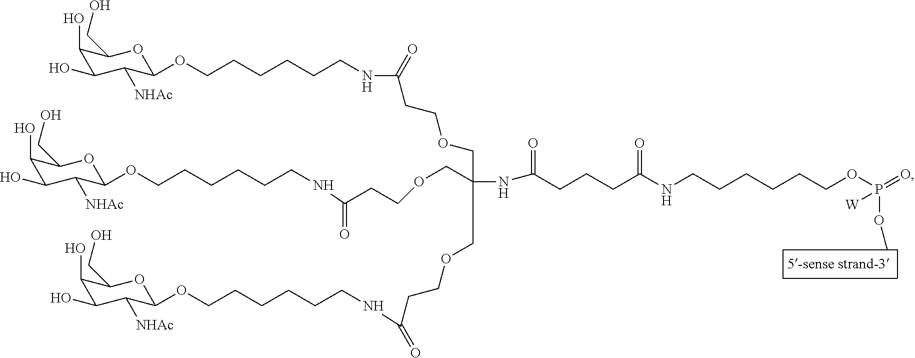

or a pharmaceutically acceptable salt thereof,
wherein W is —OH or —SH.

In some embodiments, the RNAi agent includes the following structure:

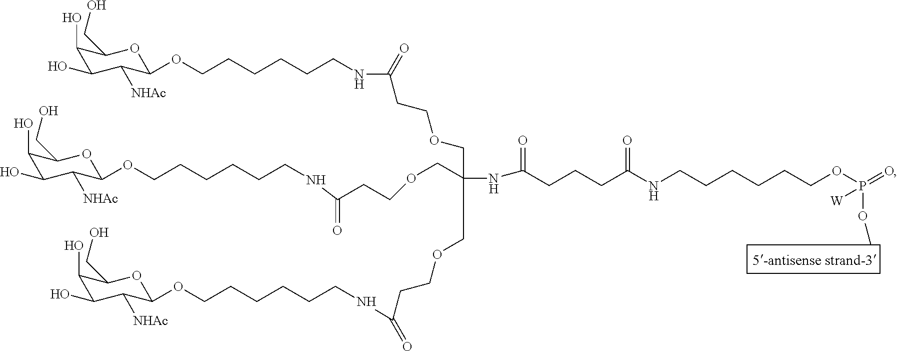

or a pharmaceutically acceptable salt thereof,
wherein W is —OH or —SH and the sense strand includes any one of the sense strand selected from SEQ ID NOs: 3-381, 761-776, 793-845 and 1013-1018.

In some embodiments, the RNAi agent includes the following structure:

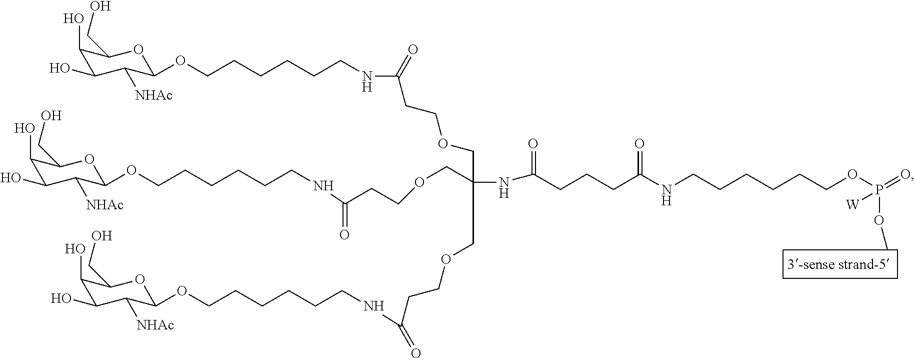

or a pharmaceutically acceptable salt thereof,
wherein W is —OH or —SH.

In some embodiments, the RNAi agent includes the following structure:

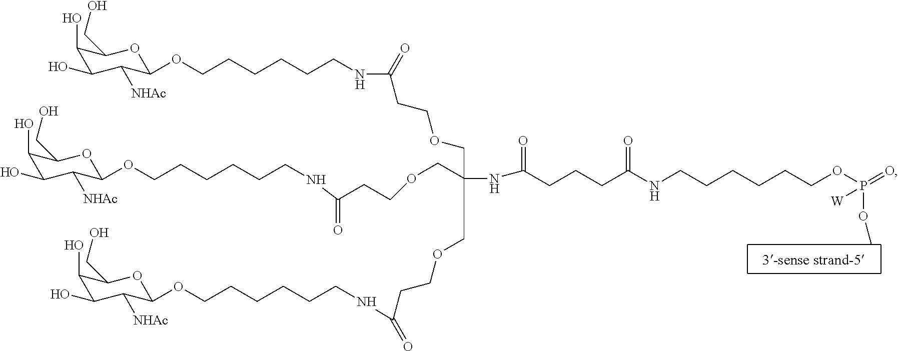

or a pharmaceutically acceptable salt thereof,
wherein W is —OH or —SH and the sense strand includes any one of the sense strand selected from SEQ ID NOs: 3-381, 761-776, 793-845 and 1013-1018.

In some embodiments, the RNAi agent includes the following structure:

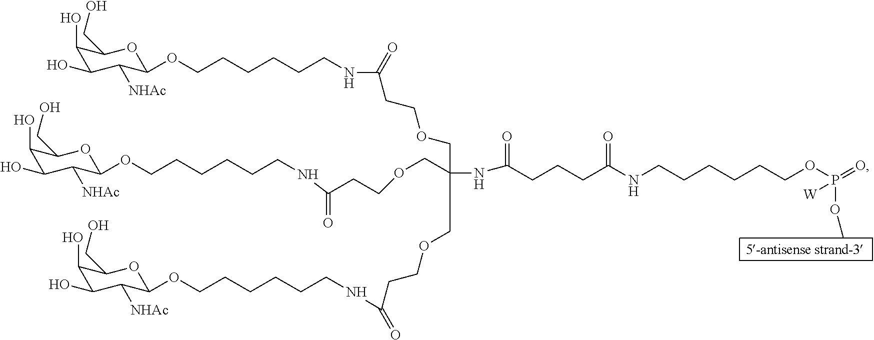

or a pharmaceutically acceptable salt thereof,
wherein W is —OH or —SH.

In some embodiments, the RNAi agent includes the following structure:

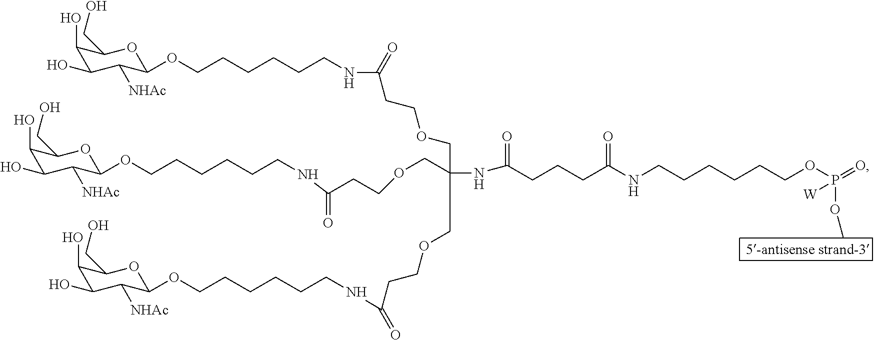

or a pharmaceutically acceptable salt thereof,
wherein W is —OH or —SH and the antisense strand includes any one of the antisense strand selected from SEQ ID NOs: 382-760, 777-792, 846-898 and 1019-1024.

In some embodiments, the RNAi agent includes the following structure:

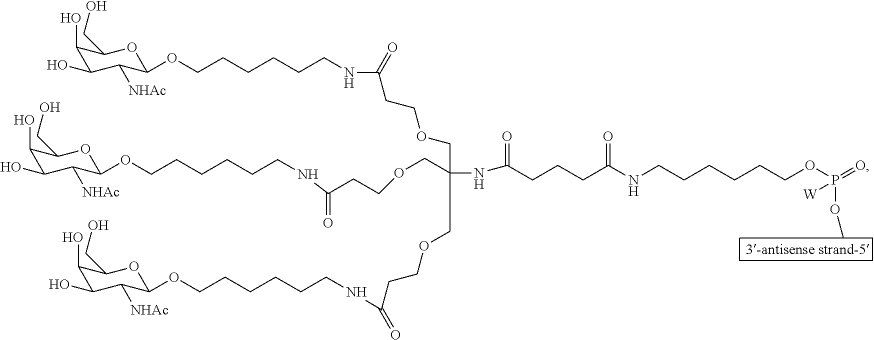

or a pharmaceutically acceptable salt thereof,
wherein W is —OH or —SH.

In some embodiments, the RNAi agent includes the following structure:

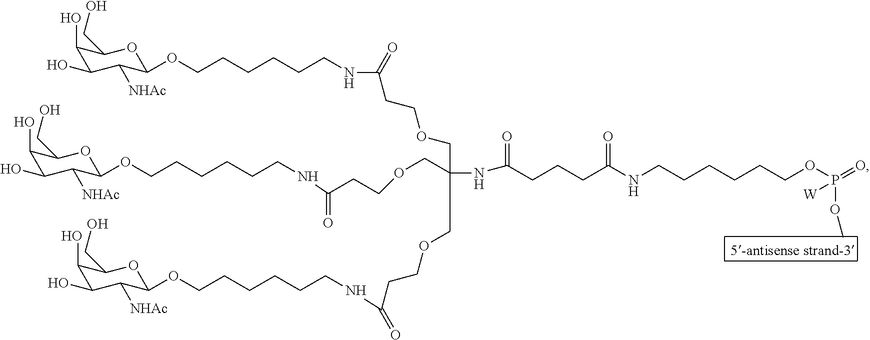

or a pharmaceutically acceptable salt thereof,
wherein W is —OH or —SH and the antisense strand includes any one of the antisense strand selected from SEQ ID NOs: 382-760, 777-792, 846-898 and 1019-1024.

In some embodiments, the RNAi agent includes the following structure:

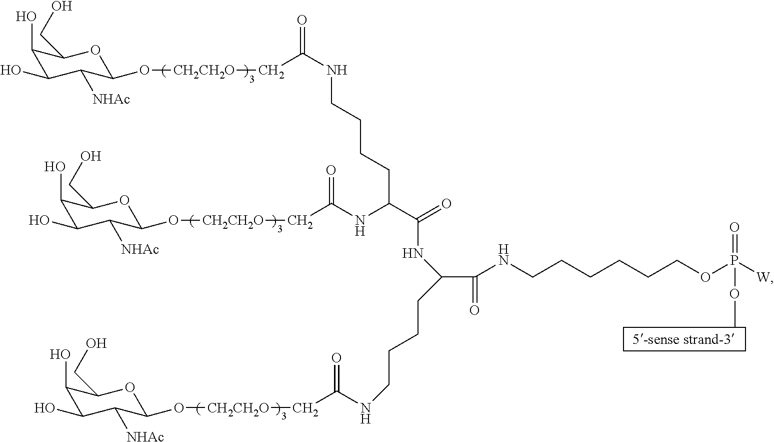

or a pharmaceutically acceptable salt thereof,
wherein W is —OH or —SH.

In some embodiments, the RNAi agent includes the following structure:

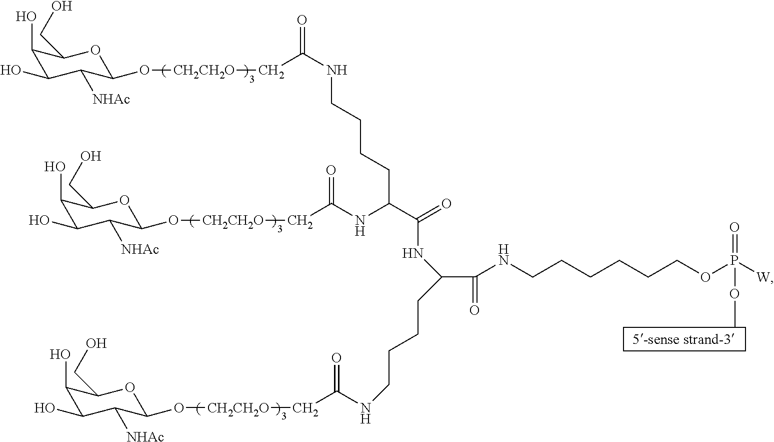

or a pharmaceutically acceptable salt thereof,
wherein W is —OH or —SH and the sense strand includes any one of the sense strand selected from SEQ ID NOs: 3-381, 761-776, 793-845 and 1013-1018.

In some embodiments, the RNAi agent includes the following structure:

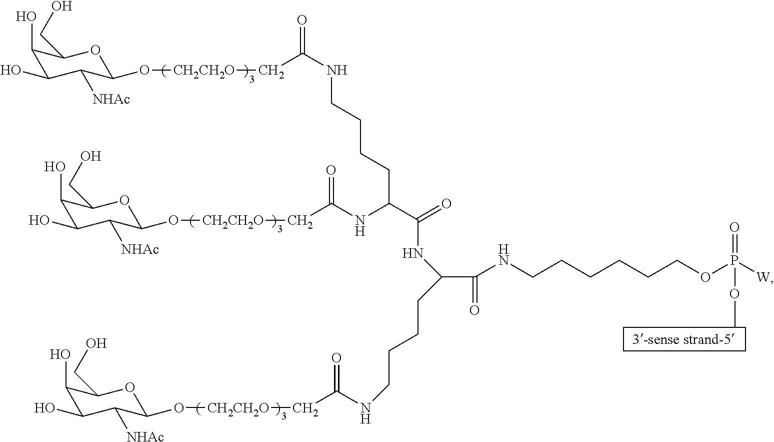

or a pharmaceutically acceptable salt thereof,
wherein W is —OH or —SH.

In some embodiments, the RNAi agent includes the following structure:

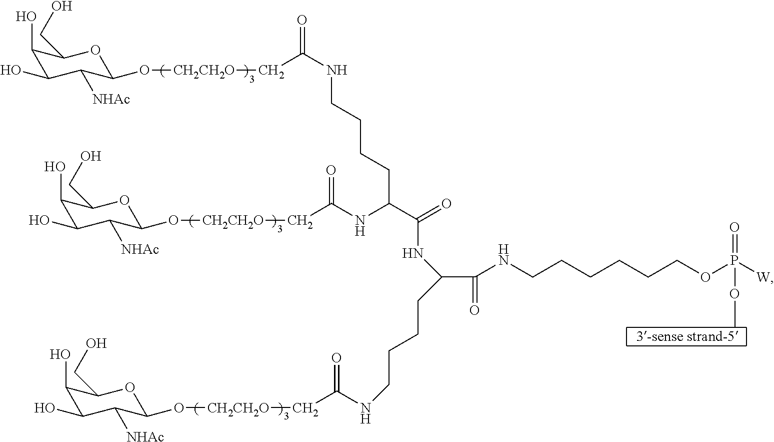

or a pharmaceutically acceptable salt thereof,
wherein W is —OH or —SH and the sense strand includes any one of the sense strand selected from SEQ ID NOs: 3-381, 761-776, 793-845 and 1013-1018.

In some embodiments, the RNAi agent includes the following structure:

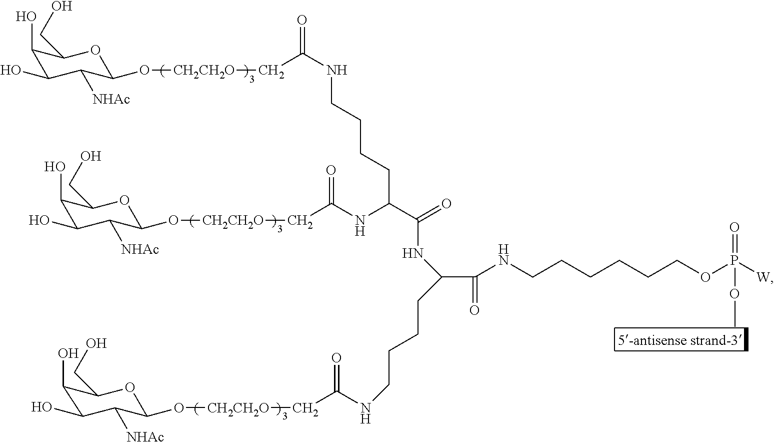

or a pharmaceutically acceptable salt thereof,
wherein W is —OH or —SH.

In some embodiments, the RNAi agent includes the following structure:

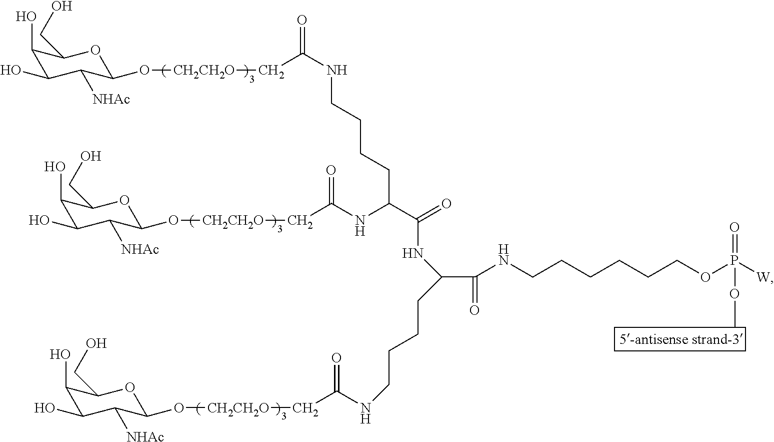

or a pharmaceutically acceptable salt thereof,
wherein W is —OH or —SH and the antisense strand includes any one of the antisense strand selected from SEQ ID NOs: 382-760, 777-792, 846-898 and 1019-1024.

In some embodiments, the RNAi agent includes the following structure:

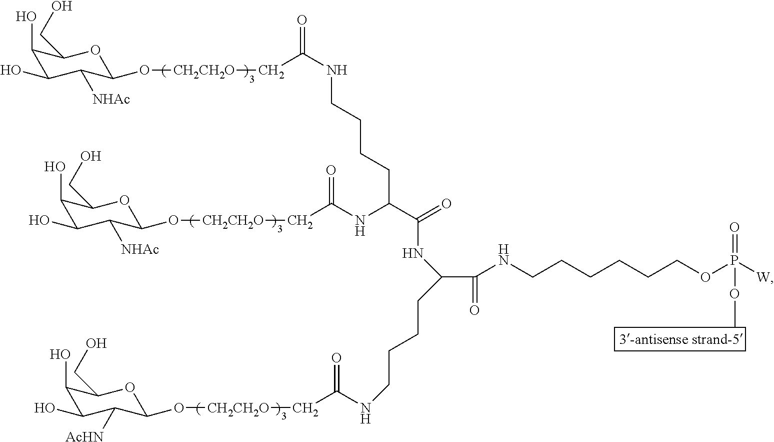

or a pharmaceutically acceptable salt thereof,
wherein W is —OH or —SH.

In some embodiments, the RNAi agent includes the following structure:

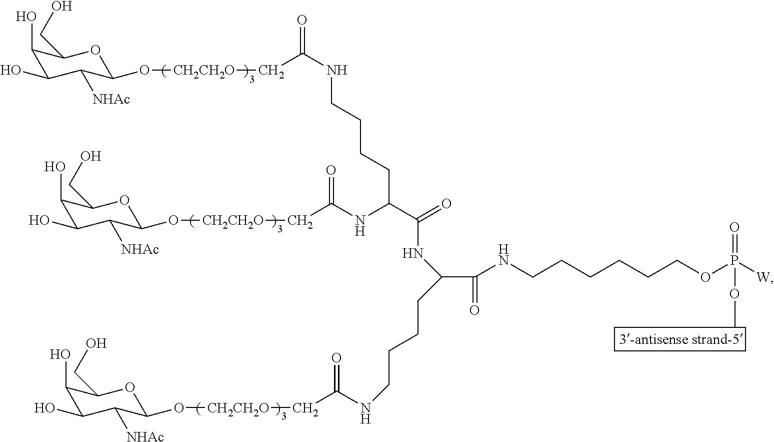

or a pharmaceutically acceptable salt thereof,
wherein W is —OH or —SH, and the antisense strand includes any one of the antisense strand selected from SEQ ID NOs: 382-760, 777-792, 846-898 and 1019-1024.

In some embodiments, W is —OH. In some embodiments, W is —SH.

In certain aspects, the ligand may further include an inverted abasic deoxyribonucleotide (invAb) that may be connected to the sense strand or antisense strand. In some embodiments, the ligand comprises, for example, the following structure:

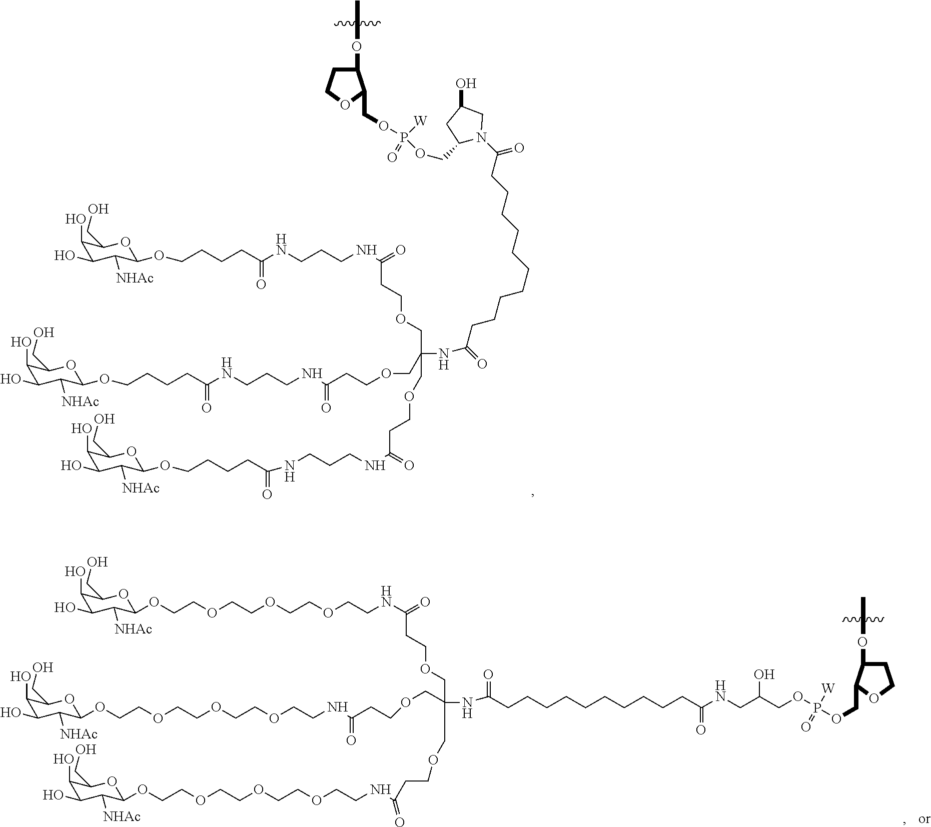

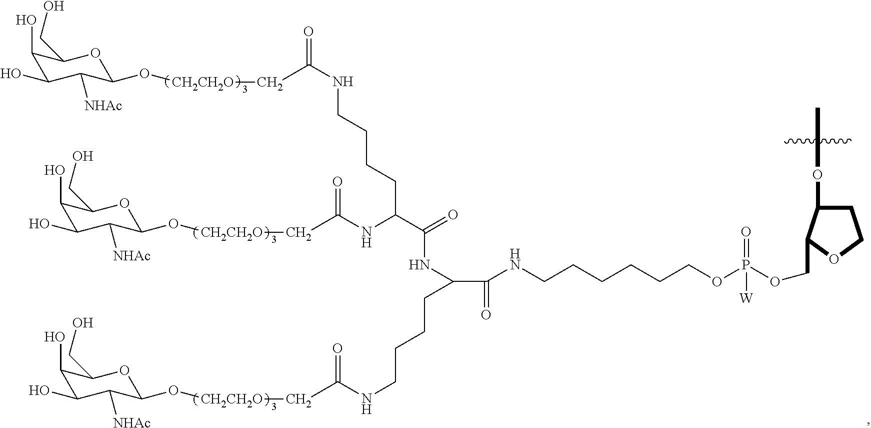

or a pharmaceutically acceptable salt thereof,
wherein W is —OH or —SH, and [symbol] is an attachment point to the sense strand or the antisense strand, or to a conjugate linker conjugated to the sense strand or the antisense strand.

In some embodiments, W is —OH. In some embodiments, W is —SH.

In certain aspects, the ligand as described above may direct the dsRNAi to a specific cell or tissue, e.g., liver cells. In some embodiments, the ligand may direct the dsRNAi to a liver cell.

Examples of PCKS9 targeting dsRNAi agent including the liver targeting ligand (e.g., XC2000 or L96) are listed in Table 6.

TABLE 6

| | Site | | | | |
|---|---|---|---|---|---|
| | of | | SEQ | | SEQ |
| SiRNA | mRNA | | ID | | ID |
| No. | target | Sense strand - modified | NO. | Antisense strand - modified | NO. |
| D1 | 3551 | C004p001U004p001A004pG0 | 899 | A004p001C007p001A004pA00 | 956 |
| | | 04pA004pC004pC007pU004p | | 7pA007pA007pG004pC007pA0 | |
| | | G007pU004pT002pU004pU00 | | 04pA007pA004pA007pC004pA | |
| | | 4pG004pC004pU004pU004pU | | 007pG004pG007pU004pC007p | |
| | | 004pU004pG004pU004pX200 | | U004pA004pG004p001A004p0 | |
| | | 0 | | 01A004 | |
| D2 | 3551 | C004p001U004p001A004pG0 | 900 | | |
| | | 04pA004pC004pC007pU004p | | 4pA004pA007pG004pC004pA0 | |
| | | G007pU007pT002pU004pU00 | | 04pA004pA004pA004pC004pA | |
| | | 4pG004pC004pU004pU004pU | | 007pG004pG007pU004pC004p | |
| | | 004pU004pG004pU004pX200 | | U004pA004pG004p001A004p0 | |
| | | 0 | | 01A004 | |
| D3 | 3551 | C004p001U004p001A004pG0 | 901 | X033A1027p001C007p001A00 | 958 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG004pU004pX200 | | C004pU004pA004pG004p001A | |
| | | 0 | | 004p001A004 | |
| D4 | 3551 | C005p001T005p001A004pGO | 902 | X033A1027p001C007p001A00 | 959 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG005pT005pX200 | | C004pU004pA004pG004p001A | |
| | | 0 | | 004p001A004 | |
| D5 | 3551 | C005p001T005p001A004pG0 | 903 | A004p001C007p001A004pA00 | 960 |
| | | 04pA004pC004pC007pU004p | | 4pA004pA007pG004pC004pA0 | |
| | | G007pU007pT002pU004pU00 | | 04pA004pA004pA004pC004pA | |
| | | 4pG004pC004pU004pU004pU | | 007pG004pG007pU004pC004p | |
| | | 004pU004pG005pT005pX200 | | U004pA004pG004p001A004p0 | |
| | | 0 | | 01A004 | |
| D6 | 3551 | C005p001T005p001A004pG0 | 904 | X033U1027p001C007p001A00 | 961 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG005pA005pX200 | | C004pU004pA004pG004p001A | |
| | | 0 | | 005p001A005 | |
| D7 | 3551 | C004p001U004p001A004pG0 | 905 | A004p001C007p001A004pA00 | 962 |
| (D1- | | 04pA004pC004pC007pU004p | | 7pA007pA007pG004pC007pA0 | |
| 1) | | G007pU004pT002pU004pU00 | | 04pA007pA004pA007pC004pA | |
| | | 4pG004pC004pU004pU004pU | | 007pG004pG007pU004pC007p | |
| | | 004pU004pG004pU004pX108 | | U004pA004pG004p001A004p0 | |
| | | 5 | | 01A004 | |
| D8 | 3551 | C005p001T005p001A004pG0 | 906 | X033A1027p001C007p001A00 | 963 |
| (D4- | | 04pA004pC004pC007pU004p | | 4pA004pA004pA007pG004pC0 | |
| 1) | | G007pU007pT002pU004pU00 | | 04pA004pA004pA004pA004pC | |
| | | 4pG004pC004pU004pU004pU | | 004pA007pG004pG007pU004p | |
| | | 004pU004pG005pT005pX108 | | C004pU004pA004pG004p001A | |
| | | 5 | | 004p001A004 | |
| D9 | 3551 | C005p001T005p001A004pG0 | 907 | A004p001C007p001A004pA00 | 964 |
| (D5- | | 04pA004pC004pC007pU004p | | 4pA004pA007pG004pC004pA0 | |
| 1) | | G007pU007pT002pU004pU00 | | 04pA004pA004pA004pC004pA | |
| | | 4pG004pC004pU004pU004pU | | 007pG004pG007pU004pC004p | |
| | | 004pU004pG005pT005pX108 | | U004pA004pG004p001A004p0 | |
| | | 5 | | 01A004 | |
| D10 | 3551 | C004p001U004p001A004pG0 | 908 | X033A1027p001C007p001A00 | 965 |
| (D3- | | 04pA004pC004pC007pU004p | | 4pA004pA004pA007pG004pC0 | |
| 1) | | G007pU007pT002pU004pU00 | | 04pA004pA004pA004pA004pC | |
| | | 4pG004pC004pU004pU004pU | | 004pA007pG004pG007pU004p | |
| | | 004pU004pG004pU004pX108 | | C004pU004pA004pG004p001A | |
| | | 5 | | 004p001A004 | |
| D11 | 3551 | C005p001T005p001A004pG0 | 909 | X033U1027p001C007p001A00 | 966 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG005pA005pX200 | | C004pU004pA004pG004p001A | |
| | | 0 | | 004p001A004 | |
| D12 | 3551 | C005p001T005p001A004pG0 | 910 | X033U1027p001C007p001A00 | 967 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001A00 | | C004pU004pA004pG004p001A | |
| | | 5pX2000 | | 004p001A004 | |
| D13 | 3551 | C005p001T005p001A004pG0 | 911 | X033A1027p001C007p001A00 | 968 |
| | | 04pA004pC004pC007pU004p | | 4pA004pA004pA007pG004pC0 | |
| | | G007pU007pU007pU004pU0 | | 04pA004pA004pA004pA004pC | |
| | | 04pG004pC004pU004pU004p | | 004pA007pG004pG007pU004p | |
| | | U004pU004pG005pT005pX20 | | C004pU004pA004pG004p001A | |
| | | 00 | | 004p001A004 | |
| D14 | 3551 | C005p001T005p001A004pGO | 912 | X033A1027p001C007p001A00 | 969 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | pX2000 | | 004p001A004 | |
| D15 | 3551 | C005p001T005p001A004pG0 | 913 | X033A1027p001C007p001A00 | 970 |
| | | 04pA004pC004pC007pU004p | | 4pA004pA004pA007pG004pC0 | |
| | | G007pU007pU007pU004pU0 | | 04pA004pA004pA004pA004pC | |
| | | 04pG004pC004pU004pU004p | | 004pA007pG004pG007pU004p | |
| | | U004pU004p001G005p001TO | | C004pU004pA004pG004p001A | |
| | | 05pX2000 | | 004p001A004 | |
| D16 | 3551 | C005p001T005p001A004pG0 | 914 | A004p001C007p001A004pA00 | 971 |
| | | 04pA004pC004pC007pU004p | | 4pA004pA007pG004pC004pA0 | |
| | | G007pU007pT002pU004pU00 | | 04pA004pA004pA004pC004pA | |
| | | 4pG004pC004pU004pU004pU | | 007pG004pG007pU004pC004p | |
| | | 004pU004p001G005p001T005 | | U004pA004pG004p001A004p0 | |
| | | pX2000 | | 01A004 | |
| D17 | 3551 | C005p001T005p001A004pG0 | 915 | A004p001C007p001A004pA00 | 972 |
| | | 04pA004pC004pC007pU004p | | 4pA004pA007pG004pC004pA0 | |
| | | G007pU007pU007pU004pU0 | | 04pA004pA004pA004pC004pA | |
| | | 04pG004pC004pU004pU004p | | 007pG004pG007pU004pC004p | |
| | | U004pU004p001G005p001TO | | U004pA004pG004p001A004p0 | |
| | | 05pX2000 | | 01A004 | |
| D18 | 3551 | C005p001T005p001A004pG0 | 916 | X033A1027p001C007p001A04 | 973 |
| | | 04pA004pC004pC007pU004p | | 2pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG005pT005pX200 | | C004pU004pA004pG004p001A | |
| | | 0 | | 004p001A004 | |
| D19 | 3551 | C005p001T005p001A004pG0 | 917 | X033A1027p001C007p001A04 | 974 |
| | | 04pA004pC004pC007pU004p | | 2pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | pX2000 | | 004p001A004 | |
| D20 | 3551 | C042p001U042p001A004pG0 | 918 | X033A1027p001C007p001A00 | 975 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG042pU042pX200 | | C004pU004pA004pG004p001A | |
| | | 0 | | 004p001A004 | |
| D21 | 3551 | C042p001U042p001A004pG0 | 919 | X033A1027p001C007p001A00 | 976 |
| | | 04pA004pC004pC007pU004p | | 4pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G042p001U04 | | C004pU004pA004pG004p001A | |
| | | 2pX2000 | | 004p001A004 | |
| D22 | 3551 | C042p001U042p001A004pG0 | 920 | X033A1027p001C007p001A04 | 977 |
| | | 04pA004pC004pC007pU004p | | 2pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004pG042pU042pX200 | | C004pU004pA004pG004p001A | |
| | | 0 | | 004p001A004 | |
| D23 | 3551 | C042p001U042p001A004pG0 | 921 | X033A1027p001C007p001A04 | 978 |
| | | 04pA004pC004pC007pU004p | | 2pA004pA004pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G042p001U04 | | C004pU004pA004pG004p001A | |
| | | 2pX2000 | | 004p001A004 | |
| D24 | 3492 | C005p001A005p001A004pG0 | 922 | X033U1027p001A007p001A00 | 979 |
| | | 04pC004pA004pG007pA004p | | 4pA004pA004pG007pA004pU0 | |
| | | C007pA007pU007pU004pU00 | | 04pA004pA004pA004pU004pG | |
| | | 4pA004pU004pC004pU004pU | | 004pU007pC004pU007pG004p | |
| | | 004pU004pT005pA005pX200 | | C004pU004pU004pG004p001C | |
| | | 0 | | 004p001U004 | |
| D25 | 3543 | C005p001A005p001A004pC0 | 923 | X033A1027p001A007p001A00 | 980 |
| | | 04pU004pU004pU007pU004p | | 4pA004pC004pA007pG004pG0 | |
| | | C007pU007pA007pG004pA00 | | 04pU004pC004pU004pA004pG | |
| | | 4pC004pC004pU004pG004pU | | 004pA007pA004pA007pA004p | |
| | | 004pU004pT005pT005pX200 | | G004pU004pU004pG004p001G | |
| | | 0 | | 004p001C004 | |
| D26 | 3564 | G005p001C005p001U004pU0 | 924 | X033A1027p001U007p001A00 | 981 |
| | | 04pU004pU004pG007pU004p | | 4pU004pC004pU007pU004pC0 | |
| | | A007pA007pC007pU004pU00 | | 04pA004pA004pG004pU004pU | |
| | | 4pG004pA004pA004pG004p | | 004pA007pC004pA007pA004p | |
| | | A004pU004pA005pT005pX20 | | A004pA004pG004pC004p001A | |
| | | 00 | | 004p001A004 | |
| D27 | 713 | T005p001T005p001G004pA0 | 925 | X033U1027p001U007p001C00 | 982 |
| | | 04pA004pG004pU007pU004p | | 4pG004pA004pC007pA004pU0 | |
| | | G007pC007pC007pC004pC00 | | 04pG004pG004pG004pG004pC | |
| | | 4pA004pU004pG004pU004pC | | 004pA007pA004pC007pU004p | |
| | | 004pG004pA005pA005pX200 | | U004pC004pA004pA004p001G | |
| | | 0 | | 004p001G004 | |
| D28 | 1102 | C005p001C005p001U004pC0 | 926 | X033A1027p001U007p001A00 | 983 |
| | | 04pA004pU004pA007pG004p | | 4pA004pA004pC007pU004pC0 | |
| | | G007pC007pC007pU004pG00 | | 04pC004pA004pG004pG004pC | |
| | | 4pG004pA004pG004pU004p | | 004pC007pU004pA007pU004p | |
| | | U004pU004pA005pT005pX20 | | G004pA004pG004pG004p001G | |
| | | 00 | | 004p001U004 | |
| D29 | 3251 | A005p001A005p001G004pC0 | 927 | X033A1027p001A007p001G00 | 984 |
| | | 04pC004pA004pA007pG004p | | 4pU004pA004pA007pG004pA0 | |
| | | C007pC007pU007pC004pU00 | | 04pA004pG004pA004pG004pG | |
| | | 4pU004pC004pU004pU004pA | | 004pC007pU004pU007pG004p | |
| | | 004pC004pT005pT005pX200 | | G004pC004p001U004p001U00 | |
| | | 0 | | 4pC004pA004 | |
| D30 | 3252 | A005p001G005p001C004pC0 | 928 | X033U1027p001A007p001A00 | 985 |
| | | 04pA004pA004pG007pC004p | | 4pG004pU004pA007pA004pG0 | |
| | | C007pU007pC007pU004pU00 | | 04pA004pA004pG004pA004pG | |
| | | 4pC004pU004pU004pA004pC | | 004pG007pC004pU007pU004p | |
| | | 004pU004pT005pA005pX200 | | G004pG004pC004pU004p001U | |
| | | 0 | | 004p001C004 | |
| D31 | 3547 | T005p001T005p001U004pU0 | 929 | X033A1027p001A007p001G00 | 986 |
| | | 04pC004pU004pA007pG004p | | 4pC004pA004pA007pA004pA0 | |
| | | A007pC007pC007pU004pG00 | | 04pC004pA004pG004pG004pU | |
| | | 4pU004pU004pU004pU004p | | 004pC007pU004pA007pG004p | |
| | | G004pC004PT005pT005pX20 | | A004pA004p001A004p001A00 | |
| | | 00 | | 4pG004pU004 | |
| D32 | 3553 | A005p001G005p001A004pC0 | 930 | X033U1027p001U007p001A00 | 987 |
| | | 04pC004pU004pG007pU004p | | 4pC004pA004pA007pA004pA0 | |
| | | U007pU007pU007pG004pC00 | | 04pG004pC004pA004pA004pA | |
| | | 4pU004pU004pU004pU004p | | 004pA007pC004pA007pG004p | |
| | | G004pU004pA005pA005pX2 | | G004pU004pC004pU004p001A | |
| | | 000 | | 004p001G004 | |
| D33 | 3568 | T005p001T005p001G004pU0 | 931 | X033A1027p001U007p001A00 | 988 |
| | | 04pA004pA004pC007pU004p | | 4pA004pA004pU007pA004pU0 | |
| | | U007pG007pA007pA004pG0 | | 04pC004pU004pU004pC004pA | |
| | | 04pA004pU004pA004pU004p | | 004pA007pG004pU007pU004p | |
| | | U004pU004pA005pT005pX20 | | A004pC004p001A004p001A00 | |
| | | 00 | | 4pA004pA004 | |
| D34 | 3559 | G005p001T005p001U004pU0 | 932 | X033U1027p001U007p001C00 | 989 |
| | | 04pU004pG004pC007pU004p | | 4pA004pA004pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004pA005pA005pX2 | | A004pA004pA004pC004p001A | |
| | | 000 | | 004p001G004 | |
| D35 | 3547 | T005p001T005p001U004pU0 | 933 | X033A1027p001A007p001G00 | 990 |
| | | 04pC004pU004pA004pG004p | | 4pC004pA004pA007pA004pA0 | |
| | | A007pC007pC007pU004pG00 | | 04pC004pA004pG004pG004pU | |
| | | 4pU004pU004pU004pU004p | | 004pC007pU004pA007pG004p | |
| | | G004pC004p001T005p001TO | | A004pA004p001A004p001A00 | |
| | | 05pX2000 | | 4pG004pU004 | |
| D36 | 3553 | A005p001G005p001A004pC0 | 934 | X033U1027p001U007p001A00 | 99 |
| | | 04pC004pU004pG007pU004p | | 4pC004pA004pA007pA004pA0 | |
| | | U007pU007pU007pG004pC00 | | 04pG004pC004pA004pA004pA | |
| | | 4pU004pU004pU004pU004p | | 004pA007pC004pA007pG004p | |
| | | G004pU004p001A005p001A0 | | G004pU004pC004pU004p001A | |
| | | 05pX2000 | | 004p001G004 | |
| D37 | 3568 | T005p001T005p001G004pU0 | 935 | X033A1027p001U007p001A00 | 992 |
| | | 04pA004pA004pC004pU004p | | 4pA004pA004pU007pA004pU0 | |
| | | U007pG007pA007pA004pG0 | | 04pC004pU004pU004pC004pA | |
| | | 04pA004pU004pA004pU004p | | 004pA007pG004pU007pU004p | |
| | | U004pU004p001A005p001TO | | A004pC004p001A004p001A00 | |
| | | 05pX2000 | | 4pA004pA004 | |
| D38 | 3559 | G005p001T005p001U004pU0 | 936 | X033U1027p001U007p001C00 | 993 |
| | | 04pU004pG004pC007pU004p | | 4pA004pA004pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D39 | 3559 | G005p001T005p001U004pU0 | 937 | X033U1027p001U007p001C00 | 994 |
| | | 04pU004pG004pC007pU004p | | 4pA004pA1016pG007pU004pU | |
| | | U007pU007pU007pG004pU0 | | 004pA004pC004pA004pA004p | |
| | | 04pA004pA004pC004pU004p | | A004pA007pG004pC007pA004 | |
| | | U004pG004p001A005p001A0 | | pA004pA004pA004pC004p001 | |
| | | 05pX2000 | | A004p001G004 | |
| D40 | 3559 | G005p001T005p001U004pU0 | 938 | X033U1027p001U007p001C00 | 995 |
| | | 04pU004pG004pC007pU004p | | 4pA004pA004pG1016pU004pU | |
| | | U007pU007pU007pG004pU0 | | 004pA004pC004pA004pA004p | |
| | | 04pA004pA004pC004pU004p | | A004pA007pG004pC007pA004 | |
| | | U004pG004p001A005p001A0 | | pA004pA004pA004pC004p001 | |
| | | 05pX2000 | | A004p001G004 | |
| D41 | 3559 | G005p001T005p001U004pU0 | 939 | X033U1027p001U007p001C00 | 996 |
| | | 04pU004pG004pC007pU004p | | 4pA004pA004pG007pU1016pU | |
| | | U007pU007pU007pG004pU0 | | 004pA004pC004pA004pA004p | |
| | | 04pA004pA004pC004pU004p | | A004pA007pG004pC007pA004 | |
| | | U004pG004p001A005p001A0 | | pA004pA004pA004pC004p001 | |
| | | 05pX2000 | | A004p001G004 | |
| D42 | 3559 | G005p001T005p001U004pU0 | 940 | X033U1027p001U007p001C00 | 997 |
| | | 04pU004pG004pC007pU004p | | 4pA004pA042pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D43 | 3559 | G005p001T005p001U004pU0 | 941 | X033U1027p001U007p001C00 | 998 |
| | | 04pU004pG004pC007pU004p | | 4pA004pA004pG042pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D44 | 3559 | G005p001T005p001U004pU0 | 942 | X033U1027p001U007p001C00 | 999 |
| | | 04pU004pG004pC007pU004p | | 4pA004pA004pG007pU042pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D45 | 3559 | G005p001T005p001U004pU0 | 943 | X033U1027p001U007p001C00 | 1000 |
| | | 04pU004pG004pC007pU004p | | 4pA004pA002pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D46 | 3559 | G005p001T005p001U004pU0 | 944 | X033U1027p001U007p001C00 | 1001 |
| | | 04pU004pG004pC007pU004p | | 4pA004pA004pG002pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D47 | 3559 | G005p001T005p001U004pU0 | 945 | X033U1027p001U007p001C00 | 1002 |
| | | 04pU004pG004pC007pU004p | | 4pA004pA004pG007pT002pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D48 | 3559 | G005p001T005p001U004pU0 | 946 | X033U1027p001U007p001U00 | 1003 |
| | | 04pU004pG004pC007pU004p | | 4pA004pA004pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pA004p001A005p001A0 | | A004pA004pA004pC004p001A | |
| | | 05pX2000 | | 004p001G004 | |
| D49 | 3551 | A1039p001C005p001T005pA | 1025 | X033A1032p001C007p001A00 | 1031 |
| | | 004pG004pA004pC004pC007 | | 4pA004pA004pA007pG004pC0 | |
| | | pU004pG007pU007pT002pU0 | | 04pA004pA004pA004pA004pC | |
| | | 04pU004pG004pC004pU004p | | 004pA007pG004pG007pU004p | |
| | | U004pU004pU004p001G005p | | C004pU004pA004pG004p001A | |
| | | 001T005pX2000 | | 004p001A004 | |
| D50 | 3551 | A1039p001C005p001T005pA | 1026 | X033A1027p001C007p001A00 | 1032 |
| | | 004pG004pA004pC004pC007 | | 4pA004pA004pA007pG004pC0 | |
| | | pU004pG007pU007pT002pU0 | | 04pA004pA004pA004pA004pC | |
| | | 04pU004pG004pC004pU004p | | 004pA007pG004pG007pU004p | |
| | | U004pU004pU004pG005p001 | | C004pU004pA004pG004p001A | |
| | | T005p001A1039pX2000 | | 004p001A004 | |
| D51 | 3551 | C005p001T005p001A004pG0 | 947 | X033A1027p001C007p001A00 | 1004 |
| (D14- | | 04pA004pC004pC007pU004p | | 4pA004pA004pA007pG004pC0 | |
| 1) | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | pX1085 | | 004p001A004 | |
| D52 | 3551 | C005p001T005p001A004pG0 | 948 | X033A1027p001C007p001A04 | 1005 |
| (D19- | | 04pA004pC004pC007pU004p | | 2pA004pA004pA007pG004pC0 | |
| 1) | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | pX1085 | | 004p001A004 | |
| D53 | 3551 | C042p001U042p001A004pG0 | 949 | X033A1027p001C007p001A00 | 1006 |
| (D21- | | 04pA004pC004pC007pU004p | | 4pA004pA004pA007pG004pC0 | |
| 1) | | G007pU007pT002pU004pU00 | | 04pA004pA004pA004pA004pC | |
| | | 4pG004pC004pU004pU004pU | | 004pA007pG004pG007pU004p | |
| | | 004pU004p001G042p001U04 | | C004pU004pA004pG004p001A | |
| | | 2pX1085 | | 004p001A004 | |
| D54 | 3559 | G005p001T005p001U004pU0 | 950 | X033U1027p001U007p001C00 | 1007 |
| (D38- | | 04pU004pG004pC007pU004p | | 4pA004pA004pG007pU004pU0 | |
| 1) | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX1085 | | 004p001G004 | |
| D55 | 3559 | G005p001T005p001U004pU0 | 951 | X033U1027p001U007p001C04 | 1008 |
| | | 04pU004pG004pC007pU004p | | 2pA004pA004pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D56 | 3559 | G005p001T005p001U004pU0 | 952 | X033U1027p001U007p001C04 | 1009 |
| | | 04pU004pG004pC007pU004p | | 2pA004pA042pG007pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D57 | 3559 | G005p001T005p001U004pU0 | 953 | X033U1027p001U007p001C04 | 1010 |
| | | 04pU004pG004pC007pU004p | | 2pA004pA004pG042pU004pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D58 | 3559 | G005p001T005p001U004pU0 | 954 | X033U1027p001U007p001C04 | 1011 |
| | | 04pU004pG004pC007pU004p | | 2pA004pA004pG007pU042pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D59 | 3559 | G005p001T005p001U004pU0 | 955 | X033U1027p001U007p001C00 | 1012 |
| | | 04pU004pG004pC007pU004p | | 4pA004pA002pG007pT002pU0 | |
| | | U007pU007pU007pG004pU0 | | 04pA004pC004pA004pA004pA | |
| | | 04pA004pA004pC004pU004p | | 004pA007pG004pC007pA004p | |
| | | U004pG004p001A005p001A0 | | A004pA004pA004pC004p001A | |
| | | 05pX2000 | | 004p001G004 | |
| D60 | 3551 | C005p001T005p001A004pG0 | 1027 | X033A1027p001C007p001A04 | 1033 |
| | | 04pA004pC004pC007pU004p | | 2pA004pA042pA007pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | pX1085 | | 004p001A004 | |
| D61 | 3551 | C005p001T005p001A004pG0 | 1028 | X033A1027p001C007p001A04 | 1034 |
| | | 04pA004pC004pC007pU004p | | 2pA004pA004pA042pG004pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | pX1085 | | 004p001A004 | |
| D62 | 3551 | C005p001T005p001A004pG0 | 1029 | X033A1027p001C007p001A04 | 1035 |
| | | 04pA004pC004pC007pU004p | | 2pA004pA004pA007pG042pC0 | |
| | | G007pU007pT002pU004pU00 | | 04pA004pA004pA004pA004pC | |
| | | 4pG004pC004pU004pU004pU | | 004pA007pG004pG007pU004p | |
| | | 004pU004p001G005p001T005 | | C004pU004pA004pG004p001A | |
| | | pX1085 | | 004p001A004 | |
| D63 | 3551 | X2171p001C005p001T005pA | 1030 | X033A1032p001C007p001A00 | 1036 |
| (D49- | | 004pG004pA004pC004pC007 | | 4pA004pA004pA007pG004pC0 | |
| 1) | | pU004pG007pU007pT002pU0 | | 04pA004pA004pA004pA004pC | |
| | | 04pU004pG004pC004pU004p | | 004pA007pG004pG007pU004p | |
| | | U004pU004pU004p001G005p | | C004pU004pA004pG004p001A | |
| | | 001T005pX1085 | | 004p001A004 | |

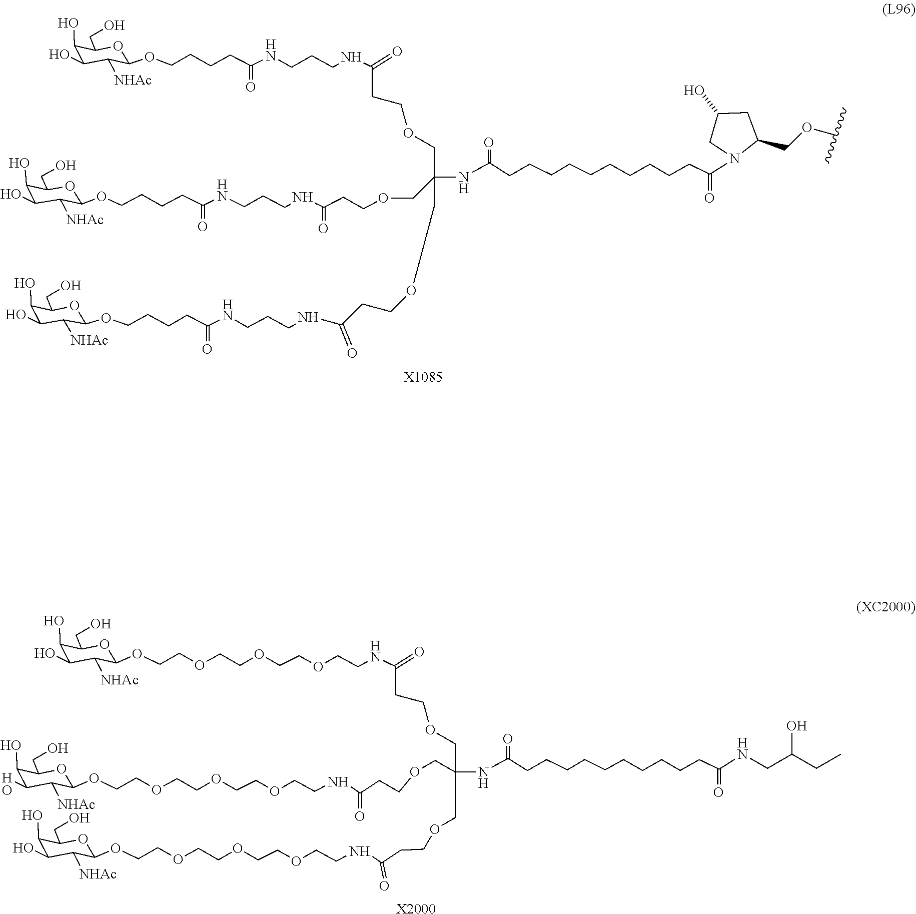

The codes in Table 6 are the same as the codes described in Table A.

Combinations of dsRNA and ligands as described herein are not limited to the examples and embodiments discussed above.

In some embodiments, a dsRNAi agent includes:
(i) a sense strand including SEQ ID NO: 906, and
(ii) an antisense strand including SEQ ID NO: 963;
wherein the ligand (L96) is conjugated to 3′ end of the sense strand to form the following schematic:

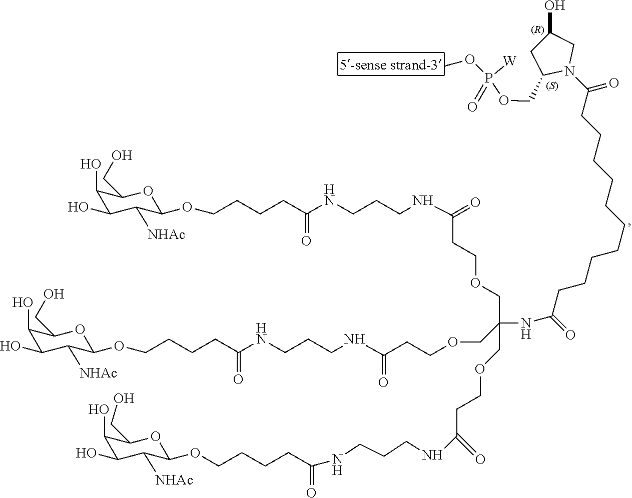

or a pharmaceutically acceptable salt,
wherein W is —OH or —SH.

In some embodiments, a double stranded RNAi agent includes:
(i) a sense strand including SEQ ID NO: 906,
(ii) an antisense strand including SEQ ID NO: 963;
wherein the ligand (L96) is conjugated to 3′ end of the sense strand to form the following schematic:

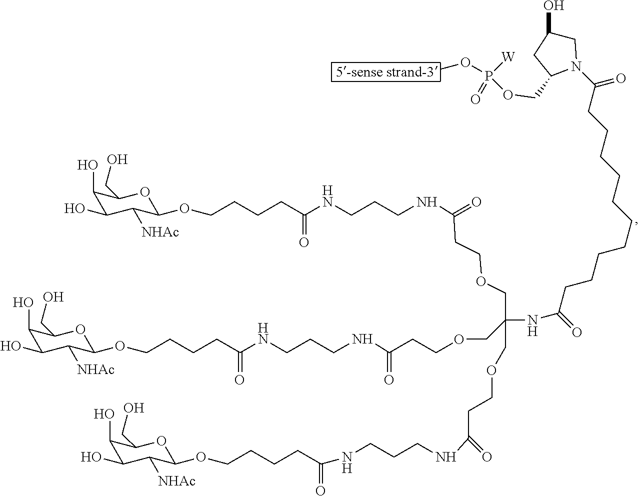

or a pharmaceutically acceptable salt,
wherein W is —OH.

In some embodiments, a dsRNAi agent includes:
(i) a sense strand consisting of SEQ ID NO: 906, and
(ii) an antisense strand consisting of SEQ ID NO: 963;
wherein the ligand (L96) is conjugated to 3′ end of the sense strand to form the following schematic:

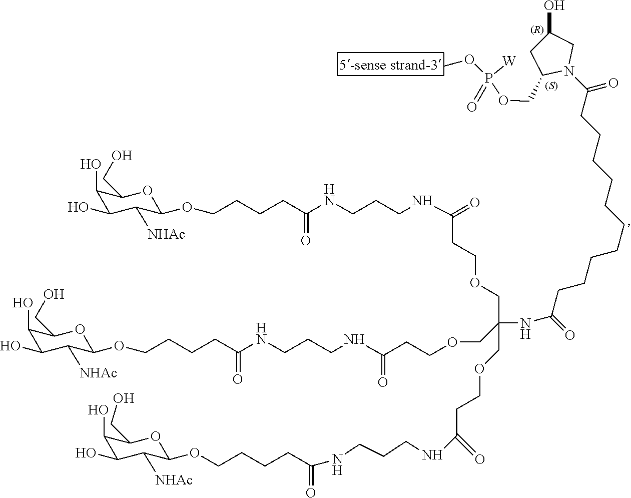

or a pharmaceutically acceptable salt,
wherein W is —OH or —SH.

In some embodiments, a double stranded RNAi agent includes:
(i) a sense strand consisting of SEQ ID NO: 906,
(ii) an antisense strand consisting of SEQ ID NO: 963;
wherein the ligand (L96) is conjugated to 3′ end of the sense strand to form the following schematic:

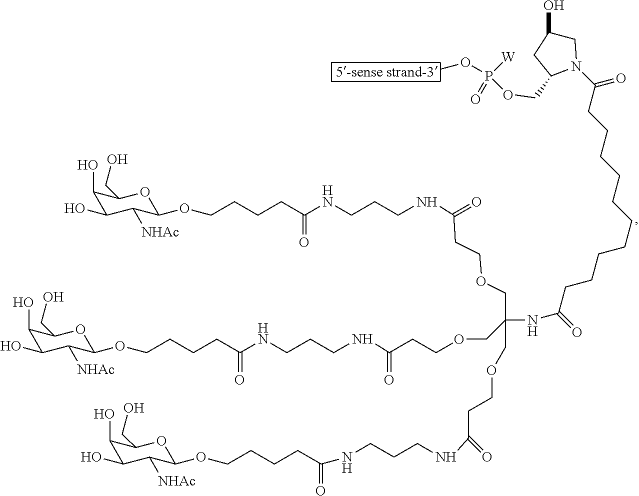

or a pharmaceutically acceptable salt,
wherein W is —OH.

In some embodiments, a dsRNAi agent includes:
(i) a sense strand including SEQ ID NO: 907, and
(ii) an antisense strand including SEQ ID NO: 964;
wherein the ligand (L96) is conjugated to 3′ end of the sense strand to form the following schematic:

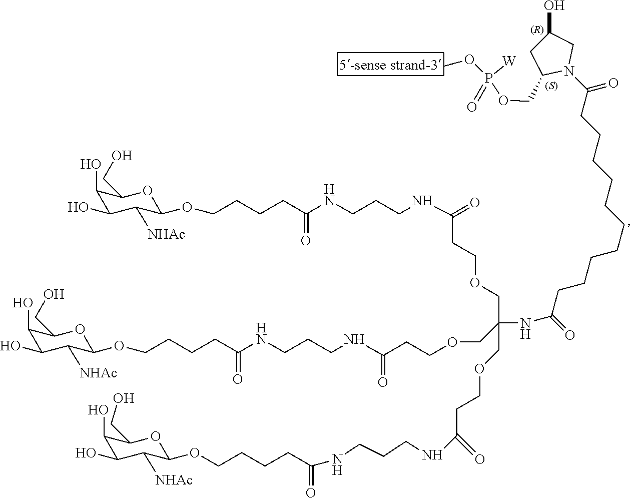

or a pharmaceutically acceptable salt,
wherein W is —OH or —SH.

In some embodiments, a double stranded RNAi agent includes:
(i) a sense strand including SEQ ID NO: 907, and
(ii) an antisense strand including SEQ ID NO: 964;
wherein the ligand (L96) is conjugated to 3′ end of the sense strand to form the following schematic:

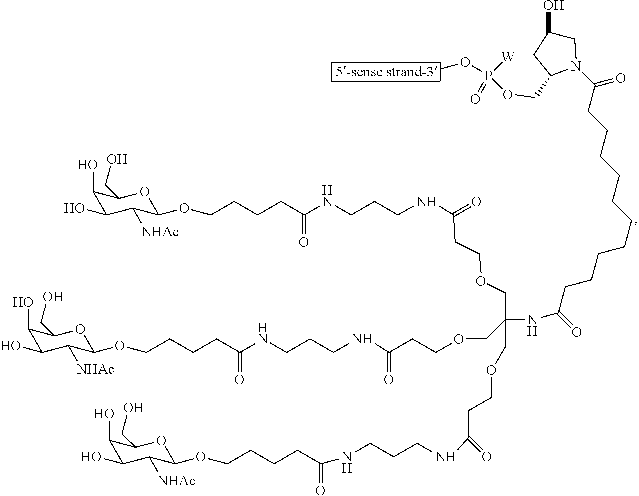

or a pharmaceutically acceptable salt,
wherein W is —OH.

In some embodiments, a dsRNAi agent includes:
(i) a sense strand consisting of SEQ ID NO: 907, and
(ii) an antisense strand consisting of SEQ ID NO: 964;
wherein the ligand (L96) is conjugated to 3′ end of the sense strand to form the following schematic:

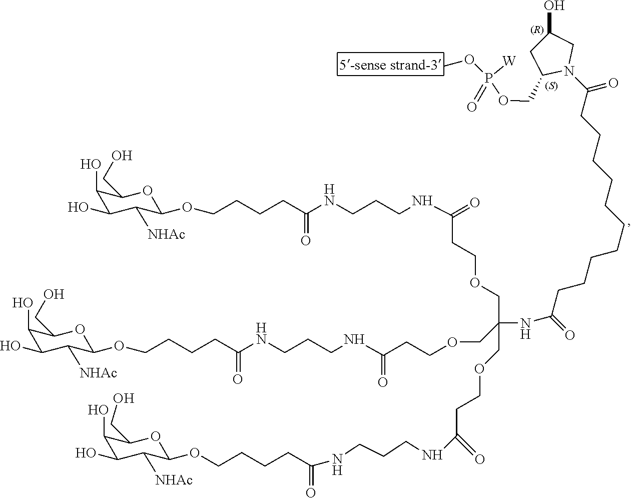

or a pharmaceutically acceptable salt,
wherein W is —OH or —SH.

In some embodiments, a double stranded RNAi agent includes:
(i) a sense strand consisting of SEQ ID NO: 907, and
(ii) an antisense strand consisting of SEQ ID NO: 964;
wherein the ligand (L96) is conjugated to 3′ end of the sense strand to form the following schematic:

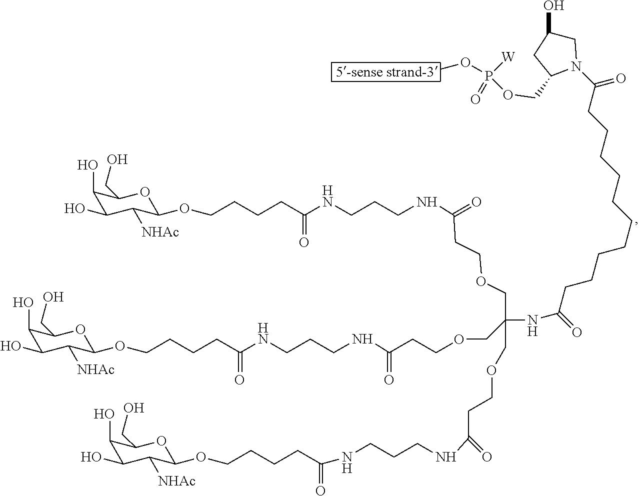

or a pharmaceutically acceptable salt,
wherein W is —OH.

In some embodiments, a dsRNAi agent includes:
(i) a sense strand including SEQ ID NO: 947, and
(ii) an antisense strand including SEQ ID NO: 1004;
wherein the ligand (L96) is conjugated to 3′ end of the sense strand to form the following schematic:

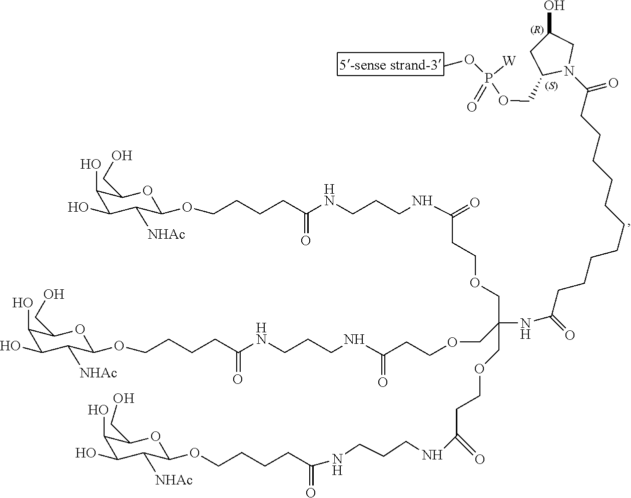

or a pharmaceutically acceptable salt,
wherein W is —OH or —SH.

In some embodiments, a double stranded RNAi agent includes:
(i) a sense strand including SEQ ID NO: 947, and
(ii) an antisense strand including SEQ ID NO: 1004;
wherein the ligand (L96) is conjugated to 3′ end of the sense strand to form the following schematic:

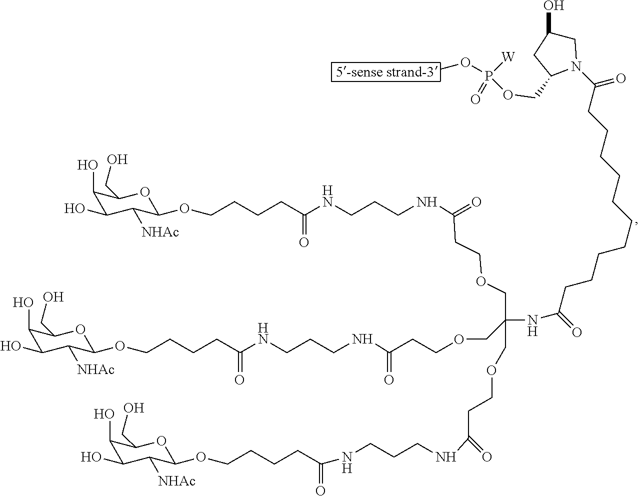

or a pharmaceutically acceptable salt,
wherein W is —OH.

In some embodiments, a dsRNAi agent includes:
(i) a sense strand consisting of SEQ ID NO: 947, and
(ii) an antisense strand consisting of SEQ ID NO: 1004;
wherein the ligand (L96) is conjugated to 3′ end of the sense strand to form the

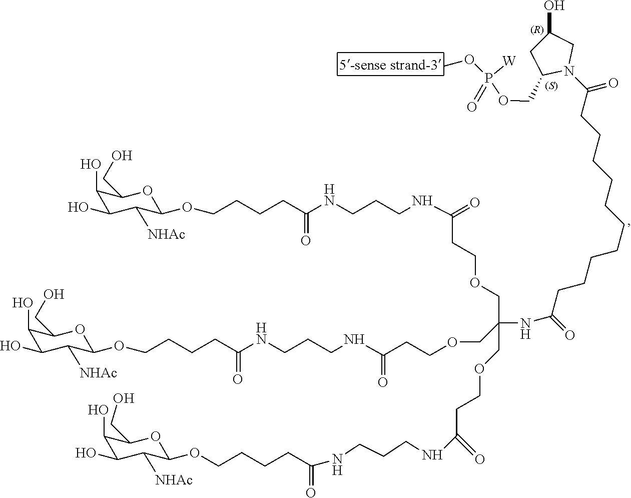

or a pharmaceutically acceptable salt,
wherein W is —OH or —SH.

In some embodiments, a double stranded RNAi agent includes:
(i) a sense strand consisting of SEQ ID NO: 947, and
(ii) an antisense strand consisting of SEQ ID NO: 1004;
wherein the ligand (L96) is conjugated to 3′ end of the sense strand to form the following schematic:

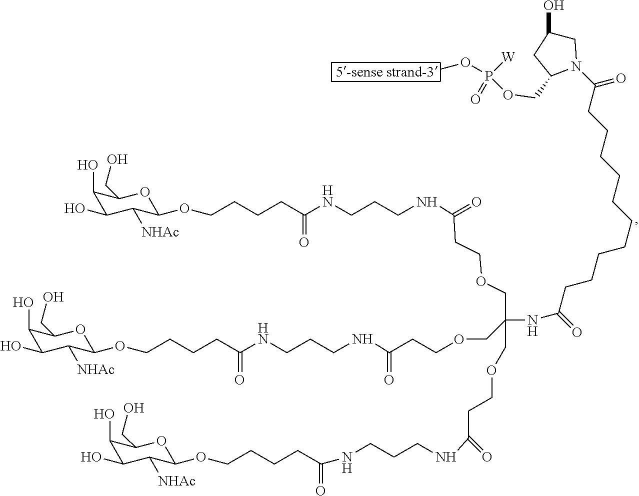

or a pharmaceutically acceptable salt,
wherein W is —OH.

In some embodiments, a dsRNAi agent includes:
(i) a sense strand including SEQ ID NO: 948, and
(ii) an antisense strand including SEQ ID NO: 1005;
wherein the ligand (L96) is conjugated to 3′ end of the sense strand to form the following schematic:

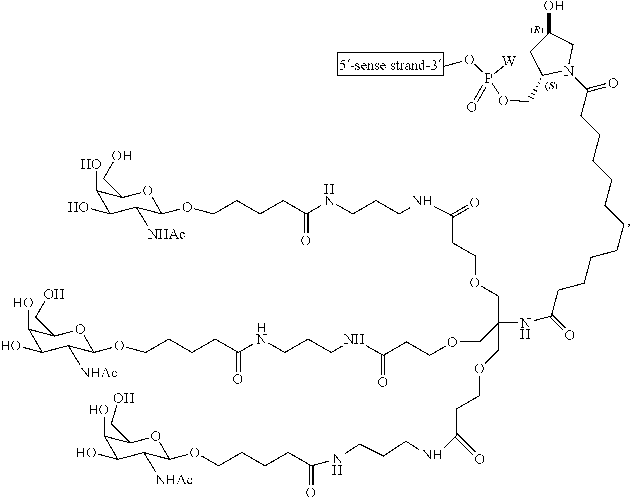

or a pharmaceutically acceptable salt,
wherein W is —OH or —SH.

In some embodiments, a double stranded RNAi agent includes:
(i) a sense strand including SEQ ID NO: 948, and
(ii) an antisense strand including SEQ ID NO: 1005;
wherein the ligand (L96) is conjugated to 3′ end of the sense strand to form the following schematic:

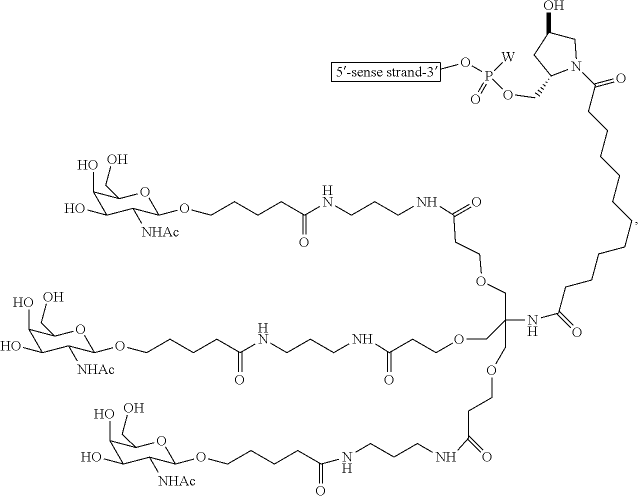

or a pharmaceutically acceptable salt,
wherein W is —OH.

In some embodiments, a dsRNAi agent includes:
(i) a sense strand consisting of SEQ ID NO: 948, and
(ii) an antisense strand consisting of SEQ ID NO: 1005;
wherein the ligand (L96) is conjugated to 3′ end of the sense strand to form the following schematic:

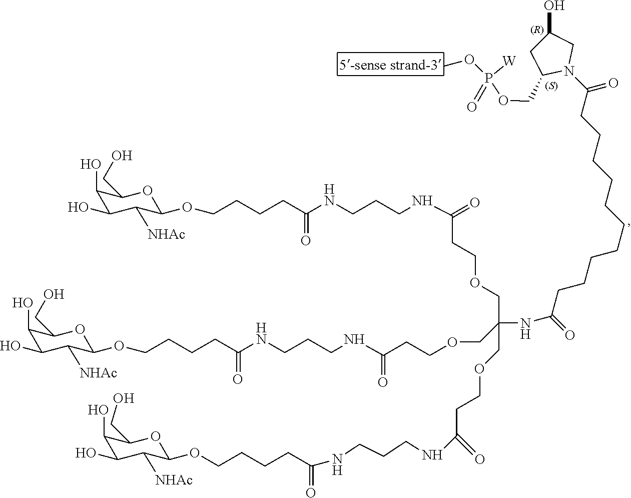

or a pharmaceutically acceptable salt,
wherein W is —OH or —SH.

In some embodiments, a double stranded RNAi agent includes:
(i) a sense strand consisting of SEQ ID NO: 948, and
(ii) an antisense strand consisting of SEQ ID NO: 1005;
wherein the ligand (L96) is conjugated to 3′ end of the sense strand to form the following schematic:

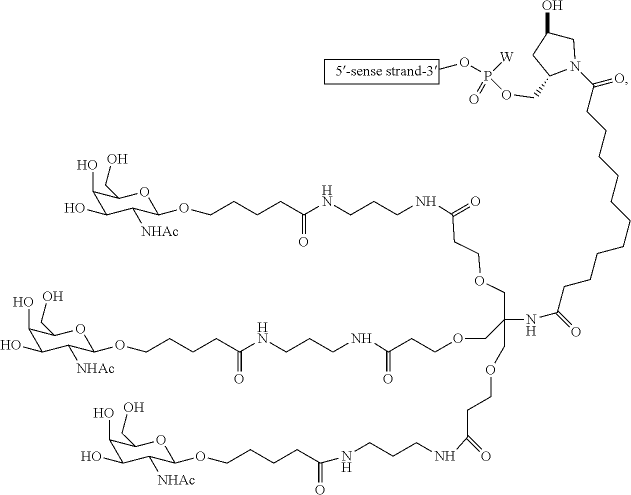

or a pharmaceutically acceptable salt,
wherein W is —OH.

In some embodiments, a dsRNAi agent includes:
(i) a sense strand including SEQ ID NO: 949, and
(ii) an antisense strand including SEQ ID NO: 1006;
wherein the ligand (L96) is conjugated to 3′ end of the sense strand to form the following schematic:

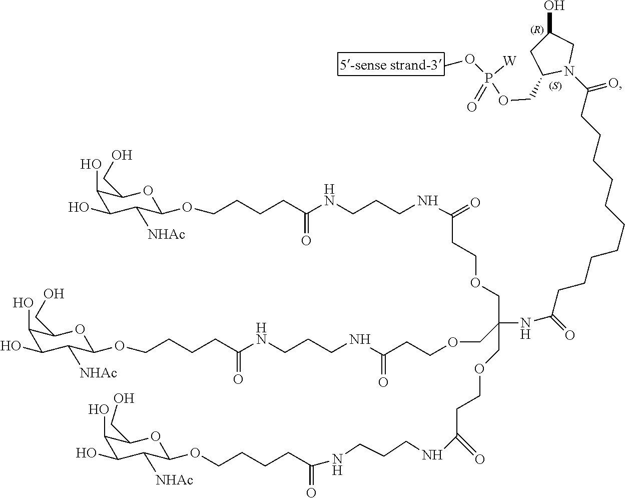

or a pharmaceutically acceptable salt,
wherein W is —OH or —SH.

In some embodiments, a double stranded RNAi agent includes:
(i) a sense strand including SEQ ID NO: 949, and
(ii) an antisense strand including SEQ ID NO: 1006;
wherein the ligand (L96) is conjugated to 3′ end of the sense strand to form the following schematic:

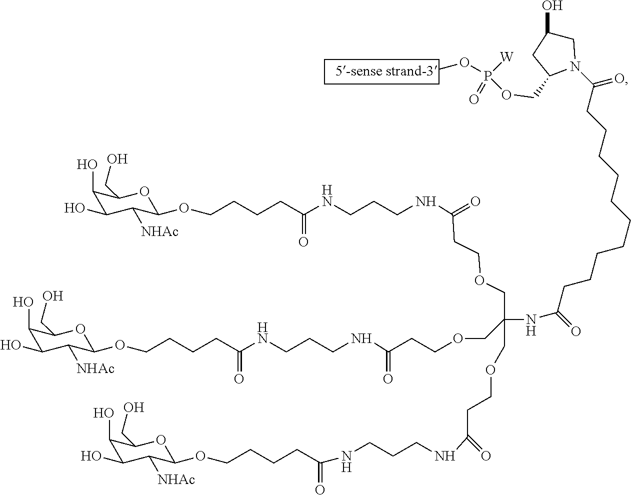

or a pharmaceutically acceptable salt,
wherein W is —OH.

In some embodiments, a dsRNAi agent includes:
(i) a sense strand consisting of SEQ ID NO: 949, and
(ii) an antisense strand consisting of SEQ ID NO: 1006;
wherein the ligand (L96) is conjugated to 3′ end of the sense strand to form the following schematic:

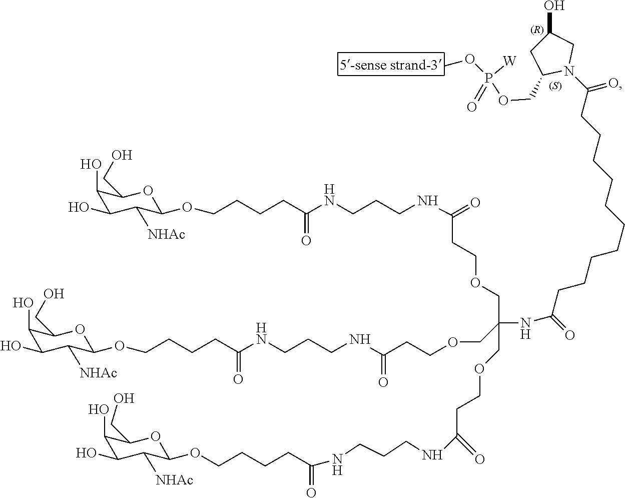

or a pharmaceutically acceptable salt,
wherein W is —OH or —SH.

In some embodiments, a double stranded RNAi agent includes:
(i) a sense strand consisting of SEQ ID NO: 949, and
(ii) an antisense strand consisting of SEQ ID NO: 1006;
wherein the ligand (L96) is conjugated to 3′ end of the sense strand to form the following schematic:

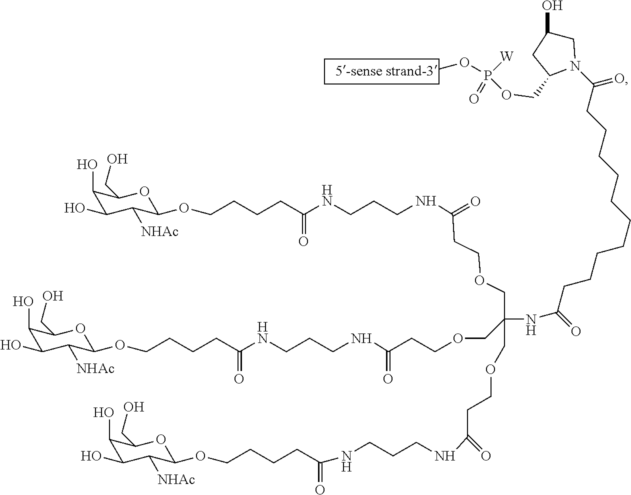

or a pharmaceutically acceptable salt,
wherein W is —OH.

In some embodiments, a dsRNAi agent includes:
(i) a sense strand including SEQ ID NO: 950, and
(ii) an antisense strand including SEQ ID NO: 1007;
wherein the ligand (L96) is conjugated to 3′ end of the sense strand to form the following schematic:

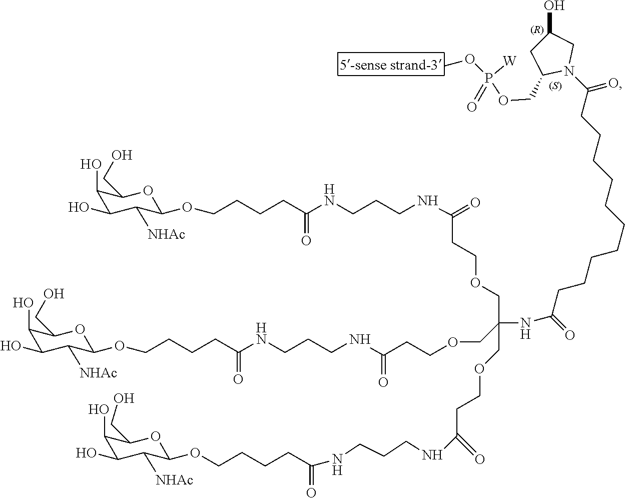

or a pharmaceutically acceptable salt,
wherein W is —OH or —SH.

In some embodiments, a double stranded RNAi agent includes:
(i) a sense strand including SEQ ID NO: 950, and
(ii) an antisense strand including SEQ ID NO: 1007;
wherein the ligand (L96) is conjugated to 3′ end of the sense strand to form the following schematic:

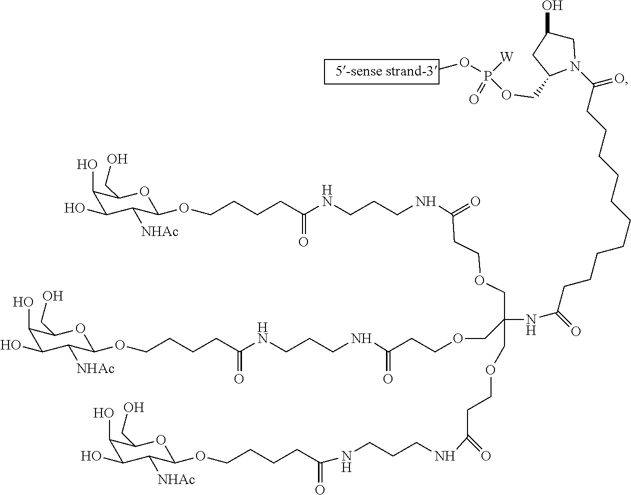

or a pharmaceutically acceptable salt,
wherein W is —OH.

In some embodiments, a dsRNAi agent includes:
(i) a sense strand consisting of SEQ ID NO: 950, and
(ii) an antisense strand consisting of SEQ ID NO: 1007;
wherein the ligand (L96) is conjugated to 3′ end of the sense strand to form the following schematic:

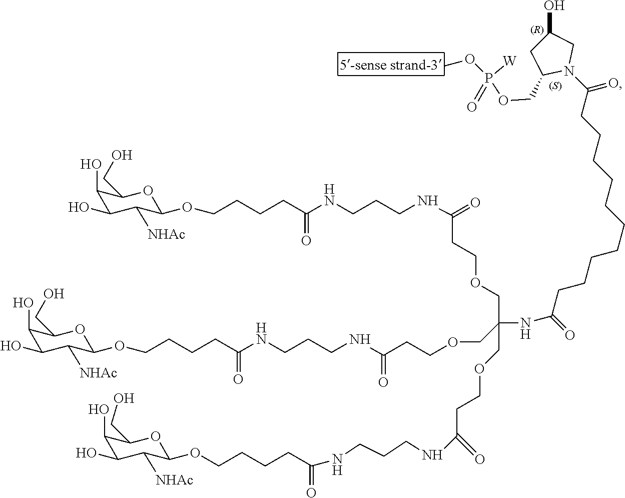

or a pharmaceutically acceptable salt,
wherein W is —OH or —SH.

In some embodiments, a double stranded RNAi agent includes:
(i) a sense strand consisting of SEQ ID NO: 950, and
(ii) an antisense strand consisting of SEQ ID NO: 1007;
wherein the ligand (L96) is conjugated to 3′ end of the sense strand to form the following schematic:

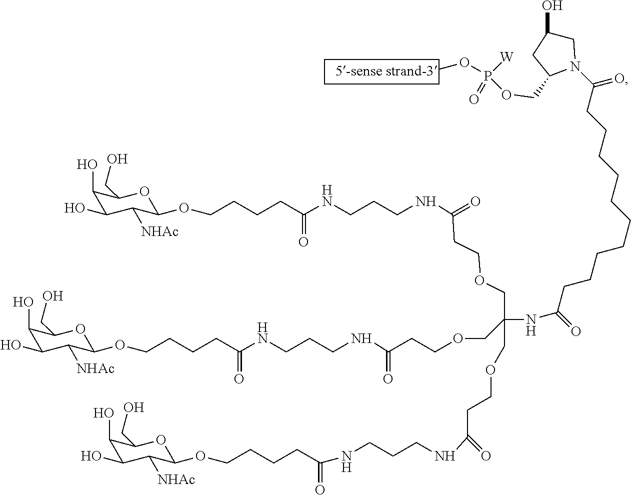

or a pharmaceutically acceptable salt,
wherein W is —OH.

In some embodiments, a double stranded RNAi agent (e.g., siRNA agent) includes:
(i) a sense strand (SS) comprising a nucleotide sequence of

(SEQ ID NO: 800)
5′-C005p001T005p001A004pG004pA004pC004pC007pU
004pG007pU007pT002pU004pU004pG004pC004pU004pU
004pU004pU004pG005pT005-3′;

(ii) an antisense strand (AS) comprising a nucleotide sequence of

(SEQ ID NO: 853)
5′-X033A1027p001C007p001A004pA004pA004pA007pG
004pC004pA004pA004pA004pA004pC004pA007pG004pG
007pU004pC004pU004pA004pG004p001A004p001A004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033A1027 is 5′-(E)-vinylphosphonate-2′-O-methyladenosine; p is a phosphodiester linkage; and p001 is a phosphorothioate linkage,
   wherein the ligand (L96) is

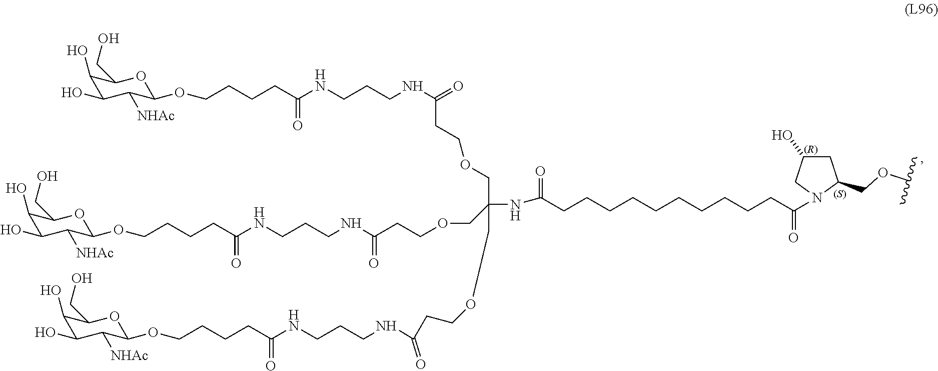

wherein the ligand is conjugated to 3′ end of the sense strand (SS) via a phosphodiester linkage to form the following schematic:

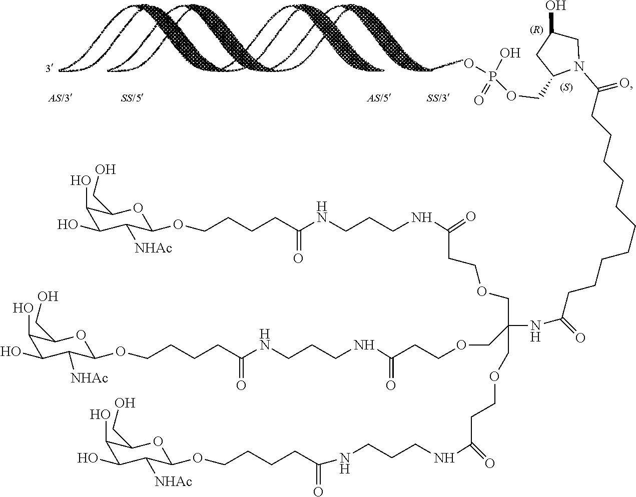

or a pharmaceutically acceptable salt thereof (e.g., sodium salt form).

In some embodiments, a double stranded RNAi agent (e.g., siRNA agent) includes:
(i) a sense strand (SS) consisting of a nucleotide sequence of: 5′-C005p001T005p001A004pG004pA004pC004pC007pU004pG007pU007pT002pU004pU004pG 004pC004pU004pU004pU004pU004pG005pT005-3′ (SEQ ID NO: 800);
(ii) an antisense strand (AS) consisting of a nucleotide sequence of: 5′-X033A1027p001C007p001A004pA004pA004pA007pG004pC004pA004pA004pA004pA004pC 004pA007pG004pG007pU004pC004pU004pA004pG004p001A004p001A004-3′ (SEQ ID NO: 853); and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033A1027 is 5′-(E)-vinylphosphonate-2′-O-methyladenosine; p is a phosphodiester linkage; and p001 is a phosphorothioate linkage,
   wherein the ligand (L96) is

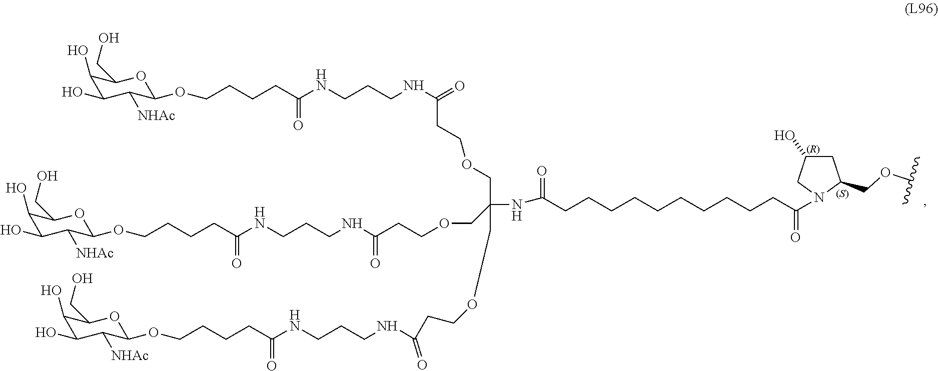

wherein the ligand is conjugated to 3′ end of the sense strand (SS) via a phosphodiester linkage to form the following schematic:

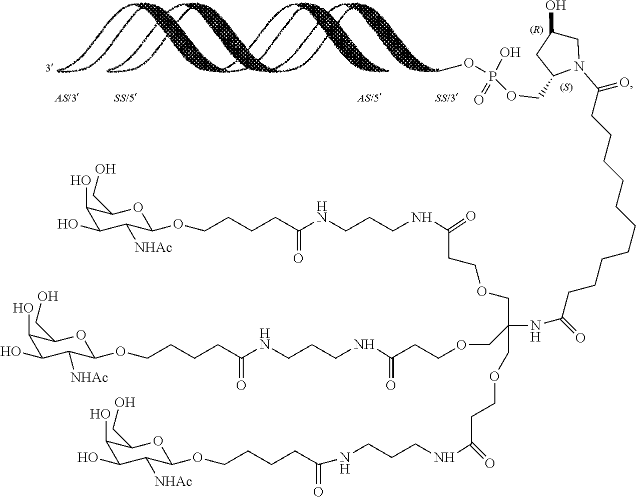

or a pharmaceutically acceptable salt thereof (e.g., sodium salt form).

In some embodiments, a double stranded RNAi agent includes:
(i) a sense strand (SS) comprising

(SEQ ID NO: 906)
5′-C005p001T005p001A004pG004pA004pC004pC007pU
004pG007pU007pT002pU004pU004pG004pC004pU004pU
004pU004pU004pG005pT005pX1085-3′;

and
(ii) an antisense strand (AS) comprising

(SEQ ID NO: 963)
5′-X033A1027p001C007p001A004pA004pA004pA007pG
004pC004pA004pA004pA004pA004pC004pA007pG004pG
007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033A1027 is 5′-(E)-vinylphosphonate-2′-O-methyladenosine; p is a phosphodiester linkage; and p001 is a phosphorothioate linkage, and
X1085 is a ligand (L96),
   wherein the ligand (L96) is

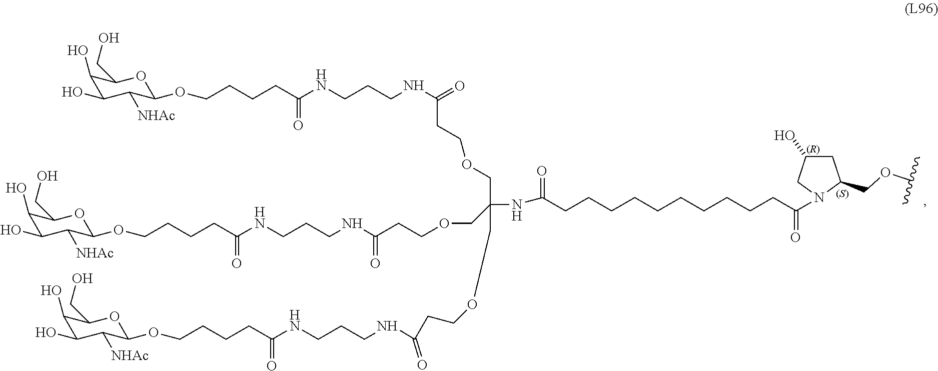

wherein the dsRNAi agent has the following schematic:

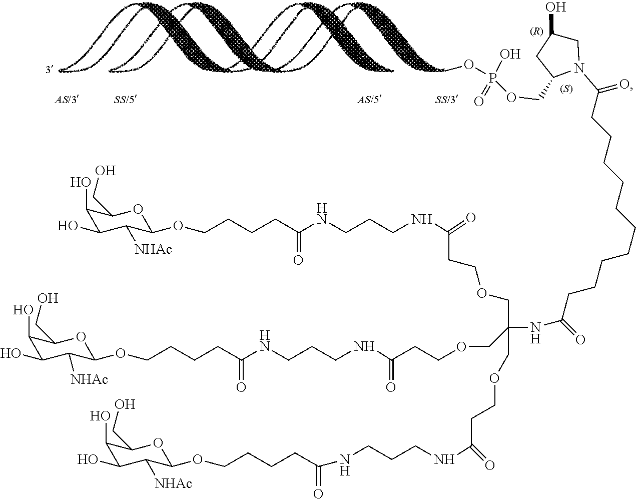

or a pharmaceutically acceptable salt thereof (e.g., sodium salt form).

In some embodiments, a double stranded RNAi agent includes:
(i) a sense strand (SS) consisting of

(SEQ ID NO: 906)
5′-C005p001T005p001A004pG004pA004pC004pC007pU
004pG007pU007pT002pU004pU004pG004pC004pU004pU
004pU004pU004pG005pT005pX1085-3′;

and
(ii) an antisense strand (AS) consisting of

(SEQ ID NO: 963)
5′-X033A1027p001C007p001A004pA004pA004pA007pG
004pC004pA004pA004pA004pA004pC004pA007pG004pG
007pU004pC004pU004pA004pG004p001A004p001A00
4-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033A1027 is 5′-(E)-vinylphosphonate-2′-O-methyladenosine; p is a phosphodiester linkage; and p001 is a phosphorothioate linkage, and
X1085 is a ligand (L96),
   wherein the ligand (L96) is

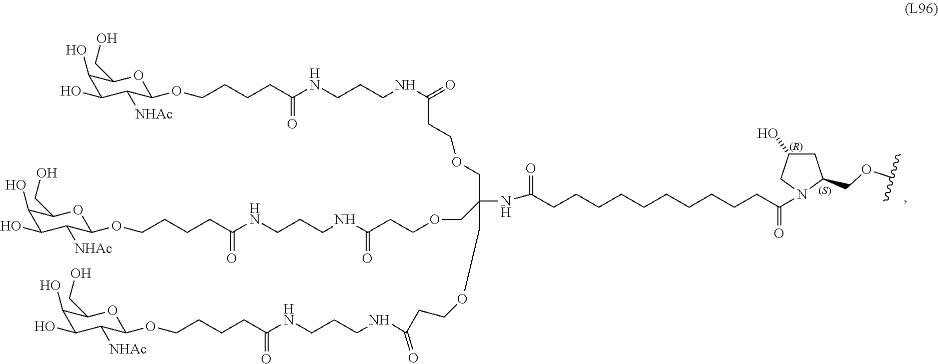

wherein the dsRNAi agent has the following schematic:

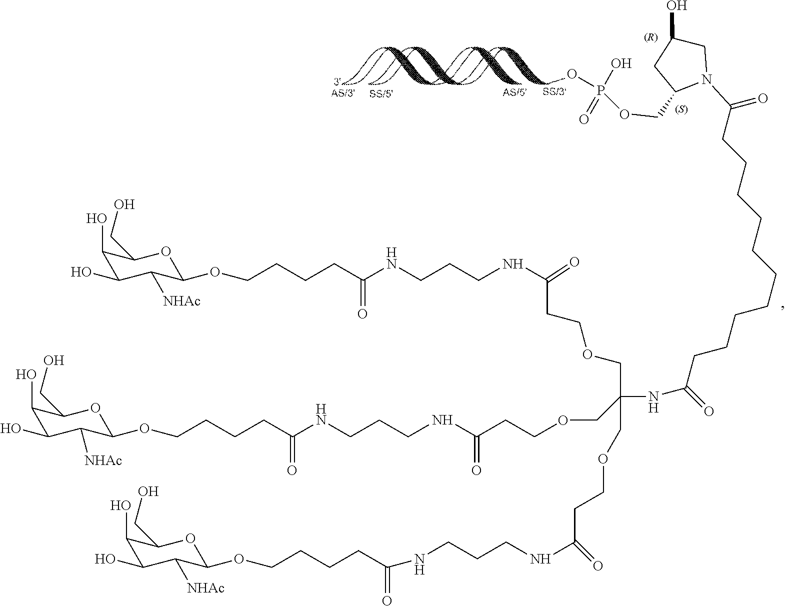

or a pharmaceutically acceptable salt thereof (e.g., sodium salt form).

In some embodiments, a double stranded RNAi agent (e.g., siRNA agent) includes:
(iii) a sense strand (SS) comprising a nucleotide sequence of

(SEQ ID NO: 801)
5′-C005p001T005p001A004pG004pA004pC004pC007pU
004pG007pU007pT002pU004pU004pG004pC004pU004pU
004pU004pU004pG005pT005-3′;

(iv) an antisense strand (AS) comprising a nucleotide sequence of

(SEQ ID NO: 854)
5′-A004p001C007p001A004pA004pA004pA007pG004pC
004pA004pA004pA004pA004pC004pA007pG004pG007pU
004pC004pU004pA004pG004p001A004p001A004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; p is a phosphodiester linkage; and p001 is a phosphorothioate linkage,
   wherein the ligand (L96) is

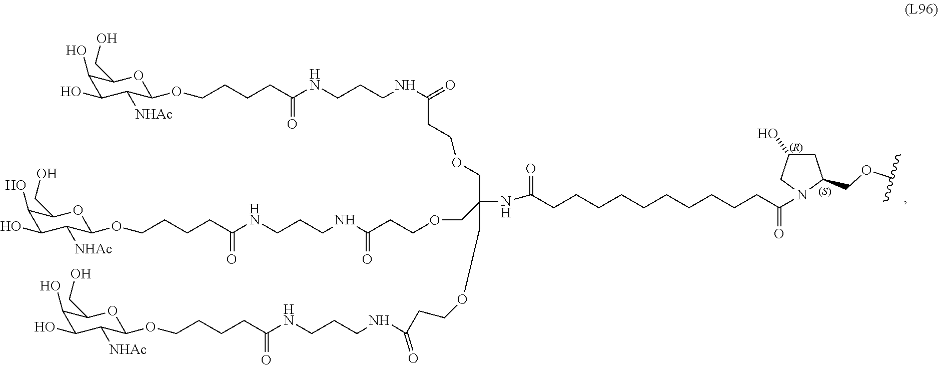

wherein the ligand is conjugated to 3′ end of the sense strand (SS) via a phosphodiester linkage to form the following schematic:

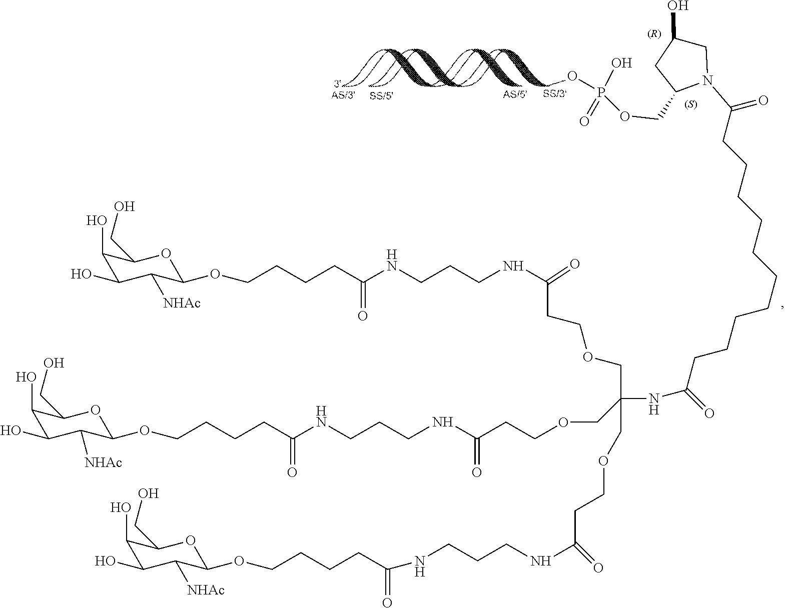

or a pharmaceutically acceptable salt thereof (e.g., sodium salt form).

In some embodiments, a double stranded RNAi agent (e.g., siRNA agent) includes:
(i) a sense strand (SS) consisting of a nucleotide sequence of:

(SEQ ID NO: 801)
5′-C005p001T005p001A004pG004pA004pC004pC007pU
004pG007pU007pT002pU004pU004pG004pC004pU004pU
004pU004pU004pG005pT005-3′;

(ii) an antisense strand (AS) consisting of a nucleotide sequence of:

(SEQ ID NO: 854)
5′-A004p001C007p001A004pA004pA004pA007pG004pC004pA004pA004pA004pA004pC004pA
007pG004pG007pU004pC004pU004pA004pG004p001A004p001A004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; p is a phosphodiester linkage; and p001 is a phosphorothioate linkage,
   wherein the ligand (L96) is

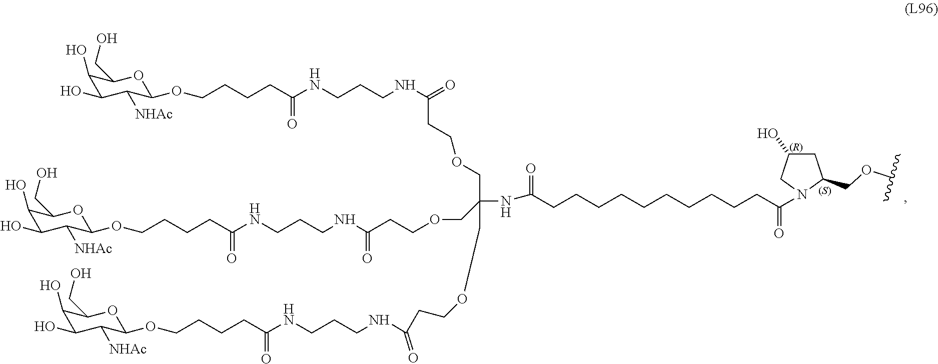

wherein the ligand is conjugated to 3′ end of the sense strand (SS) via a phosphodiester linkage to form the following schematic:

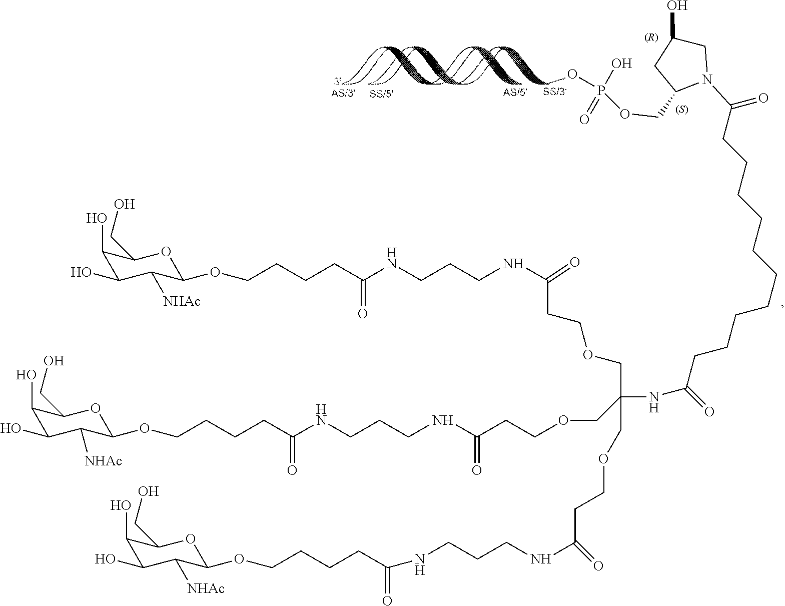

or a pharmaceutically acceptable salt thereof (e.g., sodium salt form).

In some embodiments, a double stranded RNAi agent includes:
(i) a sense strand (SS) comprising

(SEQ ID NO: 907)
5′-C005p001T005p001A004pG004pA004pC004pC007pU004pG007pU007pT002pU004pU004pG
004pC004pU004pU004pU004pU004pG005pT005pX1085-3′;

and
(ii) an antisense strand (AS) comprising

(SEQ ID NO: 964)
5′-A004p001C007p001A004pA004pA004pA007pG004pC004pA004pA004pA004pA004pC004pA
007pG004pG007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; p is a phosphodiester linkage; and p001 is a phosphorothioate linkage, and
X1085 is a ligand (L96),
   wherein the ligand (L96) is

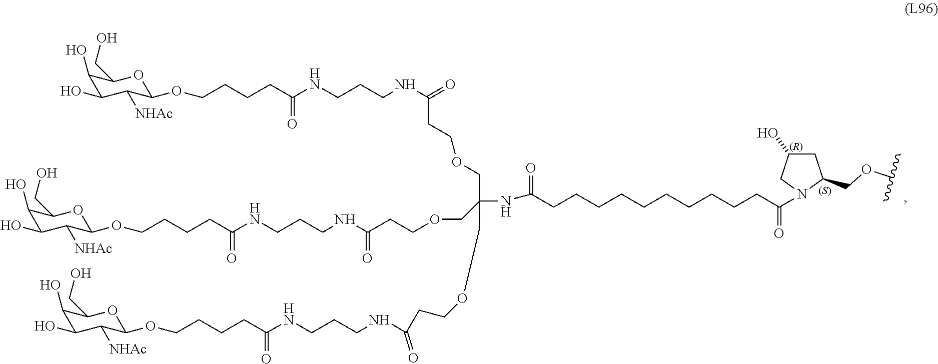

wherein the dsRNAi agent has the following schematic:

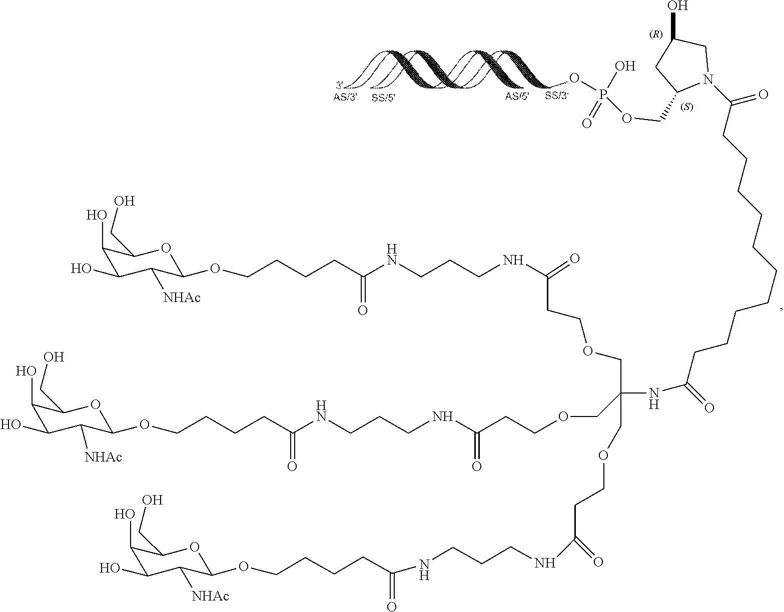

or a pharmaceutically acceptable salt thereof (e.g., sodium salt form).

In some embodiments, a double stranded RNAi agent includes:
(i) a sense strand (SS) consisting of

(SEQ ID NO: 907)
5′-C005p001T005p001A004pG004pA004pC004pC007pU004pG007pU007pT002pU004pU004pG
004pC004pU004pU004pU004pU004pG005pT005pX1085-3′;

and
(ii) an antisense strand (AS) consisting of

(SEQ ID NO: 964)
5′-A004p001C007p001A004pA004pA004pA007pG004pC004pA004pA004pA004pA004pC004pA
007pG004pG007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; p is a phosphodiester linkage; and p001 is a phosphorothioate linkage, and
X1085 is a ligand (L96),
   wherein the ligand (L96) is

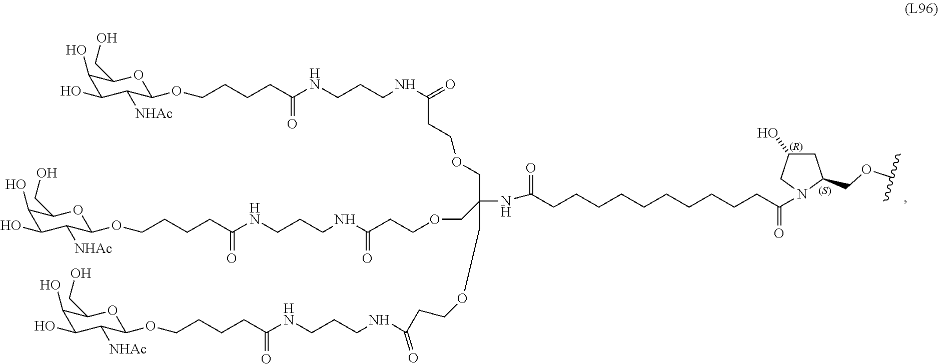

wherein the dsRNAi agent has the following schematic:

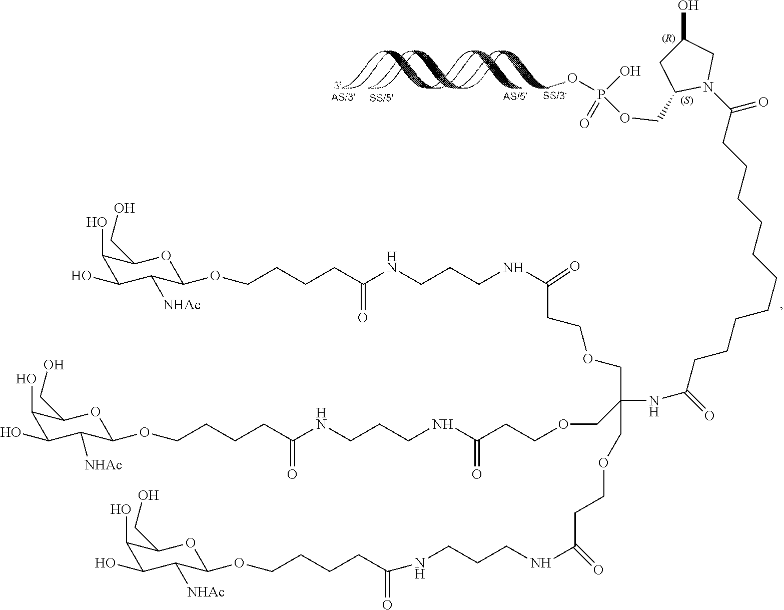

or a pharmaceutically acceptable salt thereof (e.g., sodium salt form).

In some embodiments, a double stranded RNAi agent (e.g., siRNA agent) includes:
(i) a sense strand (SS) comprising a nucleotide sequence of

(SEQ ID NO: 806)
5′-C005p001T005p001A004pG004pA004pC004pC007pU004pG007pU007pT002pU004pU004pG
004pC004pU004pU004pU004pU004p001G005p001T005-3′;

(ii) an antisense strand (AS) comprising a nucleotide sequence of

(SEQ ID NO: 859)
5′-X033A1027p001C007p001A004pA004pA004pA007pG004pC004pA004pA004pA004pA004pC
004pA007pG004pG007pU004pC004pU004pA004pG004p001A004p001A004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033A1027 is 5′-(E)-vinylphosphonate-2′-O-methyladenosine; p is a phosphodiester linkage; and p001 is a phosphorothioate linkage,
   wherein the ligand (L96) is

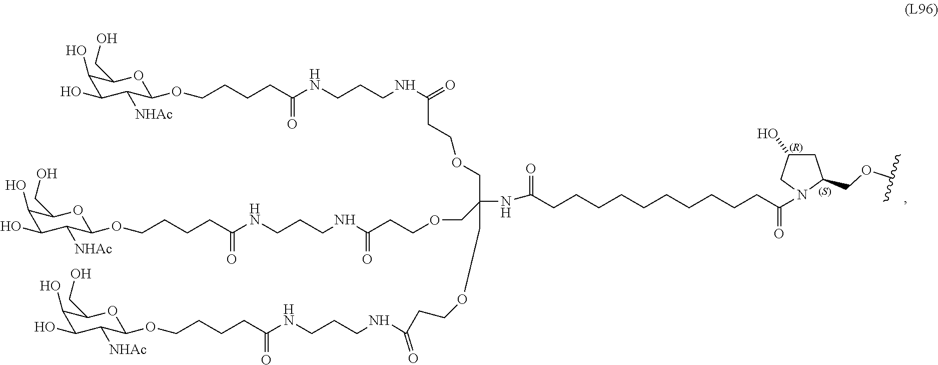

wherein the ligand is conjugated to 3′ end of the sense strand (SS) via a phosphodiester linkage to form the following schematic:

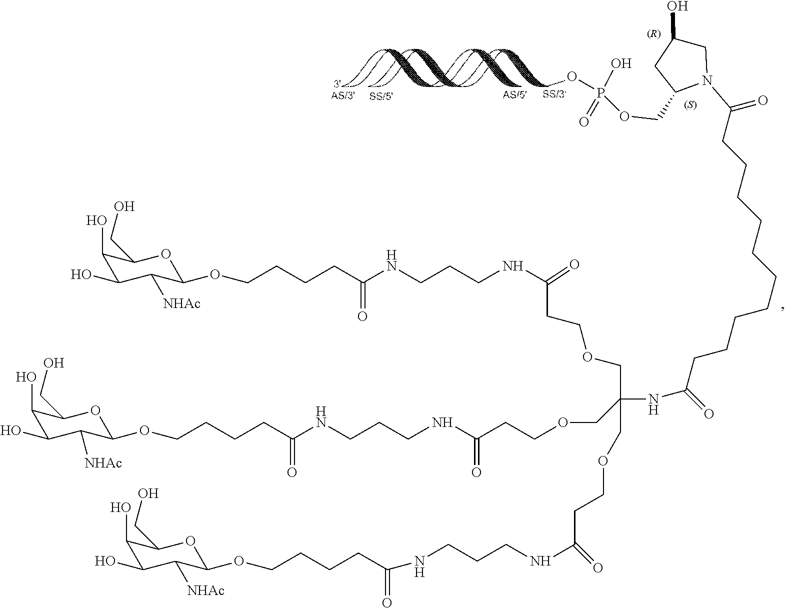

or a pharmaceutically acceptable salt thereof (e.g., sodium salt form).

In some embodiments, a double stranded RNAi agent (e.g., siRNA agent) includes:
(i) a sense strand (SS) consisting of a nucleotide sequence of:

(SEQ ID NO: 806)
5′-C005p001T005p001A004pG004pA004pC004pC007pU004pG007pU007pT002pU004pU004pG
004pC004pU004pU004pU004pU004p001G005p001T005-3′;

(ii) an antisense strand (AS) consisting of a nucleotide sequence of:

(SEQ ID NO: 859)
5′-X033A1027p001C007p001A004pA004pA004pA007pG004pC004pA004pA004pA004pA004pC
004pA007pG004pG007pU004pC004pU004pA004pG004p001A004p001A004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033A1027 is 5′-(E)-vinylphosphonate-2′-O-methyladenosine; p is a phosphodiester linkage; and p001 is a phosphorothioate linkage,
   wherein the ligand (L96) is

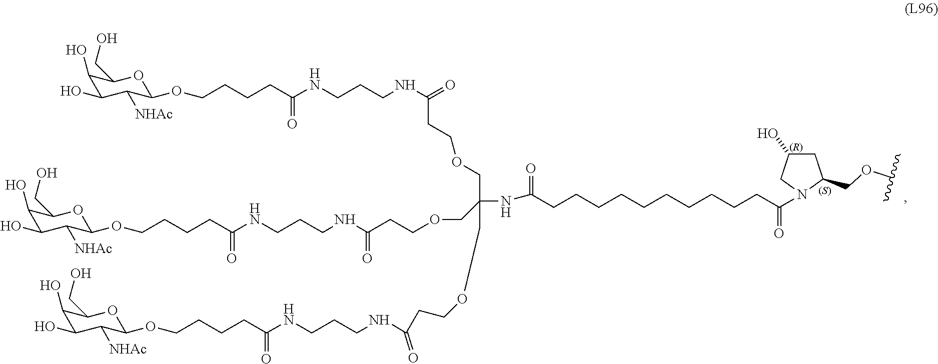

wherein the ligand is conjugated to 3′ end of the sense strand (SS) via a phosphodiester linkage to form the following schematic:

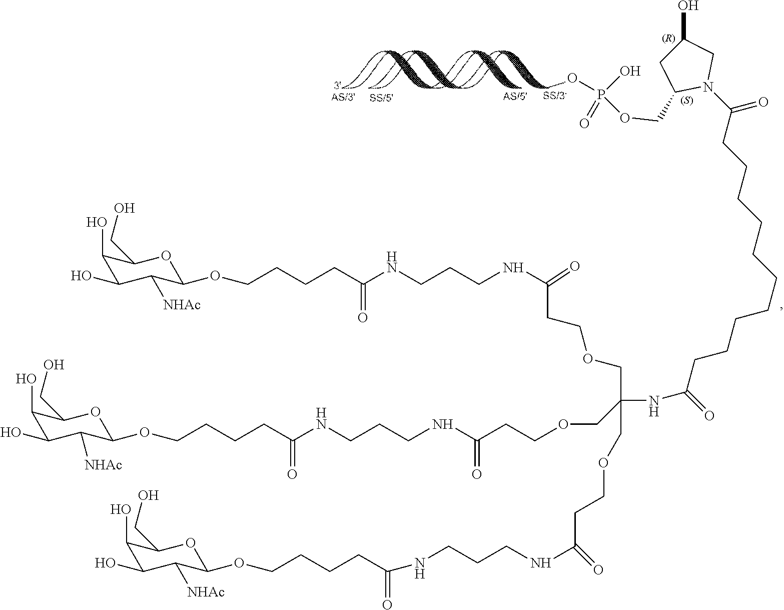

or a pharmaceutically acceptable salt thereof (e.g., sodium salt form).

In some embodiments, a double stranded RNAi agent includes:
(i) a sense strand (SS) comprising

(SEQ ID NO: 947)
5′-C005p001T005p001A004pG004pA004pC004pC007pU004pG007pU007pT002pU004pU004pG
004pC004pU004pU004pU004pU004p001G005p001T005pX1085-3′;

and
(ii) an antisense strand (AS) comprising

(SEQ ID NO: 1004)
5′-X033A1027p001C007p001A004pA004pA004pA007pG004pC004pA004pA004pA004pA004pC
004pA007pG004pG007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033A1027 is 5′-(E)-vinylphosphonate-2′-O-methyladenosine; p is a phosphodiester linkage; and p001 is a phosphorothioate linkage, and
X1085 is a ligand (L96),
   wherein the ligand (L96) is

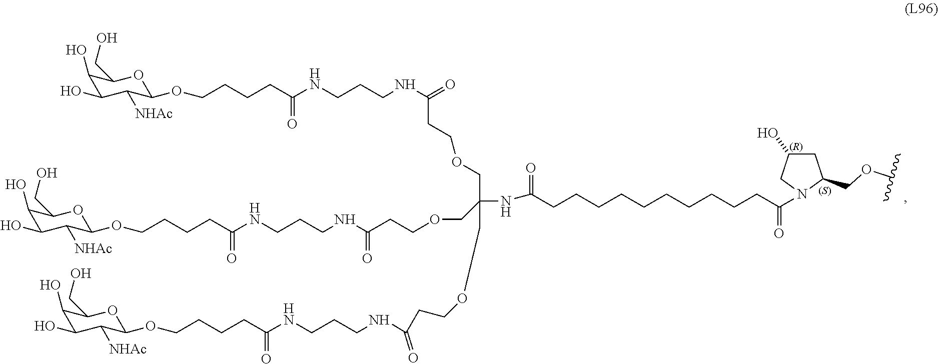

wherein the dsRNAi agent has the following schematic:

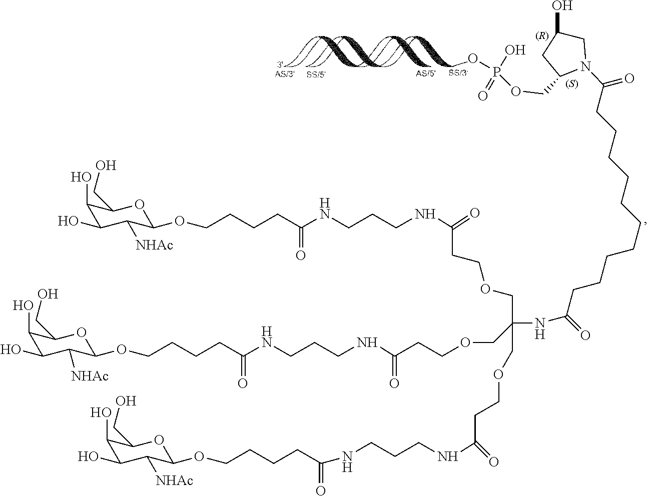

or a pharmaceutically acceptable salt thereof (e.g., sodium salt form).

In some embodiments, a double stranded RNAi agent includes:
(i) a sense strand (SS) consisting of

(SEQ ID NO: 947)
5′-C005p001T005p001A004pG004pA004pC004pC007pU004pG007pU007pT002pU004pU004pG
004pC004pU004pU004pU004pU004p001G005p001T005pX1085-3′;

and
(ii) an antisense strand (AS) consisting of

(SEQ ID NO: 1004)
5′-X033A1027p001C007p001A004pA004pA004pA007pG004pC004pA004pA004pA004pA004pC
004pA007pG004pG007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033A1027 is 5′-(E)-vinylphosphonate-2′-O-methyladenosine; p is a phosphodiester linkage; and p001 is a phosphorothioate linkage, and
X1085 is a ligand (L96),
   wherein the ligand (L96) is

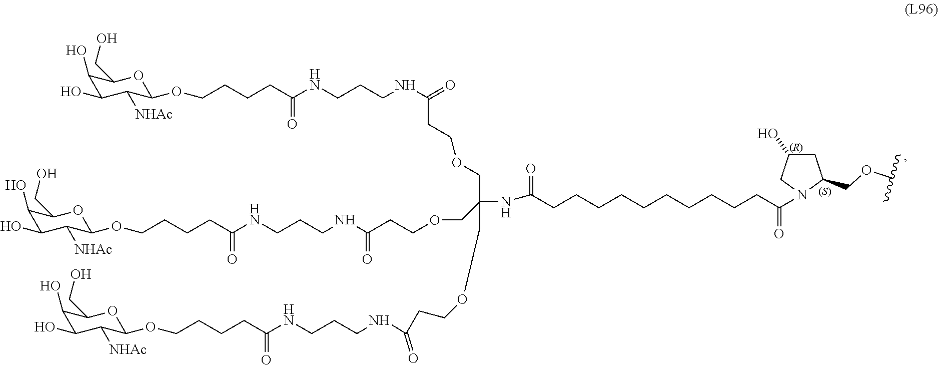

wherein the dsRNAi agent has the following schematic:

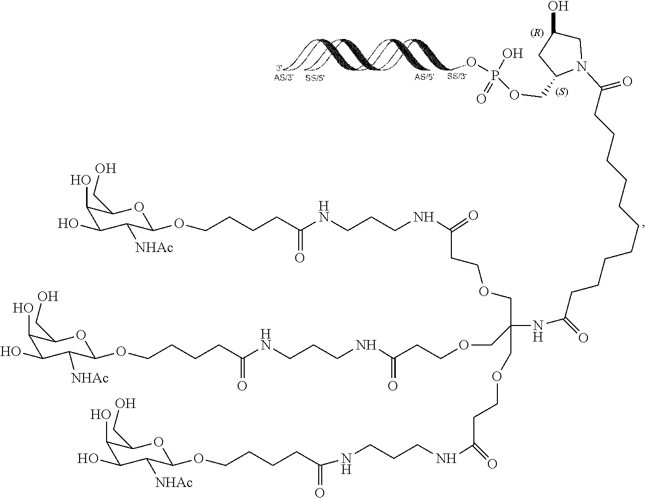

or a pharmaceutically acceptable salt thereof (e.g., sodium salt form).

In some embodiments, a double stranded RNAi agent (e.g., siRNA agent) includes:
(i) a sense strand (SS) comprising a nucleotide sequence of

(SEQ ID NO: 811)
5′-C005p001T005p001A004pG004pA004pC004pC007pU004pG007pU007pT002pU004pU004pG
004pC004pU004pU004pU004pU004p001G005p001T005-3′;

(ii) an antisense strand (AS) comprising a nucleotide sequence of

(SEQ ID NO: 864)
5′-X033A1027p001C007p001A042pA004pA004pA007pG004pC004pA004pA004pA004pA004pC
004pA007pG004pG007pU004pC004pU004pA004pG004p001A004p001A004-3′;

and
a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; X033A1027 is 5′-(E)-vinylphosphonate-2′-O-methyladenosine; p is a phosphodiester linkage; and p001 is a phosphorothioate linkage,
   wherein the ligand (L96) is

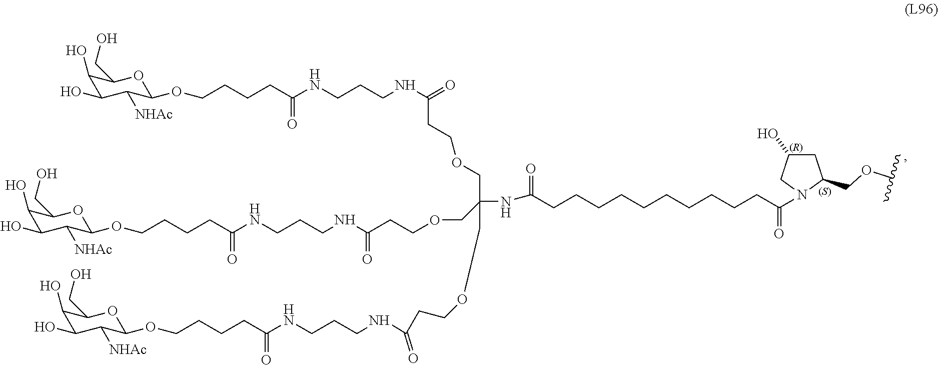

wherein the ligand is conjugated to 3′ end of the sense strand (SS) via a phosphodiester linkage to form the following schematic:

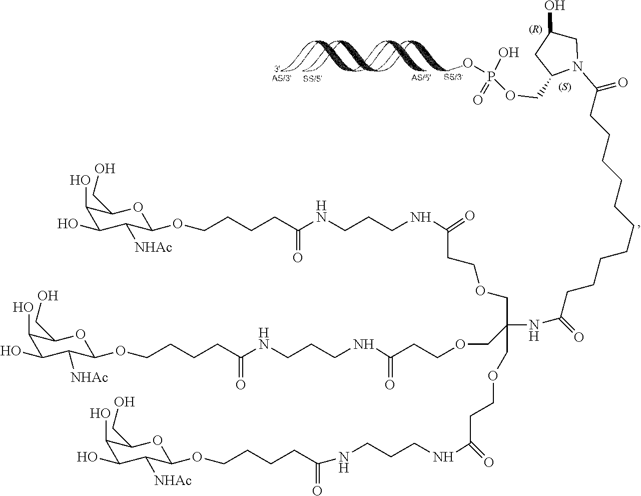

or a pharmaceutically acceptable salt thereof (e.g., sodium salt form).

In some embodiments, a double stranded RNAi agent (e.g., siRNA agent) includes:
(i) a sense strand (SS) consisting of a nucleotide sequence of:

(SEQ ID NO: 811)
5′-C005p001T005p001A004pG004pA004pC004pC007pU004pG007pU007pT002pU004pU004pG
004pC004pU004pU004pU004pU004p001G005p001T005-3′;

(ii) an antisense strand (AS) consisting of a nucleotide sequence of:

(SEQ ID NO: 864)
5′-X033A1027p001C007p001A042pA004pA004pA007pG004pC004pA004pA004pA004pA004pC
004pA007pG004pG007pU004pC004pU004pA004pG004p001A004p001A004-3′;

and
a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; X033A1027 is 5′-(E)-vinylphosphonate-2′-O-methyladenosine; p is a phosphodiester linkage; and p001 is a phosphorothioate linkage,
   wherein the ligand (L96) is

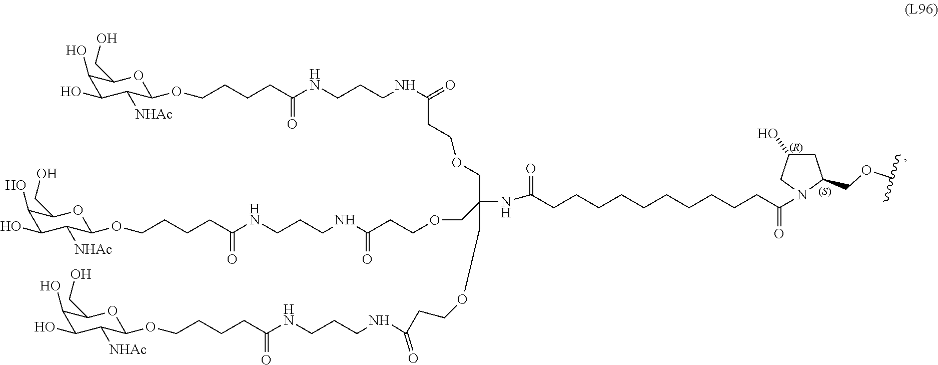

wherein the ligand is conjugated to 3′ end of the sense strand (SS) via a phosphodiester linkage to form the following schematic:

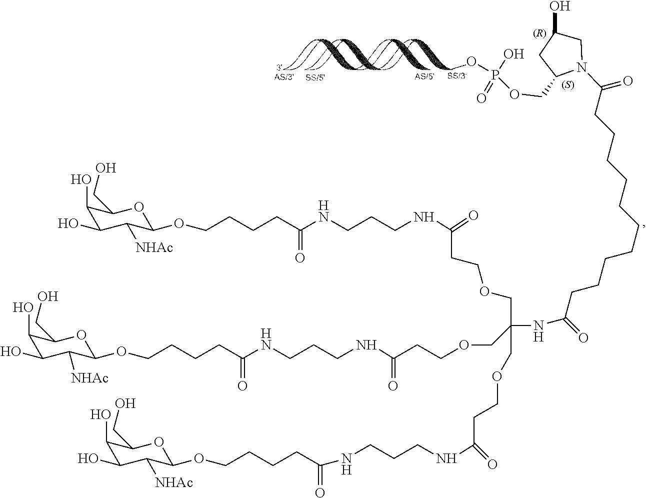

or a pharmaceutically acceptable salt thereof (e.g., sodium salt form).

In some embodiments, a double stranded RNAi agent includes:
(i) a sense strand (SS) comprising

(SEQ ID NO: 948)
5′-C005p001T005p001A004pG004pA004pC004pC007pU004pG007pU007pT002pU004pU004pG
004pC004pU004pU004pU004pU004p001G005p001T005pX1085-3′;

and
(ii) an antisense strand (AS) comprising

(SEQ ID NO: 1005)
5′-X033A1027p001C007p001A042pA004pA004pA007pG004pC004pA004pA004pA004pA004pC
004pA007pG004pG007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; X033A1027 is 5′-(E)-vinylphosphonate-2′-O-methyladenosine; p is a phosphodiester linkage; and p001 is a phosphorothioate linkage, and
X1085 is a ligand (L96),
   wherein the ligand (L96) is

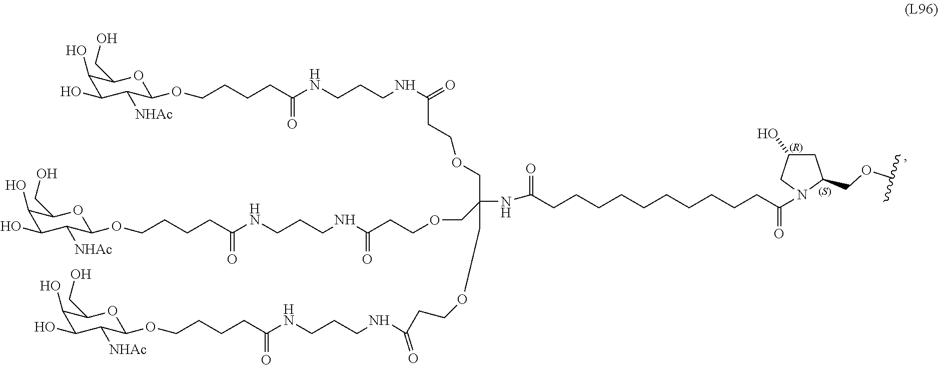

wherein the dsRNAi agent has the following schematic:

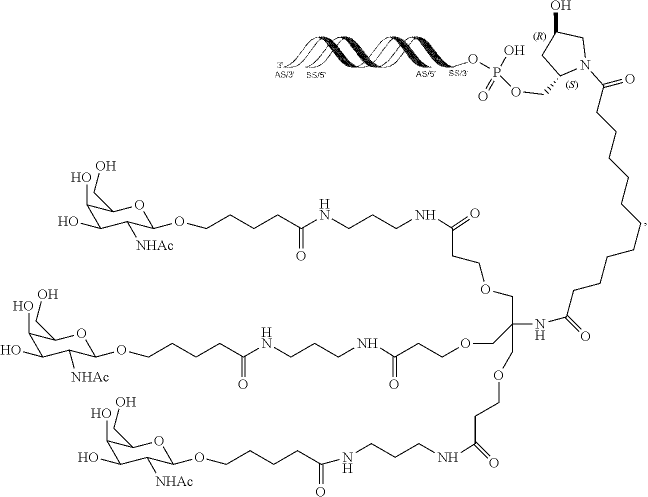

or a pharmaceutically acceptable salt thereof (e.g., sodium salt form).

In some embodiments, a double stranded RNAi agent includes:
(i) a sense strand (SS) consisting of

(SEQ ID NO: 948)
5′-C005p001T005p001A004pG004pA004pC004pC007pU004pG007pU007pT002pU004pU004pG
004pC004pU004pU004pU004pU004p001G005p001T005pX1085-3′;

and
(ii) an antisense strand (AS) consisting of

(SEQ ID NO: 1005)
5′-X033A1027p001C007p001A042pA004pA004pA007pG004pC004pA004pA004pA004pA004pC
004pA007pG004pG007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; X033A1027 is 5′-(E)-vinylphosphonate-2′-O-methyladenosine; p is a phosphodiester linkage; and p001 is a phosphorothioate linkage, and
X1085 is a ligand (L96),
   wherein the ligand (L96) is

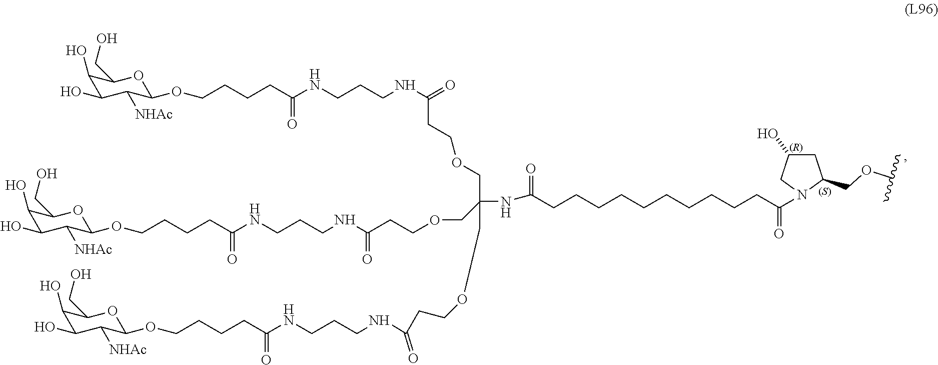

wherein the dsRNAi agent has the following schematic:

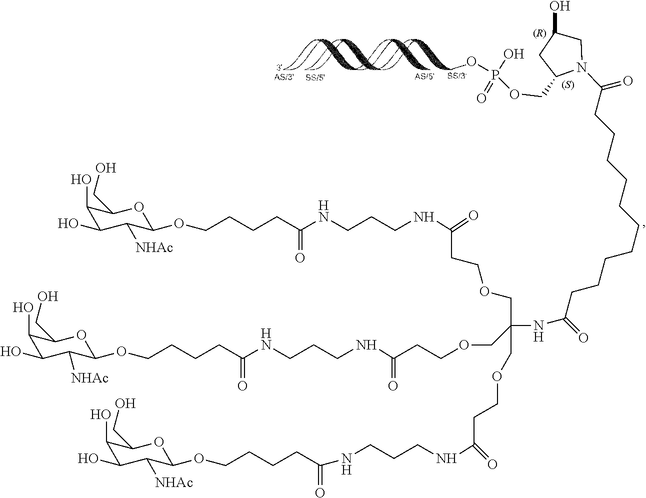

or a pharmaceutically acceptable salt thereof (e.g., sodium salt form).

In some embodiments, a double stranded RNAi agent (e.g., siRNA agent) includes:
(i) a sense strand (SS) comprising a nucleotide sequence of

(SEQ ID NO: 813)
5′-C042p001U042p001A004pG004pA004pC004pC007pU004pG007pU007pT002pU004pU004pG
004pC004pU004pU004pU004pU004p001G042p001U042-3′;

(ii) an antisense strand (AS) comprising a nucleotide sequence of

(SEQ ID NO: 866)
5′-X033A1027p001C007p001A004pA004pA004pA007pG004pC004pA004pA004pA004pA004pC
004pA007pG004pG007pU004pC004pU004pA004pG004p001A004p001A004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; U042 is uridine TNA; C042 is cytidine TNA; G042 is guanosine TNA; X033A1027 is 5′-(E)-vinylphosphonate-2′-O-methyladenosine; p is a phosphodiester linkage; and p001 is a phosphorothioate linkage,
   wherein the ligand (L96) is

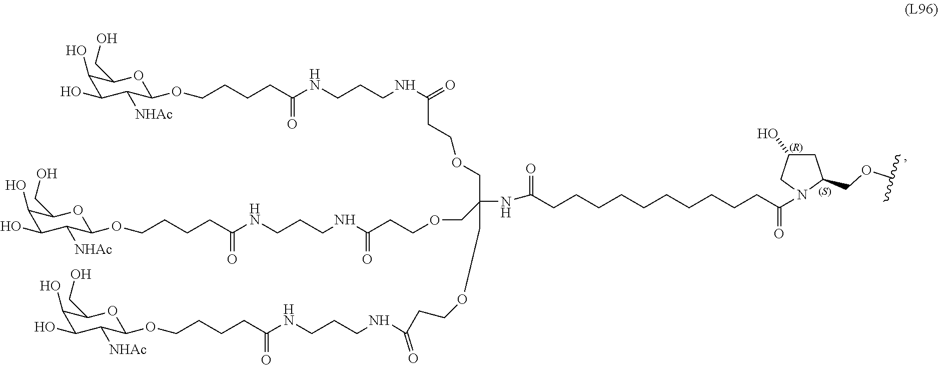

wherein the ligand is conjugated to 3′ end of the sense strand (SS) via a phosphodiester linkage to form the following schematic:

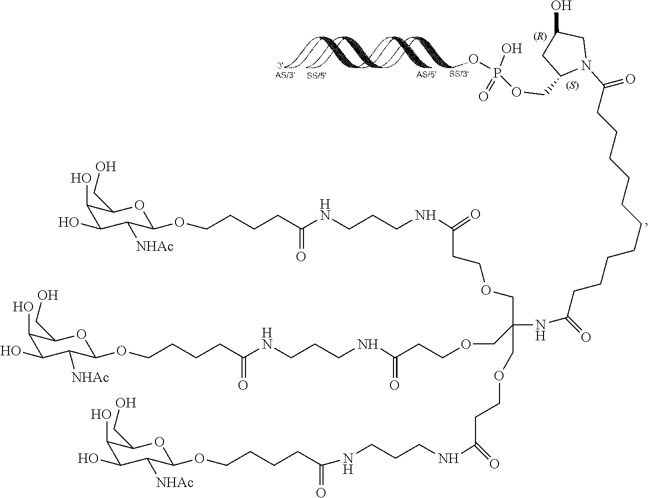

or a pharmaceutically acceptable salt thereof (e.g., sodium salt form).

In some embodiments, a double stranded RNAi agent (e.g., siRNA agent) includes:
(i) a sense strand (SS) consisting of a nucleotide sequence of:

(SEQ ID NO: 813)
5′-C042p001U042p001A004pG004pA004pC004pC007pU004pG007pU007pT002pU004pU004pG
004pC004pU004pU004pU004pU004p001G042p001U042-3′;

(ii) an antisense strand (AS) consisting of a nucleotide sequence of:

(SEQ ID NO: 866)
5′-X033A1027p001C007p001A004pA004pA004pA007pG004pC004pA004pA004pA004pA004pC
004pA007pG004pG007pU004pC004pU004pA004pG004p001A004p001A004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; U042 is uridine TNA; C042 is cytidine TNA; G042 is guanosine TNA; X033A1027 is 5′-(E)-vinylphosphonate-2′-O-methyladenosine; p is a phosphodiester linkage; and p001 is a phosphorothioate linkage, wherein the ligand (L96) is

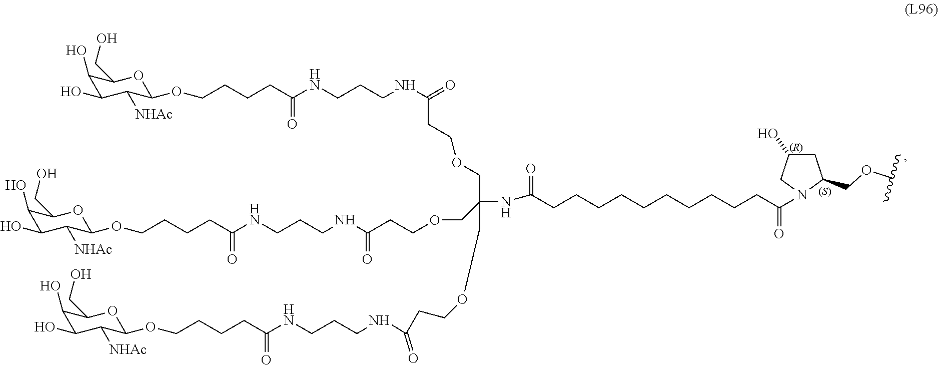

wherein the ligand is conjugated to 3′ end of the sense strand (SS) via a phosphodiester linkage to form the following schematic:

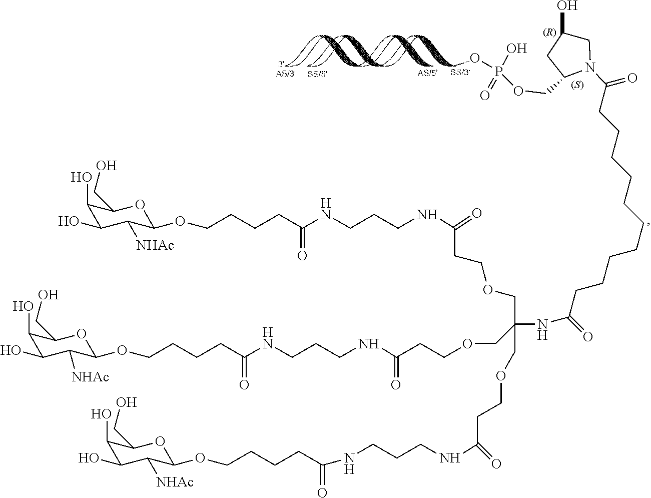

or a pharmaceutically acceptable salt thereof (e.g., sodium salt form).

In some embodiments, a double stranded RNAi agent includes:
(i) a sense strand (SS) comprising

(SEQ ID NO: 949)
5′-C042p001U042p001A004pG004pA004pC004pC007pU004pG007pU007pT002pU004pU004pG
004pC004pU004pU004pU004pU004p001G042p001U042pX1085-3′;

and
(ii) an antisense strand (AS) comprising

(SEQ ID NO: 1006)
5′-X033A1027p001C007p001A004pA004pA004pA007pG004pC004pA004pA004pA004pA004pC
004pA007pG004pG007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; U042 is uridine TNA; C042 is cytidine TNA; G042 is guanosine TNA; X033A1027 is 5′-(E)-vinylphosphonate-2′-O-methyladenosine; p is a phosphodiester linkage; and p001 is a phosphorothioate linkage, and
X1085 is a ligand (L96),
   wherein the ligand (L96) is

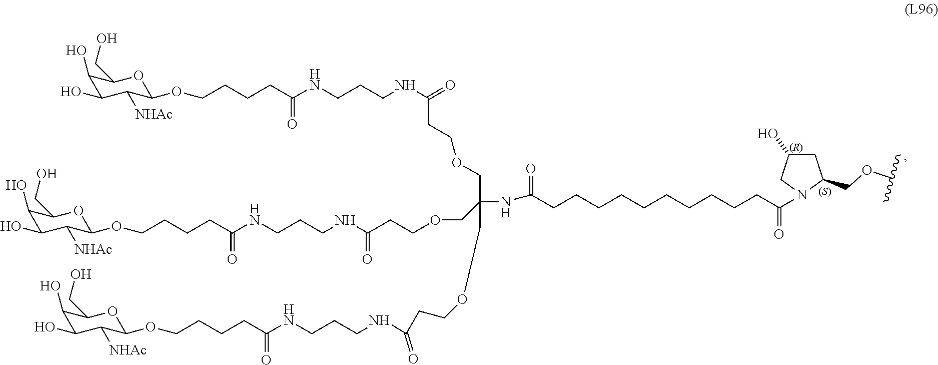

wherein the dsRNAi agent has the following schematic:

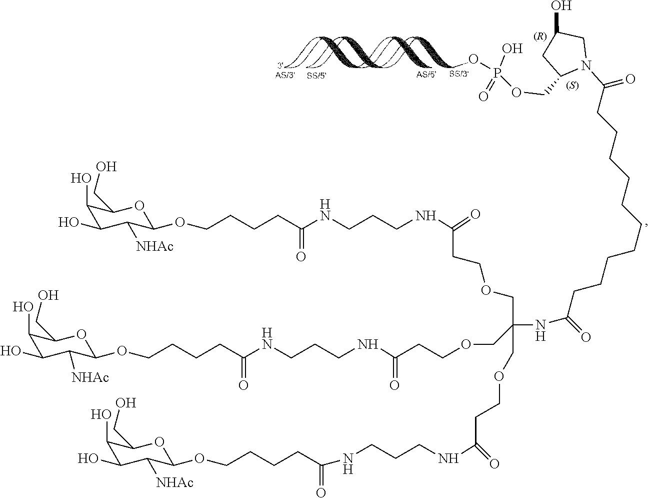

or a pharmaceutically acceptable salt thereof (e.g., sodium salt form).

In some embodiments, a double stranded RNAi agent includes:
(i) a sense strand (SS) consisting of

(SEQ ID NO: 949)
5′-C042p001U042p001A004pG004pA004pC004pC007pU004pG007pU007pT002pU004pU004pG
004pC004pU004pU004pU004pU004p001G042p001U042pX1085-3′;

and
(ii) an antisense strand (AS) consisting of

(SEQ ID NO: 1006)
5′-X033A1027p001C007p001A004pA004pA004pA007pG004pC004pA004pA004pA004pA004pC
004pA007pG004pG007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; U042 is uridine TNA; C042 is cytidine TNA; G042 is guanosine TNA; X033A1027 is 5′-(E)-vinylphosphonate-2′-O-methyladenosine; p is a phosphodiester linkage; and p001 is a phosphorothioate linkage, and
X1085 is a ligand (L96),
   wherein the ligand (L96) is

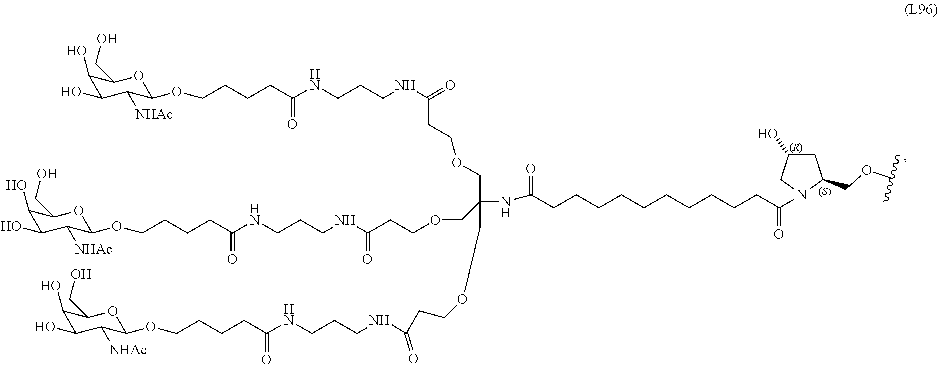

wherein the dsRNAi agent has the following schematic:

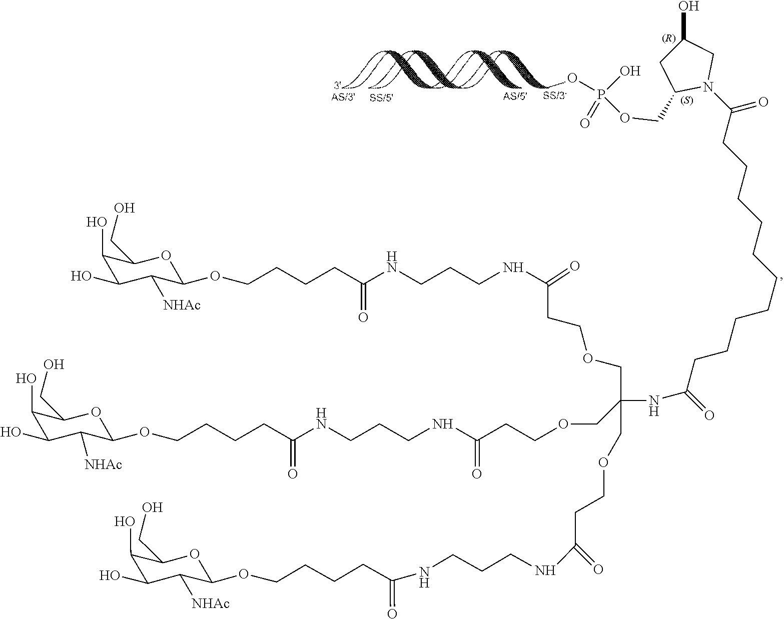

or a pharmaceutically acceptable salt thereof (e.g., sodium salt form).

In some embodiments, a double stranded RNAi agent (e.g., siRNA agent) includes:
(i) a sense strand (SS) comprising a nucleotide sequence of

(SEQ ID NO: 830)
5′-G005p001T005p001U004pU004pU004pG004pC007pU004pU007pU007pU007pG004pU004pA
004pA004pC004pU004pU004pG004p001A005p001A005-3′;

(ii) an antisense strand (AS) comprising a nucleotide sequence of

(SEQ ID NO: 883)
5′-X033U1027p001U007p001C004pA004pA004pG007pU004pU004pA004pC004pA004pA004
pA004pA007pG004pC007pA004pA004pA004pA004pC004p001A004p001G004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
   wherein the ligand (L96) is

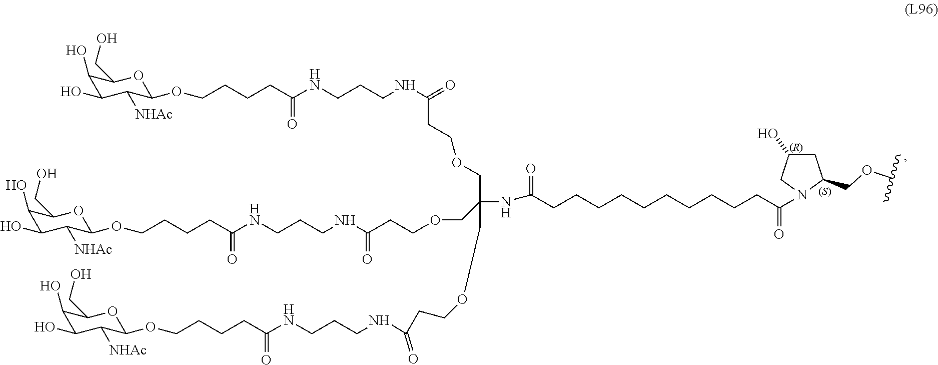

wherein the ligand is conjugated to 3′ end of the sense strand (SS) via a phosphodiester linkage to form the following schematic:

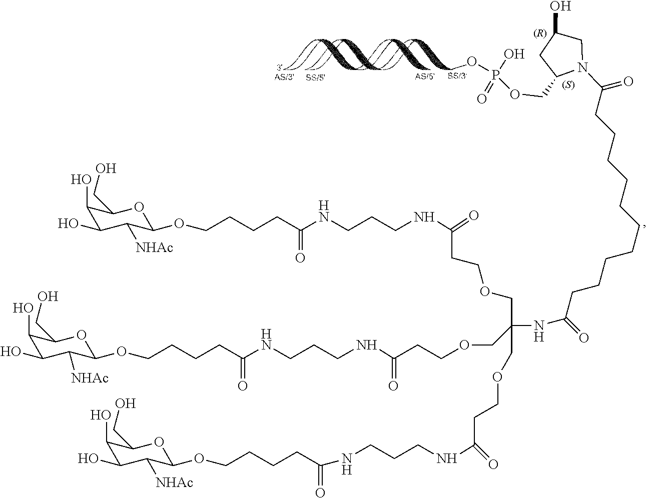

or a pharmaceutically acceptable salt thereof (e.g., sodium salt form).

In some embodiments, a double stranded RNAi agent (e.g., siRNA agent) includes:
(i) a sense strand (SS) consisting of a nucleotide sequence of:

(SEQ ID NO: 830)
5′-G005p001T005p001U004pU004pU004pG004pC007pU004pU007pU007pU007pG004pU004
pA004pA004pC004pU004pU004pG004p001A005p001A005-3′;

(ii) an antisense strand (AS) consisting of a nucleotide sequence of:

(SEQ ID NO: 883)
5′-X033U1027p001U007p001C004pA004pA004pG007pU004pU004pA004pC004pA004pA004pA
004pA007pG004pC007pA004pA004pA004pA004pC004p001A004p001G004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
   wherein the ligand (L96) is

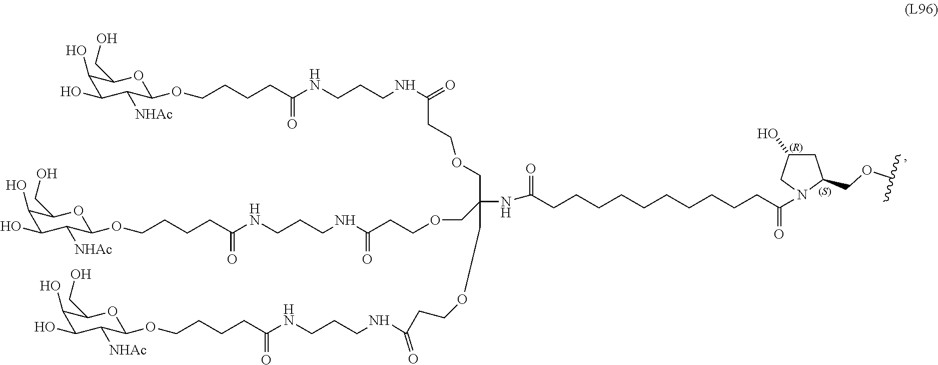

wherein the ligand is conjugated to 3′ end of the sense strand (SS) via a phosphodiester linkage to form the following schematic:

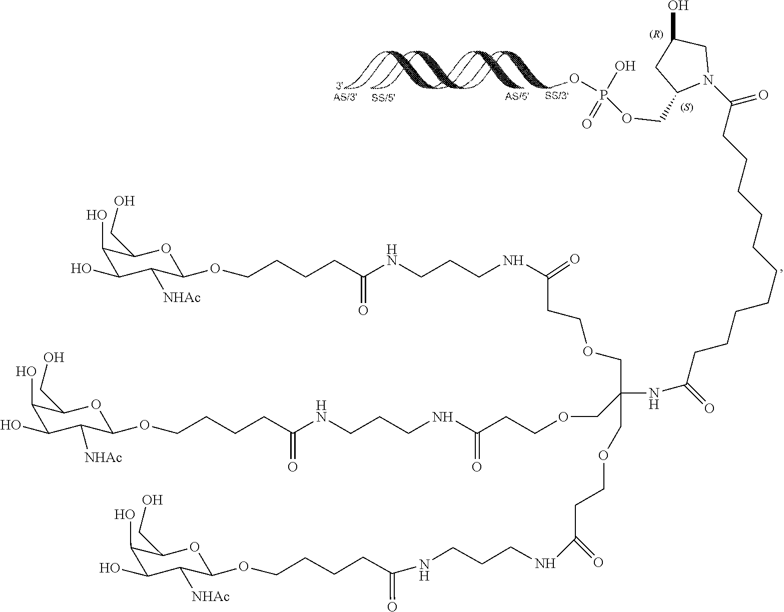

or a pharmaceutically acceptable salt thereof (e.g., sodium salt form).

In some embodiments, a double stranded RNAi agent includes:
(i) a sense strand (SS) comprising

(SEQ ID NO: 950)
5′-G005p001T005p001U004pU004pU004pG004pC007pU004pU007pU007pU007pG004pU004pA
004pA004pC004pU004pU004pG004p001A005p001A005pX1085-3′;

and
(ii) an antisense strand (AS) comprising

(SEQ ID NO: 1007)
5′-X033U1027p001U007p001C004pA004pA004pG007pU004pU004pA004pC004pA004pA004pA
004pA007pG004pC007pA004pA004pA004pA004pC004p001A004p001G004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage, and
X1085 is a ligand (L96),
   wherein the ligand (L96) is

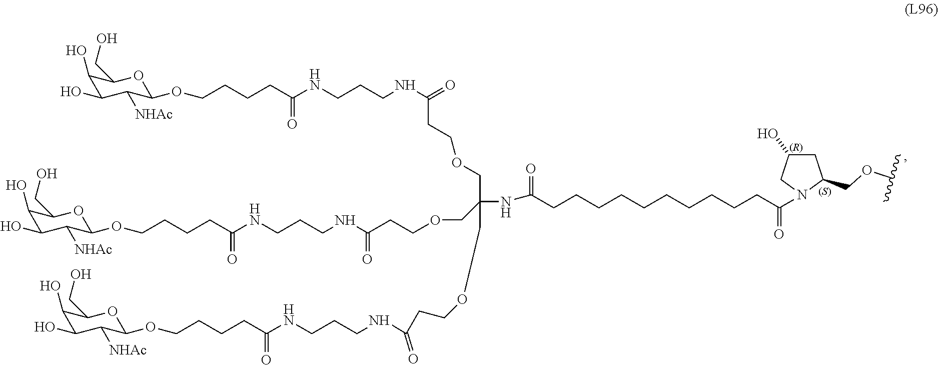

wherein the dsRNAi agent has the following schematic:

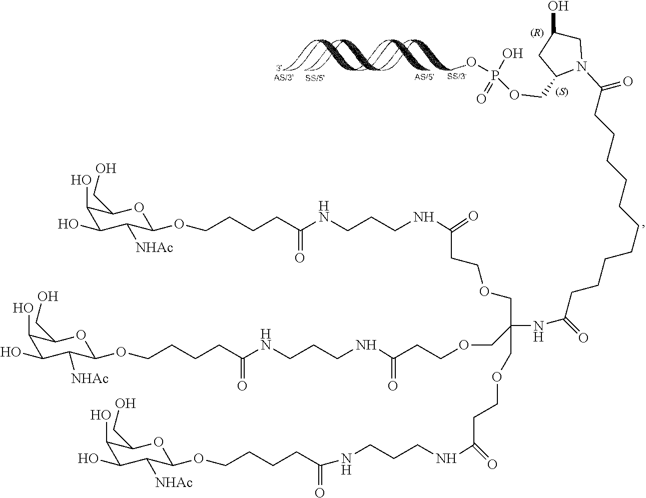

or a pharmaceutically acceptable salt thereof (e.g., sodium salt form).

In some embodiments, a double stranded RNAi agent includes:
(i) a sense strand (SS) consisting of

(SEQ ID NO: 950)
5′-G005p001T005p001U004pU004pU004pG004pC007pU004pU007pU007pU007pG004pU004pA
004pA004pC004pU004pU004pG004p001A005p001A005pX1085-3′;

and
(ii) an antisense strand (AS) consisting of

(SEQ ID NO: 1007)
5′-X033U1027p001U007p001C004pA004pA004pG007pU004pU004pA004pC004pA004pA004pA
004pA007pG004pC007pA004pA004pA004pA004pC004p001A004p001G004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage, and
X1085 is a ligand (L96),
   wherein the ligand (L96) is

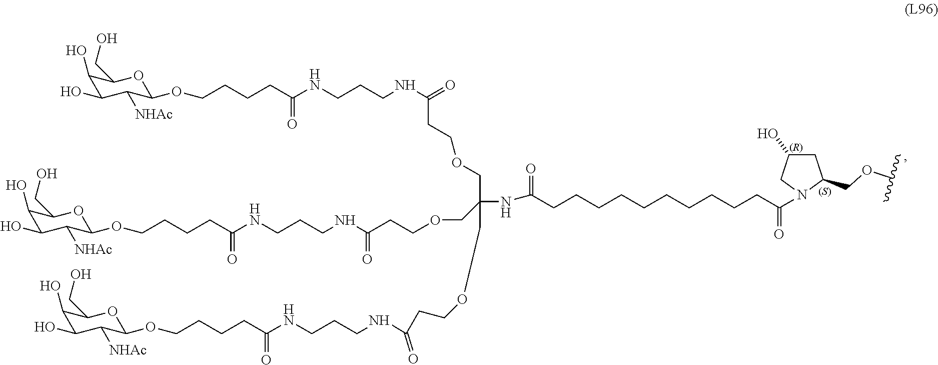

wherein the dsRNAi agent has the following schematic:

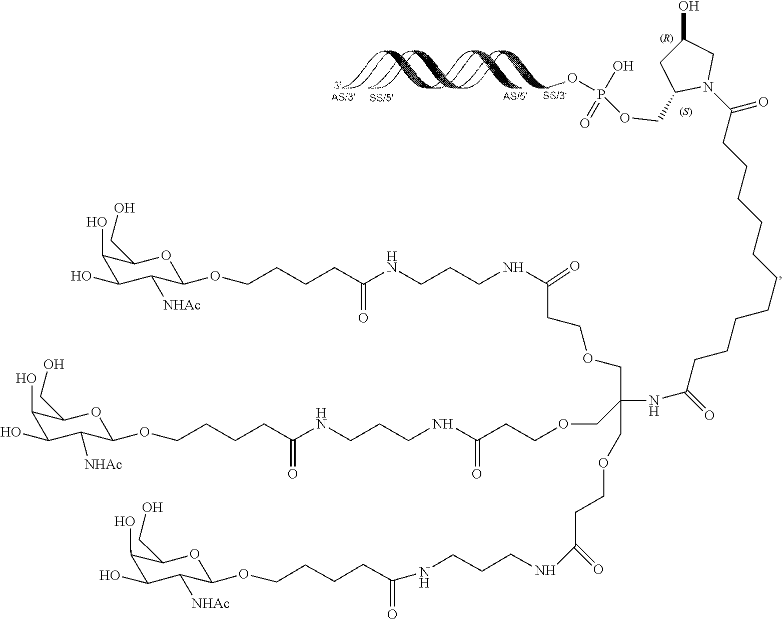

or a pharmaceutically acceptable salt thereof (e.g., sodium salt form).

In some embodiments, a double stranded RNAi agent (e.g., siRNA agent) includes:
(i) a sense strand (SS) comprising a nucleotide sequence of

(SEQ ID NO: 1016)
5′-C005p001T005p001A004pG004pA004pC004pC007pU004pG007pU007pT002pU004pU004pG
004pC004pU004pU004pU004pU004p001G005p001T005-3′;

(ii) an antisense strand (AS) comprising a nucleotide sequence of

(SEQ ID NO: 1022)
5′-X033A1027p001C007p001A042pA004pA004pA042pG004pC004pA004pA004pA004pA004pC
004pA007pG004pG007pU004pC004pU004pA004pG004p001A004p001A004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; X033A1027 is 5′-(E)-vinylphosphonate-2′-O-methyladenosine; p is a phosphodiester linkage; and p001 is a phosphorothioate linkage,
   wherein the ligand (L96) is

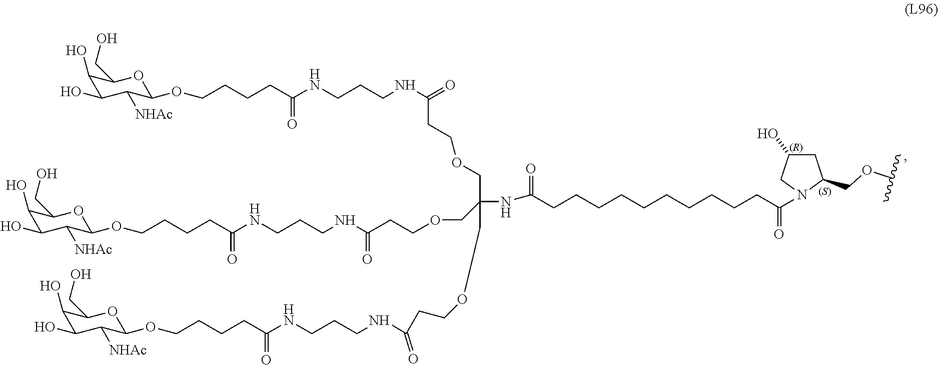

wherein the ligand is conjugated to 3′ end of the sense strand (SS) via a phosphodiester linkage to form the following schematic

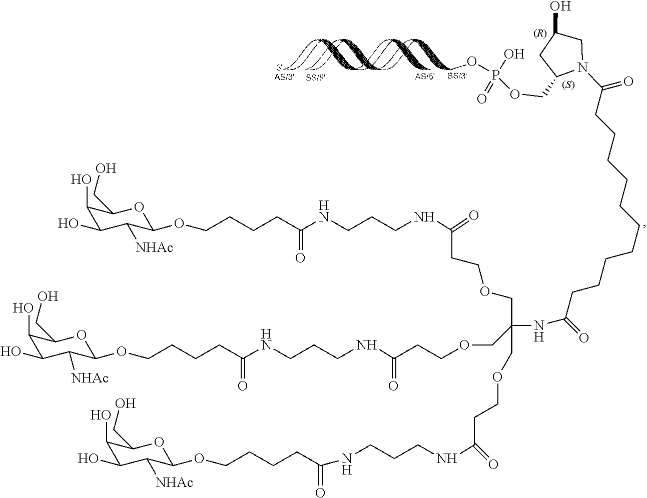

or a pharmaceutically acceptable salt thereof (e.g., sodium salt form).

In some embodiments, a double stranded RNAi agent (e.g., siRNA agent) includes:
(i) a sense strand (SS) consisting of a nucleotide sequence of:

(SEQ ID NO: 1016)
5′-C005p001T005p001A004pG004pA004pC004pC007pU004pG007pU007pT002pU004pU004pG
004pC004pU004pU004pU004pU004p001G005p001T005-3′;

(ii) an antisense strand (AS) consisting of a nucleotide sequence of:

(SEQ ID NO: 1022)
5′-X033A1027p001C007p001A042pA004pA004pA042pG004pC004pA004pA004pA004pA004pC
004pA007pG004pG007pU004pC004pU004pA004pG004p001A004p001A004-3′;

and
(iii) a ligand (L96),
   wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; X033A1027 is 5′-(E)-vinylphosphonate-2′-O-methyladenosine; p is a phosphodiester linkage; and p001 is a phosphorothioate linkage,
   wherein the ligand (L96) is

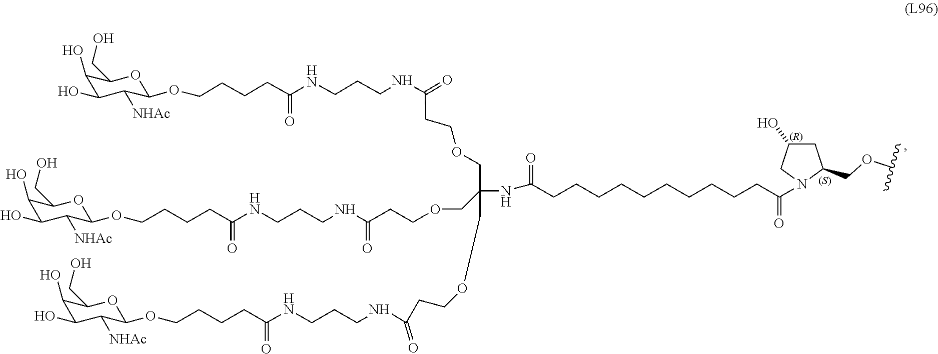

wherein the ligand is conjugated to 3′ end of the sense strand (SS) via a phosphodiester linkage to form the following schematic:

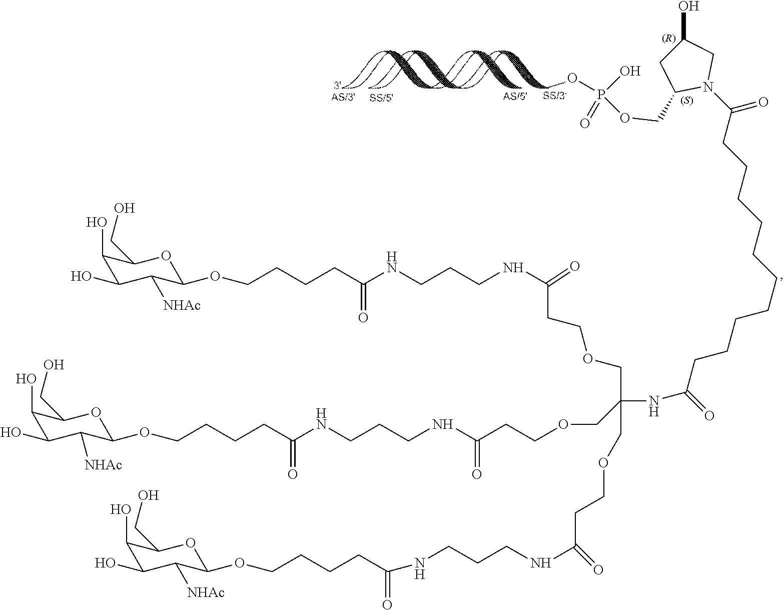

or a pharmaceutically acceptable salt thereof (e.g., sodium salt form).

In some embodiments, a double stranded RNAi agent includes:
(i) a sense strand (SS) comprising

(SEQ ID NO: 1028)
5′-C005p001T005p001A004pG004pA004pC004pC007pU004pG007pU007pT002pU004pU004pG
004pC004pU004pU004pU004pU004p001G005p001T005pX1085-3′;

and
(ii) an antisense strand (AS) comprising

(SEQ ID NO: 1034)
5′-X033A1027p001C007p001A042pA004pA004pA042pG004pC004pA004pA004pA004pA004pC
004pA007pG004pG007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; X033A1027 is 5′-(E)-vinylphosphonate-2′-O-methyladenosine; p is a phosphodiester linkage; and p001 is a phosphorothioate linkage, and
X1085 is a ligand (L96),
   wherein the ligand (L96) is

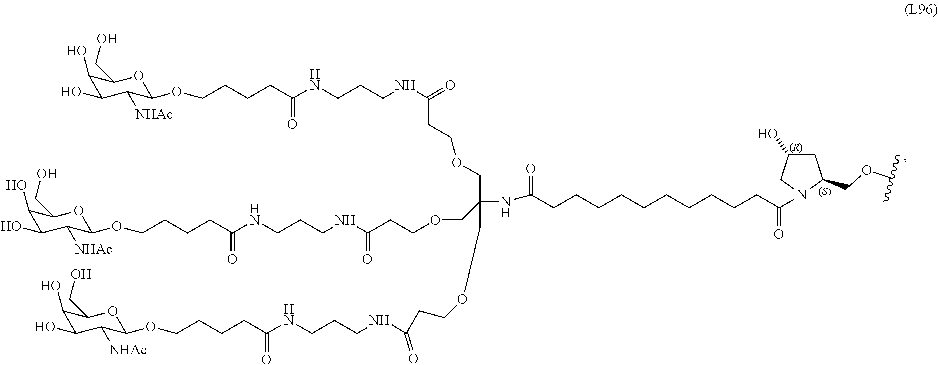

wherein the dsRNAi agent has the following schematic:

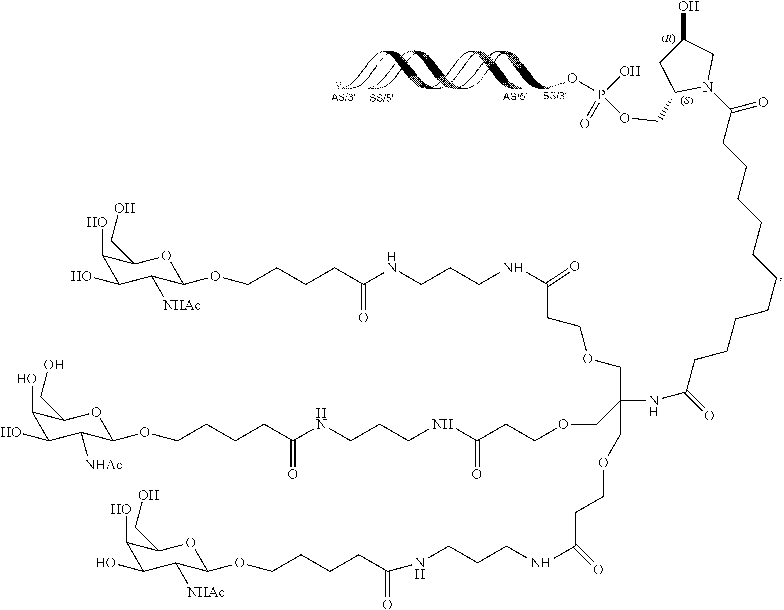

or a pharmaceutically acceptable salt thereof (e.g., sodium salt form).

In some embodiments, a double stranded RNAi agent includes:
(i) a sense strand (SS) consisting of

(SEQ ID NO: 1028)
5′-C005p001T005p001A004pG004pA004pC004pC007pU004pG007pU007pT002pU004pU004pG
004pC004pU004pU004pU004pU004p001G005p001T005pX1085-3′;

and
(ii) an antisense strand (AS) consisting of

(SEQ ID NO: 1034)
5′-X033A1027p001C007p001A042pA004pA004pA042pG004pC004pA004pA004pA004pA004pC
004pA007pG004pG007pU004pC004pU004pA004pG004p001A004p001A004-3′;

wherein:
T002 is 2′-deoxy thymidine (dT); A004 is 2′-O-methyladenosine; U004 is 2′-O-methyluridine; C004 is 2′-O-methylcytidine; G004 is 2′-O-methylguanosine; T005 is 2′-O-methoxyethyl(MOE)thymidine (or 5-methyl uridine); C005 is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is adenosine TNA; X033A1027 is 5′-(E)-vinylphosphonate-2′-O-methyladenosine; p is a phosphodiester linkage; and p001 is a phosphorothioate linkage, and
X1085 is a ligand (L96),
   wherein the ligand (L96) is

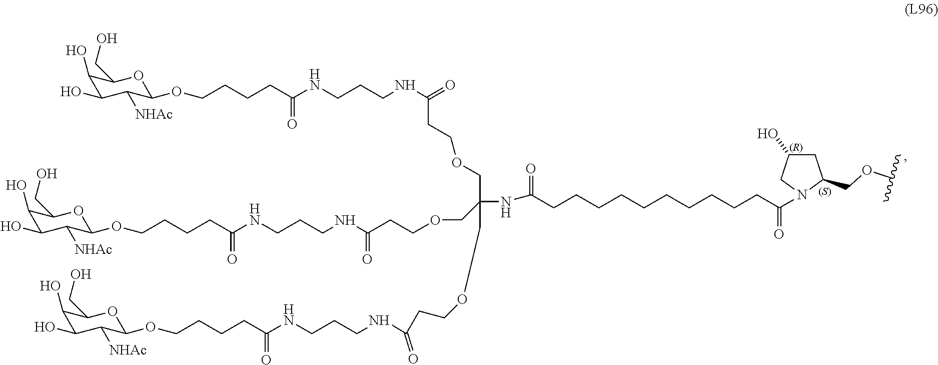

wherein the dsRNAi agent has the following schematic:

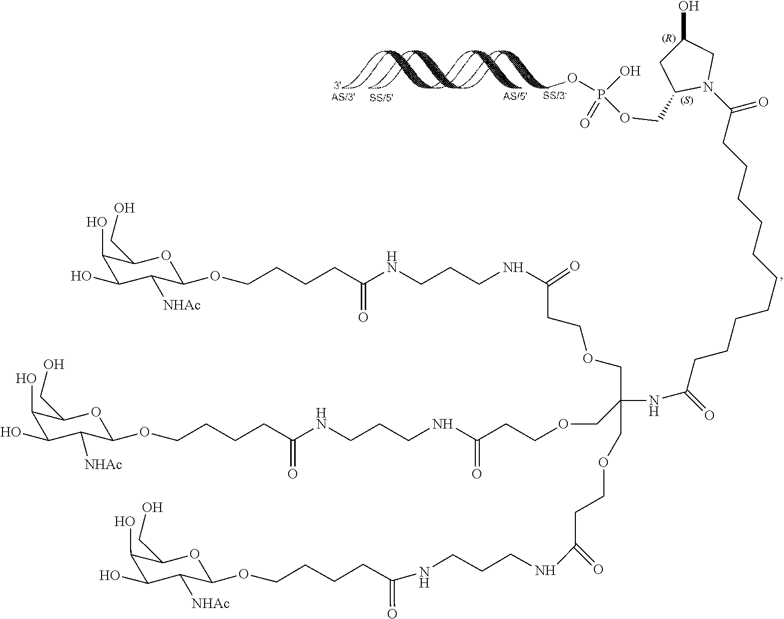

or a pharmaceutically acceptable salt thereof (e.g., sodium salt form).

### Pharmaceutical Compositions

Also provided herein is a pharmaceutical composition (“composition”) including the dsRNAi agents described herein (including all embodiments (e.g., Tables, Figures and Examples, and etc.) and a pharmaceutically acceptable carrier or excipient.

### Formulation

The pharmaceutical composition may be prepared and administered in a wide variety of dosage formulations. dsRNAi agents described herein may be administered orally, rectally, or by injection (e.g. intravenously, intramuscularly, intracutaneously, subcutaneously, intraduodenally, or intraperitoneally). In certain embodiments, the dsRNAi agents are formulated for injection (e.g. intravenously, intramuscularly, intracutaneously, subcutaneously, intraduodenally, or intraperitoneally). In certain embodiments, the dsRNAi agents are administered subcutaneously. In certain embodiments, the dsRNAi agents are administered intravenously.

For preparing pharmaceutical compositions from the dsRNAi agents described herein, the pharmaceutically acceptable carriers or excipients may be liquid. Particularly, when parenteral application (e.g., subcutaneously or intravenously administered) is needed or desired, particularly suitable admixtures for the compounds included in the pharmaceutical composition may be injectable, sterile solutions, oily or aqueous solutions, as well as suspensions, emulsions, or implants, including suppositories. In certain aspects, for parenteral injection (e.g., subcutaneous or intravenous administration), liquid form preparations may include solutions (e.g., a sterile aqueous solution), suspensions, emulsions (e.g., water solutions), aqueous or crystalline compositions, liposomal formulations, and micellar formulations. In some embodiments, the liquid form preparation is for an injectable device.

The pharmaceutically acceptable carriers or excipients can include buffers to adjust the pH to a desirable range for intravenous use. In some embodiments, the liquid form preparations for intravenous use may include a buffer solution containing acetate, citrate, prolamine, carbonate, phosphate, borate, sulfate, or any combination thereof, for example, the buffer solution is phosphate buffered saline (PBS). In some embodiments, the buffer solution may further include an agent to control the osmolarity, for example, proteins, peptides, amino acids, non-metabolized polymers, vitamins, ions, sugars, metabolites, organic acids, lipids, or salts (e.g., sodium chloride or potassium chloride).

In some embodiments, pharmaceutical compositions containing dsRNAi agent described herein include additional components to aid in delivery, stability, efficacy, or reduction of immunogenicity.

The pharmaceutical composition may include compositions wherein the active ingredient dsRNAi agent is contained in a therapeutically effective amount, i.e., in an amount effective to achieve its intended purpose (e.g., gene-silencing (e.g., inhibiting, downregulating, or suppressing of the gene) the expression of PCSK9 in a subject, or lowering LDL-C level in a subject). The actual amount effective for a particular application will depend, inter aim, on the condition being treated.

The pharmaceutical compositions described herein typically include a therapeutically effective amount of dsRNAi agents described herein. In some embodiments, a therapeutically effective amount of the dsRNAi agent targeting the PCSK9 (e.g., human PCSK9) can reduce PCSK9 mRNA levels in a treated cell or subject by at least about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to non-treated or control cell or subject. In some embodiments, a therapeutically effective amount of an RNAi agent targeting PCSK9 can reduce PCSK9 protein levels in a treated cell or subject by at least about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to non-treated or control cell or subject. In some embodiments, a therapeutically effective amount of an RNAi agent targeting PCSK9 can reduce PCSK9 protein levels in a treated cell or subject by at least about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to non-treated or control cell or subject.

A given clinical treatment (e.g., lowering LCL-C level in blood or serum of a subject) is considered effective where there is at least about 10%, about 15%, about 20%, about 25%, about 30%, about 35%, about 40%, about 45%, about 50%, about 55%, about 60%, about 65%, about 70%, about 75%, about 80%, about 85%, about 90% or about 95% reduction in a measurable parameter associated with a disease or disorder. In some embodiments, the given clinical treatment (e.g., lowering LCL-C level in blood or serum of a subject) is considered effective where there is at least about 10%, about 15%, about 20%, about 25%, about 30%, about 35%, about 40%, about 45%, about 50%, about 55%, about 60%, about 65%, about 70%, about 75%, about 80%, about 85%, about 90% or about 95% reduction in a measurable parameter associated with a disease or disorder. In some embodiments, therapeutically effective amount of a dsRNAi agent for the treatment of that disease or disorder (e.g., lowering LCL-C level in blood or serum of a subject) is the amount necessary to effect at least about 10%, about 15%, about 20%, about 25%, about 30%, about 35%, about 40%, about 45%, about 50%, about 55%, about 60%, about 65%, about 70%, about 75%, about 80%, about 85%, about 90% or about 95% at least about 10%, about 15%, about 20%, about 25%, about 30%, about 35%, about 40%, about 45%, about 50%, about 55%, about 60%, about 65%, about 70%, about 75%, about 80%, about 85%, about 90% or about 95% reduction, respectively, in that parameter. In some embodiments, therapeutically effective amount of a dsRNAi agent for the treatment of that disease or disorder (e.g., lowering LCL-C level in blood or serum of a subject) is the amount necessary to effect at least about 40%, about 45%, about 50%, about 55%, about 60%, about 65%, about 70%, about 75%, about 80%, about 85%, or about 90%, respectively, in that parameter.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Dosage amounts and intervals can be adjusted individually to provide levels of the administered dsRNAi agents that is effective for the particular clinical indication being treated. This will provide a therapeutic regimen that is commensurate with the severity of the individual's disease state.

The effective prophylactic or therapeutic treatment regimen as described herein can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

### Toxicity

The ratio between toxicity and therapeutic effect for the dsRNAi agent is its therapeutic index and can be expressed as the ratio between LD₅₀ (the amount of compound lethal in 50% of the population) and ED₅₀ (the amount of compound effective in 50% of the population). The dsRNAi agents that exhibit high therapeutic indices are preferred. Therapeutic index data obtained from cell culture assays and/or animal studies can be used in formulating a range of dosages for use in humans. The dosage of such dsRNAi agents preferably lies within a range of plasma concentrations that include the ED₅₀ with little or no toxicity. The dosage may vary within this range depending upon the dosage form employed and the route of administration utilized. The exact formulation, route of administration, and dosage may also be chosen by the individual physician in view of the patient's condition and the particular method in which the dsRNAi agents are used.

### Methods of Use

The methods and compositions of the present disclosure, e.g., the methods and PCSK9 RNAi agent compositions, can be used in any appropriate dosage and/or composition described herein or known in the art, as well as with any suitable route of administration described herein or known in the art. Any aspects or embodiments disclosed herein that are not mutually exclusive can be combined.

In an aspect, the disclosure provides a method of or a use for inhibiting expression of PCSK9 (e.g., gene or protein) in a subject. The method or the use includes administering to the subject the dsRNAi agent or a pharmaceutical composition as described herein. In some embodiments, the method includes administering to the subject a therapeutically effective amount of the dsRNAi agent or a pharmaceutical composition as described herein.

In an aspect, the disclosure provides a method of, or a use for inhibiting expression of PCSK9 (e.g., gene or protein) in a subject. The method or the use includes administering to the subject the dsRNAi agent including the siRNA selected from Tables 1-2 and 5-6, or a pharmaceutical composition thereof. In some embodiments, the method includes administering to the subject a therapeutically effective amount of the dsRNAi agent including the siRNA selected from Tables 1-2 and 5-6, or a pharmaceutical composition thereof.

In certain aspects, the disclosure provides a method of, or for a use for treating or preventing the PCSK9-associated disorder or disease by reduction in PCSK9 expression. The method or the use includes administering to the subject the dsRNAi agent including the siRNA selected from Tables 1-2 and 5-6, or a pharmaceutical composition thereof. In some embodiments, the method includes administering to the subject a therapeutically effective amount of the dsRNAi agent including the siRNA selected from Tables 1-2 and 5-6, or a pharmaceutical composition thereof. The level of PCSK9 may be measured or detected in a sample (e.g., a blood, serum, or liver tissue) from the subject. In certain aspects, the method may include treating or preventing one or more symptoms in the subject having the PCSK9-associated disorder or disease. In some embodiments, the methods may decrease PCSK9 protein accumulation.

Exemplary PCSK9-associated disorders or diseases include acquired or inherited disorders of lipid metabolism in a subject in need thereof. In some embodiments, PCSK9-associated disorders or diseases include any disorder associated with or caused by a disturbance in lipid metabolism, e.g., abnormal elevation of levels of any or all lipids and/or lipoproteins in the blood or a condition that can lead to abnormal elevation of levels of any or all lipids and/or lipoproteins in the blood, such as a hyperlipidemia, and other forms of lipid imbalance such as hypercholesterolemia, hypertriglyceridemia, mixed hyperlipidemia, as well as the pathological conditions associated with these disorders, e.g., congestive heart disease (CHD) and atherosclerosis.

In some embodiments, the method is for treating a lipid disorder. In some embodiments, the method is for treating hyperlipidemia. In some embodiments, the method is for treating hypercholesterolemia. In some embodiments, the method is for treating hypertriglyceridemia. In some embodiments, the method is for treating mixed hyperlipidemia. In some embodiments, the method is for treating congestive heart disease (CHD). In some embodiments, the method is for treating atherosclerosis. In some embodiments, the method is for treating dyslipidemia.

In some embodiments, the method is for preventing a lipid disorder. In some embodiments, the method is for preventing hyperlipidemia. In some embodiments, the method is for preventing hypercholesterolemia. In some embodiments, the method is for preventing hypertriglyceridemia. In some embodiments, the method is for preventing mixed hyperlipidemia. In some embodiments, the method is for preventing congestive heart disease (CHD). In some embodiments, the method is for preventing atherosclerosis. In some embodiments, the method is for preventing dyslipidemia.

In an aspect, the disclosure also provides a method of, or a use for lowering a level of low-density lipoprotein cholesterol (LDL-C) in a subject in need thereof. The method or the use includes administering to the subject the dsRNAi agent including the siRNA selected from Tables 1-2 and 5-6, or a pharmaceutical composition thereof. In some embodiments, the method includes administering to the subject a therapeutically effective amount of the dsRNAi agent including the siRNA selected from Tables 1-2 and 5-6, or a pharmaceutical composition thereof. In some embodiments, the level of low-density lipoprotein cholesterol (LDL-C) can be measured in a blood vessel of a subject in need thereof. The method includes administering to the subject the dsRNAi agent or a pharmaceutical composition as described herein. In some embodiments, the method includes administering to the subject a therapeutically effective amount of the dsRNAi agent or a pharmaceutical composition as described herein.

In an aspect, the disclosure provides a method of, or a use for treating or preventing hyperlipidemia in a subject in need thereof. The method or the use includes administering to the subject the dsRNAi agent including the siRNA selected from Tables 1-2 and 5-6, or a pharmaceutical composition thereof. In some embodiments, the method includes administering to the subject a therapeutically effective amount of the dsRNAi agent including the siRNA selected from Tables 1-2 and 5-6, or a pharmaceutical composition thereof. The method includes administering to the subject the dsRNAi agent or a pharmaceutical composition as described herein. In some embodiments, the method includes administering to the subject a therapeutically effective amount of the dsRNAi agent or a pharmaceutical composition as described herein.

In an aspect, the disclosure provides a method of, or a use for treating or preventing atherosclerotic cardiovascular disease (ASCVD) in a subject in need thereof. The method includes administering to the subject the dsRNAi agent or a pharmaceutical composition as described herein. In some embodiments, the method or the use includes administering to the subject the dsRNAi agent including the siRNA selected from Tables 1-2 and 5-6, or a pharmaceutical composition thereof. In some embodiments, the method includes administering to the subject a therapeutically effective amount of the dsRNAi agent including the siRNA selected from Tables 1-2 and 5-6, or a pharmaceutical composition thereof.

In an aspect, the disclosure provides a method of, or a use for reducing or preventing cardiovascular event in a subject in need thereof. The method includes administering to the subject the dsRNAi agent or a pharmaceutical composition as described herein. In some embodiments, the method or the use includes administering to the subject the dsRNAi agent including the siRNA selected from Tables 1-2 and 5-6, or a pharmaceutical composition thereof. In some embodiments, the method includes administering to the subject a therapeutically effective amount of the dsRNAi agent including the siRNA selected from Tables 1-2 and 5-6, or a pharmaceutical composition thereof. In some embodiments, the cardiovascular event is cardiovascular death, non-fatal myocardial infarction (MI), non-fatal ischemic stroke, urgent coronary revascularization, coronary heart disease (CHD) death, or any combination thereof.

In an aspect, the disclosure provides a method of, or a use for reducing or preventing a major limb adverse event (MALE) in a subject in need thereof. The method includes administering to the subject the dsRNAi agent or a pharmaceutical composition as described herein. In some embodiments, the method or the use includes administering to the subject the dsRNAi agent including the siRNA selected from Tables 1-2 and 5-6, or a pharmaceutical composition thereof. In some embodiments, the method includes administering to the subject a therapeutically effective amount of the dsRNAi agent including the siRNA selected from Tables 1-2 and 5-6, or a pharmaceutical composition thereof.

In some embodiments, the method or the use includes administering to the subject the dsRNAi agent including the siRNA selected from Tables 1-2 and 5-6, or a pharmaceutical composition thereof. In some embodiments, the method includes administering to the subject a therapeutically effective amount of the dsRNAi agent including the siRNA selected from Tables 1-2 and 5-6, or a pharmaceutical composition thereof.

In some embodiments, the MALE is acute lower limb ischemia, lower limb amputation due to ischemia, urgent lower limb revascularization for ischemia, or any combination thereof.

In certain aspects, for the methods described above, administration of the dsRNAi agent or the pharmaceutical composition may be, but not limited to, subcutaneous, intravenous, intramuscular, intraocular, intrabronchial, intrapleural, intraperitoneal, intraarterial, lymphatic, cerebrospinal, and any combinations thereof. In some embodiments, the dsRNAi agent or the pharmaceutical composition may be administered subcutaneously or intravenously.

In some embodiments, the dsRNAi agent is be delivered locally (e.g., to the site of the disease, such as a liver) so that levels of PCSK9 outside the diseased areas can be maintained as close to normal as possible. In some embodiments, the level of PCSK9 in the body can be modulated such that it is low enough to improve or ameliorate the disease state, but not so low that organ pathology occurs.

In some embodiments, the administration is via a depot injection that can release the dsRNAi agent in a consistent way over a prolonged time period, so as to reduce the frequency of dosing needed to obtain a desired effect, e.g., a desired inhibition of PCSK9, or a therapeutic or prophylactic effect and/or to provide more consistent serum concentrations of the therapeutic agent. In some embodiments, the administration is via a pump, e.g., external pump or a surgically implanted pump. For example, the pump is a subcutaneously implanted osmotic pump or an infusion pump for intravenous, subcutaneous, arterial, or epidural infusions. In some embodiments, the pump is a surgically implanted pump that delivers the dsRNAi agent described herein directly or closely to the liver.

In some embodiments, the subject is a human. In some embodiments, the subject has or diagnosed with hypercholesterolemia. In some embodiments, the subject has or diagnosed with PCSK9-associated disorder or disease.

In some embodiments, the subject after treatment (e.g., after administering the dsRNAi agent or the pharmaceutical composition described herein) does not have a muscle side effect. In some embodiments, the subject the after treatment (e.g., after administering the dsRNAi agent or the pharmaceutical composition described herein) does not have a skeletal muscle side effect (e.g., muscle AE).

In an aspect, the disclosure provides a method of, or a use for inhibiting expression of PCSK9 (e.g., gene or protein) in a subject in need thereof. In some embodiments, the method or the use includes administering to the subject the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883; or (g) a sense strand comprising SEQ ID NO: 1016, and an antisense strand comprising SEQ ID NO: 1022, or a pharmaceutical composition thereof. In some embodiments, the method includes administering to the subject a therapeutically effective amount of the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883, or (g) a sense strand comprising SEQ ID NO: 1016, and an antisense strand comprising SEQ ID NO: 1022; or a pharmaceutical composition thereof. In some embodiments, the dsRNAi agent includes a ligand. In some embodiments, the ligand may directly (e.g., covalently) conjugated to at least one strand of the dsRNA. In some embodiments, the ligand includes one or more GalNAc moieties. In some embodiments, the ligand includes a GalNAc ligand described herein.

In an aspect, the disclosure provides a method of, or for a use for treating or preventing the PCSK9-associated disorder or disease by reduction in PCSK9 expression in a subject in need thereof. The method or the use includes administering to the subject the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883, or (g) a sense strand comprising SEQ ID NO: 1016, and an antisense strand comprising SEQ ID NO: 1022; or a pharmaceutical composition thereof. The level of PCSK9 may be measured or detected in a sample (e.g., a blood, serum, or liver tissue) from the subject. In certain aspects, the method may include treating or preventing one or more symptoms in the subject having the PCSK9-associated disorder or disease. In some embodiments, the methods may decrease PCSK9 protein accumulation. In some embodiments, the dsRNAi agent includes a ligand. In some embodiments, the ligand may directly (e.g., covalently) conjugated to at least one strand of the dsRNA. In some embodiments, the ligand includes one or more GalNAc moieties. In some embodiments, the ligand includes a GalNAc ligand described herein.

In an aspect, the disclosure also provides a method of, or a use for lowering a level of low-density lipoprotein cholesterol (LDL-C) in a subject in need thereof. The method or the use includes administering to the subject the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883, or (g) a sense strand comprising SEQ ID NO: 1016, and an antisense strand comprising SEQ ID NO: 1022; or a pharmaceutical composition thereof. In some embodiments, the method includes administering to the subject a therapeutically effective amount of the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883, or (g) a sense strand comprising SEQ ID NO: 1016, and an antisense strand comprising SEQ ID NO: 1022; or a pharmaceutical composition thereof. In some embodiments, the level of low-density lipoprotein cholesterol (LDL-C) can be measured in a blood vessel of a subject in need thereof. The method includes administering to the subject the dsRNAi agent or a pharmaceutical composition as described herein. In some embodiments, the method includes administering to the subject a therapeutically effective amount of the dsRNAi agent or a pharmaceutical composition as described herein. In some embodiments, the dsRNAi agent includes a ligand. In some embodiments, the ligand may directly (e.g., covalently) conjugated to at least one strand of the dsRNA. In some embodiments, the ligand includes one or more GalNAc moieties. In some embodiments, the ligand includes a GalNAc ligand described herein.

In an aspect, the disclosure provides a method of, or a use for treating or preventing atherosclerotic cardiovascular disease (ASCVD) in a subject in need thereof. In some embodiments, the method or the use includes administering to the subject the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; or (f) a sense strand comprising SEQ ID NO: 830, and an antisense strand comprising SEQ ID NO: 883, or a pharmaceutical composition thereof. In some embodiments, the method includes administering to the subject a therapeutically effective amount of the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883, or (g) a sense strand comprising SEQ ID NO: 1016, and an antisense strand comprising SEQ ID NO: 1022; or a pharmaceutical composition thereof. In some embodiments, the dsRNAi agent includes a ligand. In some embodiments, the ligand may directly (e.g., covalently) conjugated to at least one strand of the dsRNA. In some embodiments, the ligand includes one or more GalNAc moieties. In some embodiments, the ligand includes a GalNAc ligand described herein.

In an aspect, the disclosure provides a method of, or a use for reducing or preventing cardiovascular event in a subject in need thereof. In some embodiments, the method or the use includes administering to the subject the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883, or (g) a sense strand comprising SEQ ID NO: 1016, and an antisense strand comprising SEQ ID NO: 1022; or a pharmaceutical composition thereof. In some embodiments, the method includes administering to the subject a therapeutically effective amount of the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883, or (g) a sense strand comprising SEQ ID NO: 1016, and an antisense strand comprising SEQ ID NO: 1022; or a pharmaceutical composition thereof. In some embodiments, the cardiovascular event is cardiovascular death, non-fatal myocardial infarction (MI), non-fatal ischemic stroke, urgent coronary revascularization, coronary heart disease (CHD) death, or any combination thereof. In some embodiments, the dsRNAi agent includes a ligand. In some embodiments, the ligand may directly (e.g., covalently) conjugated to at least one strand of the dsRNA. In some embodiments, the ligand includes one or more GalNAc moieties. In some embodiments, the ligand includes a GalNAc ligand described herein.

In an aspect, the disclosure provides a method of, or a use for reducing or preventing a major limb adverse event (MALE) in a subject in need thereof. In some embodiments, the method or the use includes administering to the subject the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883, or (g) a sense strand comprising SEQ ID NO: 1016, and an antisense strand comprising SEQ ID NO: 1022; or a pharmaceutical composition thereof. In some embodiments, the method includes administering to the subject a therapeutically effective amount of the dsRNAi agent including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883, or (g) a sense strand comprising SEQ ID NO: 1016, and an antisense strand comprising SEQ ID NO: 1022; or a pharmaceutical composition thereof. In some embodiments, the dsRNAi agent includes a ligand. In some embodiments, the ligand may directly (e.g., covalently) conjugated to at least one strand of the dsRNA. In some embodiments, the ligand includes one or more GalNAc moieties. In some embodiments, the ligand includes a GalNAc ligand described herein.

In some embodiments, the dsRNAi including (a) a sense strand comprising SEQ ID NO: 800, and an antisense strand comprising SEQ ID NO: 853; (b) a sense strand comprising SEQ ID NO: 801, and an antisense strand comprising SEQ ID NO: 854; (c) a sense strand comprising SEQ ID NO: 806, and an antisense strand comprising SEQ ID NO: 859; (d) a sense strand comprising SEQ ID NO: 811, and an antisense strand comprising SEQ ID NO: 864; (e) a sense strand comprising SEQ ID NO: 813, and an antisense strand comprising SEQ ID NO: 866; (f) a sense strand comprising SEQ ID NO: 830, and an antisense strand comprising SEQ ID NO: 883 or (g) a sense strand comprising SEQ ID NO: 1016, and an antisense strand comprising SEQ ID NO: 1022 may be conjugated to one or more ligands (e.g., L96 or X2000) as described herein.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

### Kit

In an aspect, provided is a kit including the dsRNAi agent or the pharmaceutical composition as described herein. In certain aspects, the kit further includes one or more applicator.

In certain aspects, the kit may include a suitable container containing a pharmaceutical composition (e.g., PCSK9 dsRNAi agent) as described herein. In some embodiments, the container may be a vial or a pre-filled syringe. In certain aspects, the kit may include a suitable applicator (e.g., an injection device) for parenterally (e.g., subcutaneously or intravenously) administering the pharmaceutical composition (e.g., PCSK9 dsRNAi agent) as described herein. In some embodiments, the kit includes a syringe as an applicator and optionally include a needle (e.g., with or without cannula). In some embodiments, the kit includes a pre-filled syringe containing the pharmaceutical composition (e.g., PCSK9 dsRNAi agent), optionally with a needle (e.g., with or without cannula).

### EMBODIMENTS

Embodiment 1: A double stranded RNAi (dsRNAi) agent comprising:
(i) a sense strand comprising a nucleotide sequence selected from SEQ ID Nos. 3 to 381; and
(ii) an antisense strand forming a duplex with the sense strand and comprising a nucleotide sequence selected from SEQ ID Nos. 382 to 760.

Embodiment 2: A double stranded RNAi (dsRNAi) agent comprising:
(i) a sense strand comprising a nucleotide sequence selected from SEQ ID Nos. 761 to 776; and
(ii) an antisense strand forming a duplex with the sense strand and comprising a nucleotide sequence selected from SEQ ID Nos. 777 to 792.

Embodiment 3: The dsRNAi agent of Embodiment 1 or 2, wherein one or more nucleotides in the sense strand and the antisense strand are modified nucleotides

Embodiment 4: The dsRNAi agent of any one of Embodiments 1 to 3, wherein each of the modified nucleotides independently comprises one or more modifications selected from a 2′-deoxy modification, a 2′-O-alkyl modification, a 2′-halo modification, a threofuranosyl nucleotide (TNA) modification, a 2′-5′-linkage modification, a conformationally restricting modification, an abasic modification, a 2′-amino-modification, a 2′-O-allyl modification, 2′-C-alkyl modification, a 2′-O-alkoxyalkyl modification, a morpholino modification, a phosphoramidate modification, a non-natural nucleobase modification, a modification in a tetrahydropyran, a modification containing a 1,5-anhydrohexitol, a modification containing a cyclohexenyl, a modification containing a phosphorothioate group, a modification containing a 5′-vinyl-phosphonate, a modification containing a 5′-phosphate, a modification to form a thermally destabilizing nucleotide, a glycol nucleic acid (GNA) modification, and a 2-O—(N-methylacetamide) modification.

Embodiment 5: The dsRNAi agent of Embodiment 4, wherein each of the modified nucleotides independently comprises one or more modifications selected from 2′-deoxy modification, 2′-O-alkoxyalkyl modification, 2′-O-alkyl modification, 2′-O-allyl modification, 2′-C-allyl modification, 2′-halo modification, modification containing a non-natural nucleobase, GNA modification, and TNA modification.

Embodiment 6: The dsRNAi agent of any one of Embodiments 1 to 5, wherein the dsRNAi agent comprises a 3′-phosphorothioate (PS) modification.

Embodiment 7: The dsRNAi agent of any one of Embodiments 3 to 6, wherein each of the modified nucleotides independently comprises one or more modifications selected from 2′-deoxy modification, 2′-O-methyl (2′-OMe) modification, 2′-fluoro (2′-F) modification, 2′-O-methoxyethyl (2′-MOE) modification, the modification containing a non-natural nucleobase, TNA, GNA, 3′-phosphorothioate (PS) modification, and 5′-vinyl-phosphonate (5′-VP) modification.

Embodiment 8: The dsRNAi agent of any one of Embodiments 1 to 7, wherein the sense strand comprises one or two 2′-MOE modifications positioned at the 1st and/or 2nd nucleotides from the 5′ end.

Embodiment 9: The dsRNAi agent of any one of Embodiments 1 to 8, wherein the sense strand comprises one or two 2′-MOE modifications positioned at the 1st and/or 2nd nucleotides from the 3′ end.

Embodiment 10: The dsRNAi agent of any one of Embodiments 1 to 7, wherein the sense strand comprises one or two TNAs positioned at the 1st and/or 2nd nucleotides from the 5′ end.

Embodiment 11: The dsRNAi agent of any one of Embodiments 1 to 7 and 10, wherein the sense strand comprises one or two TNAs positioned at the 1st and/or 2nd nucleotides from the 3′ end.

Embodiment 12: The dsRNAi agent of any one of Embodiments 1 to 11, wherein the antisense strand comprises a 5′-VP modification at the 1st nucleotide from the 5′ end.

Embodiment 13: The dsRNAi agent of any one of Embodiments 1 to 12, wherein the antisense strand comprises a 5′-VP-2′-OMe modification at the 1st position from the 5′ end.

Embodiment 14: The dsRNAi agent of any one of Embodiments 1 to 13, wherein each of the sense strand and the antisense strand independently comprises two, three, four, five or six 2′-F modified nucleotides.

Embodiment 15: The dsRNAi agent of any one of Embodiments 1 to 14, wherein the sense strand comprises one or two 3′-PS modifications at the 1st and/or 2nd nucleotides from the 5′ end.

Embodiment 16: The dsRNAi agent of any one of Embodiments 1 to 15, wherein the sense strand comprises one or two 3′-PS modifications at the 1st and/or 2nd nucleotides from the 3′ end.

Embodiment 17: The dsRNAi agent of any one of Embodiments 1 to 16, wherein the antisense strand comprises one or two 3′-PS modifications at the 1st and/or 2nd nucleotides from the 5′ end, and/or one or two 3′-PS modifications at the 1st and/or 2nd nucleotides from the 3′ end.

Embodiment 18: The dsRNAi agent of any one of Embodiments 1 to 17, wherein the sense strand is 21 nucleotides in length and the antisense strand is 23 nucleotides in length.

Embodiment 19: The dsRNAi agent of Embodiment 18, wherein the sense strand comprises one to four 2′-MOE modifications positioned at the 1st, 2nd, 20th, and/or 21st nucleotides from the 5′ end.

Embodiment 20: The dsRNAi agent of any one of Embodiments 18 to 19, wherein the sense strand does not comprise a 2′-MOE modification at the 3rd to 19th positions from the 5′ end.

Embodiment 21: The dsRNAi agent of Embodiment 18, wherein the sense strand comprises one to four TNAs positioned at the 1st, 2nd, 20th, and/or 21st nucleotides from the 5′ end.

Embodiment 22: The dsRNAi agent of any one of Embodiments 18 and 21, wherein the sense strand does not comprise a 2′-MOE modifications and TNA at the 3rd to 19th positions from the 5′ end.

Embodiment 23: The dsRNAi agent of any one of Embodiments 18 to 22, wherein the sense strand comprises two, three, or four 2′-F modifications positioned at the 7th, 9th, 10th, and/or 11th nucleotides from the 5′ end.

Embodiment 24: The dsRNAi agent of any one of Embodiments 18 to 23, wherein the sense strand comprises one or two 2′-deoxy modifications positioned at the 10th and/or 11th nucleotides from the 5′ end.

Embodiment 25: The dsRNAi agent of any one of Embodiments 18 to 24, wherein the sense strand comprises (i) 2′-F modifications positioned at the 7th, 9th, and 10th nucleotides from the 5′ end and (ii) a 2′-deoxy modification positioned at the 11th nucleotide from the 5′ end.

Embodiment 26: The dsRNAi agent of any one of Embodiments 19 to 25, wherein the remaining nucleotides in the sense strand comprise 2′-OMe modifications.

Embodiment 27: The dsRNAi agent of any one of Embodiments 18 to 26, wherein the antisense strand comprises a 5′-(E)-VP modification at the 1st nucleotide from the 5′ end.

Embodiment 28: The dsRNAi agent of any one of Embodiments 18 to 26, wherein the antisense strand comprises a 5′-(E)-VP-2′-OMe modification at the 1st nucleotide from the 5′ end.

Embodiment 29: The dsRNAi agent of any one of Embodiments 18 to 28, wherein the antisense strand comprises two, three, or four 2′-F modifications positioned at the 2nd, 6th, 14th, and/or 16th nucleotides from the 5′ end.

Embodiment 30: The dsRNAi agent of any one of Embodiments 18 to 29, wherein the antisense strand comprises 2′-F modifications positioned at the 2nd, 6th, 14th, and 16th nucleotides from the 5′ end.

Embodiment 31: The dsRNAi agent of any one of Embodiments 18 to 29, wherein the antisense strand comprises 2′-F modifications positioned at the 2nd, 6th, 14th, and 16th nucleotides from the 5′ end and a TNA positioned at the 3rd nucleotide from the 5′ end.

Embodiment 32: The dsRNAi agent of any one of Embodiments 18 to 29, wherein the antisense strand comprises 2′-F modifications positioned at the 2nd, 6th, 14th, and 16th nucleotides from the 5′ end and a TNA, GNA or 2′-deoxy modification positioned at the 5th nucleotide from the 5′ end.

Embodiment 33: The dsRNAi agent of any one of Embodiments 18 to 29, wherein the antisense strand comprises 2′-F modifications positioned at the 2nd, 14th, and 16th nucleotides from the 5′ end and a TNA, GNA or 2′-deoxy modification positioned at the 6th nucleotide from the 5′ end.

Embodiment 34: The dsRNAi agent of any one of Embodiments 18 to 29, wherein the antisense strand comprises 2′-F modifications positioned at the 2nd, 6th, 14th, and 16th nucleotides and a TNA, GNA or 2′-deoxy modification positioned at the 7th nucleotide.

Embodiment 35: The dsRNAi agent of any one of Embodiments 27 to 34, wherein the remaining nucleotides in antisense strand comprise 2′-OMe modified modifications.

Embodiment 36: The dsRNAi agent of any one of Embodiments 18 to 35, wherein the sense strand comprises one to eight 3′-PS group at the 1st, 2nd, 3rd, 4th, 17th, 18th, 19th and/or 20th nucleotides from the 5′ end.

Embodiment 37: The dsRNAi agent of any one of Embodiments 18 to 36, wherein the antisense strand comprises one to eight 3′-PS group at the 1st, 2nd, 3rd, 4th, 19th, 20th, 21st and/or 22nd nucleotides from the 5′ end.

Embodiment 38: The dsRNAi agent of any one of Embodiments 15, 16, 17, 36 and 37, wherein at least one of the 3′-PS groups in each sense strand and antisense strand has a stereopure Rp configuration.

Embodiment 39: The dsRNAi agent of any one of Embodiments 15, 16, 17, 36 and 37, wherein at least one of the 3′-PS groups in each sense strand and antisense strand has a stereopure Sp configuration.

Embodiment 40: A double stranded RNAi (dsRNAi) agent comprising:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883

Embodiment 41: The dsRNAi agent of any one of Embodiments 1 through 40, further comprising a ligand.

Embodiment 42: The dsRNAi agent of Embodiment 1, wherein the ligand comprises a N-acetylgalactosamine (GalNAc) moiety.

Embodiment 43: The dsRNAi agent of Embodiment 1 or 42, wherein the ligand has a structure of:

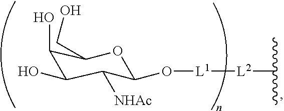

wherein:
each L¹ is independently a linker which may be same or different in each occurrence;
L² is a linker;
n is an integer from 1 to 3; and
[symbol] is an attachment point to the sense strand or the antisense strand, or to a conjugate linker conjugated to the sense strand or the antisense strand.

Embodiment 44: The dsRNAi agent of Embodiment 43, wherein the ligand comprises the following structure of

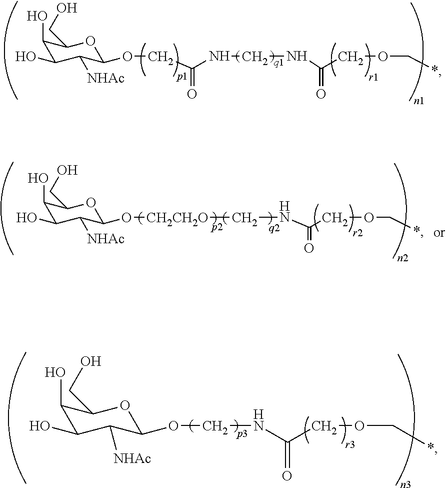

wherein:
each p1, p2, p3, q1, q2, r1, r2 and r3 is independently an integer from 0 to 12;
each n1, n2, and n3 is independently an integer from 1 to 3; and
“*” is an attachment point to L².

Embodiment 45: The dsRNAi agent of Embodiment 42, wherein the ligand has a structure of:

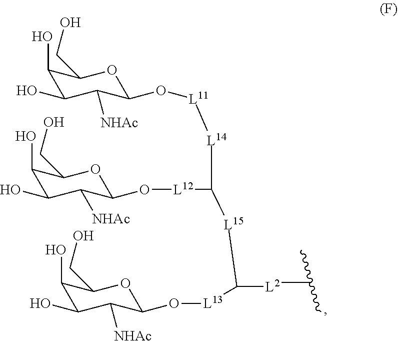

wherein:
each L¹¹, L¹², L¹³, L¹⁴, and L¹⁵ is an independently a linker;
L² is a linker;
[symbol] is an attachment point to the sense strand or the antisense strand, or to a conjugate linker conjugated to the sense strand or the antisense strand.

Embodiment 46: The dsRNAi agent of Embodiment 45, wherein the ligand has a structure of:

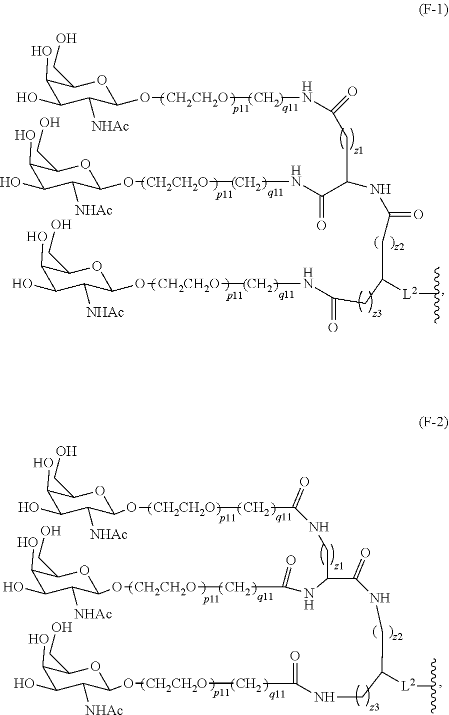

wherein:
each p11 and q11 is independently an integer from 0 to 12;
each z1, z2, and z3 is independently an integer of 0 to 12; and
[symbol] is an attachment point to the sense strand or the antisense strand, or to a conjugate linker conjugated to the sense strand or the antisense strand

Embodiment 47: The dsRNAi agent of any one of Embodiments 1 to 46, wherein the ligand comprises the following structure:

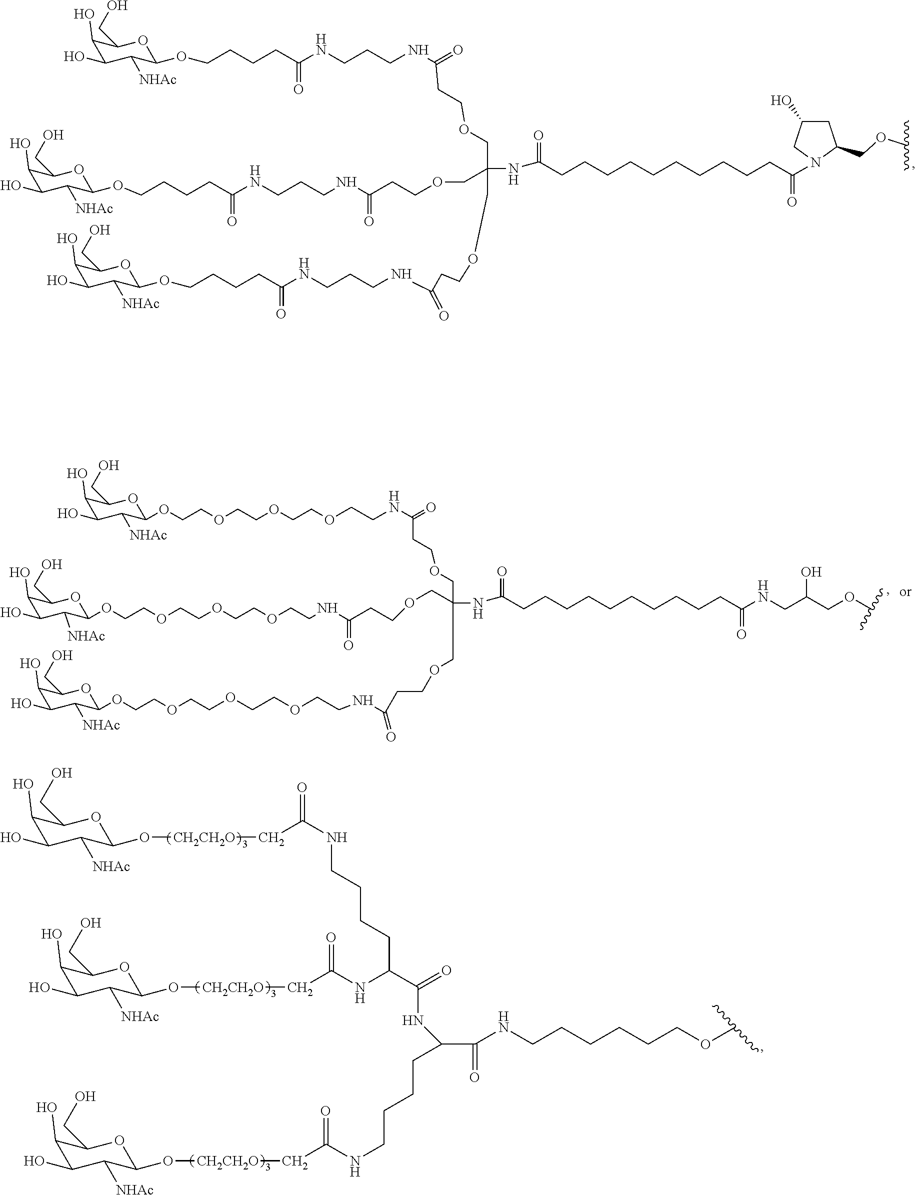

wherein:
[symbol] is an attachment point to the sense strand or the antisense strand or to a conjugate linker conjugated to the sense strand or the antisense strand.

Embodiment 48: The dsRNAi agent of Embodiment 47, wherein the ligand is conjugated to 3′ end of the sense strand to form the following structure:

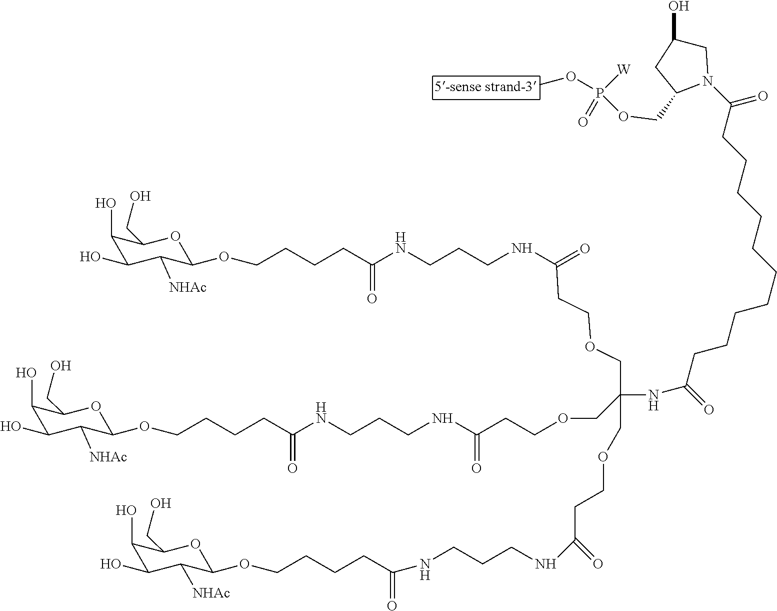

wherein W is —OH or —SH.

Embodiment 49: The dsRNAi agent of Embodiment 47, wherein the ligand is conjugated to 5′ end of the sense strand to form the following structure:

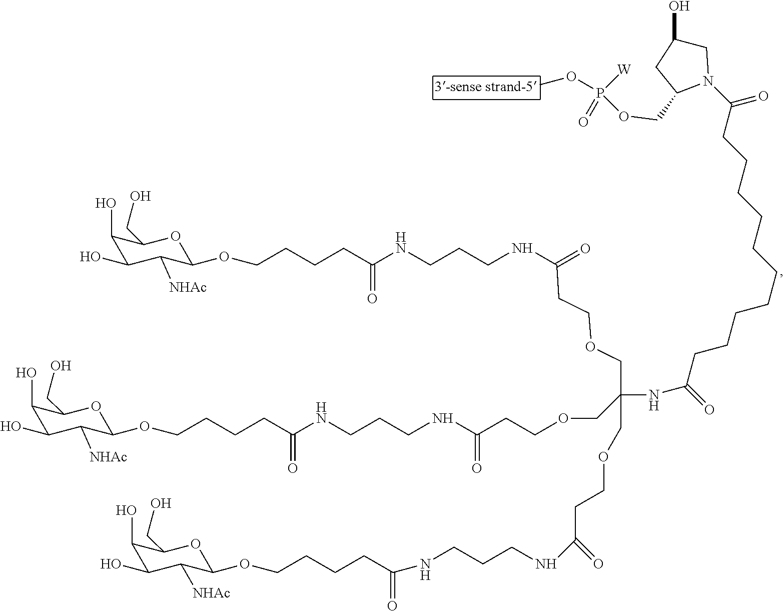

or a pharmaceutically acceptable salt thereof, wherein W is —OH or —SH.

Embodiment 50: The dsRNAi agent of Embodiment 48 or 49, wherein W is —OH.

Embodiment 51: A double stranded RNAi (dsRNAi) agent comprising:
(a) a sense strand consisting of SEQ ID NO: 906, and
   an antisense strand consisting of SEQ ID NO: 963;
(b) a sense strand consisting of SEQ ID NO: 907, and
   an antisense strand consisting of SEQ ID NO: 964;
(c) a sense strand consisting of SEQ ID NO: 947, and
   an antisense strand consisting of SEQ ID NO: 1004;
(d) a sense strand consisting of SEQ ID NO: 948, and
   an antisense strand consisting of SEQ ID NO: 1005;
(e) a sense strand consisting of SEQ ID NO: 949, and
   an antisense strand consisting of SEQ ID NO: 1006;
or
(f) a sense strand consisting of SEQ ID NO: 950, and
   an antisense strand consisting of SEQ ID NO: 1007;
wherein the ligand (L96) is conjugated to the 3′ end of the sense strand to form the following schematic:

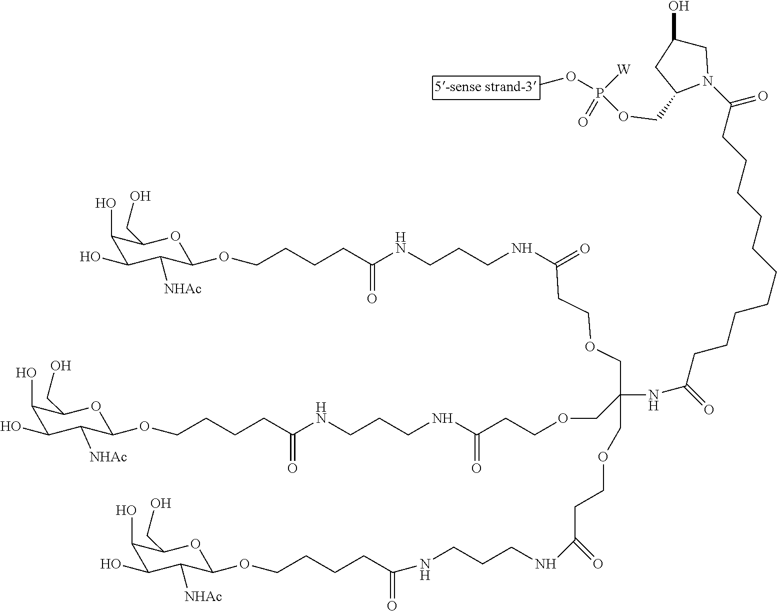

or a pharmaceutically acceptable salt thereof, wherein W is —OH.

Embodiment 52: The dsRNAi agent of any one of Embodiments 40 to 51, wherein the dsRNAi agent is in a pharmaceutically acceptable salt form.

Embodiment 53: The dsRNAi agent of Embodiment 52, wherein the pharmaceutically acceptable salt is a sodium salt.

Embodiment 54: A pharmaceutical composition comprising the dsRNAi agent of any one of Embodiments 1 to 53 or a pharmaceutically acceptable salt thereof, and a pharmaceutically acceptable carrier.

Embodiment 55: The pharmaceutical composition of Embodiment 54, wherein the composition is in an aqueous solution form.

Embodiment 56: A method of inhibiting PCSK9 expression in a cell, the method comprising:
(a) contacting the cell with the dsRNAi agent of any one of Embodiments 1 to 53 or a pharmaceutically acceptable salt thereof, or the pharmaceutical composition of any one of Embodiments 54 to 55; and
(b) maintaining the cell produced in step (a) for a time sufficient to obtain degradation of the mRNA transcript of a PCSK9 gene, thereby inhibiting expression of the PCSK9 gene in the cell.

Embodiment 57: A method of lowering a level of low-density lipoprotein cholesterol (LDL-C) in a subject in need thereof, comprising administering to the subject the dsRNAi agent of any one of Embodiments 1 to 53 or a pharmaceutically acceptable salt thereof, or the pharmaceutical composition of any one of Embodiments 54 to 55.

Embodiment 58: A method of treating lipidemia mediated by PCSK9 expression in a subject in need thereof, comprising administering to the subject the dsRNAi agent of any one of Embodiments 1 to 53 or a pharmaceutically acceptable salt thereof, or the pharmaceutical composition of any one of Embodiments 54 to 55.

Embodiment 59: A method of treating or preventing atherosclerotic cardiovascular disease (ASCVD) in a subject in need thereof, comprising administering to the subject the dsRNAi agent of any one of Embodiments 1 to 53 or a pharmaceutically acceptable salt thereof, or the pharmaceutical composition of any one of Embodiments 54 to 55.

Embodiment 60: A method of reducing or preventing cardiovascular event in a subject in need thereof, comprising administering to the subject the dsRNAi agent of any one of Embodiments 1 to 53 or a pharmaceutically acceptable salt thereof, or the pharmaceutical composition of any one of Embodiments 54 to 55.

Embodiment 61: The method of Embodiment 60, wherein the cardiovascular event is cardiovascular death, non-fatal myocardial infarction (MI), non-fatal ischemic stroke, urgent coronary revascularization, coronary heart disease (CHD) death, or any combination thereof.

Embodiment 62: A method of reducing or preventing a major limb adverse event (MALE) in a subject in need thereof, comprising administering to the subject the dsRNAi agent of any one of Embodiments 1 to 53 or a pharmaceutically acceptable salt thereof, or the pharmaceutical composition of any one of Embodiments 54 to 55.

Embodiment 63: The method of Embodiment 62, wherein the MALE is acute lower limb ischemia, lower limb amputation due to ischemia, urgent lower limb revascularization for ischemia, or any combination thereof.

Embodiment 64: A method of inhibiting PCSK9 expression in a cell, the method comprising:
(i) contacting the cell with a dsRNAi agent, wherein the dsRNAi agent comprises:
   (a) a sense strand comprising SEQ ID NO: 800, and
      an antisense strand comprising SEQ ID NO: 853;
   (b) a sense strand comprising SEQ ID NO: 801, and
      an antisense strand comprising SEQ ID NO: 854;
   (c) a sense strand comprising SEQ ID NO: 806, and
      an antisense strand comprising SEQ ID NO: 859;
   (d) a sense strand comprising SEQ ID NO: 811, and
      an antisense strand comprising SEQ ID NO: 864;
   (e) a sense strand comprising SEQ ID NO: 813, and
      an antisense strand comprising SEQ ID NO: 866;
   or
   (f) a sense strand comprising SEQ ID NO: 830, and
      an antisense strand comprising SEQ ID NO: 883,
(ii) maintaining the cell produced in step (a) for a time sufficient to obtain degradation of the mRNA transcript of a PCSK9 gene, thereby inhibiting expression of the PCSK9 gene in the cell.

Embodiment 65: A method of lowering a level of low-density lipoprotein cholesterol (LDL-C) in a subject in need thereof, comprising administering to the subject a dsRNAi agent, wherein the dsRNAi agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

Embodiment 66: A method of treating lipidemia mediated by PCSK9 expression in a subject in need thereof, comprising administering to the subject a dsRNAi agent, wherein the dsRNAi agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

Embodiment 67: A method of treating or preventing atherosclerotic cardiovascular disease (ASCVD) in a subject in need thereof, comprising administering to the subject a dsRNAi agent, wherein the dsRNAi agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

Embodiment 68: A method of reducing or preventing cardiovascular event in a subject in need thereof, comprising administering to the subject a dsRNAi agent, wherein the dsRNAi agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

Embodiment 69: The method of Embodiment 68, wherein the cardiovascular event is cardiovascular death, non-fatal myocardial infarction (MI), non-fatal ischemic stroke, urgent coronary revascularization, coronary heart disease (CHD) death, or any combination thereof.

Embodiment 70: A method of reducing or preventing a major limb adverse event (MALE) in a subject in need thereof, comprising administering to the subject a dsRNAi agent, wherein the dsRNAi agent comprises:
(a) a sense strand comprising SEQ ID NO: 800, and
   an antisense strand comprising SEQ ID NO: 853;
(b) a sense strand comprising SEQ ID NO: 801, and
   an antisense strand comprising SEQ ID NO: 854;
(c) a sense strand comprising SEQ ID NO: 806, and
   an antisense strand comprising SEQ ID NO: 859;
(d) a sense strand comprising SEQ ID NO: 811, and
   an antisense strand comprising SEQ ID NO: 864;
(e) a sense strand comprising SEQ ID NO: 813, and
   an antisense strand comprising SEQ ID NO: 866;
or
(f) a sense strand comprising SEQ ID NO: 830, and
   an antisense strand comprising SEQ ID NO: 883.

Embodiment 71: The method of Embodiment 70, wherein the MALE is acute lower limb ischemia, lower limb amputation due to ischemia, urgent lower limb revascularization for ischemia, or any combination thereof.

Embodiment 72: The method of any one of Embodiments 64 to 71, wherein the dsRNAi agent further comprises a ligand comprising the following structure:

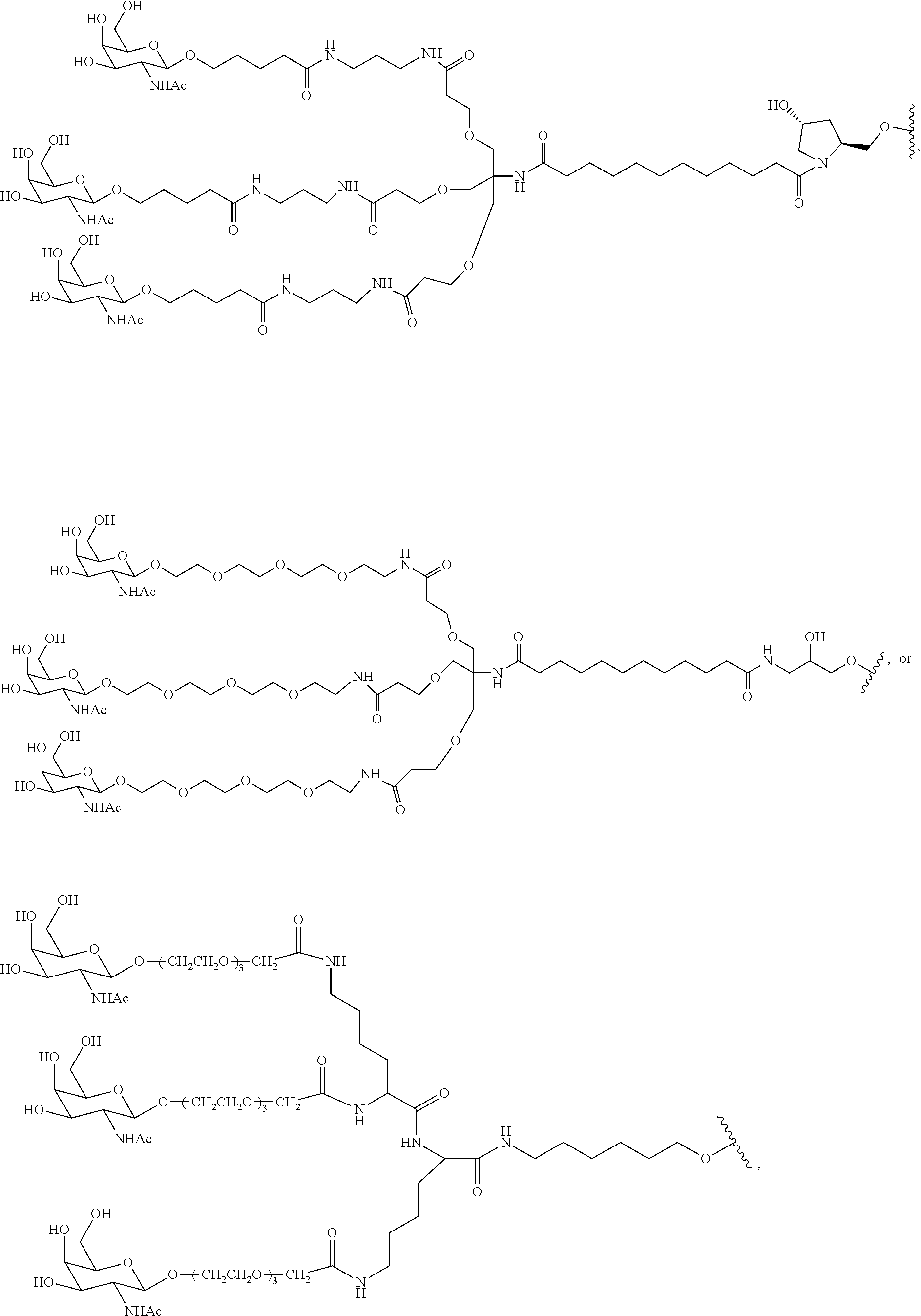

wherein
[symbol] is an attachment point to the sense strand or the antisense strand or to a conjugate linker conjugated to the sense strand or the antisense strand.

Embodiment 73: The method of any one of Embodiments 57 to 72, wherein the subject is a human.

Embodiment 74: The method of any one of Embodiments 57 to 72, wherein the subject has or is diagnosed with hyperlipidemia, hypercholesterolemia, hypertriglyceridemia, mixed hyperlipidemia, congestive heart disease (CHD) or atherosclerosis.

Embodiment 75: The method of any one of Embodiments 57 to 74, wherein the dsRNAi agent or the pharmaceutical composition is administered to the subject subcutaneously or intravenously.

Embodiment 76: A kit comprising the dsRNAi agent of any one of Embodiments 1 to 53 or a pharmaceutically acceptable salt thereof, or the pharmaceutical composition of any one of Embodiments 54 to 55.

Embodiment 77: The kit of Embodiment 76, further comprising an applicator.

Embodiment 78: The kit of Embodiment 77, wherein the applicator is a syringe.

Embodiment 79: The kit of Embodiment 78, wherein the applicator is a pre-filled syringe.

### EXAMPLES

### Example 1. Preparation of siRNAs

Small scale synthesis was used to prepare PCSK9 siRNAs; medium and large scale syntheses can also be used to prepare these siRNAs in larger quantities.

### Small Scale Synthesis and Purification Methods for the Initial Screens (1 μMole Scale)

Small scale synthesis was used to generate siRNAs. PCSK9 sequences (e.g., Tables 1-2) were synthesized on MerMade 192 synthesizer (BioAutomation, Plano, Tex.) at 1 □mol scale.

All oligonucleotides were prepared at 1 □mole scale using a MerMade 192 high-throughput synthesizer and commercially available phosphoramidite monomers, following standard protocols for solid-phase synthesis and deprotection. The GalNAc ligand was introduced at the 3′ end of the sense strand of the siRNA using a functionalized solid support, as previously described. PS linkages were prepared by oxidation of phosphite utilizing 0.1 M 3-((N,N-dimethyl-aminomethylidene)amino)-3H-1, 2, 4-dithiazole-5-thione (DDTT) in pyridine. After cleavage, deprotection, and precipitation of the products, each crude solution was desalted via size exclusion using water to elute the final oligonucleotide products. The identities and purities of all oligonucleotides were confirmed using electrospray ionization mass spectrometry (ESI-MS) and ion exchange-high-performance liquid chromatography (IEX-HPLC), respectively, and equimolar amounts of the complementary strands were annealed to provide the desired siRNA duplex. All duplexes met a purity cutoff of at least 85%.

### Synthesis, Cleavage and Deprotection:

The synthesis of PCSK9 sequences can use solid supported oligonucleotide synthesis using phosphoramidite chemistry.

The synthesis of the above sequences was performed at 1 QM scale in 96 well plates. The RNA, DNA, 2′-OMe, TNA, GNA, 5′-(E)-VP-2′-OMe, 2′-MOE, and 2′-F phosphoramidite solutions were prepared at 0.1 M concentration and 5-(ethylthio)tetrazole (0.25 M Acetonitrile) was used as activator. Deblocking solution, oxidizer solution and capping solution were prepared according to standard processes.

The synthesized sequences were cleaved and deprotected in 96 well plates, using methylamine solution (a 3:1 mixture of aqueous and ethanolic solutions) in the first step and fluoride reagent in the second step. The crude sequences were precipitated using acetone:ethanol (80:20) mix and the pellet were re-suspended in 0.02M sodium acetate buffer. Samples from each sequence were analyzed by LC-MS to confirm the identity, UV for quantification and a selected set of samples by IEX chromatography to determine purity.

### Purification and Desalting

PCSK9 tiled sequences were purified on AKTA explorer purification system using Source 15Q column. A column temperature of 65° C. was maintained during purification. Sample injection and collection were performed in 96 well (1.8 mL-deep well) plates. A single peak corresponding to the full length sequence was collected in the eluent. The purified sequences were desalted on a Sephadex G25 column using AKTA purifier. The concentration of desalted PCSK9 sequences were calculated using absorbance at 260 nm wavelength and purity was measured by ion exchange chromatography.

### Annealing

Purified desalted sense and antisense single strands were mixed in equimolar amounts and annealed to form PCSK9 duplexes. The duplexes were prepared at 10 uM concentration in 1×PBS buffer and tested by capillary gel electrophoresis for purity.

### Medium Scale Synthesis and Purification (1-50 μMol)

Medium scale synthesis can also be used to generate siRNAs. Single-stranded RNAs in scales between 1 and 50 μmol were prepared by solid phase synthesis using an MerMade 12 synthesizer (BioAutomation, Plano, Tex.). Universal Support was purchased from AM Chemicals LLC (VisTa, CA) and 3′-GalNAc controlled pore glass (CPG) support (500 Å, loading 50-100 μmol/g) were homemade. For larger scales, empty synthesis columns (10 μmol) from Glen Research Corp. and large amidite (250 mL) and reagent bottles (2000 mL) were used. RNA and RNA containing 2′-MOE, 2′-F or 2′-O-methyl nucleotides were generated by solid phase synthesis employing the corresponding phosphoramidites (Hongene Biotech Corporation, Union City, CA). These building blocks were incorporated at selected sites within the sequence of the oligoribonucleotide chain using standard nucleoside phosphoramidite chemistry such as described in Current Protocols in Nucleic Acid Chemistry, Beaucage, S. L. et al. (Edrs.), John Wiley & Sons, Inc., New York, NY, USA. Vinylphosphonates at the 5′ end of the antisense strand were introduced by using solid-phase synthesis of 5′(E)-vinylphosphonate-2′-OMe-U phosphoramidite monomers. Phosphorothioate linkages were introduced using a solution of the 0.1 M DDTT (AM Chemicals, Oceanside, CA) in pyridine.

The synthesized PCSK9 sequences were cleaved and deprotected in AMA solution (1:1 mixture of methylamine solution and 40% NH3 aqueous solutions) for 3.5 hours or in 40% NH3 aqueous solutions at 55° C. overnight. To deprotect 5′-Vinylphosphonates oligonucleotide, additional 3% of diethyl amine was add to deprotection solution. Preparative ion-pair reverse phase high-performance liquid chromatography (IPRP-HPLC) and IEX-HPLC were applied to purified oligonucleotide products. Samples from each sequence were analyzed by LC-MS to confirm the identity, UV absorbance at 260 nm for quantification and a selected set of samples by IPRP-HPLC and IEX-HPLC to determine purity. The identities and purities of all oligonucleotides were confirmed using electrospray ionization mass spectrometry (ESI-MS), Double stranded RNA was generated by mixing an equimolar solution of complementary strands in water or annealing buffer (typically phosphate buffered solution, PBS, Ambion, Applied Biosystems, Austin, TX) at the desired concentration. The mixture was then heated in a water bath at 85-90° C. for 5 minutes and cooled to room temperature over a period of 1-4 hours. The RNA duplex was stored at −20° C. until use.

A series siRNA duplexes were designed and synthesized using the techniques described above.

### Example 2. In Vitro Screening Profiling

siRNA sequences were designed to be complimentary to human PCSK9 transcript variant 1 (GenBank: NM_174936.4) and a few hundreds of RNAi agents (siRNAs) to PCSK9 were screened and tested for off-target hybridization (e.g., less off-target hybridization) and knock-down of PCSK9 mRNA in a cell.

PCSK9 siRNAs having the nucleotide sequences of U1 to U379 and modification patterns of SS1 (Table 3) and AS1 (Table 4) were synthesized as described above. Cells were transfected with 10 nM or 1 nM siRNA and the amount of PCSK9 mRNA remaining after 24 hours (expressed as PCSK9 mRNA/GAPDH mRNA ratio as percent of control cells) is indicated.

Table 10 below shows exemplary siRNA sequences tested for in vitro screening and resulting knockdown.

TABLE 10

| | | | 10 nM | 1 nM |
|---|---|---|---|---|
| | SS | AS | hsPCSK9/ | hsPCSK9/ |
| | modifi- | modifi- | hsGAPDH | hsGAPDH |
| PCSK9 | cation | cation | (% of | (% of |
| siRNA | pattern | pattern | control) | control) |
| U1 | SS1 | AS1 | 79 | 95 |
| U2 | SS1 | AS1 | 78 | 120 |
| U3 | SS1 | AS1 | 79 | 112 |
| U4 | SS1 | AS1 | 79 | 128 |
| U5 | SS1 | AS1 | 87 | 116 |
| U6 | SS1 | AS1 | 48 | 84 |
| U7 | SS1 | AS1 | 67 | 106 |
| U8 | SS1 | AS1 | 77 | 91 |
| U9 | SS1 | AS1 | 65 | 88 |
| U10 | SS1 | AS1 | 36 | 68 |
| U11 | SS1 | AS1 | 67 | 109 |
| U12 | SS1 | AS1 | 37 | 99 |
| U13 | SS1 | AS1 | 38 | 88 |
| U14 | SS1 | AS1 | 21 | 54 |
| U15 | SS1 | AS1 | 81 | 121 |
| U16 | SS1 | AS1 | 41 | 51 |
| U17 | SS1 | AS1 | 56 | 67 |
| U18 | SS1 | AS1 | 79 | 63 |
| U19 | SS1 | AS1 | 68 | 68 |
| U20 | SS1 | AS1 | 83 | 56 |
| U21 | SS1 | AS1 | 43 | 64 |
| U22 | SS1 | AS1 | 22 | 21 |
| U23 | SS1 | AS1 | 13 | 35 |
| U24 | SS1 | AS1 | 21 | 32 |
| U25 | SS1 | AS1 | 34 | 72 |
| U26 | SS1 | AS1 | 15 | 30 |
| U27 | SS1 | AS1 | 36 | 81 |
| U28 | SS1 | AS1 | 51 | 90 |
| U29 | SS1 | AS1 | 32 | 66 |
| U30 | SS1 | AS1 | 26 | 67 |
| U31 | SS1 | AS1 | 109 | 92 |
| U32 | SS1 | AS1 | 43 | 80 |
| U33 | SS1 | AS1 | 94 | 109 |
| U34 | SS1 | AS1 | 118 | 96 |
| U35 | SS1 | AS1 | 110 | 97 |
| U36 | SS1 | AS1 | 136 | 100 |
| U37 | SS1 | AS1 | 52 | 93 |
| U38 | SS1 | AS1 | 95 | 106 |
| U39 | SS1 | AS1 | 32 | 63 |
| U40 | SS1 | AS1 | 40 | 69 |
| U41 | SS1 | AS1 | 38 | 82 |
| U42 | SS1 | AS1 | 32 | 75 |
| U43 | SS1 | AS1 | 94 | 111 |
| U44 | SS1 | AS1 | 97 | 110 |
| U45 | SS1 | AS1 | 101 | 113 |
| U46 | SS1 | AS1 | 97 | 111 |
| U47 | SS1 | AS1 | 66 | 103 |
| U48 | SS1 | AS1 | 17 | 55 |
| U49 | SS1 | AS1 | 16 | 57 |
| U50 | SS1 | AS1 | 29 | 68 |
| U51 | SS1 | AS1 | 45 | 75 |
| U52 | SS1 | AS1 | 16 | 31 |
| U53 | SS1 | AS1 | 45 | 86 |
| U54 | SS1 | AS1 | 31 | 46 |
| U55 | SS1 | AS1 | 37 | 62 |
| U56 | SS1 | AS1 | 51 | 79 |
| U57 | SS1 | AS1 | 14 | 31 |
| U58 | SS1 | AS1 | 12 | 19 |
| U59 | SS1 | AS1 | 14 | 23 |
| U60 | SS1 | AS1 | 20 | 44 |
| U61 | SS1 | AS1 | 38 | 110 |
| U62 | SS1 | AS1 | 24 | 72 |
| U63 | SS1 | AS1 | 51 | 131 |
| U64 | SS1 | AS1 | 65 | 130 |
| U65 | SS1 | AS1 | 75 | 132 |
| U66 | SS1 | AS1 | 42 | 99 |
| U67 | SS1 | AS1 | 67 | 136 |
| U68 | SS1 | AS1 | 11 | 32 |
| U69 | SS1 | AS1 | 32 | 105 |
| U70 | SS1 | AS1 | 77 | 124 |
| U71 | SS1 | AS1 | 27 | 71 |
| U72 | SS1 | AS1 | 44 | 87 |
| U73 | SS1 | AS1 | 105 | 114 |
| U74 | SS1 | AS1 | 100 | 109 |
| U75 | SS1 | AS1 | 92 | 119 |
| U76 | SS1 | AS1 | 100 | 114 |
| U77 | SS1 | AS1 | 57 | 119 |
| U78 | SS1 | AS1 | 92 | 123 |
| U79 | SS1 | AS1 | 96 | 118 |
| U80 | SS1 | AS1 | 102 | 129 |
| U81 | SS1 | AS1 | 94 | 135 |
| U82 | SS1 | AS1 | 71 | 116 |
| U83 | SS1 | AS1 | 86 | 104 |
| U84 | SS1 | AS1 | 143 | 115 |
| U85 | SS1 | AS1 | 98 | 93 |
| U86 | SS1 | AS1 | 102 | 76 |
| U87 | SS1 | AS1 | 22 | 42 |
| U88 | SS1 | AS1 | 34 | 43 |
| U89 | SS1 | AS1 | 39 | 52 |
| U90 | SS1 | AS1 | 103 | 73 |
| U91 | SS1 | AS1 | 45 | 116 |
| U92 | SS1 | AS1 | 82 | 154 |
| U93 | SS1 | AS1 | 98 | 136 |
| U94 | SS1 | AS1 | 37 | 70 |
| U95 | SS1 | AS1 | 27 | 53 |
| U96 | SS1 | AS1 | 24 | 52 |
| U97 | SS1 | AS1 | 51 | 69 |
| U98 | SS1 | AS1 | 81 | 73 |
| U99 | SS1 | AS1 | 84 | 65 |
| U100 | SS1 | AS1 | 92 | 69 |
| U101 | SS1 | AS1 | 51 | 86 |
| U102 | SS1 | AS1 | 17 | 45 |
| U103 | SS1 | AS1 | 87 | 81 |
| U104 | SS1 | AS1 | 74 | 72 |
| U105 | SS1 | AS1 | 29 | 40 |
| U106 | SS1 | AS1 | 109 | 66 |
| U107 | SS1 | AS1 | 113 | 63 |
| U108 | SS1 | AS1 | 57 | 45 |
| U109 | SS1 | AS1 | 76 | 53 |
| U110 | SS1 | AS1 | 22 | 14 |
| U111 | SS1 | AS1 | 104 | 108 |
| U112 | SS1 | AS1 | 81 | 110 |
| U113 | SS1 | AS1 | 117 | 99 |
| U114 | SS1 | AS1 | 78 | 76 |
| U115 | SS1 | AS1 | 112 | 74 |
| U116 | SS1 | AS1 | 115 | 70 |
| U117 | SS1 | AS1 | 34 | 29 |
| U118 | SS1 | AS1 | 47 | 51 |
| U119 | SS1 | AS1 | 60 | 56 |
| U120 | SS1 | AS1 | 99 | 55 |
| U121 | SS1 | AS1 | 42 | 87 |
| U122 | SS1 | AS1 | 68 | 87 |
| U123 | SS1 | AS1 | 81 | 82 |
| U124 | SS1 | AS1 | 65 | 79 |
| U125 | SS1 | AS1 | 57 | 92 |
| U126 | SS1 | AS1 | 79 | 77 |
| U127 | SS1 | AS1 | 61 | 71 |
| U128 | SS1 | AS1 | 72 | 83 |
| U129 | SS1 | AS1 | 89 | 77 |
| U130 | SS1 | AS1 | 87 | 80 |
| U131 | SS1 | AS1 | 54 | 83 |
| U132 | SS1 | AS1 | 68 | 87 |
| U133 | SS1 | AS1 | 49 | 67 |
| U134 | SS1 | AS1 | 42 | 62 |
| U135 | SS1 | AS1 | 68 | 74 |
| U136 | SS1 | AS1 | 66 | 75 |
| U137 | SS1 | AS1 | 60 | 72 |
| U138 | SS1 | AS1 | 49 | 81 |
| U139 | SS1 | AS1 | 54 | 72 |
| U140 | SS1 | AS1 | 10 | 16 |
| U141 | SS1 | AS1 | 11 | 24 |
| U142 | SS1 | AS1 | 24 | 39 |
| U143 | SS1 | AS1 | 23 | 54 |
| U144 | SS1 | AS1 | 18 | 44 |
| U145 | SS1 | AS1 | 14 | 30 |
| U146 | SS1 | AS1 | 14 | 37 |
| U147 | SS1 | AS1 | 32 | 63 |
| U148 | SS1 | AS1 | 74 | 77 |
| U149 | SS1 | AS1 | 87 | 69 |
| U150 | SS1 | AS1 | 94 | 84 |
| U151 | SS1 | AS1 | 89 | 86 |
| U152 | SS1 | AS1 | 79 | 108 |
| U153 | SS1 | AS1 | 73 | 79 |
| U154 | SS1 | AS1 | 70 | 90 |
| U155 | SS1 | AS1 | 73 | 92 |
| U156 | SS1 | AS1 | 69 | 118 |
| U157 | SS1 | AS1 | 70 | 88 |
| U158 | SS1 | AS1 | 69 | 100 |
| U159 | SS1 | AS1 | 75 | 85 |
| U160 | SS1 | AS1 | 75 | 87 |
| U161 | SS1 | AS1 | 78 | 84 |
| U162 | SS1 | AS1 | 62 | 99 |
| U163 | SS1 | AS1 | 52 | 87 |
| U164 | SS1 | AS1 | 62 | 81 |
| U165 | SS1 | AS1 | 54 | 97 |
| U166 | SS1 | AS1 | 71 | 96 |
| U167 | SS1 | AS1 | 68 | 94 |
| U168 | SS1 | AS1 | 86 | 98 |
| U169 | SS1 | AS1 | 57 | 88 |
| U170 | SS1 | AS1 | 34 | 68 |
| U171 | SS1 | AS1 | 78 | 75 |
| U172 | SS1 | AS1 | 76 | 116 |
| U173 | SS1 | AS1 | 63 | 102 |
| U174 | SS1 | AS1 | 72 | 80 |
| U175 | SS1 | AS1 | 32 | 77 |
| U176 | SS1 | AS1 | 39 | 83 |
| U177 | SS1 | AS1 | 52 | 75 |
| U178 | SS1 | AS1 | 52 | 73 |
| U179 | SS1 | AS1 | 62 | 78 |
| U180 | SS1 | AS1 | 68 | 88 |
| U181 | SS1 | AS1 | 80 | 72 |
| U182 | SS1 | AS1 | 63 | 96 |
| U183 | SS1 | AS1 | 23 | 80 |
| U184 | SS1 | AS1 | 66 | 94 |
| U185 | SS1 | AS1 | 67 | 99 |
| U186 | SS1 | AS1 | 38 | 86 |
| U187 | SS1 | AS1 | 22 | 70 |
| U188 | SS1 | AS1 | 76 | 91 |
| U189 | SS1 | AS1 | 58 | 89 |
| U190 | SS1 | AS1 | 31 | 61 |
| U191 | SS1 | AS1 | 26 | 64 |
| U192 | SS1 | AS1 | 72 | 83 |
| U193 | SS1 | AS1 | 20 | 38 |
| U194 | SS1 | AS1 | 51 | 83 |
| U195 | SS1 | AS1 | 48 | 77 |
| U196 | SS1 | AS1 | 69 | 87 |
| U197 | SS1 | AS1 | 74 | 94 |
| U198 | SS1 | AS1 | 50 | 93 |
| U199 | SS1 | AS1 | 60 | 90 |
| U200 | SS1 | AS1 | 82 | 98 |
| U201 | SS1 | AS1 | 69 | 86 |
| U202 | SS1 | AS1 | 22 | 60 |
| U203 | SS1 | AS1 | 35 | 83 |
| U204 | SS1 | AS1 | 85 | 99 |
| U205 | SS1 | AS1 | 82 | 97 |
| U206 | SS1 | AS1 | 78 | 88 |
| U207 | SS1 | AS1 | 64 | 93 |
| U208 | SS1 | AS1 | 39 | 67 |
| U209 | SS1 | AS1 | 13 | 31 |
| U210 | SS1 | AS1 | 24 | 45 |
| U211 | SS1 | AS1 | 30 | 71 |
| U212 | SS1 | AS1 | 38 | 81 |
| U213 | SS1 | AS1 | 43 | 77 |
| U214 | SS1 | AS1 | 89 | 96 |
| U215 | SS1 | AS1 | 72 | 99 |
| U216 | SS1 | AS1 | 39 | 89 |
| U217 | SS1 | AS1 | 66 | 94 |
| U218 | SS1 | AS1 | 52 | 98 |
| U219 | SS1 | AS1 | 73 | 101 |
| U220 | SS1 | AS1 | 90 | 117 |
| U221 | SS1 | AS1 | 102 | 101 |
| U222 | SS1 | AS1 | 91 | 82 |
| U223 | SS1 | AS1 | 92 | 73 |
| U224 | SS1 | AS1 | 86 | 72 |
| U225 | SS1 | AS1 | 87 | 79 |
| U226 | SS1 | AS1 | 75 | 80 |
| U227 | SS1 | AS1 | 85 | 76 |
| U228 | SS1 | AS1 | 87 | 88 |
| U229 | SS1 | AS1 | 73 | 78 |
| U230 | SS1 | AS1 | 51 | 68 |
| U231 | SS1 | AS1 | 80 | 99 |
| U232 | SS1 | AS1 | 29 | 43 |
| U233 | SS1 | AS1 | 104 | 85 |
| U234 | SS1 | AS1 | 86 | 85 |
| U235 | SS1 | AS1 | 87 | 88 |
| U236 | SS1 | AS1 | 86 | 95 |
| U237 | SS1 | AS1 | 88 | 93 |
| U238 | SS1 | AS1 | 85 | 87 |
| U239 | SS1 | AS1 | 52 | 88 |
| U240 | SS1 | AS1 | 66 | 88 |
| U241 | SS1 | AS1 | 80 | 92 |
| U242 | SS1 | AS1 | 82 | 97 |
| U243 | SS1 | AS1 | 79 | 88 |
| U244 | SS1 | AS1 | 16 | 43 |
| U245 | SS1 | AS1 | 57 | 82 |
| U246 | SS1 | AS1 | 33 | 69 |
| U247 | SS1 | AS1 | 44 | 87 |
| U248 | SS1 | AS1 | 41 | 83 |
| U249 | SS1 | AS1 | 96 | 102 |
| U250 | SS1 | AS1 | 98 | 97 |
| U251 | SS1 | AS1 | 75 | 81 |
| U252 | SS1 | AS1 | 65 | 82 |
| U253 | SS1 | AS1 | 84 | 85 |
| U254 | SS1 | AS1 | 70 | 88 |
| U255 | SS1 | AS1 | 57 | 87 |
| U256 | SS1 | AS1 | 77 | 95 |
| U257 | SS1 | AS1 | 64 | 87 |
| U258 | SS1 | AS1 | 75 | 94 |
| U259 | SS1 | AS1 | 101 | 87 |
| U260 | SS1 | AS1 | 90 | 95 |
| U261 | SS1 | AS1 | 41 | 88 |
| U262 | SS1 | AS1 | 80 | 91 |
| U263 | SS1 | AS1 | 59 | 104 |
| U264 | SS1 | AS1 | 76 | 105 |
| U265 | SS1 | AS1 | 63 | 96 |
| U266 | SS1 | AS1 | 78 | 98 |
| U267 | SS1 | AS1 | 79 | 103 |
| U268 | SS1 | AS1 | 57 | 41 |
| U269 | SS1 | AS1 | 72 | 98 |
| U270 | SS1 | AS1 | 45 | 77 |
| U271 | SS1 | AS1 | 24 | 57 |
| U272 | SS1 | AS1 | 21 | 61 |
| U273 | SS1 | AS1 | 30 | 59 |
| U274 | SS1 | AS1 | 96 | 103 |
| U275 | SS1 | AS1 | 96 | 104 |
| U276 | SS1 | AS1 | 37 | 99 |
| U277 | SS1 | AS1 | 78 | 115 |
| U278 | SS1 | AS1 | 74 | 73 |
| U279 | SS1 | AS1 | 76 | 81 |
| U280 | SS1 | AS1 | 32 | 82 |
| U281 | SS1 | AS1 | 37 | 89 |
| U282 | SS1 | AS1 | 23 | 54 |
| U283 | SS1 | AS1 | 47 | 92 |
| U284 | SS1 | AS1 | 17 | 35 |
| U285 | SS1 | AS1 | 53 | 91 |
| U286 | SS1 | AS1 | 115 | 133 |
| U287 | SS1 | AS1 | 92 | 112 |
| U288 | SS1 | AS1 | 92 | 118 |
| U289 | SS1 | AS1 | 46 | 105 |
| U290 | SS1 | AS1 | 102 | 121 |
| U291 | SS1 | AS1 | 63 | 93 |
| U292 | SS1 | AS1 | 69 | 115 |
| U293 | SS1 | AS1 | 88 | 103 |
| U294 | SS1 | AS1 | 91 | 97 |
| U295 | SS1 | AS1 | 96 | 113 |
| U296 | SS1 | AS1 | 75 | 112 |
| U297 | SS1 | AS1 | 81 | 115 |
| U298 | SS1 | AS1 | 49 | 100 |
| U299 | SS1 | AS1 | 53 | 113 |
| U300 | SS1 | AS1 | 28 | 75 |
| U301 | SS1 | AS1 | 83 | 69 |
| U302 | SS1 | AS1 | 41 | 50 |
| U303 | SS1 | AS1 | 29 | 40 |
| U304 | SS1 | AS1 | 20 | 30 |
| U305 | SS1 | AS1 | 24 | 39 |
| U306 | SS1 | AS1 | 35 | 49 |
| U307 | SS1 | AS1 | 31 | 47 |
| U308 | SS1 | AS1 | 52 | 52 |
| U309 | SS1 | AS1 | 54 | 73 |
| U310 | SS1 | AS1 | 23 | 29 |
| U311 | SS1 | AS1 | 46 | 80 |
| U312 | SS1 | AS1 | 94 | 86 |
| U313 | SS1 | AS1 | 21 | 42 |
| U314 | SS1 | AS1 | 29 | 50 |
| U315 | SS1 | AS1 | 31 | 61 |
| U316 | SS1 | AS1 | 50 | 96 |
| U317 | SS1 | AS1 | 41 | 74 |
| U318 | SS1 | AS1 | 95 | 115 |
| U319 | SS1 | AS1 | 59 | 97 |
| U320 | SS1 | AS1 | 137 | 107 |
| U321 | SS1 | AS1 | 65 | 85 |
| U322 | SS1 | AS1 | 46 | 75 |
| U323 | SS1 | AS1 | 31 | 66 |
| U324 | SS1 | AS1 | 62 | 81 |
| U325 | SS1 | AS1 | 21 | 48 |
| U326 | SS1 | AS1 | 38 | 62 |
| U327 | SS1 | AS1 | 14 | 17 |
| U328 | SS1 | AS1 | 17 | 21 |
| U329 | SS1 | AS1 | 17 | 22 |
| U330 | SS1 | AS1 | 42 | 76 |
| U331 | SS1 | AS1 | 17 | 41 |
| U332 | SS1 | AS1 | 26 | 49 |
| U333 | SS1 | AS1 | 49 | 77 |
| U334 | SS1 | AS1 | 66 | 79 |
| U335 | SS1 | AS1 | 73 | 81 |
| U336 | SS1 | AS1 | 36 | 65 |
| U337 | SS1 | AS1 | 44 | 81 |
| U338 | SS1 | AS1 | 48 | 76 |
| U339 | SS1 | AS1 | 17 | 37 |
| U340 | SS1 | AS1 | 14 | 17 |
| U341 | SS1 | AS1 | 13 | 23 |
| U342 | SS1 | AS1 | 10 | 16 |
| U343 | SS1 | AS1 | 11 | 15 |
| U344 | SS1 | AS1 | 22 | 53 |
| U345 | SS1 | AS1 | 23 | 49 |
| U346 | SS1 | AS1 | 24 | 61 |
| U347 | SS1 | AS1 | 33 | 77 |
| U348 | SS1 | AS1 | 19 | 40 |
| U349 | SS1 | AS1 | 17 | 13 |
| U350 | SS1 | AS1 | 15 | 14 |
| U351 | SS1 | AS1 | 14 | 20 |
| U352 | SS1 | AS1 | 12 | 21 |
| U353 | SS1 | AS1 | 12 | 12 |
| U354 | SS1 | AS1 | 13 | 17 |
| U355 | SS1 | AS1 | 15 | 17 |
| U356 | SS1 | AS1 | 9 | 16 |
| U357 | SS1 | AS1 | 9 | 16 |
| U358 | SS1 | AS1 | 13 | 22 |
| U359 | SS1 | AS1 | 13 | 14 |
| U360 | SS1 | AS1 | 14 | 16 |
| U361 | SS1 | AS1 | 12 | 14 |
| U362 | SS1 | AS1 | 14 | 14 |
| U363 | SS1 | AS1 | 15 | 17 |
| U364 | SS1 | AS1 | 16 | 19 |
| U365 | SS1 | AS1 | 11 | 15 |
| U366 | SS1 | AS1 | 11 | 16 |
| U367 | SS1 | AS1 | 12 | 22 |
| U368 | SS1 | AS1 | 15 | 20 |
| U369 | SS1 | AS1 | 10 | 16 |
| U370 | SS1 | AS1 | 8 | 13 |
| U371 | SS1 | AS1 | 13 | 24 |
| U372 | SS1 | AS1 | 12 | 20 |
| U373 | SS1 | AS1 | 15 | 19 |
| U374 | SS1 | AS1 | 21 | 42 |
| U375 | SS1 | AS1 | 25 | 46 |
| U376 | SS1 | AS1 | 12 | 24 |
| U377 | SS1 | AS1 | 9 | 20 |
| U378 | SS1 | AS1 | 8 | 20 |
| U379 | SS1 | AS1 | 6 | 15 |

### Example 3. In Vitro Dose-Dependent Screening

### Dual Dose Screening in A431 Cells:

A431 cells (ATCC. Cat. CRL-1555) were cultured in DMEM (ATCC. Cat. 30-2002) medium containing 10% FBS at 37 C with 5% CO₂. Cells were seeded in a 96-well plate and reverse transfected using RNAiMax (Thermo. RNAiMax. Cat. 13778150) with either 10 nM or 1 nM of siRNA and 24 hrs post transfection knockdown of PCSK9 was determined by Branch DNA assay. The degree of knock-down as calculated from the ratio of PCSK9 signal to GAPDH signal.

### Dose Response Curves in A431 Cells

A431 cells (ATCC. Cat. CRL-1555) were cultured in DMEM (ATCC. Cat. 30-2002) medium containing 10% FBS at 37° C. with 5% CO2. Cells were seeded in a 96-well plate and reverse transfected using RNAiMax (Thermo. RNAiMax. Cat. 13778150) in a 10-step dose range starting with 25 nM using a 1:4 dilution factor. Remaining PCSK9 mRNA levels were measured 24 hrs post transfection by Branch DNA assay. The degree of knock-down as calculated from the ratio of PCSK9 signal to GAPDH signal. Degree of knock-down vs. siRNA concentration data were used to determine the IC50 and maximum inhibition (%).

PCSK9 siRNAs having the nucleotide sequences in Table 11 with modification patterns of SS1 (Table 3) and AS1 (Table 4) were synthesized as described above. The IC50 and maximum inhibition (%) values for each of these siRNAs are shown in Table 11.

TABLE 11

| Unmodified | SS | AS | | max. |
|---|---|---|---|---|
| PCSK9 | Modification | Modification | | inhib. |
| siRNA | pattern | pattern | IC₅₀ | [%] |
| U4 | SS1 | AS1 | 0.60 | 82.31 |
| U22 | SS1 | AS1 | 0.03 | 84.01 |
| U23 | SS1 | AS1 | 0.41 | 91.92 |
| U24 | SS1 | AS1 | 0.37 | 85.78 |
| P13 | SS1 | AS1 | 0.29 | 83.04 |
| U49 | SS1 | AS1 | 0.81 | 77.80 |
| U52 | SS1 | AS1 | 0.19 | 89.17 |
| U57 | SS1 | AS1 | 0.31 | 89.55 |
| P5 | SS1 | AS1 | 0.09 | 93.45 |
| U59 | SS1 | AS1 | 0.23 | 91.38 |
| U68 | SS1 | AS1 | 0.21 | 87.78 |
| U102 | SS1 | AS1 | 0.63 | 83.59 |
| P14 | SS1 | AS1 | 0.86 | 78.40 |
| U110 | SS1 | AS1 | 0.24 | 88.69 |
| U117 | SS1 | AS1 | 0.42 | 81.33 |
| U140 | SS1 | AS1 | 0.17 | 92.29 |
| U145 | SS1 | AS1 | 0.14 | 87.78 |
| U145 | SS1 | AS1 | 0.51 | 89.52 |
| U146 | SS1 | AS1 | 0.23 | 91.65 |
| U244 | SS1 | AS1 | 0.44 | 82.97 |
| U284 | SS1 | AS1 | 0.18 | 87.39 |
| U304 | SS1 | AS1 | 0.27 | 86.28 |
| P15 | SS1 | AS1 | 0.21 | 88.27 |
| U327 | SS1 | AS1 | 0.09 | 86.93 |
| U331 | SS1 | AS1 | 0.37 | 79.77 |
| P8 | SS1 | AS1 | 0.15 | 87.19 |
| U340 | SS1 | AS1 | 0.09 | 82.52 |
| U342 | SS1 | AS1 | 0.04 | 91.65 |
| U343 | SS1 | AS1 | 0.03 | 92.39 |
| U349 | SS1 | AS1 | 0.02 | 89.46 |
| U352 | SS1 | AS1 | 0.07 | 91.33 |
| U353 | SS1 | AS1 | 0.02 | 89.05 |
| U355 | SS1 | AS1 | 0.01 | 89.15 |
| U356 | SS1 | AS1 | 0.06 | 91.66 |
| U357 | SS1 | AS1 | 0.07 | 93.90 |
| U359 | SS1 | AS1 | 0.04 | 86.60 |
| U361 | SS1 | AS1 | 0.04 | 85.82 |
| U362 | SS1 | AS1 | 0.03 | 90.59 |
| U364 | SS1 | AS1 | 0.05 | 87.51 |
| U365 | SS1 | AS1 | 0.03 | 91.67 |
| U366 | SS1 | AS1 | 0.03 | 86.00 |
| U369 | SS1 | AS1 | 0.03 | 87.43 |
| U370 | SS1 | AS1 | 0.04 | 89.91 |
| U373 | SS1 | AS1 | 0.03 | 84.65 |
| U377 | SS1 | AS1 | 0.03 | 87.86 |
| U378 | SS1 | AS1 | 0.03 | 86.45 |
| U379 | SS1 | AS1 | 0.01 | 90.92 |
| P16 | SS1 | AS1 | 0.24 | 81.02 |

### Example 4. In Vivo Profiling in Mice with Humanized PCSK9

Human PCSK9 knockin/murine PCSK9 knockout mice (B6-huPCSK9-UTR mice) were obtained from GemPharmaTech. The mice (n=4 mice per group) were fed a standard chow diet. For PK/PD studies, mice were administered phosphate-buffered saline (PBS) vehicle or 3 mg/kg siRNA in PBS by subcutaneous (SC) injection. Plasma was prepared from blood samples obtained via tail-nick at baseline (pre-dose) and at multiple post-dose timepoints from non-fasted mice. At the study endpoint, mice were sacrificed and liver samples were obtained for assessment of human PCSK9 mRNA levels, liver tissue siRNA levels, and level of siRNA loading into the RNA-induced silencing complex (RISC).

### Plasma Human PCSK9 ELISA

PCSK9 protein concentrations in plasma samples were determined using a human PCSK9 ELISA kit from R&D Systems (catalog no. SPC900). Reagents and plasma samples were brought to room temperature before use. Plasma samples were diluted 1:2, 1:5 or 1:25 by adding 62.5 μL, 25 μL, or 5 μL, respectively, of plasma sample to 62.5 μL, 100 μL, or 120 μL, respectively, of 1× Calibrator Diluent and mixing by pipetting. Cynomolgus monkey PCSK9 standard protein was diluted to 40 ng/mL using Calibrator Diluent, then six serial 1:2 dilutions were made. To each well of a 96-well plate pre-coated with capture antibody, Assay Diluent was added (100 μL/well) followed by diluted plasma sample or standard (50 μL/well). All plasma samples and standards were run in duplicate on the same plate. The plate was then sealed and incubated at room temperature for 105 minutes on a plate shaker. After incubation, the plate was flipped upside down with force to remove liquid from the wells and then blotted with a paper towel to absorb remaining solution. Wash Buffer was then added to each well (400 μL/well) using a plate washer. The plate was then flipped with force to remove liquid from the wells and blotted with a paper towel. This step was repeated three times for a total of four washes. Next, detection antibody (Human PCSK9 Conjugate) was added to each well (200 μL/well). The plate was then sealed and incubated at room temperature for 105 minutes on a plate shaker. After incubation, the plate was washed four times, as described previously. Equal volumes of Color Reagent A and Color Reagent B were then added to a 50 mL conical tube and mixed via inversion to create the Substrate Solution. Substrate Solution was then added to each well (200 μL/well). The plate was then sealed, shielded from light and incubated at room temperature for 30 minutes on a plate shaker. After incubation, Stop Solution (50 μL) was added to each well and mixed by swirling pipette tips in the wells (without touching the bottom) until the color of the solution changed from blue to a uniform yellow color. The absorbance of each well at 450 nm was then measured using a Molecular Devices Spectramax M2 plate reader. The absorbance values for the standards were used to construct a standard curve. Plasma PCSK9 concentrations for each sample were then determined from the observed absorbance for that sample and the standard curve by interpolation, followed by adjustment for the 1:2, 1:5, or 1:25 sample dilution factor. Reported PCSK9 concentrations are the average values from two replicate wells from the same plate.

Liver Human PCSK9 mRNA Assessments

Liver RNA extraction was performed using a TRIZOL® RNA Isolation protocol followed by an Qiagen RNeasy MiniPrep protocol. Briefly, an approximately 50 mg piece of frozen liver was lysed and homogenized using a Tissue Lyser II (Qiagen, Germany) with one stainless steel bead and TRIZOL® lysis reagent (Invitrogen, Carlsbad CA). Next, chloroform was added, and phases were separated. RNA was precipitated using isopropyl alcohol and washed with 75% ethanol. RNA was bound, washed, and eluted from RNeasy mini spin columns (Qiagen, Germany). The optional on column DNase digestion was performed using the RNase free DNase set (Qiagen, Germany). A total of 100 μL of RNA was eluted from each sample. RNA samples were quantified using a BioTek Take3 Trio Microvolume Plate (Agilent, Santa Clara CA) in a BioTek Cytation 5 plate reader (Agilent, Santa Clara CA). Quantified RNA samples were diluted then reverse transcribed using TaqMan™ Reverse Transcription Reagents (ThermoFisher, Waltham MA). TaqMan™ qPCR was performed with the cDNA samples using TaqMan™ gene expression assays for mouse TATA binding protein (catalog #Mm00446971_m1) and human PCSK9 (catalog #Hs00545399_m1) according to the manufacturer's instructions (ThermoFisher, Waltham MA). The human PCSK9 level in each sample was normalized using that sample's level of the housekeeping gene TATA binding protein.

Liver siRNA Level Measurements

Tissue lysis: each frozen tissue piece (˜50 mg) was transferred to a 2 mL round bottom microcentrifuge tube that was pre-chilled on dry ice. One dry ice pre-chilled 5 mm stainless steel bead (Qiagen, Germany) was added to the tube containing the frozen tissue. In the cold room, sample tubes were quickly removed from dry ice and 1 mL of ice-cold Lysis buffer (50 mM Tris-HCl pH 7.5, 150 mM NaCl, 2 mM EDTA, 1 mM PMSF, 1× protease inhibitor, 0.5% Triton X-100)was added to each tube. Tissues were then immediately lysed using a TissueLyser LT (Qiagen, Germany) for 5 min at 50 Hz in cold room. The lysate was then cleared by centrifugation at 20000×g and 4° C. for 10 min, and the soluble lysate supernatants were kept on ice or stored at −80° C. for subsequent analysis.

Stem Loop-Reverse Transcription Quantitative polymerase chain reaction (SL-RT-qPCR): Liver lysates were diluted in PBST (PBS, 0.25% Triton X-100) at least 100-fold (typically 10000-fold) in PBST prior to the reverse transcription step. For the detection of siRNA via SL-RT-qPCR, primers were designed against the antisense strand sequence of interest using the design tool on the vendor website (Custom Taqman Small RNA Assay, 4398987, ThermoFisher, Waltham MA). Each custom Taqman primer set contains a SL-RT primer and Taqman qPCR primer specific to the antisense strand sequence of interest. cDNA was generated by following the manufacturer's protocol of the Taqman MicroRNA Reverse Transcription Kit (ThermoFisher, Waltham MA), using 5 μL diluted lysate in PBST or 5 μL siRNA standard. The cDNA generated (15 μL reaction) were then diluted with 15 μL water prior to the qPCR step. qPCR was performed following the manufacturer's protocol for TaqMan™ Fast Advanced Master Mix (ThermoFisher, Waltham MA), using 4 μL of the diluted cDNA.

Calculations: siRNA standards were prepared in PBST by 10-fold serial dilution, starting at 100 ng/mL down to 0.0001 ng/mL. Ct values obtained from each standard were plotted on (Y) vs siRNA concentration (X) in log scale in GraphPad Prism. A semilog line was fitted to determine the slope and y-intercept values. siRNA concentration (x) for each sample was then determined based on the obtained Ct value (y), and the determined slope (m) and y-intercept (b) values from the standard curve based on the equation y=mx+b. To calculate total liver siRNA (ng siRNA per g tissue), the following operations were performed: 1) Volume of total lysate=1000 mL Lysis buffer plus tissue volume (assuming 1 μL per mg of tissue sample), 2) ng siRNA=[siRNA]×Volume of total lysate×dilution factor, and 3) total liver siRNA (ng/g tissue)=ng siRNA/g sample tissue weight.

### Liver RISC Loading Experiments

Tissue lysis: each frozen tissue piece (˜50 mg) was transferred to a 2 mL round bottom microcentrifuge tube that was pre-chilled on dry ice. One dry ice pre-chilled 5 mm stainless steel bead (Qiagen, Germany) was added to the tube containing the frozen tissue. In the cold room, sample tubes were quickly removed from dry ice and 1 mL of ice-cold Lysis buffer (50 mM Tris-HCl pH 7.5, 150 mM NaCl, 2 mM EDTA, 1 mM PMSF, 1× protease inhibitor, 0.5% Triton X-100) was added to each tube. Tissues were then immediately lysed using a TissueLyser LT (Qiagen, Germany) for 5 min at 50 Hz in a cold room. Lysate was then cleared by centrifugation at 20000×g at 4° C. for 10 min, and the soluble lysate supernatants were kept on ice. The protein concentration of the soluble lysate for each sample was determined using a BCA assay (ThermoFisher, Waltham MA) according to manufacturer's protocol.

Ago2 immunoprecipitation (IP): for IP, 50 μL of Dynabead Protein G (ThermoFisher, Waltham MA) slurry was used per sample. Prior to use for IP, the beads were washed with at least 2× volume of Wash Buffer (150 mM NaCl, 2 mM EDTA, 0.5% Triton X-100), and then resuspended in 4× volume of Wash Buffer. Mouse Ago2 antibody (FUJFILM Wako Chemicals, Richmond VA) was pre-bound to the beads in Wash Buffer (200 ng antibody per 50 μL bead slurry) at 4° C. for 2 h on a rotating mixer. After incubation, the Ago2 antibody-bound beads were washed twice with 10 volumes of Wash buffer, and then resuspended in Lysis Buffer. Bead suspension equivalent to amount of 50 μL original bead slurry was distributed to a 1.5 mL microcentrifuge tube per sample. For each sample, Ago2 antibody-bound beads were incubated with 500 mg soluble tissue lysate in lysis buffer at final volume of 250 μL per sample at 4° C. overnight on a rotating mixer. After incubation, the beads in each tube were washed 5 times with 1 mL ice cold Wash Buffer. The beads were then resuspended in 50 mL of PBST per sample. siRNAs were released from the beads by heating at 95° C. for 5 min. Ago2 IP eluate supernatants were recovered and kept on ice for SL-RT-qPCR analysis on the same day, or stored at −80° C. for SL-RT-qPCR on a later day.

Stem Loop-Reverse Transcription Quantitative polymerase chain reaction (SL-RT-qPCR): For detection of siRNA via SL-RT-qPCR, primers were designed against the antisense strand sequence of interest using the design tool on the vendor website (Custom Taqman Small RNA Assay, 4398987, ThermoFisher, Waltham MA). Each custom Taqman primer set contains a SL-RT primer and Taqman qPCR primer specific to the antisense strand sequence of interest. cDNA was generated following manufacturer's protocol of the Taqman MicroRNA Reverse Transcription Kit (ThermoFisher, Waltham MA), using 5 mL of Ago2 IP eluate or 5 mL siRNA standard. The cDNA generated (15 μL reaction) were then diluted with 75 mL water prior to usage for the qPCR step. qPCR was performed following the manufacturer's protocol for TaqMan™ Fast Advanced Master Mix (ThermoFisher, Waltham MA), using 4 mL of the diluted cDNA.

Calculations: siRNA standards were prepared in PBST by 10-fold serial dilution, starting at 100 ng/mL down to 0.00001 ng/mL. Ct values obtained from each standard were plotted on (Y) vs siRNA concentration (X) in log scale in GraphPad Prism. A semilog line was fitted to determine the slope and y-intercept values. The siRNA concentration (x) for each sample was then determined based on the obtained Ct value (y), and the determined slope (m) and y-intercept (b) values from the standard curve based on the equation y=mx+b. To calculate ng siRNA loaded on RISC per g tissue, the following operations were performed: 1) ng siRNA=[siRNA]×Ago2 IP elution volume (50 mL), 2) [tissue lysate]=mg tissue per 1 mL lysis buffer, 3) g tissue used=[tissue lysate]×volume lysate used in Ago2 IP, and 4) RISC-loading (ng/g tissue)=ng siRNA/g tissue used.

Results of siRNA In Vivo Profiling

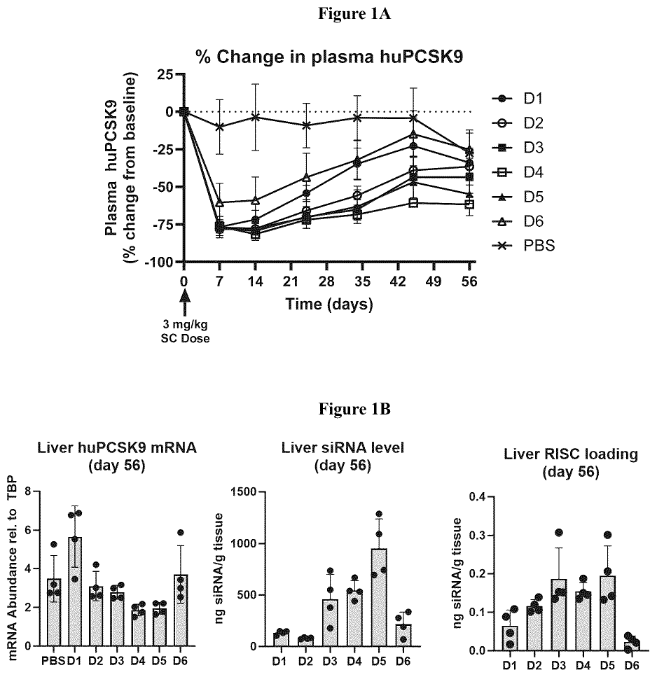

Leqvio® (inclisiran) is a small interfering ribonucleic acid (siRNA) targeting hepatic PCSK9 mRNA to lower plasma LDL-C levels. The dsRNAi agent D1 described herein includes the same sense and antisense strand sequences and chemical modifications as inclisiran, but a different GalNAc ligand. The pharmacodynamic (PD) and pharmacokinetic (PK) properties of a number of dsRNAi agents (D1-D6) described herein were evaluated in B6-huPCSK9-UTR mice administered PBS vehicle or 3 mg/kg siRNA in PBS via subcutaneous injection. As shown in FIG. 1A, administration of each of these siRNAs, with the exception of D6, resulted in a similar decrease of plasma PCSK9 from pre-dose baseline on day 7 after dosing. Monitoring of plasma PCSK9 levels at timepoints up to the conclusion of the study on day 56 showed that some of the siRNAs, e.g. D4, had longer durability of action compared to D1. On day 56 post-dose, mice treated with D4 had lower liver PCSK9 mRNA level, higher liver siRNA level, and higher amount of siRNA associated with Argonaute-2 (Ago2) in the liver (i.e., had higher liver RISC loading) compared to mice treated with D1 (FIG. 1).

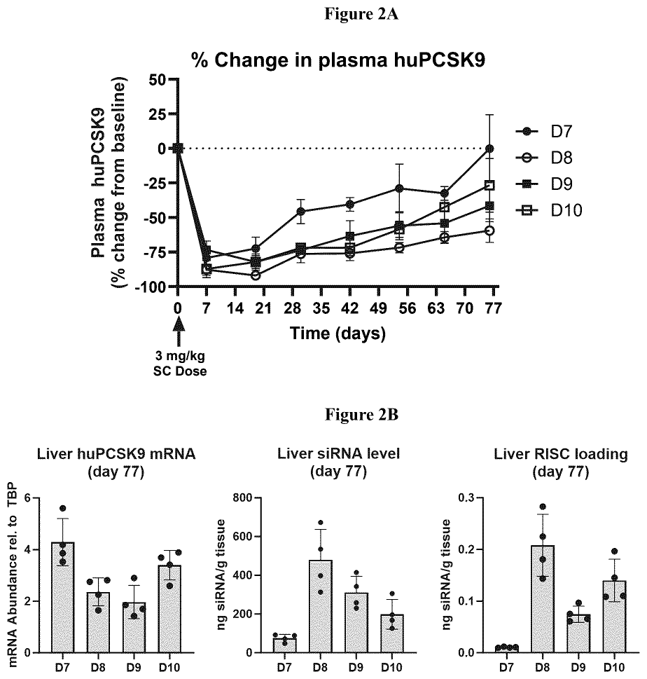

In B6-huPCSK9-UTR mice, administration (3 mg/kg subcutaneous injection) of inclisiran (D7) or D8, an siRNA with the same structure as D4, except for a different ligand including a GalNAc moiety (i.e., the same GalNAc ligand as in D7), D9 or D10 resulted in similar reductions of plasma PCSK9 levels on day 7 post-dose. Monitoring of plasma PCSK9 levels over time showed that D8, D9, and D10 administration resulted in more durable plasma PCSK9 suppression compared to inclisiran (D7) (FIG. 2A). On day 77 post-dose, D8 administration resulted in greater liver PCSK9 mRNA suppression, higher liver siRNA concentration, and higher liver RISC loading compared to mice administered inclisiran (D7) (FIG. 2B).

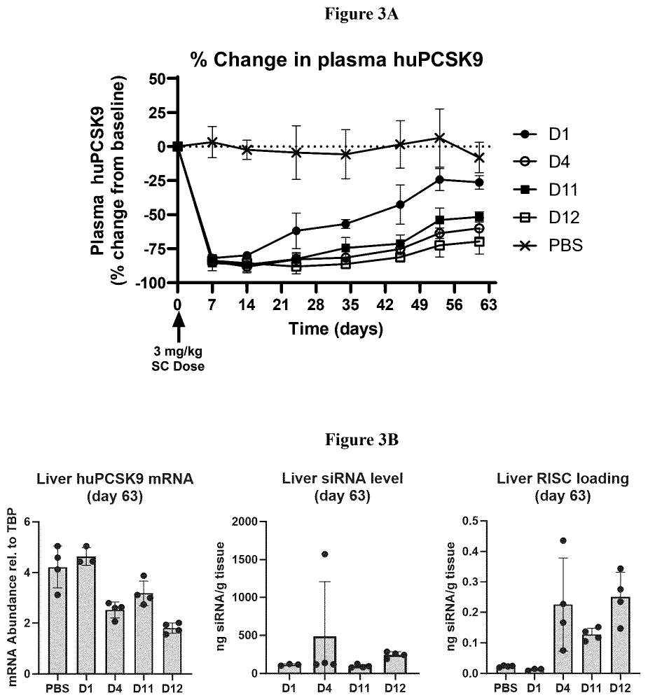

In B6-huPCSK9-UTR mice, administration (3 mg/kg subcutaneous injection) of the siRNAs D1, D4, D11, and D12 resulted in similar extents of plasma PCSK9 reduction from baseline on day 7 post-dose. As observed in a previous study (see FIG. 1A), administration of D4 resulted in more durable plasma PCSK9 suppression compared to D1 (FIG. 3A). Compared to D11 administration, administration of D12 (which has the same structure as D11 except for the addition of two phosphorothioate linkages) resulted in more durable plasma PCSK9 suppression (FIG. 3A) and lower liver PCSK9 mRNA level, higher liver siRNA concentration, and higher liver RISC loading on day 63 post-dose (FIG. 3B).

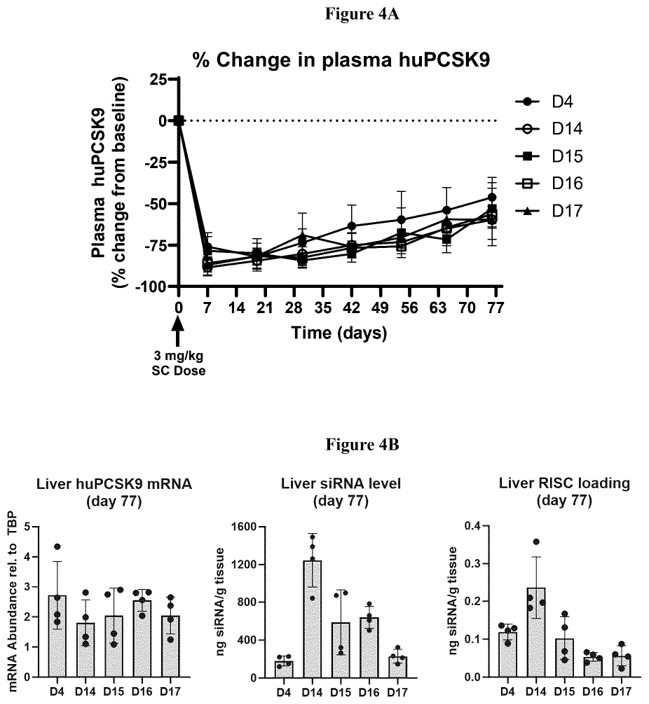

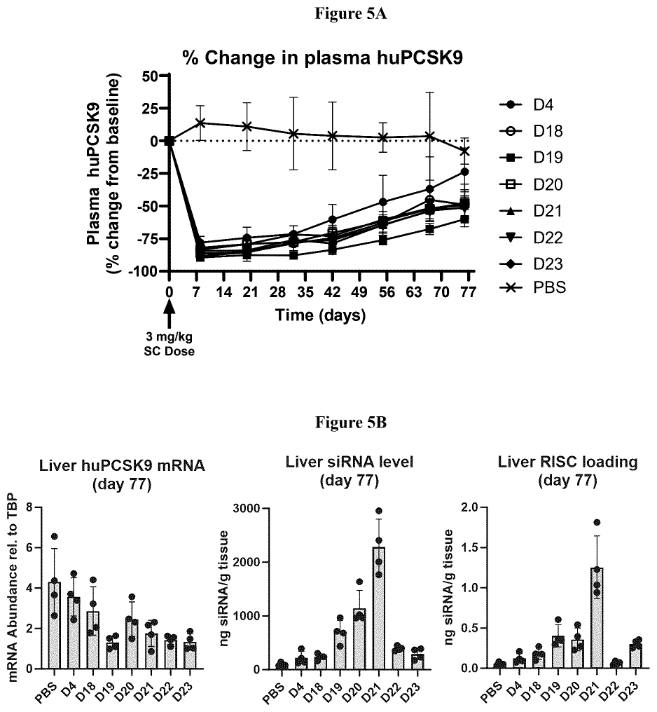

Administration of the siRNAs D4, D14, D15, D16 or D17 resulted in similar decreases in plasma PCSK9 levels on day 7 post-dose (FIG. 4A). Compared to administration of D4, administration of D14 (an siRNA with the same structure as D4 except for the 2 additional phosphorothioate linkages), D15, D16 or D17 resulted in more durable plasma PCSK9 suppression from baseline over time (FIG. 4A). Compared to D4, D14 administration resulted in greater liver PCSK9 mRNA suppression, increased liver siRNA concentration, and increased liver RISC loading on day 77 post-dose (FIG. 4B).

Administration of the siRNAs D4, D18, D19, D20, D21, D22, or D23 to B6-huPCSK9-UTR mice resulted in similar plasma PCSK9 reduction from baseline on day 7 post-dose (FIG. 5A). Monitoring of plasma PCSK9 change from baseline over time showed that, compared to D4, administration of D18, D19, D20, D21, D22 or D23 resulted in more durable plasma PCSK9 suppression (FIG. 5A). Compared to D4, D19 and D21 administration resulted in lower liver PCSK9 mRNA level, higher liver siRNA concentration, and higher liver RISC loading on day 77 post-dose (FIG. 5B).

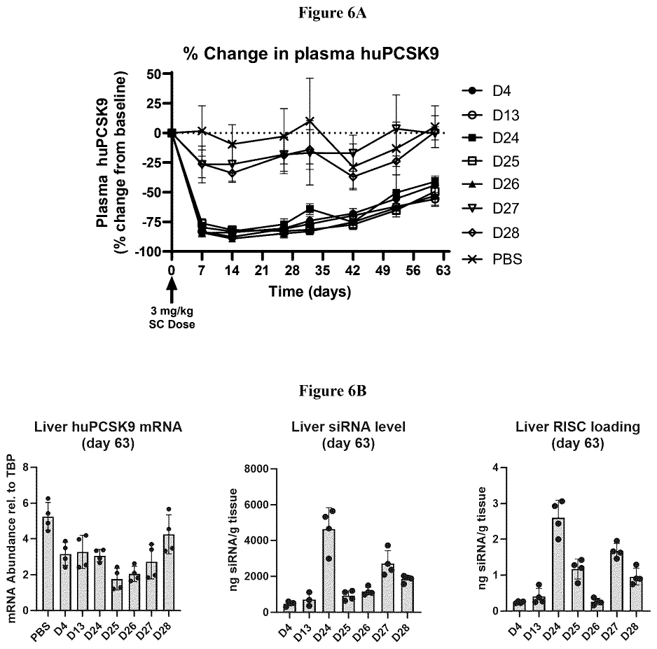

The PK/PD activities in B6-huPCSK9-UTR mice of seven siRNAs (D4, D13, D24, D25, D26, D27, and D28) were assessed. Compared to D4, administration of 2 of the siRNAs (D27 and D28) resulted in much less suppression of plasma PCSK9 levels on day 7 post-dose and over time during the course of the study (FIG. 6A). The siRNAs D13, D24, D25, and D26 had similar suppression of plasma PCSK9 levels on day 7 and similar durability of plasma PCSK9 suppression as D4. The effects of these siRNAs on liver PCSK9 mRNA levels, liver siRNA concentrations, and liver RISC loading on day 63 post-dose are shown in FIG. 6B.

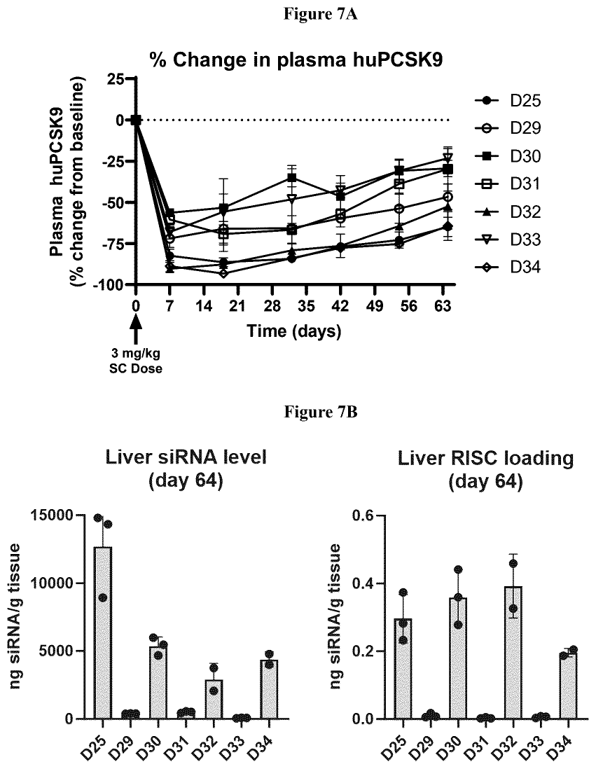

The PK/PD activities in B6-huPCSK9-UTR mice of 7 siRNAs with different nucleotide sequences than inclisiran (D25, D29, D30, D31, D32, D33, and D34) were evaluated. Several of the siRNAs (D29, D30, D31, and D33) caused less suppression of plasma PCSK9 at day 7 post-dose compared to the D25, D32 and D34, which caused similar plasma PCSK9 suppression on day 7 post-dose. Among the siRNAs in this study, D25 and D34 administration resulted in the greatest suppression of plasma PCSK9 on day 64 post-dose (FIG. 7A). The effects of these siRNAs on liver PCSK9 mRNA levels, liver siRNA concentrations, and liver RISC loading on day 64 post-dose are shown in FIG. 7B.

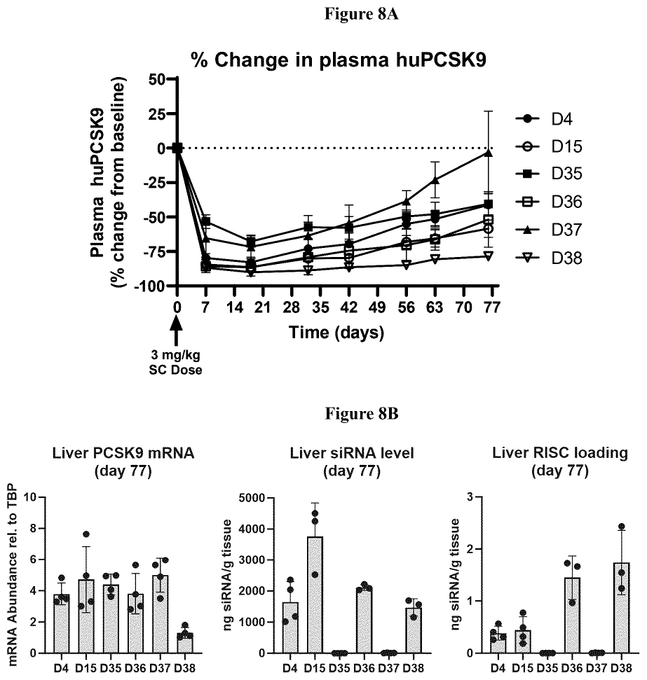

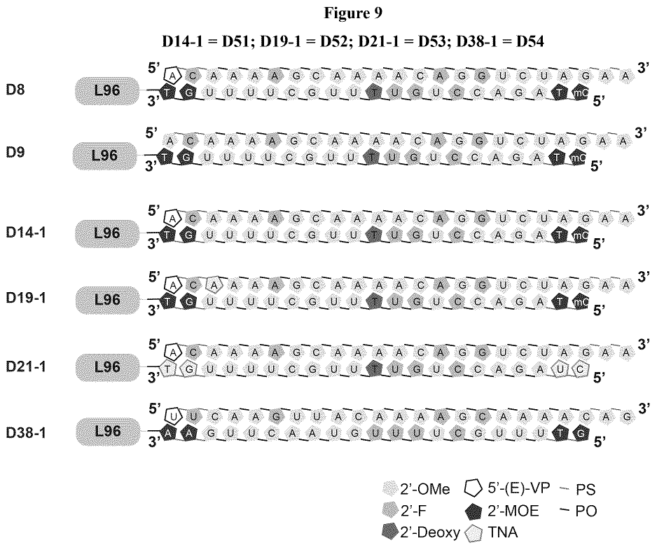

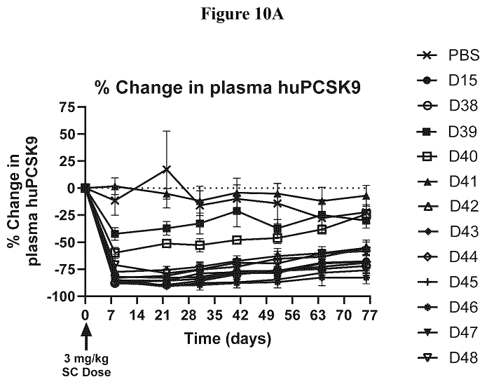

The siRNAs D4, D15, D35, D36, D37 and D38, which have various nucleotide sequences, were administered to B6-huPCSK9-UTR mice. Among these siRNAs, D15, D36 and D38 caused the greatest suppression of plasma PCSK9 levels relative to baseline on day 76 post-dose (FIG. 8A). Compared to D4, administration of D38 resulted in greater liver PCSK9 mRNA reduction, similar liver siRNA concentrations, and greater liver RISC loading on day 77 post-dose (FIG. 8B).

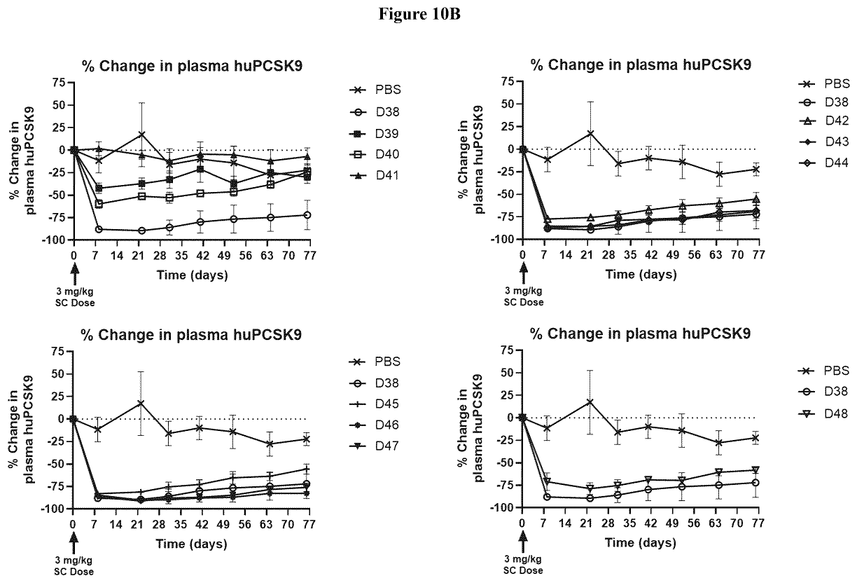

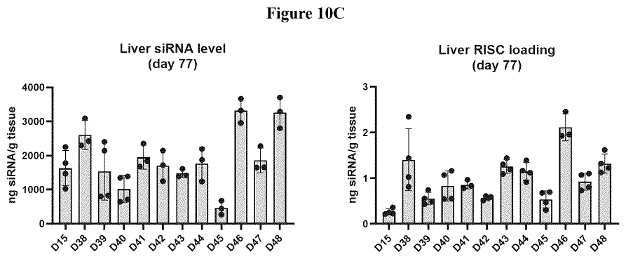

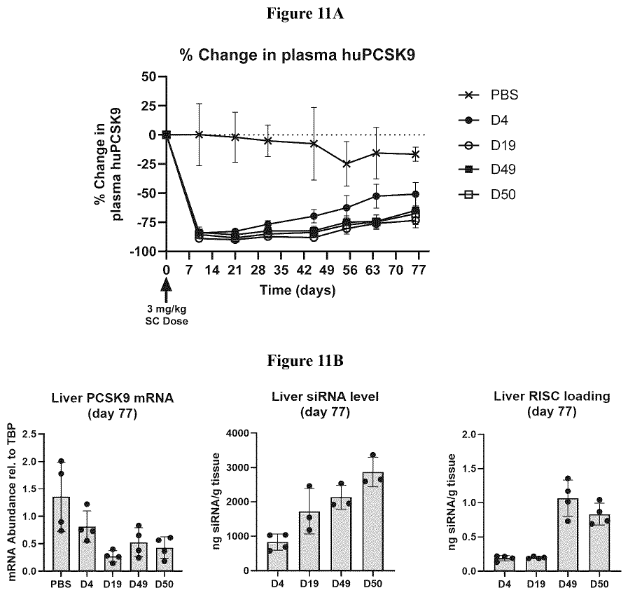

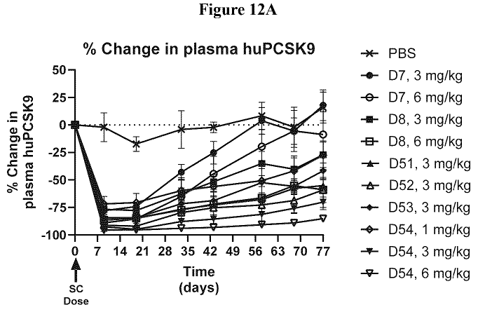

The siRNAs D15, D38, D39, D40, D41, D42, D43, D44, D45, D46, D4, and D48 were administered via a single 3 mg/kg subcutaneous injection to B6-huPCSK9-UTR mice. The effects of the siRNAs on plasma PCSK9 levels up to day 77 post-dose are shown in FIG. 10A; for clarity, subsets of this data are shown in separate graphs in FIG. 10B. D39, D40, and D41, which have a GNA nucleotide in position 5, 6, and 7, respectively, of the antisense strand, had reduced PCSK9 suppression compared to D38 at all post-dose timepoints (FIG. 10B). D42, D43, and D44, which have a TNA nucleotide in position 5, 6, and 7, respectively, of the antisense strand, caused similar PCSK9 suppression compared to D38 at all post-dose timepoints (FIG. 10B). D45, D46, and D47, which have a DNA nucleotide in position 5, 6, and 7, respectively, of the antisense strand, caused similar PCSK9 suppression at all post-dose timepoints (FIG. 10B). D48, which has a 2′-OMe-U nucleotide at position 3 of the antisense strand and a 2′-OMe-A at position 19 of the sense strand, caused similar PCSK9 suppression at all post-dose timepoints (FIG. 10B). All siRNAs included in this study showed detectable liver siRNA and Liver RISC loading on day 77 post-dose (FIG. 10C).

The siRNAs D4, D19, D49, and D50 were administered via a single 3 mg/kg subcutaneous injection to B6-huPCSK9-UTR mice. As observed in another study (see FIG. 5A), D19 administration suppressed plasma PCSK9 more durably compared to D4 (FIG. 11A). D49 and D50 administration suppressed plasma PCSK9 more durably compared to D4, and similarly to D19 at all post-dose timepoints (FIG. 11A). Consistent with the greater plasma PCSK9 suppression observed on day 77 post-dose for D19, D49, and D50 compared to D4 (FIG. 11A), D19, D49, and D50 suppressed liver PCSK9 mRNA on day 77 post-dose to a greater extent than D4 (FIG. 11B). Liver siRNA level on day 77 post-dose was higher for D19, D49, and D50 compared to D4 (FIG. 11B). Liver RISC loading on day 77 post-dose was higher for D49 and D50 compared to D4 and D19 (FIG. 11B).

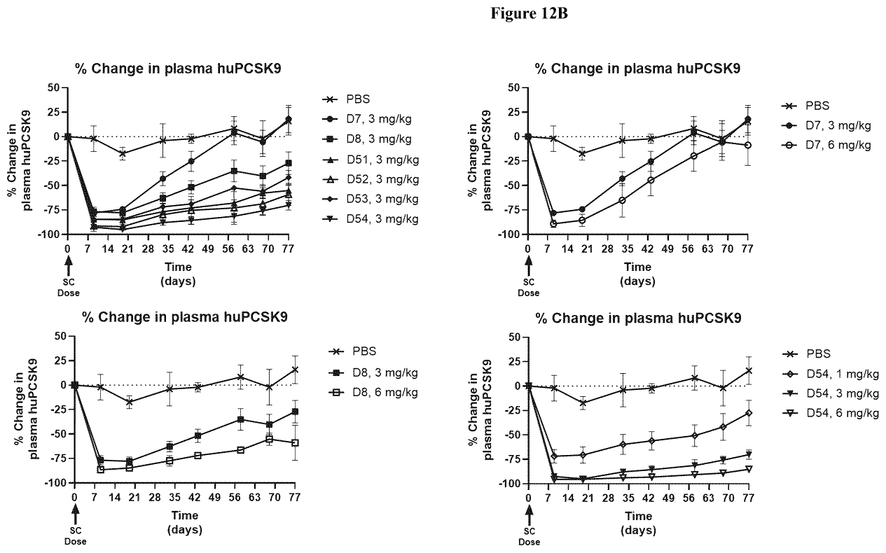

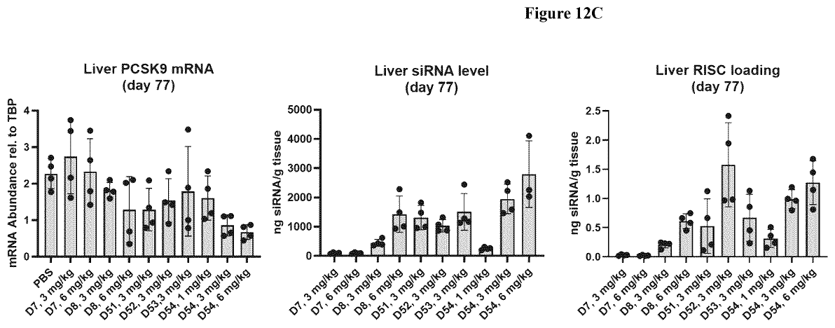

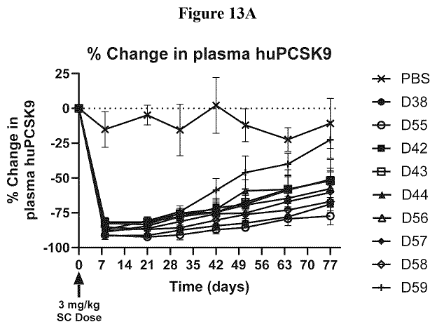

The siRNAs D7, D8, D51, D52, D53, and D54 were administered to B6-huPCSK9-UTR mice as a single subcutaneous injection at the following dose levels: 3 mg/kg for all siRNAs, also at 6 mg/kg for D7, D8, and D54, and also at 1 mg/kg for D54. The effects of siRNA administration on plasma PCSK9 levels at up to 77 days post-dose are shown in FIG. 12A; for clarity, subsets of this data are replotted in FIG. 12B. At a dose level of 3 mg/kg, plasma PCSK9 suppression was more durable for D8 compared to D7, and greater plasma PCSK9 suppression was seen at all post-dose timepoints for D51, D52, D53, and D54 compared to D8 (FIG. 12B). D7, D8, and D54, for which more than one dose level was evaluated in this study, showed dose-dependent plasma PCSK9 suppression (FIG. 12B). The magnitude of plasma PCSK9 suppression at the first post-dose timepoint (day 9) is shown for each siRNA and dose level in Table 11; at the 3 mg/kg dose level, D52 and D54 had the greatest plasma PCSK9 suppression on day 9 post-dose (91.2% and 92.6%, respectively). At day 77 post-dose, for the 3 mg/kg dose level, liver PCSK9 mRNA suppression by D8, D51, D52, D53, and D54 was greater compared to D7, for which the liver PCSK9 mRNA level was equivalent to that of vehicle-treated mice (vehicle: PBS, phosphate-buffered saline) (FIG. 12C). The liver siRNA level on day 77 post-dose for the 3 mg/kg dose level was lowest for D7, intermediate for D8, and highest for D51, D52, D53, and D54 (FIG. 12C). The liver RISC loading level on day 77 post-dose for the 3 mg/kg dose level was lowest for D7, higher for D8, still higher for D51 and D53, and highest for D52 and D54.

TABLE 11

| | | % Change from baseline in |
|---|---|---|
| | | plasma PCSK9 at first post- |
| | Dose | dose timepoint (day 9) |
| siRNA | (mg/kg) | (mean ± standard deviation) |
| D 7 | 3 | −78.0 ± 2.4 (n = 4) |
| D 7 | 6 | −89.0 ± 3.7 (n = 4) |
| D 8 | 3 | −76.7 ± 4.7 (n = 4) |
| D 8 | 6 | −86.4 ± 3.7 (n = 4) |
| D 51 | 3 | −84.3 ± 2.1 (n = 4) |
| D 52 | 3 | −91.2 ± 5.6 (n = 4) |
| D 53 | 3 | −84.5 ± 3.2 (n = 4) |
| D 54 | 1 | −71.9 ± 7.0 (n = 4) |
| D 54 | 3 | −92.6 ± 3.2 (n = 4) |
| D 54 | 6 | −95.5 ± 1.2 (n = 4) |

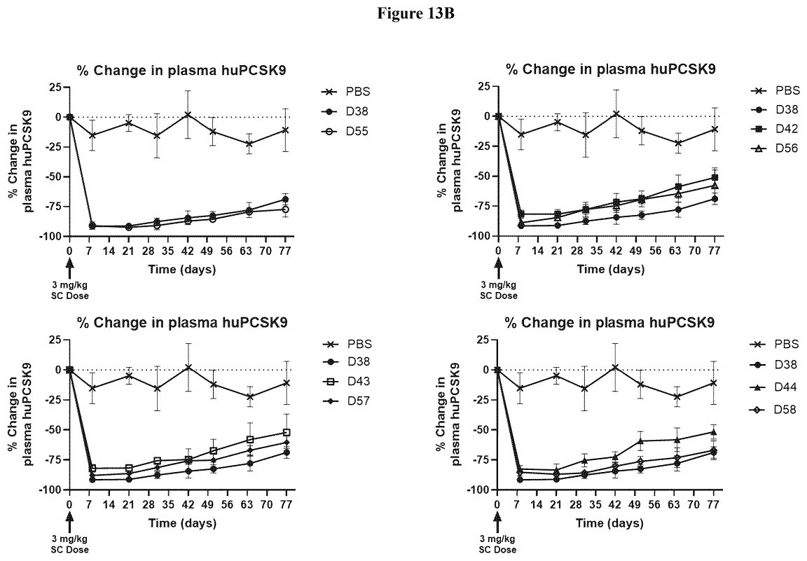

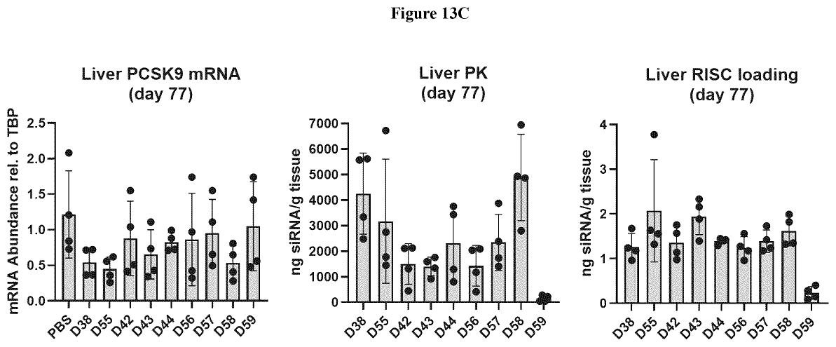

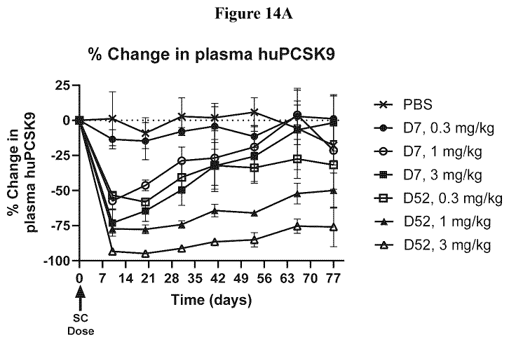

The siRNAs D38, D42, D43, D44, D55, D56, D57, D58, and D59 were administered to B6-huPCSK9-UTR mice as a single 3 mg/kg subcutaneous injection. The effects of siRNA administration on plasma PCSK9 monitored at timepoints up to day 77 post-dose are shown in FIG. 13A; for clarity, subsets of these data are replotted in FIG. 13B. Compared to D38, D55, which is an analog of D38 with a TNA nucleotide at position 3 of the antisense strand, had a similar effect on plasma PCSK9 levels over time (FIG. 13B). Compared to D38, analogs D42, D43, and D44, which have a TNA nucleotide in position 5, 6, or 7, respectively, of the antisense strand, had slightly less potent and/or durable suppression of plasma PCSK9 over the course of the study. Compared to D38, the analogs D56, D57, and D58 with TNA nucleotides in position 3 of the antisense strand and in position 5, 6, or 7, respectively, of the antisense strand, had similarly potent plasma PCSK9 suppression at the first post-dose timepoint (day 8 post-dose) and had durability of plasma PCSK9 suppression over the course of the study that was slightly lower (for D56) or similar (for D57 and D58) (FIG. 13B). The effects of all siRNAs in this study on liver PCSK9 mRNA level, liver siRNA concentration, and liver RISC loading on day 77 post-dose are shown in FIG. 13C.

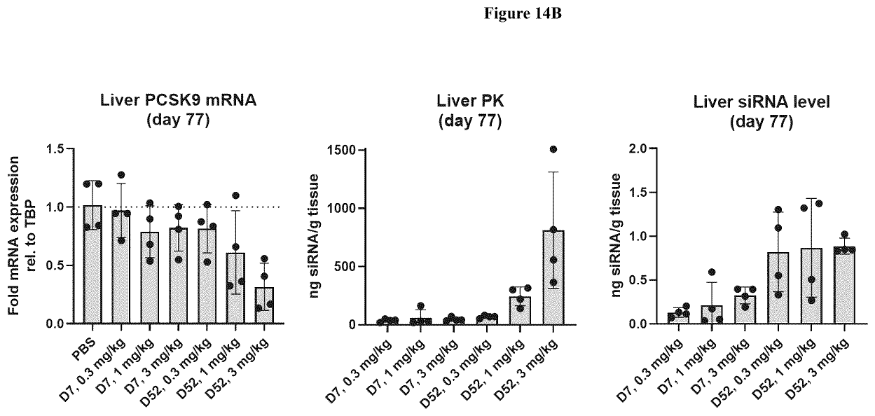

The siRNAs D7 and D52 were administered as a single subcutaneous injection to B6-huPCSK9-UTR mice at a dose level of 0.3, 1, or 3 mg/kg. Both siRNAs dose-dependently suppressed plasma PCSK9 (FIG. 14A). At each dose level, plasma PCSK9 suppression at the first post-dose timepoint (day 10) was greater for D52 compared to D7 (FIG. 14A, Table 12). In addition, plasma PCSK9 suppression over time was more durable at each dose level for D52 compared to D7. These results indicate that D52 has higher in vivo potency and durability of action compared to D7. Consistent with the plasma PCSK9 results, on day 77 post-dose D52 at the 3 mg/kg dose level suppressed liver PCSK9 mRNA to a greater extent than D7 at the 3 mg/kg dose level (FIG. 14B). On day 77 post-dose, D52 at the 3 mg/kg dose level had higher liver siRNA concentration and higher liver RISC loading compared to D7 at the 3 mg/kg dose level (FIG. 14B).

TABLE 12

| | | % Change from baseline in |
|---|---|---|
| | | plasma PCSK9 at first post- |
| | Dose | dose timepoint (day 10) |
| siRNA | (mg/kg) | (mean ±/− standard deviation) |
| D 7 | 0.3 | −13.5 ± 6.8 (n = 4) |
| D 7 | 1 | −57.5 ± 5.6 (n = 4) |
| D 7 | 3 | −73.1 ± 9.4 (n = 4) |
| D 52 | 0.3 | −53.4 ± 2.4 (n = 4) |
| D 52 | 1 | −77.3 ± 3.1 (n = 4) |
| D 52 | 3 | −93.3 ± 2.2 (n = 4) |

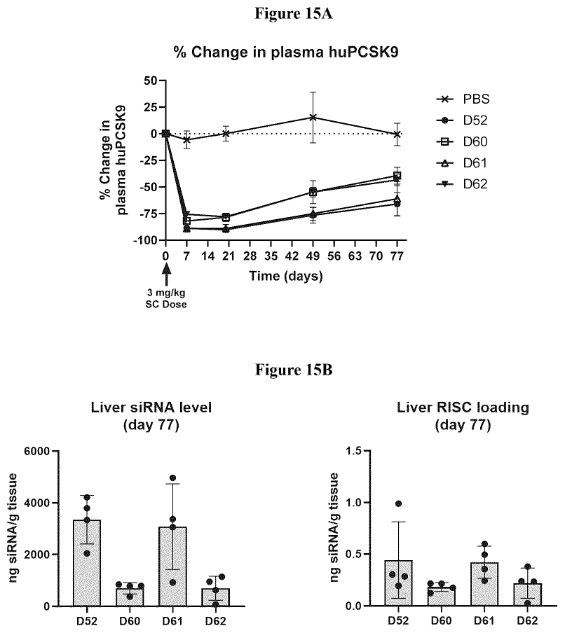

The siRNAs D52, D60, D61, and D62 were administered via a single 3 mg/kg subcutaneous injection to B6-huPCSK9-UTR mice. At all post-dose timepoints, D61 suppressed plasma PCSK9 to the same extent as D52; in contrast D60 and D62 suppressed plasma PCSK9 between day 21 and day 77 to a lesser extent compared to D52 (FIG. 15A). On day 77 post-dose, liver siRNA concentrations and liver RISC loading levels were similar for D52 and D61 and lower for D60 and D62 (FIG. 15B).

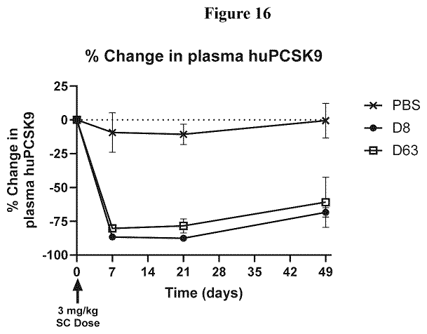

The siRNAs D8 and D63 were administered via a single 3 mg/kg subcutaneous injection to B6-huPCSK9-UTR mice. At all post-dose timepoints, D63 suppressed plasma PCSK9 to a similar extent as D8 (FIG. 16).

### Example 5. In Vitro Dose-Dependent Profiling in Hep3B Cells

Hep3B cells (ATCC. Cat. HB-8064) were cultured in EMEM with L-Glut (ATCC 30-2003)+10% FBS medium (heat inactivated). Cells were seeded in a 96-well plate and reverse transfected with siRNA D7, D8, or D52 using RNAiMax (Thermofisher cat. 13778150) in an 8-point dose response starting with 5 nM siRNA using a 1:4 dilution factor. Remaining PCSK9 mRNA levels were measured 24 hr post transfection by qPCR using PCSK9 and PGK1 taqman assays. The degree of knock-down was calculated using the delta delta CT method. Degree of knock-down vs. siRNA concentration data was used to determine the IC50 and maximum inhibition (%).

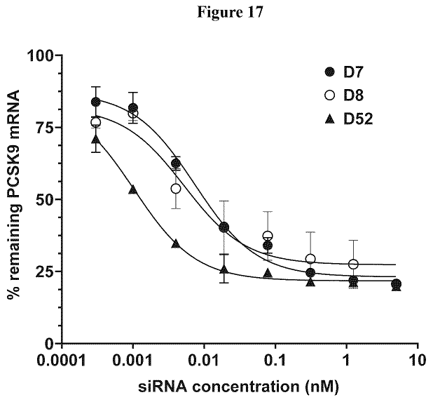

Dose response curves for D7, D8, and D52 are shown in FIG. 17 and IC50 and maximum inhibition values are shown in Table 13. All 3 siRNAs had similar maximum PCSK9 mRNA inhibition (Table 13). Compared to D7, the IC50 values for D8 and D52 were 1.4-fold lower and 7.2-fold lower, respectively. These results indicate that D52 has significantly higher in vitro potency compared to D7 and D8.

TABLE 13

| siRNA | IC50 (nM) | Max inhibition (%) |
|---|---|---|
| D 7 | 0.00818 | 79.22 |
| D 8 | 0.00568 | 79.35 |
| D 52 | 0.00113 | 80.15 |

### Example 6. Specificity Profiling in Hep3B Cells

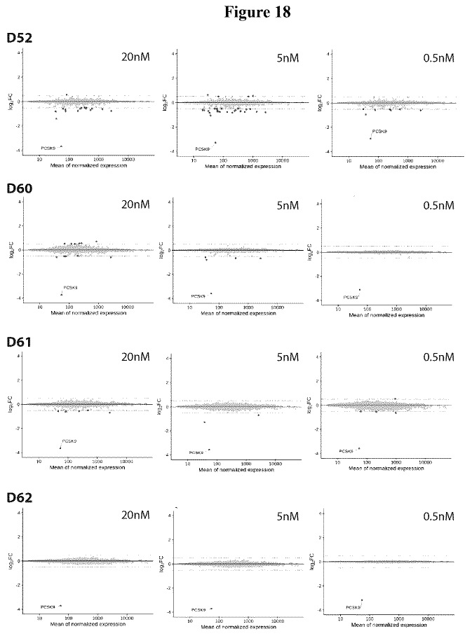

Hep3B cells (ATCC. Cat. HB-8064) were cultured in EMEM with L-Glut (ATCC 30-2003)+10% FBS medium (ATCC. Cat. 30-2020). Cells were seeded in a 96-well plate and reverse transfected with an siRNA concentration of 20 nM, 5 nM or 0.5 nM for D52, D60, D61 or D62 using RNAiMax (Thermofisher cat. 13778150). Cells are harvested, lysed and total RNA is extracted using MagMAX™-96 Total RNA Isolation Kit (Thermofisher Cat. AM1830) 24 hrs post transfection. Transcriptome-wide changes of cells treated with siRNA were characterized using a high-throughput 3′mRNA sequencing technology relative to cells treated with vehicle control (PBS+RNAiMax). Differential gene expression is displayed using MA-plots for each siRNA (i.e. D52, D60, D61, D62) at a given concentration, highlighting the on-target knockdown for PCSK9 (FIG. 18).

### Example 7. In Vivo Profiling in Cynomolgus Monkeys

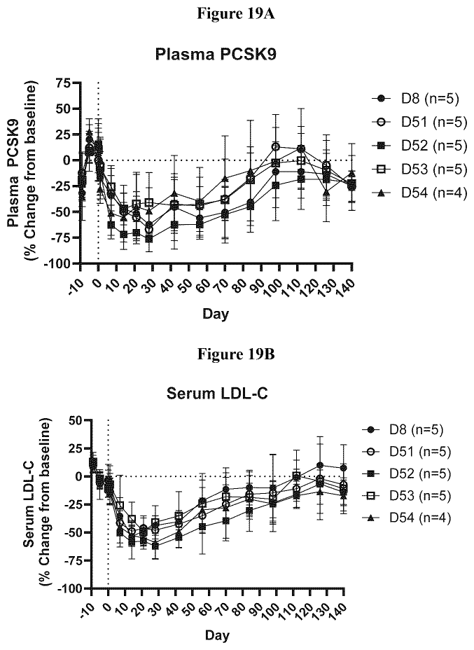

The siRNAs D8, D51, D52, D53, and D54 were administered to obese cynomolgus monkeys (n=5 for each siRNA) via a single 3 mg/kg subcutaneous injection. Group mean plasma siRNA concentrations measured by liquid chromatography mass spectrometry (LC-MS) at 2, 4, and 24 hr post-dose are listed in Table 14; note that one monkey in the D54 group had undetectable plasma siRNA concentration at all three timepoints and was therefore excluded from the group mean plasma siRNA calculation and from group mean plasma PCSK9 and serum LDL-C calculations. PCSK9 protein concentrations in plasma samples were determined using a human PCSK9 ELISA kit from R&D Systems (catalog no. SPC900) with purified, recombinant cynomolgus monkey PCSK9 (prepared at Novartis; stock solution concentration 0.9 mg/mL) used as the standard. Plasma PCSK9 concentrations and serum LDL-C concentrations were monitored at several pre-dose timepoints and at post-dose timepoints up to 140 days post-dose (FIG. 19). All of the siRNAs in this study suppressed plasma PCSK9 and serum LDL-C levels relative to baseline values, with group mean plasma PCSK9 and serum LDL-C nadirs occurring on days 14-28 post-dose (FIG. 19, Table 15) followed by a gradual return to baseline levels (FIG. 19). Compared to D8, D52 had greater maximal plasma PCSK9 lowering (76.20 vs. 62.2%) and greater maximal LDL-C lowering (62.2% vs. 53.5%) (Table 15). D52 also reduced serum LDL-C to a greater extent than D8 at multiple timepoints after its LDL-C lowering nadir on day 28; for example, on day 70 post-dose, serum LDL-C was lowered by 39.6 h and 11.5 for D52 and D8, respectively (FIG. 19B).

TABLE 14

Plasma siRNA concentration (ng/mL, group
mean ± standard deviation)

| | 2 hr | 4 hr | 24 hr |
|---|---|---|---|
| siRNA | post-dose | post-dose | post-dose |
| D 8 (n = 5) | 2036 ± 561 | 1681 ± 498 | 12 ± 12 |
| D 51 (n = 5) | 1189 ± 383 | 1042 ± 335 | 6 ± 1 |
| D 52 (n = 5) | 2054 ± 565 | 1548 ± 481 | 5 ± 1 |
| D 53 (n = 5) | 1506 ± 586 | 1245 ± 628 | 6 ± 2 |
| D 54 (n = 4) | 1971 ± 1111 | 1283 ± 308 | 5 ± 2 |

TABLE 15

| | Maximal plasma PCSK9 | Maximal serum LDL-C |
|---|---|---|
| siRNA | % change from baseline | % change from baseline |
| D 8 | −62.2 ± 17.3 (on day 28), n = 5 | −53.5 ± 9.0 (on day 14), n = 5 |
| D 51 | −67.0 ± 5.7 (on day 28), n = 5 | −48.8 ± 9.6 (on day 14), n = 5 |
| D 52 | −76.2 ± 12.3 (on day 28), n = 5 | −62.2 ± 11.5 (on day 28), n = 4 |
| D 53 | −47.2 ± 15.5 (on day 14), n = 5 | −49.3 ± 12.3 (on day 21), n = 5 |
| D 54 | −55.5 ± 23.7 (on day 14), n = 4 | −58.8 ± 14.5 (on day 28), n = 4 |

1. A double stranded RNAi (dsRNAi) agent comprising:
(i) a sense strand comprising a nucleotide sequence selected from SEQ ID Nos. 3 to 381 and SEQ ID Nos. 761 to 776; and
(ii) an antisense strand forming a duplex with the sense strand and comprising a nucleotide sequence selected from SEQ ID Nos. 382 to 760 and SEQ ID Nos. 777 to 792.

2. (canceled)

3. The dsRNAi agent of claim 1, wherein one or more nucleotides in the sense strand and the antisense strand are modified nucleotides.

4.-6. (canceled)

7. The dsRNAi agent of claim 3, wherein each of the modified nucleotides independently comprises one or more modifications selected from 2′-deoxy modification, 2′-O-methyl (2′-OMe) modification, 2′-fluoro (2′-F) modification, 2′-O-methoxyethyl (2′-MOE) modification, the modification containing a non-natural nucleobase, TNA, GNA, 3′-phosphorothioate (PS) modification, and 5′-vinyl-phosphonate (5′-VP) modification.

8. The dsRNAi agent of claim 7, wherein the sense strand comprises one or two 2′-MOE modifications positioned at the 1st and/or 2nd nucleotides from the 5′ end; and/or wherein the sense strand comprises one or two 2′-MOE modifications positioned at the 1st and/or 2nd nucleotides from the 3′ end.

9.-11. (canceled)

12. The dsRNAi agent of claim 3, wherein the antisense strand comprises a 5′-VP modification at the 1st nucleotide from the 5′ end.

13.-39. (canceled)

40. A double stranded RNAi (dsRNAi) agent comprising:
(a) a sense strand comprising SEQ ID NO: 800, and
an antisense strand comprising SEQ ID NO: 853;
(b) a sense strand comprising SEQ ID NO: 801, and
an antisense strand comprising SEQ ID NO: 854;
(c) a sense strand comprising SEQ ID NO: 806, and
an antisense strand comprising SEQ ID NO: 859;
(d) a sense strand comprising SEQ ID NO: 811, and
an antisense strand comprising SEQ ID NO: 864;
(e) a sense strand comprising SEQ ID NO: 813, and
an antisense strand comprising SEQ ID NO: 866;
(f) a sense strand comprising SEQ ID NO: 830, and
an antisense strand comprising SEQ ID NO: 883; or
(g) a sense strand comprising SEQ ID NO: 1016, and
an antisense strand comprising SEQ ID NO: 1022.

41. The dsRNAi agent of claim 40, further comprising a ligand.

42. The dsRNAi agent of claim 1, wherein the ligand comprises a N-acetylgalactosamine (GalNAc) moiety.

43. The dsRNAi agent of claim 42, wherein the ligand has a structure of: 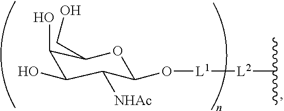
wherein:
each L¹ is independently a linker which may be same or different in each occurrence;
L² is a linker;
n is an integer from 1 to 3; and [symbol]
is an attachment point to the sense strand or the antisense strand, or to a conjugate linker conjugated to the sense strand or the antisense strand.

44.-46. (canceled)

47. The dsRNAi agent of claim 43, wherein the ligand comprises the following structure: 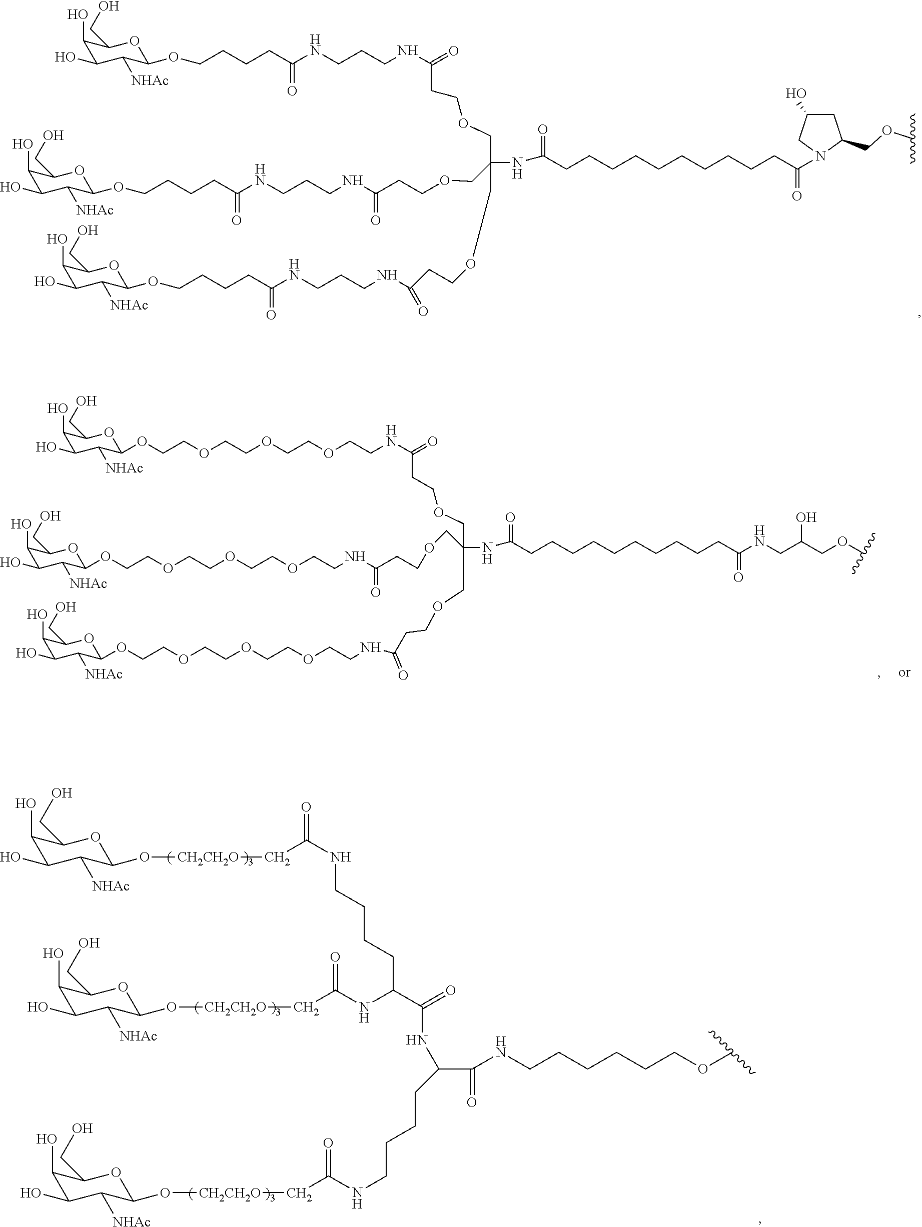
wherein [symbol]
is an attachment point to the sense strand or the antisense strand or to a conjugate linker conjugated to the sense strand or the antisense strand.

48. The dsRNAi agent of claim 47, wherein the ligand is conjugated to 3′ end of the sense strand to form the following structure: 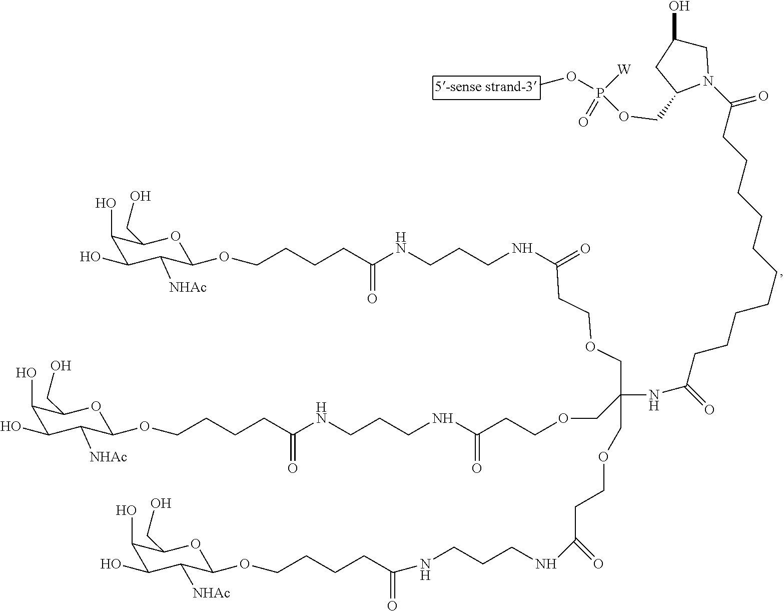
wherein W is —OH or —SH.

49. (canceled)

50. The dsRNAi agent of claim 48, wherein W is —OH.

51. A double stranded RNAi (dsRNAi) agent comprising:
(a) a sense strand consisting of SEQ ID NO: 906, and
an antisense strand consisting of SEQ ID NO: 963;
(b) a sense strand consisting of SEQ ID NO: 907, and
an antisense strand consisting of SEQ ID NO: 964;
(c) a sense strand consisting of SEQ ID NO: 947, and
an antisense strand consisting of SEQ ID NO: 1004;
(d) a sense strand consisting of SEQ ID NO: 948, and
an antisense strand consisting of SEQ ID NO: 1005;
(e) a sense strand consisting of SEQ ID NO: 949, and
an antisense strand consisting of SEQ ID NO: 1006;
(f) a sense strand consisting of SEQ ID NO: 950, and
an antisense strand consisting of SEQ ID NO: 1007; or
(g) a sense strand consisting of SEQ ID NO: 1028, and
an antisense strand consisting of SEQ ID NO: 1034
wherein the ligand (L96) is conjugated to the 3′ end of the sense strand to form the following schematic: 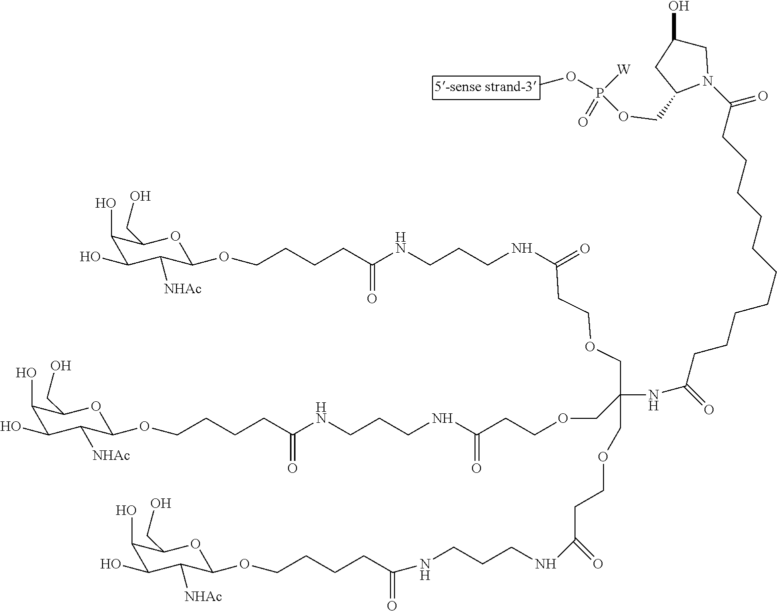
or a pharmaceutically acceptable salt thereof, wherein W is —OH.

52. The dsRNAi agent of claim 1, wherein the dsRNAi agent is in a pharmaceutically acceptable salt form.

53. The dsRNAi agent of claim 52, wherein the pharmaceutically acceptable salt is a sodium salt.

54.-55. (canceled)

56. A method of inhibiting PCSK9 expression in a cell, the method comprising:
(a) contacting the cell with the dsRNAi agent of claim 1 or a pharmaceutically acceptable salt thereof, and
(b) maintaining the cell produced in step (a) for a time sufficient to obtain degradation of the mRNA transcript of a PCSK9 gene, thereby inhibiting expression of the PCSK9 gene in the cell.

57. A method of lowering a level of low-density lipoprotein cholesterol (LDL-C) in a subject in need thereof, comprising administering to the subject the dsRNAi agent of claim 1 or a pharmaceutically acceptable salt thereof.

58. A method of treating lipidemia mediated by PCSK9 expression in a subject in need thereof, comprising administering to the subject the dsRNAi agent of claim 1 or a pharmaceutically acceptable salt thereof.

59. A method of treating or preventing atherosclerotic cardiovascular disease (ASCVD) in a subject in need thereof, comprising administering to the subject the dsRNAi agent of claim 1 or a pharmaceutically acceptable salt thereof.

60. A method of reducing or preventing cardiovascular event in a subject in need thereof, comprising administering to the subject the dsRNAi agent of claim 1 or a pharmaceutically acceptable salt thereof.

61. The method of claim 60, wherein the cardiovascular event is cardiovascular death, non-fatal myocardial infarction (MI), non-fatal ischemic stroke, urgent coronary revascularization, coronary heart disease (CHD) death, or any combination thereof.

62. A method of reducing or preventing a major limb adverse event (MALE) in a subject in need thereof, comprising administering to the subject the dsRNAi agent of claim 1 or a pharmaceutically acceptable salt thereof.

63. The method of claim 62, wherein the MALE is acute lower limb ischemia, lower limb amputation due to ischemia, urgent lower limb revascularization for ischemia, or any combination thereof.

64.-75. (canceled)

76. A kit comprising the dsRNAi agent of claim 1 or a pharmaceutically acceptable salt thereof.

77.-79. (canceled)
